(19) United States
(12) Patent Application Publication
(10) Pub. No.: US 2023/0272041 A1
(43) Pub. Date: Aug. 31, 2023

(54) Formulation, Dosage Regimen, and Manufacturing Process for Heterodimeric FC-Fused Proteins

(71) Applicant: Dragonfly Therapeutics, Inc., Waltham, MA (US)

(72) Inventors: Mitchell Bigelow, Cambridge, MA (US); Alexandra Braun, Cambridge, MA (US); Ann F. Cheung, Lincoln, MA (US); Jean-Marie Cuillerot, Somerville, MA (US); Mark Derose, Wilmington, MA (US); Asya Grinberg, Lexington, MA (US); Eva Gutierrez, Waltham, MA (US); Patrick Kirby, Boxborough, MA (US); Christopher Ryan Morgan, Southborough, MA (US); Michael C. Naill, Stow, MA (US); Steven O'Neil, Wayland, MA (US); Michael Shifrin, Medford, MA (US); Nicolai Wagtmann, Concord, MA (US)

(21) Appl. No.: 17/920,174

(22) PCT Filed: Apr. 22, 2021
(86) PCT No.: PCT/US2021/028701

§ 371 Date: Oct. 20, 2022

Related U.S. Application Data
(60) Provisional application No. 63/013,834, filed on Apr. 22, 2020.
(60) Provisional application No. 63/033,161, filed on Jun. 1, 2020.

(51) Int. Cl. C07K 14/735; A61K 31/7016; A61K 47/14; A61K 9/00; A61P 35/04; C07K 16/28; A61K 38/20; A61K 9/19

(52) U.S. Cl. CPC C07K 14/70535; A61K 31/7016; A61K 47/14; A61K 9/0019; A61P 35/04; C07K 16/2896; A61K 38/208; A61K 9/19

(57) ABSTRACT

The present invention relates to pharmaceutical formulations for heterodimeric Fc-fused proteins which are advantageous for achieving higher titers of the proteins during production, higher stability during storage, and improved efficacy when used as a therapeutic. Also provided are dosage regimens for such heterodimeric Fc-fused proteins and pharmaceutical formulations for use in treating cancer, such as locally advanced or metastatic solid tumor.

### CROSS-REFERENCE TO RELATED APPLICATIONS

This application claims priority to and benefit of U.S. Provisional Application No. 63/013,834, filed on Apr. 22, 2020, and U.S. Provisional Application No. 63/033,161, filed on Jun. 1, 2020, each of which is hereby incorporated by reference herein in its entirety.

### SEQUENCE LISTING

The content of the electronically submitted sequence listing in ASCII text file (Name: 3338_259PC03_Seglisting_ST25.txt; Size: 946,336 bytes; and Date of Creation: Apr. 21, 2021) filed with the application is incorporated herein by reference in its entirety.

### FIELD OF THE INVENTION

The present disclosure relates to pharmaceutical formulations and dosage regimens for heterodimeric Fc-fused proteins, and methods of using such proteins to treat cancers.

### BACKGROUND

Physiologically active proteins mostly have the disadvantage of having a short in vivo half-life. In order to solve this disadvantage, there has been an attempt to conjugate them to PEG (polyethylene glycol) or the like, or to fuse them to an antibody Fc (crystallizable fragment) region. Proteins composed of two or more different subunits, in which the two or more different subunits form a protein complex to exhibit physiological activity, can be fused to wild-type Fc domains to prepare Fc-fused protein forms, forming a homodimer due to the homodimeric nature of Fc. Proteins composed of two or more different subunits, in which the two or more different subunits form a protein complex to exhibit physiological activity, can also be fused to heterodimeric Fc regions derived not only from IgG1, but also from other isotype antibodies such as IgG2, IgG3 and IgG4, to form a heterodimeric Fc-fused protein. Thus, one or more subunit(s) of the protein, which is composed of two or more different subunits and in which two or more subunits exhibit physiological activity by forming a protein complex, can be fused to the terminus of heterodimeric Fc variant regions to form improved Fc-fused protein forms.

Fc heterodimerization is a technology that induces mutations in two different CH3 domains of Fc by genetic engineering, such that the two Fc fragments form a heterodimer with minimal sequence variations while they have tertiary structures very similar to those of naturally occurring antibodies (see, e.g., U.S. Pat. No. 7,695,936).

The inventions described in the present disclosure provide designs for improving the Fc-fused protein forms, in which the two subunits of a heterodimeric protein are connected to two Fc domains having different heterodimerization domains, by introducing linkers of varying lengths, or mutations in the CH2 and the CH3 domains of the Fc. Further the inventions described in the present disclosure provides pharmaceutical formulations for such proteins, method of making such proteins and formulations, and methods for treating cancer using such proteins.

### SUMMARY

The invention generally relates to pharmaceutical formulations comprising certain heterodimeric Fc-fused proteins comprising IL12 subunit(s), processes for preparing such proteins and pharmaceutical formulations. Also provided are dosage regimens for using such heterodimeric Fc-fused proteins and pharmaceutical formulations to treat cancer, such as locally advanced or metastatic solid tumors.

Accordingly, in one aspect, provided herein is a pharmaceutical formulation comprising a heterodimeric Fc-fused protein comprising a first polypeptide comprising a first antibody Fc domain polypeptide and a first subunit of a multisubunit cytokine and a second polypeptide comprising a second antibody Fc domain polypeptide and a second, different subunit of the multisubunit cytokine, citrate, a sugar, a sugar alcohol, and a non-ionic surfactant, at pH 6.0 to 7.0, wherein the first and second antibody Fc domain polypeptides each comprise different mutations promoting heterodimerization, and wherein the first subunit and second, different subunit of the multisubunit cytokine are bound to each other. In some embodiments, the first and/or second antibody Fc domain polypeptides comprise one or more mutation(s) that reduce(s) an effector function of an Fc.

In some embodiments, the concentration of citrate in the pharmaceutical formulation is about 10 mM to about 30 mM. In certain embodiments, the concentration of citrate in the pharmaceutical formulation is about 20 mM. In some embodiments, the concentration of the sugar in the pharmaceutical formulation is about 3% to about 12% (w/v). In certain embodiments, the concentration of the sugar in the pharmaceutical formulation is about 6% (w/v). In certain embodiments, the sugar is a disaccharide. In certain embodiments, the disaccharide is sucrose. In some embodiments, the concentration of the sugar alcohol in the pharmaceutical formulation is between about 0.5% to about 6% (w/v). In certain embodiments, the concentration of the sugar alcohol in the pharmaceutical formulation is about 1% (w/v). In certain embodiments, the sugar alcohol is derived from a monosaccharide. In certain embodiments, the sugar alcohol is mannitol.

In some embodiments, the concentration of the non-ionic surfactant in the pharmaceutical formulation is between about 0.005% to about 0.02% (w/v). In certain embodiments, the concentration of polysorbate 80 in the pharmaceutical formulation is about 0.01% (w/v). In certain embodiments, the non-ionic surfactant is a polysorbate. In certain embodiments, the polysorbate is polysorbate 80.

In some embodiments, the pH is between about 6.1 and about 6.9. In certain embodiments, the pH is between about 6.2 and about 6.8. In certain embodiments, the pH is between about 6.3 and about 6.7. In some embodiments, the pH is between about 6.4 and about 6.6. In certain embodiments, the pH is about 6.5.

In some embodiments, the pharmaceutical formulation further comprises water. In certain embodiments, the water is Water for Injection, USP.

In some embodiments, the pharmaceutical formulation comprises a bulk concentration of heterodimeric Fc-fused protein of about 1 g/L to about 10 g/L. In certain embodiments, the pharmaceutical formulation comprises a bulk concentration of heterodimeric Fc-fused protein of about 2 g/L to about 8 g/L. In certain embodiments, the pharmaceutical formulation comprises a bulk concentration of heterodimeric Fc-fused protein of about 4 g/L to about 6 g/L. In certain embodiments, the pharmaceutical formulation comprises a bulk concentration of heterodimeric Fc-fused protein of about 5 g/L.

In some embodiments, the pharmaceutical formulation comprises a concentration of the protein for administration of about 0.5 g/L to about 1.5 g/L. In certain embodiments, the pharmaceutical formulation comprises a concentration of the protein for administration of about 0.75 g/L to about 1.25 g/L. In certain embodiments, the pharmaceutical formulation comprises a concentration of the protein for administration of about 1 g/L.

In some embodiments, the formulation is designed to be stored at a temperature between 2° C. and 8° C. In some embodiments, the pharmaceutical formulation is a clear, colorless solution and free of visible particulates.

In some embodiments, the formulation has a thermal stability profile as defined by a Tₘ₁ of greater than about 60° C., greater than about 61° C., greater than about 62° C., greater than about 63° C., greater than about 64° C., greater than about 65° C., or greater than about 66° C.; and/or a Tₘ₂ of greater than about 70° C., greater than about 71° C., greater than about 72° C., greater than about 73° C., greater than about 74° C., greater than about 75° C., greater than about 76° C., or greater than about 77° C., as measured by differential scanning fluorimetry. In certain embodiments, the formulation has a thermal stability profile as defined by a Tₘ₁ of about 67.0° C. and a Tₘ₂ of about 77.3° C. In certain embodiments, the thermal stability profile of the pharmaceutical formulation, as defined by Tₘ₁ and/or Tₘ₂ is changed by less than about 2° C. or less than about 1° C. when the pharmaceutical formulation is incubated for 1 week at 50° C., as compared to the same pharmaceutical formulation that is incubated for 1 week at 5° C., as measured by differential scanning fluorimetry.

In some embodiments, the formulation has a thermal stability profile as defined by a T_{agg} of greater than about 60° C., greater than about 61° C., greater than about 62° C., greater than about 63° C., greater than about 64° C., greater than about 65° C., greater than about 66° C., or greater than about 67° C., as measured by differential scanning fluorimetry. In certain embodiments, the thermal stability profile of the pharmaceutical formulation, as defined by T_{agg} is changed by less than about 2° C. or less than about 1° C. when the pharmaceutical formulation is incubated for 1 week at 50° C., as compared to the same pharmaceutical formulation that is incubated for 1 week at 5° C., as measured by differential scanning fluorimetry.

In some embodiments, the pH of the pharmaceutical formulation does not change by more than about 0.2 or about 0.1 in pH value after the pharmaceutical formulation is incubated for 1 week at 5° C. In some embodiments, the pH of the pharmaceutical formulation does not change by more than about 0.2 or about 0.1 in pH value after the pharmaceutical formulation is incubated for 1 week at 50° C.

In some embodiments, the heterodimeric Fc-fused protein in the pharmaceutical formulation has a Z-average hydrodynamic diameter of less than about 15 nm, less than about 14 nm, less than about 13 nm, or less than about 12 nm, as measured by dynamic light scattering at 25° C. In certain embodiments, the heterodimeric Fc-fused protein in the pharmaceutical formulation has a Z-average hydrodynamic diameter of about 11.6 nm. In some embodiments, the heterodimeric Fc-fused protein in the pharmaceutical formulation has a Z-average hydrodynamic diameter of less than about 20 nm, less than about 19 nm, less than about 18 nm, less than about 17 nm, less than about 16 nm, or less than about 15 nm, as measured by dynamic light scattering at 25° C., after the pharmaceutical formulation is incubated for 2 weeks at 50° C. In certain embodiments, the heterodimeric Fc-fused protein in the pharmaceutical formulation has a Z-average hydrodynamic diameter of about 14.4 nm. In some embodiments, the heterodimeric Fc-fused protein in the pharmaceutical formulation has a Z-average hydrodynamic diameter of less than about 20 nm, less than about 19 nm, less than about 18 nm, less than about 17 nm, or less than about 16 nm, as measured by dynamic light scattering at 25° C., after the pharmaceutical formulation is subjected to five freeze thaw cycles. In certain embodiments, the heterodimeric Fc-fused protein in the pharmaceutical formulation has a Z-average hydrodynamic diameter of about 15.3 nm.

In some embodiments, the polydispersity index of the heterodimeric Fc-fused protein in the pharmaceutical formulation is less than about 0.30, less than about 0.29, less than about 0.28, or less than about 0.27, as measured by dynamic light scattering at 25° C. In some embodiments, the polydispersity index of the heterodimeric Fc-fused protein in the pharmaceutical formulation is about 0.26. In some embodiments, the polydispersity index of the heterodimeric Fc-fused protein in the pharmaceutical formulation is less than about 0.30, less than about 0.29, less than about 0.28, less than about 0.27, or less than about 0.26, as measured by dynamic light scattering at 25° C., after the pharmaceutical formulation is incubated for 2 weeks at 50° C. In some embodiments, the polydispersity index of the heterodimeric Fc-fused protein in the pharmaceutical formulation is about 0.25. In some embodiments, the polydispersity index of the heterodimeric Fc-fused protein in the pharmaceutical formulation is less than about 0.40, less than about 0.35, or less than about 0.34, as measured by dynamic light scattering at 25° C., after the pharmaceutical formulation is subjected to five freeze thaw cycles. In some embodiments, the polydispersity index of the heterodimeric Fc-fused protein in the pharmaceutical formulation is about 0.33.

In some embodiments, the purity profile of the pharmaceutical formulation, as measured by the area of the main peak as a percentage of total detected area in a SEC-HPLC analysis, is greater than about 90%, greater than about 91%, greater than about 92%, greater than about 93%, greater than about 94%, greater than about 95%, greater than about 96%, greater than about 97%, greater than about 98%, or greater than about 99%. In certain embodiments, the purity profile of the pharmaceutical formulation, as measured by the area of the main peak as a percentage of total detected area in a SEC-HPLC analysis, is about 99.0%.

In some embodiments, the purity profile of the pharmaceutical formulation, as measured by the area of the main peak as a percentage of total detected area in a SEC-HPLC analysis, is greater than about 75%, greater than about 80%, greater than about 81%, greater than about 82%, greater than about 83%, greater than about 84%, or greater than about 85%, after the pharmaceutical formulation is incubated for 2 weeks at 50° C. In certain embodiments, the purity profile of the pharmaceutical formulation, as measured by the area of the main peak as a percentage of total detected area in a SEC-HPLC analysis, is about 85.2%.

In some embodiments, the purity profile of the pharmaceutical formulation, as measured by the area of the main peak as a percentage of total detected area in a SEC-HPLC analysis, is greater than about 90%, greater than about 91%, greater than about 92%, greater than about 93%, greater than about 94%, greater than about 95%, greater than about 96%, greater than about 97%, or greater than about 98%, after the pharmaceutical formulation is subjected to five freeze thaw cycles. In certain embodiments, the purity profile of the pharmaceutical formulation, as measured by the area of the main peak as a percentage of total detected area in a SEC-HPLC analysis, is about 98.9%.

In another aspect, the disclosure provides for a method comprising administering to a subject in need thereof, the pharmaceutical formulation as a single-dose therapy.

In another aspect, the disclosure provides for a method comprising administering to a subject in need thereof, the pharmaceutical formulation in a multiple-dose therapy at an interval of at least three weeks between the doses or at least four weeks between the doses. In some embodiments, the pharmaceutical formulation is administered to the subject once every three weeks. In some embodiments, the pharmaceutical formulation is administered to the subject once every four weeks. In certain embodiments, the pharmaceutical formulation is administered to the subject once every six weeks.

In some embodiments, the method further comprises stopping the multi-dose therapy if the subject develops progressive disease, unacceptable toxicity, or meets a criterion for withdrawal. In some embodiments, if the subject experiences a complete response (CR) during the multi-dose therapy, then the multi-dose therapy is further administered for at least 12 months after the confirmation of the complete response. In certain embodiments, the total duration of the multi-dose therapy is equal to or less than 24 months. In certain embodiments, the total treatment duration is greater than 24 months.

In some embodiments, the pharmaceutical formulation is administered by subcutaneous injection.

In some embodiments, the pharmaceutical formulation is administered to the subject in an amount sufficient to provide the heterodimeric Fc-fused protein at a dosage of between about 0.05 μg/kg to about 1.75 μg/kg, based on the subject's weight. In some embodiments, the pharmaceutical formulation is administered to the subject in an amount sufficient to provide the heterodimeric Fc-fused protein at a dosage of about 0.05 μg/kg, about 0.10 μg/kg, about 0.20 μg/kg, about 0.40 μg/kg, about 0.60 μg/kg, about 0.80 μg/kg, about 1.00 μg/kg, about 1.20 μg/kg, about 1.40 μg/kg, or about 1.75 μg/kg, based on the subject's weight. In certain embodiments, the pharmaceutical formulation is administered to the subject in an amount sufficient to provide the heterodimeric Fc-fused protein at a dosage of greater than 0.00 μg/kg and less than about 0.05 μg/kg, based on the subject's weight. In certain embodiments, the pharmaceutical formulation is administered to the subject in an amount sufficient to provide the heterodimeric Fc-fused protein at a dosage of greater than about 1.75 μg/kg, based on the subject's weight.

In some embodiments, the subject has cancer. In certain embodiments, the subject has a locally advanced or metastatic solid tumor. In certain embodiments, the presence of the cancer in the subject is confirmed using the Response Evaluation Criteria for Solid Tumors (RECIST), version 1.1. In certain embodiments, the cancer is selected from the group consisting of: melanoma, non-small cell lung cancer (NSCLC), small cell lung cancer (SCLC), head and neck squamous cell carcinoma (HNSCC), classical Hodgkin lymphoma, primary mediastinal large B-Cell lymphoma, bladder cancer, urothelial carcinoma, micro-satellite instability high cancer, colorectal cancer, gastric cancer, oesophageal cancer, cervical cancer, hepatocellular carcinoma, Merkel cell carcinoma, renal cell carcinoma (RCC), endometrial carcinoma, cutaneous T cell lymphoma, and triple negative breast cancer. In certain embodiments, the cancer is selected from the group consisting of: melanoma, non-small cell lung cancer (NSCLC), small cell lung cancer (SCLC), head and neck squamous cell carcinoma (HNSCC), classical Hodgkin lymphoma, primary mediastinal large B-Cell lymphoma, bladder cancer, urothelial carcinoma, micro-satellite instability high cancer, colorectal cancer, gastric cancer, oesophageal cancer, cervical cancer, ovarian cancer, prostate cancer, hepatocellular carcinoma, Merkel cell carcinoma, renal cell carcinoma (RCC), endometrial carcinoma, cutaneous T cell lymphoma, and triple negative breast cancer. In certain embodiments, the subject is anti-PD-1 refractory.

In some embodiments, the subject has melanoma. In certain embodiments, the subject has previously been treated with an anti-PD-1 antibody for at least 6 weeks. In certain embodiments, the subject has been confirmed of progression of disease at least 4 weeks after the initial diagnosis of progression of disease while receiving an anti-PD-1 antibody. In certain embodiments, progression of disease is confirmed by radiological or clinical observation. In certain embodiments, if the subject has a tumor comprising a BRAF activating mutation, then the subject has previously been treated with a BRAF inhibitor.

In some embodiments, the subject has RCC. In certain embodiments, the RCC has clear cell histology. In certain embodiments, the patient has previously been treated with an anti-PD-1/PD-L1 antibody and/or an anti-vascular endothelial growth factor therapy. In certain embodiments, the subject has previously received three or fewer lines of therapy.

In some embodiments, the subject has urothelial carcinoma. In certain embodiments, the subject has locally advanced or metastatic transitional cell carcinoma of the urothelium. In certain embodiments, the subject has previously been treated with a single treatment comprising a platinum-containing regimen and has shown radiographic progression recurrence within 6 months after the last administration of the platinum-containing regimen. In certain embodiments, the subject has previously received two or less lines of therapy. In certain embodiments, the subject has not previously received a checkpoint inhibitor (e.g., anti-PD-1 or anti-PD-L1 antibody) therapy as a monotherapy or in combination with a platinum based chemotherapy.

In some embodiments, the pharmaceutical formulation is administered to the subject as a monotherapy.

In some embodiments, the pharmaceutical formulation is administered to the subject as a combination therapy.

In some embodiments, the method further comprises administering to the subject an anti-PD-1 antibody. In certain embodiments, the anti-PD-1 antibody is pembrolizumab. In certain embodiments, the pembrolizumab is administered intravenously. In certain embodiments, the pembrolizumab is administered at a dose of 200 mg. In certain embodiments, the administration of pembrolizumab precedes each administration of the pharmaceutical formulation. In certain embodiments, the pharmaceutical formulation is administered within 1 hour after completion of administration of pembrolizumab.

In certain embodiments, the anti-PD-1 antibody is nivolumab. In certain embodiments, the nivolumab is administered intravenously. In certain embodiments, the nivolumab is administered at a dose of about 480 mg. In certain embodiments, the administration of nivolumab precedes each administration of the pharmaceutical formulation. In certain embodiments, the pharmaceutical formulation is administered within 1 hour after completion of administration of nivolumab.

In some embodiments the combination therapy is for treatment of a cancer selected from the group consisting of: melanoma, NSCLC, SCLC, RCC, classical Hodgkin lymphoma, HNSCC, urothelial carcinoma, colorectal cancer, hepatocellular carcinoma, and oesophageal cancer. In some embodiments the combination therapy is for treatment of a cancer selected from the group consisting of: melanoma, NSCLC, SCLC, RCC, classical Hodgkin lymphoma, HNSCC, urothelial carcinoma, colorectal cancer, hepatocellular carcinoma, oesophageal cancer, gastric cancer, ovarian cancer, and prostate cancer. In some embodiments, the cancer is melanoma. In some embodiments, the melanoma is unresectable. In some embodiments the cancer is colorectal cancer. In some embodiments, the colorectal cancer is microsatellite instability-high (MSI-H) or mismatch repair deficient metastatic (dMMR) colorectal cancer.

In some embodiments, the method further comprises performing a surgical intervention to lyse cancer cells, remove a tumor, or debulk a tumor in the subject. In certain embodiments, the surgical intervention comprises cryotherapy. In certain embodiments, the surgical intervention comprises hyperthermic therapy. In certain embodiments, the surgical intervention comprises administering to the subject a radiotherapy. In certain embodiments, the radiotherapy is a stereotactic body radiation therapy (SBRT).

In some embodiments, the method further comprises administering to the subject an NK cell-targeting therapy. In certain embodiments, the subject is administered a multi-specific binding protein. In some embodiments, the method further comprises administering to the subject a chimeric antigen receptor therapy. In some embodiments, the method further comprises administering to the subject a cytokine therapy. In some embodiments, the method further comprises administering to the subject an innate immune system agonist therapy. In some embodiments, the method further comprises administering to the subject a chemotherapy. In some embodiments, the method further comprises administering to the subject a targeted antigen therapy. In some embodiments, the method further comprises administering to the subject an oncolytic virus therapy.

In another aspect, the disclosure provides for a method of detecting toxicity in a subject receiving a pharmaceutical formulation comprising measuring the concentration of C-reactive protein (CRP) in the subject's blood, wherein the pharmaceutical formulation comprises a heterodimeric Fc-fused protein and a pharmaceutically acceptable carrier, and wherein the heterodimeric Fc-fused protein comprises a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization. In some embodiments, if the CRP concentration in the subject's blood is higher than a threshold CRP concentration, then the subject is identified as being at risk for developing an adverse drug reaction; and if the CRP concentration in the subject's blood is about the same or lower than the threshold C-reactive protein concentration, the subject is not identified as being at risk for developing an adverse drug reaction. In certain embodiments, if the CRP concentration in the subject's blood is higher than the threshold CRP concentration, then the administration of the pharmaceutical formulation is paused, the heterodimeric Fc-fused protein is administered at a lower dose, or a remedial action is taken to reduce or alleviate the formulation's toxicity effects in the subject.

In some embodiments of the pharmaceutical formulation or the method described herein, the first and second antibody Fc domain polypeptides are human IgG1 Fc domain polypeptides. In certain embodiments, the multisubunit cytokine is a human IL12. In certain embodiments, the human IgG1 Fc domain polypeptides comprise one or more mutation(s) that reduce(s) an effector function of an Fc. In certain embodiments, the first and second antibody Fc domain polypeptides comprise mutations selected from L234A, L235A or L235E, G237A, P329A, A330S, and P331S, numbered according to the EU numbering system. In certain embodiments, the first and second antibody Fc domain polypeptides each comprise mutations L234A, L235A, and P329A. In certain embodiments, the first subunit of a multisubunit cytokine is a p40 subunit of IL12 and the second subunit of a multisubunit cytokine is a p35 subunit of IL12. In certain embodiments of the pharmaceutical formulation or the method, the first subunit of a multisubunit cytokine comprises the amino acid sequence of SEQ ID NO: 127 and the second subunit of a multisubunit cytokine comprises the amino acid sequence of SEQ ID NO: 128. In certain embodiments of the pharmaceutical formulation or the method, the second subunit of a multisubunit cytokine is fused to the second antibody Fc domain by a linker comprising the amino acid sequence of SEQ ID NO: 108. In certain embodiments of the pharmaceutical formulation or the method, the first antibody Fc domain comprises mutations L234A, L235A, P329A, Y349C, K360E, and K409W, and the second antibody Fc domain comprises mutations L234A, L235A, P329A, Q347R, S354C, D399V, and F405T. In certain embodiments of the pharmaceutical formulation or the method, the first antibody Fc domain comprises the amino acid sequence of SEQ ID NO:215, and the second antibody Fc domain comprises the amino acid sequence of SEQ ID NO:216. In certain embodiments of the pharmaceutical formulation or the method, the first antibody Fc domain peptide comprises the amino acid sequence of SEQ ID NO:290 and the second antibody Fc domain peptide comprises the amino acid sequence of SEQ ID NO:291.

In another aspect, provided herein is a kit comprising one or more vessels comprising a pharmaceutical formulation, wherein the pharmaceutical formulation comprises a heterodimeric Fc-fused protein comprising a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization; and a pharmaceutically acceptable carrier, and the one or more vessels collectively comprise about 0.1 mg-about 2 mg of heterodimeric Fc-fused protein. In certain embodiments, the one or more vessels collectively comprise about 0.5 mg to about 2 mg of heterodimeric Fc-fused protein. In certain embodiments, the one or more vessels collectively comprise about 1 mg of heterodimeric Fc-fused protein. In certain embodiments, the kit comprises one vessel comprising about 1 mg of heterodimeric Fc-fused protein. In some embodiments, the pharmaceutical formulation is a lyophilized formulation or a liquid formulation. In certain embodiments, the pharmaceutical formulation is a liquid formulation supplied in a volume of 1 mL.

In another aspect, the present disclosure provides for a use of a heterodimeric Fc-fused protein in the manufacture of a medicament for treating a cancer, wherein the medicament is manufactured in a liquid pharmaceutical formulation comprising about 0.5 g/L to about 1.5 g/L of the heterodimeric Fc-fused protein contained in one or more vessels, wherein the heterodimeric Fc-fused protein comprises a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization. In some embodiments, the liquid pharmaceutical formulation comprises about 1.0 g/L of the heterodimeric Fc-fused protein.

In another aspect, provided herein is a use of a heterodimeric Fc-fused protein in the manufacture of a medicament for treating a cancer, wherein the medicament is manufactured in a liquid pharmaceutical formulation comprising about 0.1 mg-about 2 mg of heterodimeric Fc-fused protein contained in one or more vessels, wherein the heterodimeric Fc-fused protein comprises a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization. In some embodiments, the liquid pharmaceutical formulation comprises 1 mg of heterodimeric Fc-fused protein. In some embodiments, the medicament is contained in one vessel. In some embodiments, wherein each vessel contains 1 mg of heterodimeric Fc-fused protein. In certain embodiments, the medicament is administered to the subject on day 1, every 3 weeks. In some embodiments, the medicament is administered to the subject on day 1, every 4 weeks. In some embodiments, the medicament is administered subcutaneously. In some embodiments, the medicament is administered in a volume of about 0.1 mL to about 1 mL. In certain embodiments, the medicament is administered in a volume of about 1 mL. In some embodiments, the medicament is administered to a maximum of two injection sites. In certain embodiments, a second injection is completed within 10 minutes after a first injection. In some embodiments, the medicament is administered at a dose of about 0.05 mg/kg to about 1.75 mg/kg. In certain embodiments, the medicament is administered at a dose of about 1 mg/kg. In some embodiments, the medicament is diluted prior to administration in a solution of 0.9% saline (sodium chloride for injection) and 0.01% polysorbate 80.

In another aspect, provided herein is a method of manufacturing a heterodimeric Fc-fused protein for the preparation of a pharmaceutical formulation thereof, the method comprising adding acetic acid to a solution comprising the heterodimeric Fc-fused protein obtained from a Chinese Hamster Ovary (CHO) cell culture expressing the heterodimeric Fc-fused protein for 30 minutes to 90 minutes, wherein the acetate adjusts and maintains the pH of the solution at pH 3.55 to 3.75, and wherein the heterodimeric Fc-fused protein comprises a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization. In certain embodiments, the acetic acid is added to the solution comprising the heterodimeric Fc-fused protein for about 60 minutes. In certain embodiments, the acetic acid adjusts and maintains the pH of the solution to about 3.65. In certain embodiments, the CHO cell culture expressing the heterodimeric Fc-fused protein is maintained in suspension. In certain embodiments, the CHO cell culture expressing the heterodimeric Fc-fused protein is cultured for 7-21 days in a bioreactor. In certain embodiments, the CHO cell culture expressing the heterodimeric Fc-fused protein is cultured for 14 days in a bioreactor. In some embodiments, the CHO cell culture expressing the heterodimeric Fc-fused protein is harvested by depth filtration to yield a CHO harvest medium. In certain embodiments, the depth filtration is a two-stage single-use depth filtration consisting of DOHC and XOHC filters. In certain embodiments, the heterodimeric Fc-fused protein is purified from the CHO harvest medium using Protein A capture chromatography, mixed mode chromatography, and cation exchange chromatography to yield the solution comprising the heterodimeric Fc-fused protein.

In some embodiments, the Protein A capture chromatography comprises equilibrating a Protein A resin with 20 mM Tris, 150 mM NaCl at pH 7.5, loading CHO harvest medium onto the Protein A resin; washing the loaded Protein A resin with 20 mM Tris, 150 mM NaCl at pH 7.5; washing the loaded Protein A resin with 50 mM acetate at pH 5.4; and eluting the heterodimeric Fc-fused protein from the Protein A resin with 50 mM acetate, 100 mM arginine at pH 3.7 and collecting by 280 nm UV starting at 1.25 AU/cm ascending and ending at 1.25 AU/cm descending. In certain embodiments, the acetic acid is added at a concentration of 0.5M to the solution comprising the heterodimeric Fc-fused protein eluted from the Protein A resin, wherein the acetic acid acidifies the pH of the solution to pH 3.65 for 60 minutes, followed by neutralization of the solution to pH 5.2 by adding 2M Tris. In certain embodiments, following acidification and neutralization of the solution, the solution comprising the heterodimeric Fc-fused protein is passed through a 0.2 μm filter. In certain embodiments, the filtered solution comprising the heterodimeric Fc-fused protein eluted from the Protein A resin is passed through X0SP depth filtration.

In some embodiments, mixed mode chromatography comprises equilibrating a mixed mode chromatography column with 50 mM acetate at pH 5.2; loading the solution passed through X0SP filtration onto the mixed mode chromatography column; washing the loaded mixed mode chromatography column with 50 mM acetate at pH 5.2; and eluting the heterodimeric Fc-fused protein from the mixed mode chromatography column with 50 mM Acetate, 250 mM NaCl at pH 5.2 and collecting by 280 nm UV starting at 0.625 AU/cm ascending and ending at 1.50 AU/cm descending. In certain embodiments, the solution comprising the heterodimeric Fc-fused protein eluted from the mixed mode chromatography column is passed through a 0.2 μm filter.

In some embodiments, cation exchange chromatography comprises equilibrating a cation exchange chromatography resin with 50 mM Tris at pH 7.4; loading the filtered solution eluted from the mixed mode chromatography column onto the cation exchange chromatography resin; washing the loaded cation exchange chromatography resin with 50 mM Tris at pH 7.4; and eluting the heterodimeric Fc-fused protein from the cationic exchange chromatography resin with a gradient of 50 mM Tris at pH 7.4 and 50 mM Tris, 0.5 M NaCl at pH 7.4, and collecting by 280 nm UV starting at 2.5 AU/cm ascending and ending at 4.5 AU/cm descending. In certain embodiments, the solution comprising the heterodimeric Fc-fused protein eluted from the cation exchange chromatography resin is passed through a 0.2 μm filter. In certain embodiments, the filtered solution comprising the heterodimeric Fc-fused protein eluted from the cation exchange chromatography resin is nanofiltrated through a prefilter, a 20 nm nominal filter, and a 0.2 μm membrane.

In some embodiments, the nanofiltrated solution comprising the heterodimeric Fc-fused protein is ultrafiltrated and diafiltrated, wherein ultrafilitration and diafiltration comprises equilibrating an ultrafiltration system with 50 mM Tris, 265 mM NaCl at pH 7.4; concentrating the nanofiltrated solution comprising the heterodimeric Fc-fused protein to a concentration of about 5.0 g/L; exchanging the buffer using 7 diavolumes of 20 mM citrate at pH 6.5; concentration the diafiltrated solution comprising the heterodimeric Fc-fused protein to a concentration of about 11.0 g/L; diluting the concentration solution comprising the heterodimeric Fc-fused protein to a concentration of about 5 g/L to about 10 g/L with 20 mM citrate at pH 6.5; and adding 20 mM citrate, 18% (w/v) sucrose, 3% (w/v) mannitol, 0.03% (w/v) polysorbate-80 at pH 6.5 to achieve a final concentration of the ultrafitration/diafiltration retentate solution comprising the heterodimeric Fc-fused protein of 20 mM citrate, 6% (w/v) sucrose, 1% (w/v) mannitol, 0.01% (w/v) polysorbate-80. In certain embodiments, the ultrafiltrated/diafiltrated solution comprising the heterodimeric Fc-fused protein is passed through a 0.2 μm membrane to yield a bulk drug substance.

In some embodiments, the bulk drug substance is diluted to an 80% drug product solution in a 0.2 μm filtered buffer comprising 20 mM citrate, 6% (w/v) sucrose, 1% (w/v) mannitol, and 0.01% (w/v) polysorbate-80 at pH 6.5. In certain embodiments, the bulk drug substance or 80% drug product is diluted to a concentration for administration of 1 mg/mL of the heterodimeric Fc-fused protein in a 0.2 μm filtered buffer comprising 20 mM citrate, 6% (w/v) sucrose, 1% (w/v) mannitol, and 0.01% (w/v) polysorbate-80 at pH 6.5.

A method of treating cancer in a subject who has received treatment with a checkpoint inhibitor antibody for at least 6 weeks, the method comprising administering a pharmaceutical formulation comprising a heterodimeric Fc-fused protein and a pharmaceutically acceptable carrier to the subject, wherein the heterodimeric Fc-fused protein comprises a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization. In certain embodiments, the checkpoint inhibitor antibody is an anti-programmed cell death protein 1 (PD-1) antibody. In certain embodiments, the cancer is melanoma. In certain embodiments, the melanoma is unresectable or metastatic. In certain embodiments, the subject is confirmed to have progressive disease at least 4 weeks after the initial diagnosis of progressive disease while receiving the anti-PD-1 antibody. In certain embodiments, the subject is confirmed to have progressive disease at least 4 weeks after the initial diagnosis of progressive disease while receiving the anti-PD-1 antibody. In certain embodiments, the progressive disease is confirmed by radiological or clinical observation.

In another aspect, provided herein is a method of treating cancer in a subject who has received treatment with a checkpoint inhibitor antibody or an anti-vascular endothelial growth factor therapy as a monotherapy, or in combination, the method comprising administering a pharmaceutical formulation comprising a heterodimeric Fc-fused protein and a pharmaceutically acceptable carrier to the subject, wherein the heterodimeric Fc-fused protein comprises a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization. In certain embodiments, the checkpoint inhibitor antibody is an anti-PD-1 antibody or an anti-PD-L1 antibody.

In certain embodiments, the cancer is advanced renal cell carcinoma (RCC). In certain embodiments, the RCC is unresectable or metastatic. In certain embodiments, the RCC has a clear cell component. In certain embodiments, the subject received no more than 3 previous lines of therapy. In certain embodiments, the subject has not received treatment with a checkpoint inhibitor. In certain embodiments, the checkpoint inhibitor comprises an anti-PD-1 antibody or anti-PD-L1 antibody as a monotherapy or in combination with a platinum based chemotherapy.

In another aspect, provided herein is a method of treating cancer in a subject who has received treatment with only one platinum-containing regimen, the method comprising administering a pharmaceutical formulation comprising a heterodimeric Fc-fused protein and a pharmaceutically acceptable carrier to the subject, wherein the heterodimeric Fc-fused protein comprises a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization. In certain embodiments, the platinum containing regimen is platinum in combination with an agent selected from gemcitabine, methotrexate, vinblastine, and doxorubicin. In certain embodiments, the cancer is locally advanced or metastatic transitional cell urothelial carcinoma. In certain embodiments, the urothelial carcinoma includes one or more of the group consisting of the renal pelvis, ureters, urinary urothelium, and urethra. In certain embodiments, the urothelial carcinoma is inoperable. In certain embodiments, the urothelial carcinoma is characterized with radiographic progression or with recurrence within 6 months after the last administration of a platinum-containing regimen as an adjuvant.

In some embodiments, the urothelial carcinoma is considered failure of a first-line, platinum-containing regimen. In certain embodiments, the subject has received no more than 2 lines of therapy (including the platinum-containing regimen) for the treatment of the urothelial carcinoma prior to administration of the pharmaceutical formulation. In certain embodiments, the subject has not received treatment with a checkpoint inhibitor (CPI) as a first-line therapy. In certain embodiments, the checkpoint inhibitor is an anti-PD-1 antibody or anti-PD-L1 antibody. In certain embodiments, the checkpoint inhibitor is a monotherapy or in combination with a platinum based chemotherapy.

In some embodiments, the pharmaceutical formulation is administered in combination with pembrolizumab. In certain embodiments, pembrolizumab is administered once every 3 weeks. In certain embodiments, pembrolizumab is administered before administration of the pharmaceutical formulation. In certain embodiments, the pharmaceutical formulation is administered within one hour after the completion of administration of pembrolizumab. In some embodiments, pembrolizumab is administered at a dose of 200 mg. In some embodiments, pembrolizumab is administered intravenously. In some embodiments, the combination is for treatment of a cancer selected from the group consisting of: melanoma, non-small cell lung cancer (NSCLC), small cell lung cancer (SCLC), head and neck squamous cell carcinoma (HNSCC), classical Hodgkin lymphoma, primary mediastinal large B-cell lymphoma, urothelial carcinoma, microsatellite instability-high cancer, gastric cancer, oesophageal cancer, cervical cancer, hepatocellular carcinoma, Merkel cell carcinoma, renal cell carcinoma, and endometrial carcinoma. In some embodiments, the combination is for treatment of a cancer selected from the group consisting of: melanoma, non-small cell lung cancer (NSCLC), small cell lung cancer (SCLC), head and neck squamous cell carcinoma (HNSCC), classical Hodgkin lymphoma, primary mediastinal large B-cell lymphoma, urothelial carcinoma, microsatellite instability-high cancer, gastric cancer, oesophageal cancer, cervical cancer, ovarian cancer, prostate cancer, hepatocellular carcinoma, Merkel cell carcinoma, renal cell carcinoma, and endometrial carcinoma.

In some embodiments, the pharmaceutical formulation is administered in combination with nivolumab. In certain embodiments, nivolumab is administered once every 4 weeks. In some embodiments, the nivolumab is administered before administration of the pharmaceutical formulation. In some embodiments, the pharmaceutical formulation is administered within one hour after the completion of administration of nivolumab. In some embodiments, the nivolumab is administered at a dose of about 480 mg. In some embodiments, the nivolumab is administered intravenously. In some embodiments the combination is for treatment of a cancer selected from the group consisting of: melanoma, non-small cell lung cancer (NSCLC), small cell lung cancer (SCLC), renal cell carcinoma, classical Hodgkin lymphoma, head and neck squamous cell carcinoma (HNSCC), colorectal cancer, hepatocellular carcinoma, bladder cancer, and oesophageal cancer. In some embodiments the combination is for treatment of a cancer selected from the group consisting of: melanoma, non-small cell lung cancer (NSCLC), small cell lung cancer (SCLC), renal cell carcinoma, classical Hodgkin lymphoma, head and neck squamous cell carcinoma (HNSCC), colorectal cancer, hepatocellular carcinoma, bladder cancer, oesophageal cancer, gastric cancer, ovarian cancer, and prostate cancer. In some embodiments, the cancer is melanoma. In certain embodiments, the melanoma is unresectable or metastatic. In some embodiments the cancer is colorectal cancer. In certain embodiments, the colorectal cancer is microsatellite instability-high (MSI-H) or mismatch repair deficient (dMMR) metastatic colorectal cancer.

In some embodiments, the pharmaceutical formulation is administered to the subject on day 1, every 3 weeks. In some embodiments, the pharmaceutical formulation is administered to the subject on day 1, every 4 weeks. In some embodiments, the pharmaceutical formulation is administered subcutaneously. In some embodiments, the pharmaceutical formulation is administered in a volume of about 0.1 mL to about 1 mL. In some embodiments, the pharmaceutical formulation is administered in a volume of about 1 mL. In some embodiments, the pharmaceutical formulation is administered to a maximum of two injection sites. In certain embodiments, a second injection is completed within 10 minutes after a first injection.

In some embodiments, the pharmaceutical formulation is administered at a dose of about 0.05 mg/kg to about 1.75 mg/kg. In certain embodiments, the pharmaceutical formulation is administered at a dose of about 1 mg/kg. In some embodiments, the pharmaceutical formulation is diluted prior to administration in a solution of 0.9% saline (sodium chloride for injection) and 0.01% polysorbate 80.

In some embodiments, the presence of the cancer is determined using the Response Evaluation Criteria for Solid Tumors (RECIST), version 1.1. In some embodiments, a subject who has a confirmed complete response is treated with the pharmaceutical formulation for at least 12 months after confirmation unless a criterion for discontinuation is met.

In another aspect, provided herein is a method of treating a subject whose blood concentration of C-reactive protein (CRP) is monitored, the method comprising administering to the subject a pharmaceutical formulation comprising a heterodimeric Fc-fused protein and a pharmaceutically acceptable carrier, wherein the heterodimeric Fc-fused protein comprises a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization. In some embodiments, if the CRP concentration in the subject's blood is higher than a threshold CRP concentration, then the subject is identified as being at risk for developing an adverse drug reaction; and if the CRP concentration in the subject's blood is about the same or lower than the threshold C-reactive protein concentration, the subject is not identified as being at risk for developing an adverse drug reaction. In some embodiments, if the CRP concentration in the subject's blood is higher than the threshold CRP concentration, then (1) the administration of the pharmaceutical formulation is paused; (2) the heterodimeric Fc-fused protein is administered at a lower dose; or (3) a remedial action is taken to reduce or alleviate the formulation's toxicity effects in the subject.

In another aspect, provided herein is a method of treating cancer in a subject in need thereof, the method comprising subcutaneous administration of a pharmaceutical formulation comprising a heterodimeric Fc-fused protein and pharmaceutically acceptable carrier to the subject, wherein the heterodimeric Fc-fused protein comprises a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization; and the pharmaceutical formulation comprises citrate; a sugar; a sugar alcohol; and a non-ionic surfactant, and the pH of the formulation is between 5.5 and 7.0.

In some embodiments of the kit, the use, or the method provided herein, the first Fc region and second Fc region of the heterodimeric Fc-fused protein are human IgG1 Fc regions. In some embodiments, the human IgG1 Fc regions comprise one or more mutation(s) that reduce(s) an effector function of an Fc. In some embodiments of the kit, the use, or the method provided herein, the first Fc region and second Fc region comprise one or more mutation(s) selected from L234A, L235A or L235E, G237A, P329A, A330S, and P331S, numbered according to the EU numbering system. In some embodiments, the first Fc region and second Fc region each comprise mutations L234A, L235A, and P329A.

In certain embodiments of the kit, the use, or the method provided herein, the p40 subunit of IL12 comprises the amino acid sequence of SEQ ID NO: 127 and the p35 subunit of IL-12 comprises the amino acid sequence of SEQ ID NO: 128. In certain embodiments of the kit, the use, or the method provided herein, the p35 subunit of IL-12 is fused to the second Fc region by a linker comprising the amino acid sequence of SEQ ID NO: 108. In some embodiments, the first Fc region comprises mutations L234A, L235A, P329A, Y349C, K360E, and K409W, and the second Fc region comprises mutations L234A, L235A, P329A, Q347R, S354C, D399V, and F405T. In certain embodiments, the first Fc region comprises the amino acid sequence of SEQ ID NO:215, and the second Fc region comprises the amino acid sequence of SEQ ID NO:216. In some embodiments, the first Fc region linked to the p40 subunit of IL12 comprises the amino acid sequence of SEQ ID NO:290 and the second Fc region linked to the p35 subunit of IL-12 comprises the amino acid sequence of SEQ ID NO:291.

In some embodiments of the kit, the use, or the method provided herein, the pharmaceutical formulation comprises: (a) citrate; (b) a sugar; (c) a sugar alcohol; and (d) a non-ionic surfactant, further wherein the pH of the formulation is between about 6.0 and about 7.0. In some embodiments of the kit, the use, or the method provided herein, the concentration of citrate in the pharmaceutical formulation is about 10 to about 30 mM. In some embodiments, the concentration of citrate in the pharmaceutical formulation is about 20 mM. In some embodiments, the concentration of the sugar in the pharmaceutical formulation is about 3% to about 12% (w/v). In some embodiments, the concentration of the sugar in the pharmaceutical formulation is about 6% (w/v). In some embodiments, the sugar is a disaccharide. In some embodiments, the disaccharide is sucrose. In some embodiments, the concentration of the sugar alcohol in the pharmaceutical formulation is between about 0.5% to about 6% (w/v). In some embodiments, the concentration of the sugar alcohol in the pharmaceutical formulation is about 1% (w/v). In some embodiments, the sugar alcohol is derived from a monosaccharide. In some embodiments, the sugar alcohol is mannitol.

In some embodiments of the kit, the use, or the method provided herein, the concentration of the non-ionic surfactant in the pharmaceutical formulation is between about 0.005% to about 0.02% (w/v). In some embodiments of the kit, the use, or the method provided herein, the concentration of polysorbate 80 in the pharmaceutical formulation is about 0.01% (w/v). In some embodiments, the non-ionic surfactant is a polysorbate. In some embodiments of the kit, the use, or the method provided herein, the polysorbate is polysorbate 80. In some embodiments, the pH is between about 6.1 and about 6.9. In some embodiments, the pH is between about 6.2 and about 6.8. In some embodiments, the pH is between about 6.3 and about 6.7. In some embodiments, the pH is between about 6.4 and about 6.6. In some embodiments of the kit, the use, or the method provided herein, the pH is about 6.5.

In some embodiments of the kit, the use, or the method provided herein, the pharmaceutical formulation further comprises water. In some embodiments of the kit, the use, or the method provided herein, the water is Water for Injection, USP.

In some embodiments of the kit, the use, or the method provided herein, the pharmaceutical formulation comprises a bulk concentration of heterodimeric Fc-fused protein of about 1 g/L to about 10 g/L. In some embodiments, the pharmaceutical formulation comprises a bulk concentration of heterodimeric Fc-fused protein of about 2 g/L to about 8 g/L. In some embodiments, the pharmaceutical formulation comprises a bulk concentration of heterodimeric Fc-fused protein of about 4 g/L to about 6 g/L. In some embodiments, the pharmaceutical formulation comprises a bulk concentration of heterodimeric Fc-fused protein of about 5 g/L. In some embodiments, the pharmaceutical formulation comprises a concentration of the protein for administration of about 0.5 g/L to about 1.5 g/L. In some embodiments, the pharmaceutical formulation comprises a concentration of the protein for administration of about 0.75 g/L to about 1.25 g/L. In some embodiments, the pharmaceutical formulation comprises a concentration of the protein for administration of about 1 g/L.

In some embodiments of the kit, the use, or the method provided herein, the pharmaceutical formulation is designed to be stored at a temperature between 2° C. and 8° C. In some embodiments, the pharmaceutical formulation is a clear, colorless solution and free of visible particulates.

In some embodiments of the kit, the use, or the method provided herein, the pharmaceutical formulation has a thermal stability profile as defined by a Tₘ₁ of greater than about 60° C., greater than about 61° C., greater than about 62° C., greater than about 63° C., greater than about 64° C., greater than about 65° C., or greater than about 66° C.; and/or a Tₘ₂ of greater than about 70° C., greater than about 71° C., greater than about 72° C., greater than about 73° C., greater than about 74° C., greater than about 75° C., greater than about 76° C., or greater than about 77° C., as measured by differential scanning fluorimetry. In some embodiments, the formulation has a thermal stability profile as defined by a Tₘ₁ of about 67.0° C. and a Tₘ₂ of about 77.3° C.

In some embodiments of the kit, the use, or the method provided herein, the thermal stability profile of the pharmaceutical formulation, as defined by Tₘ₁ and/or Tₘ₂ is changed by less than about 2° C. or less than about 1° C. when the pharmaceutical formulation is incubated for 1 week at 50° C., as compared to the same pharmaceutical formulation that is incubated for 1 week at 5° C., as measured by differential scanning fluorimetry. In some embodiments, the formulation has a thermal stability profile as defined by a T_{agg} of greater than 60° C., greater than about 61° C., greater than about 62° C., greater than about 63° C., greater than about 64° C., greater than about 65° C., greater than about 66° C., or greater than about 67° C., as measured by differential scanning fluorimetry. In some embodiments, the thermal stability profile of the pharmaceutical formulation, as defined by T_{agg} is changed by less than about 2° C. or less than about 1° C. when the pharmaceutical formulation is incubated for 1 week at 50° C., as compared to the same pharmaceutical formulation that is incubated for 1 week at 5° C., as measured by differential scanning fluorimetry.

In some embodiments of the kit, the use, or the method provided herein, the pH of the pharmaceutical formulation does not change by more than about 0.2 or about 0.1 in pH value after the pharmaceutical formulation is incubated for 1 week at 5° C. In some embodiments, the pH of the pharmaceutical formulation does not change by more than about 0.2 or about 0.1 in pH value after the pharmaceutical formulation is incubated for 1 week at 50° C.

In some embodiments of the kit, the use, or the method provided herein, the heterodimeric Fc-fused protein in the pharmaceutical formulation has a Z-average hydrodynamic diameter of less than about 15 nm, less than about 14 nm, less than about 13 nm, or less than about 12 nm, as measured by dynamic light scattering at 25° C. In some embodiments, the heterodimeric Fc-fused protein in the pharmaceutical formulation has a Z-average hydrodynamic diameter of about 11.6 nm.

In some embodiments of the kit, the use, or the method provided herein, the heterodimeric Fc-fused protein in the pharmaceutical formulation has a Z-average hydrodynamic diameter of less than about 20 nm, less than about 19 nm, less than about 18 nm, less than about 17 nm, less than about 16 nm, or less than about 15 nm, as measured by dynamic light scattering at 25° C., after the pharmaceutical formulation is incubated for 2 weeks at 50° C. In some embodiments, the heterodimeric Fc-fused protein in the pharmaceutical formulation has a Z-average hydrodynamic diameter of about 14.4 nm.

In some embodiments of the kit, the use, or the method provided herein, the heterodimeric Fc-fused protein in the pharmaceutical formulation has a Z-average hydrodynamic diameter of less than about 20 nm, less than about 19 nm, less than about 18 nm, less than about 17 nm, or less than about 16 nm, as measured by dynamic light scattering at 25° C., after the pharmaceutical formulation is subjected to five freeze thaw cycles. In some embodiments, the heterodimeric Fc-fused protein in the pharmaceutical formulation has a Z-average hydrodynamic diameter of about 15.3 nm.

In some embodiments of the kit, the use, or the method provided herein, the polydispersity index of the heterodimeric Fc-fused protein in the pharmaceutical formulation is less than about 0.30, less than about 0.29, less than about 0.28, or less than about 0.27, as measured by dynamic light scattering at 25° C. In some embodiments, the polydispersity index of the heterodimeric Fc-fused protein in the pharmaceutical formulation is about 0.26.

In some embodiments of the kit, the use, or the method provided herein, the polydispersity index of the heterodimeric Fc-fused protein in the pharmaceutical formulation is less than about 0.30, less than about 0.29, less than about 0.28, less than about 0.27, or less than about 0.26, as measured by dynamic light scattering at 25° C., after the pharmaceutical formulation is incubated for 2 weeks at 50° C. In some embodiments, the polydispersity index of the heterodimeric Fc-fused protein in the pharmaceutical formulation is about 0.25.

In some embodiments of the kit, the use, or the method provided herein, the polydispersity index of the heterodimeric Fc-fused protein in the pharmaceutical formulation is less than about 0.40, less than about 0.35, or less than about 0.34, as measured by dynamic light scattering at 25° C., after the pharmaceutical formulation is subjected to five freeze thaw cycles. In some embodiments, the polydispersity index of the heterodimeric Fc-fused protein in the pharmaceutical formulation is about 0.33.

In some embodiments of the kit, the use, or the method provided herein, the purity profile of the pharmaceutical formulation, as measured by the area of the main peak as a percentage of total detected area in a SEC-HPLC analysis, is greater than about 90%, greater than about 91%, greater than about 92%, greater than about 93%, greater than about 94%, greater than about 95%, greater than about 96%, greater than about 97%, greater than about 98%, or greater than about 99%. In some embodiments, the purity profile of the pharmaceutical formulation, as measured by the area of the main peak as a percentage of total detected area in a SEC-THPLC analysis, is about 99.0%. In some embodiments, the purity profile of the pharmaceutical formulation, as measured by the area of the main peak as a percentage of total detected area in a SEC-THPLC analysis, is greater than about 75%, greater than about 80%, greater than about 81%, greater than about 82%, greater than about 83%, greater than about 84%, or greater than about 85%, after the pharmaceutical formulation is incubated for 2 weeks at 50° C. In some embodiments, the purity profile of the pharmaceutical formulation, as measured by the area of the main peak as a percentage of total detected area in a SEC-HPLC analysis, is about 85.2%.

In some embodiments of the kit, the use, or the method provided herein, the purity profile of the pharmaceutical formulation, as measured by the area of the main peak as a percentage of total detected area in a SEC-HPLC analysis, is greater than about 90%, greater than about 91%, greater than about 92%, greater than about 93%, greater than about 94%, greater than about 95%, greater than about 96%, greater than about 97%, or greater than about 98%, after the pharmaceutical formulation is subjected to five freeze thaw cycles. In some embodiments, wherein the purity profile of the pharmaceutical formulation, as measured by the area of the main peak as a percentage of total detected area in a SEC-HPLC analysis, is about 98.9%.

In summary, the present invention provides heterodimeric Fc-fused protein constructs of multisubunit proteins. These fusion protein constructs can exhibit a higher serum half-life compared to a native/natural multisubunit protein, improved yield during production, enhanced stability during storage, and/or improved efficacy when used as a therapeutic.

### BRIEF DESCRIPTION OF THE DRAWINGS

FIGS. 1A-1D illustrate various features of an exemplary heterodimeric Fc-fused protein comprising a first subunit of a multisubunit protein connected by a linker to a first antibody Fc domain polypeptide, and a second, different subunit of a multisubunit protein connected by another linker to a second antibody Fc domain polypeptide, in which the subunits are connected by two disulfide bonds. FIG. 1A shows a general schematic diagram showing the different components of an exemplary heterodimeric Fc-fused protein. FIG. 1B shows an exemplary heterodimeric Fc-fused protein which includes IL12 subunits p40 and p35, linkers, and Fc domains with mutations. FIG. 1C shows a schematic diagram illustrating exemplary mutations that can be present in the heterodimeric Fc-fused protein of FIG. 1A or FIG. 1B. FIG. 1D shows a schematic diagram illustrating exemplary disulfide bonds that can form in the heterodimeric Fc-fused protein of FIG. 1A, FIG. 1B, or FIG. 1C.

FIGS. 2A-2C are graphs showing tumor growth curves of individual mice inoculated with CT26 tumor cells and treated with recombinant mouse IL-12 (rmIL-12) (FIG. 2A), DF-mIL-12-Fc wt (FIG. 2B), DF-mIL-12-Fc si (FIG. 2C), or mIgG2a isotype control once a week.

FIG. 3 is a graph showing Kaplan-Meier survival curves of mice inoculated with CT26 tumor cells and treated with rmIL-12, DF-mIL-12-Fc wt, DF-mIL-12-Fc si, or mIgG2a isotype control once a week.

FIGS. 4A-4D are graphs showing tumor growth curves of individual mice inoculated with CT26 tumor cells and treated with DF-mIL-12-Fc wt at a molar equivalent of 1 μg rmIL-12 (FIG. 4A), DF-mIL-12-Fc si at a molar equivalent of 1 μg rmIL-12 (FIG. 4B), DF-mIL-12-Fc wt at a molar equivalent of 0.1 μg rmIL-12 (FIG. 4C), DF-mIL-12-Fc si at a molar equivalent of 0.1 μg rmIL-12 (FIG. 4D), or mIgG2a isotype control once a week.

FIG. 5 is a graph showing Kaplan-Meier survival curves of mice inoculated with CT26 tumor cells and treated with DF-mIL-12-Fc wt at a molar equivalent of 1 μg rmIL-12, DF-mIL-12-Fc si at a molar equivalent of 1 μg rmIL-12, DF-mIL-12-Fc wt at a molar equivalent of 0.1 μg rmIL-12, DF-mIL-12-Fc si at a molar equivalent of 0.1 μg rmIL-12, or mIgG2a isotype control once a week.

FIGS. 6A-6C are graphs showing tumor growth curves of individual mice inoculated with B16F10 melanoma cells and treated with rmIL-12 (FIG. 6A), DF-mIL-12-Fc wt (FIG. 6B), DF-mIL-12-Fc si (FIG. 6C), or mIgG2a isotype control once a week.

FIG. 7 is a graph showing Kaplan-Meier survival curves of mice inoculated with B16F10 melanoma cells and treated with rmIL-12, DF-mIL-12-Fc wt, DF-mIL-12-Fc si, or mIgG2a isotype control once a week.

FIGS. 8A-8D are graphs showing tumor growth curves of individual mice inoculated with B16F10 melanoma cells and treated with DF-mIL-12-Fc wt at a molar equivalent of 0.5 μg rmIL-12 (FIG. 8A), DF-mIL-12-Fc si at a molar equivalent of 0.5 μg rmIL-12 (FIG. 8B), DF-mIL-12-Fc wt at a molar equivalent of 0.1 μg rmIL-12 (FIG. 8C), DF-mIL-12-Fc si at a molar equivalent of 0.1 μg rmIL-12 (FIG. 8D), or mIgG2a isotype control once a week.

FIG. 9 is a graph showing Kaplan-Meier survival curves of mice inoculated with B16F10 melanoma cells and treated with DF-mIL-12-Fc wt at a molar equivalent of 0.5 μg rmIL-12, DF-mIL-12-Fc si at a molar equivalent of 0.5 μg rmIL-12, DF-mIL-12-Fc wt at a molar equivalent of 0.1 μg rmIL-12, DF-mIL-12-Fc si at a molar equivalent of 0.1 μg rmIL-12, or mIgG2a isotype control once a week.

FIG. 10A is a graph showing IL-12 response to treatment with DF-hIL-12-Fc si (DF IL-12-Fc) or recombinant human IL-12 (rhIL-12) using a HEK-Blue IL-12 reporter assay.

FIG. 10B is a graph showing IFNγ production by peripheral blood mononuclear cells (PBMCs) in response to treatment with DF-hIL-12-Fc si (DF IL-12-Fc) and rhIL-12.

FIG. 11 is a graph showing the relative plasma concentrations of DF-hIL-12-Fc si, rhIL-12, and IFNγ in cynomolgus monkey K2 EDTA plasma following a single intravenous dose of equimolar amounts of DF-hIL-12-Fc si or wild type rhIL-12 at 10 μg/kg.

FIGS. 12A-12B are graphs showing the PK/PD profile of rmIL-12 (FIG. 12A) and DF-mIL-12-Fc si (FIG. 12B) in naïve Balb/c mice. FIG. 12A shows the PK/PD profile of rmIL-12 in naïve Balb/c mice and FIG. 12B shows the PK/PD profile of DF-mIL-12-Fc si in naïve Balb/c miceIL-12. IL-12 and IFNγ levels in serum were analyzed by ELISA. FIG. 12C is a graph showing the PK/PD profile of DF-mIL-12-Fc si administered intravenously in naïve Balb/c mice. FIG. 12D is a graph showing the PK/PD profile of DF-mIL-12-Fc si administered intraperitoneally in naïve Balb/c mice. FIG. 12E is a graph showing the PK/PD profile of DF-mIL-12-Fc si administered subcutaneously in naïve Balb/c mice. Average serum levels represent the mean±SEM.

FIGS. 13A-13C are graphs showing tumor growth curves of B16F10 tumor-bearing mice treated with DF-mIL-12-Fc si, anti-PD-1, or a combination thereof. Mice were treated intraperitoneally with 0.5 μg isotype control or 0.5 μg DF-mIL-12-Fc si (FIG. 13A), isotype control or anti-PD-1 (FIG. 13B), and isotype control or DF-mIL-12-Fc si/anti-PD-1 (FIG. 13C). Animals were injected once a week with DF-mIL-12-Fc si and twice weekly with anti-PD-1 as indicated above. Tumor growth was assessed for 60 days. Graphs show tumor growth curves of individual mice.

FIGS. 14A-14B are graphs showing survival and body weights of B16F10 tumor-bearing mice treated DF-mIL-12-Fc si, anti-PD-1, or a combination thereof. Mice were treated with isotype, DF-mIL-12-Fc si, anti-PD-1 or in combination of DF-mIL-12-Fc si and anti-PD-1. Animals were injected once a week with 0.5 μg DF-mIL-12-Fc si and twice weekly with 200 μg anti-PD-1 or isotype. FIG. 14A shows Kaplan-Meier survival curves. FIG. 14B shows body weights of mice as averages±standard deviation.

FIGS. 15A-15C are graphs showing tumor growth curves of B16F10 tumor-bearing mice treated DF-mIL-12-Fc si, mcFAE-C26.99 TriNKETs, or a combination thereof. Mice were treated intraperitoneally with 150 μg isotype control or 0.5 μg DF-mIL-12-Fc si (FIG. 15A), isotype control or 150 μg TriNKET (FIG. 15B), and isotype control or DF-mIL-12-Fc si/TriNKET (FIG. 15C). Animals were injected once a week with DF-mIL-12-Fc si and thrice weekly with TriNKET as indicated above. Tumor growth was assessed for 72 days. Graphs show tumor growth curves of individual mice.

FIGS. 16A-16B are graphs showing survival and body weights of B16F10 tumor-bearing mice treated with DF-mIL-12-Fc si, mcFAE-C26.99 TriNKETs, or a combination thereof. Mice were treated with isotype, DF-mIL-12-Fc si, TriNKET, or a combination of DF-mIL-12-Fc si and TriNKET. Animals were injected once a week with 0.5 μg DF-mIL-12-Fc si and thrice weekly with 150 μg TriNKET or isotype. FIG. 16A shows Kaplan-Meier survival curves. FIG. 16B shows body weights of mice as averages+standard deviation.

FIG. 17 is a graph showing tumor growth curves of complete responder (CR) mice from the B16F10 tumor model experiment of FIG. 15 treated with DF-mIL-12-Fc si/TriNKET combination therapy (n=3), re-challenged by engraftment with 2×10⁵ B16F10 melanoma cells.

FIG. 18A is a graph showing tumor growth curves of individual mice inoculated with CT26 tumor cells and administered a single dose of DF-mIL-12-Fc si or mIgG2a isotype.

FIG. 18B is a graph showing body weights±standard deviation of mice inoculated with CT26 tumor cells and administered a weekly dose of DF-mIL-12-Fc si, mIgG2a isotype, or rmIL-12.

FIG. 18C is a graph showing tumor growth curves of re-challenged individual mice that were either naïve or complete responders (CR) when previously administered a single dose of DF-mIL-12-Fc si in a CT26 tumor model.

FIGS. 19A-19B are graphs showing tumor growth curves of individual mice inoculated with CT26 tumor cells and administered a weekly dose of DF-mIL-12-Fc si or mIgG2a isotype either intraperitoneally (IP)(FIG. 19A) or subcutaneously (SC) (FIG. 19B).

FIG. 20 is a graph showing tumor growth curves of individual mice inoculated with B16F10 melanoma cells and administered a single dose of DF-mIL-12-Fc si or mIgG2a isotype.

FIGS. 21A-21B are graphs showing tumor growth curves of individual mice inoculated with B16F10 melanoma cells and administered a weekly dose of DF-mIL-12-Fc si or mIgG2a isotype either intraperitoneally (IP) (FIG. 21A) or subcutaneously (SC) (FIG. 21B).

FIGS. 22A-22B are graphs showing tumor growth curves of individual mice inoculated with CT26 tumor cells and administered a single dose (FIG. 22A) or once weekly dose (FIG. 22B) of DF-mIL-12-Fc si or mIgG2A isotype intraperitoneally at a molar equivalent of 1 μg of rmIL-12.

FIGS. 23A-23B are graphs showing tumor growth curves of individual mice inoculated with CT26 tumor cells and administered a once weekly dose of DF-mIL-12-Fc si subcutaneously. FIG. 23A is a graph showing tumor growth curves of individual mice inoculated with CT26-Tyrp1 tumor cells and treated once (weekly) with either 2 μg mIgG2a isotype control or 1 μg DF-mIL-12-Fc si. FIG. 23B is a graph showing tumor growth curves of individual mice inoculated with CT26-Tyrp1 tumor cells and treated once (weekly) with either 2 μg mIgG2a isotype control or 2 μg DF-mIL-12-Fc si.

FIGS. 24A-24C are graphs showing IFNγ (FIG. 24A), CXCL9 (FIG. 24B), and CXCL10 (FIG. 24C) levels in blood (left) and tumor (right) samples at 72 hours following a single dose of DF-mIL-12-Fc si in C57BL/6 mice bearing B16F10 tumors.

FIGS. 25A-25C are line graphs showing pharmacokinetics of DF-hIL-12-Fc si in cynomolgus monkeys treated with a single subcutaneous dose of 1 μg/kg (FIG. 25A), 2 μg/kg (FIG. 25B), or 4 μg/kg (FIG. 25C) of DF-hIL-12-Fc si. 2240, 2241, 2740, 2741 (FIG. 25A); 3240, 3241, 3740, 3741 (FIG. 25B); 4240, 4241, 4740, 4741 (FIG. 25C) denote individual cynomolgus monkey subjects.

FIGS. 26A-26F are line graphs showing concentrations of IFNγ and IP10/CXCL10 in cynomolgus monkeys treated with a single subcutaneous dose of DF-hIL-12-Fc si. FIGS. 26A, 26C and 26E show IFNγ concentrations/levels of expression in cynomolgus monkeys treated with 1 μg/kg, 2 μg/kg, and 4 μg/kg of DF-hIL-12-Fc si, respectively. FIGS. 26B, 26D and 26F show IP10/CXCL10 concentrations/levels of expression in cynomolgus monkeys treated with 1 μg/kg, 2 μg/kg, and 4 μg/kg of DF-hIL-12-Fc si, respectively. 1240, 1740, 2240, 2241, 2740, 2741 (FIGS. 26A-26B); 1240, 1740, 3240, 3241, 3740, 3741 (FIGS. 26C, 26D, 26F); 1240, 1740, 4240, 4241, 4740, 4741 (FIG. 26E) denote individual cynomolgus monkey subjects.

FIG. 27 is a graph showing tumor growth curves of individual mice inoculated with breast cancer cells and administered a weekly dose of a monotherapy (isotype control, DF-mIL-12-Fc si, Doxil® (chemotherapy), or irradiated with 10 Gy) or combination therapy (DF-mIL-12-Fc si in combination with Doxil® or radiation).

FIG. 28A is a graph showing tumor growth curves of individual mice inoculated with CT26-Tyrp1 tumor cells and treated (bi-weekly) either with isotype control or anti-PD-1 antibody.

FIG. 28B is a graph showing tumor growth curve of Balb/c mice inoculated with CT26-Tyrp1 tumor cells and treated (bi-weekly) either with isotype control or anti-PD-1 antibody along with weekly treatment of 1 μg of DF-mIL-12-Fc si.

FIG. 29A is a graph showing tumor growth curves of treated (Tr) tumors in individual mice inoculated with CT26-Tyrp1 tumor cells and intratumorally treated once (weekly) with either isotype control or DF-mIL-12-Fc si. FIG. 29B is a graph showing tumor growth curves of non-treated (NT) CT26-Tyrp1 tumors in the individual mice described in FIG. 29A.

FIG. 30A is a graph showing tumor growth curves of individual mice inoculated with CT26-Tyrp1 tumor cells and treated once with either 2 μg mIgG2a isotype control or 2 μg DF-mIL-12-Fc si. FIG. 30B is a graph showing average tumor growth curves of individual mice inoculated with CT26-Tyrp1 tumor cells and treated with 2 μg mIgG2a isotype control, 1 μg DF-mIL-12-Fc si (weekly administration), 2 μg DF-mIL-12-Fc si (weekly administration), or 2 μg DF-mIL-12-Fc si (once).

FIG. 31A is a graph showing IFNγ production of PHA-stimulated PBMCs treated with DF hIL-12-Fc-si having L234A, L235A, and P329A mutations (LALAPA), or L234A, L235A, and P329G mutations (LALAPG). FIG. 31B shows flow cytometry histograms of fluorophore-conjugated hIgG1 binding to THP-1 cells in the presence or absence of DF hIL-12-Fc-si having LALAPA mutations, or LALAPG mutations.

FIG. 32 is a process flow diagram showing steps for preparing DF hIL-12-Fc si.

FIG. 33 is a process flow diagram showing steps for preparing a pharmaceutical formulation containing DF-hIL-12-Fc si.

FIG. 34A is a graph showing Ultraviolet-visible spectroscopy (UV-Vis) calculated concentrations of DF-hIL-12-Fc si in various pharmaceutical formulations containing different buffers. FIG. 34B is a graph showing pH of various pharmaceutical formulations containing DF-IL-12-Fc si.

FIGS. 35A-35B are photographs of visual appearances of the various formulations of certain pharmaceutical formulations containing DF-hIL-12-Fc si after a 1 week incubation at 5° C. (FIG. 35A) and 50° C. (FIG. 35B).

FIGS. 36A and 36B are graphs showing the average Tₘ₁ (FIG. 36A) and Tₘ₂ (FIG. 36B) of DF-hIL-12-Fc si in various pharmaceutical formulations after a 1 week incubation at 5° C.

FIGS. 36C and 36D are graphs showing the average Tₘ₁ (FIG. 36C) and Tₘ₂ (FIG. 36D) of DF-hIL-12-Fc si in various pharmaceutical formulations after a 1 week incubation at 50° C.

FIGS. 36E-36H are graphs showing representative Differential Scanning Fluorimetry (DSF) melting curves of DF-hIL-12-Fc si in various pharmaceutical formulations after a 1 week incubation at 5° C. (FIGS. 36E-36F) and at 50° C. (FIGS. 36G-36H).

FIGS. 37A-37B are graphs showing the average T_{agg} of DF-hIL-12-Fc si in various pharmaceutical formulations after a 1 week incubation at at 5° C. (FIG. 37A) and at 50° C. (FIG. 37B).

FIGS. 37C-37F are graphs showing representative DSF aggregation curves of DF-hIL-12-Fc si in various pharmaceutical formulations after a 1 week incubation at at 5° C. (FIGS. 37C-37D) and at 50° C. (FIGS. 37E-37F).

FIGS. 38A-38B are graphs showing UV-Vis calculated concentrations of DF-hIL-12-Fc si in various pharmaceutical formulations containing different buffers after a 1 week incubation at 5° C. (FIG. 38A) and at 50° C. (FIG. 38B).

FIGS. 39A-39B are graphs showing pH of various pharmaceutical formulations containing DF-hIL-12-Fc si after a 1 week incubation at 5° C. (FIG. 39A) and at 50° C. (FIG. 39B).

FIGS. 40A-40B are graphs showing Z-average hydrodynamic diameter (FIG. 40A) and polydispersity index (FIG. 40B) of DF-hIL-12-Fc si in various pharmaceutical formulations containing different buffers after a 1 week incubation at 5° C. FIGS. 40C-40D are graphs showing average monomer size and average monomer % Pd of DF-hIL-12-Fc si in various pharmaceutical formulations containing different buffers after a 1 week incubation at 5° C.

FIGS. 40E-40F are graphs showing Z-average hydrodynamic diameter (FIG. 40E) and polydispersity index (FIG. 40F) of DF-hIL-12-Fc si in various pharmaceutical formulations containing different buffers after a 1 week incubation at 50° C. FIGS. 40G-40H are graphs showing average monomer size (FIG. 40G) and average monomer % Pd (FIG. 40H) of DF-hIL-12-Fc si in various pharmaceutical formulations containing different buffers after a 1 week incubation at 50° C.

FIGS. 401-40J show representative Dynamic Light Scattering (DLS) traces used to calculate the data described in FIGS. 40A-40D. FIGS. 40K-40L shows representative DLS traces used to calculate the data described in FIGS. 40E-40H.

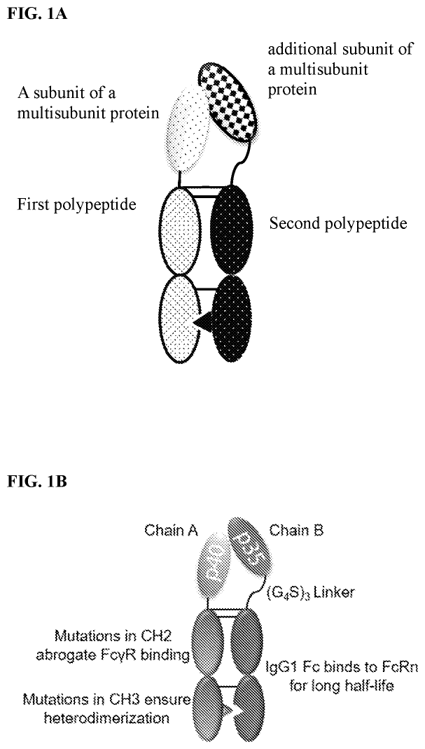

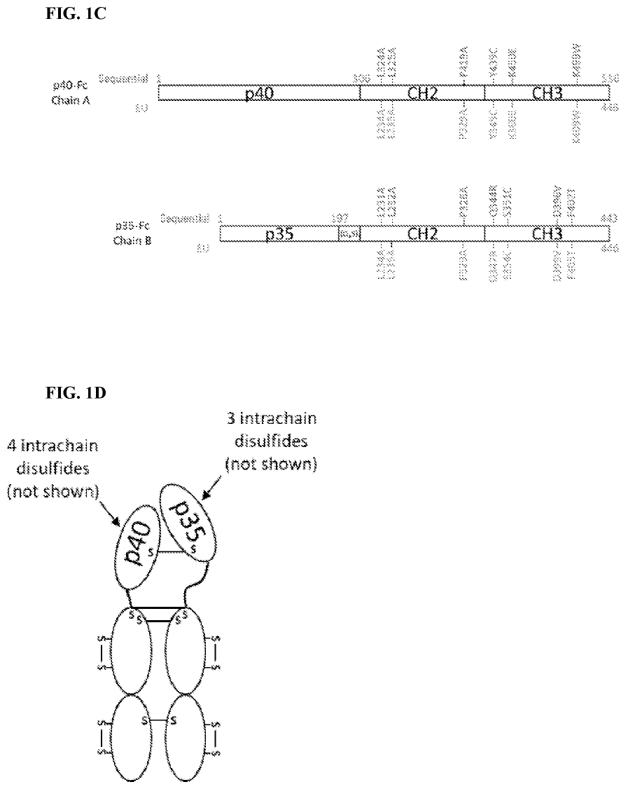

FIG. 41 is a graph showing % purity of DF-hIL-12-Fc si in various pharmaceutical formulations containing different buffers after a 1 week incubation at 50° C., as measured by Size Exclusion Chromatography High-performance Liquid Chromatography (SEC-HPLC).

FIG. 42 is a graph showing % purity of DF-hIL-12-Fc si in various pharmaceutical formulations containing different buffers after a 1 week incubation at 50° C., as measured by Capillary Electrophoresis sodium dodecyl sulfate (CE-SDS).

FIG. 43A is a graph showing UV-Vis calculated concentrations of DF hIL-12-Fc-si at 1 mg/mL in various pharmaceutical formulations containing different buffers and excipients. Referring to FIG. 43A: 1 represents buffer exchange; 2 represents 2-8° C.; 3 represents 50° C.; and 4 reprsents freeze/thaw. FIG. 43B is a graph showing UV-Vis calculated concentrations of DF hIL-12-Fc-si at 10 mg/mL in various pharmaceutical formulations containing different buffers and excipients. Referring to FIG. 43B: 1 represents buffer exchange; 2 represents 2-8° C.; 3. Represents 50° C.; and 4 reprsents freeze/thaw.

FIGS. 44A-44D are graphs showing the average Tₘ₁ (FIGS. 44A and 44B) and Tₘ₂ (FIGS. 44C and 44D) of DF hIL-12-Fc-si at 1 mg/mL and 10 mg/mL in various pharmaceutical formulations.

FIGS. 45A-45D show representative DSF melting curves of DF hIL-12-Fc-si in various pharmaceutical formulations.

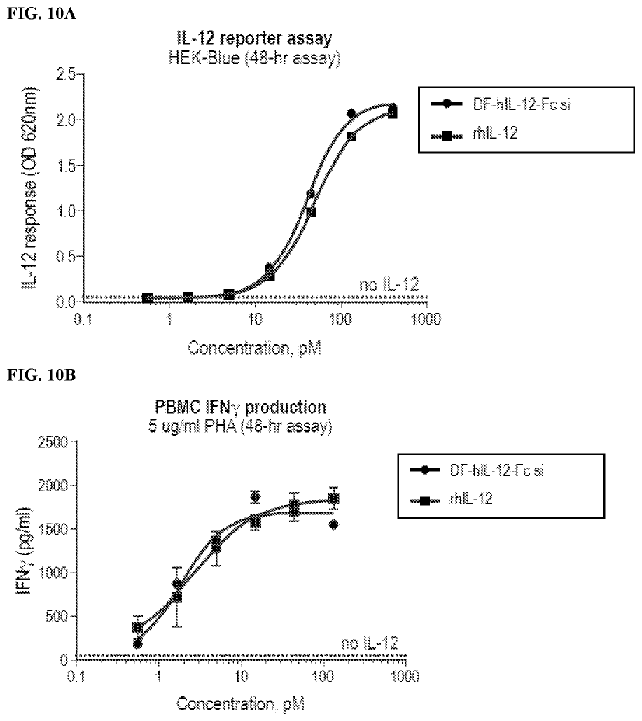

FIGS. 46A-46B are graphs showing the average T_{agg} of DF hIL-12-Fc-si at 1 mg/mL (FIG. 46A) and 10 mg/mL (FIG. 46B) in various pharmaceutical formulations.

FIGS. 46C-46F show representative DSF aggregation curves of DF hIL-12-Fc-si at 1 mg/mL and 10 mg/mL in various pharmaceutical formulations.

FIG. 47A-47F show representative DLS traces showing size distribution of DF hIL-12-Fc-si at 1 mg/mL and 10 mg/mL in various pharmaceutical formulations that were subjected to different stress conditions.

FIGS. 48A-48H show Z-average size (FIGS. 48A-48B), polydispersity index (PDI) (FIGS. 48C-48D), monomer size (48E-48F), and monomer % Pd (FIGS. 48G-48H) of DF hIL-12-Fc-si at 1 mg/mL and 10 mg/mL in various pharmaceutical formulations that were subjected to different stress conditions. In FIGS. 48A-48H: 1 represents 2-8° C., 2 represents 50° C., and 3 represents freeze/thaw.

FIGS. 49A-49D are graphs showing purity of DF hIL-12-Fc-si at 1 mg/mL and 10 mg/mL in various pharmaceutical formulations that were subject to different stress conditions. FIGS. 49A-49B show % main peak after incubation at 50° C. FIGS. 49C-49D show % main peak after incubation at 2-8° C. or freeze-thaw cycle. Referring to FIGS. 49C-49D: 1 represents 2-8° C., and 2 represents freeze/thaw.

FIGS. 50A-50H are graphs showing particle counts by particle sizes in various pharmaceutical formulations containing DF hIL-12-Fc-si that were subject to different stress conditions, as measured by high accuracy liquid particle (HIAC) analysis. FIG. 50A-50B show counts for ≥2 μm particles, FIGS. 50C-50D show counts for ≥5 m particles, FIGS. 50E-50F show counts for ≥10 m particles, and FIGS. 50G-50H show counts for ≥25 m particles. Referring to FIGS. 50A-50H: 1 represents 2-8° C., 2 represents 50° C., and 3 represents freeze/thaw.

FIGS. 51A and 51B are schematic diagrams showing study designs for phase 1 and phase 2 where DF hIL-12-Fc-si will be used as a monotherapy (FIG. 51A) and in a combination therapy with Pembrolizumab (FIG. 51B).

FIGS. 52A and 52B are schematic diagrams showing study designs for phase 1 and phase 2 where DF hIL-12-Fc-si will be used as a monotherapy (FIG. 52A) and in a combination therapy with Nivolumab (FIG. 52B).

### DETAILED DESCRIPTION

The invention provides improvements on heterodimeric Fc-fused proteins, pharmaceutical formulations comprising such proteins, and therapeutic methods using such proteins and pharmaceutical formulations, including for the treatment of cancer.

To facilitate an understanding of the present invention, a number of terms and phrases are defined below.

The terms “a” and “an” as used herein mean “one or more” and include the plural unless the context is inappropriate.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present invention) sufficient to effect beneficial or desired results (e.g., a desired prophylactic or therapeutic effect). An effective amount can be administered in one or more administration(s), application(s) or dosage(s) and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical formulation” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.
(a) Proteins

The present invention provides Fc-fused protein constructs comprising the amino acid sequences of a multisubunit protein. These fusion protein constructs can exhibit a higher serum half-life compared to a native/natural multisubunit protein, improved yield during production, enhanced stability during storage, and/or improved efficacy when used as a therapeutic.
(i) IgG1 Fc-fused proteins

In one aspect, the present invention provides a heterodimeric IgG1 Fc-fused protein comprising: a first polypeptide comprising a first antibody IgG1 Fc domain polypeptide and a second polypeptide comprising a second antibody IgG1 Fc domain polypeptide bound to the first antibody Fc domain, in which the first polypeptide further comprises a first subunit of a multisubunit protein fused by a linker comprising the amino acid sequence of SEQ ID NO:237 or SEQ ID NO:6 to the first antibody Fc domain polypeptide; a second, different subunit of the multisubunit protein is fused to the second antibody Fc domain polypeptide and the subunits of the multisubunit protein are bound to each other; when X₁ of SEQ ID NO: 237 or SEQ ID NO: 6 represents L and/or X₂ of SEQ ID NO: 237 or SEQ ID NO: 6 represents L, at least one of the first antibody Fc domain polypeptide and the second antibody Fc domain polypeptide comprises a Q347R mutation for promoting heterodimerization.

In some embodiments, within the heterodimeric Fc-fused protein, the linker connecting the a first subunit of a multisubunit protein to the first antibody Fc domain polypeptide consists of the amino acid sequence of SEQ ID NO:237 or SEQ ID NO:6.

In certain embodiments, the linker connecting the first subunit of a multisubunit protein to the first antibody Fc domain polypeptide further comprises a spacer peptide. In certain embodiments, the linker comprises a sequence of SEQ ID NO:237 or SEQ ID NO:6, and a spacer peptide.

In certain embodiments, the second, different subunit of the multisubunit protein is fused to the second antibody Fc domain polypeptide by a linker that comprises a sequence of SEQ ID NO:237 or SEQ ID NO:6, and a spacer peptide. In certain embodiments, the second, different subunit of the multisubunit protein is fused to the second antibody Fc domain polypeptide by a linker that consists of the amino acid sequence of SEQ ID NO:237 or SEQ ID NO:6. In certain embodiments, the amino acid sequence of the linker connecting the second, different subunit of the multisubunit protein to the second antibody Fc domain polypeptide is identical to the amino acid sequence of the linker connecting the subunit of the multisubunit protein to the first antibody Fc domain polypeptide.

Any spacer peptide described under the heading “Spacer peptides” can be employed. For example, in certain embodiments, the spacer peptide comprises the amino acid sequence set forth in any one of SEQ ID NOs:107-120. In certain embodiments, the spacer peptide consists of the amino acid sequence set forth in any one of SEQ ID NOs:107-120. In certain embodiments, the linker connecting the subunit of a multisubunit protein to the first antibody Fc domain polypeptide consists of, or consists essentially of, a spacer peptide disclosed herein and a peptide having the sequence of SEQ ID NO:237 or SEQ ID NO:6. In certain embodiments, the linker connecting the second, different subunit of the multisubunit protein to the second antibody Fc domain polypeptide consists of, or consists essentially of, a spacer peptide disclosed herein and a peptide having the sequence of SEQ ID NO:237 or SEQ ID NO:6. In certain embodiments, the spacer peptide is N-terminal to the either or both of the linkers.

In some embodiments, within the heterodimeric Fc-fused protein, the linker connecting the first subunit of the multisubunit protein to the first antibody Fc domain polypeptide comprises the amino acid sequence of SEQ ID NO:239 or SEQ ID NO:9. In some embodiments, within the heterodimeric Fc-fused protein, the linker connecting the first subunit of the multisubunit protein to the first antibody Fc domain polypeptide consists of the amino acid sequence of SEQ ID NO:239 or SEQ ID NO:9.

In some embodiments, within the heterodimeric Fc-fused protein, the linker connecting the first subunit of the multisubunit protein to the first antibody Fc domain polypeptide comprises the amino acid sequence of SEQ ID NO:239). In some embodiments, within the heterodimeric Fc-fused protein, the linker connecting the first subunit of the multisubunit protein to the first antibody Fc domain polypeptide consists of the amino acid sequence SEQ ID NO:239.

In some embodiments, within the heterodimeric Fc-fused protein, the second, different subunit of the multisubunit protein is fused to the second antibody Fc domain polypeptide by a linker comprising the amino acid sequence of SEQ ID NO:10 or SEQ ID NO:244. In some embodiments, the second, different subunit of the multisubunit protein is fused to the second antibody Fe domain polypeptide by a linker consisting of the amino acid sequence of SEQ ID NO:10 or SEQ ID NO:244.

In some embodiments, within the heterodimeric Fc-fused protein, the second, different subunit of the multisubunit protein is fused to the second antibody Fc domain polypeptide by a linker comprising the amino acid sequence of SEQ ID NO:10. In some embodiments, the second, different subunit of the multisubunit protein is fused to the second antibody Fc domain polypeptide by a linker consisting of the amino acid sequence of SEQ ID NO: 10.

In some embodiments, within the heterodimeric Fc-fused protein, the linker connecting the subunit of the multisubunit protein to the first antibody Fc domain polypeptide comprises the amino acid sequence of SEQ ID NO:238 or SEQ ID NO:7. In some embodiments, within the heterodimeric Fc-fused protein, the linker fusing the first subunit of the multisubunit protein to the first antibody Fc domain polypeptide consists of the amino acid sequence of SEQ ID NO:238 or SEQ ID NO:7.

In some embodiments, within the heterodimeric Fc-fused protein, the second, different subunit of the multisubunit protein is fused to the second antibody Fc domain polypeptide by a linker comprising the amino acid sequence of SEQ ID NO:8 or SEQ ID NO:241. In some embodiments, the second, different subunit of the multisubunit protein is fused to the second antibody Fc domain polypeptide by a linker consisting of the amino acid sequence of SEQ ID NO:8 or SEQ ID NO:241.

In some embodiments, within the heterodimeric Fc-fused protein, the second, different subunit of the multisubunit protein is fused to the second antibody Fc domain polypeptide by a linker comprising the amino acid sequence of SEQ ID NO:15 or SEQ ID NO:242. In some embodiments, the second, different subunit of the multisubunit protein is fused to the second antibody Fc domain polypeptide by a linker consisting of the amino acid sequence of SEQ ID NO:15 or SEQ ID NO:242.

In some embodiments, within the heterodimeric Fc-fused protein, the second, different subunit of the multisubunit protein is fused to the second antibody Fc domain polypeptide by a linker comprising the amino acid sequence of SEQ ID NO:16 or SEQ ID NO:243. In some embodiments, the second, different subunit of the multisubunit protein is fused to the second antibody Fc domain polypeptide by a linker consisting of the amino acid sequence of SEQ ID NO:16 or SEQ ID NO:243.

In some embodiments, within the heterodimeric Fc-fused protein, the second, different subunit of the multisubunit protein is fused to the second antibody Fc domain polypeptide by a linker comprising the amino acid sequence of SEQ ID NO:65 or SEQ ID NO:245. In some embodiments, the second, different subunit of the multisubunit protein is fused to the second antibody Fc domain polypeptide by a linker consisting of the amino acid sequence of SEQ ID NO:65 or SEQ ID NO:245.

In some embodiments, within the heterodimeric Fc-fused protein, the second, different subunit of the multisubunit protein is fused to the second antibody Fc domain polypeptide by a linker comprising the amino acid sequence of SEQ ID NO:66 or SEQ ID NO:246. In some embodiments, the second, different subunit of the multisubunit protein is fused to the second antibody Fc domain polypeptide by a linker consisting of the amino acid sequence of SEQ ID NO:66 or SEQ ID NO:246.

In some embodiments, within the heterodimeric Fc-fused protein, the linker connecting the first subunit of the multisubunit protein to the first antibody Fc domain polypeptide comprises the amino acid sequence of SEQ ID NO:11 or SEQ ID NO:240. In some embodiments, within the heterodimeric Fc-fused protein, the linker fusing the first subunit of the multisubunit protein to the first antibody Fc domain polypeptide consists of the amino acid sequence of SEQ ID NO:11 or SEQ ID NO:240.

In some embodiments, within the heterodimeric Fc-fused protein, the second, different subunit of the multisubunit protein is fused to the second antibody Fc domain polypeptide by a linker comprising the amino acid sequence of SEQ ID NO:12 or SEQ ID NO:247. In some embodiments, the second, different subunit of the multisubunit protein is fused to the second antibody Fc domain polypeptide by a linker consisting of the amino acid sequence of SEQ ID NO:12 or SEQ ID NO:247.

In some embodiments, within the heterodimeric Fc-fused protein, the second, different subunit of the multisubunit protein is fused to the second antibody Fc domain polypeptide by a linker comprising the amino acid sequence of SEQ ID NO:67 or SEQ ID NO:248. In some embodiments, the second, different subunit of the multisubunit protein is fused to the second antibody Fc domain polypeptide by a linker consisting of the amino acid sequence of SEQ ID NO:67 or SEQ ID NO:248.

In some embodiments, within the heterodimeric Fc-fused protein, the second, different subunit of the multisubunit protein is fused to the second antibody Fc domain polypeptide by a linker comprising the amino acid sequence of SEQ ID NO:68 or SEQ ID NO:249. In some embodiments, the second, different subunit of the multisubunit protein is fused to the second antibody Fe domain polypeptide by a linker consisting of the amino acid sequence of SEQ ID NO:68 or SEQ ID NO:249.

In certain embodiments the Fc domain polypeptide is that of an IgG1 Fc. In some embodiments, a protein of the current invention includes, a first antibody Fc domain polypeptide and a second antibody Fc domain polypeptide, which are both mutated IgG1 Fc domain polypeptides that promote heterodimerization with each other. For example, if the Fc domain is derived from the Fc of a human IgG1, the Fc domain can comprise an amino acid sequence at least 90% identical to amino acids 234-332 of a human IgG1 antibody, and differ at one or more position(s) selected from the group consisting of Q347, Y349, L351, S354, E356, E357, K360, Q362, S364, T366, L368, K370, N390, K392, T394, D399, S400, D401, F405, Y407, K409, T411, and K439.

In some embodiments, the antibody constant domain can comprise an amino acid sequence at least 90% identical to amino acids 234-332 of a human IgG1 antibody, and differ by one or more substitution(s) selected from the group consisting of Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y407I, Y407V, K409F, K409W, K409D, T411D, T411E, K439D, and K439E. All the amino acid positions in an Fc domain or hinge region disclosed herein are numbered according to EU numbering.

In some embodiments, the first antibody IgG1 Fc domain polypeptide includes one or more mutation(s) selected from K360E and K409W, and the second antibody IgG1 Fc domain polypeptide includes one or more mutation(s) selected from Q347R, D399V, and F405T. In some embodiments, the first antibody IgG1 Fc domain polypeptide includes one or more mutation(s) selected from Q347R, D399V, and F405T, and the second antibody IgG1 Fc domain polypeptide includes one or more mutation(s) selected from K360E and K409W. In some embodiments, the first antibody IgG1 Fc domain polypeptide includes mutations K360E and K409W, and the second antibody IgG1 Fc domain polypeptide includes mutations Q347R, D399V, and F405T. In some embodiments, the first antibody IgG1 Fc domain polypeptide includes mutations Q347R, D399V, and F405T, and the second antibody IgG1 Fc domain polypeptide includes mutations K360E and K409W.

In some embodiments, a heterodimeric Fc-fused protein of the present invention with an IgG1 Fc includes one or more mutation(s) to reduce binding to an FcγR (e.g., FcγRI, FcγRIIA, FcγRIIB, FcγRIIIA, or FcγRIIIB) or a complement component (e.g., C1q) in the first and/or second polypeptides. Such mutations are useful for reducing effector functions. For example, a protein of the present disclosure includes L234A and L235A mutations; L234A, L235A, and P329A mutations; L234A, L235A, and P329G mutations; or L234A, L235E, G237A, A330S, and P331S mutations.

In some embodiments, a heterodimeric Fc-fused protein according to the invention includes a first antibody IgG4 or IgG1 Fc domain polypeptide and the second antibody IgG4 or IgG1 Fc domain polypeptide each containing the mutation P329G or P329A. In specific embodiments, a heterodimeric Fc-fused protein according to the invention comprises a first antibody IgG4 or IgG1 Fc domain polypeptide and a second antibody IgG4 or IgG1 Fc domain polypeptide each comprising the mutation P329A.

In some embodiments, the first IgG1 antibody Fe domain polypeptide and the second, different IgG1 antibody Fe domain polypeptide each contain a mutation selected from A330S and P331S. In some embodiments, the first IgG1 antibody Fe domain polypeptide and the second, different IgG1 antibody Fe domain polypeptide each contain the mutations A330S and P331S.

In certain embodiments, an additional disulfide bond between IgG1 Fc monomers is introduced, which improves the stability of the heterodimer. In an exemplary embodiment, the first antibody Fe domain polypeptide fused to the first subunit of a multisubunit protein includes a Y349C substitution in the CH3 domain, which forms a disulfide bond with an S354C substitution on the second antibody Fe domain polypeptide fused to the second, different subunit of a multisubunit protein. Alternatively, the first antibody Fe domain polypeptide fused to the first subunit of a multisubunit protein includes an S354C substitution in the CH3 domain, which forms a disulfide bond with a Y349C substitution on the second antibody Fe domain polypeptide fused to the second, different subunit of a multisubunit protein.

Any of the IgG1 antibody Fe domain polypeptides provided in Table 2 below can be employed in combination with any of the IgG1 hinge sequences (which, in the current invention, is part or the entirety of a linker connecting the protein sequence of the first subunit of the multisubunit protein to the first IgG1 antibody Fe domain polypeptide, or a linker connecting the additional subunit to the second, different IgG1 antibody Fe domain polypeptide) provided in Table 1 below. Exemplary IgG1 hinge-Fc domain polypeptides are provided in Table 3 below. In certain embodiments, the first and second polypeptides of the Fc-fused protein comprise the amino acid sequences of SEQ ID NOs: 212 and 212; 213 and 214; 215 and 216; 217 and 218; 214 and 213; 216 and 215; or 218 and 217, respectively. In certain embodiments, the first and second polypeptides of the Fc-fused protein comprise the amino acid sequences of SEQ ID NOs:228 and 228; 229 and 230; 231 and 232; 233 and 234; 235 and 236; 230 and 229; 232 and 231; 234 and 233; 236 and 235; 228 and 250; 250 and 228; 250 and 250; 229 and 252; 252 and 229; 251 and 230; 230 and 251; 253 and 232; 232 and 253; 231 and 254; 254 and 231; 255 and 234; 234 and 255; 233 and 256; 256 and 233; 257 and 236; 236 and 257; 258 and 235; or 235 and 258, respectively.
(ii) IgG4 Fc-fused proteins

In one aspect, the current invention provides an improvement on a multisubunit protein. In one aspect the present invention provides a heterodimeric IgG4 Fc-fused protein comprising: a first polypeptide comprising a first antibody IgG4 Fc domain polypeptide and a second polypeptide comprising a second, different antibody IgG4 Fc domain polypeptide bound to the first antibody Fc domain polypeptide, in which the first polypeptide further comprises a first subunit of a multisubunit protein fused by a linker comprising the amino acid sequence of SEQ ID NO:1 to the first antibody IgG4 Fc domain polypeptide; a second, different subunit of the multisubunit protein is fused to the second antibody IgG4 Fc domain polypeptide and the subunits of the multisubunit protein are bound to each other; the first antibody Fc domain polypeptide and the second antibody IgG4 Fc domain polypeptide each contain different mutations promoting heterodimerization.

In some embodiments, within the heterodimeric IgG4 Fc-fused protein, the linker connecting the a first subunit of a multisubunit protein to the first antibody Fc domain polypeptide consists of the amino acid sequence of SEQ ID NO: 1.

In certain embodiments, the linker connecting the protein sequence of a first subunit of a multisubunit protein to the first antibody Fc domain polypeptide further comprises a spacer peptide. In certain embodiments, the linker comprises a sequence of SEQ ID NO:1 and a spacer peptide.

In certain embodiments, the second, different subunit of the multisubunit protein is fused to the second antibody Fc domain polypeptide by a linker that comprises a sequence of SEQ ID NO: 1, and a spacer peptide. In certain embodiments, the second, different subunit of the multisubunit protein is fused to the second antibody Fc domain polypeptide by a linker that consists of the amino acid sequence of SEQ ID NO: 1. In certain embodiments, the amino acid sequence of the linker connecting the second, different subunit of the multisubunit protein to the second antibody Fe domain polypeptide is identical to the amino acid sequence of the linker connecting the subunit of the multisubunit protein to the first antibody Fc domain polypeptide.

Any spacer peptide described under the heading “Spacer peptides” can be employed. For example, in certain embodiments, the spacer peptide comprises the amino acid sequence set forth in any one of SEQ ID NOs: 107-120. In certain embodiments, the spacer peptide consists of the amino acid sequence set forth in any one of SEQ ID NOs: 107-120. In certain embodiments, the linker connecting the subunit of the multisubunit protein to the first antibody Fc domain polypeptide consists of, or consists essentially of, a spacer peptide disclosed herein and SEQ ID NO:1. In certain embodiments, the linker consists of, or consists essentially of, a spacer peptide disclosed herein and SEQ ID NO:1. In certain embodiments, the spacer peptide is N-terminal to the first linker and/or the second linker.

In some embodiments, within the heterodimeric Fc-fused protein, the linker connecting the subunit of the multisubunit protein to the first antibody Fc domain polypeptide comprises the amino acid sequence of SEQ ID NO:2. In some embodiments, the linker fusing the first subunit of the multisubunit protein to the first antibody Fc domain polypeptide consists of the amino acid sequence of SEQ ID NO:2.

In some embodiments, the second, different subunit of the multisubunit protein is fused to the second antibody Fc domain polypeptide by a linker comprising the amino acid sequence of SEQ ID NO:3. In some embodiments, the second, different subunit of the multisubunit protein is fused to the second antibody Fc domain polypeptide by a linker consisting of the amino acid sequence of SEQ ID NO:3.

In some embodiments, the second, different subunit of the multisubunit protein is fused to the second antibody Fc domain polypeptide by a linker comprising the amino acid sequence of SEQ ID NO:13. In some embodiments, the second, different subunit of the multisubunit protein is fused to the second antibody Fc domain polypeptide by a linker consisting of the amino acid sequence of SEQ ID NO:13.

In some embodiments, the second, different subunit of the multisubunit protein is fused to the second antibody Fc domain polypeptide by a linker comprising the amino acid sequence of SEQ ID NO:14. In some embodiments, the second, different subunit of the multisubunit protein is fused to the second antibody Fc domain polypeptide by a linker consisting of the amino acid sequence of SEQ ID NO:14.

In some embodiments, within the heterodimeric Fc-fused protein, the linker connecting the first subunit of the multisubunit protein to the first antibody Fc domain polypeptide comprises the amino acid sequence of SEQ ID NO:4. In some embodiments, the linker fusing the first subunit of the multisubunit protein to the first antibody Fc domain polypeptide consists of the amino acid sequence of SEQ ID NO:4.

In some embodiments, the second, different subunit of the multisubunit protein is fused to the second antibody Fc domain polypeptide by a linker comprising the amino acid sequence of SEQ ID NO:5. In some embodiments, the second, different subunit of the multisubunit protein is fused to the second antibody Fc domain polypeptide by a linker consisting of the amino acid sequence of SEQ ID NO:5.

In some embodiments, the second, different subunit of the multisubunit protein is fused to the second antibody Fc domain polypeptide by a linker comprising the amino acid sequence of SEQ ID NO:63. In some embodiments, the second, different subunit of the multisubunit protein is fused to the second antibody Fc domain polypeptide by a linker consisting of the amino acid sequence of SEQ ID NO:63.

In some embodiments, the second, different subunit of the multisubunit protein is fused to the second antibody Fc domain polypeptide by a linker comprising the amino acid sequence of SEQ ID NO:64. In some embodiments, the second, different subunit of the multisubunit protein is fused to the second antibody Fc domain polypeptide by a linker consisting of the amino acid sequence of SEQ ID NO:64.

In certain embodiments the Fc domain polypeptide is that of an IgG4 Fc. IgG4 is an unstable dimer that can undergo a Fab-arm exchange and pair with other IgG4 antibodies in the body. In certain embodiments, a S228P mutation is introduced within the hinge (which, in the current invention, is part or the entirety of a linker connecting the first subunit of the multisubunit protein to the first IgG4 antibody Fc domain polypeptide, or a linker connecting the additional subunit to the second, different IgG4 antibody Fc domain polypeptide), which increases the stability of the hinge region and reduces the chance for Fab-arm exchange. In certain embodiments, an additional disulfide bond between Fc domain polypeptide monomers is introduced, which improves the stability of the heterodimer. In an exemplary embodiment, the first antibody Fc domain polypeptide linked to the first subunit of the multisubunit protein includes a Y349C substitution in the CH3 domain, which forms a disulfide bond with an S354C substitution on the second antibody Fc domain polypeptide linked to the second, different subunit of the multisubunit protein linked to the second antibody Fc domain polypeptide. Alternatively, the first antibody Fc domain polypeptide linked to the first subunit of the multisubunit protein includes an S354C substitution in the CH3 domain, which forms a disulfide bond with a Y349C substitution on the second antibody Fe domain polypeptide linked to the second, different subunit of the multisubunit protein.

In some embodiments, a protein of the current invention includes, a first antibody Fc domain polypeptide and a second antibody Fc domain polypeptide, which are both mutated IgG4 Fc domain polypeptides that promote heterodimerization with each other.

In some embodiments, the first antibody IgG4 Fc domain polypeptide includes one or more mutation(s) selected from K360E, K370E, and R409W, and the second antibody IgG4 Fc domain polypeptide includes one or more mutation(s) selected from E357N, Q347R, D399V, and F405T. In some embodiments, the first antibody IgG4 Fc domain polypeptide includes mutations K370E and R409W, and the second antibody IgG4 Fc domain polypeptide includes mutations E357N, D399V, and F405T. In some embodiments, the first antibody IgG4 Fc domain polypeptide includes mutations E357N, D399V, and F405T, and the second antibody IgG4 Fc domain polypeptide includes mutations K370E and R409W. In some embodiments, the first antibody IgG4 Fc domain polypeptide includes mutations K360E and R409W, and the second antibody IgG4 Fc domain polypeptide includes mutations Q347R, D399V, and F405T. In some embodiments, the first antibody IgG4 Fc domain polypeptide includes mutations Q347R, D399V, and F405T, and the second antibody IgG4 Fc domain polypeptide includes mutations K360E and R409W.

In some embodiments, a heterodimeric Fc-fused protein of the present invention with an IgG4 Fc includes one or more mutation(s) to reduce binding to an FcγR (e.g., FcγRI, FcγRIIA, FcγRIIB, FcγRIIIA, or FcγRIIIB) or a complement component (e.g., C1q) in the first and/or second polypeptide(s). Such mutations are useful for reducing effector functions. For example, a protein of the present disclosure includes S228P and L235E mutations; S228P, L235E, and P329A mutations; or S228P, L235E, and P329G mutations.

Any of the IgG4 antibody Fc domain polypeptides provided in Table 2 can be employed in combination with any of the IgG4 hinge sequences (which, in the current invention, is part or the entirety of a linker connecting the first subunit of the multisubunit protein to the first IgG4 antibody Fc domain polypeptide, or a linker connecting the second, different subunit of the multisubunit protein to the second, different IgG4 antibody Fc domain polypeptide) provided in Table 1. Exemplary IgG4 hinge-Fc domain polypeptides are provided in Table 3. In certain embodiments, the first and second polypeptides of the Fc-fused protein comprise the amino acid sequences of SEQ ID NOs:205 and 205; 206 and 207; 208 and 209; 210 and 211; 207 and 206; 209 and 208; or 211 and 210, respectively. In certain embodiments, the first and second polypeptides of the Fc-fused protein comprise the amino acid sequences of SEQ ID NOs:219 and 219; 220 and 221; 222 and 223; 224 and 225; 226 and 227; 221 and 220; 223 and 222; 225 and 224; or 227 and 226, respectively.
(b) Disulfide Bonds

Some heterodimeric Fc-fused proteins of the present invention include the native heterodimer disulfide bond between the first subunit of a multisubunit protein and the second, different subunit of the multisubunit protein. For example, in an exemplary embodiment, a heterodimeric Fc-fused protein according to the invention includes a native heterodimer disulfide bond between p35 and p40 subunits of IL-12. Such a protein includes the native disulfide bond between C74 of p35 and C177 of p40.

Some heterodimeric Fc-fused proteins of the present invention include an artificial or engineered heterodimer disulfide bond between the first subunit of a multisubunit protein and the second, different subunit of the multisubunit protein. For example, in an exemplary embodiment, a heterodimeric Fc-fused protein according to the invention includes an artificial or engineered heterodimer disulfide bond between p35 and p40 subunits of IL-12. Such a protein includes an artificial or engineered disulfide bond between V185C of p35 and Y292C of p40.

Some heterodimeric Fc-fused proteins of the present invention include the native heterodimer disulfide bond between the first subunit of a multisubunit protein and the second, different subunit of the multisubunit protein, and an artificial or engineered heterodimer disulfide bond between the first subunit of a multisubunit protein and the second, different subunit of the multisubunit protein. For example, in an exemplary embodiment, a native heterodimer disulfide bond between p35 and p40 subunits of IL-12, and includes an artificial or engineered heterodimer disulfide bond between p35 and p40 subunits of IL-12. Such a protein includes the native disulfide bond between C74 of p35 and C177 of p40, and an artificial or engineered disulfide bond between V185C of p35 and Y292C of p40.

Some heterodimeric Fc-fused proteins of the present invention are engineered to remove the native disulfide bond, and to replace it with a non-native artificial or engineered disulfide bond. For example, in an exemplary embodiment, a heterodimeric Fc-fused protein according to the invention includes p35 of IL-12 in which the native C74 is mutated to serine, and a p40 of IL-12 in which the native C177 is mutated to serine, thereby removing the native disulfide bond between p35 and p40 subunits of IL-12. To this mutated IL-12, two new mutations are introduced, V185C on p35 and Y292C on p40, thereby introducing a non-native artificial or engineered disulfide bond.
(c) Sequences of components of Fc-fused polypeptides

Exemplary heterodimeric Fe-fused proteins of the present invention are constructed with any one of the IgG1 or IgG4 Fe variant sequenees and any one of the corresponding linker sequenees described in the Tables 1-2 below. The fusion protein constructs of the present invention can confer a higher serum half-life compared to a native/natural multisubunit protein, improve yield of the proteins during production, enhance stability during storage, and/or improve efficacy when used as a therapeutic.

Any of the IgG4 antibody Fe variant domain polypeptides provided in Table 2 below can be employed in combination with any of the IgG4 hinge sequenees provided in Table 1 below. Similarly, any of the IgG1 antibody Fe variant domain polypeptides provided in Table 2 below can be employed in combination with any of the IgG1 hinge sequenees provided in Table 1 below. Exemplary IgG1 hinge-Fe domain polypeptides are provided in Table 3 below.

TABLE 1
Linker Variants

| Hinge | Amino Acid Sequence |
|---|---|
| IgG4 hinge consensus | SEQ ID NO:1 |
| IgG4 hinge S228P | SEQ ID NO:2 |
| IgG4 hinge S228P/L235E | SEQ ID NO:4 |
| IgG1 hinge consensus | SEQ ID NO:6 |
| IgG1 hinge C220S | SEQ ID NO:7 |
| IgG1 hinge C220S/L234A/L235A | SEQ ID NO:9 |
| IgG1 hinge C220S/L234A/L235E/G237A | SEQ ID NO:11 |
| IgG1 hinge ΔE216 | SEQ ID NO:237 |
| IgG1 hinge ΔE216/C220S | SEQ ID NO:238 |
| IgG1 hinge ΔE216/C220S/L234A/L235A | SEQ ID NO:239 |
| IgG1 hinge ΔE216/C220S/L234A/L235E/G237A | SEQ ID NO:240 |

TABLE 2
IgG4 Fc and IgG1 Fc Wild-type Sequences; and Exemplary IgG4 Antibody Fc Variant
and IgG1 Antibody Fc Variant Sequences (amino acid substitutions are indicated in bold and
underline)

| Fc domain | Amino Acid Sequence* |
|---|---|
| IgG4 Fc wild-type | SEQ ID NO:205 |
| IgG4 Fc Y349C/K370E/R409W | SEQ ID NO:206 |
| IgG4 Fc S354C/E357N/D399V/F405T | SEQ ID NO:207 |
| IgG4 Fc Y349C/K360E/R409W | SEQ ID NO:208 |
| IgG4 Fc Q347R/S354C/D399V/F405T | SEQ ID NO:209 |
| IgG4 Fc P329A/Y349C/K360E/R409W | SEQ ID NO:210 |
| IgG4 Fc P329A/S354C/Q347R/D399V/ | SEQ ID NO:211 |
| F405T | |
| IgG1 Fc wild-type | SEQ ID NO:212 |
| IgG1 Fc Y349C/K360E/K409W | SEQ ID NO:213 |
| IgG1 Fc Q347R/S354C/D399V/F405T | SEQ ID NO:214 |
| IgG1 Fc P329A/Y349C/K360E/K409W | PSVFLFPPKPKDTLMISRTPEVTCVVVDVS |
| | HEDPEVKFNWYVDGVEVHNAKTKPREEQ |
| | YNSTYRVVSVLTVLHQDWLNGKEYKCK |
| | VSNKALAAPIEKTISKAKGQPREPQVCTLP |
| | PSRDELTENQVSLTCLVKGFYPSDIAVEW |
| | ESNGQPENNYKTTPPVLDSDGSFFLYSWL |
| | TVDKSRWQQGNVFSCSVMHEALHNHYT |
| | QKSLSLSPG (SEQ ID NO:215) |
| IgG1 Fc | PSVFLFPPKPKDTLMISRTPEVTCVVVDVS |
| P329A/Q347R/S354C/D399V/F405T | HEDPEVKFNWYVDGVEVHNAKTKPREEQ |
| | YNSTYRVVSVLTVLHQDWLNGKEYKCK |
| | VSNKALAAPIEKTISKAKGQPREPRVYTLP |
| | PCRDELTKNQVSLTCLVKGFYPSDIAVEW |
| | ESNGQPENNYKTTPPVLVSDGSFTLYSKL |
| | TVDKSRWQQGNVFSCSVMHEALHNHYT |
| | QKSLSLSPG (SEQ ID NO:216) |
| IgG1 Fc A330S/P331S/Y349C/K360E/ | SEQ ID NO:217 |
| K409W | |
| IgG1 Fc A330S/P331S/Q347R/S354C/ | SEQ ID NO:218 |
| D399V/F405T | |

*The amino acid sequences can further comprise a lysine (K) at the C-terminus.

TABLE 3
S228P mutated IgG4 Hinge-Fc (wild-type); Exemplary S228P mutated IgG4 Hinge-Fc
Variants or Hinge Portion-Fc Variants; C220S mutated IgG1 Hinge-Fc (wild-type); Exemplary
C220S mutated IgG1 Hinge-Fc Variants or Hinge Portion-Fc Variants

| | Amino Acid |
|---|---|
| Linker-Fc | Sequence* |
| IgG4 hinge-Fc S228P | SEQ ID NO:219 |
| IgG4 hinge-Fc S228P/Y349C/K370E/R409W | SEQ ID NO:220 |
| IgG4 hinge-Fc S228P/S354C/E357N/D399V/F405T | SEQ ID NO:221 |
| IgG4 hinge-Fc S228P/Y349C/K360E/R409W | SEQ ID NO:222 |
| IgG4 hinge-Fc S228P/Q347R/S354C/D399V/ F405T | SEQ ID NO:223 |
| IgG4 hinge-Fc S228P/L235E/Y349C/K360E/R409W | SEQ ID NO:224 |
| IgG4 hinge-Fc S228P/L235E/S354C/Q347R/ D399V/F405T | SEQ ID NO:225 |
| IgG4 hinge-Fc S228P/L235E/P329A/Y349C/K360E/R409W | SEQ ID NO:226 |
| IgG4 hinge-Fc S228P/L235E/ P329A/S354C/Q347R/D399V/F405T | SEQ ID NO:227 |
| IgG1 hinge-Fc C220S | SEQ ID NO:228 |
| IgG1 hinge-Fc ΔE216/C220S | SEQ ID NO:250 |
| IgG1 hinge-Fc C220S/Y349C/K360E/K409W | SEQ ID NO:229 |
| IgG1 hinge-Fc ΔE216/C220S/Y349C/K360E/ K409W | SEQ ID NO:251 |
| IgG1 hinge-Fc C220S/Q347R/S354C/D399V/ F405T | SEQ ID NO:230 |
| IgG1 hinge-Fc ΔE216/C220S/Q347R/S354C/ D399V/F405T | SEQ ID NO:252 |
| IgG1 hinge-Fc C220S/L234A/L235A/Y349C/K360E/K409W | SEQ ID NO:231 |
| IgG1 hinge-Fc ΔE216/C220S/L234A/L235A/Y349C/K360E/K409W | SEQ ID NO:253 |
| IgG1 hinge-Fc C220S/L234A/L235A/Q347R/S354C/D399V/F405T | SEQ ID NO:232 |
| IgG1 hinge-Fc ΔE216/C220S/L234A/L235A/ | SEQ ID NO:254 |
| Q347R/S354C/D399V/F405T | |
| IgG1 hinge-Fc C220S/L234A/L235A/P329A/Y349C/K360E/K409W | SEQ ID NO:233 |
| IgG1 hinge-Fc ΔE216/C220S/L234A/L235A/ | SEQ ID NO:255 |
| P329A/Y349C/K360E/K409W | |
| IgG1 hinge-Fc C220S/L234A/L235A/P329A/ | SEQ ID NO:234 |
| Q347R/S354C/D399V/F405T | |
| IgG1 hinge-Fc ΔE216/C220S/L234A/L235A/ | SEQ ID NO:256 |
| P329A/Q347R/S354C/D399V/F405T | |
| IgG1 hinge-Fc C220S/L234A/L235E/G237A/ | SEQ ID NO:235 |
| A330S/P331S/Y349C/K360E/K409W | |
| IgG1 hinge-Fc ΔE216/C220S/L234A/L235E/ | SEQ ID NO:257 |
| G237A/A330S/P331S/Y349C/K360E/K409W | |
| IgG1 hinge-Fc C220S/L234A/L235E/G237A/ | SEQ ID NO:236 |
| A330S/P331S/Q347R/S354C/D399V/F405T | |
| IgG1 hinge-Fc ΔE216/C220S/L234A/L235E/ | SEQ ID NO:258 |
| G237A/A330S/P331S/Q347R/S354C/D399V/F405T | |

*The amino acid sequences can further comprise a lysine (K) at the C-terminus.

| | | |
|---|---|---|
| | Sequences of | |
| | Heterodimeric | Type of IgG (IL-12 subunit); |
| Linker | Fc-fused | mutations/ |
| Sequence | Proteins* | substitutions |

Construct 101

| | | |
|---|---|---|
| SEQ ID NO:2 | SEQ ID NO:129 | IgG4 (IL-12 p40); |
| | | Heterodimerization mutations: K370E/R409W; |
| | | Substitution for stabilizing disulfide bond: Y349C; |
| | | Substitution for preventing Fab-arm exchange and improving |
| | | thermostability: S228P |
| SEQ ID NO:3 | SEQ ID NO:130 | IgG4 (IL-12 p35); |
| | | Heterodimerization mutations: E357N/D399V/F405T; |
| | | Substitution for stabilizing disulfide bond: S354C; |
| | | Substitution for preventing Fab-arm exchange and improving |
| | | thermostability: S228P |

Construct 102

| | | |
|---|---|---|
| SEQ ID NO:2 | SEQ ID NO:131 | IgG4 (IL-12 p40); |
| | | Heterodimerization mutations: K360E/R409W; |
| | | Substitution for stabilizing disulfide bond: Y349C; |
| | | Substitution for preventing Fab-arm exchange and improving |
| | | thermostability: S228P |
| SEQ ID NO:3 | SEQ ID NO:132 | IgG4 (IL-12 p35); |
| | | Heterodimerization mutations: Q347R/D399V/F405T; |
| | | Substitution for stabilizing disulfide bond: S354C; |
| | | Substitution for preventing Fab-arm exchange and improving |
| | | thermostability: S228P |

Construct 103

| | | |
|---|---|---|
| SEQ ID NO:4 | SEQ ID NO:133 | IgG4 (IL-12 p40); |
| | | Heterodimerization mutations: K360E/R409W; |
| | | Substitution for stabilizing disulfide bond: Y349C; |
| | | Substitution for preventing Fab-arm exchange and improving |
| | | thermostability: S228P; |
| | | Substitutions for reducing FcγR binding: S228P and L235E |
| SEQ ID NO:5 | SEQ ID NO:134 | IgG4 (IL-12 p35); |
| | | Heterodimerization mutations: Q347R/D399V/F405T; |
| | | Substitution for stabilizing disulfide bond: S354C; |
| | | Substitution for preventing Fab-arm exchange and improving |
| | | thermostability: S228P; |
| | | Substitutions for reducing FcγR binding: S228P and L235E |

Construct 104

| | | |
|---|---|---|
| SEQ ID NO:4 | SEQ ID NO:135 | IgG4 (IL-12 p40); |
| | | Heterodimerization mutations: K360E/R409W; |
| | | Substitution for stabilizing disulfide bond: Y349C; |
| | | Substitution for preventing Fab-arm exchange and improving |
| | | thermostability: S228P; |
| | | Substitutions for reducing FcγR binding: S228P, L235E, |
| | | P329A |
| SEQ ID NO:5 | SEQ ID NO:136 | IgG4 (IL-12 p35); |
| | | Heterodimerization mutations: Q347R/D399V/F405T; |
| | | Substitution for stabilizing disulfide bond: S354C; |
| | | Substitution for preventing Fab-arm exchange and improving |
| | | thermostability: S228P; |
| | | Substitutions for reducing FcγR binding: S228P, L235E, |
| | | P329A |

Construct 106

| | | |
|---|---|---|
| SEQ ID NO:7 | SEQ ID NO:137 | IgG1 (IL-12 p40); |
| OR | OR | Heterodimerization mutations: K360E/K409W; |
| SEQ ID | SEQ ID NO:259 | Substitution for stabilizing disulfide bond: Y349C; |
| NO:238 | | C220 in the upper hinge is mutated to S |
| SEQ ID NO:8 | SEQ ID NO:138 | IgG1 (IL-12 p35); |
| | | Heterodimerization mutations: Q347R/D399V/F405T; |
| | | Substitution for stabilizing disulfide bond: S354C; |
| | | C220 in the upper hinge is mutated to S |

Construct 107

| | | |
|---|---|---|
| SEQ ID NO:9 | SEQ ID NO:139 | IgG1FcSilent; |
| OR | OR | (IL-12 p40); |
| SEQ ID | SEQ ID NO:260 | Heterodimerization mutations: K360E/K409W; |
| NO:239 | | Substitution for stabilizing disulfide bond: Y349C; |
| | | Substitutions for reducing FcγR binding: L234A, L235A; |
| | | C220 in the upper hinge is mutated to S |
| SEQ ID | SEQ ID NO:140 | IgG1FcSilent; |
| NO:10 | | (IL-12 p35); |
| | | Heterodimerization mutations: Q347R/D399V/F405T; |
| | | Substitution for stabilizing disulfide bond: S354C; |
| | | Substitutions for reducing FcγR binding: L234A, L235A; |
| | | C220 in the upper hinge is mutated to S |

Construct 108

| | | |
|---|---|---|
| SEQ ID NO:9 | SEQ ID NO:141 | IgG1FcSilent; |
| OR | OR | (IL-12 p40); |
| SEQ ID | SEQ ID NO:261 | Heterodimerization mutations: K360E/K409W; |
| NO:239 | | Substitution for stabilizing disulfide bond: Y349C; |
| | | Substitutions for reducing FcγR binding: L234A, L235A, |
| | | P329A; |
| | | C220 in the upper hinge is mutated to S |
| SEQ ID | SEQ ID NO:142 | IgG1FcSilent; |
| NO:10 | | (IL-12 p35); |
| | | Heterodimerization mutations: Q347R/D399V/F405T; |
| | | Substitution for stabilizing disulfide bond: S354C; |
| | | Substitutions for reducing FcγR binding: L234A, L235A, |
| | | P329A; |
| | | C220 in the upper hinge is mutated to S |

Construct 110

| | | |
|---|---|---|
| SEQ ID | SEQ ID NO:143 | IgG1 Fc; |
| NO:11 | OR | (IL-12 p40); |
| OR | SEQ ID NO:262 | Heterodimerization mutations: K360E/K409W; |
| SEQ ID | | Substitution for stabilizing disulfide bond: Y349C ; |
| NO:240 | | Substitutions for reducing FcγR and C1q binding: L234A, |
| | | L235E, G237A, A330S, P331S |
| SEQ ID | SEQ ID NO:144 | IgG1 Fc; |
| NO:12 | | (IL-12 p35); |
| | | Heterodimerization mutations: Q347R/D399V/F405T; |
| | | Substitution for stabilizing disulfide bond: S354C; |
| | | Substitutions for reducing FcγR and C1q binding: L234A, |
| | | L235E, G237A, A330S, P331S |

Construct 111

| | | |
|---|---|---|
| SEQ ID NO:2 | SEQ ID NO:145 | IgG4 Fc; |
| | | (IL-12 p40); |
| | | Heterodimerization mutations: K370E/R409W; |
| | | Substitution for stabilizing disulfide bond: Y349C; |
| | | Substitution for preventing Fab-arm exchange and improving |
| | | thermostability: S228P |
| SEQ ID NO:2 | SEQ ID NO:146 | IgG4 Fc; |
| | | (IL-12 p35); |
| | | Heterodimerization mutations: E357N/D399V/F405T; |
| | | Substitution for stabilizing disulfide bond: S354C; |
| | | Substitution for preventing Fab-arm exchange and improving |
| | | thermostability: S228P |

Construct 112

| | | |
|---|---|---|
| SEQ ID NO:2 | SEQ ID NO:147 | IgG4 Fc; |
| | | (IL-12 p40); |
| | | Heterodimerization mutations: K370E/R409W; |
| | | Substitution for stabilizing disulfide bond: Y349C; |
| | | Substitution for preventing Fab-arm exchange and improving |
| | | thermostability: S228P; |
| SEQ ID | SEQ ID NO:148 | IgG4 Fc; |
| NO:13 | | (IL-12 p35); |
| | | Heterodimerization mutations: E357N/D399V/F405T; |
| | | Substitution for stabilizing disulfide bond: S354C; |
| | | Substitution for preventing Fab-arm exchange and improving |
| | | thermostability: S228P; |

Construct 113

| | | |
|---|---|---|
| SEQ ID NO:2 | SEQ ID NO:149 | IgG4Fc; |
| | | (IL-12 p40); |
| | | Heterodimerization mutations: K370E/R409W; |
| | | Substitution for stabilizing disulfide bond: Y349C; |
| | | Substitution for preventing Fab-arm exchange and improving |
| | | thermostability: S228P |
| SEQ ID | SEQ ID NO:150 | IgG4 Fc; |
| NO:14 | | (IL-12 p35); |
| | | Heterodimerization mutations: E357N/D399V/F405T; |
| | | Substitution for stabilizing disulfide bond: S354C; |
| | | Substitution for preventing Fab-arm exchange and improving |
| | | thermostability: S228P |

Construct 114

| | | |
|---|---|---|
| SEQ ID NO:7 | SEQ ID NO: 151 | IgG1 Fc; |
| OR | OR | (IL-12 p40); |
| SEQ ID | SEQ ID NO:263 | Heterodimerization mutations: K360E/K409W; |
| NO:238 | | Substitution for stabilizing disulfide bond: Y349C; |
| | | C220 in the upper hinge is mutated to S |
| SEQ ID NO:7 | SEQ ID NO:152 | IgG1 Fc; |
| | | (IL-12 p35); |
| | | Heterodimerization mutations: Q347R/D399V/F405T; |
| | | Substitution for stabilizing disulfide bond: S354C; |
| | | C220 in the upper hinge is mutated to S |

Construct 115

| | | |
|---|---|---|
| SEQ ID NO:7 | SEQ ID NO:153 | IgG1 Fc; |
| OR | OR | (IL-12 p40); |
| SEQ ID | SEQ ID NO:264 | Heterodimerization mutations: K360E/K409W; |
| NO:238 | | Substitution for stabilizing disulfide bond: Y349C; |
| | | C220 in the upper hinge is mutated to S |
| SEQ ID | SEQ ID NO:154 | IgG1 Fc; |
| NO:15 | | (IL-12 p35); |
| | | Heterodimerization mutations: Q347R/D399V/F405T; |
| | | Substitution for stabilizing disulfide bond: S354C; |
| | | C220 in the upper hinge is mutated to S |

Construct 116

| | | |
|---|---|---|
| SEQ ID NO:7 | SEQ ID NO:155 | IgG1 Fc; |
| OR | OR | (IL-12 p40); |
| SEQ ID | SEQ ID NO:265 | Heterodimerization mutations: K360E/K409W; |
| NO:238 | | Substitution for stabilizing disulfide bond: Y349C; |
| | | C220 in the upper hinge is mutated to S |
| SEQ ID | SEQ ID NO:156 | IgG1 Fc; |
| NO:16 | | (IL-12 p35); |
| | | Heterodimerization mutations: Q347R/D399V/F405T; |
| | | Substitution for stabilizing disulfide bond: S354C; |
| | | C220 in the upper hinge is mutated to S |

Construct 117

| | | |
|---|---|---|
| SEQ ID NO:8 | SEQ ID NO:157 | IgG1 Fc; |
| OR | OR | (IL-12 p35); |
| SEQ ID | SEQ ID NO:266 | Heterodimerization mutations: K360E/K409W; |
| NO:241 | | Substitution for stabilizing disulfide bond: Y349C; |
| | | C220 in the upper hinge is mutated to S |
| SEQ ID NO:7 | SEQ ID NO:158 | IgG1 Fc; |
| | | (IL-12 p40); |
| | | Heterodimerization mutations: Q347R/D399V/F405T; |
| | | Substitution for stabilizing disulfide bond: S354C; |
| | | C220 in the upper hinge is mutated to S |

Construct 118

| | | |
|---|---|---|
| SEQ ID | SEQ ID NO:159 | IgG1 (IL-12 p40); |
| NO:15 | OR | Heterodimerization mutations: K360E/K409W; |
| OR | SEQ ID NO:267 | Substitution for stabilizing disulfide bond: Y349C; |
| SEQ ID | | C220 in the upper hinge is mutated to S |
| NO:242 | | |
| SEQ ID | SEQ ID NO:160 | IgG1 (IL-12 p35); |
| NO:15 | | Heterodimerization mutations: Q347R/D399V/F405T; |
| | | Substitution for stabilizing disulfide bond: S354C; |
| | | C220 in the upper hinge is mutated to S |

Construct 119

| | | |
|---|---|---|
| SEQ ID | SEQ ID NO:161 | IgG1 (IL-12 p40); |
| NO:15 | OR | Heterodimerization mutations: K360E/K409W; |
| OR | SEQ ID NO:268 | p40 substitutions: C177S, Y292C; |
| SEQ ID | | (native disulfide bond between subunits is deleted) |
| NO:242 | | |
| SEQ ID | SEQ ID NO:162 | IgG1 (IL-12 p35); |
| NO:15 | | Heterodimerization mutations: Q347R/D399V/F405T; |
| | | p35 substitutions: C74S V185C; |
| | | (native disulfide bond between subunits is deleted) |

Construct 119-1

| | | |
|---|---|---|
| SEQ ID NO:2 | SEQ ID NO:163 | IgG4 (IL-12 p40); |
| | | Heterodimerization mutations: K370E/R409W; |
| | | p40 substitutions: C177S, Y292C; |
| | | (native disulfide bond between subunits is deleted); |
| | | Substitution for preventing Fab-arm exchange and improving |
| | | thermostability: S228P |
| SEQ ID NO:3 | SEQ ID NO:164 | IgG4 (IL-12 p35); |
| | | Heterodimerization mutations: E357N/D399V/F405T; |
| | | p35 substitutions: C74S V185C; |
| | | Substitution for preventing Fab-arm exchange and improving |
| | | thermostability: S228P |

Construct 119-2

| | | |
|---|---|---|
| SEQ ID NO:2 | SEQ ID NO:165 | IgG4 (IL-12 p40); |
| | | Heterodimerization mutations: K360E/R409W; |
| | | p40 substitutions: C177S, Y292C; |
| | | (native disulfide bond between subunits is deleted); |
| | | Substitution for preventing Fab-arm exchange and improving |
| | | thermostability: S228P |
| SEQ ID NO:3 | SEQ ID NO:166 | IgG4 (IL-12 p35); |
| | | Heterodimerization mutations: Q347R/D399V/F405T; |
| | | p35 substitutions: |
| | | C74S V185C; |
| | | Substitution for preventing Fab-arm exchange and improving |
| | | thermostability: S228P |

Construct 119-3

| | | |
|---|---|---|
| SEQ ID NO:4 | SEQ ID NO:167 | IgG4 (IL-12 p40) |
| | | Heterodimerization mutations: K360E/R409W; |
| | | p40 substitutions: C177S, Y292C; |
| | | (native disulfide bond between subunits is deleted); |
| | | Substitution for preventing Fab-arm exchange and improving |
| | | thermostability: S228P; |
| | | Substitutions for reducing FcγR binding: S228P and L235E |
| SEQ ID NO:5 | SEQ ID NO:168 | IgG4 (IL-12 p35); |
| | | Heterodimerization mutations: Q347R/D399V/F405T; |
| | | p35 substitutions: |
| | | C74S V185C; |
| | | Substitution for preventing Fab-arm exchange and improving |
| | | thermostability: S228P; |
| | | Substitutions for reducing FcγR binding: S228P and L235E |

Construct 119-4

| | | |
|---|---|---|
| SEQ ID NO:4 | SEQ ID NO:169 | IgG4 (IL-12 p40); |
| | | Heterodimerization mutations: K360E/R409W; |
| | | p40 substitutions: C177S, Y292C; |
| | | (native disulfide bond between subunits is deleted); |
| | | Substitution for preventing Fab-arm exchange and improving |
| | | thermostability: S228P; |
| | | Substitutions for reducing FcγR binding: S228P, L235E, |
| | | P329A |
| SEQ ID NO:5 | SEQ ID NO:170 | IgG4 (IL-12 p35); |
| | | Heterodimerization mutations: Q347R/D399V/F405T; |
| | | p35 substitutions: C74S V185C; |
| | | Substitution for preventing Fab-arm exchange and improving |
| | | thermostability: S228P; |
| | | Substitutions for reducing FcγR binding: S228P, L235E, |
| | | P329A |

Construct 119-5

| | | |
|---|---|---|
| SEQ ID NO:7 | SEQ ID NO:171 | IgG1 (IL-12 p40); |
| OR | OR | Heterodimerization mutations: K360E/K409W; |
| SEQ ID | SEQ ID NO:269 | p40 substitutions: C177S, Y292C; |
| NO:238 | | (native disulfide bond between subunits is deleted); |
| | | C220 in the upper hinge is mutated to S |
| SEQ ID NO:8 | SEQ ID NO:172 | IgG1 (IL-12 p35); |
| | | Heterodimerization mutations: Q347R/D399V/F405T; |
| | | p35 substitutions: C74S V185C; |
| | | C220 in the upper hinge is mutated to S |

Construct 119-6

| | | |
|---|---|---|
| SEQ ID NO:9 | SEQ ID NO:173 | IgG1 (IL-12 p40); |
| OR | OR | Heterodimerization mutations: K360E/K409W; |
| | SEQ ID NO:270 | p40 substitutions: C177S, Y292C; |
| SEQ ID | | (native disulfide bond between subunits is deleted); |
| NO:239 | | Substitutions for reducing FcγR binding: L234A, L235A; |
| | | C220 in the upper hinge is mutated to S |
| SEQ ID | SEQ ID NO:174 | IgG1 (IL-12 p35); |
| NO:10 | | Heterodimerization mutations: Q347R/D399V/F405T; |
| | | p35 substitutions: C74S V185C; |
| | | Substitutions for reducing FcγR binding: L234A, L235A; |
| | | C220 in the upper hinge is mutated to S |

Construct 119-7

| | | |
|---|---|---|
| SEQ ID NO:9 | SEQ ID NO:175 | IgG1 (IL-12 p40); |
| OR | OR | Heterodimerization mutations: K360E/K409W; |
| SEQ ID | SEQ ID NO:271 | p40 substitutions: C177S, Y292C; |
| NO:239 | | (native disulfide bond between subunits is deleted); |
| | | Substitutions for reducing FcγR binding: L234A, L235A, |
| | | P329A; |
| | | C220 in the upper hinge is mutated to S |
| SEQ ID | SEQ ID NO:176 | IgG1 (IL-12 p35); |
| NO:10 | | Heterodimerization mutations: Q347R/D399V/F405T; |
| | | p35 substitutions: C74S, V185C; |
| | | Substitutions for reducing FcγR binding: L234A, L235A, |
| | | P329A; |
| | | C220 in the upper hinge is mutated to S |

Construct 119-8

| | | |
|---|---|---|
| SEQ ID | SEQ ID NO:177 | IgG1 (IL-12 p40); |
| NO:11 | OR | Heterodimerization mutations: K360E/K409W; |
| OR | SEQ ID NO:272 | p40 substitutions: C177S, Y292C; |
| SEQ ID | | (native disulfide bond between subunits is deleted); |
| NO:240 | | Substitutions for reducing FcγR and C1q binding: L234A, |
| | | L235E, G237A, A330S, P331S; |
| | | C220 in the upper hinge is mutated to S |
| SEQ ID | SEQ ID NO:178 | IgG1 (IL-12 p35); |
| NO:12 | | Heterodimerization mutations: Q347R/D399V/F405T; |
| | | p35 substitutions: C74S, V185C; |
| | | Substitutions for reducing FcγR and C1q binding: L234A, |
| | | L235E, G237A, A330S, P331S; |
| | | C220 in the upper hinge is mutated to S |

Construct 120

| | | |
|---|---|---|
| SEQ ID NO:2 | SEQ ID NO:179 | IgG4 (IL-12 p40); |
| | | Heterodimerization mutations: K370E/R409W; |
| | | p40 substitution: Y292C (native disulfide bond between |
| | | subunits is preserved); |
| | | Substitution for preventing Fab-arm exchange and improving |
| | | thermostability: S228P |
| SEQ ID NO:3 | SEQ ID NO:180 | IgG4 (IL-12 p35); |
| | | Heterodimerization mutations: E357N/D399V/F405T; |
| | | p35 substitution: V185C (native disulfide bond between |
| | | subunits is preserved); |
| | | Substitution for preventing Fab-arm exchange and improving |
| | | thermostability: S228P; |

Construct 120-1

| | | |
|---|---|---|
| SEQ ID NO:2 | SEQ ID NO:181 | IgG4 (IL-12 p40); |
| | | Heterodimerization mutations: K360E/R409W; |
| | | p40 substitution: Y292C (native disulfide bond between |
| | | subunits is preserved); |
| | | Substitution for preventing Fab-arm exchange and improving |
| | | thermostability: S228P |
| SEQ ID NO:3 | SEQ ID NO:182 | IgG4 (IL-12 p35); |
| | | Heterodimerization mutations: Q347R/D399V/F405T; |
| | | p35 substitution: V185C (native disulfide bond between |
| | | subunits is preserved); |
| | | Substitution for preventing Fab-arm exchange and improving |
| | | thermostability: S228P |

Construct 120-2

| | | |
|---|---|---|
| SEQ ID NO:4 | SEQ ID NO:183 | IgG4 (IL-12 p40); |
| | | Heterodimerization mutations: K360E/R409W; |
| | | p40 substitution: Y292C (native disulfide bond between |
| | | subunits is preserved); |
| | | Substitution for preventing Fab-arm exchange and improving |
| | | thermostability: S228P; |
| | | Substitutions for reducing FcγR binding: S228P and L235E |
| SEQ ID NO:5 | SEQ ID NO: 184 | IgG4 (IL-12 p35); |
| | | Heterodimerization mutations: Q347R/D399V/F405T; |
| | | p35 substitution: V185C (native disulfide bond between |
| | | subunits is preserved); |
| | | Substitution for preventing Fab-arm exchange and improving |
| | | thermostability: S228P; |
| | | Substitutions for reducing FcγR binding: S228P and L235E |

Construct 120-3

| | | |
|---|---|---|
| SEQ ID NO:4 | SEQ ID NO:185 | IgG4 (IL-12 p40); |
| | | Heterodimerization mutations: K360E/R409W; |
| | | p40 substitution: Y292C (native disulfide bond between |
| | | subunits is preserved); |
| | | Substitution for preventing Fab-arm exchange and improving |
| | | thermostability: S228P; |
| | | Substitutions for reducing FcγR binding: S228P, L235E, |
| | | P329A |
| SEQ ID NO:5 | SEQ ID NO:186 | IgG4 (IL-12 p35); |
| | | Heterodimerization mutations: Q347R/D399V/F405T; |
| | | p35 substitution: V185C (native disulfide bond between |
| | | subunits is preserved); |
| | | Substitution for preventing Fab-arm exchange and improving |
| | | thermostability: S228P; |
| | | Substitutions for reducing FcγR binding: S228P, L235E, |
| | | P329A |

Construct 120-4

| | | |
|---|---|---|
| SEQ ID NO:7 | SEQ ID NO:187 | IgG1 (IL-12 p40); |
| OR | OR | Heterodimerization mutations: K360E/K409W; |
| SEQ ID | SEQ ID NO:273 | p40 substitution: Y292C (native disulfide bond between |
| NO:238 | | subunits is preserved); |
| | | C220 in the upper hinge is mutated to S |
| SEQ ID NO:8 | SEQ ID NO:188 | IgG1 (IL-12 p35); |
| | | Heterodimerization mutations: Q347R/D399V/F405T; |
| | | Substitution for stabilizing disulfide bond: S354C; |
| | | p35 substitution: V185C (native disulfide bond between |
| | | subunits is preserved); |
| | | C220 in the upper hinge is mutated to S |

Construct 120-5

| | | |
|---|---|---|
| SEQ ID NO:9 | SEQ ID NO:189 | IgG1 (IL-12 p40); |
| OR | OR | Heterodimerization mutations: K360E/K409W; |
| SEQ ID | SEQ ID NO:274 | p40 substitution: Y292C (native disulfide bond between |
| NO:239 | | subunits is preserved); |
| | | Substitutions for reducing FcγR binding: L234A, L235A; |
| | | C220 in the upper hinge is mutated to S |
| SEQ ID | SEQ ID NO:190 | IgG1 (IL-12 p35); |
| NO:10 | | Heterodimerization mutations: Q347R/D399V/F405T; |
| | | p35 substitution: V185C (native disulfide bond between |
| | | subunits is preserved); |
| | | Substitutions for reducing FcγR binding: L234A, L235A; |
| | | C220 in the upper hinge is mutated to S |

Construct 120-6

| | | |
|---|---|---|
| SEQ ID NO:9 | SEQ ID NO:191 | IgG1 (IL-12 p40); |
| OR | OR | Heterodimerization mutations: K360E/K409W; |
| SEQ ID | SEQ ID NO:275 | p40 substitution: Y292C (native disulfide bond between |
| NO:239 | | subunits is preserved); |
| | | Substitutions for reducing FcγR binding: L234A, L235A, |
| | | P329A; |
| | | C220 in the upper hinge is mutated to S |
| SEQ ID | SEQ ID NO:192 | IgG1 (IL-12 p35); |
| NO:10 | | Heterodimerization mutations: Q347R/D399V/F405T; |
| | | p35 substitution: V185C (native disulfide bond between |
| | | subunits is preserved); |
| | | Substitutions for reducing FcγR binding: L234A, L235A, |
| | | P329A; |
| | | C220 in the upper hinge is mutated to S |

Construct 120-7

| | | |
|---|---|---|
| SEQ ID | SEQ ID NO:193 | IgG1 (IL-12 p40); |
| NO:11 | OR | Heterodimerization mutations: K360E/K409W; |
| OR | SEQ ID NO:276 | p40 substitution: Y292C (native disulfide bond between |
| SEQ ID | | subunits is preserved); |
| NO:240 | | Substitutions for reducing FcγR and C1q binding: L234A, |
| | | L235E, G237A, A330S, P331S; |
| | | C220 in the upper hinge is mutated to S |
| SEQ ID | SEQ ID NO:194 | IgG1 (IL-12 p35); |
| NO:12 | | Heterodimerization mutations: Q347R/D399V/F405T; |
| | | p35 substitution: V185C (native disulfide bond between |
| | | subunits is preserved); |
| | | Substitutions for reducing FcγR and C1q binding: L234A, |
| | | L235E, G237A, A330S, P331S; |
| | | C220 in the upper hinge is mutated to S |

Construct 121

| | | |
|---|---|---|
| SEQ ID NO:9 | SEQ ID NO:195 | IgG1 (IL-12 p40); |
| OR | OR | Heterodimerization mutations: K360E/K409W; |
| SEQ ID | SEQ ID NO:277 | Substitution for stabilizing disulfide bond: Y349C; |
| NO:239 | | Substitutions for reducing FcγR binding: L234A, L235A; |
| | | Additional mutation in p40: Y292C to introduce IL-12 |
| | | stabilizing disulfide bond; |
| | | C220 in the upper hinge is mutated to S |
| SEQ ID | SEQ ID NO:196 | IgG1 (IL-12 p35); |
| NO:10 | | Heterodimerization mutations: Q347R/D399V/F405T; |
| | | Substitution for stabilizing disulfide bond: S354C; |
| | | Substitutions for reducing FcγR binding: L234A, L235A; |
| | | Additional mutation in p35: V185C to introduce IL-12 |
| | | stabilizing disulfide bond; |
| | | C220 in the upper hinge is mutated to S |

Construct 122

| | | |
|---|---|---|
| SEQ ID NO:9 | SEQ ID NO:197 | IgG1 (IL-12 p40); |
| OR | OR | Heterodimerization mutations: K360E/K409W; |
| SEQ ID | SEQ ID NO:278 | Substitution for stabilizing disulfide bond: Y349C; |
| NO:239 | | Substitutions for reducing FcγR binding: L234A, L235A, |
| | | P329A; |
| | | Additional mutation Y292C to introduce IL-12 stabilizing |
| | | disulfide bond; |
| | | C220 in the upper hinge is mutated to S |
| SEQ ID | SEQ ID NO:198 | IgG1 (IL-12 p35); |
| NO:10 | | Heterodimerization mutations: Q347R/D399V/F405T; |
| | | Substitution for stabilizing disulfide bond: S354C; |
| | | Substitutions for reducing FcγR binding: L234A, L235A, |
| | | P329A; |
| | | Additional mutation V185C to introduce IL-12 stabilizing |
| | | disulfide bond; |
| | | C220 in the upper hinge is mutated to S |

Construct 123

| | | |
|---|---|---|
| SEQ ID | SEQ ID NO:199 | IgG1 (IL-12 p40); |
| NO:11 | OR | Heterodimerization mutations: K360E/K409W; |
| OR | SEQ ID NO:279 | Substitution for stabilizing disulfide bond: Y349C; |
| SEQ ID | | Substitutions for reducing FcγR and C1q binding: L234A, |
| NO:240 | | L235E, G237A, A330S, P331S; |
| | | Additional mutation Y292C to introduce IL-12 stabilizing |
| | | disulfide bond; |
| | | C220 in the upper hinge is mutated to S |
| SEQ ID | SEQ ID NO:200 | IgG1 (IL-12 p35); |
| NO:12 | | Heterodimerization mutations: Q347R/D399V/F405T; |
| | | Substitution for stabilizing disulfide bond: S354C; |
| | | Substitutions for reducing FcγR and C1q binding: L234A, |
| | | L235E, G237A, A330S, P331S; |
| | | Additional mutation V185C to introduce IL-12 stabilizing |
| | | disulfide bond; |
| | | C220 in the upper hinge is mutated to S |

*The amino acid sequences can further comprise a lysine (K) at the C-terminus.

TABLE 5
Exemplary Dimeric Fc-fused Proteins

| | | |
|---|---|---|
| | Sequences of | |
| | Heterodimeric | Type of IgG (IL-12 subunit); |
| Linker | Fc-fused | mutations/ |
| Sequence | Proteins* | substitutions |

Construct 1

| | | |
|---|---|---|
| SEQ ID NO: 2 | SEQ ID NO: 17 | IgG4 (IL-12 p40); |
| | | Heterodimerization mutations: K370E/R409W; |
| | | Substitution for stabilizing disulfide bond: Y349C; |
| | | Substitution for preventing Fab-arm exchange and improving |
| | | thermostability: S228P |
| SEQ ID NO: 3 | SEQ ID NO: 18 | IgG4 (IL-12 p35); |
| | | Heterodimerization mutations: E357N/D399V/F405T; |
| | | Substitution for stabilizing disulfide bond: S354C; |
| | | Substitution for preventing Fab-arm exchange and improving |
| | | thermostability: S228P |

Construct 2

| | | |
|---|---|---|
| SEQ ID NO: 2 | SEQ ID NO: 19 | IgG4 (IL-12 p40); |
| | | Heterodimerization mutations: K360E/R409W; |
| | | Substitution for stabilizing disulfide bond: Y349C; |
| | | Substitution for preventing Fab-arm exchange and improving |
| | | thermostability: S228P |
| SEQ ID NO: 3 | SEQ ID NO: 20 | IgG4 (IL-12 p35); |
| | | Heterodimerization mutations: Q347R/D399V/F405T; |
| | | Substitution for stabilizing disulfide bond: S354C; |
| | | Substitution for preventing Fab-arm exchange and improving |
| | | thermostability: S228P |

Construct 3

| | | |
|---|---|---|
| SEQ ID NO: 4 | SEQ ID NO: 21 | IgG4 (IL-12 p40); |
| | | Heterodimerization mutations: K360E/R409W; |
| | | Substitution for stabilizing disulfide bond: Y349C; |
| | | Substitution for preventing Fab-arm exchange and improving |
| | | thermostability: S228P; |
| | | Substitutions for reducing FcyR binding: S228P and L235E |
| SEQ ID NO: 5 | SEQ ID NO: 22 | IgG4 (IL-12 p35); |
| | | Heterodimerization mutations: Q347R/D399V/F405T; |
| | | Substitution for stabilizing disulfide bond: S354C; |
| | | Substitution for preventing Fab-arm exchange and improving |
| | | thermostability: S228P; |
| | | Substitutions for reducing FcyR binding: S228P and L235E |

Construct 4

| | | |
|---|---|---|
| SEQ ID NO: 4 | SEQ ID NO: 23 | IgG4 (IL-12 p40); |
| | | Heterodimerization mutations: K360E/R409W; |
| | | Substitution for stabilizing disulfide bond: Y349C; |
| | | Substitution for preventing Fab-Arm exchange and improving |
| | | thermostability: S228P; |
| | | Substitutions for reducing FcyR binding: S228P, L235E, |
| | | P329A |
| SEQ ID NO: 5 | SEQ ID NO: 24 | IgG4 (IL-12 p35); |
| | | Heterodimerization mutations: Q347R/D399V/F405T; |
| | | Substitution for stabilizing disulfide bond: S354C; |
| | | Substitution for preventing Fab-Arm exchange and improving |
| | | thermostability: S228P; |
| | | Substitutions for reducing FcyR binding: S228P, L235E, |
| | | P329A |

Construct 5

| | | |
|---|---|---|
| SEQ ID NO: 4 | SEQ ID NO: 25 | IgG4 (IL-12 p40); |
| | | Heterodimerization mutations: K360E/R409W; |
| | | Substitution for stabilizing disulfide bond: Y349C; |
| | | Substitution for preventing Fab-Arm exchange and improving |
| | | thermostability: S228P; |
| | | Substitutions for reducing FcyR binding: S228P, L235E, |
| | | P329G |
| SEQ ID NO: 5 | SEQ ID NO: 26 | IgG4 (IL-12 p35); |
| | | Heterodimerization mutations: Q347R/D399V/F405T; |
| | | Substitution for stabilizing disulfide bond: S354C, |
| | | Substitution for preventing Fab-Arm exchange and improving |
| | | thermostability: S228P; |
| | | Substitutions for reducing FcyR binding: S228P, L235E, |
| | | P329G |

Construct 6

| | | |
|---|---|---|
| SEQ ID NO: 7 | SEQ ID NO: 27 | IgG1 (IL-12 p40); |
| | | Heterodimerization mutations: K360E/K409W; |
| | | Substitution for stabilizing disulfide bond: Y349C; |
| | | C220 in the upper hinge is mutated to S |
| SEQ ID NO: 8 | SEQ ID NO: 28 | IgG1 (IL-12 p35); |
| | | Heterodimerization mutations: Q347R/D399V/F405T; |
| | | Substitution for stabilizing disulfide bond: S354C; |
| | | C220 in the upper hinge is mutated to S |

Construct 7

| | | |
|---|---|---|
| SEQ ID NO: 9 | SEQ ID NO: 29 | IgG1FcSilent; |
| | | (IL-12 p40); |
| | | Heterodimerization mutations: K360E/K409W; |
| | | Substitution for stabilizing disulfide bond: Y349C; |
| | | Substitutions for reducing FcyR binding: L234A, L235A; |
| | | C220 in the upper hinge is mutated to S |
| SEQ ID NO: 10 | SEQ ID NO: 30 | IgG1FcSilent; |
| | | (IL-12 p35); |
| | | Heterodimerization mutations: Q347R/D399V/F405T; |
| | | Substitution for stabilizing disulfide bond: S354C; |
| | | Substitutions for reducing FcyR binding: L234A, L235A; |
| | | C220 in the upper hinge is mutated to S |

Construct 8

| | | |
|---|---|---|
| SEQ ID NO: 9 | SEQ ID NO: 31 | IgG1FcSilent; |
| | | (IL-12 p40); |
| | | Heterodimerization mutations: K360E/K409W; |
| | | Substitution for stabilizing disulfide bond: Y349C; |
| | | Substitutions for reducing FcyR binding: L234A, L235A, |
| | | P329A; |
| | | C220 in the upper hinge is mutated to S |
| SEQ ID NO: 10 | SEQ ID NO: 32 | IgG1FcSilent; |
| | | (IL-12 p35); |
| | | Heterodimerization mutations: Q347R/D399V/F405T; |
| | | Substitution for stabilizing disulfide bond: S354C; |
| | | Substitutions for reducing FcyR binding: L234A, L235A, |
| | | P329A; |
| | | C220 in the upper hinge is mutated to S |

Construct 9

| | | |
|---|---|---|
| SEQ ID NO: 9 | SEQ ID NO: 33 | IgG1FcSilent; |
| | | (IL-12 p40); |
| | | Heterodimerization mutations: K360E/K409W; |
| | | Substitution for stabilizing disulfide bond: Y349C; |
| | | Substitutions for |
| | | reducing FcyR binding: |
| | | L234A/L235A/P329G; |
| | | C220 in the upper hinge is mutated to S |
| SEQ ID NO: 10 | SEQ ID NO: 34 | IgG1FcSilent; |
| | | (IL-12 p35); |
| | | Heterodimerization mutations: Q347R/D399V/F405T; |
| | | Substitution for stabilizing disulfide bond: S354C; |
| | | Substitutions for reducing FcyR binding: L234A, L235A, |
| | | P329G; |
| | | C220 in the upper hinge is mutated to S |

Construct 10

| | | |
|---|---|---|
| SEQ ID NO: 11 | SEQ ID NO: 35 | IgG1 Fc; |
| | | (IL-12 p40); |
| | | Heterodimerization mutations: K360E/K409W; |
| | | Substitution for stabilizing disulfide bond: Y349C; |
| | | Substitutions for reducing FcyR and Clq binding: L234A, |
| | | L235E, G237A, A330S, P331S |
| SEQ ID NO: 12 | SEQ ID NO: 36 | IgG1 Fc; |
| | | (IL-12 p35); |
| | | Heterodimerization mutations: Q347R/D399V/F405T; |
| | | Substitution for stabilizing disulfide bond: S354C; |
| | | Substitutions for reducing FcyR and Clq binding: L234A, |
| | | L235E, G237A, A330S, P331S |

Construct 11

| | | |
|---|---|---|
| SEQ ID NO: 2 | SEQ ID NO: 37 | IgG4 Fc; |
| | | (IL-12 p40); |
| | | Heterodimerization mutations: K370E/R409W; |
| | | Substitution for stabilizing disulfide bond: Y349C; |
| | | Substitution for preventing Fab-Arm exchange and improving |
| | | thermostability: S228P |
| SEQ ID NO: 2 | SEQ ID NO: 38 | IgG4 Fc; |
| | | (IL-12 p35); |
| | | Heterodimerization mutations: E357N/D399V/F405T; |
| | | Substitution for stabilizing disulfide bond: S354C; |
| | | Substitution for preventing Fab-Arm exchange and improving |
| | | thermostability: S228P |

Construct 12

| | | |
|---|---|---|
| SEQ ID NO: 2 | SEQ ID NO: 39 | IgG4 Fc; |
| | | (IL-12 p40); |
| | | Heterodimerization mutations: K370E/R409W; |
| | | Substitution for stabilizing disulfide bond: Y349C; |
| | | Substitution for preventing Fab-Arm exchange and improving |
| | | thermostability: S228P |
| SEQ ID NO: 13 | SEQ ID NO: 40 | IgG4 Fc; |
| | | (IL-12 p35); |
| | | Heterodimerization mutations: E357N/D399V/F405T; |
| | | Substitution for stabilizing disulfide bond: S354C; |
| | | Substitution for preventing Fab-Arm exchange and improving |
| | | thermostability: S228P |

Construct 13

| | | |
|---|---|---|
| SEQ ID NO: 2 | SEQ ID NO: 41 | IgG4 Fc; |
| | | (IL-12 p40); |
| | | Heterodimerization mutations: K370E/R409W; |
| | | Substitution for stabilizing disulfide bond: Y349C; |
| | | Substitution for preventing Fab-Arm exchange and improving |
| | | thermostability: S228P |
| SEQ ID NO: 14 | SEQ ID NO: 42 | IgG4 Fc; |
| | | (IL-12 p35); |
| | | Heterodimerization mutations: E357N/D399V/F405T; |
| | | Substitution for stabilizing disulfide bond: S354C; |
| | | Substitution for preventing Fab-Arm exchange and improving |
| | | thermostability: S228P |

Construct 14

| | | |
|---|---|---|
| SEQ ID NO: 7 | SEQ ID NO: 43 | IgG1 Fc; |
| | | (IL-12 p40); |
| | | Heterodimerization mutations: K360E/K409W; |
| | | Substitution for stabilizing disulfide bond: Y349C; |
| | | C220 in the upper hinge is mutated to S |
| SEQ ID NO: 7 | SEQ ID NO: 44 | IgG1 Fc; |
| | | (IL-12 p35); |
| | | Heterodimerization mutations: Q347R/D399V/F405T; |
| | | Substitution for stabilizing disulfide bond: S354C; |
| | | C220 in the upper hinge is mutated to S |

Construct 15

| | | |
|---|---|---|
| SEQ ID NO: 7 | SEQ ID NO: 45 | IgG1 Fc; |
| | | (IL-12 p40); |
| | | Heterodimerization mutations: K360E/K409W; |
| | | Substitution for stabilizing disulfide bond: Y349C; |
| | | C220 in the upper hinge is mutated to S |
| SEQ ID NO: 15 | SEQ ID NO: 46 | IgG1 Fc; |
| | | (IL-12 p35); |
| | | Heterodimerization mutations: Q347R/D399V/F405T; |
| | | Substitution for stabilizing disulfide bond: S354C; |
| | | C220 in the upper hinge is mutated to S |

Construct 16

| | | |
|---|---|---|
| SEQ ID NO: 7 | SEQ ID NO: 47 | IgG1 Fc; |
| | | (IL-12 p40); |
| | | Heterodimerization mutations: K360E/K409W; |
| | | Substitution for stabilizing disulfide bond: Y349C; |
| | | C220 in the upper hinge is mutated to S |
| SEQ ID NO: 16 | SEQ ID NO: 48 | IgG1 Fc; |
| | | (IL-12 p35); |
| | | Heterodimerization mutations: Q347R/D399V/F405T; |
| | | Substitution for stabilizing disulfide bond: S354C; |
| | | C220 in the upper hinge is mutated to S |

Construct 17

| | | |
|---|---|---|
| SEQ ID NO: 8 | SEQ ID NO: 49 | IgG1 Fc; |
| | | (IL-12 p35); |
| | | Heterodimerization mutations: K360E/K409W; |
| | | Substitution for stabilizing disulfide bond: Y349C; |
| | | C220 in the upper hinge is mutated to S |
| SEQ ID NO: 7 | SEQ ID NO: 50 | IgG1 Fc; |
| | | (IL-12 p40); |
| | | Heterodimerization mutations: Q347R/D399V/F405T; |
| | | Substitution for stabilizing disulfide bond: S354C; |
| | | C220 in the upper hinge is mutated to S |

Construct 18

| | | |
|---|---|---|
| SEQ ID NO: 15 | SEQ ID NO: 51 | IgG1 (IL-12 p40); |
| | | Heterodimerization mutations: K360E/K409W; |
| | | Substitution for stabilizing disulfide bond: Y349C; |
| | | C220 in the upper hinge is mutated to S |
| SEQ ID NO: 15 | SEQ ID NO: 52 | IgG1 (IL-12 p35); |
| | | Heterodimerization mutations: Q347R/D399V/F405T; |
| | | Substitution for stabilizing disulfide bond: S354C; |
| | | C220 in the upper hinge is mutated to S |

Construct 19

| | | |
|---|---|---|
| SEQ ID NO: 15 | SEQ ID NO: 53 | IgG1 (IL-12 p40); |
| | | Heterodimerization mutations: K360E/K409W; |
| | | p40 substitutions: C177S, Y292C; |
| | | (native disulfide bond between subunits is deleted) |
| SEQ ID NO: 15 | SEQ ID NO: 54 | IgG1 (IL-12 p35); |
| | | Heterodimerization mutations: Q347R/D399V/F405T; |
| | | p35 substitutions: C74S V185C; |
| | | (native disulfide bond between subunits is deleted) |

Construct 19-1

| | | |
|---|---|---|
| SEQ ID NO: 2 | SEQ ID NO: 69 | IgG4 (IL-12 p40); |
| | | Heterodimerization mutations: K370E/R409W; |
| | | p40 substitutions: C177S, Y292C; |
| | | (native disulfide bond between subunits is deleted); |
| | | Substitution for preventing Fab-Arm exchange and improving |
| | | thermostability: S228P |
| SEQ ID NO: 3 | SEQ ID NO: 70 | IgG4 (IL-12 p35); |
| | | Heterodimerization mutations: E357N/D399V/F405T; |
| | | p35 substitutions: C74S V185C; |
| | | Substitution for preventing Fab-Arm exchange and improving |
| | | thermostability: S228P |

Construct 19-2

| | | |
|---|---|---|
| SEQ ID NO: 2 | SEQ ID NO: 71 | IgG4 (IL-12 p40); |
| | | Heterodimerization mutations: K360E/R409W; |
| | | p40 substitutions: C177S, Y292C; |
| | | (native disulfide bond between subunits is deleted); |
| | | Substitution for preventing Fab-Arm exchange and improving |
| | | thermostability: S228P |
| SEQ ID NO: 3 | SEQ ID NO: 72 | IgG4 (IL-12 p35); |
| | | Heterodimerization mutations: Q347R/D399V/F405T; |
| | | p35 substitutions: C74S V185C; |
| | | Substitution for preventing Fab-Arm exchange and improving |
| | | thermostability: S228P |

Construct 19-3

| | | |
|---|---|---|
| SEQ ID NO: 4 | SEQ ID NO: 73 | IgG4 (IL-12 p40); |
| | | Heterodimerization mutations: K360E/R409W; |
| | | p40 substitutions: C177S, Y292C; |
| | | (native disulfide bond between subunits is deleted); |
| | | Substitution for preventing Fab-Arm exchange and improving |
| | | thermostability: S228P; |
| | | Substitutions for reducing FcyR binding: S228P and L235E |
| SEQ ID NO: 5 | SEQ ID NO: 74 | IgG4 (IL-12 p35); |
| | | Heterodimerization mutations: Q347R/D399V/F405T; |
| | | p35 substitutions: C74S V185C; |
| | | Substitution for preventing Fab-Arm exchange and improving |
| | | thermostability: S228P; |
| | | Substitutions for reducing FcyR binding: S228P and L235E |

Construct 19-4

| | | |
|---|---|---|
| SEQ ID NO: 4 | SEQ ID NO: 75 | IgG4 (IL-12 p40); |
| | | Heterodimerization mutations: K360E/R409W; |
| | | p40 substitutions: C177S, Y292C; |
| | | (native disulfide bond between subunits is deleted); |
| | | Substitution for preventing Fab-Arm exchange and improving |
| | | thermostability: S228P; |
| | | Substitutions for reducing FcyR binding: S228P, L235E, |
| | | P329A |
| SEQ ID NO: 5 | SEQ ID NO: 76 | IgG4 (IL-12 p35); |
| | | Heterodimerization mutations: Q347R/D399V/F405T; |
| | | p35 substitutions: C74S V185C; |
| | | Substitution for preventing Fab-Arm exchange and improving |
| | | thermostability: S228P; |
| | | Substitutions for reducing FcyR binding: S228P, L235E, |
| | | P329A |

Construct 19-5

| | | |
|---|---|---|
| SEQ ID NO: 4 | SEQ ID NO: 77 | IgG4 (IL-12 p40); |
| | | Heterodimerization mutations: K360E/R409W; |
| | | p40 substitutions: C177S, Y292C; |
| | | (native disulfide bond between subunits is deleted); |
| | | Substitution for preventing Fab-Arm exchange and improving |
| | | thermostability: S228P; |
| | | Substitutions for reducing FcyR binding: S228P, L235E, |
| | | P329G |
| SEQ ID NO: 5 | SEQ ID NO: 78 | IgG4 (IL-12 p35); |
| | | Heterodimerization mutations: Q347R/D399V/F405T; |
| | | p35 substitutions: C74S V185C; |
| | | Substitution for preventing Fab-Arm exchange and improving |
| | | thermostability: S228P; |
| | | Substitutions for reducing FcyR binding: S228P, L235E, |
| | | P329G |

Construct 19-6

| | | |
|---|---|---|
| SEQ ID NO: 7 | SEQ ID NO: 79 | IgG1 (IL-12 p40); |
| | | Heterodimerization mutations: K360E/K409W; |
| | | p40 substitutions: C177S, Y292C; |
| | | (native disulfide bond between subunits is deleted); |
| | | C220 in the upper hinge is mutated to S |
| SEQ ID NO: 8 | SEQ ID NO: 80 | IgG1 (IL-12 p35); |
| | | Heterodimerization mutations: Q347R/D399V/F405T; |
| | | p35 substitutions: C74S V185C; |
| | | C220 in the upper hinge is mutated to S |

Construct 19-7

| | | |
|---|---|---|
| SEQ ID NO: 9 | SEQ ID NO: 81 | IgG1 (IL-12 p40); |
| | | Heterodimerization mutations: K360E/K409W; |
| | | p40 substitutions: C177S, Y292C; |
| | | (native disulfide bond between subunits is deleted); |
| | | Substitutions for reducing FcyR binding: L234A, L235A; |
| | | C220 in the upper hinge is mutated to S |
| SEQ ID NO: 10 | SEQ ID NO: 82 | IgG1 (IL-12 p35); |
| | | Heterodimerization mutations: Q347R/D399V/F405T; |
| | | p35 substitutions: C74S V185C; |
| | | Substitutions for reducing FcyR binding: L234A, L235A; |
| | | C220 in the upper hinge is mutated to S |

Construct 19-8

| | | |
|---|---|---|
| SEQ ID NO: 9 | SEQ ID NO: 83 | IgG1 (IL-12 p40); |
| | | Heterodimerization mutations: K360E/K409W; |
| | | p40 substitutions: C177S, Y292C; |
| | | (native disulfide bond between subunits is deleted); |
| | | Substitutions for reducing FcyR binding: L234A, L235A, |
| | | P329A; |
| | | C220 in the upper hinge is mutated to S |
| SEQ ID NO: 10 | SEQ ID NO: 84 | IgG1 (IL-12 p35); |
| | | Heterodimerization mutations: Q347R/D399V/F405T; |
| | | p35 substitutions: C74S, V185C; |
| | | Substitutions for reducing FcyR binding: L234A, L235A, |
| | | P329A; |
| | | C220 in the upper hinge is mutated to S |

Construct 19-9

| | | |
|---|---|---|
| SEQ ID NO: 9 | SEQ ID NO: 85 | IgG1 (IL-12 p40); |
| | | Heterodimerization mutations: K360E/K409W; |
| | | p40 substitutions: C177S, Y292C; |
| | | (native disulfide bond between subunits is deleted); |
| | | Substitutions for reducing FcyR binding: |
| | | L234A/L235A/P329G; |
| | | C220 in the upper hinge is mutated to S |
| SEQ ID NO: 10 | SEQ ID NO: 86 | IgG1 (IL-12 p35); |
| | | Heterodimerization mutations: Q347R/D399V/F405T; |
| | | p35 substitutions: C74S V185C; |
| | | Substitutions for reducing FcyR binding: L234A, L235A, |
| | | P329G; |
| | | C220 in the upper hinge is mutated to S |

Construct 19-10

| | | |
|---|---|---|
| SEQ ID NO: 11 | SEQ ID NO: 87 | IgG1 (IL-12 p40); |
| | | Heterodimerization mutations: K360E/K409W; |
| | | p40 substitutions: C177S, Y292C; |
| | | (native disulfide bond between subunits is deleted); |
| | | Substitutions for reducing FcyR and Clq binding: L234A, |
| | | L235E, G237A, A330S, P331S; |
| | | C220 in the upper hinge is mutated to S |
| SEQ ID NO: 12 | SEQ ID NO: 88 | IgG1 (IL-12 p35); |
| | | Heterodimerization mutations: Q347R/D399V/F405T; |
| | | p35 substitutions: C74S, V185C; |
| | | Substitutions for reducing FcyR and Clq binding: L234A, |
| | | L235E, G237A, A330S, P331S; |
| | | C220 in the upper hinge is mutated to S |

Construct 20

| | | |
|---|---|---|
| SEQ ID NO: 2 | SEQ ID NO: 55 | IgG4 (IL-12 p40); |
| | | Heterodimerization mutations: K370E/R409W; |
| | | p40 substitution: Y292C (native disulfide bond between |
| | | subunits is preserved); |
| | | Substitution for preventing Fab-Arm exchange and improving |
| | | thermostability: S228P |
| SEQ ID NO: 3 | SEQ ID NO: 56 | IgG4 (IL-12 p35); |
| | | Heterodimerization mutations: E357N/D399V/F405T; |
| | | p35 substitution: V185C (native disulfide bond between |
| | | subunits is preserved); |
| | | Substitution for preventing Fab-Arm exchange and improving |
| | | thermostability: S228P |

Construct 20-1

| | | |
|---|---|---|
| SEQ ID NO: 2 | SEQ ID NO: 89 | IgG4 (IL-12 p40); |
| | | Heterodimerization mutations: K360E/R409W; |
| | | p40 substitution: Y292C (native disulfide bond between |
| | | subunits is preserved); |
| | | Substitution for preventing Fab-Arm exchange and improving |
| | | thermostability: S228P |
| SEQ ID NO: 3 | SEQ ID NO: 90 | IgG4 (IL-12 p35); |
| | | Heterodimerization mutations: Q347R/D399V/F405T; |
| | | p35 substitution: V185C (native disulfide bond between |
| | | subunits is preserved); |
| | | Substitution for preventing Fab-Arm exchange and improving |
| | | thermostability: S228P |

Construct 20-2

| | | |
|---|---|---|
| SEQ ID NO: 4 | SEQ ID NO: 91 | IgG4 (IL-12 p40); |
| | | Heterodimerization mutations: K360E/R409W; |
| | | p40 substitution: Y292C (native disulfide bond between |
| | | subunits is preserved); |
| | | Substitution for preventing Fab-Arm exchange and improving |
| | | thermostability: S228P; |
| | | Substitutions for reducing FcyR binding: S228P and L235E |
| SEQ ID NO: 5 | SEQ ID NO: 92 | IgG4 (IL-12 p35); |
| | | Heterodimerization mutations: Q347R/D399V/F405T; |
| | | p35 substitution: V185C (native disulfide bond between |
| | | subunits is preserved); |
| | | Substitution for preventing Fab-Arm exchange and improving |
| | | thermostability: S228P; |
| | | Substitutions for reducing FcyR binding: S228P and L235E |

Construct 20-3

| | | |
|---|---|---|
| SEQ ID NO: 4 | SEQ ID NO: 93 | IgG4 (IL-12 p40); |
| | | Heterodimerization mutations: K360E/R409W; |
| | | p40 substitution: Y292C (native disulfide bond between |
| | | subunits is preserved); |
| | | Substitution for preventing Fab-Arm exchange and improving |
| | | thermostability: S228P; |
| | | Substitutions for reducing FcyR binding: S228P, L235E, |
| | | P329A |
| SEQ ID NO: 5 | SEQ ID NO: 94 | IgG4 (IL-12 p35); |
| | | Heterodimerization mutations: Q347R/D399V/F405T; |
| | | p35 substitution: V185C (native disulfide bond between |
| | | subunits is preserved); |
| | | Substitution for preventing Fab-Arm exchange and improving |
| | | thermostability: S228P; |
| | | Substitutions for reducing FcyR binding: S228P, L235E, |
| | | P329A |

Construct 20-4

| | | |
|---|---|---|
| SEQ ID NO: 4 | SEQ ID NO: 95 | IgG4 (IL-12 p40); |
| | | Heterodimerization mutations: K360E/R409W; |
| | | p40 substitution: Y292C (native disulfide bond between |
| | | subunits is preserved); |
| | | Substitution for preventing Fab-Arm exchange and improving |
| | | thermostability: S228P; |
| | | Substitutions for reducing FcyR binding: S228P, L235E, |
| | | P329G |
| SEQ ID NO: 5 | SEQ ID NO: 96 | IgG4 (IL-12 p35); |
| | | Heterodimerization mutations: Q347R/D399V/F405T; |
| | | p35 substitution: V185C (native disulfide bond between |
| | | subunits is preserved); |
| | | Substitution for preventing Fab-Arm exchange and improving |
| | | thermostability: S228P; |
| | | Substitutions for reducing FcyR binding: S228P, L235E, |
| | | P329G |

Construct 20-5

| | | |
|---|---|---|
| SEQ ID NO: 7 | SEQ ID NO: 97 | IgG1 (IL-12 p40); |
| | | Heterodimerization mutations: K360E/K409W; |
| | | p40 substitution: Y292C (native disulfide bond between |
| | | subunits is preserved); |
| | | C220 in the upper hinge is mutated to S |
| SEQ ID NO: 8 | SEQ ID NO: 98 | IgG1 (IL-12 p35); |
| | | Heterodimerization mutations: Q347R/D399V/F405T; |
| | | Substitution for stabilizing disulfide bond: S354C; |
| | | p35 substitution: V185C (native disulfide bond between |
| | | subunits is preserved); |
| | | C220 in the upper hinge is mutated to S |

Construct 20-6

| | | |
|---|---|---|
| SEQ ID NO: 9 | SEQ ID NO: 99 | IgG1 (IL-12 p40); |
| | | Heterodimerization mutations: K360E/K409W; |
| | | p40 substitution: Y292C (native disulfide bond between |
| | | subunits is preserved); |
| | | Substitutions for reducing FcyR binding: L234A, L235A; |
| | | C220 in the upper hinge is mutated to S |
| SEQ ID NO: 10 | SEQ ID | IgG1 (IL-12 p35); |
| | NO: 100 | Heterodimerization mutations: Q347R/D399V/F405T; |
| | | p35 substitution: V185C (native disulfide bond between |
| | | subunits is preserved); |
| | | Substitutions for reducing FcyR binding: L234A, L235A; |
| | | C220 in the upper hinge is mutated to S |

Construct 20-7

| | | |
|---|---|---|
| SEQ ID NO: 9 | SEQ ID | IgG1 (IL-12 p40); |
| | NO: 101 | Heterodimerization mutations: K360E/K409W; |
| | | p40 substitution: Y292C (native disulfide bond between |
| | | subunits is preserved); |
| | | Substitutions for reducing FcyR binding: L234A, L235A, |
| | | P329A; |
| | | C220 in the upper hinge is mutated to S |
| SEQ ID NO: 10 | SEQ ID | IgG1 (IL-12 p35); |
| | NO: 102 | Heterodimerization mutations: Q347R/D399V/F405T; |
| | | p35 substitution: V185C (native disulfide bond between |
| | | subunits is preserved); |
| | | Substitutions for reducing FcyR binding: L234A, L235A, |
| | | P329A; |
| | | C220 in the upper hinge is mutated to S |

Construct 20-8

| | | |
|---|---|---|
| SEQ ID NO: 9 | SEQ ID | IgG1 (IL-12 p40); |
| | NO: 103 | Heterodimerization mutations: K360E/K409W; |
| | | p40 substitution: Y292C (native disulfide bond between |
| | | subunits is preserved); |
| | | Substitutions for reducing FcyR binding: L234A, L235A, |
| | | P329G; |
| | | C220 in the upper hinge is mutated to S |
| SEQ ID NO: 10 | SEQ ID | IgG1 (IL-12 p35); |
| | NO: 104 | Heterodimerization mutations: Q347R/D399V/F405T; |
| | | p35 substitution: V185C (native disulfide bond between |
| | | subunits is preserved); |
| | | Substitutions for reducing FcyR binding: L234A, L235A, |
| | | P329G; |
| | | C220 in the upper hinge is mutated to S |

Construct 20-9

| | | |
|---|---|---|
| SEQ ID NO: 11 | SEQ ID | IgG1 (IL-12 p40); |
| | NO: 105 | Heterodimerization mutations: K360E/K409W; |
| | | p40 substitution: Y292C (native disulfide bond between |
| | | subunits is preserved); |
| | | Substitutions for reducing FcyR and Clq binding: L234A, |
| | | L235E, G237A, A330S, P331S; |
| | | C220 in the upper hinge is mutated to S |
| SEQ ID NO: 12 | SEQ ID | IgG1 (IL-12 p35); |
| | NO: 106 | Heterodimerization mutations: Q347R/D399V/F405T; |
| | | p35 substitution: V185C (native disulfide bond between |
| | | subunits is preserved); |
| | | Substitutions for reducing FcyR and Clq binding: L234A, |
| | | L235E, G237A, A330S, P331S; |
| | | C220 in the upper hinge is mutated to S |

Construct 21

| | | |
|---|---|---|
| SEQ ID NO: 9 | SEQ ID NO: 57 | IgG1 (IL-12 p40); |
| | | Heterodimerization mutations: K360E/K409W; |
| | | Substitution for stabilizing disulfide bond: Y349C; |
| | | Substitutions for reducing FcyR binding: L234A, L235A; |
| | | Additional mutation in p40: Y292C to introduce IL-12 |
| | | stabilizing disulfide bond; |
| | | C220 in the upper hinge is mutated to S |
| SEQ ID NO: 10 | SEQ ID NO: 58 | IgG1 (IL-12 p35); |
| | | Heterodimerization mutations: Q347R/D399V/F405T; |
| | | Substitution for stabilizing disulfide bond: S354C; |
| | | Substitutions for reducing FcyR binding: L234A, L235A; |
| | | Additional mutation in p35: V185C to introduce IL-12 |
| | | stabilizing disulfide bond; |
| | | C220 in the upper hinge is mutated to S |

Construct 22

| | | |
|---|---|---|
| SEQ ID NO: 9 | SEQ ID NO: 59 | IgG1 (IL-12 p40); |
| | | Heterodimerization mutations: K360E/K409W; |
| | | Substitution for stabilizing disulfide bond: Y349C; |
| | | Substitutions for reducing FcyR binding: L234A, L235A, |
| | | P329A; |
| | | Additional mutation Y292C to introduce IL-12 stabilizing |
| | | disulfide bond; |
| | | C220 in the upper hinge is mutated to S |
| SEQ ID NO: 10 | SEQ ID NO: 60 | IgG1 (IL-12 p35); |
| | | Heterodimerization mutations: Q347R/D399V/F405T; |
| | | Substitution for stabilizing disulfide bond: S354C; |
| | | Substitutions for reducing FcyR binding: L234A, L235A, |
| | | P329A; |
| | | Additional mutation V185C to introduce IL-12 stabilizing |
| | | disulfide bond; |
| | | C220 in the upper hinge is mutated to S |

Construct 23

| | | |
|---|---|---|
| SEQ ID NO: 11 | SEQ ID NO: 61 | IgG1 (IL-12 p40); |
| | | Heterodimerization mutations: K360E/K409W; |
| | | Substitution for stabilizing disulfide bond: Y349C; |
| | | Substitutions for reducing FcyR and Clq binding: L234A, |
| | | L235E, G237A, A330S, P331S; |
| | | Additional mutation Y292C to introduce IL-12 stabilizing |
| | | disulfide bond; |
| | | C220 in the upper hinge is mutated to S |
| SEQ ID NO: 12 | SEQ ID NO: 62 | IgG1 (IL-12 p35); |
| | | Heterodimerization mutations: Q347R/D399V/F405T; |
| | | Substitution for stabilizing disulfide bond: S354C; |
| | | Substitutions for reducing FcyR and Clq binding: L234A, |
| | | L235E, G237A, A330S, P331S; |
| | | Additional mutation V185C to introduce IL-12 stabilizing |
| | | disulfide bond; |
| | | C220 in the upper hinge is mutated to S |

*The amino acid sequences can further comprise a lysine (K) at the C-terminus.

(d) IL-12 subunits

IL-12 is a multisubunit protein including a p40 subunit and a p35 subunit. The amino acid sequence of mature wild-type IL-12 p40 is amino acids 23-328 of the GenBank Accession No. NP_002178.2, set forth in SEQ ID NO:127 below. The amino acid sequence of mature wild-type IL-12 p35 is amino acids 57-253 of GenBank Accession No. NP_000873.2, set forth in SEQ ID NO:128 below. The numbering of amino acid residues of p40 and p35 used herein corresponds to the mature wild-type protein sequences. As used herein, an IL-12 p40 subunit comprises an amino acid sequence at least 90% (e.g., at least 91%, 92%, 93%, 94%, 95%, 96%, 97%, 98%, or 99%) identical to SEQ ID NO:127. As used herein, an IL-12 p35 subunit comprises an amino acid sequence at least 90% (e.g., at least 91%, 92%, 93%, 94%, 95%, 96%, 97%, 98%, or 99%) identical to SEQ ID NO:128.

In certain embodiments of any one of the foregoing aspects, the p40 and p35 subunits of IL-12 comprise the amino acid sequences of SEQ ID NOs: 121 and 122; 127 and 128; 201 and 202; 203 and 204; 123 and 124; or 125 and 126, respectively. In certain embodiments, the first polypeptide comprises the amino acid sequence of a p40 subunit of IL-12, and the second polypeptide comprises the amino acid sequence of a p35 subunit of IL-12. In certain embodiments, the first polypeptide comprises the amino acid sequence of a p35 subunit of IL-12, and the second polypeptide comprises the amino acid sequence of a p40 subunit of IL-12.

In certain embodiments, the present disclosure includes a heterodimeric Fc-fused protein comprising: a first polypeptide comprising a first antibody Fc domain polypeptide and a second polypeptide comprising a second antibody Fc domain polypeptide, wherein the first polypeptide further comprises a first subunit of IL-12 fused to the first antibody Fc domain polypeptide by a linker; and a second, different subunit of IL-12 is fused to the second antibody Fc domain polypeptide, wherein the first and second, different subunits of IL-12 are bound to each other, wherein the first antibody Fc domain polypeptide and the second antibody Fc domain polypeptide each contain different mutations promoting heterodimerization, wherein the first antibody Fc domain polypeptide and the second antibody Fc domain polypeptide are bound to each other, and wherein the first subunit of IL-12 is a p40 subunit with a Y292C substitution, and the second, different subunit of IL-12 is a p35 subunit with a V185C substitution. In certain embodiments, the first subunit and second, different subunit of IL-12 comprise the amino acid sequences of SEQ ID NOs: 125 and 126, respectively.

The first subunit and second, different subunit of IL-12 can be fused to any of the antibody Fc domain polypeptides via any linkers disclosed herein to form Fc-fused proteins having sequences including but not limited to Constructs 120, 120-1, 120-2, 120-3, 120-4, 120-5, 120-6, and 120-7 as described in Table 4 and Constructs 20, 20-1, 20-2, 20-3, 20-4, 20-5, 20-6, 20-7, 20-8, and 20-9 as described in Table 5.

In certain embodiments, the p40 subunit of IL-12 further comprises a replacement of C177, and the p35 subunit of IL-12 further comprises a replacement of C74. In certain embodiments, C177 in the p40 subunit of IL-12 is replaced by S, and C74 in the p35 subunit of IL-12 is replaced by S. In certain embodiments, the p40 and p35 subunits of IL-12 comprise the amino acid sequences of SEQ ID NOs: 123 and 124, respectively.

The first subunit and second, different subunit of IL-12 can be fused to any of the antibody Fc domain polypeptides via any linkers disclosed herein to form Fc-fused proteins having sequences including but not limited to Constructs 119, 119-1, 119-2, 119-3, 119-4, 119-5, 119-6, 119-7, and 119-8 as described in Table 4, and Constructs 19, 19-1, 19-2, 19-3, 19-4, 19-5, 19-6, 19-7, 19-8, 19-9, and 19-10 as described in Table 5.

TABLE 6
Human IL-12 p40 and p35 amino acid sequences

| IL-12 p40 | IL-12 p35 |
|---|---|
| Human IL-12 p40 wild-type | Human IL-12 p35 wild-type |
| IWELKKDVYVVELDWYPDAPGEMVVLTCDTPEED | RNLPVATPDPGMFPCLHHSQNLLRAVSNMLQKAR |
| GITWTLDQSSEVLGSGKTLTIQVKEFGDAGQYTC | QTLEFYPCTSEEIDHEDITKDKTSTVEACLPLEL |
| HKGGEVLSHSLLLLHKKEDGIWSTDILKDQKEPK | TKNESCLNSRETSFITNGSCLASRKTSFMMALCL |
| NKTFLRCEAKNYSGRFTCWWLTTISTDLTFSVKS | SSIYEDLKMYQVEFKTMNAKLLMDPKRQIFLDQN |
| SRGSSDPQGVTCGAATLSAERVRGDNKEYEYSVE | MLAVIDELMQALNFNSETVPQKSSLEEPDFYKTK |
| CQEDSACPAAEESLPIEVMVDAVHKLKYENYTSS | IKLCILLHAFRIRAVTIDRVMSYLNAS |
| FFIRDIIKPDPPKNLQLKPLKNSRQVEVSWEYPD | (SEQ ID NO: 128) |
| TWSTPHSYFSLTFCVQVQGKSKREKKDRVFTDKT | |
| SATVICRKNASISVRAQDRYYSSSWSEWASVPCS | |
| (SEQ ID NO: 127) | |
| Human IL-12 p40 C177S Y292C | Human IL-12 p35 C74S V185C |
| SEQ ID NO: 201 | SEQ ID NO: 202 |
| Human IL-12 p40 Y292C | Human IL-12 p35 V185C |
| SEQ ID NO: 203 | SEQ ID NO: 204 |
| Human IL-12 p40 Q56R | Human IL-12 p35 E50V |
| SEQ ID NO: 121 | SEQ ID NO: 122 |
| Human IL-12 p40 Q56R C177S Y292C | Human IL-12 p35 E50V C74S V185C |
| SEQ ID NO: 123 | SEQ ID NO: 124 |
| Human IL-12 p40 Q56R Y292C | Human IL-12 p35 E50V V185C |
| SEQ ID NO: 125 | SEQ ID NO: 126 |

(e) Spacer peptides

Exemplary spacer peptide sequences are provided in Table 7, and exemplary full length linker sequences are provided in Tables 4 and 5.

Within the first polypeptide of the present invention, a first subunit of a multisubunit protein is fused via a linker to a first antibody Fc domain polypeptide (e.g., an IgG4 antibody Fc variant sequence or an IgG1 antibody Fc variant sequence, as disclosed in Table 2), in an amino-to-carboxyl direction. And within the second polypeptide of the present invention, a second, different subunit of a multisubunit protein is fused via a linker to a second antibody Fc domain polypeptide (e.g., an IgG4 antibody Fc variant sequence or an IgG1 antibody Fc variant sequence, as disclosed in Table 2), in an amino-to-carboxyl direction.

In some embodiments, the first subunit of a multisubunit protein of the present invention is fused via a linker to a first antibody Fc domain sequence, wherein the linker comprises or consists of a spacer peptide L₁ and the amino acid sequence of SEQ ID NO:1, 2, 4, 6, 7, 9, 11, 237, 238, 239, or 240. In some embodiments, the second, different subunit of the multisubunit protein is fused to a second antibody Fc domain polypeptide via a linker, wherein the linker comprises or consists of a spacer peptide L2 and the amino acid sequence of SEQ ID NO:1, 2, 4, 6, 7, 9, 11, 237, 238, 239, or 240.

In certain embodiments, L₁ and L₂ are peptide linkers, for example, L₁ and/or L₂ include(s) 4-50 amino acid residues. In certain embodiments, L₁ consists of 4-50 amino acid residues. In certain embodiments, L₁ consists of 4-20 amino acid residues. In certain embodiments, L₂ consists of 4-50 amino acid residues. In certain embodiments, L₂ consists of about 4-20 amino acid residues. In certain embodiments, L₁ and L₂ each independently consist of about 4-50 amino acid residues. In certain embodiments, L₁ and L₂ each independently consist of 4-20 amino acid residues.

In some embodiments, L₁ and L₂ have an optimized length and/or amino acid composition. In some embodiments, L₁ and L₂ are of the same length and have the same amino acid composition. In other embodiments, L₁ and L₂ are different.

In certain embodiments, L₁ is of equal number of amino acids to L₂; in certain embodiments L₁ is longer (i.e., more in the number of amino acids) than L₂; in certain embodiments L₁ is shorter (i.e., fewer number of amino acids) than L₂.

In certain embodiments, L₁ and/or L₂ are “short,” e.g., consist of 0, 1, 2, 3, 4, 5, 6, 7, 8, 9, 10, 11 or 12 amino acid residues. Thus, in certain instances, the spacer peptides consist of about 12 or fewer amino acid residues. In the case of 0 amino acid residues, the spacer peptide is a peptide bond. In certain embodiments, L₁ and/or L₂ are “long,” e.g., consist of 15, 20 or 25 amino acid residues. In some embodiments, the spacer peptides consist of about 3 to about 15, for example 8, 9 or 10 contiguous amino acid residues. Regarding the amino acid composition of L₁ and L₂, peptides are selected with properties that confer flexibility to first and the second polypeptides of the proteins of the present invention, do not interfere with the binding of the first and the second, different subunits to each other, as well as resist cleavage from proteases. For example, glycine and serine residues generally provide protease resistance. The spacer peptides suitable for linking the first subunit of the multisubunit protein to the amino acid sequence of SEQ ID NO:1, 2, 4, 6, 7, 9, 11, 237, 238, 239, or 240, and/or suitable for linking the second, different subunit of the multisubunit protein to the amino acid sequence of SEQ ID NO:1, 2, 4, 6, 7, 9, 11, 237, 238, 239, or 240 may include, as part of a linker, a (GS)ₙ, (GGS)ₙ, (GGGS)ₙ, (GGSG)ₙ, (GGSGG)ₙ, and (GGGGS)ₙ sequence, wherein n is 1, 2, 3, 4, 5, 6, 7, 8, 9, 10, 11, 12, 13, 14, 15, 16, 17, 18, 19, or 20. In some embodiments, L₁ and/or L₂ independently include a (GGGGS)₄ (SEQ ID NO:107) or (GGGGS)₃ (SEQ ID NO:108) sequence as part of a linker. In other embodiments, L₁ and/or L₂ independently include a peptide sequence, as part of a linker, as set forth in the sequences selected from: SEQ ID NO:111, 112, 113, 114, 115, 116, 117, 118, 119, and 120, as listed in Table 7. In some embodiments, L₁ and/or L₂ are independently SEQ ID NO:108, SEQ ID NO:109, or SEQ ID NO:110.

TABLE 7
Linkers

| Linker | SEQ ID NOs |
|---|---|
| G/S Linker | SEQ ID NOs: 111, 112, 113, 114, 115, 116, 117, |
| | 118, 119, and 120 |

In certain embodiments, L₁ includes a sequence, as part of a linker, SEQ ID NO:108, and L₂ includes, as part of a linker, SEQ ID NO:109, or SEQ ID NO:110. In certain embodiments, L₂ includes a sequence, as part of a linker, SEQ ID NO:108, and L₁ includes, as part of a linker, SEQ ID NO:109, or SEQ ID NO:110 sequence. In certain embodiments, L₁, as part of a linker, does not include a sequence as set forth in SEQ ID NO:107, 108, 109, 110, 111, 112, 113, 114, 115, 116, 117, 118, 119, or 120.

In certain embodiments, only L₂, as part of a linker, includes a sequence as set forth in SEQ ID NO:107, 108, 109, 110, 111, 112, 113, 114, 115, 116, 117, 118, 119, or 120. In certain embodiments, neither L₁ nor L₂, as part of a linker sequence, includes a sequence as set forth in SEQ ID NO:107, 108, 109, 110, 111, 112, 113, 114, 115, 116, 117, 118, 119, or 120.

Some heterodimeric Fc-fused proteins of the present invention comprise a first polypeptide comprising a first subunit of a multisubunit protein and a first antibody Fc domain polypeptide, in which a linker comprising SEQ ID NO: 118 connects the first subunit of a multisubunit protein to the first antibody Fc domain polypeptide, for example an Fc domain polypeptide of an IgG4 antibody or an Fc domain polypeptide of an IgG1 antibody. Some heterodimeric Fc-fused proteins of the present invention comprise a second polypeptide comprising a second, different subunit of a multisubunit protein and a second antibody Fc domain polypeptide, in which a linker comprising SEQ ID NO:118 connects the second, different subunit of a multisubunit protein to the second antibody Fc domain polypeptide, for example an Fc domain polypeptide of an IgG4 antibody or an Fc domain polypeptide of an IgG1 antibody.

In certain embodiments, some heterodimeric Fc-fused proteins of the present invention comprise a first polypeptide comprising a first subunit of a multisubunit protein and a first antibody Fc domain polypeptide, in which a linker comprising SEQ ID NO:118; connects the first subunit of a multisubunit protein to the Fc domain polypeptide, and a second polypeptide comprising a second, different subunit of a multisubunit protein and a second antibody Fc domain polypeptide, in which the additional subunit is connected to the second antibody Fc domain polypeptide with a linker that does not comprise SEQ ID NO:118.

In certain embodiments, some heterodimeric Fc-fused proteins of the present invention comprise a first polypeptide comprising a first subunit of a multisubunit protein and a first antibody Fc domain polypeptide, in which a linker that does not comprise SEQ ID NO:118 connects the first subunit of a multisubunit protein to the Fc domain polypeptide, and a second polypeptide comprising a second, different subunit of a multisubunit protein and a second antibody Fc domain polypeptide, in which the second, different subunit of a multisubunit protein is connected to the second antibody Fc domain polypeptide with a linker comprising SEQ ID NO:118.

Some heterodimeric Fc-fused proteins of the present invention comprise a first polypeptide comprising a first subunit of a multisubunit protein and a first antibody Fc domain polypeptide, in which a linker comprising SEQ ID NO: 109 connects the first subunit of a multisubunit protein to the first antibody Fc domain polypeptide, for example an Fc domain polypeptide of an IgG4 antibody or an Fc domain polypeptide of an IgG1 antibody. Some heterodimeric Fc-fused proteins of the present invention comprise a second polypeptide comprising a second, different subunit of a multisubunit protein and a second antibody Fc domain polypeptide, in which a linker comprising SEQ ID NO:109 connects the second, different subunit of a multisubunit protein to the second antibody Fc domain polypeptide, for example an Fc domain polypeptide of an IgG4 antibody or an Fc domain polypeptide of an IgG1 antibody.

Some heterodimeric Fc-fused proteins of the present disclosure include a linker comprising SEQ ID NO:109, which connects a first subunit of a multisubunit protein to a first antibody Fc domain polypeptide, for example an Fc domain polypeptide of an IgG4 antibody or an Fc domain polypeptide of an IgG1 antibody, and connects a second, different subunit of a multisubunit protein to a second antibody Fc domain polypeptide, for example an Fc domain polypeptide of an IgG4 antibody or an Fc domain polypeptide of an IgG1 antibody.

In certain embodiments, some heterodimeric Fc-fused proteins of the present invention comprise a first polypeptide comprising a first subunit of a multisubunit protein and a first antibody Fc domain polypeptide, in which a linker comprising SEQ ID NO:109 connects the first subunit of a multisubunit protein to the Fc domain polypeptide, and a second polypeptide comprising a second, different subunit of a multisubunit protein and a second antibody Fc domain polypeptide, in which additional subunit of a multisubunit protein is connected to the second antibody Fc domain polypeptide with a linker that does not comprise SEQ ID NO:109.

In certain embodiments, some heterodimeric Fc-fused proteins of the present invention comprise a first polypeptide comprising a first subunit of a multisubunit protein and a first antibody Fc domain polypeptide, in which a linker that does not comprise SEQ ID NO:109 connects the first subunit of a multisubunit protein to the Fc domain polypeptide, and a second polypeptide comprising a second, different subunit of a multisubunit protein and a second antibody Fc domain polypeptide, in which the second, different subunit of a multisubunit protein is connected to the second antibody Fc domain polypeptide with a linker comprising SEQ ID NO:109.

Some heterodimeric Fc-fused proteins of the present invention comprise a first polypeptide comprising a first subunit of a multisubunit protein and a first antibody Fc domain polypeptide, in which a linker comprising SEQ ID NO:110 connects the first subunit of a multisubunit protein to the first antibody Fc domain polypeptide, for example an Fc domain polypeptide of an IgG4 antibody or an Fc domain polypeptide of an IgG1 antibody. Some heterodimeric Fc-fused proteins of the present invention comprise a second polypeptide comprising a second, different subunit of a multisubunit protein and a second antibody Fc domain polypeptide, in which a linker comprising SEQ ID NO:110 connects the second, different subunit of a multisubunit protein to the second antibody Fc domain polypeptide, for example an Fc domain polypeptide of an IgG4 antibody or an Fc domain polypeptide of an IgG1 antibody.

Some heterodimeric Fc-fused proteins of the present disclosure include a linker comprising SEQ ID NO:110, which connects a first subunit of a multisubunit protein to a first antibody Fc domain polypeptide, for example an Fc domain polypeptide of an IgG4 antibody or an Fc domain polypeptide of an IgG1 antibody, and connects a second, different subunit of a multisubunit protein to a second antibody Fc domain polypeptide, for example an Fc domain polypeptide of an IgG4 antibody or an Fc domain polypeptide of an IgG1 antibody.

In certain embodiments, some heterodimeric Fc-fused proteins of the present invention comprise a first polypeptide comprising a first subunit of a multisubunit protein and a first antibody Fc domain polypeptide, in which a linker comprising SEQ ID NO:110 connects the first subunit of a multisubunit protein to the Fc domain polypeptide, and a second polypeptide comprising a second, different subunit of a multisubunit protein and a second antibody Fc domain polypeptide, in which the second, different subunit of a multisubunit protein is connected to the second antibody Fc domain polypeptide with a linker that does not comprise SEQ ID NO:110.

In certain embodiments, some heterodimeric Fc-fused proteins of the present invention comprise a first polypeptide comprising a first subunit of a multisubunit protein and a first antibody Fc domain polypeptide, in which a linker that does not comprise SEQ ID NO:110 connects the first subunit of a multisubunit protein to the Fc domain polypeptide, and a second polypeptide comprising a second, different subunit of a multisubunit protein and a second antibody Fc domain polypeptide, in which the second, different subunit of a multisubunit protein is connected to the second antibody Fc domain polypeptide with a linker comprising SEQ ID NO:110.

In certain embodiments, some heterodimeric Fc-fused proteins of the present invention comprise a first polypeptide comprising a first subunit of a multisubunit protein and a first antibody Fc domain polypeptide, in which a linker comprising SEQ ID NO:110 sequence connects the first subunit of a multisubunit protein to the Fc domain polypeptide, and a second polypeptide comprising a second, different subunit of a multisubunit protein and a second antibody Fc domain polypeptide, in which the second, different subunit of a multisubunit protein is connected to the second antibody Fc domain polypeptide with a linker comprising SEQ ID NO:109 sequence.

In certain embodiments, some heterodimeric Fc-fused proteins of the present invention comprise a first polypeptide comprising a first subunit of a multisubunit protein and a first antibody Fc domain polypeptide, in which a linker comprising SEQ ID NO:109 sequence connects the first subunit of a multisubunit protein to the Fc domain polypeptide, and a second polypeptide comprising a second, different subunit of a multisubunit protein and a second antibody Fc domain polypeptide, in which the second, different subunit of a multisubunit protein is connected to the second antibody Fc domain polypeptide with a linker comprising SEQ ID NO:110 sequence.
(f) Fc domain and substitutions for promoting heterodimerization

The assembly of proteins of the present invention can be accomplished by expressing a first polypeptide comprising a first subunit of a multisubunit protein sequence fused to a first antibody Fc domain polypeptide (e.g., an IgG4 antibody Fc variant sequence or an IgG1 antibody Fc variant sequence, as disclosed in Table 2), and a second polypeptide comprising a second, different subunit of a multisubunit protein sequence fused to a second antibody Fc domain polypeptide (e.g., an IgG4 antibody Fc variant sequence or an IgG1 antibody Fc variant sequence, as disclosed in Table 2) in the same cell, which leads to the assembly of a heterodimeric Fc-fused protein according to the invention. The assembled proteins have heterodimeric Fc domain polypeptides with the first antibody Fc domain polypeptide and the second antibody Fc domain polypeptide bound to each other. Promoting the preferential assembly of heterodimers of the Fc can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, U.S. Ser. No. 14/773,418, U.S. Ser. No. 12/811,207, U.S. Ser. No. 13/866,756, U.S. Ser. No. 14/647,480, and U.S. Ser. No. 14/830,336. For example, mutations can be made in the CH3 domain based on human IgG1 and incorporating distinct pairs of amino acid substitutions within a first antibody Fc domain polypeptide and a second antibody Fc domain polypeptide that allow these two chains to selectively heterodimerize with each other. The positions of amino acid substitutions illustrated below are all numbered according to the EU index as in Kabat.

In one scenario, an amino acid substitution in the first antibody Fc domain polypeptide replaces the original amino acid with a larger amino acid, selected from arginine (R), phenylalanine (F), tyrosine (Y) or tryptophan (W), and at least one amino acid substitution in the second antibody Fc domain polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one antibody Fc domain polypeptide can incorporate a T366W substitution, and the other can incorporate three substitutions including T366S, L368A, and Y407V.

A first polypeptide comprising a first subunit of a multisubunit protein sequence or a second polypeptide comprising a second, different subunit of a multisubunit protein sequence of the invention can optionally be coupled to an amino acid sequence at least 90% (e.g., at least 91%, 92%, 93%, 94%, 95%, 96%, 97%, 98%, or 99%) identical to an antibody constant region, such as an IgG constant region including hinge, CH2 and CH3 domains with or without a CH1 domain. In some embodiments, the amino acid sequence of the constant region is at least 90% (e.g., at least 91%, 92%, 93%, 94%, 95%, 96%, 97%, 98%, or 99%) identical to a human antibody constant region, such as a human IgG1 constant region, an IgG2 constant region, an IgG3 constant region, or an IgG4 constant region. In some other embodiments, the amino acid sequence of the constant region is at least 90% (e.g., at least 91%, 92%, 93%, 94%, 95%, 96%, 97%, 98%, or 99%) identical to an antibody constant region from another mammal, such as rabbit, dog, cat, mouse, or horse. One or more mutation(s) can be incorporated into the constant region as compared to the human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, T411 D, T411E, K439D, and K439E.

In certain embodiments, mutations that can be incorporated into the CH1 of a human IgG1 constant region may be at amino acids V125, F126, P127, T135, T139, A140, F170, P171, and/or V173. In certain embodiments, mutations that can be incorporated into the Cx of a human IgG1 constant region may be at amino acids E123, F116, S176, V163, S174, and/or T164.

Amino acid substitutions could be selected from the following sets of substitutions shown in Table 8.

TABLE 8
Amino Acid Substitutions

| | First | Second |
|---|---|---|
| | Polypeptide | Polypeptide |
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 9.

TABLE 9
Amino Acid Substitutions

| | First Polypeptide | Second Polypeptide |
|---|---|---|
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following set of substitutions shown in Table 10.

TABLE 10
Amino Acid Substitutions

| | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 11.

TABLE 11
Amino Acid Substitutions

| First Polypeptide | Second Polypeptide |
|---|---|
| L351Y, D399R, D399K, S400K, | T366V, T366I, T366L, T366M, N390D, N390E, |
| S400R, Y407A, Y4071, Y407V | K392L, K392M, K392V, K392F K392D, K392E, |
| | K409F, K409W, T411D and T411E |

Alternatively, at least one amino acid substitutions could be selected from the following set of substitutions in Table 12, where the position(s) indicated in the First Polypeptide column is replaced by any known negatively-charged amino acid, and the position(s) indicated in the Second Polypeptide Column is replaced by any known positively-charged amino acid.

TABLE 12
Amino Acid Substitutions

| First Polypeptide | Second Polypeptide |
|---|---|
| K392, K370, K409, or K439 | D399, E356, or E357 |

Alternatively, at least one amino acid substitutions could be selected from the following set of in Table 13, where the position(s) indicated in the First Polypeptide column is replaced by any known positively-charged amino acid, and the position(s) indicated in the Second Polypeptide Column is replaced by any known negatively-charged amino acid.

TABLE 13
Amino Acid Substitutions

| First Polypeptide | Second Polypeptide |
|---|---|
| D399, E356, or E357 | K409, K439, K370, or K392 |

Alternatively, amino acid substitutions could be selected from the following set in Table 14.

TABLE 14
Amino Acid Substitutions

| First Polypeptide | Second Polypeptide |
|---|---|
| T350V, L351Y, F405A, | T350V, T366L, K392L, |
| and Y407V | and T394W |

Alternatively, or in addition, the structural stability of a heterodimeric Fc-fused protein according to the invention may be increased by introducing S354C on either of the first or second polypeptide chain, and Y349C on the opposing polypeptide chain, which forms an artificial disulfide bond within the interface of the two polypeptides.

In some embodiments, the amino acid sequence of one polypeptide chain of the antibody constant region differs from the amino acid sequence of an IgG1 constant region at position T366, and the amino acid sequence of the other polypeptide chain of the antibody constant region differs from the amino acid sequence of an IgG1 constant region at one or more position(s) selected from T366, L368 and Y407.

In some embodiments, the amino acid sequence of one polypeptide chain of the antibody constant region differs from the amino acid sequence of an IgG1 constant region at one or more position(s) selected from T366, L368 and Y407, and the amino acid sequence of the other polypeptide chain of the antibody constant region differs from the amino acid sequence of an IgG1 constant region at position T366.

In some embodiments, the amino acid sequence of one polypeptide chain of the antibody constant region differs from the amino acid sequence of an IgG1 constant region at one or more position(s) selected from E357, K360, Q362, S364, L368, K370, T394, D401, F405, and T411 and the amino acid sequence of the other polypeptide chain of the antibody constant region differs from the amino acid sequence of an IgG1 constant region at one or more position(s) selected from Y349, E357, S364, L368, K370, T394, D401, F405 and T411.

In some embodiments, the amino acid sequence of one polypeptide chain of the antibody constant region differs from the amino acid sequence of an IgG1 constant region at one or more position(s) selected from Y349, E357, S364, L368, K370, T394, D401, F405 and T411 and the amino acid sequence of the other polypeptide chain of the antibody constant region differs from the amino acid sequence of an IgG1 constant region at one or more position(s) selected from E357, K360, Q362, S364, L368, K370, T394, D401, F405, and T411.

In some embodiments, the amino acid sequence of one polypeptide chain of the antibody constant region differs from the amino acid sequence of an IgG1 constant region at one or more position(s) selected from L351, D399, S400 and Y407 and the amino acid sequence of the other polypeptide chain of the antibody constant region differs from the amino acid sequence of an IgG1 constant region at one or more position(s) selected from T366, N390, K392, K409 and T411.

In some embodiments, the amino acid sequence of one polypeptide chain of the antibody constant region differs from the amino acid sequence of an IgG1 constant region at one or more position(s) selected from T366, N390, K392, K409 and T411 and the amino acid sequence of the other polypeptide chain of the antibody constant region differs from the amino acid sequence of an IgG1 constant region at one or more position(s) selected from L351, D399, S400 and Y407.

In some embodiments, the amino acid sequence of one polypeptide chain of the antibody constant region differs from the amino acid sequence of an IgG1 constant region at one or more position(s) selected from Q347, Y349, K360, and K409, and the amino acid sequence of the other polypeptide chain of the antibody constant region differs from the amino acid sequence of an IgG1 constant region at one or more position(s) selected from Q347, E357, D399 and F405.

In some embodiments, the amino acid sequence of one polypeptide chain of the antibody constant region differs from the amino acid sequence of an IgG1 constant region at one or more position(s) selected from Q347, E357, D399 and F405, and the amino acid sequence of the other polypeptide chain of the antibody constant region differs from the amino acid sequence of an IgG1 constant region at one or more position(s) selected from Y349, K360, Q347 and K409.

In some embodiments, the amino acid sequence of one polypeptide chain of the antibody constant region differs from the amino acid sequence of an IgG1 constant region at one or more position(s) selected from K370, K392, K409 and K439, and the amino acid sequence of the other polypeptide chain of the antibody constant region differs from the amino acid sequence of an IgG1 constant region at one or more position(s) selected from D356, E357 and D399.

In some embodiments, the amino acid sequence of one polypeptide chain of the antibody constant region differs from the amino acid sequence of an IgG1 constant region at one or more position(s) selected from D356, E357 and D399, and the amino acid sequence of the other polypeptide chain of the antibody constant region differs from the amino acid sequence of an IgG1 constant region at one or more position(s) selected from K370, K392, K409 and K439.

In some embodiments, the amino acid sequence of one polypeptide chain of the antibody constant region differs from the amino acid sequence of an IgG1 constant region at one or more position(s) selected from L351, E356, T366 and D399, and the amino acid sequence of the other polypeptide chain of the antibody constant region differs from the amino acid sequence of an IgG1 constant region at one or more position(s) selected from Y349, L351, L368, K392 and K409.

In some embodiments, the amino acid sequence of one polypeptide chain of the antibody constant region differs from the amino acid sequence of an IgG1 constant region at one or more position(s) selected from Y349, L351, L368, K392 and K409, and the amino acid sequence of the other polypeptide chain of the antibody constant region differs from the amino acid sequence of an IgG1 constant region at one or more position(s) selected from L351, E356, T366 and D399.

In some embodiments, the amino acid sequence of one polypeptide chain of the antibody constant region differs from the amino acid sequence of an IgG1 constant region by an S354C substitution and the amino acid sequence of the other polypeptide chain of the antibody constant region differs from the amino acid sequence of an IgG1 constant region by a Y349C substitution.

In some embodiments, the amino acid sequence of one polypeptide chain of the antibody constant region differs from the amino acid sequence of an IgG1 constant region by a Y349C substitution and the amino acid sequence of the other polypeptide chain of the antibody constant region differs from the amino acid sequence of an IgG1 constant region by an S354C substitution.

In some embodiments, the amino acid sequence of one polypeptide chain of the antibody constant region differs from the amino acid sequence of an IgG1 constant region by K360E and K409W substitutions and the amino acid sequence of the other polypeptide chain of the antibody constant region differs from the amino acid sequence of an IgG1 constant region by 0347R, D399V and F405T substitutions.

In some embodiments, the amino acid sequence of one polypeptide chain of the antibody constant region differs from the amino acid sequence of an IgG1 constant region by 0347R, D399V and F405T substitutions and the amino acid sequence of the other polypeptide chain of the antibody constant region differs from the amino acid sequence of an IgG1 constant region by K360E and K409W substitutions.

In some embodiments, the amino acid sequence of one polypeptide chain of the antibody constant region differs from the amino acid sequence of an IgG1 constant region by a T366W substitutions and the amino acid sequence of the other polypeptide chain of the antibody constant region differs from the amino acid sequence of an IgG1 constant region by T366S, T368A, and Y407V substitutions.

In some embodiments, the amino acid sequence of one polypeptide chain of the antibody constant region differs from the amino acid sequence of an IgG1 constant region by T366S, T368A, and Y407V substitutions and the amino acid sequence of the other polypeptide chain of the antibody constant region differs from the amino acid sequence of an IgG1 constant region by a T366W substitution.

In some embodiments, the amino acid sequence of one polypeptide chain of the antibody constant region differs from the amino acid sequence of an IgG1 constant region by T350V, L351Y, F405A, and Y407V substitutions and the amino acid sequence of the other polypeptide chain of the antibody constant region differs from the amino acid sequence of an IgG1 constant region by T350V, T366L, K392L, and T394W substitutions.

In some embodiments, the amino acid sequence of one polypeptide chain of the antibody constant region differs from the amino acid sequence of an IgG1 constant region by T350V, T366L, K392L, and T394W substitutions and the amino acid sequence of the other polypeptide chain of the antibody constant region differs from the amino acid sequence of an IgG1 constant region by T350V, L351Y, F405A, and Y407V substitutions.

A skilled person in the art would appreciate that during production and/or storage of proteins, N-terminal glutamate (E) or glutamine (Q) can be cyclized to form a lactam (e.g., spontaneously or catalyzed by an enzyme present during production and/or storage). Accordingly, in some embodiments where the N-terminal residue of an amino acid sequence of a polypeptide is E or Q, a corresponding amino acid sequence with the E or Q replaced with pyroglutamate is also contemplated herein.

A skilled person in the art would also appreciate that during protein production and/or storage, the C-terminal lysine (K) of a protein can be removed (e.g., spontaneously or catalyzed by an enzyme present during production and/or storage). Such removal of K is often observed with proteins that comprise a Fc domain at its C-terminus. Accordingly, in some embodiments where the C-terminal residue of an amino acid sequence of a polypeptide (e.g., a Fc domain sequence) is K, a corresponding amino acid sequence with the K removed is also contemplated herein.
(g) Mutations for reducing effector functions

In one aspect, the present invention provides a heterodimeric Fc-fused protein comprising (a) a first polypeptide comprising a first antibody Fc domain polypeptide and a first subunit of a multisubunit protein; and (b) a second polypeptide comprising a second antibody Fc domain polypeptide and a second, different subunit of the multisubunit protein, wherein the first and second antibody Fc domain polypeptides each comprise different mutations promoting heterodimerization, wherein the first and/or second antibody Fc domain polypeptides comprise one or more mutation(s) that reduce(s) an effector function of an Fc, and wherein the first subunit and second, different subunit of the multisubunit protein are bound to each other. In certain embodiments, a heterodimeric Fc-fused protein disclosed herein comprising one or more mutation(s) that reduce(s) an effector function of an Fc has an increased activity to inhibit tumor growth than its counterpart without the Fc mutation(s) that reduce(s) the effector function. The mutations contemplated herein include substitution, insertion, and deletion of amino acid residues. All the amino acid positions in an Fc domain or hinge region disclosed herein are numbered according to EU numbering.

In certain embodiments, the first and/or second antibody Fc domain polypeptides comprise one or more mutation(s) that reduce(s) the ability of the Fc domain polypeptide to induce antibody-dependent cellular cytotoxicity (ADCC) and/or antibody-dependent cellular phagocytosis (ADCP). ADCC and ADCP are typically mediated by an Fc receptor. For example, in certain embodiments, the first and second antibody Fc domain polypeptides are human IgG (e.g., human IgG1, human IgG2, human IgG3, or human IgG4) antibody sequences. The Fc receptors of human IgG, also called Fc gamma receptors (FcγRs), include but are not limited to activating Fc gamma receptors FcγRI (CD64), FcγRIIA (CD32A), FcγRIIIA (CD16 or CD16A), and FcγRIIIB (CD16B), and inhibitor Fc gamma receptor FcγRIIB (CD32B). Accordingly, in some embodiments, a heterodimeric Fc-fused protein of the present invention includes one or more mutation(s) to reduce binding to an activating FcγR (e.g., FcγRI, FcγRIIA, FcγRIIIA, or FcγRIIIB) in the first and/or second polypeptides. In some embodiments, a heterodimeric Fc-fused protein of the present invention includes one or more mutation(s) to increase binding to an inhibitory FcγR (e.g., FcγRIIB) in the first and/or second polypeptides.

Fc mutations that reduce binding to an activating FcγR and/or increase binding to an inhibitory FcγR are known in the art. For example, within the hinge and Fc regions, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al, Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

As reviewed in Want et al., Protein Cell (2018) 9(1):63-73, the regions including amino acid positions 232-239, 265-270, 296-299, and 325-332 are implicated in activating FcγR binding according to a crystal structure of human IgG1 Fc. Wang et al. also discloses that L235E and F234A/L235A mutations of human IgG4, L234A/L235A mutations of human IgG1, and N297 mutations (e.g., N297A, N297Q, N297G, or N297D) of IgG antibodies reduce activating FcγR binding. As disclosed in U.S. Pat. No. 8,969,526, mutation at position 329 (e.g., P329A, P329G, or P329R) also reduces activating FcγR binding. Additional amino acid positions and mutations (e.g., E233P mutation) implicated in activating FcγR binding are disclosed in U.S. Pat. No. 7,943,743 and Isaacs et al., J. Immunol. (1998) 161:3862-69.

Accordingly, in certain embodiments, the first and second antibody Fc domain polypeptides comprise a mutation (e.g., substitution relative to wild-type human IgG1) at one or more of positions selected from 233, 234, 235, 297, and 329. In certain embodiments, the first and second antibody Fc domain polypeptides are human IgG1 antibody Fc domain polypeptides comprising mutation(s) E233P; L234A (human IgG1) or F234A (human IgG4); L235A or L235E; N297A, N297Q, N297G, or N297D; and/or P329A, P329G, or P329R. In certain embodiments, the first and second antibody Fc domain polypeptides are human IgG1 antibody Fc domain polypeptides comprising mutations L234A and L235A. In certain embodiments, the first and second antibody Fc domain polypeptides are human IgG1 antibody Fc domain polypeptides comprising mutations L234A, L235A, and P329A. In certain embodiments, the first and second antibody Fc domain polypeptides are human IgG4 antibody Fc domain polypeptides comprising mutation L235E. In certain embodiments, the first and second antibody Fc domain polypeptides are human IgG1 antibody Fc domain polypeptides comprising mutations L235E and P329A.

In certain embodiments, the first and/or second antibody Fc domain polypeptides comprise one or more mutation(s) that reduce(s) the ability of the Fc domain polypeptide to induce complement dependent cytotoxicity (CDC). CDC is typically mediated by a complement component (e.g., C1q). Accordingly, in certain embodiments, a heterodimeric Fc-fused protein of the present invention includes one or more mutation(s) to reduce binding to a complement component (e.g., C1q) in the first and/or second polypeptides.

Fc mutations that reduce binding to C1q are known in the art. For example, as disclosed in U.S. Pat. Nos. 5,648,260 and 5,624,821, the amino acid residues of Fc at positions 234, 235, 236, 237, 297, 318, 320, and 322 are implicated in C1q binding. As disclosed in Tao et al., J. Exp. Med. (1993) 178:661-667 and Brekke et al., Eur. J. Immunol. (1994) 24:2542-47, residue Pro at position 331 is implicated in C1q binding. As disclosed in Idusogie et al., J. Immunol. (2000) 164:4178-84, mutations of Fc at positions 270 (e.g., D270A), 322 (K322A), 329 (e.g., P329A), and 331 (e.g., P331A, P331S, or P331G) reduced C1q binding.

Accordingly, in certain embodiments, the first and second antibody Fc domain polypeptides comprise a mutation (e.g., substitution relative to wild-type human IgG1) at one or more of positions selected from 234, 235, 236, 237, 270, 297, 318, 320, 322, 329, and 331. In certain embodiments, the first and second antibody Fc domain polypeptides are human IgG1 antibody Fc domain polypeptides comprising mutation(s) G237A, A330S, P331S, and/or P329A. In certain embodiments, the first and second antibody Fc domain polypeptides are human IgG1 antibody Fc domain polypeptides comprising mutations G237A, A330S, and P331S. In certain embodiments, the first and second antibody Fc domain polypeptides are human IgG1 antibody Fc domain polypeptides comprising mutation P329A.

The mutations that reduce ADCC and/or ADCP and the mutations that reduce CDC can be combined. In certain embodiments, the first and/or second antibody Fc domain polypeptides comprise one or more mutation(s) that reduce(s) the ability of the Fc domain polypeptide to induce ADCC and/or ADCP and further comprise one or more mutation(s) that reduce(s) the ability of the Fc domain polypeptide to induce CDC. In certain embodiments, the first and second antibody Fc domain polypeptides each comprise one or more mutation(s) that reduce(s) the ability of the Fc domain polypeptide to induce ADCC and/or ADCP and further comprise one or more mutation(s) that reduce(s) the ability of the Fc domain polypeptide to induce CDC.

In some embodiments, a heterodimeric Fc-fused protein of the present invention with an IgG4 Fc includes one or more mutation(s) to reduce binding to an FcγR (e.g., FcγRI, FcγRIIA, FcγRIIB, FcγRIIIA, or FcγRIIIB) or a complement component (e.g., C1q) in the first and/or second polypeptides. Such mutations are useful for reducing effector functions. For example, a protein of the present disclosure can include S228P and L235E mutations; S228P, L235E, and P329A mutations; or S228P, L235E, and P329G mutations.

In some embodiments, a heterodimeric Fc-fused protein of the present invention with an IgG1 Fc includes one or more mutation(s) to reduce binding to an FcγR (e.g., FcγRI, FcγRIIA, FcγRIIB, FcγRIIIA, or FcγRIIIB) or a complement component (e.g., C1q) in the first and/or second polypeptides. Such mutations are useful for reducing effector functions. For example, a protein of the present disclosure can include L234A and L235A mutations; L234A, L235A, and P329A mutations; L234A, L235A, and P329G mutations; or L234A, L235E, G237A, A330S, and P331S mutations.

In some embodiments, a heterodimeric Fc-fused protein according to the invention includes a first antibody IgG4 or IgG1 Fc domain polypeptide and a second antibody IgG4 or IgG1 Fe domain polypeptide each containing the mutation P329G or P329A.

In some embodiments, the first antibody Fe domain polypeptide and the second antibody Fe domain polypeptide each contain a mutation selected from A330S and P331S.

In some embodiments, the first antibody Fe domain polypeptide and the second antibody Fe domain polypeptide each contain the mutations A330S and P331S.

In certain embodiments, in the first polypeptide of the heterodimeric Fc-fused protein of the present invention, the first subunit of the multisubunit protein is fused to the first antibody Fe domain polypeptide by a first linker. In certain embodiments, in the second polypeptide of the heterodimeric Fc-fused protein of the present invention, the second, different subunit of the multisubunit protein is fused to the second antibody Fe domain polypeptide by a second linker. Amino acid sequences of linkers suitable for such use are described under the headings “IgG4 constructs” and “IgG1 constructs.” Additional linker sequences suitable for use in the first and/or second polypeptides include but are not limited to wild-type IgG (e.g., human IgG1, human IgG2, human IgG3, or human IgG4) hinge sequences and mutant forms thereof. For example, in certain embodiments, the first and second linkers each comprise amino acid sequence ESKYGPPCPPCPAPEFXGG, wherein X is L or E (SEQ ID NO:280) or SKYGPPCPPCPAPEFXGG, wherein X is L or E (SEQ ID NO:281). In certain embodiments, the first and second linkers each comprise amino acid sequence of SEQ ID NO:282 or of SEQ ID NO:283. In certain embodiments, the first and second linkers each comprise amino acid sequence of SEQ ID NO:284 or of SEQ ID NO:285.
(h) Serum Half-life

Heterodimeric Fc-fused proteins according to the invention have pharmacokinetic properties suitable for therapeutic use. For example, in certain embodiments, a heterodimeric Fc-fused protein according to the invention has a serum half-life of at least about 50 hours. In certain embodiments, a heterodimeric Fc-fused protein according to the invention has a serum half-life of at least about 100 hours.

In certain embodiments, 50 hours after intravenous administration to a subject, the serum concentration of the heterodimeric Fc-fused protein according to the invention is at least 10% of the serum concentration of the protein of the present invention 1 hour after the administration in said subject.

In certain embodiments, a heterodimeric Fc-fused protein according to the invention has a serum half-life that is at least 20%, 30%, 40%, 50%, 60%, 70%, 80%, or 90% longer than the multisubunit protein not fused to Fc domain polypeptides. In certain embodiments, a heterodimeric Fc-fused protein comprising a protein sequence of a multisubunit protein according to the present invention has a serum half-life that is at least 2-fold, 3-fold, 4-fold, 5-fold, 6-fold, 7-fold, 8-fold, 9-fold, 10-fold, 15-fold, or 20-fold longer than the multisubunit protein not fused to Fc domain polypeptides.
(i) Tumor Retention

Heterodimeric Fc-fused proteins of the invention can optionally incorporate additional features to enhance retention of the proteins at the tumor site. For example, in certain embodiments of the present invention, the heterodimeric Fc-fused protein further comprises a proteoglycan-binding domain, a collagen-binding domain, and/or a hyaluronic acid-binding domain. In certain embodiments, the heterodimeric Fc-fused protein further comprises a proteoglycan-binding domain that binds one or more proteoglycans (e.g., proteoglycans known in the art, e.g., as disclosed in Lozzo et al., Matrix Bio (2015) 42:11-55; and Nikitovic et al., Frontiers in Endocrinology (2018) 9:69) that are present in a tumor (e.g., on the surface of a tumor cell, in a pericellular matrix in a tumor, or in a extracellular matrix in a tumor). In certain embodiments, the collagen-binding domain binds one or more collagens that are present in a tumor (e.g., on the surface of a tumor cell, in a pericellular matrix in a tumor, or in a extracellular matrix in a tumor). In certain embodiments, the heterodimeric Fc-fused protein further comprises a h acid-binding domain that binds to one or more hyaluronic acid that are present in a tumor. Such heterodimeric Fc-fused proteins have enhanced retention in tumors and may be administered to a subject intratumorally at a lower dose and/or frequency.

In certain embodiments, the proteoglycan-binding domain comprised in the heterodimeric Fc-fused protein binds one or more proteoglycans that are specifically expressed in a tumor (e.g., on the surface of a tumor cell, in a pericellular matrix in a tumor, or in a extracellular matrix in a tumor). In certain embodiments, the collagen-binding domain comprised in the heterodimeric Fc-fused protein binds one or more collagens that are specifically expressed in a tumor (e.g., on the surface of a tumor cell, in a pericellular matrix in a tumor, or in a extracellular matrix in a tumor). Such heterodimeric Fc-fused proteins may be enriched in tumors after administration (e.g., intravenous, subcutaneous, or pulmonary administration) and have enhanced tumor retention, thereby allowing administration at a lower dose and/or frequency.

In certain embodiments, the heterodimeric Fc-fused protein of the present invention further comprises a proteoglycan-binding domain that binds one or more proteoglycans selected from syndecan, chondroitin sulfate proteoglycan 4 (CSPG4), betaglycan, phosphacan, glypican, perlecan, agrin, collagen (e.g., collagen IX, XII, XV, or XVIII), hyalectan, aggrecan, versican, neurocan, brevican, and a small leucine-rich proteoglycan (SLRP). Proteoglycans implicated in cancer include but are not limited to collagen, syndecan (e.g., syndecan-1 or syndecan-2), serglycin, CSPG4, betaglycan, glypican (e.g., glypican-1 or glypican-3), perlecan, versican, brevican, and SLPR (e.g., decorin, biglycan, asporin, fibrodulin, and lumican). Accordingly, in certain embodiments, the proteoglycan-binding domain comprised in the heterodimeric Fc-fused protein binds one or more proteoglycans selected from syndecan (e.g., syndecan-1 or syndecan-2), serglycin, CSPG4, betaglycan, glypican (e.g., glypican-1 or glypican-3), perlecan, versican, brevican, and a SLPR. In certain embodiments, the proteoglycan-binding domain comprised in the heterodimeric Fc-fused protein binds one or more SLPRs selected from decorin, biglycan, asporin, fibrodulin, and lumican.

The proteoglycan-binding domain comprised in the heterodimeric Fc-fused protein can be a protein (e.g., an antibody or an antigen-binding fragment thereof), a peptide (e.g., a portion of a proteoglycan-binding protein or a variant thereof), an aptamer, a small molecule, or a combination thereof. Proteoglycan-binding domains are also known in the art. For example, syndecan-binding domains are disclosed in U.S. Pat. Nos. 6,566,489, 8,647,828, and 10,124,038; U.S. Patent Application Publication No. 2009/0297479; and PCT Patent Application Publication No. WO2018199176A1. CSPG4-binding domains are disclosed in U.S. Pat. Nos. 9,801,928 and 10,093,745; and U.S. Patent Application Publication Nos. 2016/0032007, 2017/0342151, and 2018/0072811. β-glycan-binding domains are disclosed in U.S. Pat. No. 7,455,839. Glypican-binding domains are disclosed in U.S. Pat. Nos. 7,919,086, 7,776,329, 8,680,247, 8,388,937, 9,260,492, 9,394,364, 9,790,267, 9,522,940, and 9,409,994; U.S. Patent Application Publication Nos. 2004/0236080, 2011/0123998, 2018/0244805, 2018/0230230, and 2018/0346592; European Patent No. 2270509; and PCT Patent Application Publication No. WO2017053619A1, WO2018026533A1, WO2018165344A1, and WO2018199318A1. Perlecan-binding domains are disclosed in U.S. Pat. No. 10,166,304. Decorin-binding domains are disclosed in U.S. Pat. No. 6,517,838 and PCT Patent Application Publication No. WO2000021989A1, WO2000077041A2, and WO2000078800A2.

In certain embodiments, the heterodimeric Fc-fused protein of the present invention further comprises a collagen-binding domain. Collagen is a class of proteins having at least 28 different types identified in vertebrates. Each type of collagen has its unique structural characteristics and distribution pattern, as disclosed in Fang et al., Tumor Biol. (2014) 35:2871-82 and Xiong et al., J. Cancer Metasta. Treat. (2016) 2:357-64. Various types of collagens are implicated in cancer, including but not limited to Col3A1, Col5A2, Col6, Col7A1, Col15A1 Col19A1, and Col22A1. The collagen-binding domain can be a protein (e.g., an antibody or an antigen-binding fragment thereof), a peptide (e.g., a portion of a collagen-binding protein or a variant thereof), an aptamer, a small molecule, or a combination thereof. Collagen-binding domains are known in the art, and are disclosed in, for example, U.S. Pat. Nos. 5,788,966, 5,587,360, 5,851,794, 5,741,670, 5,849,701, 6,288,214, 6,387,663, 6,908,994, 7,169,902, 7,488,792, 7,820,401, 8,956,612, 8,642,728, and 8,906,649, and U.S. Patent Application Publication Nos. 2007/0161062, 2009/0142345, and 2012/0100106.

In certain embodiments, the heterodimeric Fc-fused protein of the present invention further comprises a hyaluronic acid-binding domain. The hyaluronic acid-binding domain can be a protein (e.g., an antibody or an antigen-binding fragment thereof), a peptide (e.g., a portion of a hyaluronic acid-binding protein or a variant thereof), an aptamer, a small molecule, or a combination thereof. Hyaluronic acid-binding domains are known in the art, and are disclosed in, for example, U.S. Pat. Nos. 6,864,235, 8,192,744, 8,044,022, 8,163,498, 8,034,630, 9,217,016, 9,795,686, and 9,751,919, and U.S. Patent Application Publication Nos. 2002/0055488 and 2007/0259380.

A proteoglycan-binding domain, collagen-binding domain, and/or hyaluronic acid-binding domain, if present, can be at any position of the heterodimeric Fc-fused protein. For example, in certain embodiments, where the IL-12 subunits are positioned N-terminal to the antibody Fc domain polypeptides, a proteoglycan-binding domain, a collagen-binding domain, and/or a hyaluronic acid-binding domain as disclosed herein can be fused to the C-terminus of the first antibody Fc domain polypeptide and/or to the C-terminus of the second antibody Fc domain polypeptide. In certain embodiments, where the IL-12 subunits are positioned C-terminal to the antibody Fc domain polypeptides, a proteoglycan-binding domain, a collagen-binding domain, and/or a hyaluronic acid-binding domain as disclosed herein can be fused to the N-terminus of the first antibody Fe domain polypeptide and/or to the N-terminus of the second antibody Fe domain polypeptide.

A proteoglycan-binding domain, collagen-binding domain, and/or hyaluronic acid-binding domain, if present, can be fused to the rest of the heterodimeric Fc-fused protein through a linker. In certain embodiments, the proteoglycan-binding domain is fused to the rest of the heterodimeric Fc-fused protein through a peptide linker. In certain embodiments, the peptide linker includes a spacer peptide disclosed herein.

### Exemplary Heterodimeric Fc-Fused Proteins

In certain embodiments, a heterodimeric Fc-fused protein of the present invention comprises a first polypeptide comprising the amino acid sequence of SEQ ID NO:290 and a second polypeptide comprising the amino acid sequence of SEQ ID NO:291. In certain embodiments, the heterodimeric Fc-fused protein of the present invention comprising SEQ ID NO:290 and SEQ ID NO:291 comprises a Y349C mutation in the CH3 domain of the first antibody Fc domain polypeptide and a S354C mutation in the CH3 domain of the second antibody Fc domain polypeptide. In certain embodiments, the heterodimeric Fc-fused protein of the present invention comprising SEQ ID NO:290 and SEQ ID NO:291 comprise different mutations in the respective Fc domain polypeptide sequences for promoting heterodimerization between the Fc domains.

In certain embodiments, the first polypeptide sequence comprises a first antibody Fc domain polypeptide (human IgG1) sequence comprising K360E and K409W substitutions. In certain embodiments, the second polypeptide sequence comprises a second antibody Fc domain polypeptide (human IgG1) sequence comprising Q347R, D399V, and F405T substitutions. In certain embodiments, the first polypeptide and second polypeptide amino acid sequences comprise one or more mutations for reducing effector functions. In certain embodiments, the heterodimeric Fc-fused protein of the present invention comprises L234A, L235A, and P329A mutations.

In certain embodiments, in the first polypeptide of the heterodimeric Fc-fused protein of the present invention (SEQ ID NO:290), the p40 subunit of human IL-12 is fused to the first antibody Fc domain polypeptide by a first linker comprising a first amino acid sequence, and in the second polypeptide of the heterodimeric Fc-fused protein of the present invention (SEQ ID NO:291), the p35 subunit of human IL-12 is fused to the second antibody Fc domain polypeptide by a second linker comprising a second amino acid sequence.

SEQ ID NO:290 is a sequence of p40 subunit of human IL-12 (underlined amino acids) fused to human IgG1 Fc domain polypeptide. Mutations are shown in bold.

(SEQ ID NO: 290)
IWELKKDVYVVELDWYPDAPGEMVVLTCDTPEEDGITWTLDQSSEVLGSG
KTLTIQVKEFGDAGQYTCHKGGEVLSHSLLLLHKKEDGIWSTDILKDQKE
PKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYEN
YTSSFFIRDIIKPDPPKNLQLKPLKNSRQVEVSWEYPDTWSTPHSYFSLT
FCVQVQGKSKREKKDRVFTDKTSATVICRKNASISVRAQDRYYSSSWSEW
ASVPCSPKSSDKTHTCPPCPAPEAAGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALAAPIEKTISKAKGQPREPQVCTLPPSRDELTE
NQVSLTCLVKGFYPSDIAVEWESNGQPENNYKTTPPVLDSDGSFFLYSWL
TVDKSRWQQGNVFSCSVMHEALHNHYTQKSLSLSPG

SEQ ID NO:291 is a sequence of p35 subunit of human IL-12 (underlined amino acids) fused to human IgG1 Fc domain polypeptide. Mutations are shown in bold.

(SEQ ID NO: 291)
RNLPVATPDPGMFPCLHHSQNLLRAVSNMLQKARQTLEFYPCTSEEIDHE
DITKDKTSTVEACLPLELTKNESCLNSRETSFITNGSCLASRKTSFMMAL
CLSSIYEDLKMYQVEFKTMNAKLLMDPKRQIFLDQNMLAVIDELMQALNF
NSETVPQKSSLEEPDFYKTKIKLCILLHAFRIRAVTIDRVMSYLNASGGG
GSGGGGSGGGGSEPKSSDKTHTCPPCPAPEAAGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALAAPIEKTISKAKGQPREPRVYTLPP
CRDELTKNQVSLTCLVKGFYPSDIAVEWESNGQPENNYKTTPPVLVSDGS
FTLYSKLTVDKSRWQQGNVFSCSVMHEALHNHYTQKSLSLSPG

The first and second polypeptides represented by amino acid sequences SEQ ID NO:290 and SEQ ID NO:291, respectively, form a disulfide bond due to a Y349C mutation in the CH3 domain of the first antibody Fc domain polypeptide sequence (human IgG1) in SEQ ID NO:290 (bolded and underlined) and a S354C mutation in the CH3 domain of the second antibody Fc domain polypeptide sequence (human IgG1) in SEQ ID NO:291 (bolded and underlined), which imparts stability to the heterodimeric Fc-fused protein (Fc numbering according to the EU system).

For promoting heterodimerization between the two Fc domain polypeptides of the heterodimeric Fc-fused protein, the first antibody Fc domain polypeptide sequence (human IgG1) in SEQ ID NO:290 includes K360E and K409W substitutions in the CH3 domain, and the second, different Fc domain polypeptide sequence (human IgG1) in SEQ ID NO:291 includes Q347R, D399V, and F405T substitutions in the CH3 domain (Fc numbering according to the EU system).

The first antibody Fc domain polypeptide sequence and the second, different Fc domain polypeptide sequence (human IgG1) in SEQ ID NO:290 and SEQ ID NO:291 also include L234A, L235A, and P329A (LALAPA) mutations for reducing effector functions.

This heterodimeric Fc-fused protein is herein referred to as DF-hIL-12-Fc si.
(j) Methods of Preparation and Production

The proteins of the present invention can be made using recombinant DNA technology well known to a skilled person in the art. For example, a first nucleic acid sequence encoding a first polypeptide comprising a first subunit of a multisubunit protein sequence fused to a first antibody Fe domain polypeptide can be cloned into a first expression vector; a second nucleic acid sequence encoding a second polypeptide comprising a second, different subunit of a multisubunit protein sequence fused to a second antibody Fc domain polypeptide can be cloned into a second expression vector; and the first and the second expression vectors can be stably transfected together into host cells to produce the multimeric proteins.

To achieve the highest yield of the protein, different ratios of the first and second expression vectors can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of the proteins of the present invention. The proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.
(i) Drug Substance Preparation

In some embodiments, a heterodimeric Fc-fused protein of the present disclosure, e.g. DF hIL12-Fc si, is produced in a eukaryotic cell, e.g., a Chinese Hamster Ovary (CHO) cell. In certain embodiments, a heterodimeric Fc-fused protein of the present disclosure, e.g. DF hIL12-Fc si, is produced in a CHO cell in suspension culture (e.g., in a shake flask). In certain embodiments, a vial of CHO cells is thawed and passaged more than one time before the protein is produced (e.g., twice, thrice, four times, five times, 6 times). In certain embodiments, a vial of CHO cells is thawed and passaged four times before the protein is produced. In certain embodiments, CHO cells from the fourth passage are used to inoculate a culture in a first bioreactor. In certain embodiments, the first bioreactor has a volume of about 40 L, about 45 L, about 50 L, about 55 L, or about 60 L. In certain embodiments, the first bioreactor has a volume of about 50 L. In certain embodiments, the CHO cells from the culture of the first bioreactor are used to inoculate a culture in a production bioreactor. In certain embodiments, the production bioreactor has a volume of about 180 L, about 185 L, about 190 L, about 195 L, about 200 L, about 205 L, about 210 L, about 215 L, or about 220 L. In certain embodiments, the final culture volume in the production bioreactor is about 180 L. In certain embodiments, the CHO cells are grown in growth media supplemented with L-glutamine (e.g., 6 mM L-glutamine). In certain embodiments, the CHO cells are grown at a temperature of about 37° C. In certain embodiments, culture conditions are monitored daily (e.g., for glucose, for lactate, for pH).

In some embodiments, the production bioreactor regulates dissolved oxygen in the culture with air and oxygen supplementation. In some embodiments, the production bioreactor regulates pH with addition of carbon dioxide gas and/or sodium carbonate base. In some embodiments, the production bioreactor is sampled daily for cell density and viability until a target cell viability is met. In certain embodiments, the target cell viability is about 10×10⁶ viable cells/mL, about 11×10⁶ viable cells/mL, about 12×10⁶ viable cells/mL, about 13×10⁶ viable cells/mL, about 14×10⁶ viable cells/mL, about 15×10⁶ viable cells/mL, about 16×10⁶ viable cells/mL, about 17×10⁶ viable cells/mL, about 18×10⁶ viable cells/mL, about 19×10⁶ viable cells/mL, or about 20×10⁶ viable cells/mL. In certain embodiments, the target cell viability is greater than about 14×10⁶ viable cells/mL. In certain embodiments, after target cell viability is met, the temperature is shifted from about 37° C. to about 33° C. for culture harvest. In certain embodiments, the culture is harvested when the viability is higher than about 80%, about 81%, about 82%, about 83%, about 84%, about 85%, about 86%, about 87%, about 88%, about 89%, about 90%. In certain embodiments, the culture is harvested when the viability is higher than about 85%. In certain embodiments, culture conditions are monitored daily during the culture period (e.g., for glucose, for lactate, for pH). In certain embodiments, titer of DF hIL12-Fc si is monitored in the culture, starting at about Day 8 (e.g., Day 6, Day 7, Day 8, Day 9, or Day 10). In certain embodiments, the culture is supplemented with concentrated nutrient feeds, concentrated glucose solution, and/or antifoam. In some embodiments, the cells are cultured for about 7-about 21 days, about 8-about 20 days, about 9-about 19 days, about 10-about 18 days, about 11-about 17 days, about 12-about 16 days, or about 11-about 15 days. In certain embodiments, the cells are cultured for about 14 days.

In some embodiments, the production bioreactor is clarified by depth filtration prior to purification of a protein of the present disclosure, e.g., DF hIL12-Fc si. In certain embodiments, a two-stage single-use depth filtration system consisting of DOHC and XOHC filters is used for the clarification. In certain embodiments, before filtration, the production bioreactor temperature is adjusted to about 18° C. and the dissolved oxygen setpoint is increased to about 70% of saturation. In certain embodiments, the harvest filters are rinsed with water for injection (WFI) and then equilibrated with buffer. In some embodiments, the cell suspension is passed through the harvest filters using a peristaltic pump and the filters are flushed to collect the product. In certain embodiments, pressure is monitored and maintained at about less than 25 psig (e.g., about less than 25 psig, about less than 20 psig, or about less than 15 psig). The filtrate is then filtered through a 0.45/0.2 μm membrane into storage, e.g., in a sterile bag.

In some embodiments, purification of a heterodimeric Fc-fused protein of the present disclosure, e.g. DF hIL12-Fc si, comprises or consists of three chromatography steps and two virus clearance steps. In certain embodiments, the three chromatography steps comprise or consist of Protein A Capture Chromatography, Mixed Mode Chromatography, and Cation Exchange Chromatography. In certain embodiments, clarified harvest comprising the heterodimeric Fc-fused protein of the present disclosure, e.g. DF hIL12-Fc si, is captured by Protein A capture chromatography (e.g., using a Protein A resin column). In certain embodiments, the Protein A capture chromatography removes process-related impurities (e.g., DNA, host cell proteins), media additives, and allows for volume reduction. In certain embodiments, the Protein A resin column is first equilibrated with a buffer comprising 20 mM Tris, 150 mM NaCl, at a pH of about 7.5. In certain embodiments, after loading, the column is washed with equilibration buffer to remove unbound or loosely bound impurities, In certain embodiments, after the first wash, the column is washed a second time with a buffer comprising 50 mM acetate at a pH of about 5.4. In certain embodiments, the second wash lowers the pH and prepares the column for elution. In certain embodiments, DF hIL12-Fc si is eluted with a buffer comprising 50 mM acetate, 100 mM arginine at a pH of about 3.7. In certain embodiments, DF hIL12-Fc si is collected by 280 nm UV wavelength starting at 1.25 AU/cm ascending and then ending at 1.25 AU/cm descending. In certain embodiments, the eluate is collected in one pool, and each column cycle is individually processed by low pH virus inactivation.

In certain embodiments, the virus clearance steps comprise low pH inactivation and nanofiltration. In certain embodiments, the protein A eluate is incubated at low pH to inactive potentially present viruses. In certain embodiments, pH of the eluate is adjusted with acetic acid, e.g., 0.5 M acetic acid, and incubated for at least 30 minutes, at least 35 minutes, at least 40 minutes, at least 45 minutes, at least 50 minutes, at least 55 minutes, at least 60 minutes, at least 65 minutes, at least 70 minutes, at least 75 minutes, at least 80 minutes, at least 85 minutes, or at least 90 minutes. In certain embodiments, pH of the eluate is adjusted with acetic acid, e.g., 0.5 M acetic acid, and incubated for at least 60 minutes. In certain embodiments, the acetic acid adjusts the pH to about 3.55 to 3.75, e.g., about 3.60 to 3.70, or about 3.65. In certain embodiments, the acetic acid adjusts the pH to about 3.65. In certain embodiments, after low pH incubation, pH is raised, e.g., with Tris base, e.g., with 2 M Tris base. In certain embodiments, pH is raised to a neutralization pH of about 5.1, about 5.2, or about 5.3. In certain embodiments, the protein A eluate is filtered through a 0.2 μm filtration assembly. In certain embodiments, the low pH inactivation precedes the nanofiltration. In certain embodiments, the nanofiltration precedes the low pH inactivation.

In some embodiments, after the virus clearance steps, the pool is filtered through an intermediate depth filter, e.g., X0SP intermediate depth filter. In certain embodiments, DF hIL12-Fc si is loaded at a range of about 500-about 1000 g/m² (e.g., about 400-about 1100 g/m², about 450-about 1050 g/m², about 500-about 1000 g/m²). In certain embodiments, the X0SP pool conductivity is adjusted to less than 6.0 mS/cm with acetate, e.g. 50 mM acetate pH of about 5.2, prior to Mixed Mode Chromatography.

In some embodiments, Mixed Mode Chromatography is performed to remove high molecular weight (HMW) species. In certain embodiments, the column is equilibrated, e.g., with a buffer comprising 50 mM Acetate at pH of about 5.2, and loaded. In certain embodiments, after loading, the column is washed, e.g., with a buffer comprising 50 mM Acetate and 250 mM NaCl at pH of about 5.2. In certain embodiments, collection is initiated by 280 nm UV detection at 0.625 AU/cm ascending and ended at 1.50 AU/cm descending. In certain embodiments, after collection, each cycle is passed through a filter train containing a terminal 0.2 μm filter.

In some embodiments, Cation Exchange Chromatography is performed to remove product-related impurities (e.g., HMW species, low molecular weight (LMW) species) and process-related impurities. In some embodiments, multiples cycles of Cation Exchange Chromatography are performed (e.g., 1, 2, 3, 4, 5, 6, 7, 8, 9, or 10 or more cycles) for each product lot. In certain embodiments, the cycles are pooled and diluted, e.g., with a buffer comprising 50 mM Tris at pH 7.4. In certain embodiments, the pooled samples are adjusted to pH of about 7.3, about 7.4, about 7.5, about 7.6, or about 7.7 with a base solution, e.g., Tris, e.g., 2 M Tris base. In certain embodiments, the pooled samples are adjusted to pH of about 7.5. In certain embodiments, the column is equilibrated with a buffer comprising 50 mM Tris at pH 7.4. In certain embodiments, elution comprises a gradient of 50 mM Tris at pH of about 7.4 (Buffer A) and 50 mM Tris and 0.5 M NaCl at pH of about 7.4 (Buffer B). In certain embodiments, product collection is initiated by 280 nm UV detection starting at 2.5 AU/cm ascending and ending at 4.5 AU/cm descending. In certain embodiments, after collection, each cycle is passed through a filter train containing a terminal 0.2 μm filter.

In some embodiments, nanofiltration of the cycles from the Cation Exchange Chromatography removes viruses. In certain embodiments, the eluate first passes through a prefilter and a nominal filter (e.g., a nominal filter of about 20 nm). In certain embodiments, the system is equilibrated with a buffer, e.g., a buffer comprising 50 mM Tris and 265 mM NaCl at pH of about 7.4. In certain embodiments, after loading, the system is rinsed with equilibration buffer, e.g., a buffer comprising 50 mM Tris and 265 mM NaCl at of about pH 7.4. In certain embodiments, the filtrate is filtered through a membrane, e.g., a 0.2 μm membrane.

In some embodiments, the filtrate undergoes ultrafiltration and diafiltration (UF/DF). In certain embodiments, ultrafiltration and diafiltration are performed using a molecular weight cut-off membrane of about 20 kDa, about 25 kDa, about 30 kDa, about 35 kDa, or about 40 kDa. In certain embodiments, ultrafiltration and diafiltration are performed using a molecular weight cut-off membrane of about 30 kDa. In certain embodiments, the system is equilibrated with a buffer, e.g., a buffer comprising 50 mM Tris and 265 mM NaCl at pH of about 7.4. In certain embodiments, the viral filtrate pool is concentrated to a target of about 5.0 g/L. In certain embodiments, buffer exchange is performed against at least 7 diavolumes (e.g., 7, 8, or 9 diavolumes) of buffer comprising 20 mM Citrate at pH of about 6.5. In some embodiments, after diafiltration, a second concentration step is performed to target about 11.0 g/L. In certain embodiments, the product is diluted to a final target concentration of about 7.5 g/L in diafiltration buffer.

In some embodiments, a 20 mM Citrate, 18% (w/v) Sucrose, 3% (w/v) Mannitol, 0.03% (w/v) polysorbate-80, pH 6.5 stock solution is spiked into the UF/DF pool to target a final concentration of 20 mM Citrate, 6% (w/v) Sucrose, 1% (w/v) Mannitol, 0.01% (w/v) polysorbate-80 in the drug substance.

In certain embodiments, the formulated retentate is filtered, e.g., through a 0.2 μm membrane, into the final drug substance storage containers. In certain embodiments, the final fill volume is about 1.0 L. In certain embodiments, the final substance storage containers comprise 2 L polycarbonate bottles with polypropylene closures. In certain embodiments, each bottle is aseptically sampled, labeled, and frozen at less than −65° C. (e.g., −65° C., −70° C., −75° C., −80° C., or lower).
(ii) Drug Product Preparation

In some embodiments, frozen drug substance comprising a heterodimeric Fc-fused protein of the present disclosure, e.g. DF hIL12-Fc si, is thawed for more than 96 hours (e.g., 96 hours, 120 hours, 144 hours, 168 hours, or more), at about 2-8° C., in the dark. In certain embodiments, a buffer consisting of 20 mM citrate, 6% (w/v) sucrose, 1% (w/v) mannitol, 0.01% polysorbate 80 (w/v) at pH 6.0 is prepared. In certain embodiments, the buffer is prepared by adding solid sodium citrate dihydrate, citric acid monohydrate, sucrose, and mannitol to water for injection (WFI) and mixing until dissolution. In certain embodiments, citrate in the drug product comprises or consists of solid sodium citrate dihydrate and/or citric acid monohydrate. In some embodiments, a polysorbate 80 stock solution is prepared in WFI and added to the buffer. In certain embodiments, acceptance pH of the buffer is about 6.5±0.4 (e.g., pH 6.1, pH 6.2, pH 6.3, pH 6.4, pH 6.5, pH 6.6, pH 6.7, pH 6.8, or pH 6.9). In certain embodiments, the buffer is diluted with WFI and tested for acceptance pH of about 6.5±0.4 (e.g., pH 6.1, pH 6.2, pH 6.3, pH 6.4, pH 6.5, pH 6.6, pH 6.7, pH 6.8, or pH 6.9) and osmolality. In certain embodiments, the buffer is filtered through a membrane, e.g., a 0.2 μm membrane.

In some embodiments, the weight of drug substance is used to calculate a target batch volume. In certain embodiments, the drug substance is added to the buffer in a carboy (e.g., a 10 L carboy) to approximately 80% of the calculated batch volume and mixed. In certain embodiments, the 80% drug product solution is tested for acceptance pH acceptance of about 6.5±0.3 (e.g., pH 6.2, pH 6.3, pH 6.4, pH 6.5, pH 6.6, pH 6.7, or pH 6.8). In certain embodiments, the 80% drug product solution is tested for protein concentration by absorbance at 280 nm using an Extinction coefficient of 1.44 L/(g*cm).

In some embodiments, the buffer components are designed to yield a pH of about 6.5. In certain embodiments, at the buffer steps, a titration with 1N sodium hydroxide or 1N hydrochloric acid may be performed to bring the pH within the acceptance pH of about 6.5±0.4 (e.g., pH 6.1, pH 6.2, pH 6.3, pH 6.4, pH 6.5, pH 6.6, pH 6.7, pH 6.8, or pH 6.9). In certain embodiments, at the 80% bulk drug product steps, a titration with 1N sodium hydroxide or 1N hydrochloric acid may be performed to bring the pH within the acceptance pH of about 6.5±0.3 (e.g., pH 6.2, pH 6.3, pH 6.4, pH 6.5, pH 6.6, pH 6.7, or pH 6.8).

In some embodiments, the target concentration of a heterodimeric Fc-fused protein of the present disclosure, e.g. DF hIL12-Fc si, is about 1 mg/mL. In some embodiments, the protein concentration is verified by absorbance at 280 nm with acceptance criteria 1.0±0.2 mg/mL (e.g., 0.8 mg/mL, 0.9 mg/mL, 1.0 mg/mL, 1.1 mg/mL, 1.2 mg/mL). In some embodiments, samples are taken to confirm the acceptance pH of about 6.5±0.3 (e.g., pH 6.2, pH 6.3, pH 6.4, pH 6.5, pH 6.6, pH 6.7, or pH 6.8) and osmolality.

In some embodiments, the compounded bulk drug product solution is passed through a filter, e.g., a sterile 0.2 μm filter, into a carboy, e.g., a 10 L carboy, for bioburden reduction, and held until sterile filtration and filling.

In some embodiments, the bulk drug product is filtered through two filter capsules in series, each filter capsule consisting of a 0.45 μm polyethersulfone (PES) pre-filter membrane and a 0.2 μm PES sterilizing membrane. In certain embodiments, the drug product is filtered into a sterile, disposable fill bag inside a controlled Grade B area of the filling suite. In certain embodiments, both sterilizing filter capsules are tested for integrity by bubble point after filtration, with acceptance criteria greater than 3200 mbar, using WFI.

In some embodiments, the bulk drug product solution is filled from the disposable bag residing immediately outside of the restricted access barrier system (RABS). In certain embodiments, the product is filled into vials, e.g., ready-to-use 2R borosilicate type I vials.

In certain embodiments, the vials are stoppered, e.g., with sterilized, 13 mm serum stoppers and capped with 13 mm aluminum overseals. In certain embodiments, the fill volume of the vial is 1.3 mL±5% (i.e., from 1.235 mL to 1.365 mL). In certain embodiments, vials are moved to 2-8° C. storage. In certain embodiments, vials are stored at 2° C., 3° C., 4° C., 5° C., 6° C., 7° C., or 8° C.
(k) Pharmaceutical Formulation

The present disclosure also features pharmaceutical compositions that contain an effective amount of a protein described herein. The composition can be formulated for use in a variety of drug delivery systems. One or more physiologically acceptable excipient(s) or carrier(s) can also be included in the composition for proper formulation. The term “excipient,” as used herein, means any non-therapeutic agent added to the formulation to provide a desired physical or chemical property, for example, pH, osmolarity, viscosity, clarity, color, isotonicity, odor, sterility, stability, rate of dissolution or release, adsorption, or penetration.
(i) Excipients and pH

The one or more excipients in the pharmaceutical formulation of the present invention comprises a buffering agent. The term “buffering agent,” as used herein, refers to one or more components that when added to an aqueous solution is able to protect the solution against variations in pH when adding acid or alkali, or upon dilution with a solvent. In addition to phosphate buffers, glycinate, carbonate, citrate, histidine buffers and the like can be used, in which case, sodium, potassium or ammonium ions can serve as counterion.

In certain embodiments, the buffer or buffer system comprises at least one buffer that has a buffering range that overlaps fully or in part with the range of pH 5.5-7.4. In certain embodiments, the buffer has a pKa of about 6.5±0.5. In certain embodiments, the buffer comprises a citrate buffer. In specific embodiments, the citrate buffer comprises sodium citrate dihydrate and citric acid monohydrate. In certain embodiments, the citrate is present at a concentration of about 5 to about 100 mM, about 10 to about 100 mM, about 15 to about 100 mM, about 20 to about 100 mM, about 5 to about 50 mM, about 10 to about 50 mM, about 15 to about 100 mM, about 20 to about 100 mM, about 5 to about 25 mM, about 10 to about 25 mM, about 15 to about 25 mM, about 20 to about 25 mM, about 5 to about 20 mM, about 10 to about 20 mM, or about 15 to about 20 mM. In certain embodiments, the citrate is present at a concentration of about 5 mM, about 10 mM, about 15 mM, about 20 mM, about 25 mM, or about 50 mM. In certain embodiments, the citrate is present at a concentration of 20 mM.

The pharmaceutical formulation of the present invention may have a pH of 6.0 to 7.0. For example, in certain embodiments, the pharmaceutical formulation has a pH of 6.0 to 7.0 (i.e., 6.5±0.5), 6.1 to 6.9 (i.e., 6.5±0.4), 6.2 to 6.8 (i.e., 6.5±0.3), 6.3 to 6.7 (i.e., 6.5±0.2), 6.4 to 6.6 (i.e., 6.5±0.1), or 6.45 to 6.65 (i.e., 6.5±0.05). In certain embodiments, the pharmaceutical formulation has a pH of about 6.0, about 6.1, about 6.2, about 6.3, about 6.4, about 6.5, about 6.6, about 6.7, about 6.8, about 6.9, or about 7.0. In certain embodiments, the pharmaceutical formulation has a pH of 6.5. Under the rules of scientific rounding, a pH greater than or equal to 6.45 and smaller than or equal to 6.55 is rounded as 6.0.

In certain embodiments, the buffer system of the pharmaceutical formulation comprises citrate at 10 to 25 mM, at a pH of 6.5±0.2. In certain embodiments, the buffer system of the pharmaceutical formulation comprises citrate at 20 mM, at a pH of 6.5±0.2. In certain embodiments, the buffer system of the pharmaceutical formulation comprises citrate at 10 to 25 mM, at a pH of 6.5±0.05. In certain embodiments, the buffer system of the pharmaceutical formulation comprises citrate at 20 mM, at a pH of 6.5±0.05.

The one or more excipients in the pharmaceutical formulation of the present invention further comprises a sugar or sugar alcohol. Sugars and sugar alcohols are useful in pharmaceutical formulations as a thermal stabilizer. In certain embodiments, the pharmaceutical formulation comprises a sugar, for example, a monosaccharide (e.g., glucose, xylose, or erythritol), a disaccharide (e.g., sucrose, trehalose, maltose, or galactose), or an oligosaccharide (e.g., stachyose). In specific embodiments, the pharmaceutical formulation comprises sucrose. In certain embodiments, the pharmaceutical formulation comprises a sugar alcohol, for example, a sugar alcohol derived from a monosaccharide (e.g., mannitol, sorbitol, or xylitol), a sugar alcohol derived from a disaccharide (e.g., lactitol or maltitol), or a sugar alcohol derived from an oligosaccharide. In specific embodiments, the pharmaceutical formulation comprises mannitol.

The amount of the sugar or sugar alcohol contained within the formulation can vary depending on the specific circumstances and intended purposes for which the formulation is used. In certain embodiments, the pharmaceutical formulation comprises 0% w/v-about 12% w/v, about 1% w/v-about 11% w/v, about 2% w/v-about 10% w/v, about 30% w/v-about 9% w/v, about 3% w/v-about 12% w/v, about 4% w/v-about 8% w/v, or about 5% w/v-about 7% w/v of the sugar or sugar alcohol. In certain embodiments, the pharmaceutical formulation comprises 0% w/v-about 2% w/v, about 0.5% w/v-about 1.5% w/v, about 0.6% w/v-about 1.4% w/v, about 0.7% w/v-about 1.3% w/v, about 0.8% w/v-about 1.2% w/v, or about 0.9% w/v-about 1.1% w/v of the sugar or sugar alcohol. In certain embodiments, the pharmaceutical formulation comprises about 0% w/v, about 0.5% w/v, about 1% w/v, about 2% w/v, about 3% w/v, about 4% w/v, about 5% w/v, about 6% w/v, about 7% w/v, about 8% w/v, about 9% w/v, or about 10% w/v of the sugar or sugar alcohol. In specific embodiments, the pharmaceutical formulation comprises about 6% w/v of the sugar or sugar alcohol (e.g., sucrose). In specific embodiments, the pharmaceutical formulation comprises about 1% w/v of the sugar or sugar alcohol (e.g., mannitol). In specific embodiments, the pharmaceutical formulation comprises about 6% w/v of the sugar or sugar alcohol (e.g., sucrose) and about 1% w/v of a second sugar or sugar alcohol (e.g., mannitol).

The one or more excipients in the pharmaceutical formulation disclosed herein further comprises a surfactant. The term “surfactant,” as used herein, refers to a surface active molecule containing both a hydrophobic portion (e.g., alkyl chain) and a hydrophilic portion (e.g., carboxyl and carboxylate groups). Surfactants are useful in pharmaceutical formulations for reducing aggregation of a therapeutic protein. Surfactants suitable for use in the pharmaceutical formulations are generally non-ionic surfactants and include, but are not limited to, polysorbates (e.g., polysorbates 20 or 80); poloxamers (e.g., poloxamer 188); sorbitan esters and derivatives; Triton; sodium laurel sulfate; sodium octyl glycoside; lauryl-, myristyl-, linoleyl-, or stearyl-sulfobetadine; lauryl-, myristyl-, linoleyl- or stearyl-sarcosine; linoleyl-, myristyl-, or cetyl-betaine; lauramidopropyl-cocamidopropyl-, linoleamidopropyl-, myristamidopropyl-, palmidopropyl-, or isostearamidopropylbetaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethylene glycol, polypropyl glycol, and copolymers of ethylene and propylene glycol (e.g., Pluronics, PF68 etc.). In certain embodiments, the surfactant is a polysorbate. In certain embodiments, the surfactant is polysorbate 80.

The amount of a non-ionic surfactant contained within the pharmaceutical formulation of the present invention may vary depending on the specific properties desired of the formulation, as well as the particular circumstances and purposes for which the formulations are intended to be used. In certain embodiments, the pharmaceutical formulation comprises 0.005% to about 0.5%, about 0.005% to about 0.2%, about 0.005% to about 0.1%, about 0.005% to about 0.05%, about 0.005% to about 0.02%, about 0.005% to about 0.01%, about 0.01% to about 0.5%, about 0.01% to about 0.2%, about 0.01% to about 0.1%, about 0.01% to about 0.05%, or about 0.01% to about 0.02% of the non-ionic surfactant (e.g., polysorbate 80). In certain embodiments, the pharmaceutical formulation comprises about 0.005%, about 0.01%, about 0.02%, about 0.03%, about 0.04%, about 0.05%, about 0.06%, about 0.07%, about 0.08%, about 0.09%, about 0.1%, about 0.15%, about 0.2%, about 0.25%, about 0.3%, about 0.35%, about 0.4%, about 0.45%, or about 0.5% of the non-ionic surfactant (e.g., polysorbate 80).

The pharmaceutical formulation of the present invention may further comprise one or more other substances, such as a bulking agent or a preservative. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents. A preservative reduces bacterial action and may, for example, facilitate the production of a multi-use (multiple-dose) formulation.
(ii) Exemplary Formulations

In certain embodiments, the pharmaceutical formulation of the present invention comprises the heterodimeric Fc-fused protein, citrate, a sugar (e.g., sucrose), a sugar alcohol (e.g., mannitol), and a polysorbate (e.g., polysorbate 80), at pH 6.0 to 7.0.

In certain embodiments, the pharmaceutical formulation comprises 0.5 to 1.5 mg/mL of the heterodimeric Fc-fused protein, 10 to 30 mM of citrate, 4% w/v to 8% w/v of a sugar (e.g., sucrose), 0.5% w/v to 1.5% w/v of a sugar alcohol (e.g., mannitol), and 0.005% to 0.05% of a polysorbate (e.g., polysorbate 80), at pH 6.5 to 7.5. In certain embodiments, the pharmaceutical formulation comprises 0.5 to 1.5 mg/mL of the heterodimeric Fc-fused protein, 20 mM of citrate, 6% w/v of a sugar (e.g., sucrose), 0.5% w/v to 1.5% w/v of a sugar alcohol (e.g., mannitol), and 0.01% of a polysorbate (e.g., polysorbate 80), at pH 6.0 to 7.0. In certain embodiments, the pharmaceutical formulation comprises 0.5 to 1.5 mg/mL of the heterodimeric Fc-fused protein, 20 mM of citrate, 6% w/v of a sugar (e.g., sucrose), 1% w/v of a sugar alcohol (e.g., mannitol), and 0.01% of a polysorbate (e.g., polysorbate 80), at pH 6.3 to 6.7. In certain embodiments, the pharmaceutical formulation comprises 0.5 to 1.5 mg/mL of the heterodimeric Fc-fused protein, 20 mM of citrate, 6% w/v of a sugar (e.g., sucrose), 1% w/v of a sugar alcohol (e.g., mannitol), and 0.01% of a polysorbate (e.g., polysorbate 80), at pH 6.45 to 6.55.
(iii) Stability of the Heterodimeric Fc-fused Protein and Formulation

The pharmaceutical formulations of the present invention exhibit high levels of stability. A pharmaceutical formulation is stable when the heterodimeric Fc-fused protein within the formulation retains an acceptable degree of physical property, chemical structure, and/or biological function after storage under defined conditions. In certain embodiments, the pharmaceutical formulation is a clear liquid, free of visible particulates. In certain embodiments, the thermal stability is tested at 5° C., 50° C., and following freeze-thaw cycles (e.g., 5 freeze-thaw cycles).

Stability can be measured by determining the percentage of the heterodimeric Fc-fused protein in the formulation that remains in a native conformation after storage for a defined amount of time at a defined temperature. The percentage of a protein in a native conformation can be determined by, for example, size exclusion chromatography (e.g., size exclusion high performance liquid chromatography, SEC-HPLC), where a protein in the native conformation is not aggregated (eluted in a high molecular weight fraction) or degraded (eluted in a low molecular weight fraction). In certain embodiments, more than about 95%, more than about 96%, more than about 97%, more than about 98%, or more than about 99% of the heterodimeric Fc-fused protein has native conformation, as determined by size-exclusion chromatography, after incubation at 2-8° C. for 2 weeks. In certain embodiments, more than about 95%, more than about 96%, more than about 97%, more than about 98%, or more than about 99% of the heterodimeric Fc-fused protein has native conformation, as determined by size-exclusion chromatography, after freeze-thaw. In certain embodiments, more than about 75%, more than about 76%, more than about 77%, more than about 78%, more than about 79%, more than about 80%, more than about 81%, more than about 82%, more than about 83%, more than about 84%, or more than about 85% of the heterodimeric Fc-fused protein has native conformation, as determined by size-exclusion chromatography, after incubation at 50° C. for 2 weeks. In certain embodiments, less than about 0.5%, less than about 0.6%, less than about 0.7%, less than about 0.8%, less than about 0.9%, or less than about 1% of the heterodimeric Fc-fused protein forms a high molecular weight complex (i.e., having a higher molecular weight than the native protein), as determined by size-exclusion chromatography, after incubation at 2-8° C. for 2 weeks. In certain embodiments, less than about 40%, less than about 30%, less than about 20%, less than about 10%, less than about 5%, less than about 4%, less than about 3%, less than about 2%, or less than about 1% of the heterodimeric Fc-fused protein form a high molecular weight complex (i.e., having a higher molecular weight than the native protein), as determined by size-exclusion chromatography, after incubation at 50° C. for 2 weeks. In certain embodiments, less than about 0.5%, less than about 0.6%, less than about 0.7%, less than about 0.8%, less than about 0.9%, or less than about 1% of the heterodimeric Fc-fused protein forms a high molecular weight complex (i.e., having a higher molecular weight than the native protein), as determined by size-exclusion chromatography, after freeze-thaw. In certain embodiments, less than about 0.1%, less than about 0.2%, less than about 0.3%, less than about 0.4%, less than about 0.5%, less than about 0.6%, less than about 0.7%, less than about 0.8%, less than about 0.9%, or less than about 1% of the heterodimeric Fc-fused protein is degraded (i.e., having a lower molecular weight than the native protein), as determined by size-exclusion chromatography, after incubation at 2-8° C. for 2 weeks. In certain embodiments, less than about 1%, less than about 1.5%, less than about 2%, less than about 2.5%, or less than about 3% of the heterodimeric Fc-fused protein is degraded (i.e., having a lower molecular weight than the native protein), as determined by size-exclusion chromatography, after incubation at 50° C. for 2 weeks. In certain embodiments, less than about 0.1%, less than about 0.2%, less than about 0.3%, less than about 0.4%, less than about 0.5%, less than about 0.6%, less than about 0.7%, less than about 0.8%, less than about 0.9%, or less than about 1% of the heterodimeric Fc-fused protein is degraded (i.e., having a lower molecular weight than the native protein), as determined by size-exclusion chromatography, after freeze-thaw.

SEC-HPLC can provide a measure of the purity of a pharmaceutical formulation by the percentage of protein, e.g., the heterodimeric Fc-fused protein, in the main peak. A purity profile is determined by the area of the main peak as a percentage of total detected area in a SEC-HPLC analysis. In some embodiments, the purity profile of the pharmaceutical formulation, is greater than about 90%, greater than about 91%, greater than about 92%, greater than about 93%, greater than about 94%, greater than about 95%, greater than about 96%, greater than about 97%, greater than about 98%, or greater than about 99%. In certain embodiments, the purity profile of the pharmaceutical formulation, is about 99.0%. In some embodiments, the purity profile of the pharmaceutical formulation, is greater than about 75%, greater than about 80%, greater than about 81%, greater than about 82%, greater than about 83%, greater than about 84%, or greater than about 85%, after the pharmaceutical formulation is incubated for 2 weeks at 50° C. In certain embodiments, the purity profile of the pharmaceutical formulation, is about 85.2%. In some embodiments, the purity profile of the pharmaceutical formulation, is greater than about 90%, greater than about 91%, greater than about 92%, greater than about 93%, greater than about 94%, greater than about 95%, greater than about 96%, greater than about 97%, greater than about 98%, or greater than about 98.5% after the pharmaceutical formulation is subjected to five freeze thaw cycles. In certain embodiments, the purity profile of the pharmaceutical formulation, is about 98.9%.

Stability can also be measured by determining the parameters of a protein solution by dynamic light scattering. The Z-average and polydispersity index (PDI) values indicate the average diameter of particles in a solution and these measures increase when aggregates are present in the solution. The monomer % Pd value indicates the spread of different monomers detected, where lower values indicate a monodisperse solution, which is preferred. The monomer size detected by DLS is useful in confirming that the main population is monomer and to characterize any higher order aggregates that may be present. In certain embodiments, the Z-average value of the pharmaceutical formulation does not increase by more than 5%, 10%, or 15% after incubation at 2-8° C. for 2 weeks. In certain embodiments, the Z-average value of the pharmaceutical formulation does not increase by more than 2-fold, 3-fold, 4-fold, or 5-fold after freeze-thaw. In certain embodiments, the Z-average value of the pharmaceutical formulation does not increase by more than about 10%, more than about 20%, more than about 30%, more than about 40%, more than about 50%, more than about 60%, more than about 70%, more than about 80%, more than about 90%, more than about 100%, more than about 150%, or more than about²⁰⁰% after incubation at 50° C. for 2 weeks. In some embodiments, the heterodimeric Fc-fused protein in the pharmaceutical formulation has a Z-average hydrodynamic diameter of less than about 15 nm, less than about 14 nm, less than about 13 nm, or less than about 12 nm, as measured by dynamic light scattering at 25° C. In specific embodiments, the heterodimeric Fc-fused protein in the pharmaceutical formulation has a Z-average hydrodynamic diameter of less than about 11.6 nm as measured by dynamic light scattering at 25° C. In some embodiments, the heterodimeric Fc-fused protein in the pharmaceutical formulation has a Z-average hydrodynamic diameter of less than about 20 nm, less than about 19 nm, less than about 18 nm, less than about 17 nm, less than about 16 nm, less than about 15.5, less than about 15 nm, or less than about 14.5, as measured by dynamic light scattering at 25° C., after the pharmaceutical formulation is incubated for 2 weeks at 50° C. In specific embodiments, the heterodimeric Fc-fused protein in the pharmaceutical formulation has a Z-average hydrodynamic diameter of about 14.4 nm. In some embodiments, the heterodimeric Fc-fused protein in the pharmaceutical formulation has a Z-average hydrodynamic diameter of less than about 20 nm, less than about 19 nm, less than about 18 nm, less than about 17 nm, less than about 16.5 nm, less than about 16 nm, or less than about 15.5 nm as measured by dynamic light scattering at 25° C., after the pharmaceutical formulation is subjected to five freeze thaw cycles. In certain embodiments, the heterodimeric Fc-fused protein in the pharmaceutical formulation has a Z-average hydrodynamic diameter of about 15.3 nm.

In certain embodiments, the PDI value of the pharmaceutical formulation does not increase by more than about 2-fold, about 3-fold, about 4-fold, or about 5-fold after incubation at 2-8° C. for 2 weeks. In certain embodiments, the PDI value of the pharmaceutical formulation does not increase by more than about 2-fold, about 3-fold, about 4-fold, about 5-fold, or about 6-fold after freeze-thaw. In certain embodiments, the PDI value of the pharmaceutical formulation does not increase by more than about 2-fold, about 3-fold, about 4-fold, about 5-fold, or about 6-fold after incubation at 50° C. for 2 weeks. In some embodiments, the polydispersity index of the heterodimeric Fc-fused protein in the pharmaceutical formulation is less than about 0.30, less than about 0.29, less than about 0.28, less than about 0.27, less than about 0.26, or less than about 0.25 as measured by dynamic light scattering at 25° C. In certain embodiments, the polydispersity index of the heterodimeric Fc-fused protein in the pharmaceutical formulation is about 0.26. In some embodiments, the polydispersity index of the heterodimeric Fc-fused protein in the pharmaceutical formulation is less than about 0.30, less than about 0.29, less than about 0.28, less than about 0.27, or less than about 0.26 as measured by dynamic light scattering at 25° C., after the pharmaceutical formulation is incubated for 2 weeks at 50° C. In certain embodiments, the polydispersity index of the heterodimeric Fc-fused protein in the pharmaceutical formulation is about 0.25. In some embodiments, the polydispersity index of the heterodimeric Fc-fused protein in the pharmaceutical formulation is less than about 0.40, less than about 0.35, or less than about 0.34, as measured by dynamic light scattering at 25° C., after the pharmaceutical formulation is subjected to five freeze thaw cycles. In certain embodiments, the polydispersity index of the heterodimeric Fc-fused protein in the pharmaceutical formulation is about 0.33.

Stability can also be measured by determining the thermal stability of a protein solution by differential scanning fluorimetry (DSF). DSF allows for quantification of changes in the thermal denaturation temperature and stability of a protein under varying test conditions, e.g., buffer or pH. In some embodiments, DSF provides two thermal unfolding temperatures (also known as melting temperatures), Tₘ₁ and Tₘ₂. In certain embodiments, the Tₘ₁ of the pharmaceutical formulation is greater than about 60° C., greater than about 61° C., greater than about 62° C., greater than about 63° C., greater than about 64° C., greater than about 65° C., or greater than about 66° C. In certain embodiments, the Tₘ₁ of the pharmaceutical formulation is greater than about 70° C., greater than about 71° C., greater than about 72° C., greater than about 73° C., greater than about 74° C., greater than about 75° C., greater than about 76° C., or greater than about 77° C. In specific embodiments, the pharmaceutical formulation has a thermal stability profile defined by a Tₘ₁ of about 67.0° C. and a Tₘ₂ of about 77.3° C. In certain embodiments, the Tₘ₁ and/or Tₘ₂ is changed by less than 2° C., less than 1.5° C., or less than 1° C. when the pharmaceutical formulation is incubated for 1 week at 50° C., as compared to the same pharmaceutical formulation that is incubated for 1 week at 5° C. In specific embodiments, the Tₘ₁ is changed by less than 1° C. when the pharmaceutical formulation is incubated for 1 week at 50° C., as compared to the same pharmaceutical formulation that is incubated for 1 week at 5° C. In specific embodiments, the Tₘ₂ is changed by less than 1° C. when the pharmaceutical formulation is incubated for 1 week at 50° C., as compared to the same pharmaceutical formulation that is incubated for 1 week at 5° C.

In some embodiments, DSF provides the temperature at which protein aggregation begins to occur, T_{agg}. In some embodiments, the T_{agg}. of the pharmaceutical formulation is greater than 60° C., greater than about 61° C., greater than about 62° C., greater than about 63° C., greater than about 64° C., greater than about 65° C., greater than about 66° C., or greater than about 67° C. In certain embodiments, the T_{agg} is changed by less than about 2° C., less than 1.5° C., or less than about 1° C. when the pharmaceutical formulation is incubated for 1 week at 50° C., as compared to the same pharmaceutical formulation that is incubated for 1 week at 5° C. In certain embodiments, the T_{agg} is changed by less than about 2° C., less than about 1.5° C., or less than about 1° C. when the pharmaceutical formulation is incubated for 1 week at 50° C., as compared to the same pharmaceutical formulation that is incubated for 1 week at 5° C. In certain embodiments, the T_{agg} is changed by less than about 1° C. when the pharmaceutical formulation is incubated for 1 week at 50° C., as compared to the same pharmaceutical formulation that is incubated for 1 week at 5° C.

In some embodiments, pH is used to determine stability of the pharmaceutical formulation. In some embodiments, the pH of the pharmaceutical formulation does not change by more than about 0.25, about 0.2, about 0.15, or about 0.1 in pH value after the pharmaceutical formulation is incubated for 1 week at 5° C. In certain embodiments, the pH of the pharmaceutical formulation does not change by more than about 0.2 or about 0.1 in pH value after the pharmaceutical formulation is incubated for 1 week at 5° C. In some embodiments, the pH of the pharmaceutical formulation does not change by more than about 0.25, about 0.2, about 0.15, or about 0.1 in pH value after the pharmaceutical formulation is incubated for 1 week at 50° C. In certain embodiments, the pH of the pharmaceutical formulation does not change by more than about 0.2 or about 0.1 in pH value after the pharmaceutical formulation is incubated for 1 week at 50° C.

Exemplary methods to determine stability of the heterodimeric Fc-fused protein in the pharmaceutical formulation are described in Example 24 of the present disclosure.
(iv) Dosage Forms

The pharmaceutical formulation can be prepared and stored as a liquid formulation or a lyophilized form. In certain embodiments, the pharmaceutical formulation is a clear, colorless solution, free of visible particulates. In certain embodiments, the pharmaceutical formulation is a liquid formulation for storage at 2-8° C. (e.g., 4° C.), a frozen formulation for storage at −20° C. or lower, or a frozen formulation for storage at −65° C. or lower. The sugar and/or sugar alcohol in the formulation are used as lyoprotectants.

Prior to pharmaceutical use, the pharmaceutical formulation can be diluted or reconstituted in an aqueous carrier suitable for the route of administration. Other exemplary carriers include sterile water for injection (SWFI), bacteriostatic water for injection (BWFI), a pH buffered solution (e.g., phosphate-buffered saline), sterile saline solution, Ringer's solution, or dextrose solution. For example, when the pharmaceutical formulation is prepared for administration, the pharmaceutical formulation can be diluted in a 0.9% sodium chloride (NaCl) solution. In specific embodiments, the pharmaceutical formulation the pharmaceutical formulation is diluted in a 0.9% sodium chloride (NaCl) solution comprising 0.01% polysorbate 80. In certain embodiments, the diluted pharmaceutical formulation is isotonic and suitable for administration by subcutaneous injection.

The pharmaceutical formulation comprises the heterodimeric Fc-fused protein at a concentration suitable for storage. In certain embodiments, the pharmaceutical formulation comprises the heterodimeric Fc-fused protein at a concentration of about 0.1-about 2 mg/mL, about 0.2-about 1.8 mg/mL, about 0.3-about 1.7 mg/mL, about 0.4-about 1.6 mg/mL, about 0.5-about 1.5 mg/mL, about 0.6-about 1.4 mg/mL, about 0.7-about 1.3 mg/mL, about 0.8-about 1.2 mg/mL, or about 0.9-about 1.1 mg/mL. In certain embodiments, the pharmaceutical formulation comprises the heterodimeric Fc-fused protein at a concentration of about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1 mg/mL, about 2 mg/mL, about 2.5 mg/mL, about 3 mg/mL, about 3.5 mg/mL, about 4 mg/mL, about 4.5 mg/mL, about 5 mg/mL, or about 10 mg/mL.

In certain embodiments, the pharmaceutical formulation comprises the heterodimeric Fc-fused protein at a bulk concentration of about 1 g/L to about 10 g/L, about 2 g/L to about 8 g/L, about 4 g/L to about 6 g/L, or about 5 g/L. In certain embodiments, the pharmaceutical formulation comprises the heterodimeric Fc-fused protein at a bulk concentration of about 1 g/L, about 2 g/L, about 3 g/L, about 4 g/L, about 5 g/L, about 6 g/L, about 7 g/L, about 8 g/L, about 9 g/L, or about 10 g/L. In specific embodiments, the pharmaceutical formulation comprises a bulk concentration of heterodimeric Fc-fused protein of about 5 g/L. In certain embodiments, the pharmaceutical formulation comprises a concentration for administration of heterodimeric Fc-fused protein of about 0.5 g/L-about 2 g/L, about 0.75 g/L-about 1.5 g/L, or about 0.9 g/L-about 1.1 g/L. In certain embodiments, the pharmaceutical formulation comprises a concentration for administration of heterodimeric Fc-fused protein of about 0.5 g/L, about 0.6 g/L, about 0.7 g/L, about 0.8 g/L, about 0.9 g/L, about 1 g/L, about 1.1 g/L, about 1.2 g/L, about 1.3 g/L, about 1.4 g/L, about 1.5 g/L, or about 2 g/L. In specific embodiments, the pharmaceutical formulation comprises a concentration for administration of heterodimeric Fc-fused protein of about 1 g/L.

In certain embodiments, the pharmaceutical formulation is packaged in a container (e.g., a vial, bag, pen, or syringe). In certain embodiments, the formulation may be a lyophilized formulation or a liquid formulation. In certain embodiments, the amount of heterodimeric Fc-fused protein in the container is suitable for administration as a single dose. In certain embodiments, the amount of heterodimeric Fc-fused protein in the container is suitable for administration in multiple doses. In certain embodiments, the pharmaceutical formulation comprises the heterodimeric Fc-fused protein at an amount of about 0.1 to about 10 mg. In certain embodiments, the pharmaceutical formulation comprises the heterodimeric Fc-fused protein at an amount of about 0-about 2 mg, about 0.2-about 1.8 mg, about 0.3-about 1.7 mg, about 0.4-about 1.6 mg, about 0.5-about 1.5 mg, about 0.6-about 1.4 mg, about 0.7-about 1.3 mg, about 0.8-about 1.2 mg, or about 0.9-about 1.1 mg. In certain embodiments, the pharmaceutical formulation comprises the heterodimeric Fc-fused protein at an amount of about 0.1 mg, about 0.2 mg, about 0.3 mg, about 0.4 mg, about 0.5 mg, about 1 mg, about 2 mg, about 3 mg, about 4 mg, about 5 mg, or about 10 mg. In specific embodiments, the pharmaceutical formulation comprises the heterodimeric Fc-fused protein, e.g., DF-hIL-12-Fc si, at an amount of about 1 mg.
(1) Dosage Regimens and Therapeutic Uses

In another aspect, the present disclosure provides a method for treating cancer, the method comprising administering to a subject in need thereof a heterodimeric Fc-fused protein disclosed herein (e.g., DF-hIL-12-Fc si) in an initial three-week treatment cycle on Day 1. In certain embodiments, the heterodimeric Fc-fused protein is administered to the subject only Day 1 in the initial three-week treatment cycle.

In certain embodiments, the method comprises administering to the subject in need thereof the heterodimeric Fc-fused protein in combination with an anti-PD-1 antibody, e.g., pembrolizumab, in an initial three-week treatment cycle on Day 1. In certain embodiments, the heterodimeric Fc-fused protein and anti-PD-1 antibody are administered to the subject only Day 1 in the initial three-week treatment cycle. In certain embodiments, administration of the PD-1 antibody precedes administration of the heterodimeric Fc-fused protein.

In certain embodiments, the method further comprises administering to the subject, after the initial treatment cycle, the heterodimeric Fc-fused protein in one or more subsequent three-week treatment cycles, wherein the heterodimeric Fc-fused protein is administered on Day 1 in each subsequent treatment cycle. The subsequent treatment cycles, in which the subject receives administration of the heterodimeric Fc-fused protein once every three weeks or once every four weeks, are designed to maintain a certain level of the heterodimeric Fc-fused protein in the subject. In certain embodiments, the subject receives administration of the heterodimeric Fc-fused protein once every three weeks, once every four weeks, once every five weeks, or once every six weeks. In certain embodiments, the subject receives administration of the heterodimeric Fc-fused protein once every six weeks, i.e., once every other treatment cycle. In certain embodiments, the subject receives at least 1, 2, 3, 4, 5, 6, 7, 8, 9, 10, 11, 12, 13, 14, or 15 subsequent treatment cycles. In certain embodiments, the subject receives subsequent treatment cycles until regression of the cancer (i.e., a complete response). In certain embodiments, the subject has advanced (i.e., unresectable or metastatic) melanoma. In certain embodiments, the subject has advanced (i.e., unresectable or metastatic) renal cell carcinoma.

In certain embodiments, the method further comprises administering to the subject, after the initial treatment cycle, the heterodimeric Fc-fused protein in one or more subsequent three-week treatment cycles, in combination with an anti-PD-1 antibody, e.g., pembrolizumab, wherein the heterodimeric Fc-fused protein and anti-PD-1 antibody are administered on Day 1 in each subsequent treatment cycle. The subsequent treatment cycles, in which the subject receives administration of the heterodimeric Fc-fused protein and anti-PD-1 antibody once every three weeks, are designed to maintain a certain level of the heterodimeric Fc-fused protein and anti-PD-1 antibody in the subject. In certain embodiments, the subject receives at least 1, 2, 3, 4, 5, 6, 7, 8, 9, 10, 11, 12, 13, 14, or 15 subsequent treatment cycles. In certain embodiments, administration of the anti-PD-1 antibody precedes administration of the heterodimeric Fc-fused protein. In certain embodiments, the subject receives subsequent treatment cycles until regression of the cancer (i.e., a complete response). In certain embodiments, the subject has advanced (i.e., unresectable or metastatic) urothelial carcinoma.

In certain embodiments, one or more doses in the initial and subsequent treatment cycles comprise the heterodimeric Fc-fused protein at an amount of 0.01-about 3 μg/kg, about 0.01-about 0.02 μg/kg, about 0.01-about 0.05 μg/kg, about 0.05-about 0.1 μg/kg, about 0.05-about 0.5 μg/kg, about 0.05-about 0.75 μg/kg, about 0.05-about 1 μg/kg, about 0.05-about 1.5 μg/kg, about 0.05-about 2 μg/kg, about 0.05-about 2.5 μg/kg, about 0.05-about 3 μg/kg, about 0.1-about 3 μg/kg, about 0.1-about 1 μg/kg, about 0.5-about 1 μg/kg, about 0.1-about 2 μg/kg, about 0.5-about 2 μg/kg, about 0.1-about 0.5 μg/kg, about 0.1-about 0.25 μg/kg, about 0.2-about 1 μg/kg, about 0.2-about 2 μg/kg, about 1-about 1.2 μg/kg, about 1-about 1.5 μg/kg, about 1-about 2 μg/kg, about 1-about 2.5 μg/kg, about 0.5-about 2.5 μg/kg, about 1-about 3 μg/kg, or about 0.5-about 3 μg/kg. In certain embodiments, one or more doses in the initial and subsequent treatment cycles comprise the heterodimeric Fc-fused protein at an amount selected from the group consisting of about 0.01 μg/kg, about 0.02 μg/kg, about 0.03 μg/kg, about 0.04 μg/kg, about 0.05 μg/kg, about 0.1 μg/kg, about 0.15 μg/kg, about 0.2 μg/kg, about 0.25 μg/kg, about 0.3 μg/kg, about 0.35 μg/kg, about 0.4 μg/kg, about 0.45 μg/kg, about 0.5 μg/kg, about 0.6 μg/kg, about 0.7 μg/kg, about 0.8 μg/kg, about 0.9 μg/kg, about 1 μg/kg, about 1.2 μg/kg, about 1.25 μg/kg, about 1.3 μg/kg, about 1.4 μg/kg, about 1.5 μg/kg, about 1.75 μg/kg, about 2 μg/kg, about 2.5 μg/kg, about 3 μg/kg, about 4 μg/kg, or about 5 μg/kg.

In certain embodiments, each of the doses in the initial and subsequent treatment cycles comprises the heterodimeric Fc-fused protein at an amount of 0.01-about 3 μg/kg, about 0.01-about 0.02 μg/kg, about 0.01-about 0.05 μg/kg, about 0.05-about 0.1 μg/kg, about 0.05-about 0.5 μg/kg, about 0.05-about 0.75 μg/kg, about 0.05-about 1 μg/kg, about 0.05-about 1.5 μg/kg, about 0.05-about 2 μg/kg, about 0.05-about 2.5 μg/kg, about 0.05-about 3 μg/kg, about 0.1-about 3 μg/kg, about 0.1-about 1 μg/kg, about 0.5-about 1 μg/kg, about 0.1-about 2 μg/kg, about 0.5-about 2 μg/kg, about 0.1-about 0.5 μg/kg, about 0.1-about 0.25 μg/kg, about 0.2-about 1 μg/kg, about 0.2-about 2 μg/kg, about 1-about 1.2 μg/kg, about 1-about 1.5 μg/kg, about 1-about 2 μg/kg, about 1-about 2.5 μg/kg, about 0.5-about 2.5 μg/kg, about 1-about 3 μg/kg, or about 0.5-about 3 μg/kg. In certain embodiments, each of the doses in the initial and subsequent treatment cycles comprises the heterodimeric Fc-fused protein at a same amount selected from the group consisting of about 0.01 μg/kg, about 0.02 μg/kg, about 0.03 μg/kg, about 0.04 μg/kg, about 0.05 μg/kg, about 0.1 μg/kg, about 0.15 μg/kg, about 0.2 μg/kg, about 0.25 μg/kg, about 0.3 μg/kg, about 0.35 μg/kg, about 0.4 μg/kg, about 0.45 μg/kg, about 0.5 μg/kg, about 0.6 μg/kg, about 0.7 μg/kg, about 0.8 μg/kg, about 0.9 μg/kg, about 1 μg/kg, about 1.2 μg/kg, about 1.25 μg/kg, about 1.3 μg/kg, about 1.4 μg/kg, about 1.5 μg/kg, about 1.75 μg/kg, about 2 μg/kg, about 2.5 μg/kg, about 3 μg/kg, about 4 μg/kg, or about 5 μg/kg.

In certain embodiments, each of the doses in the initial and subsequent treatment cycles comprises the heterodimeric Fc-fused protein at an amount 0.01-about 3 μg/kg, about 0.01-about 0.02 μg/kg, about 0.01-about 0.05 μg/kg, about 0.05-about 0.1 μg/kg, about 0.05-about 0.5 μg/kg, about 0.05-about 0.75 μg/kg, about 0.05-about 1 μg/kg, about 0.05-about 1.5 μg/kg, about 0.05-about 2 μg/kg, about 0.05-about 2.5 μg/kg, about 0.05-about 3 μg/kg, about 0.1-about 3 μg/kg, about 0.1-about 1 μg/kg, about 0.5-about 1 μg/kg, about 0.1-about 2 μg/kg, about 0.5-about 2 μg/kg, about 0.1-about 0.5 μg/kg, about 0.1-about 0.25 μg/kg, about 0.2-about 1 μg/kg, about 0.2-about 2 μg/kg, about 1-about 1.2 μg/kg, about 1-about 1.5 μg/kg, about 1-about 2 μg/kg, about 1-about 2.5 μg/kg, about 0.5-about 2.5 μg/kg, about 1-about 3 μg/kg, or about 0.5-about 3 μg/kg. In certain embodiments, each of the doses in the initial and subsequent treatment cycles comprises the heterodimeric Fc-fused protein at a same amount selected from the group consisting of about 0.01 μg/kg, about 0.02 μg/kg, about 0.03 μg/kg, about 0.04 μg/kg, about 0.05 μg/kg, about 0.1 μg/kg, about 0.15 μg/kg, about 0.2 μg/kg, about 0.25 μg/kg, about 0.3 μg/kg, about 0.35 μg/kg, about 0.4 μg/kg, about 0.45 μg/kg, about 0.5 μg/kg, about 0.6 μg/kg, about 0.7 μg/kg, about 0.8 μg/kg, about 0.9 μg/kg, about 1 μg/kg, about 1.2 μg/kg, about 1.25 μg/kg, about 1.3 μg/kg, about 1.4 μg/kg, about 1.5 μg/kg, about 1.75 μg/kg, about 2 μg/kg, about 2.5 μg/kg, about 3 μg/kg, about 4 μg/kg, or about 5 μg/kg.

In certain embodiments, the heterodimeric Fc-fused protein is administered subcutaneously. For example, in certain embodiments, the heterodimeric Fc-fused protein is administered by subcutaneous injection, e.g., with a prefilled pen or a prefilled syringe. In certain embodiments, the heterodimeric Fc-fused protein is administered in a volume of about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.1 mL, or about 1.2 mL. In certain embodiments, the heterodimeric Fc-fused protein is administered in a volume of about 1 mL. In certain embodiments, the heterodimeric Fc-fused protein is administered in a maximum of 2 injection sites (e.g., 1 injection site, or 2 injection sites). In specific embodiments, the heterodimeric Fc-fused protein is administered in a single injection. In specific embodiments, the heterodimeric Fc-fused protein is administered in two injections. In specific embodiments, the heterodimeric Fc-fused protein is administered in two injections, and a second injection is completed within 10 minutes after a first injection.

In certain embodiments, the anti-PD-1 antibody, e.g., pembrolizumab, is administered intravenously. In certain embodiments, the anti-PD-1 antibody is administered intravenously preceding administration of the heterodimeric Fc-fused protein. In certain embodiments, the anti-PD-1 antibody is administered intravenously no more than 1 hour (e.g., 5 mins, 10 mins, 15 mins, 20 mins, 25 mins, 30 mins, 35 mins, 40 mins, 45 mins, 50 mins, 55 mins, 1 hour) prior to administration of the heterodimeric Fc-fused protein. In certain embodiments, the PD-1 antibody is administered intravenously concurrently with administration of the heterodimeric Fc-fused protein.

The types of cancer that can be treated with the heterodimeric Fc-fused protein or pharmaceutical formulation disclosed herein include but are not limited to melanoma, non-small cell lung cancer (NSCLC), small-cell lung cancer (SCLC), head and neck squamous cell carcinoma (HNSCC), classical Hodgkin lymphoma, primary mediastinal large B-Cell lymphoma, bladder cancer, urothelial carcinoma, micro-satellite instability high cancer, colorectal cancer, gastric cancer, oesophageal cancer, cervical cancer, hepatocellular carcinoma, Merkel cell carcinoma, renal cell carcinoma (RCC), endometrial carcinoma, cutaneous T cell lymphoma, or triple negative breast cancer. In certain embodiments, the cancer is a solid tumor. In certain embodiments, the cancer is a locally advanced or metastatic solid tumor. In certain embodiments, the cancer is melanoma. In certain embodiments, the cancer is renal cell carcinoma. In certain embodiments, the cancer is urothelial bladder cancer. In certain embodiments, the subject has clinical or radiological evidence of disease. In certain embodiments, the subject has measurable disease, as determined by the Response Evaluation Criteria for Solid Tumors (RECIST), version 1.1. In certain embodiments, the pharmaceutical formulation disclosed herein is administered as a monotherapy. In certain embodiments, the pharmaceutical formulation disclosed herein is administered as a combination therapy. In certain embodiments, a subject who has a confirmed complete response (CR) is treated with the pharmaceutical formulation for at least 12 months after confirmation, unless a criterion for discontinuation is met. In certain embodiments, the total duration of the multi-dose therapy is equal to or less than 24 months (e.g., 1 month, 2 months, 3 months, 4 months, 5 months, 6 months, 12 months, 18 months, 24 months). In certain embodiments, the total duration of the multi-dose therapy is more than 24 months.

In certain embodiments, the subject treated by the method disclosed herein has advanced melanoma. In certain embodiments, the subject has received treatment with an anti PD-1 antibody for at least 6 weeks and has confirmed disease progression. In certain embodiments, the subject has a BRAF activating mutation, has received a BRAF inhibitor, and has disease progression after the last line of treatment. In certain embodiments, progressive disease is confirmed by radiological or clinical observation. In certain embodiments, the subject does not have a BRAF activating mutation.

In certain embodiments, the subject treated by the method disclosed herein has advanced renal clear cell carcinoma (RCC). In certain embodiments, the subject has a clear cell histology component. In certain embodiments, the subject has received treatment with a checkpoint inhibitor, e.g., an anti PD-1/PD-L1 antibody, or a VEGF therapy as a monotherapy. In certain embodiments, the subject has received treatment with a checkpoint inhibitor, e.g., an anti PD-1/PD-L1 antibody and a VEGF therapy in combination. In certain embodiments, the subject has received treatment with a checkpoint inhibitor, e.g., an anti PD-1/PD-L1 antibody, and a platinum-based chemotherapy in combination. In certain embodiments, the subject has not received treatment with a checkpoint inhibitor, e.g., an anti PD-1/PD-L1 antibody. In certain embodiments, the subject has received more than 3 prior lines of therapy.

In certain embodiments, the subject treated by the method disclosed herein has advanced urothelial carcinoma. In certain embodiments, the advanced urothelial carcinoma is metastatic or unresectable. In certain embodiments, the subject has histologically or cytologically documented locally advanced or metastatic transitional cell carcinoma of the urothelium (including but not limited to the renal pelvis, ureters, urinary urothelial, and urethra). In certain embodiments, the subject has received only one platinum-containing regimen (e.g., platinum plus another agent, such as gemcitabine, methotrexate, vinblastine, doxorubicin, etc.). In some embodiments, the subject has not received more than one platinum-containing regimen for inoperable locally advanced or metastatic urothelial carcinoma with radiographic progression or with recurrence within 6 months after the last administration of the platinum-containing regimen as an adjuvant In certain embodiments, the subject has not received treatment with a checkpoint inhibitor (CPI) (e.g., anti-PD-1 or anti-PD-L1) as a monotherapy, or in combination with a platinum based chemotherapy. In certain embodiments, the subject has received 2 prior lines of therapy. In certain embodiments, the urothelial carcinoma is considered failure of a first-line, platinum-containing regimen.

In some embodiments, the drug delivery formulation for use in a method of treating cancer or inhibiting tumor growth comprises administration of the heterodimeric Fc-fused protein at a dose of about 0.01-about 3 μg/kg, about 0.01-about 0.02 μg/kg, about 0.01-about 0.05 μg/kg, about 0.05-about 0.1 μg/kg, about 0.05-about 0.5 μg/kg, about 0.05-about 0.75 μg/kg, about 0.05-about 1 μg/kg, about 0.05-about 1.5 μg/kg, about 0.05-about 2 μg/kg, about 0.05-about 2.5 μg/kg, about 0.05-about 3 μg/kg, about 0.1-about 3 μg/kg, about 0.1-about 1 μg/kg, about 0.5-about 1 μg/kg, about 0.1-about 2 μg/kg, about 0.5-about 2 μg/kg, about 0.1-about 0.5 μg/kg, about 0.1-about 0.25 μg/kg, about 0.2-about 1 μg/kg, about 0.2-about 2 μg/kg, about 1-about 1.2 μg/kg, about 1-about 1.5 μg/kg, about 1-about 2 μg/kg, about 1-about 2.5 μg/kg, about 0.5-about 2.5 μg/kg, about 1-about 3 μg/kg, or about 0.5-about 3 μg/kg. In certain embodiments, the drug delivery formulation for use in a method of treating cancer or inhibiting tumor growth comprises administration of the heterodimeric Fc-fused protein at a dose selected from the group consisting of about 0.01 μg/kg, about 0.02 μg/kg, about 0.03 μg/kg, about 0.04 μg/kg, about 0.05 μg/kg, about 0.1 μg/kg, about 0.15 μg/kg, about 0.2 μg/kg, about 0.25 μg/kg, about 0.3 μg/kg, about 0.35 μg/kg, about 0.4 μg/kg, about 0.45 μg/kg, about 0.5 μg/kg, about 0.6 μg/kg, about 0.7 μg/kg, about 0.8 μg/kg, about 0.9 μg/kg, about 1 μg/kg, about 1.2 μg/kg, about 1.25 μg/kg, about 1.3 μg/kg, about 1.4 μg/kg, about 1.5 μg/kg, about 1.75 μg/kg, about 2 μg/kg, about 2.5 μg/kg, about 3 μg/kg, about 4 μg/kg, or about 5 μg/kg. In certain embodiments, the dose administered is based on the subject's weight. In certain embodiments, the drug delivery formulation for use in a method of treating cancer or inhibiting tumor growth further comprises administering an anti-PD-1 antibody, e.g., pembrolizumab or nivolumab.

In some embodiments, the drug delivery formulation for use in a method of treating cancer or inhibiting tumor growth comprises the heterodimeric Fc-fused protein at an amount of about 0-about 2 mg, about 0.2-about 1.8 mg, about 0.3-about 1.7 mg, about 0.4-about 1.6 mg, about 0.5-about 1.5 mg, about 0.6-about 1.4 mg, about 0.7-about 1.3 mg, about 0.8-about 1.2 mg, or about 0.9-about 1.1 mg. In certain embodiments, the drug delivery formulation for use in a method of treating cancer or inhibiting tumor growth comprises the heterodimeric Fc-fused protein at an amount selected from the group consisting of about 0.1 mg, about 0.2 mg, about 0.3 mg, about 0.4 mg, about 0.5 mg, about 1 mg, about 2 mg, about 3 mg, about 4 mg, about 5 mg, or about 10 mg. In certain embodiments, the drug delivery formulation for use in a method of treating cancer or inhibiting tumor growth further comprises administering an anti-PD-1 antibody, e.g., pembrolizumab or nivolumab. In specific embodiments, the drug delivery formulation for use in a method of treating cancer or inhibiting tumor growth further comprises administering 200 mg of an anti-PD-1 antibody, e.g., pembrolizumab or nivolumab.

In some embodiments, the drug delivery formulation for use in a method of treating cancer or inhibiting tumor growth in a subject with advanced melanoma is administered at a dose of about 0.01-about 3 μg/kg, about 0.01-about 0.02 μg/kg, about 0.01-about 0.05 μg/kg, about 0.05-about 0.1 μg/kg, about 0.05-about 0.5 μg/kg, about 0.05-about 0.75 μg/kg, about 0.05-about 1 μg/kg, about 0.05-about 1.5 μg/kg, about 0.05-about 2 μg/kg, about 0.05-about 2.5 μg/kg, about 0.05-about 3 μg/kg, about 0.1-about 3 μg/kg, about 0.1-about 1 μg/kg, about 0.5-about 1 μg/kg, about 0.1-about 2 μg/kg, about 0.5-about 2 μg/kg, about 0.1-about 0.5 μg/kg, about 0.1-about 0.25 μg/kg, about 0.2-about 1 μg/kg, about 0.2-about 2 μg/kg, about 1-about 1.2 μg/kg, about 1-about 1.5 μg/kg, about 1-about 2 μg/kg, about 1-about 2.5 μg/kg, about 0.5-about 2.5 μg/kg, about 1-about 3 μg/kg, or about 0.5-about 3 μg/kg. In certain embodiments, the drug delivery formulation for use in a method of treating cancer or inhibiting tumor growth in a subject with advanced melanoma is administered a dose selected from the group consisting of about 0.01 μg/kg, about 0.02 μg/kg, about 0.03 μg/kg, about 0.04 μg/kg, about 0.05 μg/kg, about 0.1 μg/kg, about 0.15 μg/kg, about 0.2 μg/kg, about 0.25 μg/kg, about 0.3 μg/kg, about 0.35 μg/kg, about 0.4 μg/kg, about 0.45 μg/kg, about 0.5 μg/kg, about 0.6 μg/kg, about 0.7 μg/kg, about 0.8 μg/kg, about 0.9 μg/kg, about 1 μg/kg, about 1.2 μg/kg, about 1.25 μg/kg, about 1.3 μg/kg, about 1.4 μg/kg, about 1.5 μg/kg, about 1.75 μg/kg, about 2 μg/kg, about 2.5 μg/kg, about 3 μg/kg, about 4 μg/kg, or about 5 μg/kg.

In some embodiments, the drug delivery formulation for use in a method of treating cancer or inhibiting tumor growth in a subject with advanced melanoma comprises the heterodimeric Fc-fused protein at an amount of about 0-about 2 mg, about 0.2-about 1.8 mg, about 0.3-about 1.7 mg, about 0.4-about 1.6 mg, about 0.5-about 1.5 mg, about 0.6-about 1.4 mg, about 0.7-about 1.3 mg, about 0.8-about 1.2 mg, or about 0.9-about 1.1 mg. In certain embodiments, the drug delivery formulation for use in a method of treating cancer or inhibiting tumor growth in a subject with advanced melanoma comprises the heterodimeric Fc-fused protein at an amount selected from the group consisting of about 0.1 mg, about 0.2 mg, about 0.3 mg, about 0.4 mg, about 0.5 mg, about 1 mg, about 2 mg, about 3 mg, about 4 mg, about 5 mg, or about 10 mg.

In some embodiments, the drug delivery formulation for use in a method of treating cancer or inhibiting tumor growth in a subject with advanced RCC is administered at a dose of about 0.01-about 3 μg/kg, about 0.01-about 0.02 μg/kg, about 0.01-about 0.05 μg/kg, about 0.05-about 0.1 μg/kg, about 0.05-about 0.5 μg/kg, about 0.05-about 0.75 μg/kg, about 0.05-about 1 μg/kg, about 0.05-about 1.5 μg/kg, about 0.05-about 2 μg/kg, about 0.05-about 2.5 μg/kg, about 0.05-about 3 μg/kg, about 0.1-about 3 μg/kg, about 0.1-about 1 μg/kg, about 0.5-about 1 μg/kg, about 0.1-about 2 μg/kg, about 0.5-about 2 μg/kg, about 0.1-about 0.5 μg/kg, about 0.1-about 0.25 μg/kg, about 0.2-about 1 μg/kg, about 0.2-about 2 μg/kg, about 1-about 1.2 μg/kg, about 1-about 1.5 μg/kg, about 1-about 2 μg/kg, about 1-about 2.5 μg/kg, about 0.5-about 2.5 μg/kg, about 1-about 3 μg/kg, or about 0.5-about 3 μg/kg. In certain embodiments, the drug delivery formulation for use in a method of treating cancer or inhibiting tumor growth in a subject with advanced RCC is administered a dose selected from the group consisting of about 0.01 μg/kg, about 0.02 μg/kg, about 0.03 μg/kg, about 0.04 μg/kg, about 0.05 μg/kg, about 0.1 μg/kg, about 0.15 μg/kg, about 0.2 μg/kg, about 0.25 μg/kg, about 0.3 μg/kg, about 0.35 μg/kg, about 0.4 μg/kg, about 0.45 μg/kg, about 0.5 μg/kg, about 0.6 μg/kg, about 0.7 μg/kg, about 0.8 μg/kg, about 0.9 μg/kg, about 1 μg/kg, about 1.2 μg/kg, about 1.25 μg/kg, about 1.3 μg/kg, about 1.4 μg/kg, about 1.5 μg/kg, about 1.75 μg/kg, about 2 μg/kg, about 2.5 μg/kg, about 3 μg/kg, about 4 μg/kg, or about 5 μg/kg.

In some embodiments, the drug delivery formulation for use in a method of treating cancer or inhibiting tumor growth in a subject with advanced RCC comprises the heterodimeric Fc-fused protein at an amount of about 0-about 2 mg, about 0.2-about 1.8 mg, about 0.3-about 1.7 mg, about 0.4-about 1.6 mg, about 0.5-about 1.5 mg, about 0.6-about 1.4 mg, about 0.7-about 1.3 mg, about 0.8-about 1.2 mg, or about 0.9-about 1.1 mg. In certain embodiments, the drug delivery formulation for use in a method of treating cancer or inhibiting tumor growth in a subject with advanced RCC comprises the heterodimeric Fc-fused protein at an amount selected from the group consisting of about 0.1 mg, about 0.2 mg, about 0.3 mg, about 0.4 mg, about 0.5 mg, about 1 mg, about 2 mg, about 3 mg, about 4 mg, about 5 mg, or about 10 mg.

In some embodiments, the drug delivery formulation for use in a method of treating cancer or inhibiting tumor growth in a subject with advanced urothelial carcinoma is administered at a dose of about 0.01-about 3 μg/kg, about 0.01-about 0.02 μg/kg, about 0.01-about 0.05 μg/kg, about 0.05-about 0.1 μg/kg, about 0.05-about 0.5 μg/kg, about 0.05-about 0.75 μg/kg, about 0.05-about 1 μg/kg, about 0.05-about 1.5 μg/kg, about 0.05-about 2 μg/kg, about 0.05-about 2.5 μg/kg, about 0.05-about 3 μg/kg, about 0.1-about 3 μg/kg, about 0.1-about 1 μg/kg, about 0.5-about 1 μg/kg, about 0.1-about 2 μg/kg, about 0.5-about 2 μg/kg, about 0.1-about 0.5 μg/kg, about 0.1-about 0.25 μg/kg, about 0.2-about 1 μg/kg, about 0.2-about 2 μg/kg, about 1-about 1.2 μg/kg, about 1-about 1.5 μg/kg, about 1-about 2 μg/kg, about 1-about 2.5 μg/kg, about 0.5-about 2.5 μg/kg, about 1-about 3 μg/kg, or about 0.5-about 3 μg/kg. In certain embodiments, the drug delivery formulation for use in a method of treating cancer or inhibiting tumor growth in a subject with advanced urothelial carcinoma is administered a dose selected from the group consisting of about 0.01 μg/kg, about 0.02 μg/kg, about 0.03 μg/kg, about 0.04 μg/kg, about 0.05 μg/kg, about 0.1 μg/kg, about 0.15 μg/kg, about 0.2 μg/kg, about 0.25 μg/kg, about 0.3 μg/kg, about 0.35 μg/kg, about 0.4 μg/kg, about 0.45 μg/kg, about 0.5 μg/kg, about 0.6 μg/kg, about 0.7 μg/kg, about 0.8 μg/kg, about 0.9 μg/kg, about 1 μg/kg, about 1.2 μg/kg, about 1.25 μg/kg, about 1.3 μg/kg, about 1.4 μg/kg, about 1.5 μg/kg, about 1.75 μg/kg, about 2 μg/kg, about 2.5 μg/kg, about 3 μg/kg, about 4 μg/kg, or about 5 μg/kg.

In some embodiments, the drug delivery formulation for use in a method of treating cancer or inhibiting tumor growth in a subject with advanced urothelial carcinoma comprises the heterodimeric Fc-fused protein at an amount of about 0-about 2 mg, about 0.2-about 1.8 mg, about 0.3-about 1.7 mg, about 0.4-about 1.6 mg, about 0.5-about 1.5 mg, about 0.6-about 1.4 mg, about 0.7-about 1.3 mg, about 0.8-about 1.2 mg, or about 0.9-about 1.1 mg. In certain embodiments, the drug delivery formulation for use in a method of treating cancer or inhibiting tumor growth in a subject with advanced urothelial carcinoma comprises the heterodimeric Fc-fused protein at an amount selected from the group consisting of about 0.1 mg, about 0.2 mg, about 0.3 mg, about 0.4 mg, about 0.5 mg, about 1 mg, about 2 mg, about 3 mg, about 4 mg, about 5 mg, or about 10 mg.

In certain embodiments, the subject treated in accordance with the methods disclosed herein has not received prior therapy for treating the cancer. In certain embodiments, the subject treated in accordance with the methods disclosed herein has not received prior chemotherapy or immunotherapy for treating the cancer. In certain embodiments, the subject treated in accordance with the methods disclosed herein has received a prior therapy (e.g., a chemotherapy or immunotherapy) but continues to experience cancer progression despite the prior therapy. In certain embodiments, the subject treated in accordance with the methods disclosed herein has experienced cancer regression after receiving a prior therapy (e.g., a chemotherapy or immunotherapy), but later experienced cancer relapse. In certain embodiments, the subject treated in accordance with the methods disclosed herein is intolerant to a prior therapy (e.g., a chemotherapy or immunotherapy).

In certain embodiments, the subject treated in accordance with the methods disclosed herein meets all the inclusion criteria of a clinical trial cohort (e.g., the dose escalation cohort, the dose expansion cohorts, the melanoma cohort, the renal cell carcinoma cohort, the urothelial carcinoma cohort, or the combination therapy with pembrolizumab or nivolumab cohorts) described in Examples 26 and 29. In certain embodiments, the subject treated in accordance with the methods disclosed herein does not meet any of the exclusion criteria described in Examples 26 and 29.

The heterodimeric Fc-fused protein disclosed herein can be used as a monotherapy or in combination with one or more therapies. In certain embodiments, the heterodimeric Fc-fused protein is used as a monotherapy in accordance with the dosage regimen disclosed herein. In other embodiments, the heterodimeric Fc-fused protein is used in combination with one or more therapies, wherein the heterodimeric Fc-fused protein is administered in accordance with a dosage regimen disclosed herein and the one or more therapies are administered in accordance with a dosage regimen known to be suitable for treating the particular subject with the particular cancer. In certain embodiments, the method of treatment disclosed herein is used as an adjunct to surgical removal of the primary lesion. In certain embodiments, a surgical intervention of the primary lesion comprises lysing cancer cells, removing a tumor, or debulking a tumor in the subject.

Exemplary therapeutic agents that may be used in combination with the heterodimeric Fc-fused protein include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma (IFN-7), colony stimulating factor-1, colony stimulating factor-2, denileukin diftitox, interleukin-2, luteinizing hormone releasing factor and variations of the aforementioned agents that may exhibit differential binding to their cognate receptors, or increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T lymphocyte-associated antigen 4 (CTLA4), (ii) programmed cell death protein 1 (PD1), (iii) PDL1, (iv) LAG3, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

In certain embodiments, the cancer treated with a single dose or more of a heterodimeric IL-12-Fc-fused protein (e.g., comprising a first polypeptide comprising the amino acid sequence of SEQ ID NO:290 and a second polypeptide comprising the amino acid sequence of SEQ ID NO:291) is a metastatic cancer. In certain embodiments, the metastatic cancer is a local, regional, or distant metastatic cancer. In certain embodiments, a single or multiple dose of a heterodimeric IL-12-Fc-fused protein (e.g., comprising a first polypeptide comprising the amino acid sequence of SEQ ID NO:290 and a second polypeptide comprising the amino acid sequence of SEQ ID NO:291) treats a distant cancer, which is not the primary cancer of the source organ or tissue and/or the direct target of a treatment regimen, by an abscopal effect. In certain embodiments the abscopal effect of a heterodimeric IL-12-Fc-fused protein is enhanced during and/or after a treatment plan including radiation and/or chemotherapy. In certain embodiments, a single or multiple dose of a heterodimeric IL-12-Fc-fused protein (e.g., comprising a first polypeptide comprising the amino acid sequence of SEQ ID NO:290 and a second polypeptide comprising the amino acid sequence of SEQ ID NO:291) treats cancer in a patient by inducing a systemic anti-tumor response, determined, for example, by increased expression of IFNγ, CXCL9, and/or CXCL10 in the serum and/or the tumor of the patient.

### Combination Therapy

Another aspect of the invention provides for combination therapy. A pharmaceutical formulation comprising a heterodimeric Fc fused protein described herein can be used in combination with additional therapeutic agents to treat the cancer.

In certain embodiments, the heterodimeric Fc-fused protein of the present invention (e.g., a heterodimeric Fc-fused protein comprising IL-12 subunits) is administered as a combination therapy to treat a subject diagnosed with cancer. In certain embodiments, the cancer is bladder cancer, breast cancer, cervical cancer, colorectal cancer, oesophageal cancer, gastric cancer, head and neck cancer, hepatocellular carcinoma, leukemia, lung cancer, lymphoma, mesothelioma, melanoma, myeloma, ovarian cancer, endometrial carcinoma, prostate cancer, pancreatic cancer, renal cell carcinoma (RCC), non-small cell lung cancer (NSCLC), small cell lung cancer (SCLC), brain cancer, sarcoma, neuroblastoma, classical Hodgkin lymphoma, primary mediastinal large B-Cell lymphoma, urothelial carcinoma, micro-satellite instability high cancer, Merkel cell carcinoma, endometrial carcinoma, cutaneous T cell lymphoma, triple negative breast cancer, or head and neck squamous cell carcinoma (HNSCC). In certain embodiments, the cancer is colon cancer. In certain embodiments, the heterodimeric Fc-fused protein is administered as a combination therapy to a subject diagnosed with colon cancer. In certain embodiments, the cancer is melanoma. In certain embodiments, the heterodimeric Fc-fused protein is administered as a combination therapy to a subject diagnosed with melanoma. In certain embodiments, the cancer is breast cancer. In certain embodiments, the heterodimeric Fc-fused protein is administered as a combination therapy to a subject diagnosed with breast cancer.

In some embodiments, a heterodimeric Fc-fused protein of the present invention (e.g., a heterodimeric Fc-fused protein comprising IL-12 subunits) is used in treating an advanced malignancy in combination with another therapeutic agent selected from: cytotoxic chemotherapy; radiotherapy; an antibody that targets a molecule involved in an anti-tumor immune response, such as CTLA-4, PD-1, PD-L1, or TGF-β; an antibody that acts by ADCC on a tumor-associated antigen; a multispecific antibody binding NKG2D, CD16, and a tumor-associated antigen, optionally administered in combination with an antibody that targets PD-1 or PD-L1; a personalized cancer vaccine; an oncolytic cancer vaccine; and a personalized vaccine administered in combination with an antibody that targets PD-1 or PD-L1.

In some embodiments, a heterodimeric Fc-fused protein of the present invention (e.g., a heterodimeric Fc-fused protein comprising IL-12 subunits) is used in treating malignancy (e.g., an advanced malignancy) in combination with another therapy including, but not limited to, an NK-targeting therapy (e.g., CAR-NK therapy), an antibody therapy, a checkpoint inhibitor therapy, an additional cytokine therapy, an innate immune system agonist therapy, a chemotherapy, a target agent therapy, a radiotherapy, an adoptive NK therapy, a stem cell transplant (SCT) therapy, an agonistic antibody, a chimeric antigen receptor (CAR) T cell therapy, a T-cell receptor (TCR) engineered therapy, a multi-specific binding protein (TriNKET), an agent that induces cellular senescence, and a vaccine and/or oncolytic virus therapy. In some embodiments, a heterodimeric Fc-fused protein of the present invention is used in treating malignancy (e.g., an advanced malignancy) in combination with two or more additional therapies selected from an NK-targeting therapy (e.g., CAR-NK therapy), an antibody therapy, a checkpoint inhibitor therapy, an additional cytokine therapy, an innate immune system agonist therapy, a chemotherapy, a target agent therapy, a radiotherapy, an adoptive NK therapy, a stem cell transplant (SCT) therapy, an agonistic antibody, a chimeric antigen receptor (CAR) T cell therapy, a T-cell receptor (TCR) engineered therapy, a multi-specific binding protein (TriNKET), an agent that induces cellular senescence, and a vaccine and/or oncolytic virus therapy.

In some embodiments, a heterodimeric Fc-fused protein of the present invention (e.g., a heterodimeric Fc-fused protein comprising IL-12) is used in treating locally advanced malignancy that can be fully resected, in combination with a cancer vaccine or an antibody that targets PD-1 or PD-L1.

Proteins of the invention can also be used as an adjunct to surgical removal of the primary lesion.

The amount of heterodimeric Fc-fused protein of the present invention (e.g., a heterodimeric Fc-fused protein comprising IL-12) and additional therapeutic agent and the relative timing of administration may be selected in order to achieve a desired combined therapeutic effect. For example, when administering a combination therapy to a patient in need of such administration, the therapeutic agents in the combination, a pharmaceutical formulation or formulations comprising the therapeutic agents, or a pharmaceutical composition or compositions comprising the therapeutic agents, may be administered in any order such as, for example, sequentially, concurrently, together, simultaneously and the like. Further, for example, a heterodimeric Fc-fused protein may be administered during a time when the additional therapeutic agent(s) exerts its prophylactic or therapeutic effect, or vice versa.

As disclosed herein, the methods of the invention include coadministration of the combination of a heterodimeric Fc-fused protein (e.g., a heterodimeric Fc-fused protein comprising IL-12 subunits) and an additional therapeutic agent. As disclosed herein, the methods of the invention include coadministration of the combination of a heterodimeric Fc-fused protein comprising IL-12 subunits and an additional therapeutic agent.

“Coadministered” encompasses methods where a heterodimeric Fc-fused protein (e.g., a heterodimeric Fc-fused protein comprising IL-12 subunits) and an additional therapeutic agent are given simultaneously, where a heterodimeric Fc-fused protein and an additional therapeutic agent are given sequentially, and where either one of, or both of, a heterodimeric Fc-fused protein and an additional therapeutic agent are given intermittently or continuously, or any combination of: simultaneously, sequentially, intermittently and/or continuously. The skilled artisan will recognize that intermittent administration is not necessarily the same as sequential because intermittent also includes a first administration of an agent and then another administration later in time of that very same agent. Moreover, the skilled artisan understands that intermittent administration also encompasses sequential administration in some embodiments because intermittent administration does include interruption of the first administration of an agent with an administration of a different agent before the first agent is administered again. Further, the skilled artisan will also know that continuous administration can be accomplished by a number of routes including intravenous drip (IV infusion) or feeding tubes, etc.

Furthermore, and in a more general way, the term “coadministered” encompasses any and all methods where the individual administration of a heterodimeric Fc-fused protein and the individual administration of an additional therapeutic agent to a subject overlap during any timeframe.

The frequency of administration of a heterodimeric Fc-fused protein or an additional therapeutic agent to a subject is known in the art as Qnd or qnd where n is the frequency in days for successive administration of that agent. For example, Q3d would be an administration of an agent once every three (3) days. In certain embodiments, the method comprises administering either one of, or both of, or any combinations thereof, a heterodimeric Fc-fused protein and/or an additional therapeutic agent to a subject for Q1d, Q2d, Q3d, Q4d, Q5d, Q6d, Q7d, Q8d, Q9d, Q10d, Q14d, Q21d, Q28d, Q30d, Q90d, Q120d, Q240d, or Q365d.

In certain embodiments, either one of or both of a heterodimeric Fc-fused protein and/or an additional therapeutic agent are administered intermittently. In certain embodiments, the method includes administering either one of, or both of a heterodimeric Fc-fused protein or an additional therapeutic agent to a subject with a delay of at least 10 minutes, 15 minutes, 20 minutes, 30 minutes, 40 minutes, 60 minutes, 2 hours, 3 hours, 4 hours, 6 hours, 8 hours, 10 hours, 12 hours, 14 hours, 18 hours, 24 hours, 36 hours, 48 hours, 3 days, 4 days, 5 days, 6 days, 7 days, 8 days, 9 days, 10 days, 11 days, 12 days, 13 days, 14 days, 3 weeks, or 4 weeks between administrations. In certain embodiments, the administration with a delay follows a pattern where one of, or both of, or any combination thereof, of a heterodimeric Fc-fused protein and/or an additional therapeutic agent are administered continuously for a given period of time from about 10 minutes to about 365 days and then is not administered for a given period of time from about 10 minutes to about 30 days.

In certain embodiments, either one of, or any combination of, a heterodimeric Fc-fused protein and/or an additional therapeutic agent are administered intermittently while the other is given continuously. In certain embodiments, the combination of the first effective amount of a heterodimeric Fc-fused protein is administered sequentially with the second effective amount of an additional therapeutic agent.

In certain embodiments, a heterodimeric Fc-fused protein and an additional therapeutic agent are administered simultaneously. In certain embodiments, the combination of the first effective amount of a heterodimeric Fc-fused protein is administered sequentially with the second effective amount of an additional therapeutic agent. In such embodiments, the combination is also said to be “coadministered” since the term includes any and all methods where the subject is exposed to both components in the combination. However, such embodiments are not limited to the combination being given just in one formulation or composition. It may be that certain concentrations of a heterodimeric Fc-fused protein and the additional therapeutic agent are more advantageous to deliver at certain intervals and as such, the first effective amount and second effective amount may change according to the formulation being administered.

In certain embodiments, a heterodimeric Fc-fused protein and the additional therapeutic agent are administered simultaneously or sequentially. In certain embodiments, the first effective amount of a heterodimeric Fc-fused protein is administered sequentially after the second effective amount of an additional therapeutic agent. In certain embodiments, the second effective amount of an additional therapeutic agent is administered sequentially after the first effective amount of a heterodimeric Fc-fused protein.

In certain embodiments, the combination of a heterodimeric Fc-fused protein (e.g., a heterodimeric Fc-fused protein comprising IL-12 subunits) and an additional therapeutic agent is administered in one formulation. In certain embodiments, the combination is administered in two (2) compositions where the first effective amount of a heterodimeric Fc-fused protein is administered in a separate formulation from the formulation of the second effective amount of an additional therapeutic agent. In certain embodiments, the combination is administered in two (2) compositions where the first effective amount of the heterodimeric Fc-fused protein is administered in a separate formulation from the formulation of the second effective amount of an additional therapeutic agent. In certain embodiments, the first effective amount of a heterodimeric Fc-fused protein is administered sequentially after the second effective amount of an additional therapeutic agent. In certain embodiments, the second effective amount of an additional therapeutic agent is administered sequentially after the first effective amount of a heterodimeric Fc-fused protein. In certain embodiments, a heterodimeric Fc-fused protein and the additional therapeutic agent are administered; and subsequently both the heterodimeric Fc-fused protein and the additional therapeutic agent are administered intermittently for at least 24 hours. In certain embodiments, the heterodimeric Fc-fused protein and the additional therapeutic agent are administered on a non-overlapping every other day schedule.

In certain embodiments, the first effective amount of a heterodimeric Fc-fused protein is administered no less than 4 hours after the second effective amount of an additional therapeutic agent. In certain embodiments, the first effective amount of a heterodimeric Fc-fused protein is administered no less than 10 minutes, no less than 15 minutes, no less than 20 minutes, no less than 30 minutes, no less than 40 minutes, no less than 60 minutes, no less than 1 hour, no less than 2 hours, no less than 4 hours, no less than 6 hours, no less than 8 hours, no less than 10 hours, no less than 12 hours, no less than 24 hours, no less than 2 days, no less than 4 days, no less than 6 days, no less than 8 days, no less than 10 days, no less than 12 days, no less than 14 days, no less than 21 days, or no less than 30 days after the second effective amount of an additional therapeutic agent. In In certain embodiments, the second effective amount of an additional therapeutic agent is administered no less than 10 minutes, no less than 15 minutes, no less than 20 minutes, no less than 30 minutes, no less than 40 minutes, no less than 60 minutes, no less than 1 hour, no less than 2 hours, no less than 4 hours, no less than 6 hours, no less than 8 hours, no less than 10 hours, no less than 12 hours, no less than 24 hours, no less than 2 days, no less than 4 days, no less than 6 days, no less than 8) days, no less than 10 days, no less than 12 days, no less than 14 days, no less than 21 days, or no less than 30 days after the first effective amount of a heterodimeric Fc-fused protein.

In certain embodiments, either one of, or both of a heterodimeric Fc-fused protein and/or additional therapeutic agent are administered by a route selected from the group consisting of: intravenous, subcutaneous, cutaneous, oral, intramuscular, and intraperitoneal. In certain embodiments, either one of, or both of a heterodimeric Fc-fused protein and/or additional therapeutic agent are administered by intravenously. In certain embodiments, either one of, or both of, or any combination thereof, a heterodimeric Fc-fused protein and/or additional therapeutic agent are administered orally.

It is understood by the skilled artisan that the unit dose forms of the present disclosure may be administered in the same or different physical forms, i.e. orally via capsules or tablets and/or by liquid via IV infusion, and so on. Moreover, the unit dose forms for each administration may differ by the particular route of administration. Several various dosage forms may exist for either one of, or both of, the combination of a heterodimeric Fc-fused protein and additional therapeutic agents. Because different medical conditions can warrant different routes of administration, the same components of the combination described herein may be exactly alike in composition and physical form and yet may need to be given in differing ways and perhaps at differing times to alleviate the condition. For example, a condition such as persistent nausea, especially with vomiting, can make it difficult to use an oral dosage form, and in such a case, it may be necessary to administer another unit dose form, perhaps even one identical to other dosage forms used previously or afterward, with an inhalation, buccal, sublingual, or suppository route instead or as well. The specific dosage form may be a requirement for certain combinations of a heterodimeric Fc-fused protein and additional therapeutic agents, as there may be issues with various factors like chemical stability or pharmacokinetics.
(i) NK-Targeting Therapy

In certain embodiments, the heterodimeric Fc-fused protein therapy is combined with NK targeting therapies. For example, in an embodiment, the heterodimeric Fc-fused protein is coadministered with a therapeutic agent that targets NKp46. In certain embodiments, the therapeutic agent that targets NKp46 also binds CD16, one or more tumor-associated antigens, or a combination thereof. Exemplary therapeutic agents that target NKp46 are described in more detail in U.S. Application No. US20170198038A1, herein incorporated by reference for all purposes.

In certain embodiments, the heterodimeric Fc-fused protein therapy is combined with bi- and tri-specific killer engagers (BiKEs and TriKEs) therapies, including BiKE and TriKE therapies targeting NK cells. BiKEs and TriKEs are constructed from a single heavy (VH) and light (VL) chain of the variable region of each antibody of interest. VH and VL domains are joined by a short flexible polypeptide linker to prevent dissociation. BiKEs and TriKEs are described in more detail in U.S. Application Nos. US20180282386A1 and US20180258396A1, herein incorporated by reference for all purposes. BiKEs and TriKEs can contain a binding domain specific for an NK cell.

In certain embodiments, BiKE and TriKE therapies are used in combination with the heterodimeric Fc-fused protein therapy to treat subjects known or suspected of having High-risk Myelodysplastic Syndrome, Acute Myelogenous Leukemia, Systemic Mastocytosis, or Mast Cell Leukemia. In certain embodiments, BiKE and TriKE therapies are administered as a single course of 3 weekly treatment blocks. In certain embodiments, a treatment block comprises 4 consecutive 24-hour continuous infusions (approximately 96 hours) followed by a 72 hour break. In certain embodiments, BiKE and TriKE therapies are administered at a dose of 5 μg/kg/day, 10 μg/kg/day, 25 μg/kg/day, 50 μg/kg/day, 100 μg/kg/day, or 200 μg/kg/day. In certain embodiments, BiKE and TriKE therapies are administered at a dose of at least 5 μg/kg/day, at least 10 μg/kg/day, at least 25 μg/kg/day, at least 50 μg/kg/day, at least 100 μg/kg/day, or at least 200 μg/kg/day. In certain embodiments, BiKE and TriKE therapies are administered at a dose of at least 1 μg/kg/day. In certain embodiments, BiKE and TriKE therapies are administered at a dose of at least 5 μg/kg/day. In certain embodiments, BiKE and TriKE therapies are administered at a dose of at least 200 μg/kg/day. In certain embodiments, BiKE and TriKE therapies are administered at a dose of at least 500 μg/kg/day. In certain embodiments, BiKE and TriKE therapies are administered at a dose of at least 1000 μg/kg/day. In certain embodiments, BiKE and TriKE therapies are administered at a dose of 200 μg/kg/day or less. In certain embodiments, BiKE and TriKE therapies are administered at a dose of 500 μg/kg/day or less. In certain embodiments, BiKE and TriKE therapies are administered at a dose of 1000 μg/kg/day or less. In certain embodiments, BiKE and TriKE therapies are administered at a dose of 1-200 μg/kg/day. In certain embodiments, BiKE and TriKE therapies are administered at a dose of 5-200 μg/kg/day. In certain embodiments, BiKE and TriKE therapies are administered at a dose of 1-500 μg/kg/day. In certain embodiments, BiKE and TriKE therapies are administered at a dose of 1-1000 μg/kg/day. In certain embodiments, BiKE and TriKE therapies are administered at a dose of 5-500 μg/kg/day. In certain embodiments, BiKE and TriKE therapies are administered at a dose of 5-1000 μg/kg/day. In certain embodiments, BiKE and TriKE therapies are administered at a maximum-tolerated dose. In certain embodiments, BiKE and TriKE therapies are administered at less than maximum-tolerated dose.
(ii) Multi-specific Binding Protein (“TriNKET”) Therapy

In certain embodiments, the heterodimeric Fc-fused protein therapy is combined with a therapy comprising a multi-specific binding protein, which comprises: (a) a first antigen-binding site that binds NKG2D; (b) a second antigen-binding site that binds a tumor-associated antigen; and (c) an antibody Fc domain or a portion thereof sufficient to bind CD16, or a third antigen-binding site that binds CD16 (“TriNKET”) (for example, multi-specific binding proteins comprising various NKG2D-binders and tumor-associated antigen-binding sites described in international publication no. WO 2019/157332, whose contents relating to the multi-specific binding proteins described therein are incorporated by reference herein), to treat subjects known or suspected of having cancer. Exemplary tumor-associated antigens include, but are not limited to, HER2, CD20, CD33, B-cell maturation antigen (BCMA), EpCAM, CD2, CD19, CD25, CD30, CD38, CD40, CD52, CD70, CLL1/CLEC12A, FLT3, EGFR/ERBB1, IGF1R, HER3/ERBB3, HER4/ERBB4, MUC1, cMET, SLAMF7, PSCA, MICA, MICB, TRAILR1, TRAILR2, MAGE-A3, B7.1, B7.2, CTLA4, HLA-E, and PD-L1.

In certain embodiments, the heterodimeric Fc-fused protein therapy is combined with a therapy comprising a dose of a multi-specific binding protein based on body weight. For example, doses of a multi-specific binding protein based on body weight are from about 0.01 μg to about 100 mg per kg of body weight, such as about 0.01 μg to about 100 mg/kg of body weight, about 0.01 μg to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight.

In certain embodiments, the heterodimeric Fc-fused protein therapy is combined with a therapy comprising doses of a multi-specific binding protein given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of a multi-specific binding protein could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.
(iii) Chimeric antigen receptors (CARs) Therapy

In certain embodiments, the heterodimeric Fc-fused protein therapy is combined with a CAR therapy. The term “chimeric antigen receptor” or alternatively a “CAR” refers to a recombinant polypeptide construct comprising at least an extracellular antigen binding domain, a transmembrane domain and an intracellular signaling domain comprising a functional signaling domain derived from a stimulatory molecule (also referred to herein as a “primary signaling domain”).

Accordingly, in certain embodiments, the CAR comprises an extracellular antigen-binding site that binds tumor-associated antigen, a transmembrane domain, and an intracellular signaling domain comprising a primary signaling domain. In certain embodiments, the CAR further comprises one or more functional signaling domains derived from at least one costimulatory molecule (also referred to as a “costimulatory signaling domain”).

In one embodiment, the CAR comprises a chimeric fusion protein comprising a tumor-associated antigen-binding domain (e.g., tumor-associated antigen-binding scFv domain) comprising a heavy chain variable domain and a light chain variable domain as an extracellular antigen binding domain, a transmembrane domain, and an intracellular signaling domain comprising a primary signaling domain. In one embodiment, the CAR comprises a chimeric fusion protein comprising a tumor-associated antigen-binding domain (e.g., tumor-associated antigen-binding scFv domain) comprising a heavy chain variable domain and a light chain variable domain as an extracellular antigen binding domain, a transmembrane domain and an intracellular signaling domain comprising a costimulatory signaling domain and a primary signaling domain. In certain embodiments, the CAR comprises a chimeric fusion protein comprising a tumor-associated antigen-binding domain (e.g., tumor-associated antigen-binding scFv domain) comprising a heavy chain variable domain and a light chain variable domain as an extracellular antigen binding domain, a transmembrane domain, and an intracellular signaling domain comprising two costimulatory signaling domains and a primary signaling domain. In one embodiment, the CAR comprises a chimeric fusion protein comprising a tumor-associated antigen-binding domain comprising a heavy chain variable domain and a light chain variable domain as an extracellular antigen binding domain, a transmembrane domain, and an intracellular signaling domain comprising at least two costimulatory signaling domains and a primary signaling domain.

With respect to the transmembrane domain, in various embodiments, the CAR is designed to comprise a transmembrane domain that is fused to the extracellular domain of the CAR. In one embodiment, the transmembrane domain is one that naturally is associated with one of the domains in the CAR. In some instances, the transmembrane domain can be selected or modified by amino acid substitution to avoid binding of such domains to the transmembrane domains of the same or different surface membrane proteins to minimize interactions with other members of the receptor complex. In another embodiment, the transmembrane domain is capable of homodimerization with another CAR on the CAR T cell surface. In another embodiment, the amino acid sequence of the transmembrane domain may be modified or substituted so as to minimize interactions with the binding domains of the native binding partner present in the same CAR T cell.

The transmembrane domain may be derived from any naturally occurring membrane-bound or transmembrane protein. In one embodiment, the transmembrane region is capable of signaling to the intracellular domain(s) whenever the CAR has bound to a target. In some embodiments, the transmembrane domain comprises the transmembrane region(s) of one or more proteins selected from the group consisting of TCR α chain, TCR β chain, TCR ζ chain, CD28, CD3ε, CD45, CD4, CD5, CD8, CD9, CD16, CD22, CD33, CD37, CD64, CD80, CD86, CD134, CD137, and CD154. In some embodiments, the transmembrane domain comprises the transmembrane region(s) of one or more protein(s) selected from the group consisting of KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7Rα, ITGA1, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, and NKG2C.

The extracellular tumor-associated antigen-binding domain (e.g., tumor-associated antigen-binding scFv domain) can be connected to the transmembrane domain by a hinge region. A variety of hinges can be employed, including but not limited to the human Ig hinge (e.g., an IgG4 hinge, an IgD hinge), a Gly-Ser linker, a (G₄S)₄ linker, a KIR2DS2 hinge, and a CD8α hinge.

The intracellular signaling domain of the CAR is responsible for activation of at least one of the specialized functions of the immune cell (e.g., cytolytic activity or helper activity, including the secretion of cytokines, of a T cell) in which the CAR has been placed in. Thus, as used herein, the term “intracellular signaling domain” refers to the portion of a protein which transduces an effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain of the CAR comprises a primary signaling domain (i.e. a functional signaling domain derived from a stimulatory molecule) and one or more costimulatory signaling domains (i.e. functional signaling domains derived from at least one costimulatory molecule).

As used herein, the term “stimulatory molecule” refers to a molecule expressed by an immune cell, e.g., a T cell, an NK cell, or a B cell, that provide the cytoplasmic signaling sequence(s) that regulate activation of the immune cell in a stimulatory way for at least some aspect of the immune cell signaling pathway. In one embodiment, the signal is a primary signal that is initiated by, for instance, binding of a TCR/CD3 complex with an MHC molecule loaded with a peptide, and which leads to mediation of a T cell response, including, but not limited to, proliferation, activation, differentiation, and the like.

Primary signaling domains that act in a stimulatory manner may contain signaling motifs which are known as immunoreceptor tyrosine-based activation motifs or ITAMs. Examples of ITAM containing cytoplasmic signaling sequences that are of particular use in the present disclosure include those derived from CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12. In one embodiment, the primary signaling domain in any one or more CARs comprises a cytoplasmic signaling sequence derived from CD3-zeta.

In some embodiments, the primary signaling domain is a functional signaling domain of TCR zeta, FcR gamma, FcR beta, CD3 gamma, CD3 delta, CD3 epsilon, CD5, CD22, CD79a, CD79b, CD66d, 4-1BB, and/or CD3-zeta. In an embodiment, the intracellular signaling domain comprises a functional signaling domain of CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and/or DAP12. In a particular embodiment, the primary signaling domain is a functional signaling domain of the zeta chain associated with the T cell receptor complex.

As used herein, the term “costimulatory molecule” refers to a cognate binding partner on a T cell that specifically binds with a costimulatory ligand, thereby mediating a costimulatory response by the T cell, such as, but not limited to, proliferation. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1, CD11a/CD18), CD2, CD7, CD258 (LIGHT), NKG2C, B7-H3, and a ligand that specifically binds with CD83, and the like. Further examples of such costimulatory molecules include CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11 b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and a ligand that specifically binds with CD83. In some embodiments, the costimulatory signaling domain of the CAR is a functional signaling domain of a costimulatory molecule described herein, e.g., OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof.

As used herein, the term “signaling domain” refers to the functional portion of a protein which acts by transmitting information within the cell to regulate cellular activity via defined signaling pathways by generating second messengers or functioning as effectors by responding to such messengers.

The cytoplasmic signaling sequences within the cytoplasmic signaling portion of the CAR may be linked to each other in a random or specified order. Optionally, a short oligo- or polypeptide linker, for example, between 2 and 10 amino acids in length may form the linkage.
(iv) Antibody Therapy

In certain embodiments, the heterodimeric Fc-fused protein therapy is combined with an antibody therapy to treat subjects known or suspected of having cancer.

In certain embodiments, the heterodimeric Fc-fused protein is combined with a therapy comprising an anti-HER2 binding domain, such as an anti-HER2 antibody or anti-HER2 antibody platforms (e.g., a bi-specific or tri-specific antibody comprising an anti-HER2 binding domain, anti-HER2 antibody-drug conjugates, or anti-HER2 CAR). Anti-HER2 antibodies include, but are not limited to, trastuzumab (HERCEPTIN®—Roche/Genentech; Kanjinti-Amgen), pertuzumab (PERJETA®—Roche/Genentech), and MGAH22 (described in detail in U.S. Pat. No. 8,802,093, herein incorporated by reference for all purposes). Anti-HER2 antibody platforms include, but are not limited to, ertumaxomab (REXOMUN®—Creative Biolabs) and trastuzumab emtansine (ado-trastuzumab emtansine/T-DM1; KADCYLA®—Roche/Genentech). In certain embodiments, the anti-HER2 binding domain therapy is used in combination with the heterodimeric Fc-fused protein therapy to treat subjects known or suspected of having cancer. In certain embodiments, the anti-HER2 binding domain therapy is administered by IV infusion. In certain embodiments, the anti-HER2 binding domain therapy is administered at a dose of 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day. In certain embodiments, the anti-HER2 binding domain therapy is administered at a dose of at least 1 mg/kg/day, at least 2 mg/kg/day, at least 3 mg/kg/day, at least 4 mg/kg/day, at least 5 mg/kg/day, at least 6 mg/kg/day, at least 7 mg/kg/day, at least 8 mg/kg/day, at least 9 mg/kg/day, at least 10 mg/kg/day. In certain embodiments, the anti-HER2 binding domain therapy is administered at a dose of less than 1 mg/kg/day.

In certain embodiments, the anti-HER2 binding domain therapy is used in combination with the heterodimeric Fc-fused protein therapy to treat subjects known or suspected of having breast cancer, e.g., a subject diagnosed with metastatic HER2-overexpressing breast cancer. In certain embodiments, the anti-HER2 binding domain therapy is administered at 4 mg/kg/day. In certain embodiments, the anti-HER2 binding domain therapy is administered at 4 mg/kg/day by IV infusion over 90 minutes. In certain embodiments, the anti-HER2 binding domain therapy is administered at 2 mg/kg/day. In certain embodiments, the anti-HER2 binding domain therapy is administered at 2 mg/kg/day by IV infusion over 30 minutes. In certain embodiments, the anti-HER2 binding domain therapy is administered at an initial dose of 4 mg/kg/day, then subsequently administered weekly at 2 mg/kg/day. In certain embodiments, the anti-HER2 binding domain therapy is administered at an initial dose of 4 mg/kg/day, then subsequently administered weekly at 2 mg/kg/day for 52 weeks.

In certain embodiments, the anti-HER2 binding domain therapy is used in combination with the heterodimeric Fc-fused protein therapy to treat subjects known or suspected of having gastric cancer, e.g., a subject diagnosed with metastatic HER2-overexpressing gastric cancer. In certain embodiments, the anti-HER2 binding domain therapy is administered at 8 mg/kg/day. In certain embodiments, the anti-HER2 binding domain therapy is administered at 8 mg/kg/day by IV infusion over 90 minutes. In certain embodiments, the anti-HER2 binding domain therapy is administered at 6 mg/kg/day. In certain embodiments, the anti-HER2 binding domain therapy is administered at 6 mg/kg/day by IV infusion over 30-90 minutes. In certain embodiments, the anti-HER2 binding domain therapy is administered at an initial dose of 8 mg/kg/day, then subsequently administered weekly at 6 mg/kg/day. In certain embodiments, the anti-HER2 binding domain therapy is administered at an initial dose of 8 mg/kg/day, then subsequently administered weekly at 6 mg/kg/day for 52 weeks.

In certain embodiments, the heterodimeric Fc-fused protein therapy is combined with a therapy comprising an anti-CD20 binding domain, such as an anti-CD20 antibody or anti-CD20 antibody platforms (e.g., a bi-specific or tri-specific antibody comprising an anti-CD20 binding domain, anti-CD20 antibody-drug conjugates, or anti-CD20 CAR). Anti-CD20 antibodies include, but are not limited to, rituximab (RITUXAN®-Roche/Genentech), ocrelizumab (OCREVUS®-Roche/Genentech), obinutuzumab (GAZYVA®-Roche/Genentech), ofatumumab (ARZERRA® Novartis), and veltuzumab. In certain embodiments, the anti-CD20 binding domain therapy is used in combination with the heterodimeric Fc-fused protein therapy to treat subjects known or suspected of having cancer. In certain embodiments, the anti-CD20 binding domain therapy is administered by IV infusion. In certain embodiments, the anti-CD20 binding domain therapy is administered at a dose of 100 mg/m², 200 mg/m², 300 mg/m², 400 mg/m², 500 mg/m², 600 mg/m², 700 mg/m², 800 mg/m², 900 mg/m², or 1000 mg/m². In certain embodiments, the anti-CD20 binding domain therapy is administered at a dose of 375 mg/m². In certain embodiments, the anti-CD20 binding domain therapy is administered at a dose of at least 100 mg/m², at least 200 mg/m², at least 300 mg/m², at least 400 mg/m², at least 500 mg/m², at least 600 mg/m², at least 700 mg/m², at least 800 mg/m², at least 900 mg/m², or at least 1000 mg/m². In certain embodiments, the anti-CD20 binding domain therapy is administered at a dose of less than 400 mg/m². In certain embodiments, the anti-CD20 binding domain therapy is administered at a dose of less than 375 mg/m2.

In certain embodiments, the anti-CD20 binding domain therapy is used in combination with the heterodimeric Fc-fused protein therapy to treat subjects known or suspected of having Non-Hodgkin's Lymphoma (NHIL). In certain embodiments, the anti-CD20 binding domain therapy is administered at a dose of 375 mg/m² by IV-infusion. In certain embodiments, the anti-CD20 binding domain therapy is administered at a dose less than 375 mg/m² by IV-infusion.

In certain embodiments, the anti-CD20 binding domain therapy is used in combination with the heterodimeric Fc-fused protein therapy to treat subjects known or suspected of having Chronic Lymphocytic Leukemia (CLL). In certain embodiments, the anti-CD20 binding domain therapy is administered at a dose of 375 mg/m² by IV-infusion in a first cycle, and at a dose of 500 mg/m² by IV-infusion per cycle in an additional 2-6 cycles. In certain embodiments, the anti-CD20 binding domain therapy is administered at a dose less than 375 mg/m² by IV-infusion. The combined anti-CD20 binding domain and heterodimeric Fc-fused protein therapy can be used in combination with fludarabine and cyclophosphamide (FC).

In certain embodiments, the anti-CD20 binding domain therapy is used in combination with the heterodimeric Fc-fused protein therapy to treat subjects known or suspected of having Rheumatoid Arthritis (RA). In certain embodiments, the anti-CD20 binding domain therapy is administered as two doses of 1000 mg, doses separated 2 weeks, by IV-infusion. In certain embodiments, the anti-CD20 binding domain therapy is administered as two doses of 1000 mg, doses separated 2 weeks, by IV-infusion up to 24 weeks. In certain embodiments, the combined anti-CD20 binding domain and heterodimeric Fc-fused protein therapy is coadministered with methotrexate.

In certain embodiments, the heterodimeric Fc-fused protein therapy is combined with a therapy comprising an antibody therapy comprising an agonist antibody. In certain embodiments, the agonist antibody is an anti-4-1BB antibody, an anti-CD137 antibody, an anti-FAP antibody, an anti-OX40 antibody, an anti-CD40 antibody, an anti-GITR antibody, or an anti-CD27 antibody. In certain embodiments, the agonist antibody is a bispecific antibody. In certain embodiments, the agonist antibody is a multispecific antibody, e.g., a bispecific antibody, comprising two or more antigen binding domains selected from an anti-4-1BB antibody, an anti-CD137 antibody, an anti-FAP antibody, an anti-OX40 antibody, an anti-CD40 antibody, an anti-GITR antibody, or an anti-CD27 antibody. An illustrative example is a bispecific agonist antibody targeting 4-1BB and CD137, such as utomilumab (Pfizer).
(v) Checkpoint Inhibitor Therapy

In certain embodiments, the heterodimeric Fc-fused protein therapy can be combined with a checkpoint inhibitor therapy. Illustrative immune checkpoint molecules that can be targeted for blocking or inhibition include, but are not limited to, CTLA-4, 4-1BB (CD137), 4-1BBL (CD137L), PDL1, PDL2, PD1, B7-H3, B7-H4, BTLA, HVEM, TIM3, GAL9, LAG3, TIM3, B7H3, B7H4, VISTA, KIR, 2B4 (belongs to the CD2 family of molecules and is expressed on all NK, γδ, and memory CD8+ (αβ) T cells), CD160 (also referred to as BY55), and CGEN-15049. Immune checkpoint inhibitors include antibodies, or antigen binding fragments thereof, or other binding proteins, that bind to and block or inhibit the activity of one or more of CTLA-4, PDL1, PDL2, PD1, B7-H3, B7-H4, BTLA, HVEM, TIM3, GAL9, LAG3, TIM3, B7H3, B7H4, VISTA, KIR, 2B4, CD160, and CGEN-15049. Illustrative immune checkpoint inhibitors include nivolumamb (anti-PD-1; OPDIVO®-BMS), AMP224 (anti-PD-1; NCI), pembrolizumab (anti-PD-1; MK-3475/KEYTRUDA®-Merck), pidilizumab (anti-PD-1 antibody; CT-011-Teva/CureTech), atezolizumab (anti-PD-L1; TECENTRIQ®-Roche/Genentech), durvalumab (anti-PD-L1; MEDI4736/IMFINZI®-Medimmune/AstraZeneca), avelumab (anti-PD-L1; BAVENCIO®-Pfizer), BMS-936559 (anti-PD-L1-BMS), ipilimumab (anti-CTLA-4; YERVOY®-BMS), tremelimumab (anti-CTLA-4; Medimmune/AstraZeneca), lirilumab (anti-KIR; BMS), monalizumab (anti-NKG2A; Innate Pharma/AstraZeneca), BY55 (anti-CD160), anti-OX40. anti-TIM3, and anti-LAG3.

In certain embodiments, the method of the present invention further comprises administering to the subject an anti-PD-1 antibody. Many anti-PD-1 antibodies have been developed for therapeutic purposes and are described in, for example, Gong et al., (2018) J. ImmunoTher Cancer (2018) 6:8. In certain embodiments, the anti-PD-1 antibody is pembrolizumab. In certain embodiments pembrolizumab can be administered via various routes, e.g., intravenously, subcutaneously, intramuscularly, or intraperitoneally. In certain embodiments, pembrolizumab is administered intravenously. In certain embodiments, pembrolizumab can be administered at a dose of about 100 mg, about 125 mg, about 150 mg, about 175 mg, about 200 mg, about 225 mg, about 250 mg, about 275 mg, about 300 mg, or about 400 mg. In certain embodiments, pembrolizumab is administered at a dose of about 200 mg every 3 weeks. In certain embodiments, pembrolizumab is administered at a dose of about 400 mg every 6 weeks. In certain embodiments about 200 mg of pembrolizumab is administered on Day 1 of the initial treatment cycle. In certain embodiments, if the subject receives one or more subsequent treatment cycles, 200 mg of pembrolizumab is administered once every three weeks in the subsequent treatment cycles, starting from Day 1 of the first subsequent treatment cycle. In some embodiments, administration of pembrolizumab can precede each administration of the pharmaceutical formulation, can be concurrent with each administration of the pharmaceutical formulation, or follow each administration of the pharmaceutical formulation. In certain embodiments, administration of pembrolizumab precedes each administration of the pharmaceutical formulation. In some embodiments, the pharmaceutical formulation can be administered within about 10 minutes, about 20 minutes, about 30 minutes, about 40 minutes, about 50 minutes, about 1 hour, about 1.5 hours, about 2 hours, about 2.5 hours, about 3 hours, about 3.5 hours, about 4 hours, about 4.5 hours, or about 5 hours after completion of administration of pembrolizumab. In certain embodiments, the pharmaceutical formulation is administered within 1 hour after completion of administration of pembrolizumab.

In some embodiments, the pharmaceutical formulation administered in combination with pembrolizumab is for the treatment of a cancer selected from the group consisting of: melanoma, non-small cell lung cancer (NSCLC), small cell lung cancer (SCLC), head and neck squamous cell carcinoma (HNSCC), classical Hodgkin lymphoma, primary mediastinal large B-cell lymphoma, urothelial carcinoma, microsatellite instability-high cancer, gastric cancer, oesophageal cancer, cervical cancer, hepatocellular carcinoma, Merkel cell carcinoma, renal cell carcinoma, and endometrial carcinoma.

In certain embodiments, the anti-PD-1 antibody is nivolumab. In certain embodiments nivolumab can be administered via various routes, e.g., intravenously, subcutaneously, intramuscularly, or intraperitoneally. In certain embodiments, nivolumab is administered intravenously. In some embodiments, nivolmab can be administered at a dose of about 200 mg, about 220 mg, about 240 mg, about 260 mg about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, or about 600 mg. In certain embodiments, nivolumab is administered at a dose of about 240 mg. In certain embodiments, nivolumab is administered at a dose of about 240 mg once about every two weeks. In certain embodiments, nivolumab is administered at a dose of about 360 mg. In certain embodiments, nivolumab is administered at a dose of about 360 mg once about every three weeks. In certain embodiments, nivolumab is administered at a dose of about 480 mg. In certain embodiments, nivolumab is administered at a dose of about 480 mg once about every four weeks. In some embodiments, administration of nivolumab can precede each administration of the pharmaceutical formulation, can be concurrent with each administration of the pharmaceutical formulation, or follow each administration of the pharmaceutical formulation. In certain embodiments, administration of nivolumab precedes each administration of the pharmaceutical formulation. In some embodiments, the pharmaceutical formulation can be administered within about 10 minutes, about 20 minutes, about 30 minutes, about 40 minutes, about 50 minutes, about 1 hour, about 1.5 hours, about 2 hours, about 2.5 hours, about 3 hours, about 3.5 hours, about 4 hours, about 4.5 hours, or about 5 hours after completion of administration of nivolumab. In certain embodiments, the pharmaceutical formulation is administered within 1 hour after completion of administration of nivolumab. In some embodiments, the pharmaceutical formulation administered in combination with nivolumab is for the treatment of a cancer selected from the group consisting of: melanoma, non-small cell lung cancer (NSCLC), small cell lung cancer (SCLC), renal cell carcinoma, classical Hodgkin lymphoma, head and neck squamous cell carcinoma (HNSCC), colorectal cancer, hepatocellular carcinoma, bladder cancer, and oesophageal cancer. In certain embodiments the cancer is melanoma. In certain embodiments the melanoma is unresectable or metastatic. In some embodiments the cancer is colorectal cancer. In certain embodiments, the colorectal cancer is microsatellite instability-high (MSI-H) or mismatch repair deficient (dMMR) metastatic colorectal cancer.

In certain embodiments, the checkpoint inhibitor therapy is used in combination with the heterodimeric Fc-fused protein therapy to treat subjects known or suspected of having cancer. In certain embodiments, the checkpoint inhibitor therapy is administered by IV infusion. In certain embodiments, the checkpoint inhibitor therapy is administered by IV infusion over 30 minutes. In certain embodiments, the checkpoint inhibitor therapy is administered every 3 weeks. In certain embodiments, the checkpoint inhibitor therapy is administered at a dose of about 100 mg, about 200 mg, about 300 mg, about 400 mg, about 500 mg, about 600 mg, about 700 mg, about 800 mg, about 900 mg, or about 1000 mg. In certain embodiments, the checkpoint inhibitor therapy is administered at a dose of 200 mg. In certain embodiments, the checkpoint inhibitor therapy is administered at a dose of at least 100 mg, at least 200 mg, at least 300 mg, at least 400 mg, at least 500 mg, at least 600 mg, at least 700 mg, at least 800 mg, at least 900 mg, or at least 1000 mg. In certain embodiments, the checkpoint inhibitor therapy is administered at a dose of less than 200 mg. In certain embodiments, the checkpoint inhibitor therapy is used in combination with the heterodimeric Fc-fused protein therapy to treat subjects known or suspected of having melanoma, non-small cell lung cancer (NSCLC), head and neck squamous cell carcinoma (HNSCC), classical Hodgkin lymphoma, primary mediastinal large B-Cell lymphoma, bladder cancer, urothelial carcinoma, microsatellite instability-high cancer, colorectal cancer, gastric cancer, oesophageal cancer, cervical cancer, hepatocellular carcinoma, Merkel cell carcinoma, renal cell carcinoma (RCC), endometrial carcinoma, cutaneous T cell lymphoma, and triple negative breast cancer.
(vi) Additional Cytokine Therapy

In some embodiments, the heterodimeric Fc-fused protein therapy is combined with one or more additional cytokine therapies, one or more chemokine therapies, or combinations thereof. In some embodiments, the heterodimeric Fc-fused protein therapy is combined with one or more additional cytokine therapies. In some embodiments, the heterodimeric Fc-fused protein therapy is combined with one or more chemokine therapies. In some embodiments, the cytokine therapy comprises a pro-inflammatory cytokine, a Th1 cytokine, or a Th2 cytokine. In some embodiments, the cytokine therapy comprises a recombinant human cytokine or chemokine.

In some embodiments, the cytokine therapy includes a cytokine that is an interleukin (e.g., IL-1, IL-2, IL-6, IL-7, IL-8, IL-12, IL-13, IL-15, IL-16, IL-18, IL-21 and IL-22). In some embodiments, the cytokine therapy includes a cytokine that is growth factor (e.g., tumor necrosis factor (TNF), LT, EMAP-II, GM-CSF, FGF and PDGF). In some embodiments, the cytokine therapy comprises an anti-inflammatory cytokine (e.g., IL-4, IL-10, IL-11, IL-13 and TGF).

In some embodiments, the chemokine therapy includes a pro-inflammatory chemokine (e.g., GRO-α, GRO-b, LIX, GCP-2, MIG, IP10, I-TAC, and MCP-1, RANTES, Eotaxin, SDF-1, and MIP3a). In some embodiments, the chemokine therapy includes a chemokine receptor. In some embodiments, the chemokine therapy includes a CXC chemokine receptor (e.g., CXCR1, CXCR2, CXCR3, CXCR4, CXCR5, CXCR6, and CXCR7), a CC chemokine receptor (e.g., CCR1, CCR2, CCR3, CCR4, CCR5, CCR6, CCR7, CCR8, CCR9, CCR10, and CCR11), a CX3C chemokine receptor (e.g., CX3C11), or a XC chemokine receptor (e.g., XCR1). In some embodiments, the chemokine therapy comprises a G protein-linked transmembrane receptor.

In some embodiments, the cytokine therapy comprises a cytokine therapy that synergizes with the IL-12 signaling. In some embodiments, the cytokine therapy comprises an IL-2 cytokine or a derivative thereof. In some embodiments, the IL-2 therapy is aldesleukin (Proleukin-Prometheus Therapeutics). In some embodiments, the IL-2 therapy and/or aldesleukin is administered intravenously. In some embodiments, the cytokine therapy comprises an IL-15 cytokine or a derivative thereof. In some embodiments, the IL-15 therapy is ALT-803 (Altor Bioscience) or NKTR-255 (Nektar). In some embodiments, the IL-15 therapy, NKTR-255, and/or ALT-803 is administered subcutaneously. In some embodiments, the chemokine therapy comprises a CXCL9 chemokine, a CXCL10 chemokine, or derivatives thereof.

In some embodiments, the cytokine or chemokine therapy includes administering a cytokine or chemokine to a subject. In some embodiments, the cytokine or chemokine therapy includes administering a recombinant cytokine or chemokine to a subject. In some embodiments, the cytokine or chemokine therapy includes engineering a cell to produce the cytokine or chemokine. In some embodiments, the cytokine or chemokine therapy includes engineering a cell ex vivo, in vitro, or in vivo to produce the cytokine or chemokine.

In some embodiments, the cytokine or chemokine therapy includes engineering a cell to produce the cytokine or chemokine using a viral vector-based delivery platform such as vaccinia, fowlpox, self-replicating alphavirus, marabavirus, adenovirus (See, e.g., Tatsis et al., Adenoviruses, Molecular Therapy (2004) 10, 616-629), a lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In vivo Gene Delivery, J. Virol. (1998) 72 (12): 9873-9880), or an adeno-associated virus (“AAV”) vector, as described in more detail in U.S. Pat. No. 5,173,414; Tratschin et al, Mol. Cell. Biol. 5:3251-3260 (1985); Tratschin, et al, Mol. Cell, Biol. 4:2072-2081 (1984); Hermonat et al., PNAS 81:64666470 (1984); and Samulski et al, J. Virol. 63:03822-3828 (1989)). In some embodiments, the cytokine or chemokine therapy includes engineering a cell to produce the cytokine or chemokine using a LNP, liposome, or an exosome. In some embodiments, the cytokine or chemokine therapy includes engineering a cell to produce the cytokine or chemokine using genome editing, such as using a nuclease-based genome editing systems (e.g., a Clustered Regularly Interspaced Short Palindromic Repeats (CRISPR) family, a Transcription activator-like effector nuclease (TALEN), a zinc-finger nuclease (ZFN), and a homing endonuclease (HE) based genome editing system or a derivative thereof). In some embodiments, the cytokine or chemokine therapy includes engineering a cell to produce the cytokine or chemokine using electroporation.
(vii) Innate Immune System Agonist Therapy

In some embodiments, the heterodimeric Fc-fused protein therapy is combined with one or more innate immune system agonists.

In some embodiments, the innate immune system agonist comprises a toll-like receptor (TLR) agonist. In some embodiments, the TLR agonist comprises a TLR1/2, TLR2/6, TLR3, TLR4, TLR7, TLR8, TLR7/8, or TLR9 agonist. In some embodiments, a TLR2/6 agonist comprises lipoproteins, such as bacterial lipoproteins or derivatives, such as Pam2CSK4. In some embodiments, a TLR1/2 agonist comprises lipoproteins. In some embodiments, a TLR3 agonist comprises a dsRNA analog, such as rintatolimod (AMPLIGEN®-Hemispherx Biopharma) or poly IC-LC (e.g., HILTONOL®). In some embodiments, a TLR4 agonist comprises lipopolysaccharide (LPS, also referred to as endotoxin) or derivatives, such as lipid A. In some embodiments, a TLR7 agonist comprises a ssRNA or derivatives or imidazoquinoline derivatives including, but not limited to, resiquimod (also referred to as R848), imiquimod (ZYCLARA®, Aldara-Medicis), and gardiquimod. In some embodiments, a TLR7 agonist is also a TLR8 agonist, such as imiquimod or Medi-9197 (AstraZeneca/MedImmune). In some embodiments, a TLR9 agonist comprises a CpG-containing oligodeoxynucleotide (CpG-ODN) or SD-101 (Dynavax).

In some embodiments, the innate immune system agonist comprises a Stimulator of interferon genes (STING) agonist. In some embodiments, the STING agonist comprises a cyclic-di-nucleotide (CDN). Is some embodiments, the CDN comprises a cyclic-di-AMP, a cyclic-di-GMP, or a cyclic-GMP-AMP (cGAMP). In some embodiments, the STING agonist comprises a nucleic acid (e.g., DNA or RNA) that stimulates cGAS. In some embodiments, the STING agonist is ADU-S100 (also referred to as MIW815-Aduro/Novartis).
(viii) Chemotherapy

In certain embodiments, the heterodimeric Fc-fused protein therapy is combined with one or more chemotherapies. In certain embodiments, the heterodimeric Fc-fused protein therapy is combined with one or more chemotherapies to treat a subject diagnosed with cancer. Examples of chemotherapy agents include aldesleukin, alvocidib, antineoplaston AS2-1, antineoplaston A10, anti-thymocyte globulin, amifostine trihydrate, aminocamptothecin, arsenic trioxide, beta alethine, Bcl-2 family protein inhibitor ABT-263, ABT-199, BMS-345541, bortezomib (VELCADE®), bryostatin 1, busulfan, carboplatin, campath-1H, CC-5103, carmustine, caspofungin acetate, clofarabine, cisplatin, Cladribine (LEUSTARIN®), Chlorambucil (LEUKERAN®), Curcumin, cyclosporine, Cyclophosphamide (Cyloxan, Endoxan, Endoxana, Cyclostin), cytarabine, denileukin diftitox, dexamethasone, DT PACE, docetaxel, dolastatin 10, Doxorubicin (ADRIAMYCIN®, ADRIBLASTINE®), doxorubicin hydrochloride, enzastaurin, epoetin alfa, etoposide, Everolimus (RAD001), fenretinide, filgrastim, melphalan, mesna, Flavopiridol, Fludarabine (FLUDARA®), Geldanamycin (17-AAG), ifosfamide, irinotecan hydrochloride, ixabepilone, Lenalidomide (REVLIMID®, CC-5013), lymphokine-activated killer cells, melphalan, methotrexate, mitoxantrone hydrochloride, motexafin gadolinium, mycophenolate mofetil, nelarabine, oblimersen (GENASENSE®) Obatoclax (GX15-070), oblimersen, octreotide acetate, omega-3 fatty acids, oxaliplatin, paclitaxel, PD0332991, PEGylated liposomal doxorubicin hydrochloride, pegfilgrastim, Pentstatin (NIPENT®), perifosine, Prednisolone, Prednisone, R-roscovitine (SELICILIB®, CYC202), recombinant interferon alfa, recombinant interleukin-12, recombinant interleukin-11, recombinant flt3 ligand, recombinant human thrombopoietin, rituximab, sargramostim, sildenafil citrate, simvastatin, sirolimus, Styryl sulphones, tacrolimus, tanespimycin, Temsirolimus (CC1-779), Thalidomide, therapeutic allogeneic lymphocytes, thiotepa, tipifarnib, VELCADE® (BORTEZOMIB® or PS-341), Vincristine (ONCOVIN®), vincristine sulfate, vinorelbine ditartrate, Vorinostat (SAHA), and FR (fludarabine, rituximab), CHOP (cyclophosphamide, doxorubicin, vincristine, prednisone), CVP (cyclophosphamide, vincristine and prednisone), FCM (fludarabine, cyclophosphamide, mitoxantrone), FCR (fludarabine, cyclophosphamide, rituximab), hyperCVAD (hyperfractionated cyclophosphamide, vincristine, doxorubicin, dexamethasone, methotrexate, cytarabine), ICE (iphosphamide, carboplatin and etoposide), MCP (mitoxantrone, chlorambucil, and prednisolone), R-CHOP (rituximab plus CHOP), R-CVP (rituximab plus CVP), R-FCM (rituximab plus FCM), R-ICE (rituximab-ICE), and R-MCP (R-MCP).

In certain embodiments, the heterodimeric Fc-fused protein therapy is combined with one or more chemotherapies to treat a subject diagnosed with colon cancer, rectal cancer, or colorectal cancer (CRC). In certain embodiments, the chemotherapy comprises FOLFOX (5-FU, leucovorin, and oxaliplatin/Eloxatin), FOLFIRI (leucovorin, 5-FU, and irinotecan/Camptosar), FOLFOXIRI (leucovorin, 5-FU, oxaliplatin, and irinotecan), CapeOx (capecitabine and oxaliplatin), 5-FU coadministered with leucovorin, capecitabine (XELODA®) alone, or Trifluridine and tipiracil (LONSURF®). In certain embodiments, the chemotherapy comprises a VEGF targeting agent, such as bevacizumab (AVASTIN®), ziv-aflibercept (ZALTRAP®), ramucirumab (CYRAMZA®), or Regorafenib (STIVARGA®), or an EGFR targeting agent such as cetuximab (ERBITUX) or panitumumab (VECTIBIX®). In certain embodiments, the chemotherapy coadministers a chemotherapy selected from FOLFOX, FOLFIRI, FOLFOXIRI, CapeOx, 5-FU coadministered with leucovorin, capecitabine alone, and Trifluridine/tipiracil together with a VEGF targeting agent or an EGFR targeting agent.

In certain embodiments, the heterodimeric Fc-fused protein therapy is combined with one or more chemotherapies to treat a subject diagnosed with breast cancer. In certain embodiments, the chemotherapy comprises doxorubicin (ADRIAMYCIN®), pegylated liposomal doxorubicin, epirubicin (ELLENCE®), paclitaxel (Taxol), docetaxel (TAXOTERE®), albumin-bound paclitaxel (ABRAXANE®), 5-fluorouracil (5-FU), cyclophosphamide (CYTOXAN®), carboplatin (PARAPLATIN®), cisplatin, vinorelbine (NAVELBINE®), capecitabine (XELODA), gemcitabine (GEMZAR®), ixabepilone (IXEMPRA®), or eribulin (HALAVEN). In certain embodiments, the chemotherapy comprises a combination of two or more chemotherapies selected from doxorubicin (ADRIAMYCIN®), pegylated liposomal doxorubicin, epirubicin (ELLENCE®), paclitaxel (Taxol), docetaxel (TAXOTERE®), albumin-bound paclitaxel (ABRAXANE®), 5-fluorouracil (5-FU), cyclophosphamide (CYTOXAN®), carboplatin (PARAPLATIN®), cisplatin, vinorelbine (NAVELBINE®), capecitabine (XELODA®), gemcitabine (GEMZAR®), ixabepilone (IXEMPRA®), and eribulin (HALAVEN®).

In certain embodiments, the heterodimeric Fc-fused protein therapy is combined with one or more chemotherapies to treat a subject diagnosed with melanoma/skin-cancer. In certain embodiments, the chemotherapy comprises dacarbazine (also called DTIC), temozolomide, nab-paclitaxel, paclitaxel, cisplatin, carboplatin, or vinblastine.
(ix) Targeted Agent Therapy

In certain embodiments, the heterodimeric Fc-fused protein therapy is combined with one or more targeted agents. In general, targeted agents act on specific molecular targets, such as targets associated with cancer. Targeted agents are differentiated from standard chemotherapies in that standard chemotherapies act on all rapidly dividing normal and cancerous cells. Targeted agents include, but are not limited to, a hormone therapy, a signal transduction inhibitor, a gene expression modulator, an apoptosis inducer, an angiogenesis inhibitor, an immunotherapy, a toxin delivery molecule (e.g., an antibody drug-conjugate), and a kinase inhibitor. In certain embodiments, a targeted agent comprises a receptor agonist or ligand.

In certain embodiments, the heterodimeric Fc-fused protein therapy is combined with one or more targeted agents to treat a subject diagnosed with colon cancer, rectal cancer, or colorectal cancer (CRC). In certain embodiments, the targeted agent comprises cetuximab (ERBITUX®), panitumumab (VECTIBIX®), bevacizumab (AVASTIN®), ziv-aflibercept (ZALTRAP®), regorafenib (STIVARGA®), ramucirumab (CYRAMZA®), nivolumab (OPDIVO®), or ipilimumab (YERVOY®).

In certain embodiments, the heterodimeric Fc-fused protein therapy is combined with one or more targeted agents to treat a subject diagnosed with breast cancer. In certain embodiments, the targeted agent comprises everolimus (AFINITOR®), tamoxifen (NOLVADEX®), toremifene (FARESTON®), trastuzumab (HERCEPTIN®), fulvestrant (FASLODEX®), anastrozole (ARIMIDEX®), exemestane (AROMASIN®), lapatinib (TYKERB®), letrozole (FEMARA®), pertuzumab (PERJETA®), ado-trastuzumab emtansine (KADCYLA®), palbociclib (IBRANCE®), ribociclib (KISQALI®), neratinib maleate (NERLYNX™), abemaciclib (VERZENIO™), olaparib (LYNPARZA™), atezolizumab (TECENTRIQ®), or alpelisib (PIQRAY®).

In certain embodiments, the heterodimeric Fc-fused protein therapy is combined with one or more targeted agents to treat a subject diagnosed with melanoma/skin-cancer. In certain embodiments, the targeted agent comprises Vismodegib (ERIVEDGE®), sonidegib (ODOMZO®), ipilimumab (YERVOY®), vemurafenib (ZELBORAF®), trametinib (MEKINIST®), dabrafenib (TAFINLAR®), pembrolizumab (KEYTRUDA®), nivolumab (OPDIVO®), cobimetinib (COTELLIC™), alitretinoin (PANRETIN®), avelumab (BAVENCIO®), encorafenib (BRAFTOVI™), binimetinib (MEKTOVI®), or cemiplimab-rwlc (LIBTAYO®).

In certain embodiments, the heterodimeric Fc-fused protein therapy is combined with a receptor agonist or ligand therapy. In certain embodiments, the receptor agonist or ligand therapy comprises an agonist antibody. In certain embodiments, the receptor agonist or ligand therapy comprises a receptor ligand, such as 4-1BBL or CD40L.
(x) Radiotherapy

In some embodiments, the heterodimeric Fc-fused protein therapy is combined with radiotherapy. In certain embodiments, the heterodimeric Fc-fused protein therapy is combined with a radioisotope particle, such as indium In-111, yttrium Y-90, or iodine I-131. Examples of combination therapies include, but are not limited to, Iodine-131 tositumomab (BEXXAR®), Yttrium-90 ibritumomab tiuxetan (ZEVALIN®), and BEXXAR® with CHOP. In certain embodiments, the radiotherapy comprises external-beam radiation therapy (EBRT), internal radiation therapy (brachytherapy), endocavitary radiation therapy, interstitial brachytherapy, radioembolization, hypofractionated radiation therapy, intraoperative radiation therapy (IORT), 3D-conformal radiotherapy, stereotactic radiosurgery (SRS), or stereotactic body radiation therapy (SBRT).

In certain embodiments, the heterodimeric Fc-fused protein therapy is combined with one or more radiotherapies to treat a subject diagnosed with colon cancer, rectal cancer, or colorectal cancer (CRC). In certain embodiments, the radiotherapy comprises external-beam radiation therapy (EBRT), internal radiation therapy (brachytherapy), endocavitary radiation therapy, interstitial brachytherapy, or radioembolization.

In certain embodiments, the heterodimeric Fc-fused protein therapy is combined with one or more radiotherapies to treat a subject diagnosed with breast cancer. In certain embodiments, the radiotherapy comprises external-beam radiation therapy, hypofractionated radiation therapy, intraoperative radiation therapy (IORT), or 3D-conformal radiotherapy.

In certain embodiments, the heterodimeric Fc-fused protein therapy is combined with one or more radiotherapies to treat a subject diagnosed with melanoma/skin-cancer. In certain embodiments, the radiotherapy comprises stereotactic radiosurgery (SRS; e.g., using a Gamma Knife or linear accelerator) or stereotactic body radiation therapy (SBRT).
(xi) Vaccine and Oncolytic Viruses Therapy

In some embodiments, the heterodimeric Fc-fused protein therapy is combined with one or more immunogenic compositions, e.g., a vaccine composition or an oncolytic virus, capable of raising a specific immune response, e.g., a tumor-specific immune response.

In some embodiments, the heterodimeric Fc-fused protein therapy is combined with a vaccine composition. Vaccine compositions typically comprise a plurality of antigens and or neoantigens specific for the tumor to be targeted. Vaccine compositions can also be referred to as vaccines.

In some embodiments, a vaccine composition further comprises an adjuvant and/or a carrier. In some embodiments, a vaccine composition associates with a carrier such as a protein or an antigen-presenting cell such as a dendritic cell (DC) capable of presenting the peptide to a T-cell. In some embodiments, carriers are scaffold structures, for example a polypeptide or a polysaccharide, to which an antigen or neoantigen, is capable of being associated.

In general, adjuvants are any substance whose admixture into a vaccine composition increases or otherwise modifies the immune response to an antigen or neoantigen. Optionally, adjuvants are conjugated covalently or non-covalently. The ability of an adjuvant to increase an immune response to an antigen is typically manifested by a significant or substantial increase in an immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th response into a primarily cellular, or Th response. Suitable adjuvants include, but are not limited to 1018 ISS, alum, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11). Cytokines can also be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418). In some embodiments, an adjuvant comprises a CpG immunostimulatory oligonucleotide. In some embodiments, an adjuvant comprises a TLR agonist.

Other examples of useful adjuvants include, but are not limited to, chemically modified CpGs (e.g. CpR, Idera), Poly(I:C)(e.g. polyi:CI2U), non-CpG bacterial DNA or RNA as well as immunoactive small molecules and antibodies such as cyclophosphamide, sunitinib, bevacizumab, celecoxib, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives can readily be determined by the skilled artisan without undue experimentation. Additional adjuvants include colony-stimulating factors, such as Granulocyte Macrophage Colony Stimulating Factor (GM-CSF, sargramostim).

In some embodiments, a vaccine composition comprises more than one different adjuvant. In some embodiments, a vaccine composition comprises any adjuvant substance including any of the above or combinations thereof. It is also contemplated that a vaccine and an adjuvant can be administered together or separately in any appropriate sequence.

In some embodiments, a carrier (or excipient) is present independently of an adjuvant. In some embodiments, the function of a carrier is to increase the molecular weight, increase activity or immunogenicity, to confer stability, to increase the biological activity, or to increase serum half-life. In some embodiments, a carrier aids presenting peptides to T-cells. In some embodiments, a carrier comprises any suitable carrier known to the person skilled in the art, for example a protein or an antigen presenting cell. Examples of carrier proteins include, but are not limited to, keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. However, tetanus toxoid and/or diptheria toxoid are suitable carriers. Alternatively, the carrier can be a dextran, for example Sepharose.

In some embodiments, a vaccine comprises a viral vector-based vaccine platform, such as vaccinia, fowlpox, self-replicating alphavirus, maraba virus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In vivo Gene Delivery, J. Virol. (1998) 72 (12): 9873-9880). In general, upon introduction into a host, infected cells express the antigen or neoantigen and thereby elicits a host immune (e.g., CTL) response against the peptide(s).

Dependent on the packaging capacity of the above mentioned viral vector-based vaccine platforms, in some embodiments, the vaccine composition comprises one or more viral-vectors. In some embodiments, viral-vectors comprise sequences flanked by non-mutated sequences, separated by linkers, or preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20(13):3401-10). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of neoantigens, e.g., Salmonella typhi vectors, and the like will be apparent to those skilled in the art from the description herein.

In some embodiments, the heterodimeric Fc-fused protein therapy is combined with an oncolytic virus therapy. In general, an oncolytic virus is a virus engineered to infect and kill mainly cancer cells. In some embodiments, in addition to an oncolytic virus killing a cancer cell, the oncolytic virus induces an immune response to the cancer cell.

In certain embodiments, the heterodimeric Fc-fused protein therapy is combined with oncolytic virus therapy to treat a subject diagnosed with melanoma/skin-cancer. In certain embodiments, the oncolytic virus comprises talimogene laherparepvec (IMLYGIC®), also referred to as T-VEC. In some embodiments, a heterodimeric Fc-fused protein comprising subunits of IL-12 is used for treating cancer (e.g., an advanced malignancy) in combination with an oncolytic virus (for example, Talimogene Laherparepvec (IMLYGIC®) or T-VEC).
(xii) Surgical Interventions

In some embodiments, the heterodimeric Fc-fused protein therapy is combined with surgical interventions, where abnormal tissue (e.g., a tumor) is surgically removed. In some embodiments, the tumor is cut from the subject's body using scalpels or other sharp tools to cut the tumor and/or surrounding tissue. In some embodiments, lasers can be used to cut abnormal tissue (e.g., a tumor). In some embodiments, surgical interventions can include the use of hyperthermia treatment, which exposes abnormal tissue (e.g., a tumor) to kill the cells of the abnormal tissue or make them more sensitive to radiation and certain chemotherapy drugs. In some embodiments, surgical interventions can include the use of photodynamic therapy, where certain drugs are activated by light to kill cancer cells. The surgical intervention can involve open surgery or minimally invasive surgery. In some embodiments, the surgical intervention can be used to remove the entire tumor, to debulk a tumor, or to ease cancer symptoms.

In some embodiments, the surgical intervention can be performed in a subject prior to administering the heterodimeric Fc-fused protein therapy. In other embodiments, the surgical intervention can be performed in a subject concurrently with the heterodimeric Fc-fused protein therapy. In other embodiments, the surgical intervention can be performed in a subject after the heterodimeric Fc-fused protein therapy.
(xiii) Cryotherapy

In some embodiments, the heterodimeric Fc-fused protein therapy is combined with cryotherapy (also called cryoablation or cryosurgery). In some embodiments, the cryotherapy is administered to a patient by applying liquid nitrogen or argon gas to destroy abnormal tissue (e.g., a tumor). In some embodiments, for tumors inside a subject's body, cryotherapy can be administered using a cryoprobe, and imaging procedures such as ultrasound or MRI can be used to guide a cryoprobe and/or to monitor freezing of target tissue.

In some embodiments, the cryotherapy can be administered to the patient prior to the heterodimeric Fc-fused protein therapy. In other embodiments, the cryotherapy can be administered to the patient concurrently with the heterodimeric Fc-fused protein therapy. In other embodiments, the cryotherapy can be administered to the subject after the heterodimeric Fc-fused protein therapy.

In certain embodiments, the method of treatment disclosed herein results in a disease response or improved survival of the subject or patient. For example, in certain embodiments, the disease response is a complete response, a partial response, or a stable disease. In certain embodiments, the improved survival is improved progression-free survival (PFS) or overall survival. Improvement (e.g., in PFS) can be determined relative to a period prior to initiation of the treatment of the present disclosure. Methods of determining disease response (e.g., complete response, partial response, or stable disease) and patient survival (e.g., PFS, overall survival) for BTC (e.g., advanced BTC, metastatic BTC), or biliary tract tumor therapy, are routine in the art and are contemplated herein. In some embodiments, disease response is evaluated according to RECIST 1.1 after subjecting the treated patient to contrast-enhanced computed tomography (CT) or magnetic resonance imaging (MRI) of the affected area (e.g., chest/abdomen and pelvis covering the area from the superior extent of the thoracic inlet to the symphysis pubis).

In some embodiments, biomarkers of immune activation are measured in order to assess biological activity. In certain embodiments, cellular parameters are assessed, e.g. peripheral blood mononuclear cell (PBMCs) for immunophenotyping (IPT) by flow cytometry. In certain embodiments, soluble factors are assessed, e.g., cytokines and chemokines in serum samples. In certain embodiments, serum levels of c-reactive protein (CRP) are assessed to determine toxicity. In certain embodiments, if the CRP concentration in the subject's blood is higher than a threshold CRP concentration, then the subject is identified as being at risk for developing an adverse drug reaction. In certain embodiments, if the CRP concentration in the subject's blood is about the same or lower than the threshold C-reactive protein concentration, the subject is not identified as being at risk for developing an adverse drug reaction. In specific embodiments, if the CRP concentration in the subject's blood is higher than the threshold CRP concentration, the administration of the pharmaceutical formulation is paused; the heterodimeric Fc-fused protein is administered at a lower dose; or a remedial action is taken to reduce or alleviate the formulation's toxicity effects in the subject.

In certain embodiments, an ex vivo IL12 response assay is used to assess activity, wherein PBMCs are collected for ex vivo stimulation followed by analysis of IFN□ production. In certain embodiments, circulating tumor (ct) deoxyribonucleic acid (DNA) may be assessed. In certain embodiments, tissue derived biomarkers are evaluated on pre-treatment and post-treatment biopsies, e.g., to investigate a possible correlation between clinical efficacy and analyzed markers. In certain embodiments, levels of PD-L1 expression are determined, e.g., using a commercially available kit (e.g., Dako PD-L₁ IHC 22C₃ pharmDx). In certain embodiments, CD3 positivity as an assay for T cell infiltration is determined by immunohistochemistry (IHC). In certain embodiments, frequency and/or localization of tumor-infiltrating leukocytes (e.g., CD8 T-cells, CD4 T-cells, T_{reg}, NK cells, macrophages [M1/2 profile]) is determined by IHC or immunofluorescence microscopy (IF). In some embodiments, a gene expression profile is performed. In some embodiments, pharmacogenomics is performed.
(m) Kits

The formulation of DF hIL12-Fc si is prepared as a lyophilized formulation or a liquid formulation. For preparing the lyophilized formulation, freeze-dried DF hIL12-Fc si is sterilized and stored in single-use glass vials. Several such glass vials are then packaged in a kit for delivering a dose to a subject diagnosed with a cancer or a tumor.

In one aspect, the present disclosure provides a kit including one or more vessels collectively including a formulation of about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, or about 10 mg of a heterodimeric Fc-fused protein. In certain embodiments, the present disclosure provides a kit including one or more vessels collectively including a formulation of about 1 mg of a heterodimeric Fc-fused protein. In certain embodiments, the present disclosure provides a kit including one or more vessels collectively including a formulation of about 1 mg of the heterodimeric Fc-fused protein comprising a first polypeptide comprising the amino acid sequence of SEQ ID NO:290 and a second polypeptide comprising the amino acid sequence of SEQ ID NO:291

In certain embodiments, the formulation is prepared and packaged as a liquid formulation and stored as about as about 0.5 mg/vial to about 1.5 mg/vial (e.g., about 0.5 mg/vial to about 1.5 mg/vial, about 0.6 mg/vial to about 1.4 mg/vial, about 0.7 mg/vial to about 1.3 mg/vial, about 0.8 mg/vial to about 1.2 mg/vial, about 0.5 mg/vial to about 1.1 mg/vial, about 0.5 mg/vial to about 1.4 mg/vial, about 0.5 mg/vial to about 1.3 mg/vial, about 0.5 mg/vial to about 1.2 mg/vial, about 0.5 mg/vial to about 1.1 mg/vial, about 0.5 mg/vial to about 1.1 mg/vial, about 0.6 mg/vial to about 1.5 mg/vial, about 0.6 mg/vial to about 1.4 mg/vial, about 0.6 mg/vial to about 1.3 mg/vial, about 0.6 mg/vial to about 1.2 mg/vial, about 0.6 mg/vial to about 1.1 mg/vial, about 0.7 mg/vial to about 1.5 mg/vial, about 0.7 mg/vial to about 1.4 mg/vial, about 0.7 mg/vial to about 1.3 mg/vial, about 0.7 mg/vial to about 1.2 mg/vial, about 0.7 mg/vial to about 1.1 mg/vial, about 0.8 mg/vial to about 1.5 mg/vial, about 0.8 mg/vial to about 1.4 mg/vial, about 0.8 mg/vial to about 1.3 mg/vial, about 0.8 mg/vial to about 1.2 mg/vial, about 0.8 mg/vial to about 1.1 mg/vial, about 0.9 mg/vial to about 1.5 mg/vial, about 0.9 mg/vial to about 1.4 mg/vial, about 0.9 mg/vial to about 1.3 mg/vial, about 0.9 mg/vial to about 1.2 mg/vial, about 0.9 mg/vial to about 1.1 mg/vial). In certain embodiments, the formulation is a liquid formulation and stored as about 1 mg/vial.

In certain embodiments, the formulation is prepared and packaged as a lyophilized formulation and stored as about 0.5 mg/vial to about 1.5 mg/vial (e.g., about 0.5 mg/vial to about 1.5 mg/vial, about 0.6 mg/vial to about 1.4 mg/vial, about 0.7 mg/vial to about 1.3 mg/vial, about 0.8 mg/vial to about 1.2 mg/vial, about 0.5 mg/vial to about 1.1 mg/vial, about 0.5 mg/vial to about 1.4 mg/vial, about 0.5 mg/vial to about 1.3 mg/vial, about 0.5 mg/vial to about 1.2 mg/vial, about 0.5 mg/vial to about 1.1 mg/vial, about 0.5 mg/vial to about 1.1 mg/vial, about 0.6 mg/vial to about 1.5 mg/vial, about 0.6 mg/vial to about 1.4 mg/vial, about 0.6 mg/vial to about 1.3 mg/vial, about 0.6 mg/vial to about 1.2 mg/vial, about 0.6 mg/vial to about 1.1 mg/vial, about 0.7 mg/vial to about 1.5 mg/vial, about 0.7 mg/vial to about 1.4 mg/vial, about 0.7 mg/vial to about 1.3 mg/vial, about 0.7 mg/vial to about 1.2 mg/vial, about 0.7 mg/vial to about 1.1 mg/vial, about 0.8 mg/vial to about 1.5 mg/vial, about 0.8 mg/vial to about 1.4 mg/vial, about 0.8 mg/vial to about 1.3 mg/vial, about 0.8 mg/vial to about 1.2 mg/vial, about 0.8 mg/vial to about 1.1 mg/vial, about 0.9 mg/vial to about 1.5 mg/vial, about 0.9 mg/vial to about 1.4 mg/vial, about 0.9 mg/vial to about 1.3 mg/vial, about 0.9 mg/vial to about 1.2 mg/vial, about 0.9 mg/vial to about 1.1 mg/vial). In certain embodiments, the formulation is a lyophilized formulation and stored as about 1 mg/vial.

In certain embodiments, the vessels collectively may include about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 12 mg, about 15 mg, about 20 mg, about 21 mg, about 24 mg, about 25 mg, about 27 mg, about 30 mg, about 35 mg, about 36 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg of the heterodimeric Fc-fused protein of the present disclosure (e.g., DF hIL12-Fc si). In certain embodiments, the vessels include about 1 mg of the heterodimeric Fc-fused protein of the present disclosure (e.g., DF hIL12-Fc si). In certain embodiments, the vessels include about 1 mg of the heterodimeric Fc-fused protein comprising a first polypeptide comprising the amino acid sequence of SEQ ID NO: 290 and a second polypeptide comprising the amino acid sequence of SEQ ID NO: 291

In certain embodiments, the formulation in the vessels may be a lyophilized formulation. In certain embodiments, the formulation in the vessels may be a liquid formulation.

In certain embodiments, the formulation may be packed in kits containing a suitable number of vials. The information on the medication may be included, which are in accordance with approved submission documents. The kit may be shipped in transport cool containers (2° C. to 8° C.) that are monitored with temperature control devices.

The formulation may be stored at 2° C. to 8° C. until use. The vials of the formulations may be sterile and nonpyrogenic, and may not contain bacteriostatic preservatives.

The description above describes multiple aspects and embodiments of the invention. The patent application specifically contemplates all combinations and permutations of the aspects and embodiments.

### EXAMPLES

The invention now being generally described, will be more readily understood by reference to the following examples, which are included merely for purposes of illustration of certain aspects and embodiments of the present invention and are not intended to limit the invention.

### Example 1—Method of Preparation

The proteins of the present invention are typically made using recombinant DNA technology. In one exemplary embodiment, a first nucleic acid sequence encoding the first polypeptide comprising a first subunit of a multisubunit protein (p40 subunit of human IL-12) fused to a first antibody Fc domain polypeptide was cloned into a first expression vector (pET-pSURE-Puro); a second nucleic acid sequence encoding a second polypeptide comprising a second, different subunit of a multisubunit protein (p35 subunit of human IL-12) fused to a second antibody Fc domain polypeptide was cloned into a second expression vector (pET-pSURE-Puro); and the first and the second expression vectors were stably transfected together into host cells (e.g., Chinese Hamster Ovary cells) to produce the heterodimeric Fc-fused proteins.

Exemplary amino acid sequence encoded by the first expression vector is shown in SEQ ID NO:292. The first expression vector encoded a first polypeptide comprising a p40 subunit of human IL-12 fused to a human IgG1 Fc sequence comprising a Y349C mutation. The first polypeptide also included K360E and K409W mutations that promote heterodimerization, and LALAPA (L234A, L235A, and P329A) mutations that reduce effector functions. In SEQ ID NO: 292, leader sequence is shown in italics, the p40 subunit sequence of human IL-12 is underlined, and the mutations are shown in bold.

(SEQ ID NO: 292)
MDMRVPAQLLGLLLLWLPGARCIWELKKDVYVVELDWYPDAPGEMVVLT
CDTPEEDGITWTLDQSSEVLGSGKTLTIQVKEFGDAGQYTCHKGGEVLSH
SLLLLHKKEDGIWSTDILKDQKEPKNKTFLRCEAKNYSGRFTCWWLTTIS
TDLTFSVKSSRGSSDPQGVTCGAATLSAERVRGDNKEYEYSVECQEDSAC
PAAEESLPIEVMVDAVHKLKYENYTSSFFIRDIIKPDPPKNLQLKPLKNS
RQVEVSWEYPDTWSTPHSYFSLTFCVQVQGKSKREKKDRVFTDKTSATVI
CRKNASISVRAQDRYYSSSWSEWASVPCSPKSSDKTHTCPPCPAPEAAGG
PSVFLFPPKPKDTLMISRTPEVTCVVVDVSHEDPEVKFNWYVDGVEVHNA
KTKPREEQYNSTYRVVSVLTVLHQDWLNGKEYKCKVSNKALAAPIEKTIS
KAKGQPREPQVCTLPPSRDELTENQVSLTCLVKGFYPSDIAVEWESNGQP
ENNYKTTPPVLDSDGSFFLYSWLTVDKSRWQQGNVFSCSVMHEALHNHY
TQKSLSLSPG

Exemplary amino acid sequence encoded from the second expression vector is shown in SEQ ID NO: 293. The second expression vector encoded a second polypeptide comprising a p35 subunit of human IL-12 fused to a human IgG1 Fc sequence comprising a S354C mutation. The second polypeptide also included Q347R, D399V, and F405T mutations that promote heterodimerization, and LALAPA (L234A, L235A, and P329A) mutations that reduce effector functions. In SEQ ID NO: 293, leader sequence is shown in italics, the p35 subunit sequence of human IL-12 is underlined, and mutations are shown in bold.

(SEQ ID NO: 293)
MDMRVPAQLLGLLLLWLPGARCRNLPVATPDPGMFPCLHHSQNLLRAVSN
MLQKARQTLEFYPCTSEEIDHEDITKDKTSTVEACLPLELTKNESCLNSR
ETSFITNGSCLASRKTSFMMALCLSSIYEDLKMYQVEFKTMNAKLLMDPK
4QIFLDQNMLAVIDELMQALNFNSETVPQKSSLEEPDFYKTKIKLCILLH
AFRIRAVTIDRVMSYLNASGGGGSGGGGSGGGGSEPKSSDKTHTCPPCPA
PEAAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALAAP
IEKTISKAKGQPREPRVYTLPPCRDELTKNQVSLTCLVKGFYPSDIAVEW
ESNGQPENNYKTTPPVLVSDGSFTLYSKLTVDKSRWQQGNVFSCSVMHEA
LHNHYTQKSLSLSPG

To achieve the highest yield of the protein, different ratios of the first and second expression vectors are explored to determine the optimal ratio for transfection into the host cells. After transfection, single clones are isolated for cell bank generation using methods known in the art, such as limited dilution, ELISA, FACS, microscopy, or Clonepix.

Clones are cultured under conditions suitable for bio-reactor scale-up and maintained expression of the proteins. The proteins are isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### Example 2—Tumor Suppression by IL-12 Fused with a Silent Fc Domain Polypeptide in a CT26 Tumor Model

This example describes relative abilities of two IL-12-Fc fusion constructs of recombinant murine IL-12 (rmIL-12) to control tumor progression in a mouse colon cancer model. The two IL-12-Fc fusion variants used in this example were mIL-12-Fc wildtype (DF-mIL-12-Fc wt), which includes wild-type murine IL-12 p40 and p35 subunits fused to the N-termini of wild-type murine IgG2a Fc domain polypeptides, and mIL-12-Fc silent (DF-mIL-12-Fc si), which includes wild-type murine IL-12 p40 and p35 subunits fused to the N-termini of murine IgG2a Fc domain polypeptides with mutations L234A, L235A, and P329G. The amino acid sequences of the proteins are shown below:

mIL-12-p40-mIgG2A-EW (first chain of DF-mIL-12-Fc
wt)
(SEQ ID NO: 286)
MWELEKDVYVVEVDWTPDAPGETVNLTCDTPEEDDITWTSDQRHGVIGS
GKTLTITVKEFLDAGQYTCHKGGETLSHSHLLLHKKENGIWSTEILKNFK
NKTFLKCEAPNYSGRFTCSWLVQRNMDLKFNIKSSSSSPDSRAVTCGMAS
LSAEKVTLDQRDYEKYSVSCQEDVTCPTAEETLPIELALEARQQNKYENY
STSFFIRDIIKPDPPKNLQMKPLKNSQVEVSWEYPDSWSTPHSYFSLKFF
VRIQRKKEKMKETEEGCNQKGAFLVEKTSTEVQCKGGNVCVQAQDRYYNS
SCSKWACVPCRVRSPRGPTIKPCPPCKCPAPNLLGGPSVFIFPPKIKDVL
MISLSPIVTCVVVDVSEDDPDVQISWFVNNVEVHTAQTQTHREDYNSTLR
VVSALPIQHQDWMSGKEFKCKVNNKDLPAPIERTISKPKGSVRAPQVYVL
PPPEEEMTEKQVTLTCMVTDFMPEDIYVEWTNNGKTELNYKNTEPVLDSD
GSYFMYSWLRVEKKNWVERNSYSCSVVHEGLHNHHTTKSFSRTPG
mIL-12-p35-mIgG2A-RVT (second chain of DF-mIL-12-
Fc wt)
(SEQ ID NO: 287)
RVIPVSGPARCLSQSRNLLKTTDDMVKTAREKLKHYSCTAEDIDHEDITR
DQTSTLKTCLPLELHKNESCLATRETSSTTRGSCLPPQKTSLMMTLCLGS
IYEDLKMYQTEFQAINAALQNHNHQQIILDKGMLVAIDELMQSLNHNGET
LRQKPPVGEADPYRVKMKLCILLHAFSTRVVTINRVMGYLSSAGGGGSG
GGGSGGGGSPRGPTIKPCPPCKCPAPNLLGGPSVFIFPPKIKDVLMISLS
PIVTCVVVDVSEDDPDVQISWFVNNVEVHTAQTQTHREDYNSTLRVVSAL
PIQHQDWMSGKEFKCKVNNKDLPAPIERTISKPKGSVRAPRVYVLPPPEE
EMTKKQVTLTCMVTDFMPEDIYVEWTNNGKTELNYKNTEPVLVSDGSYTM
YSKLRVEKKNWVERNSYSCSVVHEGLHNHHTTKSFSRTPG
mIL-12-p40-mIgG2A-EW-LALAPG (first chain of DF-
mIL-12-Fc si)
(SEQ ID NO: 288)
MWELEKDVYVVEVDWTPDAPGETVNLTCDTPEEDDITWTSDQRHGVIGS
GKTLTITVKEFLDAGQYTCHKGGETLSHSHLLLHKKENGIWSTEILKNFK
NKTFLKCEAPNYSGRFTCSWLVQRNMDLKFNIKSSSSSPDSRAVTCGMAS
LSAEKVTLDQRDYEKYSVSCQEDVTCPTAEETLPIELALEARQQNKYENY
STSFFIRDIIKPDPPKNLQMKPLKNSQVEVSWEYPDSWSTPHSYFSLKFF
VRIQRKKEKMKETEEGCNQKGAFLVEKTSTEVQCKGGNVCVQAQDRYYNS
SCSKWACVPCRVRSPRGPTIKPCPPCKCPAPNAAGGPSVFIFPPKIKDVL
MISLSPIVTCVVVDVSEDDPDVQISWFVNNVEVHTAQTQTHREDYNSTLR
VVSALPIQHQDWMSGKEFKCKVNNKDLGAPIERTISKPKGSVRAPQVYVL
PPPEEEMTEKQVTLTCMVTDFMPEDIYVEWTNNGKTELNYKNTEPVLDSD
GSYFMYSWLRVEKKNWVERNSYSCSVVHEGLHNHHTTKSFSRTPG
mIL-12-p35-mIgG2A-RVT-LALAPG (second chain of DF-
mIL-12-Fc si)
(SEQ ID NO: 289)
RVIPVSGPARCLSQSRNLLKTTDDMVKTAREKLKHYSCTAEDIDHEDITR
DQTSTLKTCLPLELHKNESCLATRETSSTTRGSCLPPQKTSLMMTLCLGS
IYEDLKMYQTEFQAINAALQNHNHQQIILDKGMLVAIDELMQSLNHNGET
LRQKPPVGEADPYRVKMKLCILLHAFSTRVVTINRVMGYLSSAGGGGSG
GGGSGGGGSPRGPTIKPCPPCKCPAPNAAGGPSVFIFPPKIKDVLMISLS
PIVTCVVVDVSEDDPDVQISWFVNNVEVHTAQTQTHREDYNSTLRVVSAL
PIQHQDWMSGKEFKCKVNNKDLGAPIERTISKPKGSVRAPRVYVLPPPEE
EMTKKQVTLTCMVTDFMPEDIYVEWTNNGKTELNYKNTEPVLVSDGSYT
MYSKLRVEKKNWVERNSYSCSVVHEGLHNHHTTKSFSRTPG

Briefly, 10⁶ CT26-Tyrp1 colon carcinoma cells were injected subcutaneously into the flank of Balb/c mice. On Day 14 after tumor inoculation, when tumor volume reached 270 mm³, the mice were randomized into different treatment groups (n=10 per group) and treated intraperitoneally with 1 μg of rmIL-12, DF-mIL-12-Fc wt at a molar dose equivalent to 1 μg rmIL-12, DF-mIL-12-Fc si at a molar dose equivalent to 1 μg rmIL-12, or 1 μg of mIgG2a isotype control once a week. Tumor growth was assessed for 60 days.

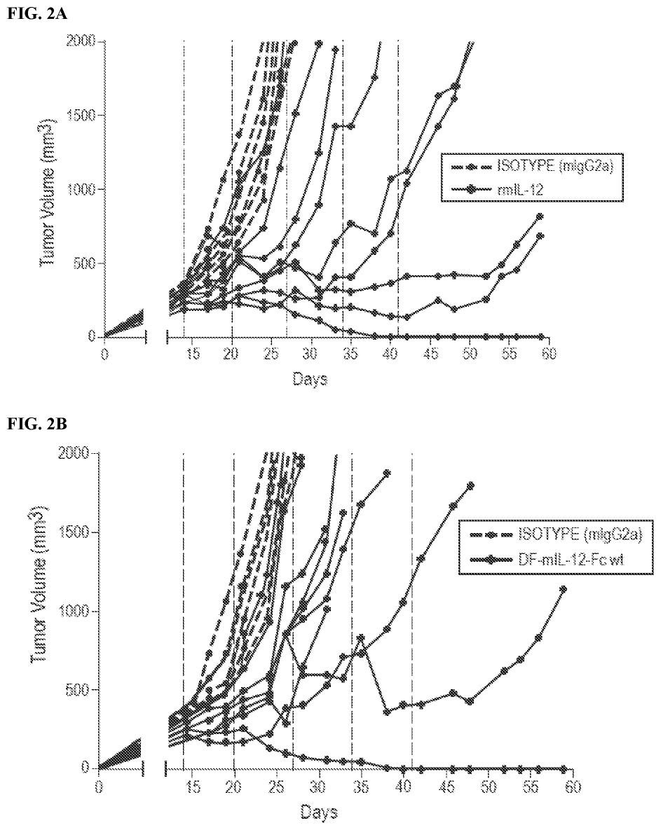

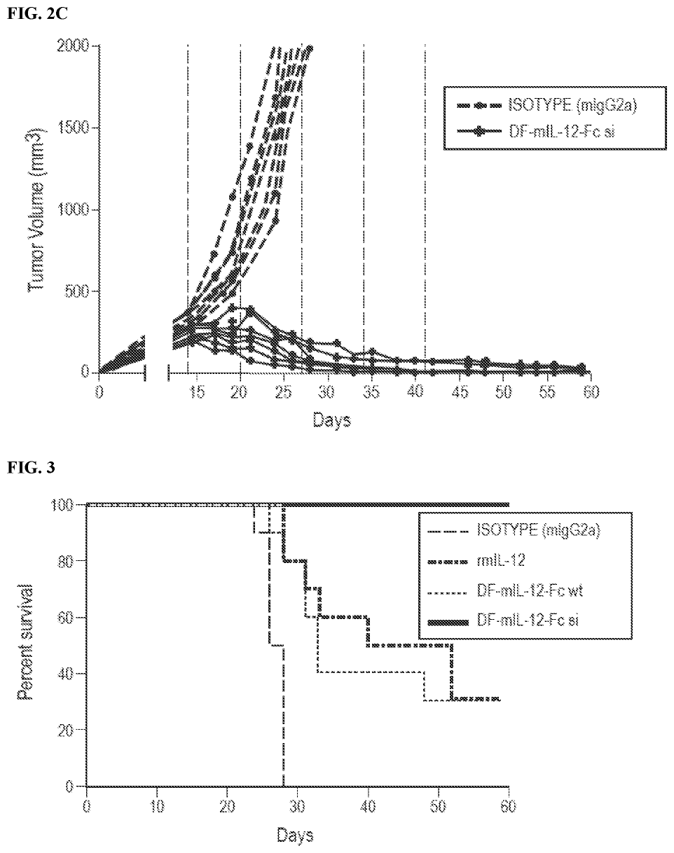

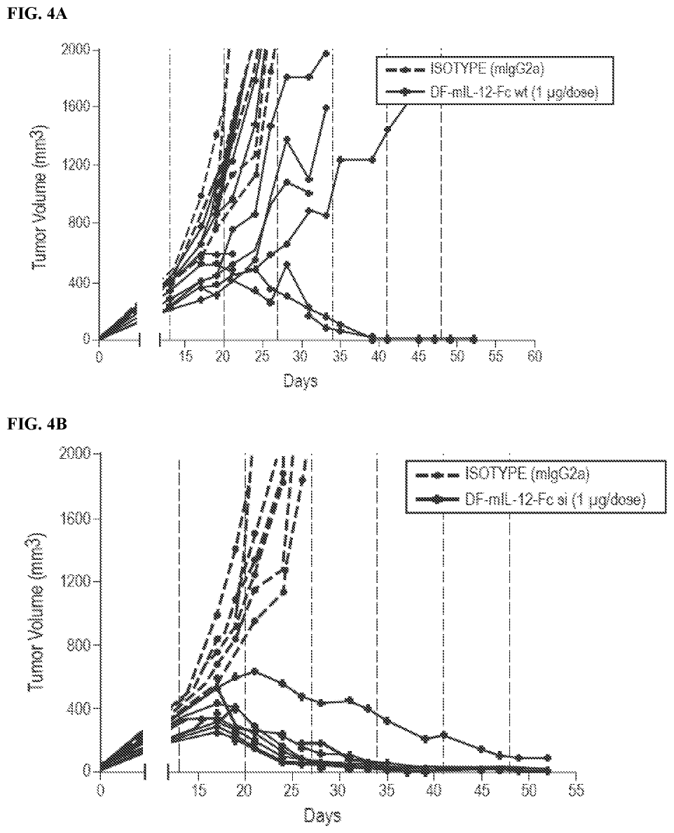

As shown in FIGS. 2A-2C, although IL-12 (FIG. 2A) and DF-mIL-12-Fc wt (FIG. 2B) were efficient in controlling tumor progression in some mice, only DF-mIL-12-Fc si induced robust tumor regression and yielded 100% complete tumor regression (FIG. 2C). Moreover, overall survival was significantly extended by the treatment of DF-mIL-12-Fc si therapy—100% of treated mice were still alive at day 60, whereas median survival times of the mice treated with isotype control, DF-mIL-12-Fc wt, and IL-12 were 27 days, 33 days, and 46 days, respectively (FIG. 3).

Next, different doses of DF-mIL-12-Fc wt and DF-mIL-12-Fc si in controlling tumor progression were compared. Briefly, 10⁶ CT26-Tyrp1 colon carcinoma cells were injected subcutaneously into the flank of Balb/c mice. On Day 14 after tumor inoculation, when tumor volume reached 300 mm³, the mice were randomized into different treatment groups (n=10 per group) and treated intraperitoneally with DF-mIL-12-Fc wt at molar doses equivalent to 1 μg or 0.1 μg rmIL-12, or DF-mIL-12-Fc si at molar doses equivalent to 1 μg or 0.1 μg IL-12 once a week. Tumor growth was assessed for 55 days.

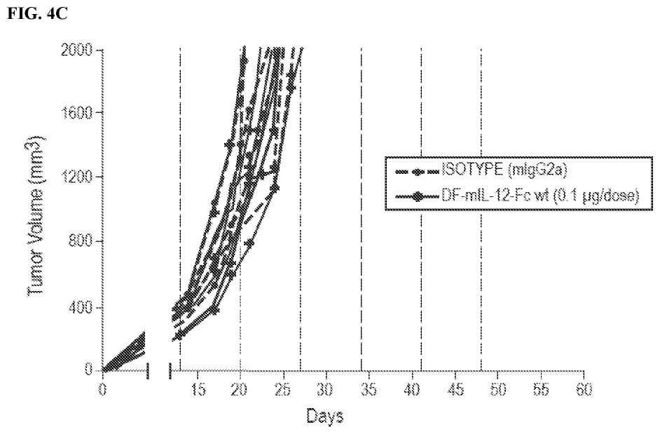

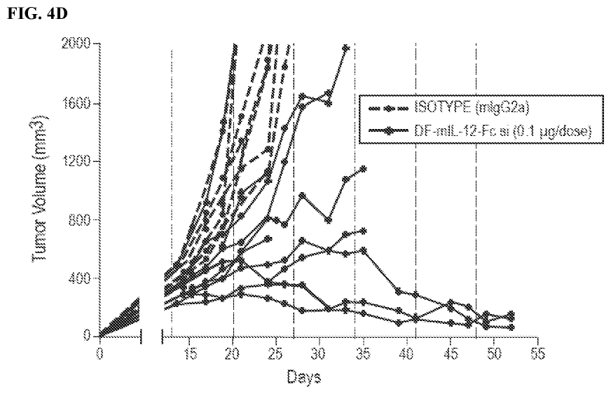

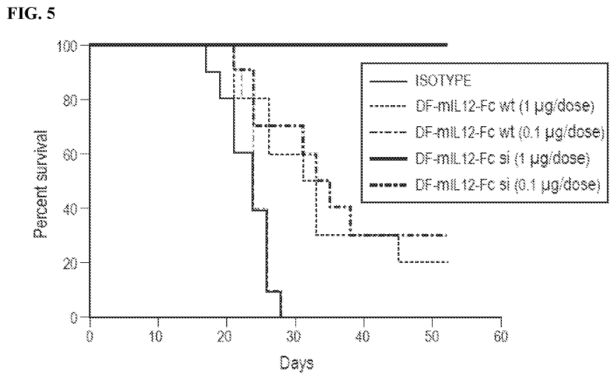

As shown in FIGS. 4A-4D, treatment with DF-mIL-12-Fc wt led to reduced tumor progression in some mice and complete regression in two mice at the 1 μg rmIL-12 molar equivalents dose (FIG. 4A), but no tumor suppression was observed at the 0.1 μg IL-12 molar equivalents dose (FIG. 4C). By contrast, the DF-mIL-12-Fc si treatment at the 1 μg IL-12 molar equivalents dose yielded 100% complete tumor regression (FIG. 4B) and induced a robust delay in tumor growth at the lower dose of 0.1 μg IL-12 molar equivalents (FIG. 4D). The median survival of the mice treated with 1 μg IL-12 molar equivalents of DF-mIL-12-Fc wt was 32 days, similar to the 34 days of median survival of the mice treated with 0.1 μg IL-12 molar equivalents of DF-mIL-12-Fc si, suggesting that DF-mIL-12-Fc si was 10-fold more potent than its wildtype variant (FIG. 5). DF-mIL-12-Fc wt was not efficient at the dose of 0.1 μg IL-12 molar equivalents, and showed the same median survival of 24 days as the isotype treated group.

Next, in vivo efficacy for different routes of administering DF-mIL-12-Fc si were compared. Briefly, 10⁶ CT26-Tyrp1 colon carcinoma cells were injected subcutaneously into the flank of Balb/c mice. On Day 14 after tumor inoculation, when tumor volume reached 270 mm³, the mice were randomized into different treatment groups (n=10 per group) and treated either intraperitoneally or subcutaneously with DF-mIL-12-Fc si at a molar dose equivalent to 1 μg IL-12 or molar equivalent of mIgG2a isotype control once a week. Tumor growth was assessed for over 60 days.

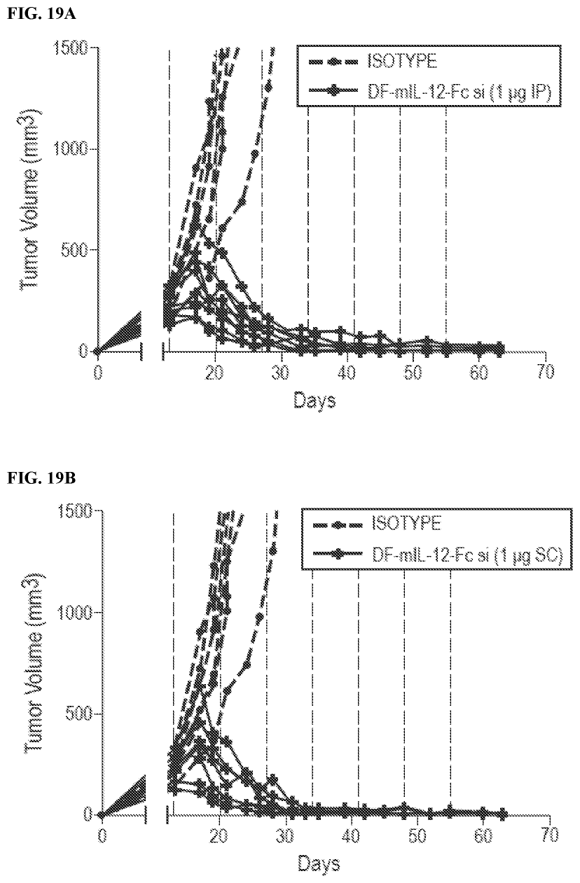

As shown in FIGS. 19A-19B, both intraperitoneal (FIG. 19A) and subcutaneous (FIG. 19B) administration of DF-mIL-12-Fc si induced robust tumor regression and yielded 100% complete tumor regression. Thus, DF-mIL-12-Fc si treatment demonstrated efficacy using various routes of administration.

### Example 3—Tumor Suppression by IL-12 Fused with a Silent Fc Domain Polypeptide in a B16F10 Tumor Model

This example describes relative abilities of DF-mIL-12-Fc wt and DF-mIL-12-Fc si in controlling tumor progression in a mouse melanoma model. Briefly, 10⁶ B16F10 melanoma cells were injected subcutaneously into C57BL/6 mice. On Day 8 after tumor inoculation, when tumor volume reached 250 mm³, the mice were randomized into different treatment groups (n=10) and treated with 0.5 μg of IL-12, DF-mIL-12-Fc wt at a molar dose equivalent to 0.5 μg IL-12, DF-mIL-12-Fc si at a molar dose equivalent to 0.5 μg IL-12, or 0.5 μg of mIgG2a isotype control once a week. Tumor growth was assessed for 32 days.

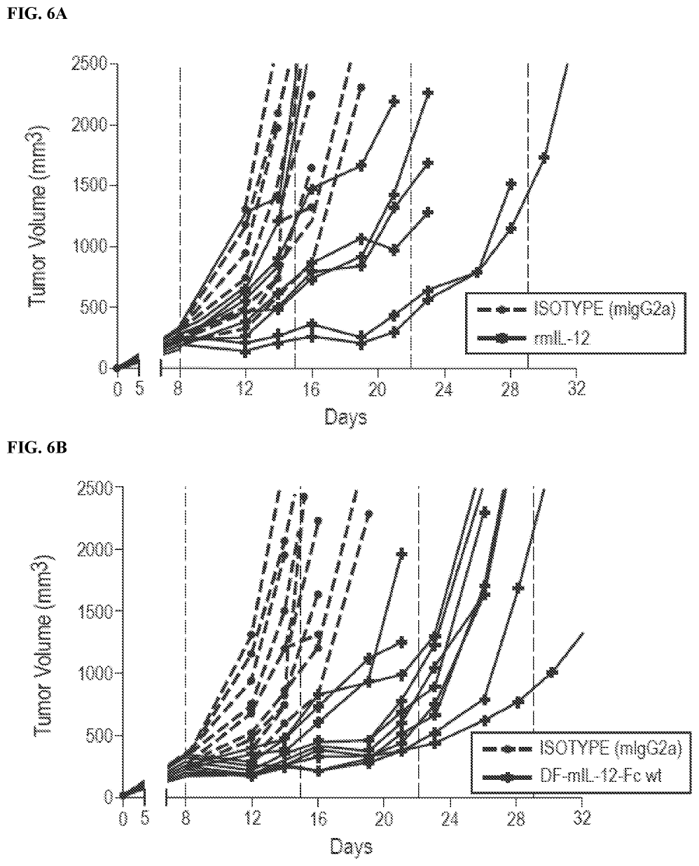

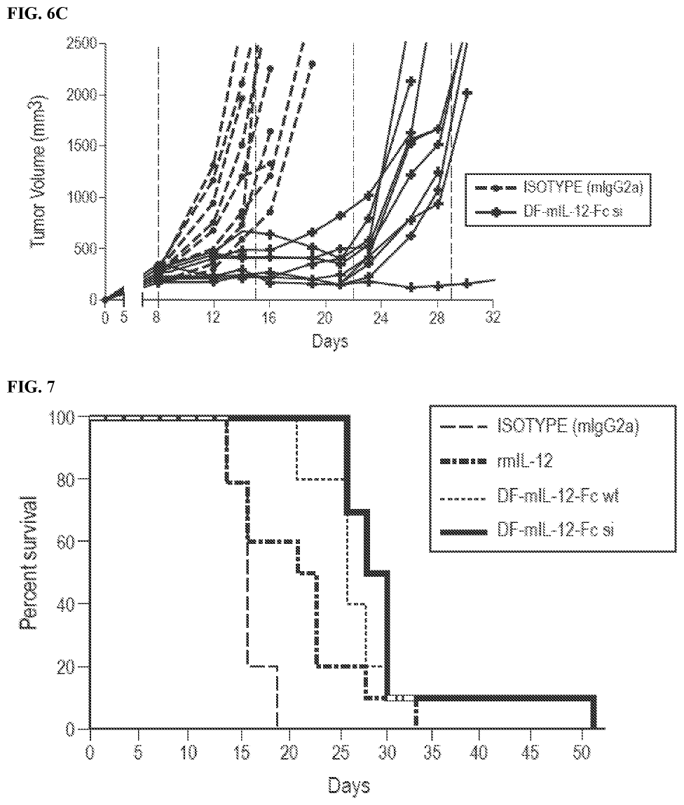

As shown in FIGS. 6A-6C, although each of the IL-12-Fc constructs tested delayed tumor progression, DF-mIL-12-Fc si was the most efficient in controlling tumor growth. Median survival time of the mice treated with DF-mIL-12-Fc si was 29 days, which was longer than the median survival times of the mice treated with isotype control, DF-mIL-12-Fc wt, and IL-12, which were 16 days, 26 days, and 22 days, respectively (FIG. 7).

Next, different doses of DF-mIL-12-Fc wt and DF-mIL-12-Fc si in controlling tumor progression were compared. Briefly, 10⁶ B16F10 melanoma cells were injected subcutaneously into the flank of C57BL/6 mice. On Day 8 after tumor inoculation, the mice were randomized into different treatment groups (n=10 per group) and treated intraperitoneally with 0.5 μg or 0.1 μg IL-12 molar equivalents of DF-mIL-12-Fc wt, or 0.5 μg or 0.1 μg IL-12 molar equivalents of DF-mIL-12-Fc si once a week. Tumor growth was assessed for 30 days.

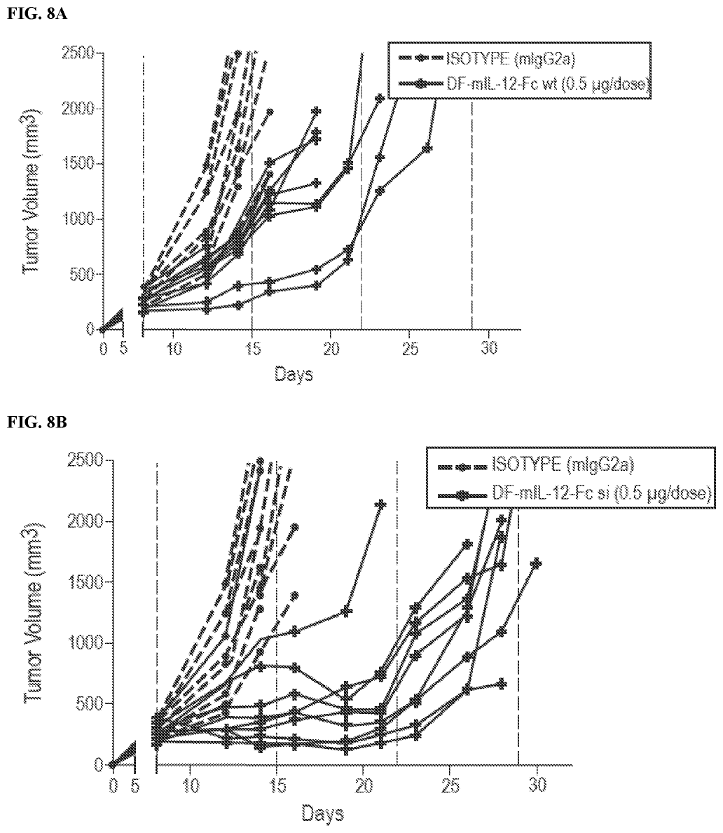

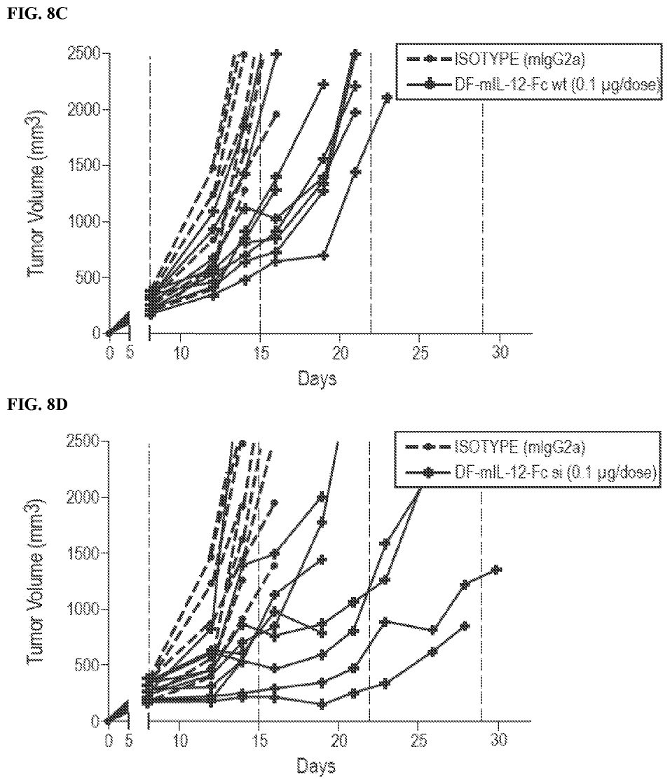

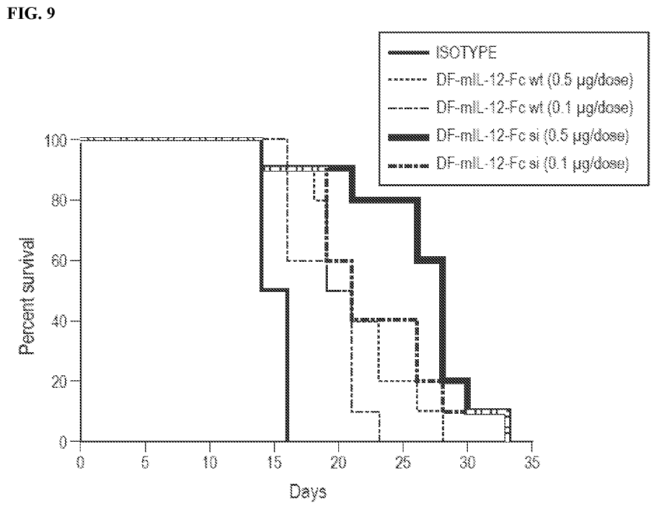

As shown in FIGS. 8A-8D, DF-mIL-12-Fc si was superior to DF-mIL-12-Fc wt in suppression of tumor growth at both doses. Moreover, at each dose, the median survival of the mice treated with DF-mIL-12-Fc wt was 20 days. By contrast, the median survival of the mice treated with 0.1 μg IL-12 molar equivalents of DF-mIL-12-Fc si was 21 days, and the median survival of the mice treated with 0.5 μg IL-12 molar equivalents of DF-mIL-12-Fc si was 28 days (FIG. 9). These results demonstrated that a high dose (0.5 μg IL-12 molar equivalents) of DF-mIL-12-Fc si significantly increased the survival of mice compared to its wildtype counterpart or isotype control.

Next, single dose administrations of the DF-mIL-12-Fc si treatment was compared to the weekly treatments previously described. Briefly, 10⁶ B16F10 melanoma cells were injected subcutaneously into C57BL/6 mice. On Day 8 after tumor inoculation, when tumor volume reached 200 mm³, the mice were randomized into different treatment groups (n=10) and treated with DF-mIL-12-Fc si at a molar dose equivalent to 0.5 μg IL-12 or molar equivalent of mIgG2a isotype control once a week. Tumor growth was assessed for 39 days.

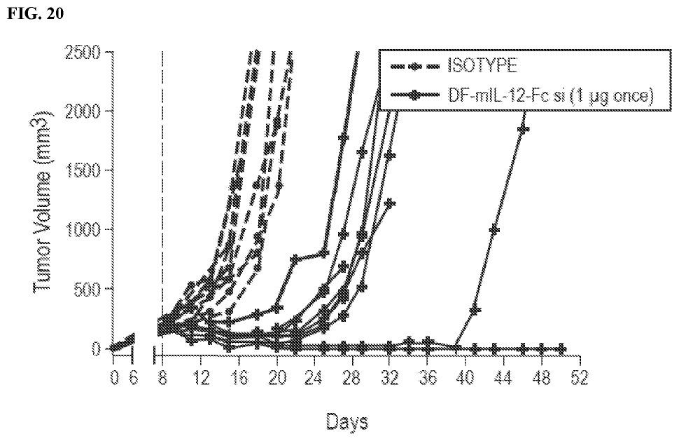

As shown in FIG. 20, a single administration of DF-mIL-12-Fc si resulted in reduced tumor outgrowth in 100% of mice, although tumor outgrowth occurred sooner when compared to weekly administrations (FIG. 6C). Additionally, mice demonstrated transient weight loss, but after the first dose only (data not shown). Accordingly, a single administration of DF-mIL-12-Fc si demonstrated initial efficacy in a hard-to-treat tumor model, although subsequent weekly administrations are better at delaying tumor outgrowth in this model.

Next, in vivo efficacy for different routes of administering DF-mIL-12-Fc si were compared. Briefly, 10⁶ B16F10 melanoma cells were injected subcutaneously into C57BL/6 mice. On Day 7 after tumor inoculation, when tumor volume reached 260 mm³, the mice were randomized into different treatment groups (n=10) and treated either intraperitoneally or subcutaneously with DF-mIL-12-Fc si at a molar dose equivalent to 1 μg IL-12 or molar equivalent of mIgG2a isotype control once a week. Tumor growth was assessed for 40 days.

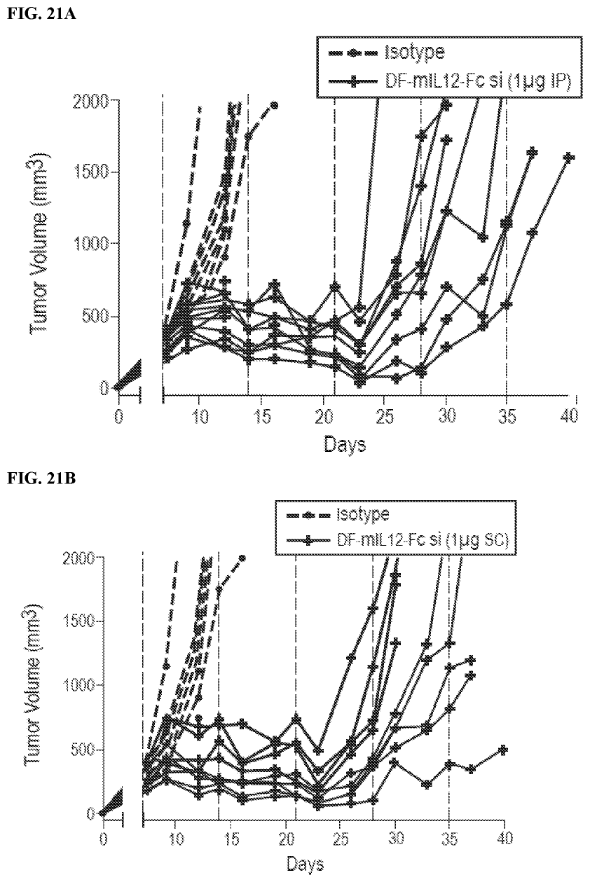

As shown in FIGS. 21A-21B, both intraperitoneal (FIG. 21A) and subcutaneous (FIG. 21B) administration of DF-mIL-12-Fc si induced tumor regression in 100% of mice. Thus, DF-mIL-12-Fc si treatment demonstrated efficacy using various routes of administration.

### Example 4—In Vitro Potency of DF-hIL-12-Fc Wt and rhIL-12

The potency of DF-hIL-12-Fc si in comparison to rhIL-12 was assessed using in vitro bioassays.

IL-12 potency was assessed using a HEK-Blue IL-12 reporter assay. IL-12R+ HEK-Blue reporter cells (InvivoGen) were harvested from culture and adjusted to 1×10⁶ cells/mL in culture media. DF-hIL-12-Fc si (DF IL-12-Fc) and recombinant human IL-12 (rhIL-12; PeproTech) were diluted in media. 100 μL of PBMC suspension was mixed with 100 μL of diluted test article and incubated for 48 hours. The supernatant was harvested and engagement of IL-12 receptor and signaling components stably expressed by the reporter cells was detected by measurement of secreted embryonic alkaline phosphatase from the cells following manufacturer instructions. Briefly, 25 μL of sample supernatant was mixed with 200 μL of QUANTI-Blue reagent and incubated in the dark at RT for 10 minutes. The plate was then read with a SpectraMax i3x plate-reader at 620 nM and optical density reported to represent relative IL-12 activity.

As shown in FIG. 10A, production of SEAP by IL-12R+HEK reporter cells increased with increasing concentrations of DF-hIL-12-Fc si or rhIL-12. The measured IL-12 responses in the HEK-Blue reporter assay were comparable between DF-hIL-12-Fc si and rhIL-12 at the concentrations examined.

Next, IL-12 potency was assessed by quantifying IFNγ production from human PBMCs. PBMCs were isolated from human peripheral blood buffy coats using density gradient centrifugation and adjusted to 1×10⁶ cells/mL in culture media. DF-hIL-12-Fc si and recombinant human IL-12 (rhIL-12) were diluted in media. 100 μL of PBMC suspension was mixed with 100 μL of diluted test article and incubated for 48 hrs. The supernatant was harvested and IFNγ was quantified using a Human IFN-7 ELISA MAX kit (BioLegend). After development of the IFNγ ELISA plates, they were read using a SpectraMax i3x instrument at 450 nm with a background subtraction at 540 nm. IFNγ content in sample wells was approximated by interpolating sample readings from the assay standard curve.

As shown in FIG. 10B, IFNγ production increased when human PBMCs were cultured with DF-hIL-12-Fc si or rhIL-12, with concurrent treatment with 5 μg/ml of PHA to amplify the magnitude of IFNγ responses. IFN-7 production following IL-12 stimulation was comparable between DF-hIL-12-Fc si and rhIL-12 at the concentrations examined.

Accordingly, although the EC50 values with the two cell types and stimulation conditions differed by over an order of magnitude, comparable activity of DF-hIL-12-Fc si and rhIL-12 was demonstrated in both assays suggesting the potency of the DF-hIL-12-Fc si construct exhibits similar potency to that of native recombinant human IL-12.

### Example 5—IL-12, DF-hIL-12-Fc Si and IFNγ Concentrations in Monkey Plasma Following IV Infusion of DF-hIL-12-Fc Si or rhIL-12

The pharmacodynamics (PD) and pharmacokinetics (PK) were assessed in cynomolgus monkeys following IV infusion of DF-hIL-12-Fc si or rhIL-12.

Cynomolgus monkeys were administered DF-hIL-12-Fc si and recombinant human IL-12 at 10 μg/kg by IV-infusion.

An immunoassay was used to detect DF-hIL-12-Fc si and Human IL-12 based on a Quantikine ELISA Human IL-12 p70 Immunoassay kit: This assay employed the quantitative sandwich enzyme immunoassay technique. A monoclonal antibody specific for human IL-12 p70 was used as a solid phase capture and detection was accomplished using an antibody HRP-tagged reporter. Standards and QCs spiked with rhIL-12 or DF-hIL-12-Fc si reference standard, along with test samples were pipetted into the wells of microtiter plate and any IL-12 p70 present in the samples were bound by the immobilized antibody, on the solid phase. Unbound substances were washed away and the enzyme-linked polyclonal antibody specific for human IL-12 p70 was added to the wells. Unbound antibody-enzyme reagents were washed away and TMB substrate was added to each well. The resulting enzyme reaction yields a blue product that turns yellow when an acid stop solution is added. The intensity of the color measured in each well is directly proportional to the amount of rhIL-12 or DF-hIL-12-Fc si is bound in the initial step. Plates were read at 450 nm with a reference of 540 nm on a SpectraMax microplate reader with data collection software, SoftMax Pro Enterprise version 4.6. Data was converted into a text file and imported/processed in Watson LIMS v.7.2.0.02. Regression was performed using a Logistic (Auto Estimate) curve fitting with a weighting factor of 1.

An immunoassay (meso scale discovery (MSD)—an ELISA like immunoassay) was also used to detect DF-hIL-12-Fc si that involved coating an untreated MSD microtiter plate with monkey-adsorbed goat anti-human IgG and incubating at room temperature. The plate was washed, blocked, washed, and incubated with standard curve and quality control samples spiked with DF-hIL-12-Fc si reference standard, along with test samples. After this incubation, the plate was washed and biotin anti-human IL-12/IL-23 p40 was added to the plate as the primary detection antibody. After another wash step, streptavidin-conjugated Sulfo-Tag was added as the secondary detection antibody. The plate was washed a final time, MSD Read Buffer T was added to the plate, and the plate was read using a MSD Sector Imager S600. Raw MSD data was exported into a text file, which was then converted into a Watson LIMS compatible file using a programmed Excel spreadsheet, which was custom designed at Envigo. Data was imported and regressed in Watson LIMS Software v.7.2.0.02.

A meso scale discovery method was performed for the relative quantitative measurement of NHP proinflammatory biomarkers in cynomolgus monkey plasma. The methed used a sandwich immunoassay procedure for the relative quantitative measurement of Pro-inflammatory Panel 1 Biomarkers: IFNγ, IL-10, IL-2, IL-6 IL-8, and IL-10 in cynomolgus monkey K2 EDTA plasma (referred to as monkey plasma). The method is based on MSD non-human primate (NHP) kits for V-PLEX and V-PLEX Plus, Catalog No. K15056D-1, K15056D-2, K15056D-4, K15056D-6, K15056G-1, K15056G-2, K15056G-4, K15056G-6. The method employs human capture and detection antibodies that react with cynomolgus monkeys. The kit provides plates pre-coated with capture antibodies on independent well-defined spots in each well of a 96-well multi-spot plate. The plate was incubated with monkey plasma samples, washed and then incubated with detection antibodies (specific for each analyte) that are conjugated with electrochemiluminescent (ECL) labels (MSD SULFO-TAG). Analytes in the sample bind to capture antibodies immobilized on the working electrode surface; recruitment of the detection antibodies by the bound analytes completes the sandwich. The plate was washed and an MSD Read Buffer was added to create the appropriate chemical environment for electrochemiluminiscence (ECL). The plate was loaded into an MSD Sector Imager 600 (SI600) instrument where a voltage was applied to the plate electrodes causing the captured labels to emit light. The instrument measures the intensity of emitted light in terms of Relative Light Units (RLU) to provide a relative quantitative measure of analytes in the sample. Raw RLU data was exported into a text file, which then was converted into a Watson LIMS compatible file using a programmed Excel spread sheet, which was custom designed at Envigo. Data was subsequently imported and regressed in Watson LIMS Software v.7.2.0.02.

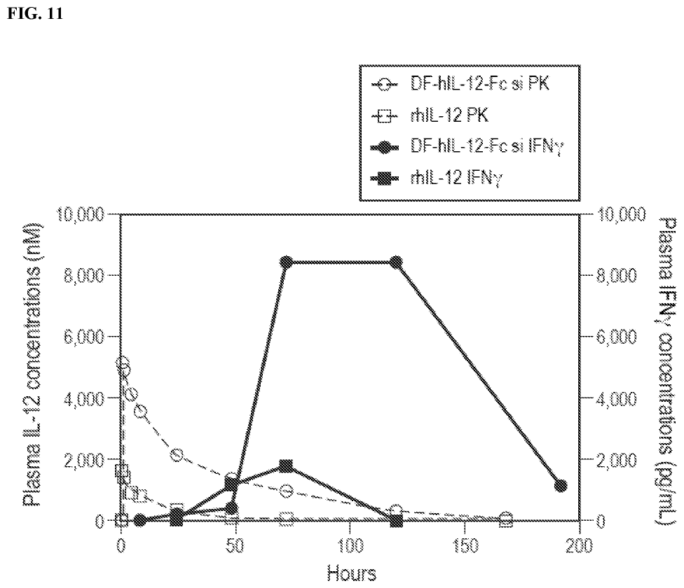

FIG. 11 shows the relative plasma concentrations of DF-hIL-12-Fc si and recombinant human IL-12 over time following IV-administration. The data indicate that concentrations of DF-hIL-12-Fc si and rhIL-12 decreased over time, as expected. However, DF-hIL-12-Fc si demonstrated a prolonged half-life and overall greater exposure compared to rhIL-12 over the time course.

FIG. 11 also shows the relative concentrations of IFNγ (PD) in monkey plasma following IV-administration. The data indicate that the pharmacodynamics of DF-hIL-12-Fc si and rhIL-12, as assessed by IFNγ production, both demonstrated activity following IV-administration. However, DF-hIL-12-Fc si demonstrated a higher peak activity and a longer duration compared to rhIL-12.

### Example 6—Pharmacological Characterization of the Mouse Surrogate DF-mIL-12-Fc Si

The serum half-life and in vivo pharmacodynamics of a half-life prolonged murine IL-12 variant, designated DF-mIL-12-Fc si, was examined.

An equivalent molar amount of DF-mIL-12-Fc si, corresponding to 1 μg IL-12, was intravenously injected in non-tumor bearing Balb/c mice and PK/PD characteristics were compared to IL-12. Naïve Balb/c (n=6) were injected intravenously with 1 μg DF-mIL-12-Fc si and IL-12 (equivalent molar to 1 μg IL-12). Blood was sampled at 0.017, 0.5, 3, 6, 24, 48, 72, 96, 144 and 219 hours post-injection. IL-12 and IFNγ levels in serum were analyzed by ELISA, as previously described.

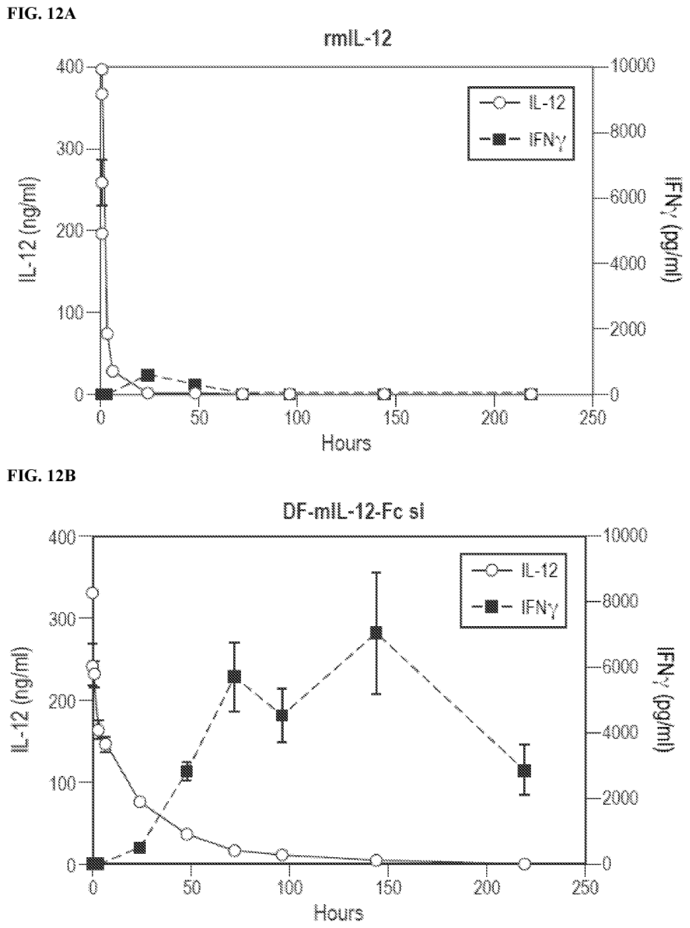

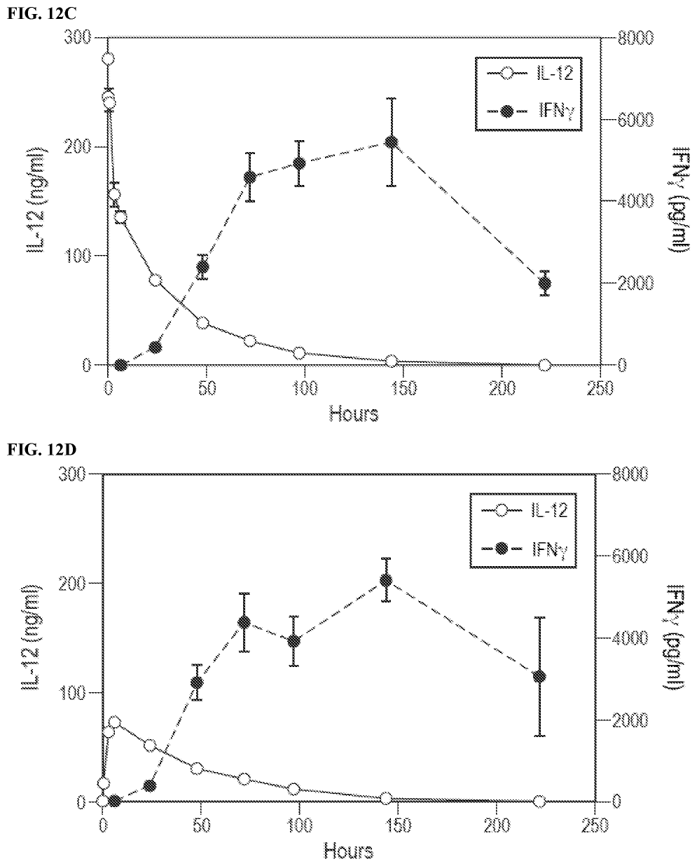

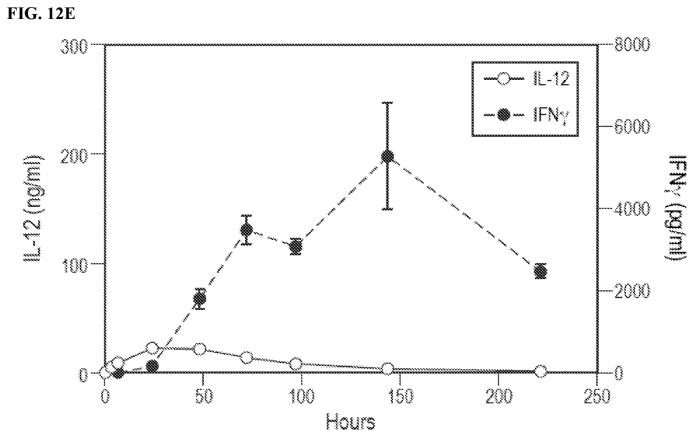

As shown in FIGS. 12A and 12B and quantified in Table 15, DF-mIL-12-Fc si showed protracted serum half-life of approximately 30 hours (FIG. 12B, DF-mIL-12-Fc si T_{1/2}=29.85 hours), which was 5 times longer than that of IL-12 (FIG. 12A; IL-12 T_{1/2}=6.05 hours). In addition to an extended half-life, DF-mIL-12-Fc si-mediated IFNγ production (AUC=916654) was also prolonged compared to IL-12 (AUC=20304).

Next, the PK/PD properties for different routes of administering DF-mIL-12-Fc si were compared. An equivalent molar amount of DF-mIL-12-Fc si, corresponding to 1 μg IL-12, was injected as a single dose in non-tumor bearing Balb/c mice by intravenous, intraperitoneal, or subcutaneous administration and PK/PD characteristics were assessed, as described.

As shown in FIGS. 12C-12E and quantified in Table 16, intravenous (FIG. 12C), intraperitoneal (FIG. 12D), or subcutaneous (FIG. 12E) administration all resulted in DF-mIL-12-Fc si-mediated IFNγ production comparable across the different routes of administration. Notably, subcutaneous administration resulted in a lower IL-12 Cₘₐₓ. Accordingly, the pharmacokinetic properties (e.g., IL-12 concentration) of DF-mIL-12-Fc si administration varied depending on the route of administration, while the pharmacodynamic properties (IFNγ production) remained protracted and relatively comparable across the different routes.

TABLE 15
Pharmacological characteristics of DF-mIL-12-Fc si and rmIL-12

| | IL-12 | | | | | IFNγ |
|---|---|---|---|---|---|---|
| | T_{1/2} | Span | Tₘₐₓ | Cₘₐₓ | AUC | AUC |
| rmIL-12 | 6.05 | 9.15 | 0.13 | 358.65 | 999.81 | 20304 |
| DF-mIL-12-Fc | 29.85 | 6.92 | 0.19 | 258.20 | 5636.49 | 916654 |

TABLE 16
Pharmacological characteristics of
DF-mIL-12-Fc si via IV, IP, and SC

| | T_{1/2} | Span | Tₘₐₓ | Cₘₐₓ | AUC |
|---|---|---|---|---|---|
| Intravenous | 31.5 | 6.4 | 0.2 | 257.2 | 5985.6 |
| Intraperitoneal | 34.70 | 5.19 | N/A | 75.09 | 3964.21 |
| Subcutaneous | 37.5 | 4.1 | 36.0 | 23.1 | 1938.1 |

### Example 7—Combination of DF-mIL-12-Fc Si and PD-1 Blockade in B16F10 Mouse Model

Combination therapy of DF-mIL-12-Fc si and PD-1 blockade was performed to analyze whether anti-tumor immune response can be amplified in established B16F10 tumors.

C57BL/6 mice were injected with 10⁶ B16F10 melanoma cells subcutaneously into the flank of mice. On Day 8 after tumor inoculation, mice were randomized (n=10 per group). When average tumor volume reached ˜245 mm³, mice were treated intraperitoneally with 0.5 μg isotype control, 0.5 μg DF-mIL-12-Fc si, 200 μg anti-PD-1 clone RMP1-14, or combined DF-mIL-12-Fc si/anti-PD-1. Animals were injected once a week with DF-mIL-12-Fc si and twice weekly with anti-PD-1. Tumor growth was assessed for 60 days, and survival and body weight was monitored.

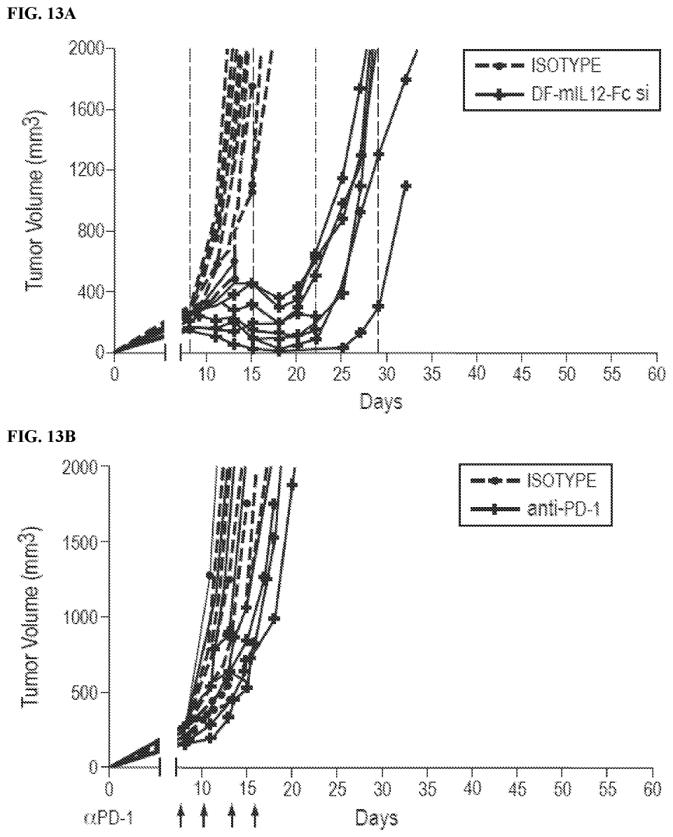

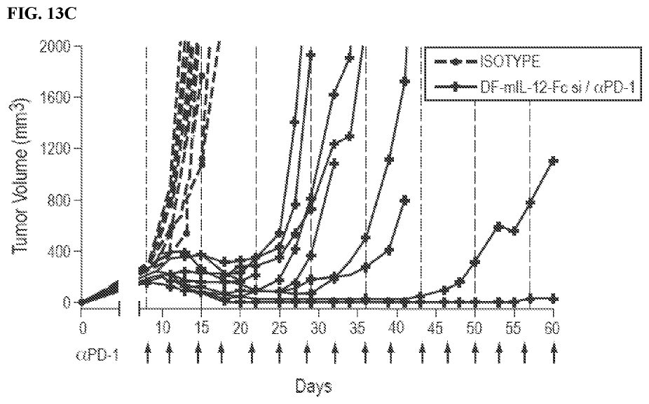

As shown in FIGS. 13A-13C, while administration of DF-mIL-12-Fc si alone delayed tumor regression (FIG. 13A) and PD-1 alone had a minimal effect on tumor growth (FIG. 13B), the combination of DF-mIL-12-Fc si with PD-1 blockade further delayed tumor growth (FIG. 13C), suggesting anti-PD-1 treatment further amplified anti-tumor responses to DF-mIL-12-Fc si treatment.

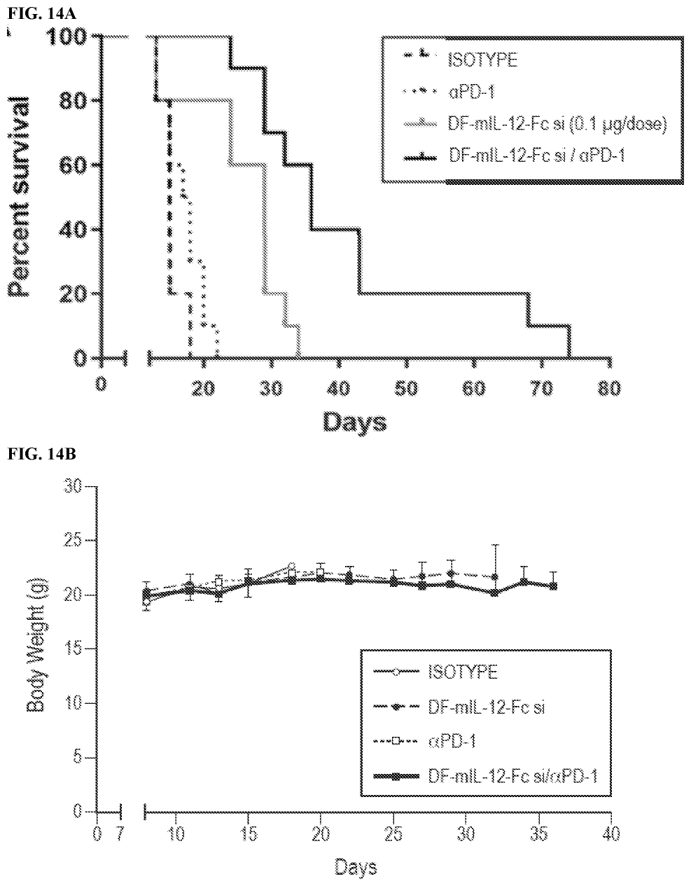

As shown in FIGS. 14A and 14B, overall survival with DF-mIL-12-Fc si therapy and in combination with PD-1 blockade was extended showing a median survival of 29 days (DF-mIL-12-Fc si monotherapy) and 36 days (combination) compared to 15 days of isotype and 17.5 days of 200 μg anti-PD-1 treated mice (FIG. 14A). Notably, despite the high response rate, the regimen of DF-mIL-12-Fc si and combination therapy appeared to be well tolerated by B16F10 tumor-bearing mice (FIG. 14B).

Accordingly, a combination therapy of DF-mIL-12-Fc si and PD-1 blockade demonstrated improved efficacy compared to either treatment alone.

### Example 8—Combination of DF-mIL-12-Fc Si and with mcFAE-C26.99 TriNKETs in B16F10 Mouse Model

Combination therapy of DF-mIL-12-Fc si and mcFAE-C26.99 TriNKETs was performed to analyze whether anti-tumor immune response can be amplified in established B16F10 tumors.

C57BL/6 mice were injected with 10⁶ B16F10 melanoma cells subcutaneously into the flank of the mice. On Day 7 after tumor inoculation mice were randomized (n=10 per group). When tumor average reached 200 mm³, mice were treated intraperitoneally with 150 μg isotype control, or 0.5 μg DF-mIL-12-Fc si, 150 μg TriNKET, or the combination DF-mIL-12-Fc si/TriNKET. Tumor growth was assessed for 60 days, and survival and body weight was monitored.

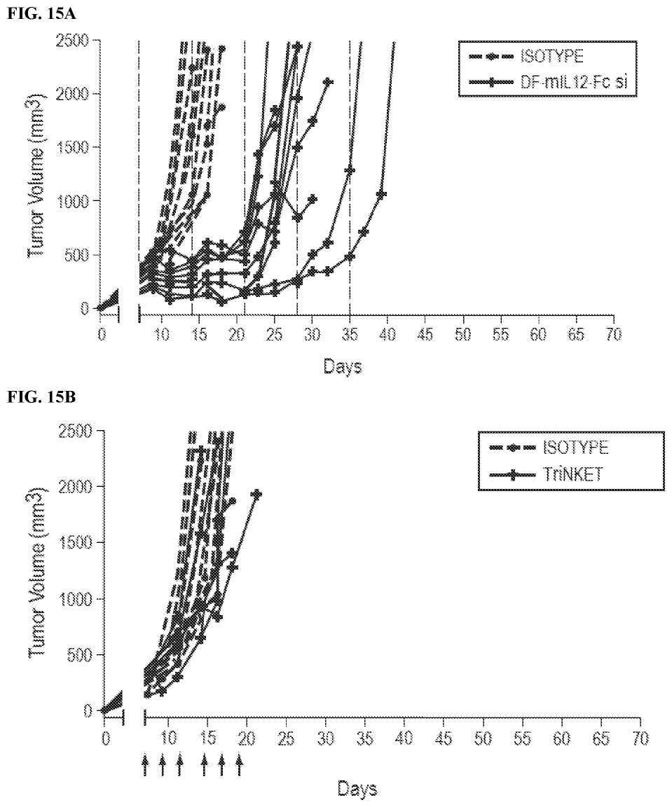

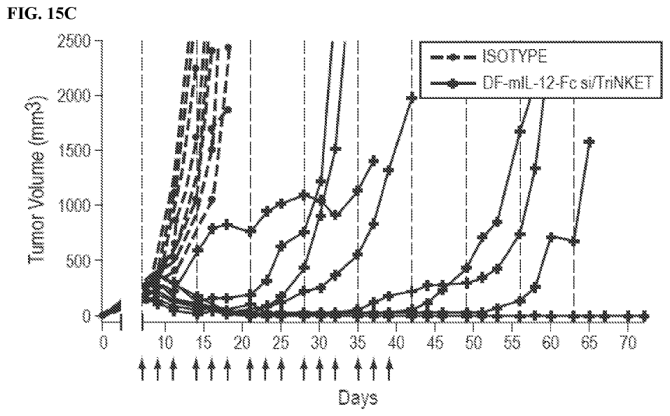

As shown in FIG. 15A, monotherapy with DF-mIL-12-Fc si led to reduced tumor growth. Treatment with mcFAE-C26.99 TriNKET as single agent at a starting tumor volume of 200 mm³ did not result in delayed tumor progression (FIG. 15B). In contrast, the combination of DF-mIL-12-Fc si with mcFAE-C26.99 further enhanced anti-tumor responses in comparison to DF-mIL-12-Fc si alone (FIG. 15C) and resulted in 30% complete responders (CR) (n=3), suggesting TriNKET treatment further amplified anti-tumor responses to DF-mIL-12-Fc si treatment.

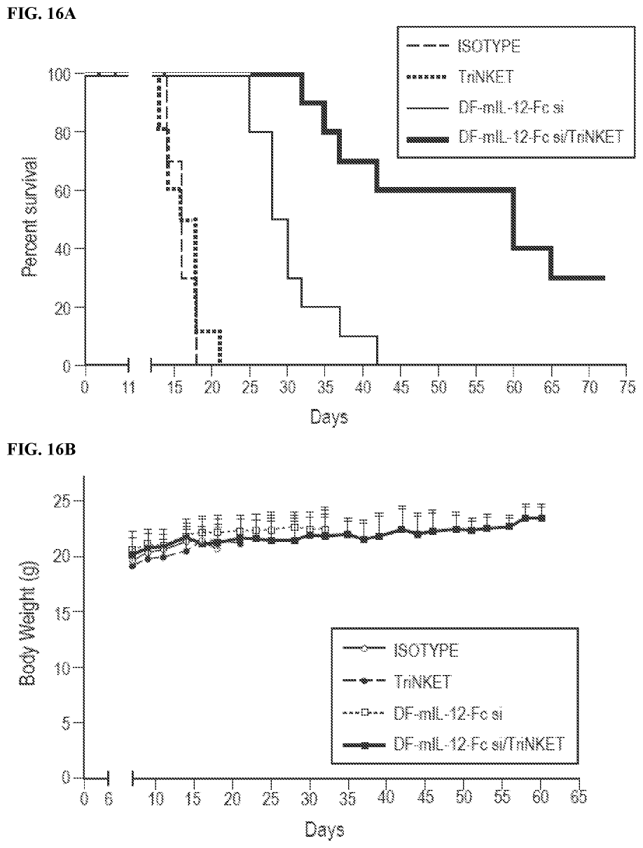

As shown in FIG. 16A, overall survival with DF-mIL-12-Fc si therapy and in combination with mcFAE-C26.99 TriNKET was extended showing a median survival of 29 days (DF-mIL-12-Fc si monotherapy) and 60 days (TriNKET combination) compared to 16 days of isotype and 17 days of TriNKET treated mice. Notably, despite the high response rate, the regimen of DF-mIL-12-Fc si and combination therapy appeared to be well tolerated by B16F10 tumor-bearing mice (FIG. 16B).

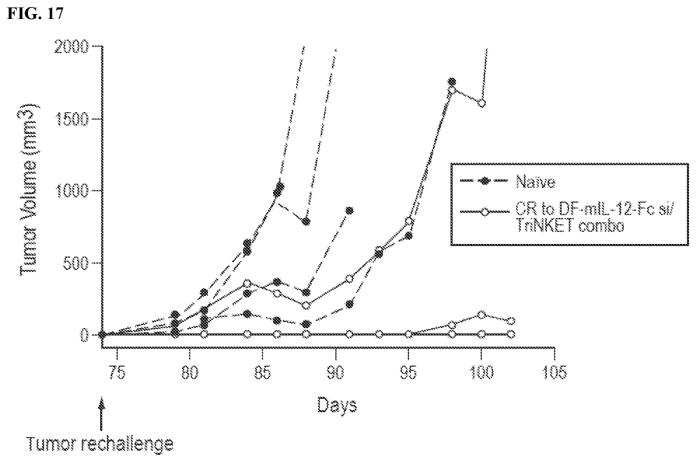

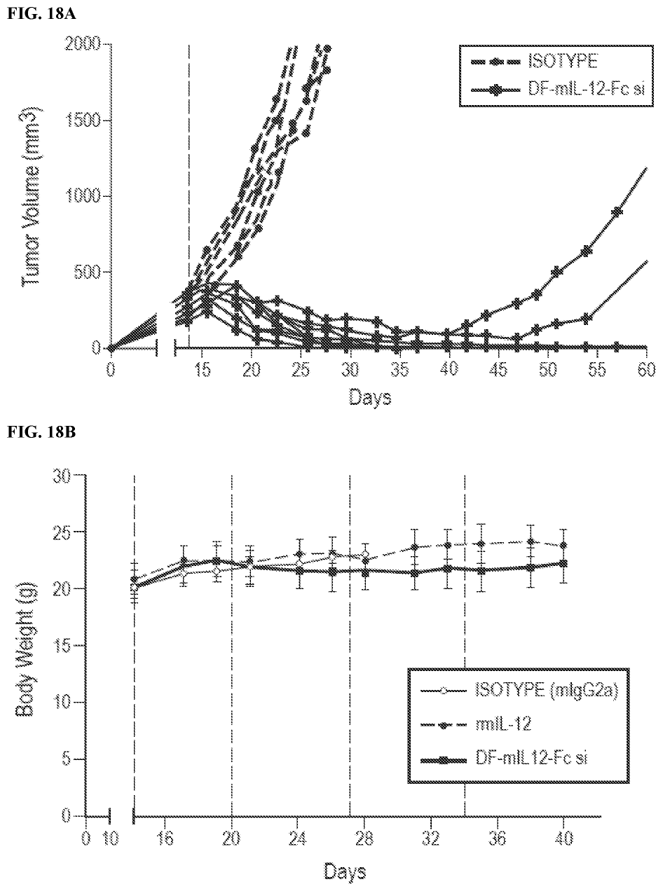

The three complete responders (the CRs from the experiment described above and the data presented in FIG. 15C) were re-challenged with 2×10⁶ B16F10 melanoma cells 72 days after first tumor inoculation. Age-matched naïve C57BL/6 mice were used as control group. 1 out of 3 mice from the initial DF-mIL-12-Fc si/TriNKET combo treated group remained tumor-free, another mouse showed tumor formation starting at day 95 and the tumor progression of the third mouse was similar to the age-matched control group (FIG. 17), suggesting the formation of immunological memory with combination therapy.

Accordingly, a combination therapy of DF-mIL-12-Fc si and TriNKETs demonstrated improved efficacy compared to either treatment alone, including demonstrating a complete, durable response in a population of mice.

### Example 9—Treatment with DF-mIL-12-Fc Si Promotes Complete Recovery in CT26 Tumor Model

This example shows that treatment with DF-mIL-12-Fc si promotes recovery in mice bearing CT26 tumors.

Briefly, 10⁶ CT26-Tyrp1 colon carcinoma cells were injected subcutaneously into the flank of Balb/c mice. On Day 14 after tumor inoculation, when tumor volume reached 270 mm³, the mice were randomized into different treatment groups and intraperitoneally injected with 1 μg of DF-mIL-12-Fc si at a molar dose equivalent to 1 μg rmIL-12 or 1 μg of mIgG2a isotype control once a week. Tumor growth was assessed for 60 days. The complete responders were re-challenged with 10⁶ CT26 cells 72 days after first tumor inoculation. Age-matched naïve Balb/C mice were used as control group.

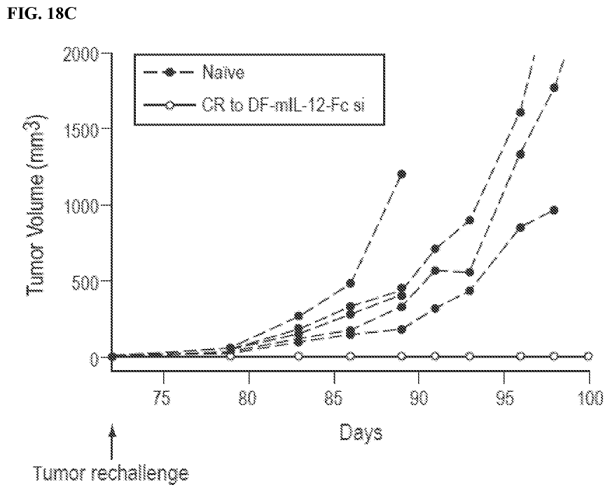

FIG. 18A is a graph showing tumor growth curves of individual mice inoculated with CT26 tumor cells and administered a single dose of 1 μg of DF-mIL-12-Fc si or mIgG2a isotype. FIG. 18B is a graph showing body weights of individual mice inoculated with CT26 tumor cells and administered a weekly dose of 1 μg of DF-mIL-12-Fc si or mIgG2a isotype. FIG. 18C is a graph showing tumor growth curves of individual mice re-challenged with inoculation of CT26 tumor cells.

As shown in FIGS. 18A-B, administration of DF-mIL-12-Fc si resulted in robust tumor regression in comparison to mIgG2a isotype with no observable toxicity affecting the body weight of treatment animals. As shown in FIG. 18C, the initial DF-mIL-12-Fc si treated mice remained tumor-free suggesting the formation of immunological memory with DF-mIL-12-Fc si treatment.

### Example 10—DF mIL-12-Fc Si Delivered Intraperitoneally or Subcutaneously is Effective to Reduce Tumor Volume in CT26 Tumor Model

This example shows that intraperitoneal or subcutaneous administration of DF-mIL-12-Fc si ensures 100% complete recovery in mice bearing CT26 tumors.

Briefly, 10⁶ CT26-Tyrp1 colon carcinoma cells were injected subcutaneously into the flank of Balb/c mice. On Day 14 after tumor inoculation, when tumor volume reached 270 mm³, the mice were randomized into different treatment groups and intraperitoneally injected with 1 μg of DF-mIL-12-Fc si at a molar dose equivalent to 1 μg IL-12 or 1 μg of mIgG2a isotype control once a week, or subcutaneously injected with 1 μg of DF-mIL-12-Fc si at a molar dose equivalent to 1 μg IL-12 or 1 μg of mIgG2a isotype control once a week. Tumor growth was assessed for more than 60 days.

FIG. 19A is a graph showing tumor growth curve of individual mice inoculated with CT26 tumor cells and administered a weekly dose of 1 μg of DF-mIL-12-Fc si or mIgG2a isotype delivered intraperitoneally. FIG. 19B is a graph showing tumor growth curve of individual mice inoculated with CT26 tumor cells and administered a weekly dose of 1 μg of DF-mIL-12-Fc si or mIgG2a isotype delivered subcutaneously.

As shown in FIGS. 19A-B, either intraperitoneal or subcutaneous delivery of DF-mIL-12-Fc si was effective at reducing CT26 tumor volume.

### Example 11—Single Dose Administration of DF-mIL-12-Fc Si is Effective to Reduce Tumor Volume in B16F10 Mouse Model

This example shows that a single dose of DF-m112-Fc si is effective at reducing tumor volume in mice bearing B16F10 melanoma tumors.

In brief, C57BL/6 mice were injected with 10⁶ B16F10 melanoma cells subcutaneously into the flank of the mice. On Day 7 after tumor inoculation mice were randomized. When tumor average reached 200 mm³, mice were treated intraperitoneally with a single dose of isotype control, or 1 μg of DF-mIL-12-Fc si. Tumor growth was assessed for 50 days.

As shown in FIG. 20, a single administration of μg of DF-mIL-12-Fc si is effective to reduce tumor volume in B16F10 tumor-bearing mice.

### Example 12—DF-mIL-12-Fc Si Delivered Intraperitoneally or Subcutaneously is Effective to Reduce Tumor Volume in B16F10 Mouse Model

This example shows that intraperitoneal or subcutaneous administration of DF-mIL-12-Fc si led to 100% complete recovery in mice bearing B16F10 melanoma tumors.

Briefly, 10⁶ B16F10 melanoma cells were injected subcutaneously into the flank of C57BL/6 mice. On Day 7 after tumor inoculation, mice were randomized. When tumor average reached 200 mm³, mice were intraperitoneally injected with 1 μg of DF-mIL-12-Fc si at a molar dose equivalent to 1 μg IL-12 or 1 μg of mIgG2a isotype control once a week, or subcutaneously injected with 1 μg of DF-mIL-12-Fc si at a molar dose equivalent to 1 μg IL-12 or 1 μg of mIgG2a isotype control once a week. Tumor growth was assessed for 40 days.

As shown in FIGS. 21A-B, either intraperitoneal or subcutaneous delivery of DF-mIL-12-Fc si was effective at reducing B16F10 tumor volume as compared to isotype control.

### Example 13—DF-mIL-12-Fc Si is Efficacious as a Single Dose

This example shows that DF-mIL-12-Fc si is effective at reducing CT26 tumor volume when administered as a single dose, and when administered via repeat dosing, is even more effective.

Briefly, 10⁶ CT26-Tyrp1 colon carcinoma cells were injected subcutaneously into the flank of Balb/c mice. On Day 14 after tumor inoculation, when tumor volume reached 270 mm³, the mice were randomized into different treatment groups (n=10 per group) and intraperitoneally injected with a single dose of 1 μg of DF-mIL-12-Fc si at a molar dose equivalent to 0.1 μg IL-12 or 1 μg of mIgG2a isotype control once a week. Alternatively, mice were intraperitoneally injected with 1 μg of DF-mIL-12-Fc si at a molar dose equivalent to 1 μg IL-12 or 1 μg of mIgG2a isotype control once a week. Tumor growth was assessed for more than 60 days.

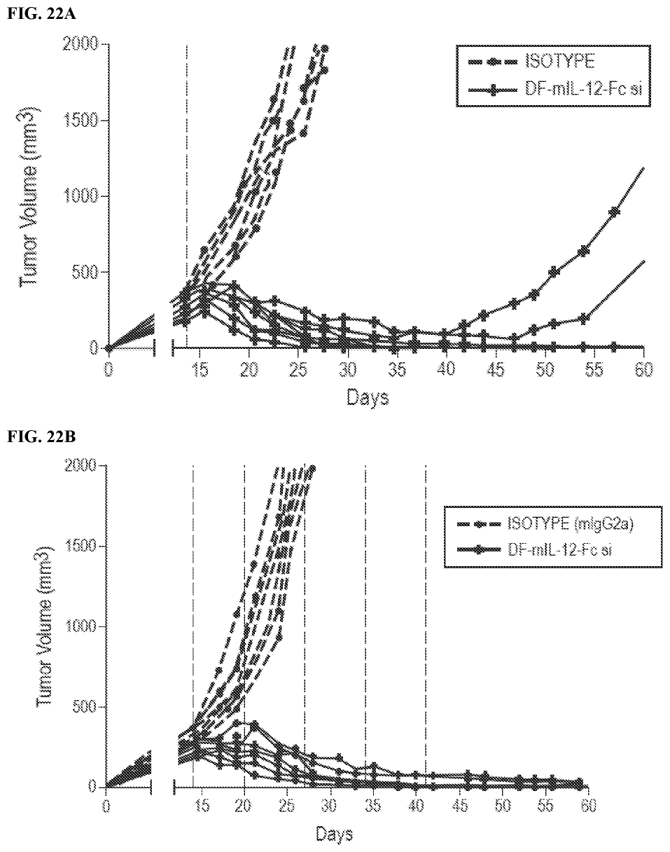

FIG. 22A is a graph showing tumor growth curve of individual mice inoculated with CT26 tumor cells and administered a single dose of 1 μg of DF-mIL-12-Fc si or mIgG2a isotype. FIG. 22B is a graph showing tumor growth curve of individual mice inoculated with CT26 tumor cells and administered a weekly dose of 1 μg of DF-mIL-12-Fc si or mIgG2a isotype.

As shown in FIG. 18A and FIG. 22A, a single administration of 1 μg of DF-mIL-12-Fc si resulted in robust 70% complete recovery of tumor-bearing mice as compared to mIgG2a isotype. However, as shown in FIG. 2C and FIG. 22B, repeat weekly dosing of 1 μg of DF-mIL-12-Fc si ensured 100% complete recovery of tumor-bearing mice as compared to mIgG2a isotype. As shown in FIG. 18B, even repeat administration of DF-mIL-12-Fc si was well-tolerated with no toxicities observed, as assessed by body weight.

Additionally, complete responders (CR) were re-challenged with 5×10⁵ CT26-Tyrp1 colon carcinoma cells at the opposite flank and tumor progression was compared to naïve mice challenged at the same tumor dose. As shown in FIG. 18C, while tumors grew in naïve mice, 100% of complete responders remained tumor-free following re-challenge. Accordingly, a single administration of DF-mIL-12-Fc si demonstrated a complete, durable response in a population of mice.

### Example 14—Pharmacokinetics in Cynomolgus Monkeys Treated with a Single Subcutaneous Dose of DF-hIL-12-Fc Si

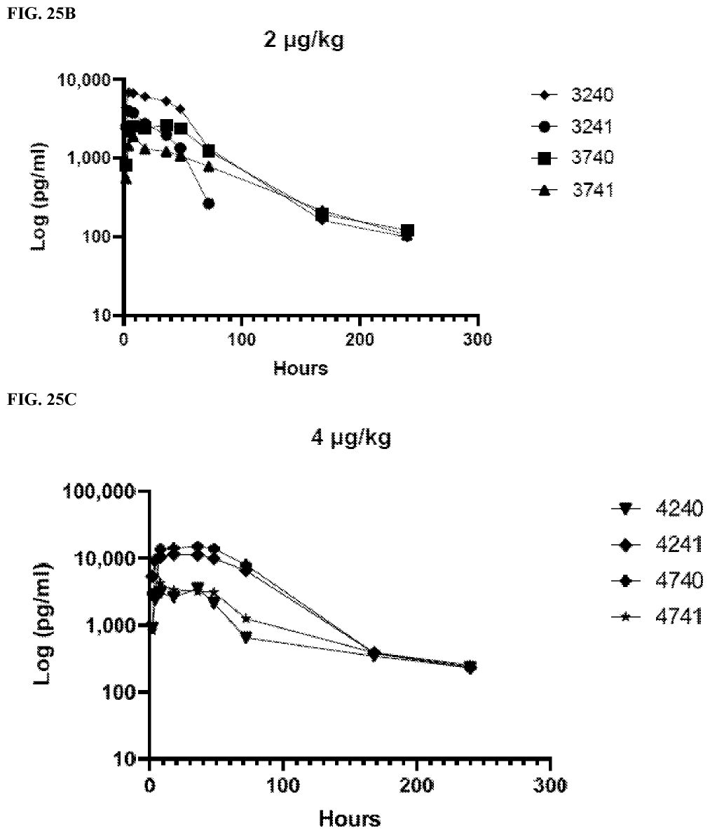

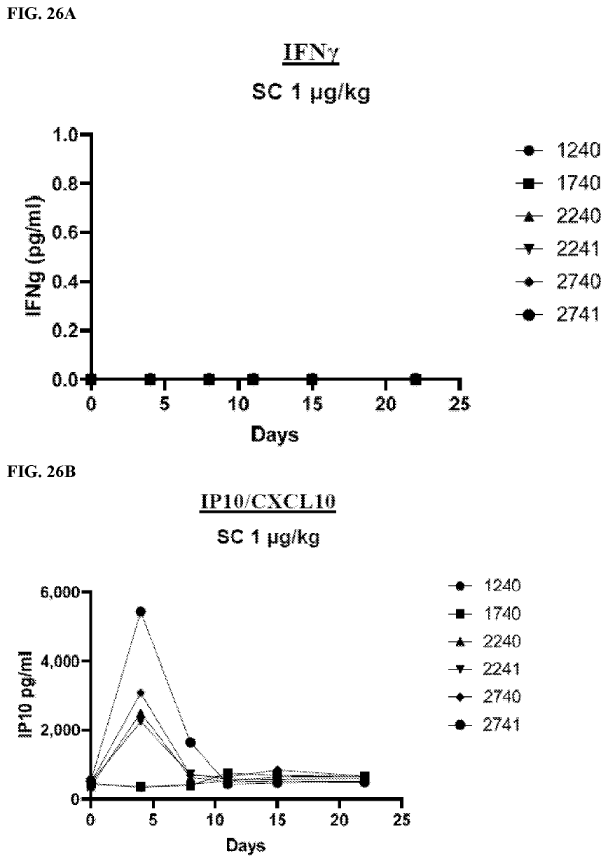

Pharmacokinetics were determined following a subcutaneous injection of DF-hIL-12-Fc si at 1 μg/kg (FIG. 25A), 2 μg/kg (FIG. 25B), or 4 μg/kg (FIG. 25C) in cynomolgus monkeys utilizing an ELISA like immunoassay-Meso Scale Discovery (MSD) immunoassay method. Briefly, an untreated MSD microtiter plate was coated with monkey-adsorbed goat anti-human IgG and incubated at room temperature. Following coating and incubation, the plate was washed, blocked, washed, and incubated with standard curve and quality control samples spiked with a DF-hIL-12-Fc si reference standard, along with test samples. Following incubation, the plate was washed and biotin anti-human IL-12/IL-23 p40 was added to the plate as the primary detection antibody. Following another wash step, streptavidin-conjugated Sulfo-Tag was added as the secondary detection antibody. The plate was washed a final time before adding MSD read buffer T to the plate. The plate was read using an MSD Sector Imager S6000.

FIGS. 25A-25C are line graphs showing pharmacokinetics in cynomolgus monkeys treated with a single subcutaneous dose of 1 μg/kg (FIG. 25A), 2 μg/kg (FIG. 25B), or 4 μg/kg (FIG. 25C) of DF-hIL-12-Fc si.

The data indicate that concentrations of DF-hIL-12-Fc si and rhIL-12 decreased over time, as expected, with similar pharmacokinetic profiles at all doses tested.

### Example 15—Cytokine Release in Cynomolgus Monkeys Treated with a Single Subcutaneous Dose of DF-hIL-12-Fc Si

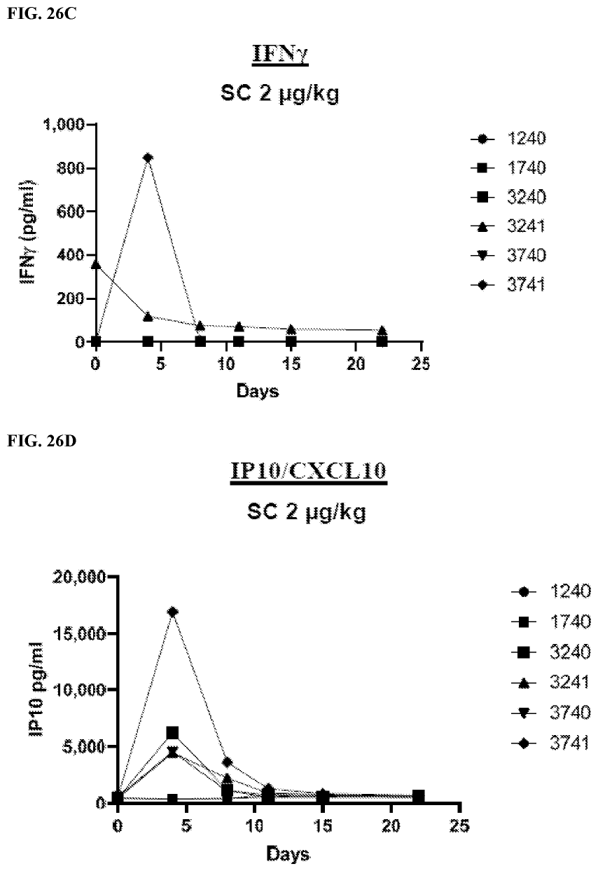

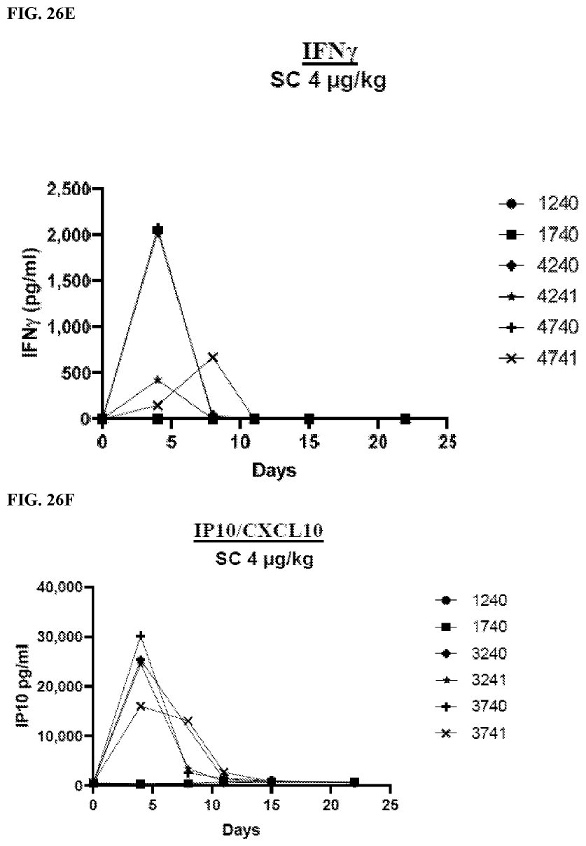

Quantitative measurements of cytokines following a subcutaneous injection of DF-hIL-12-Fc si at 1 μg/kg (FIGS. 26A and 26B), 2 μg/kg (FIGS. 26C and 26D), or 4 μg/kg (FIGS. 26E and 26F) in cynomolgus monkeys were determined using MSD immunoassay kits. The method used sandwich immunoassay kits (Pro-inflammatory Panel 1 Biomarkers and V-PLEX Plus Chemokine Panel 1 NHP Kit) for the relative quantitative measurement of Pro-inflammatory Panel 1 Biomarkers: IFNγ, IL-1β, IL-2, IL-6 IL-8, and IL-10 in cynomolgus monkey K2 EDTA plasma (referred to as monkey plasma). The method is based on MSD non-human primate (NHP) kits for V-PLEX and V-PLEX Plus, Catalog No. K15056D-1, K15056D-2, K15056D-4, K15056D-6, K15056G-1, K15056G-2, K15056G-4, K15056G-6. The method employs human capture and detection antibodies that react with cynomolgus monkeys. The kit provides plates pre-coated with capture antibodies on independent, well-defined spots within each well of a 96-well multi-spot plate. The plate was incubated with monkey plasma samples, washed and then incubated with detection antibodies (specific for each analyte) that are conjugated with electrochemiluminescent (ECL) labels (MSD SULFO-TAG). Analytes in the sample bind to capture antibodies immobilized on the working electrode surface; recruitment of the detection antibodies by the bound analytes completes the sandwich. The plate was washed and an MSD Read Buffer was added to create the appropriate chemical environment for electrochemiluminiscence (ECL). The plate was loaded into an MSD Sector Imager 600 (SI600) instrument where a voltage was applied to the plate electrodes causing the captured labels to emit light. The instrument measures the intensity of emitted light in terms of Relative Light Units (RLU) to provide a relative quantitative measure of analytes in the sample. Raw RLU data was exported into a text file, which then was converted into a Watson LIMS compatible file using a programmed Excel spread sheet, which was custom designed at Envigo. Data was subsequently imported and regressed in Watson LIMS Software v.7.2.0.02.

FIGS. 26A-26F are line graphs showing concentrations of IFNγ (FIGS. 26A, 26C, and 26E) and IP10/CXCL10 (FIGS. 26B, 26D, and 26F) in cynomolgus monkeys treated with a single subcutaneous dose of 1 μg/kg (FIGS. 29A and 29B), 2 μg/kg (FIGS. 26C and 26D), or 4 μg/kg (FIGS. 26E and 26F) of DF-hIL-12-Fc si.

As shown in FIG. 26A, a single subcutaneous dose of DF-hIL-12-Fc si at 1 μg/kg did not result in detectable levels of IFNγ. Subcutaneous doses of DF-hIL-12-Fc si at 2 μg/kg and 4 μg/kg, resulted in an increase in IFNγ levels in some animals that peaked at day 4 post-dosing (FIGS. 26C and 26E). Subcutaneous doses of DF-hIL-12-Fc si at 1 μg/kg, 2 μg/kg, and 4 μg/kg all resulted in elevated IP10/CXCL10 levels that peaked at day 4 post-dosing (FIGS. 26B, 26D, and 26F).

### Example 16—DF-mIL-12-Fc Si Combination Therapy Using Radiation or Chemotherapy in 4T1 Orthotopic Mouse Model

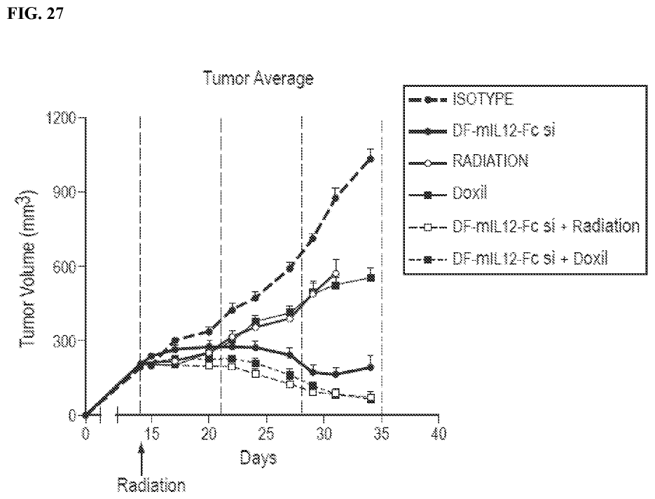

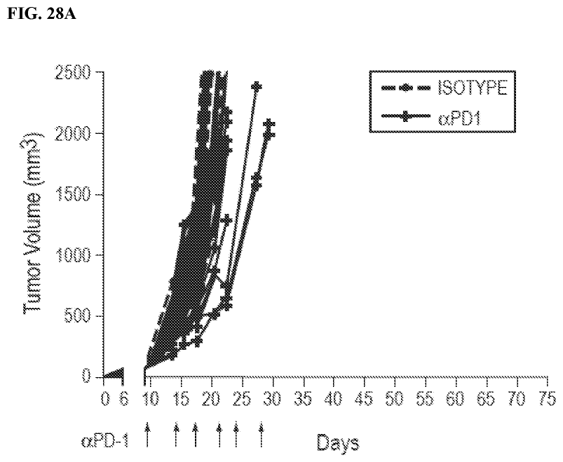

In order to show whether anti-tumor activity elicited by administration of DF-mIL-12-Fc si can be amplified, combination studies using radiation or chemotherapy were performed. Briefly, Balb/c mice were injected orthotopically into the mammary fat pad with 5×10⁵ 4T1-luc tumor cells. On Day 14 after tumor inoculation, mice were randomized (n=10 per group). Mice were treated subcutaneously with either isotype, DF-mIL-12-Fc si (both equimolar to 1 μg IL-12), 5 mg/kg Doxil® (chemotherapy) intravenously, or irradiated with 10 Gy as monotherapy, or DF-mIL-12-Fc si in combination with Doxil® or radiation. Tumor growth was assessed over time. FIG. 27 is a graph showing tumor growth curves of individual mice inoculated with breast cancer cells and administered a weekly dose of isotype control, DF-mIL-12-Fc si, Doxil (chemotherapy), or irradiated with 10 Gy as monotherapy or DF-mIL-12-Fc si in combination with Doxil® or radiation. Graph shows group averages of tumor growth±standard error mean.

As seen in FIG. 27, although monotherapy with DF-mIL-12-Fc si was effective by itself in 4T1 tumor-bearing mice, combination therapy amplified anti-tumor immune responses leading to full tumor regression in 10-30% of mice.

### Example 17—DF-mIL-12-Fc Si Mediated Anti-Tumor Efficacy Against Large, PD-1 Blockade-Resistant CT26 Colon Carcinoma Tumors

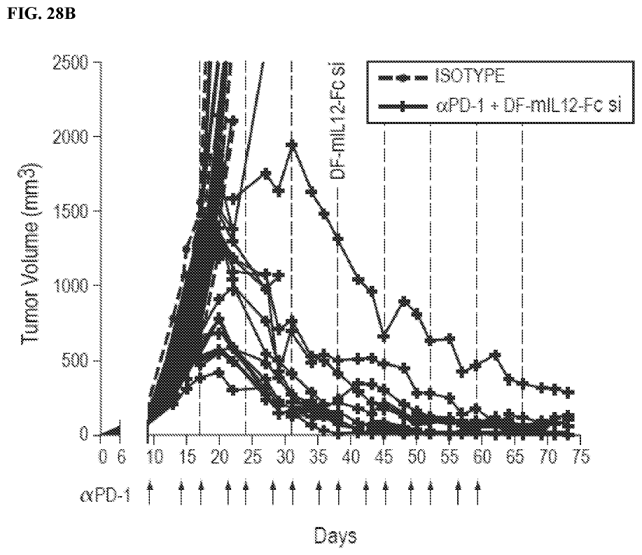

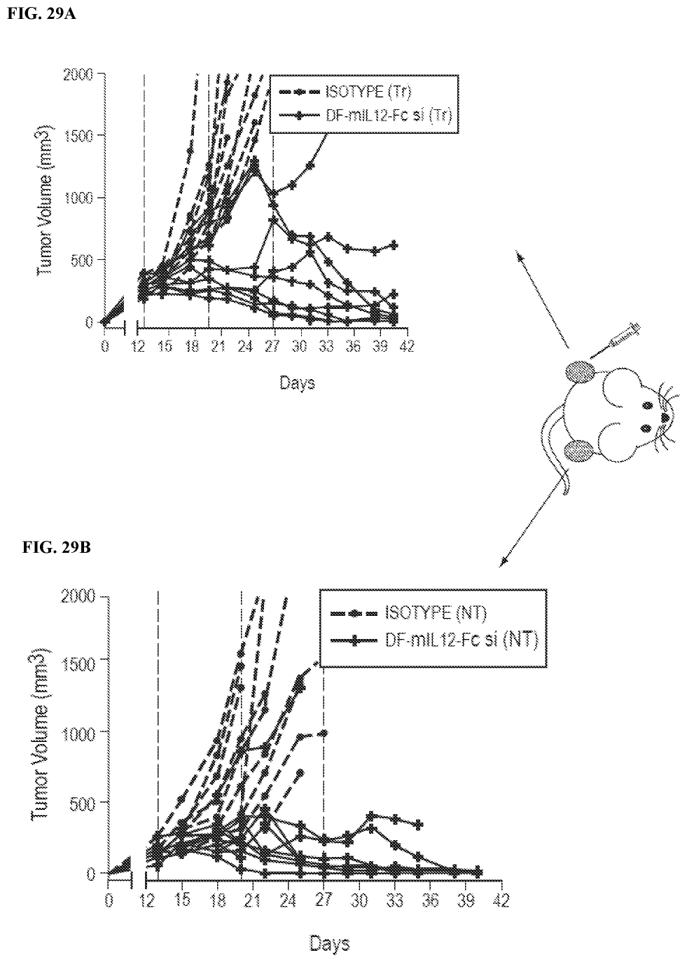

This example analyzes whether DF-mIL-12-Fc si elicited potent, anti-tumor responses against PD-1 blockade-resistant CT26-Tyrp1 tumors. Briefly, Balb/c mice were injected with 0.5×10⁶ CT26-Tyrp1 tumor cells. Following inoculation when average tumor volume reached ˜120 mm³, mice were randomized on Day 9. Mice were either treated with 200 μg isotype or anti-PD-1 antibody (twice weekly). FIG. 28A is a graph showing tumor growth curve of Balb/c mice inoculated with CT26-Tyrp1 tumor cells and treated (bi-weekly) either with isotype control or anti-PD-1 antibody. On day 17, the group previously treated with anti-PD-1 (with an average tumor volume ˜800 mm³) was subdivided into two treatment groups. Group 1 continued to receive PD-1 blockade treatment twice weekly, Group 2 received PD-1 blockade (twice weekly) along with DF-mIL-12-Fc si (1 μg weekly). FIG. 28B is a graph showing tumor growth curve of the previously anti-PD-1 antibody-treated Balb/c mice treated with anti-PD-1 antibody (bi-weekly) along with weekly treatment with 1 μg of DF-mIL-12-Fc si.

As shown in FIG. 28A, anti-PD-1 monotherapy failed to control tumor progression. However, as shown in FIG. 28B, the addition of DF-mIL-12-Fc si resulted in effective tumor regression.

### Example 18—Local Treatment of DF-mIL-12-Fc Si Against Large CT26 Colon Carcinoma Tumors Induces Abscopal Anti-Tumor Responses

This example shows whether DF-mIL-12-Fc si treatment can induce abscopal therapeutic effects. Briefly, Balb/c were implanted subcutaneously with CT26-Tyrp1 colon carcinoma cells on both the left (0.8×10⁶ tumor cells) and right (0.4×10⁶ tumor cells) flank. On Day 13 after tumor inoculation, left tumors were injected either with 0.1 μg isotype control or 0.1 μg DF-mIL-12-Fc si once weekly for 2-3 weeks. FIG. 29A is a graph showing tumor growth curve of the treated (Tr) tumor in Balb/c mice inoculated with CT26-Tyrp1 tumor cells and treated once (weekly) with either isotype control or DF-mIL-12-Fc si. Right tumors were left untreated (NT).

FIG. 29B is a graph showing tumor growth curves of the untreated (NT) tumors in Balb/c mice inoculated with CT26-Tyrp1 tumor cells.

As shown in FIGS. 29A-29B, control isotype-treated tumors grew progressively at both right and left sites. As shown in FIGS. 29A-29B, DF-mIL-12-Fc si caused effective anti-tumor responses at the local injected site (FIG. 29A) and the distant non-treated tumor (FIG. 29B) indicating abscopal therapeutic effects.

### Example 19—DF-mIL-12-Fc Si Mediated Anti-Tumor Efficacy Against Large CT26 Colon Carcinoma Tumors

This example shows that DF-mIL-12-Fc si which includes wild-type murine IL-12 p40 and p35 subunits fused to the N-termini of murine IgG2a Fc domain polypeptides with mutations L234A, L235A, and P329G (discussed in Example 2) is efficacious against larger tumor volumes.

DF-mIL-12-Fc si-mediated anti-tumor efficacy against large CT26 colon carcinoma tumors was tested. Briefly, Balb/c mice were injected subcutaneously with 10⁶ CT26-Tyrp1 colon carcinoma cells. On Day 18 after tumor inoculation, when tumor volume reached 800 mm³, the mice were randomized into different treatment groups (n=10 per group) and treated intraperitoneally with DF-mIL-12-Fc si at a molar dose equivalent to 1 μg or 2 μg IL-12, or molar equivalent of mIgG2a isotype once or once weekly.

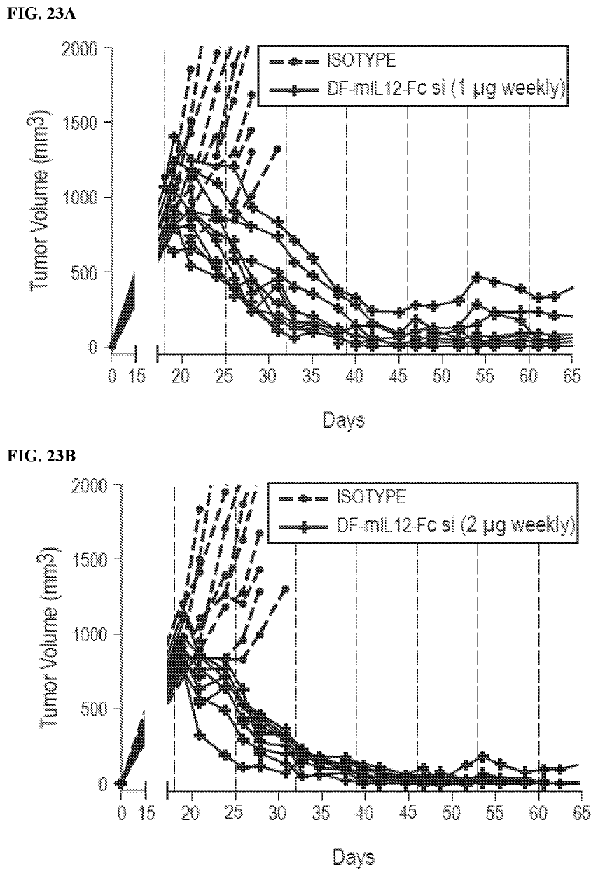

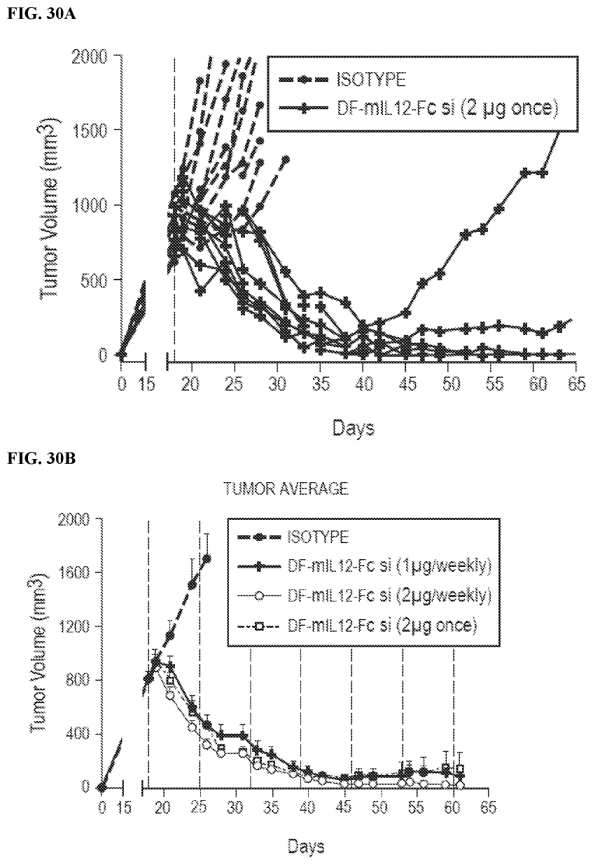

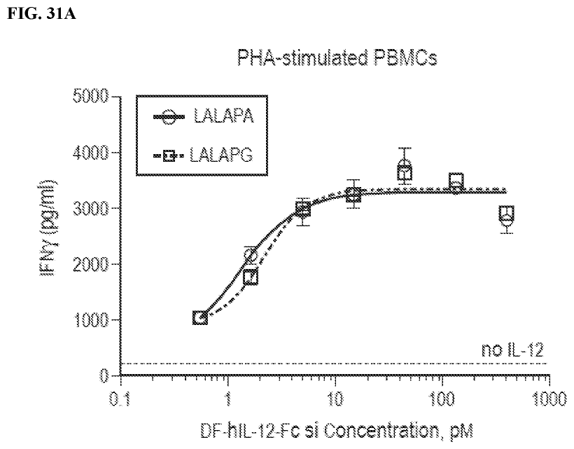

Tumor growth was assessed for 65 days. FIG. 23A is a graph showing tumor growth curves of Balb/c mice inoculated with CT26-Tyrp1 tumor cells and treated once (weekly) with either 2 μg mIgG2a isotype control or 1 μg DF-mIL-12-Fc si. FIG. 23B is a graph showing tumor growth curves of Balb/c mice inoculated with CT26-Tyrp1 tumor cells and treated once (weekly) with either 2 μg mIgG2a isotype control or 2 μg DF-mIL-12-Fc si. FIG. 30A is a graph showing tumor growth curves of Balb/c mice inoculated with CT26-Tyrp1 tumor cells and treated once with either 2 μg mIgG2a isotype control or 2 μg DF-mIL-12-Fc si. FIG. 30B is a graph showing average tumor growth curves of Balb/c mice inoculated with CT26-Tyrp1 tumor cells and treated with 2 μg mIgG2a isotype control, 1 μg DF-mIL-12-Fc si (weekly administration), 2 μg DF-mIL-12-Fc si (weekly administration), or 2 μg DF-mIL-12-Fc si (once). FIGS. 23A, 23B, and 30A show tumor growth curves of individual mice. FIG. 30B shows tumor average±standard error mean.

As shown in FIGS. 23A, 23B, and 30B, weekly doses (1 μg or 2 μg) of DF-mIL-12-Fc si were efficient in controlling tumor progression and 100% of mice responded to DF-mIL-12-Fc si treatment. Additionally, as shown in FIG. 30A, a single treatment with 2 μg DF-mIL-12-Fc si showed tumor regression yielding a 100% response rate. The data and figures described in this example show that DF-mIL-12-Fc si is not only effective at reducing larger CT26 tumor volume but also effective at reducing CT26 tumor volume when administered as a single dose.

### Example 20—DF-mIL-12-Fc Si Treatment Against B16F10 Melanomas Induces Production of Cytokines and Chemokines in Serum and in Tumors

This example shows that DF-mIL-12-Fc si treatment results in elevated levels of IFNγ, CXCL9, and CXCL10 in blood and tunors of C57BL/6 mice bearing B16F10 tumors. Briefly, C57BL/6 mice were injected subcutaneously with 10⁶ B16F10 melanoma cells. On Day 7 after tumor inoculation (when average tumor volume reached 150 mm³), mice were randomized (n=8 per group). Mice were treated intraperitoneally with isotype control, IL-12, or DF-mIL-12-Fc equimolar to 1 μg IL-12.

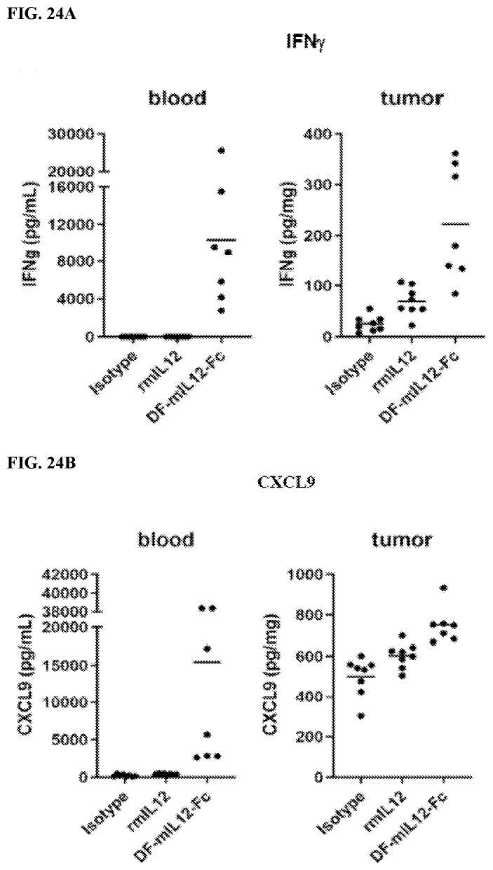

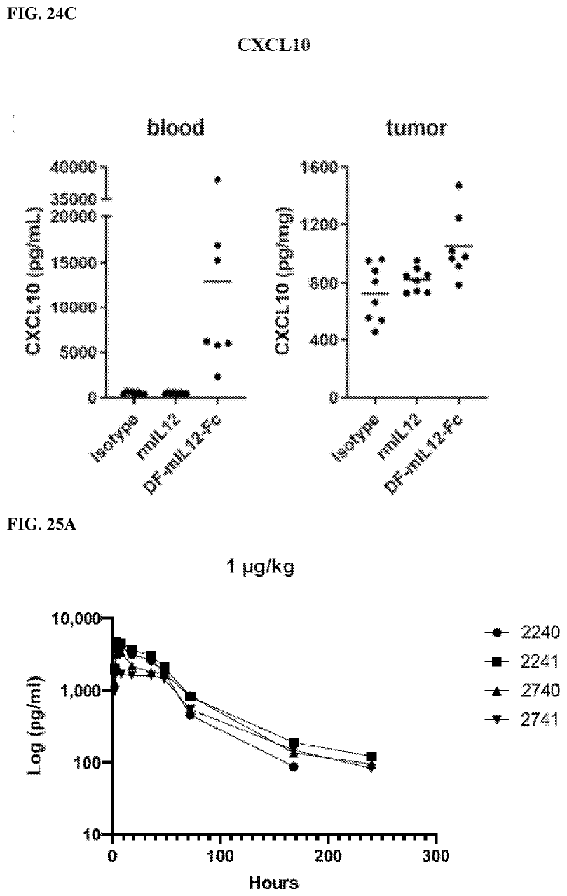

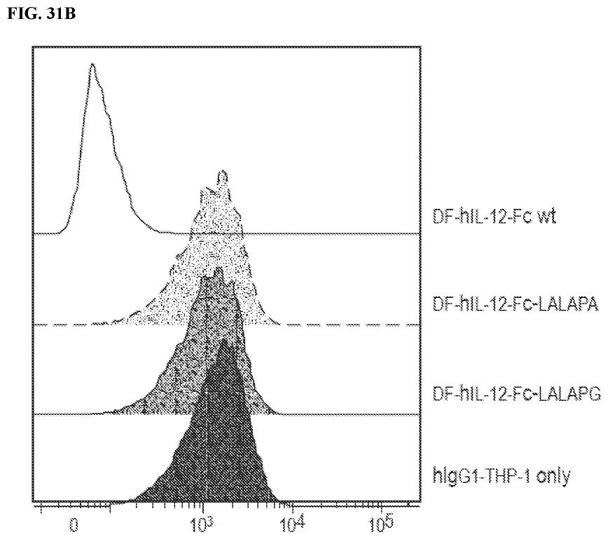

After 72 hours post-treatment, serum and tumor lysates were prepared and analyzed for IFNγ (FIG. 24A), CXCL9 (FIG. 24B), and CXCL10 (FIG. 24C) expression using multiplex technology. FIGS. 31A-C show average cytokine/chemokine levels in mice.

As shown in FIGS. 24A-24C, a single administration of 0.5 μg of DF-mIL-12-Fc si resulted in increased expression of IFNγ (FIG. 24A), CXCL9 (FIG. 24B), and CXCL10 (FIG. 24C) in serum (left panel) and within tumors (right panel), whereas IL-12 treatment had little or no effect.

### Example 21—DF hIL-12-Fc Si Having LALAPA and LALAPG Mutations have Similar IFNγ-Stimulating Activity and Abrogated FcγR Binding

This example shows the IFNγ-stimulating and FcγR-binding activities of DF hIL-12-Fc si with IgG1 Fc having LALAPA (L234A, L235A, and P329A) mutations, or LALAPG (L234A, L235A, and P329G) mutations. In brief, human PBMCs were cultured for 2 days with both 5 μg/ml phytohemagglutinin (PHA) and a dose-titration of DF hIL-12-Fc-si, having LALAPA or LALAPG mutations. After 2-day stimulation, supernatants were harvested and IFNγ content measured by ELISA. For determining FcγR-binding activities, fluorophore-conjugated hIgG1 isotype antibody (83 nM) bound to THP-1 cells that express high affinity FcγRs CD32 and CD64 was detected by flow cytometry.

As shown in FIG. 31A, hIL-12-Fc-LALAPA and hIL-12-Fc-LALAPG have similar abilities to stimulate IFNγ production from PBMCs concurrently with PHA, well above the amount produced with PHA alone.

As shown in FIG. 31B, Simultaneous inclusion of 16-fold molar excess of hIL-12-Fc-wt (1.3 μM) in a mixture with the labeled hIgG1 isotype antibody resulted in substantial reduction of binding signal, likely due to competition for IgG1 binding to CD32 and CD64. In contrast, at the same concentration, neither incubation with hIL-12-Fc-LALAPA nor hIL-12-Fc-LALAPG resulted in detectable IgG1 isotype binding, suggesting abrogated FcγR engagement for both proteins attributable to the LALAPA and LALAPG mutations.

### Example 22: Manufacturing Process and Process Controls for DF hIL12-Fc Si

DF hIL12-Fc si is expressed in Chinese Hamster Ovary (CHO) cells in a suspension culture. Cells from the Master Cell Bank (MCB) are used to inoculate shake flasks containing chemically defined medium free of animal components. The cells are then used to inoculate progressively larger volume cultures to expand the cell number to enable inoculation of the production bioreactor.

The production bioreactor is operated in fed-batch mode to increase expression of the DF hIL12-Fc si protein. After approximately 14 days, the culture is harvested by depth filtration to remove cells and cell debris prior to initial purification. DF hIL12-Fc si is purified from the CHO harvest medium using a series of chromatography and filtration steps, including Protein A capture chromatography, Mixed Mode chromatography and Cation exchange chromatography (CEX).

Two dedicated, orthogonal viral inactivation and removal steps are included—low pH inactivation and nanofiltration. The viral inactivation step included addition of acetate to the filtered DF hIL12-Fc si solution to adjust the pH to about 3.65, and incubating for at least 60 minutes.

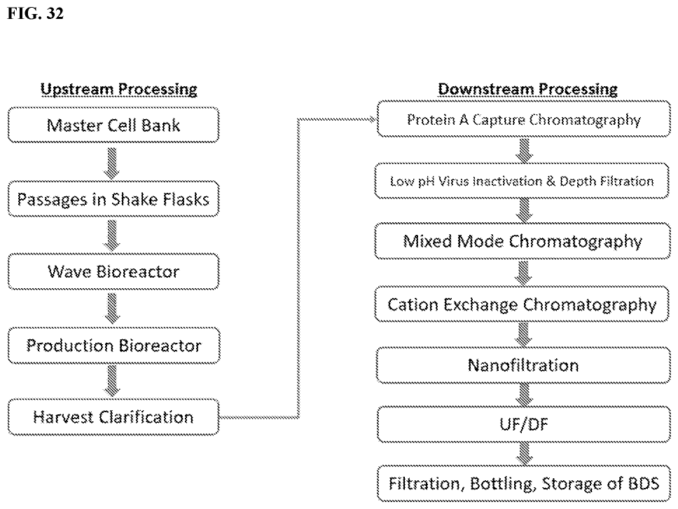

Finally, DF hIL12-Fc si is concentrated and formulated in a final composition of 20 mM Citrate, 6% Sucrose, 1% Mannitol and 0.01% (w/v) polysorbate 80. The formulated drug substance is then filtered through a 0.2 μm membrane into polycarbonate bottles prior to storage at ≤−65° C. A schematic of the entire DF hIL12-Fc si drug substance manufacturing process is provided in FIG. 32.

### Batch Scale and Definition

A single vial of DF hIL12-Fc si MCB is expanded to one production bioreactor and each harvest is purified into one lot of drug substance.

### Cell Culture and Upstream Manufacturing Process

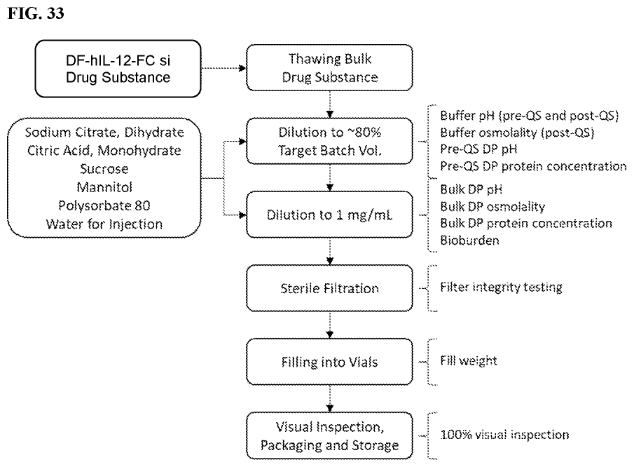

The upstream drug substance manufacturing process for DF hIL12-Fc si is shown in FIG. 33 and additional details for each unit operation are provided.

### Shake Flask Passages

A vial of the master cell bank is thawed in a 37° C. water bath and the contents are slowly mixed by pipette and then added to a 125 mL shake flask containing pre-equilibrated growth medium (BalanCD CHO Growth Medium A, Irvine Scientific) supplemented with 6 mM L-glutamine. A cell count is taken after inoculation, and if necessary, the cell density is diluted to a target of 0.30×10⁶ to 0.50×10⁶ viable cells/mL. The flask is then placed on an orbital shaker in an incubator with temperature and % C02 (g) control. Cell density and viability are checked on day 3 prior to Passage 2.

For Passage 2, a 500 mL shake flask is pre-equilibrated with growth medium supplemented with 6 mM L-glutamine. The flask is then inoculated with cells from Passage 1 and placed on an orbital shaker in an incubator with temperature and % CO₂ (g) control. Cell density and viability are measured, and the cells are used to inoculate Passage 3 once the forward processing criteria are met.

For Passage 3, three 1000 mL shake flasks are pre-equilibrated with growth medium supplemented with 6 mM L-glutamine. The flasks are then inoculated with cells from Passage 2 and then placed on an orbital shaker in an incubator with temperature and % CO₂ (g) control. Cell density and viability are measured, and the cells are used to inoculate Passage 4 once the forward processing criteria are met.

For Passage 4, four 5000 mL shake flasks are pre-equilibrated with growth medium supplemented with 6 mM L-glutamine. The flasks are then inoculated with cells from Passage 2 and then placed on an orbital shaker in an incubator with temperature and % CO₂ (g) control. Cell density and viability are measured, and the cells are used to inoculate the 50 L Wave bioreactor once the forward processing criteria are met. Process parameter ranges and in-process tests are summarized in Table 17.

TABLE 17
Shake Flask Passages-Process Parameters and In-Process Tests

| Unit Operation | Parameter Setpoints/Ranges | In-Process Tests |
|---|---|---|
| Passage #1: | 30 mL working volume; 36.5° C. | Viable cell density |
| 125 mL shake flask | 5% CO₂ (g); 120 rpm | Viability |
| Passage #2: | 150 mL working volume; 36.5° C. | Viable cell density |
| 500 mL shake flask | 5% CO₂ (g); 120 rpm | Viability |
| Passage #3: | 400 mL working volume; 36.5° C. | Viable cell density |
| 3 × 1000 mL shake flasks | 5% CO₂ (g); 120 rpm | Viability |
| Passage #4: | 1500 mL working volume; 36.5° C. | Viable cell density |
| 4 × 5000 mL shake flasks | 5% CO₂ (g); 120 rpm | Viability |

### Wave Bioreactor

A 50 L Wave Bioreactor™ platform (GE Healthcare LifeSciences) is setup and inoculated with growth medium (BalanCD CHO Growth Medium A, Irvine Scientific) supplemented with 6 mM L-glutamine. The media is pre-conditioned at 36.5° C. and 5% CO₂ (g) and then inoculated with culture from Passage 4. The bioreactor is sampled daily for cell density and viability, and the culture is used to inoculate the 200 L production bioreactor once the transfer cell density criteria is achieved. Metabolite concentrations (e.g, glucose and lactate) and pH are also monitored on a daily basis for information. Process parameter ranges and in-process tests are summarized in Table 18.

TABLE 18
Wave Bioreactor-Process Parameters and In-Process Tests

| Unit Operation | Parameter Setpoints/Ranges | In-Process Tests |
|---|---|---|
| Wave | 20 L working volume; | Viable cell density |
| Bioreactor | 36.5° C.; 5% CO₂ (g) | Viability |

### Production Bioreactor

A 200 L disposable bioreactor is setup and inoculated with growth medium supplemented with 6 mM L-glutamine. The media is pre-equilibrated at 37° C. and then inoculated with culture from the 50 L Wave Bioreactor. Initial inoculation volume is approximately 130 L and final culture volume is approximately 180 L. Dissolved oxygen is controlled with air and oxygen supplementation and pH is controlled with addition of carbon dioxide gas and/or sodium carbonate base. The production bioreactor is sampled daily for cell density and viability and once the viable cell density is ≥14×10⁶ viable cells/mL, the temperature setpoint is shifted from 37° C. to 33° C. and maintained at 33° C. until harvest criteria is met. The culture is harvested when the viability is ≤85% viability or day 14 of culture, whichever comes first. Metabolite concentrations (e.g., glucose and lactate) and DF hIL12-Fc si titer (starting on day 8) are monitored during the culture period.

Starting on day 3 of culture, concentrated nutrient feeds are added on a daily basis until day 13. In addition, a concentrated glucose solution is added as needed to maintain a minimum concentration of glucose in the bioreactor after feeding. Beginning on day 3, antifoam is added to the bioreactor each day to minimize foam build-up. On the day of harvest, samples of the bioreactor culture are taken for adventitious agent testing. Process parameter ranges and in-process tests are summarized in Table 19.

TABLE 19
Production Bioreactor-Process Parameters and In-Process Tests

| Unit Operation | Parameter Setpoints/Ranges | In-Process Tests |
|---|---|---|
| Production | 130 L initial working volume | Viable cell density |
| Bioreaction | 36.5 ± 0.5° C. (prior to temp shift) | Viability |
| | 6.75 − 7.05 pH (prior to temp shift) | Mycoplasma |
| | 33.0 ± 0.5° C. (after temp shift) | Non-host contamination |
| | 6.80 − 7.20 pH (after temp shift) | Mouse minute virus by qPCR |
| | 40% dissolved oxygen | In vitro adventitious agents |
| | | Transmission electron microscopy |
| | | DF hIL12-Fc si titer |

### Harvest Clarification

The bioreactor is clarified by depth filtration to remove cells and cell debris in preparation for further purification steps. A two-stage single-use depth filtration system consisting of DOHC and XOHC filters is used for clarification. Prior to the start of filtration, the bioreactor temperature is adjusted to 18° C. and the dissolved oxygen setpoint is increased to 70% of saturation.

The harvest filters are rinsed with water for injection (WFI) and then equilibrated with buffer. The cell suspension is passed through the harvest filters using a peristaltic pump and the filters are flushed to collect the product. Pressure is monitored and maintained at ≤15 psig. The filtrate is then filtered through a 0.45/0.2 μm membrane into a sterile bag. Process parameter ranges and in-process tests are summarized in Table 20.

Clarified harvest is stored at 2-8° C. prior to the capture chromatography step unless processed immediately.

TABLE 20
Harvest Clarification-Process Parameters
and In-Process Tests

| | Parameter | |
|---|---|---|
| Unit Operation | Setpoints/Ranges | In-Process Tests |
| Clarification | ≤25 psig | DF hIL12-Fc si |
| | | concentration |
| | | by Protein A |
| | | HPLC Bioburden; |
| | | Endotoxin |

### Downstream Purification Manufacturing Process

The downstream drug substance manufacturing process for DF hIL12-Fc si is shown in FIG. 32 and additional details for each unit operation are provided in the text below. Downstream purification consists of three chromatography steps and two dedicated virus clearance steps, low pH inactivation and nanofiltration. For each process intermediate, hold-time studies have been performed to established allowed hold times and temperatures.

### Protein A Capture Chromatography

The clarified harvest is captured with Amsphere 3 Protein A (JSR Life Sciences) resin to remove process-related impurities (e.g., DNA and host cell proteins), media additives and serves as a volume reduction step prior to subsequent purification. Multiple cycles are performed for each lot as needed. Prior to each load, the resin is first equilibrated with 20 mM Tris, 150 mM NaCl, pH 7.5. Following loading, the column is washed with equilibration buffer to remove unbound or loosely bound impurities, and then a second wash with 50 mM acetate, pH 5.4 is performed to lower the pH and prepare the column for elution. DF hIL12-Fc si is eluted with 50 mM acetate, 100 mM arginine, pH 3.7 and collected by 280 nm UV wavelength starting at 1.25 AU/cm ascending and then ending at 1.25 AU/cm descending. The eluate is collected in one pool and each column cycle is individually processed by low pH virus inactivation. Process parameter ranges and in-process tests are summarized in Table 21.

TABLE 21
Protein A Capture Chromatography-Process
Parameters and In-Process Tests

| | Parameter Targets/ | In-Process |
|---|---|---|
| Unit Operation | Ranges | Tests |
| Protein A Capture | 20-33 g/L resin; | Bioburden |
| Chromatography | 15-25° C.; | Endotoxin |
| | Equilibration | |
| | pH: 7.5; Elution pH: 3.7; | |
| | Load/Wash/Elution | |
| | flow rate: 180 cm/hr | |

### Low pH Virus Inactivation

The protein A eluate is incubated at low pH to inactivate potentially present viruses. The pH of the capture eluate is adjusted with 0.5 M acetic acid as necessary and incubated for a minimum of 60 minutes. After the end of the incubation period, the inactivated pools are neutralized with 2 M Tris base and the material is passed through a 0.2 μm filtration assembly. Process parameter ranges and in-process tests are summarized in Table 22.

TABLE 22
Low pH Virus Inactivation-Process
Parameters and In-Process Tests

| | Parameter Targets/ | In-Process |
|---|---|---|
| Unit Operation | Ranges | Tests |
| Low pH viral | Acidification pH: | Bioburden |
| inactivation | 3.65 ± 0.05 pH | Endotoxin |
| | 60-75 minutes | |
| | Neutralization pH: | |
| | 5.2 ± 0.1; 15-25° C. | |

### X0SP Depth Filtration

The Virus Inactivated Neutralized (VIN) pool is processed through the X0SP intermediate depth filter to remove process related impurities (e.g., host cell proteins (HCP), host cell DNA). The system is flushed with WFI prior to loading DF hIL12-Fc si within the range of 500-1000 g/m². Following loading, the system is chased with 50 mM Acetate, pH 5.2 to complete product hold-up recovery. The X0SP pool conductivity is subsequently adjusted to ≤6.0 mS/cm with 50 mM Acetate pH 5.2 prior to loading onto the first chromatography step. Process parameter ranges and in-process tests are summarized in Table 23.

TABLE 23
X0SP Depth Filtration

| | Parameter Targets/ | In-Process |
|---|---|---|
| Unit Operation | Ranges | Tests |
| X0SP Depth | 500-1000 g/m2; ≤30 psig; | Bioburden |
| Filtration | 15-25° C. | Endotoxin |
| | Conductivity Adjustment: | |
| | ≤6.0 mS/cm | |

### Mixed Mode Chromatography

Mixed Mode chromatography by CaptoAdhere ImpRes (GE Healthcare) is performed in bind-elute mode to remove high molecular weight (HMW) species. The X0SP filtrate conductivity is adjusted to ≤6.0 mS/cm with 50 mM Acetate pH 5.2 as described above and split into multiple load cycles as needed. Prior to loading, the column is equilibrated with 50 mM Acetate pH 5.2 and loaded. After loading, the column is washed with 50 mM Acetate pH 5.2 and then eluted with 50 mM Acetate 250 mM NaCl pH 5.2. Collection is initiated by 280 nm UV detection at 0.625 AU/cm ascending and ended at 1.50 AU/cm descending. Following collection, each cycle is passed through a filter train containing a terminal 0.2 μm filter. Process parameter ranges and in-process tests are summarized in Table 24.

TABLE 24
Mixed Mode Chromatography-Process
Parameters and In-Process Tests

| | Parameter Targets/ | In-Process |
|---|---|---|
| Unit Operation | Ranges | Tests |
| Mixed Mode | Load conductivity: | Bioburden |
| Chromatography | 3.0-6.0 mS/cm; Load | Endotoxin |
| | pH: 5.1-5.3; Flow rate: | |
| | 150 cm/hr 15-23.5 g/L | |
| | resin load 15-25° C. | |

### Cation Exchange Chromatography

Cation exchange chromatography with Eshmuno CPX resin (EMD Millipore) is performed to remove product-related impurities (e.g., high molecular weight species, low molecular weight species), as well as additional process related impurity clearance. Multiple cycles are performed for each lot as needed. Prior to loading, the CaptoAdhere ImpRes cycles are pooled and dilute with 50 mM Tris, pH 7.4 buffer and pH adjusted to 7.50±0.20 with 2M Tris Base. The column is equilibrated with 50 mM Tris, pH 7.4 prior to loading the diluted and pH adjusted CaptoAdhere ImpRes pool. The column is then washed with 50 mM Tris, pH 7.4, and then eluted with a gradient of 50 mM Tris, pH 7.4 (Buffer A) and 50 mM Tris, 0.5 M NaCl, pH 7.4 (Buffer B).

Product collection is initiated by 280 nm UV detection starting at 2.5 AU/cm ascending and ending at 4.5 AU/cm descending. Following collection, each cycle is passed through a filter train containing a terminal 0.2 μm filter. Process parameter ranges and in-process tests are summarized in Table 25. PGP-51J1

TABLE 25
Eshmuno CPX-Process Parameters and In-Process Tests

| Unit Operation | Parameter Targets/Ranges | In-Process Tests |
|---|---|---|
| Cation Exchange | 10-14 g/L resin; Load pH: | Bioburden |
| | 7.50 ± 0.20; | |
| Chromatography | Load/Elution flow rate: | Endotoxin |
| | 200 cm/h 15-25° C. | |

### Nanofiltration

Nanofiltration is performed to remove any potentially present viruses based on size. The Eshmuno CPX eluate is passed through a prefilter (Viresolve Prefilter Pod, EMD Millipore) and then through a 20 nm nominal filter (Viresolve Pro Modus, EMD Millipore). Prior to loading, the system is flushed with WFI and equilibrated with 50 mM Tris, 265 mM NaCl, pH 7.4. After loading, the system is rinsed with equilibration buffer to recover the system hold-up. The filtrate is then passed through a 0.2 μm membrane prior to the next step. Process parameter ranges and in-process tests are summarized in Table 26.

TABLE 26
Nanofiltration-Process Parameters and In-Process Tests

| Unit Operation | Parameter Targets/Ranges | In-Process Tests |
|---|---|---|
| Nanofiltration | ≤500 L/m² load | Bioburden |
| | ≤30 psig | Endotoxin |
| | 15-25° C. | Filter integrity test |

### Ultrafiltration and Diafiltration (UF/DF)

Ultrafiltration and diafiltration are performed using Pellicon Ultracel D Screen regenerated cellulose 30 kDa molecular weight cut-off membranes. This step concentrates and exchange the DF hIL12-Fc si into the final formulation buffer at the intended concentration prior to final filtration and bottling. The system is first equilibrated with 50 mM Tris, 265 mM NaCl, pH 7.4 and then the viral filtrate pool is concentrated to a target of 5.0 g/L. Buffer exchange is then performed against a minimum of 7 diavolumes of 20 mM Citrate, pH 6.5. Following diafiltration, a second concentration is performed targeting 11.0 g/L and then the product is diluted to a final retentate target concentration of 7.5 g/L with diafiltration buffer.

A 20 mM Citrate, 18% (w/v) Sucrose, 3% (w/v) Mannitol, 0.03% (w/v) polysorbate-80, pH 6.5 stock solution is spiked into the UF/DF pool to target a final concentration of 20 mM Citrate, 6% (w/v) Sucrose, 1% (w/v) Mannitol, 0.01% (w/v) polysorbate-80 in the drug substance. Process parameter ranges and in-process tests are summarized in Table 27.

TABLE 27
UF/DF-Process Parameters and In-Process Tests

| Unit Operation | Parameter Targets/Ranges | In-Process Tests |
|---|---|---|
| Ultrafiltration | ≤250 g/m² load; | Bioburden |
| and | 12.0-18.0 psig | Endotoxin |
| Diafiltration | transmembrane pressure | DF hIL12-Fc si |
| | 7-9 diavolumes; 15-25° C. | Concentration |

### Filtration, Bottling, and Storage of Bulk Drug Substance

The formulated UF/DF retentate is filtered through a 0.2 μm membrane into the final drug substance storage containers, 2 L polycarbonate bottles with a polypropylene closures (Nalgene Biotainer). Filtration is performed in an ISO 5/Grade A area. Following filtration, each bottle is aseptically sampled, labeled and frozen at ≤−65° C. Process parameter ranges and in-process tests are summarized in Table 28.

TABLE 28
Filtration, Bottling, and BDS Storage-Process
Parameters and In-Process Tests

| Unit Operation | Parameter Targets/Ranges | In-Process Tests |
|---|---|---|
| Filtration, Bottling, | 1.0 L fill volume Storage | Filter integrity |
| and BDS Storage | at ≤ −65° C. | testing |

### Example 23: Formulation, Packaging, and Storage of DF hIL12-Fc Si

The DF hIL12-Fc si drug product manufacturing process flow diagram, indicating manufacturing steps and in-process controls (IPCs), is shown in FIG. 32. Filtration and filling are performed following aseptic procedures that meet applicable standards described in ICH guidelines and current Good Manufacturing Practices.

### Thawing Bulk Drug Substance

The DF hIL12-Fc si Drug Substance (DS) is thawed for ≤96 hours at 2-8° C. in the dark. Complete thawing of DS is confirmed by visual examination of the bottle(s).

### Dilution to 80% of Target Batch Volume

A buffer consisting of 20 mM citrate, 6% (w/v) sucrose, 1% (w/v) mannitol, 0.01% polysorbate 80 (w/v), pH 6.0 is prepared in a 10 L glass carboy. Solid sodium citrate dihydrate, citric acid monohydrate, sucrose, and mannitol are weighed, added to WFI, and mixed to dissolution. A polysorbate 80 stock solution is prepared in WFI and added to the buffer. The pH of the buffer is tested (acceptance criteria 6.5±0.4). The buffer is diluted with WFI to the target volume, mixed, tested to confirm the pH (6.5±0.4) and osmolality, and filtered through a 0.2 μm membrane.

The weight of drug substance is used to calculate a target batch volume. The drug substance is added to buffer in a clean, 10 L glass carboy to approximately 80% of the calculated batch volume and mixed. The 80% drug product solution is tested for pH (acceptance criteria 6.5±0.3) and protein concentration by absorbance at 280 nm using an Extinction coefficient of 1.44 L/(g*cm).

The buffer components are designed to yield a pH of 6.5. If the pH does not meet acceptance criteria at either the buffer or 80% bulk drug product steps, a titration with 1N sodium hydroxide or 1N hydrochloric acid may be performed to bring the pH within the acceptance criteria.

Dilution of DF hIL12-Fc si to 1 mg mL

The protein concentration result from the previous step is used to calculate the required amount of buffer to reach a DF HIL12-FC SI concentration of 1 mg/mL. The concentration is verified by absorbance at 280 nm (acceptance criteria 1.0±0.2 mg/mL), and samples are taken to confirm the pH (acceptance criteria 6.5±0.3) and osmolality.

The compounded bulk drug product solution is passed through a sterile 0.2 μm filter into a clean, 10 L glass carboy for bioburden reduction, and held until sterile filtration and filling. Samples for pre-filtration bioburden are removed from the 10 L glass carboy.

### Sterile Filtration

The bulk drug product is filtered through two filter capsules in series, each filter capsule consisting of a 0.45 μm polyethersulfone (PES) pre-filter membrane and a 0.2 μm PES sterilizing membrane. The drug product is filtered into a sterile, disposable fill bag inside a controlled Grade B area of the filling suite. Both sterilizing filter capsules are tested for integrity by bubble point after filtration (acceptance criteria ≥3200 mbar, using WFI).

Filling into Vials

The bulk drug product solution is filled from the disposable bag residing immediately outside of the restricted access barrier system (RABS). The product is filled into ready-to-use 2R borosilicate type I vials inside the controlled, Grade A RABS area of the filling suite.

The vials are stoppered with sterilized, 13 mm serum stoppers and capped with 13 mm aluminum overseals. Fill volume of the vials is verified by weight checks of 100% of the batch during filling operations (acceptance criteria 1.3 mL+5%). After filling, vials are moved to 2-8° C. storage.

### Visual Inspection, Packaging and Storage

The filled vials undergo 100% manual visual inspection for container, closure, and product defects, followed by an Acceptance Quality Limit inspection (AQL). The inspected vials are bulk packaged and stored at 2-8° C. prior to shipment.

### Example 24: Formulation Analysis

### Buffer Analysis

The formulations listed in Table 29 were evaluated to assess the effects of various buffer and pH conditions on the stability of DF hIL12-Fc si. DF hIL12-Fc si was buffer exchanged using centrifugal ultrafiltration devices (Amicon Ultra-4 30 k MWCO) into the buffers listed in Table 29 to a target protein concentration of 1 mg/mL. Following the final buffer-exchange, the protein concentration was measured using UV-Visible spectroscopy with the sponsor-provided extinction coefficient (1.43 mL/cm*mg). The samples were then split into three equal sized aliquots. One aliquot was stored at 2-8° C. and the other two were stored at 50° C. The aliquot stored at a 2-8° C. and one of the aliquots at 50° C. were both removed at 1 week for testing as listed in Table 30. The other vial at 50° C. was removed after 2 weeks and stored at −75° C.

TABLE 29
Buffers screened for DF hIL12-Fc si preformulation

| Buffer | pH |
|---|---|
| Succinate (20 mM) | 5.5 |
| pKa = 5.64 | 6.5 |
| Citrate (20 mM) | 5.5 |
| pKa = 6.4 | 6.0 |
| | 7.0 |
| Histidine (20 mM) | 6.5 |
| pKa = 6.04 | 7.0 |
| Phosphate (20 mM) | 6.5 |
| pKa = 7.2 | 7.0 |
| | 7.5 |
| Tris (20 mM) | 7.5 |
| pKa = 8.1 | 8.0 |

TABLE 30
Assay panel

| Test Panel | Assay Volume (μL) |
|---|---|
| Visual Appearance | In Vial |
| DSF | 100 |
| A₂₈₀ | 100 |
| DLS | 100 |
| SEC-HPLC | 100 |
| ATM-2606 | |
| CE (reduced) | 100 |
| ATM-2747 | |

### Results

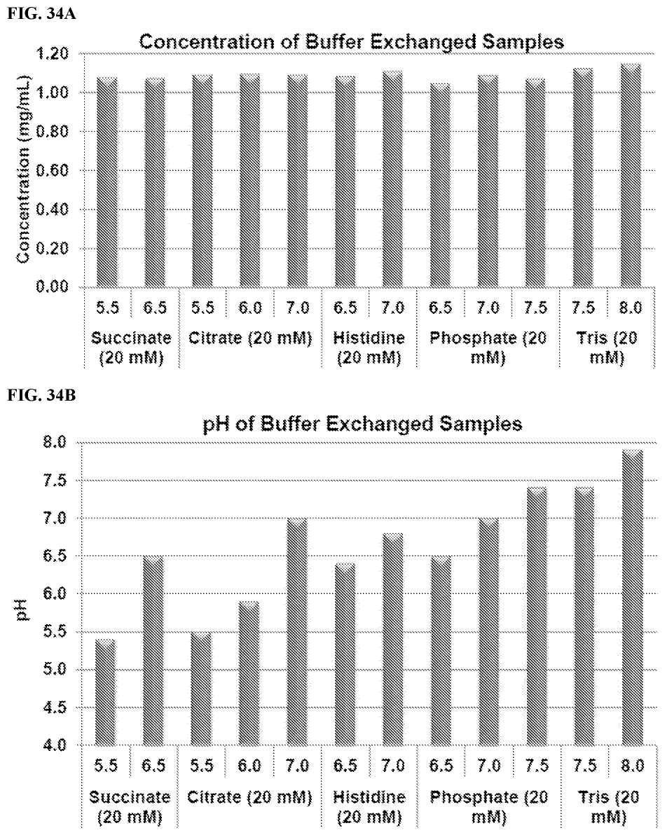

Samples of DF hIL12-Fc si were buffer exchanged into 12 buffer/pH conditions. The pH and concentration of the samples were immediately assessed. After which, the samples were aliquoted and stored at 2-8° C. and 50° C. After a one-week incubation, the samples were then assessed per the assay panel in Table 30. The results are shown below, and in FIGS. 34A-42B.

TABLE 31
Concentration of DF hIL12-Fc si (buffer exchange)

| Buffer | Target pH | Concentration (mg/mL) |
|---|---|---|
| Succinate (20 mM) | 5.5 | 1.08 |
| pKa = 5.64 | 6.5 | 1.07 |
| Citrate (20 mM) | 5.5 | 1.09 |
| pKa = 6.4 | 6.0 | 1.09 |
| | 7.0 | 1.09 |
| Histidine (20 mM) | 6.5 | 1.08 |
| pKa = 6.04 | 7.0 | 1.11 |
| Phosphate (20 mM) | 6.5 | 1.05 |
| pKa = 7.2 | 7.0 | 1.09 |
| | 7.5 | 1.07 |
| Tris (20 mM) | 7.5 | 1.12 |
| pKa = 8.1 | 8.0 | 1.15 |

Please note:
concentration = ((A₂₈₀-A₃₂₀)/1.43)*1.0 (cm path length) *
Dilution Factor (if applicable)

TABLE 32
pH values (buffer exchange)

| Buffer | Target pH | Buffer pH | Sample pH |
|---|---|---|---|
| Succinate (20 mM) | 5.5 | 5.4 | 5.4 |
| pKa = 5.64 | 6.5 | 6.4 | 6.5 |
| Citrate (20 mM) | 5.5 | 5.5 | 5.5 |
| pKa = 6.4 | 6.0 | 5.9 | 5.9 |
| | 7.0 | 6.9 | 7.0 |
| Histidine (20 mM) | 6.5 | 6.4 | 6.4 |
| pKa = 6.04 | 7.0 | 6.9 | 6.8 |
| Phosphate (20 mM) | 6.5 | 6.5 | 6.5 |
| pKa = 7.2 | 7.0 | 6.9 | 7.0 |
| | 7.5 | 7.5 | 7.4 |
| Tris (20 mM) | 7.5 | 7.4 | 7.4 |
| pKa = 8.1 | 8.0 | 7.9 | 7.9 |

### Visual Appearance (1-Week Incubation)

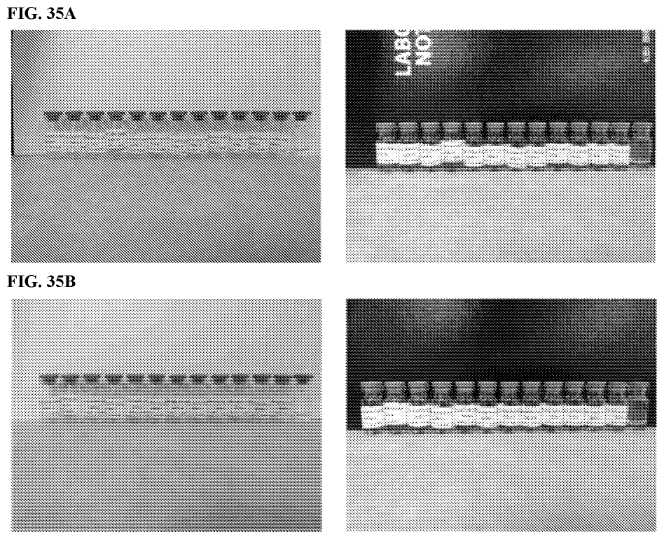

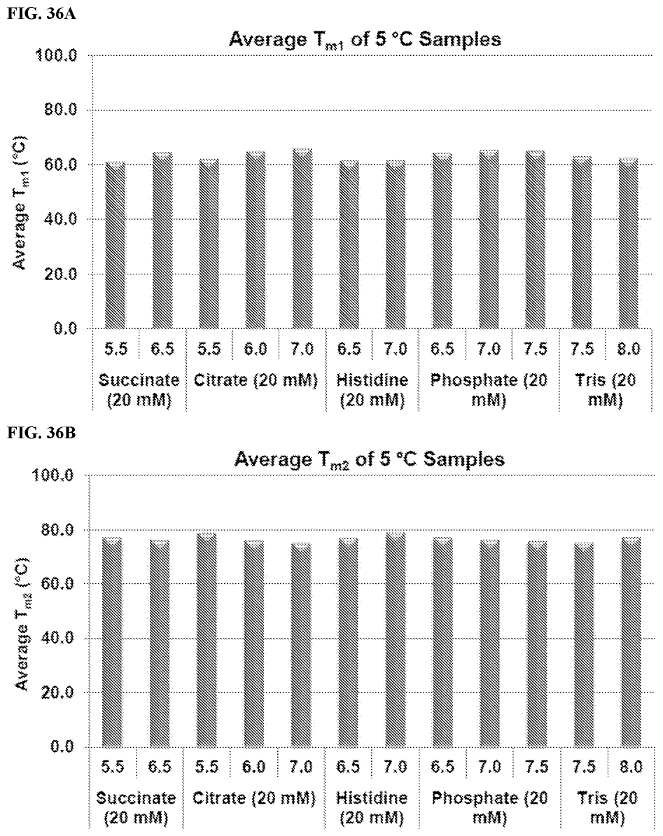

Visual appearances of all samples were assessed 1 week after incubating the samples at 5° C. or at 50° C. All samples were colorless, clear liquids, free of visible particulates (see FIGS. 35A-35B).

### Differential Scanning Fluorimetry (DSF) (1-Week Incubation)

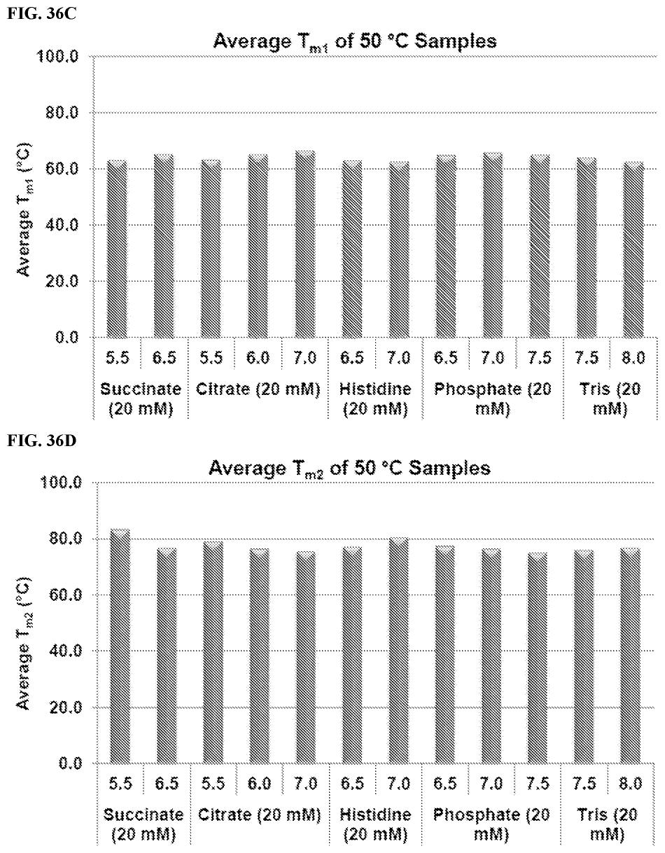

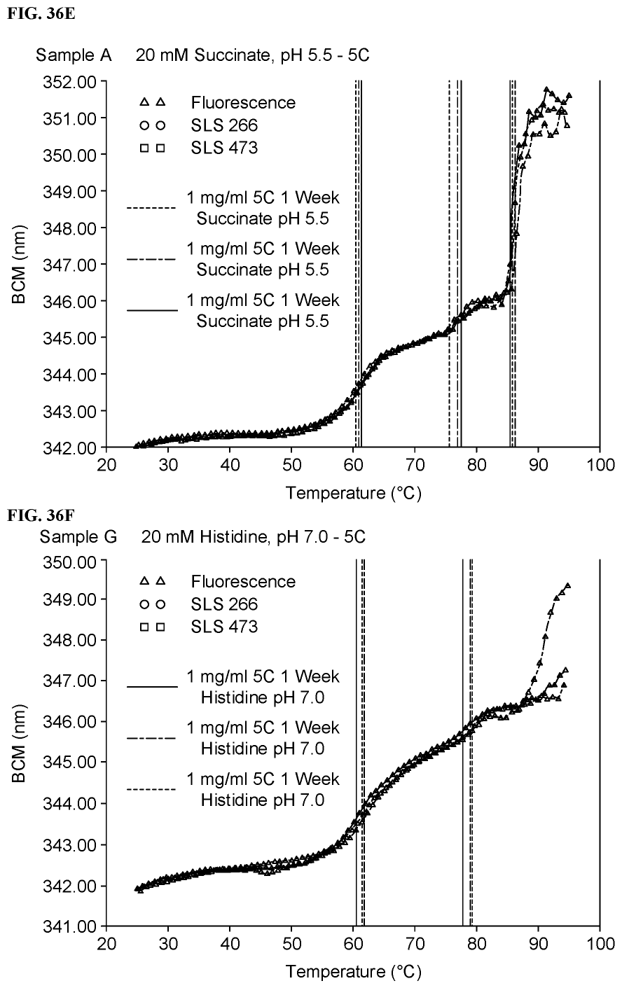

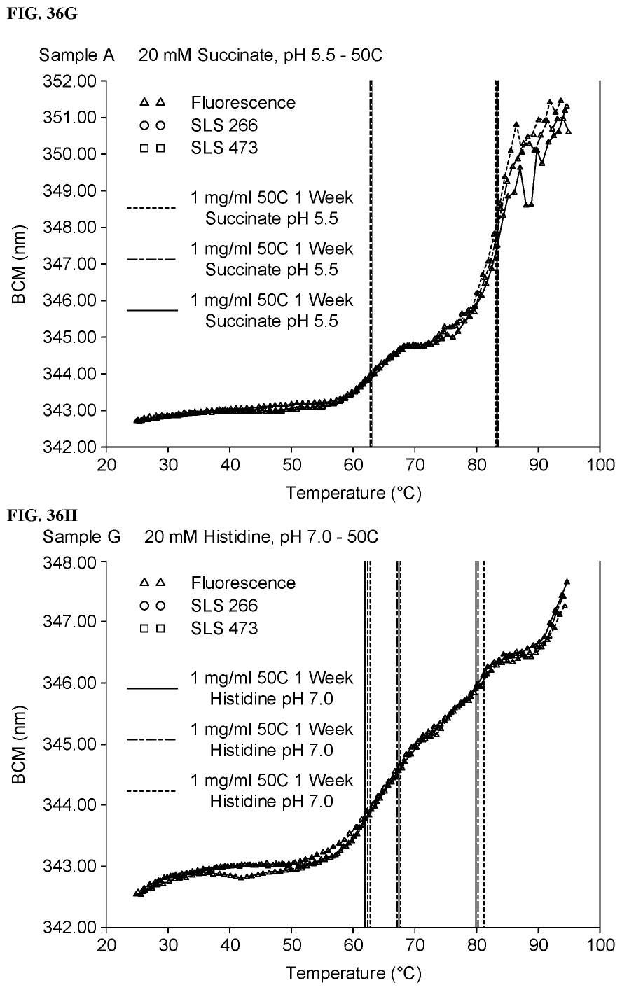

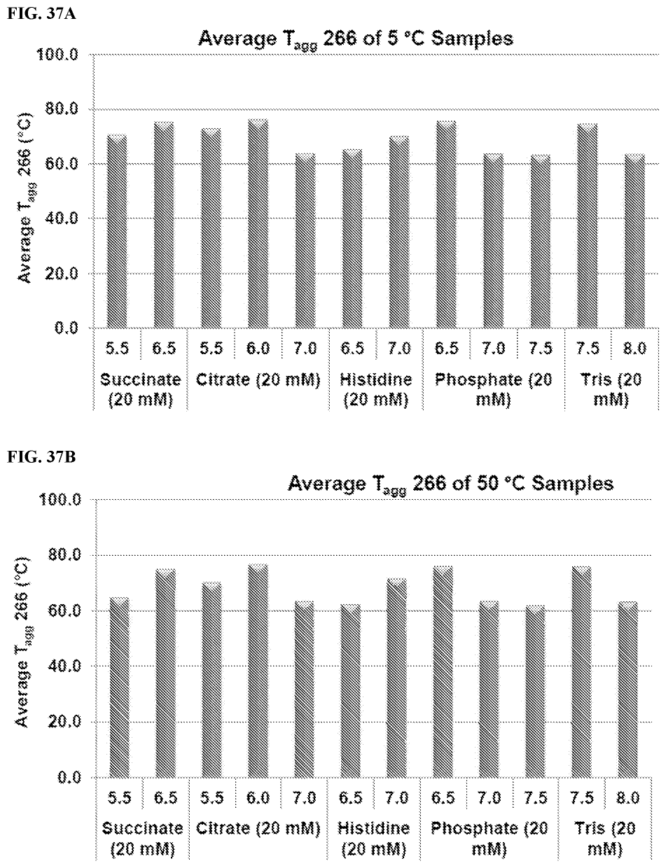

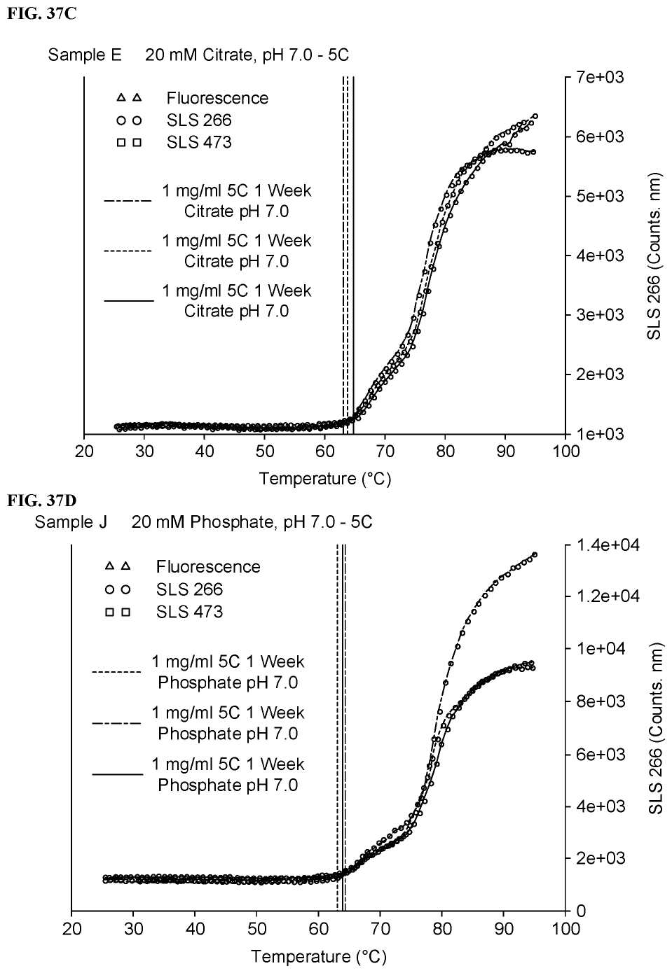

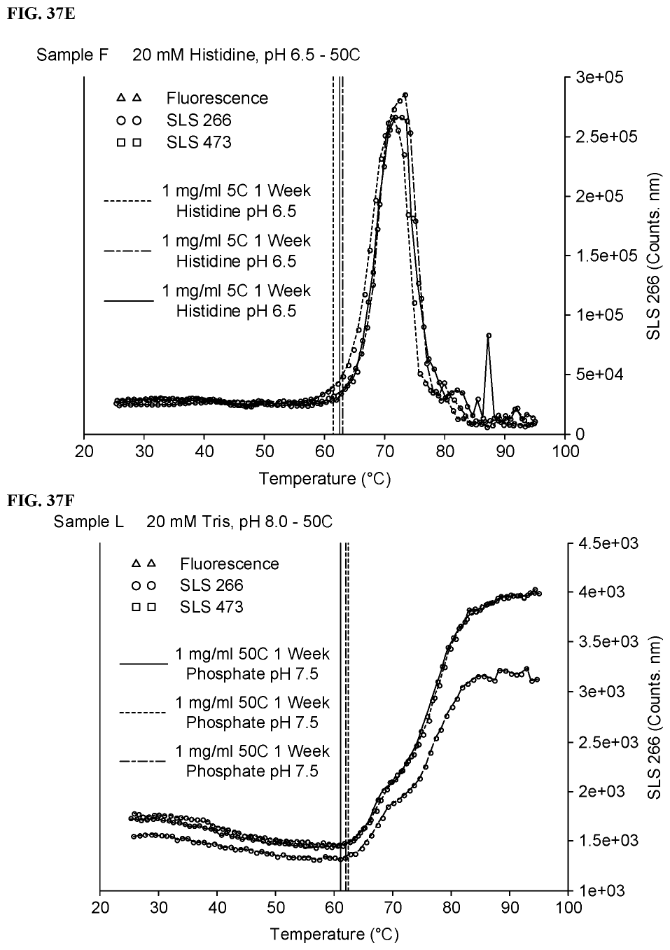

The DSF experiments were preformed using an Unchained Labs UNcle. Samples were evaluated for their thermal stability. DF hIL12-Fc si thermal unfolding (Tₘ) and onset of aggregation (T_{agg}) were monitored by evaluating changes in intrinsic protein fluorescence and static light scattering (SLS at 266 nm), respectively, as a function of temperature. Samples were evaluated in triplicate, and the triplicate results were averaged. Samples were analyzed over a temperature ramp of 25° C.-95° C. at a constant linear ramp rate of 0.5° C./min. See Tables 33-36, below, and FIGS. 36A-37D.

TABLE 33
Determined DSF Tₘ Temperatures
(1-week incubation at 5° C.)

| | | Average | Average | Average |
|---|---|---|---|---|
| Buffer | pH | Tₘ₁ (° C.) | Tₘ₂ (° C.) | Tₘ₃ (° C.) |
| Succinate (20 mM) | 5.5 | 61.0 | 76.7 | 85.8 |
| pKa = 5.64 | 6.5 | 64.4 | 75.9 | N/A |
| Citrate (20 mM) | 5.5 | 62.0 | 78.6 | N/A |
| pKa = 6.4 | 6.0 | 64.6 | 75.7 | N/A |
| | 7.0 | 65.8 | 74.7 | N/A |
| Histidine (20 mM) | 6.5 | 61.3 | 76.5 | N/A |
| pKa = 6.04 | 7.0 | 61.3 | 78.8 | N/A |
| Phosphate (20 mM) | 6.5 | 64.0¹ | 76.8¹ | N/A¹ |
| pKa = 7.2 | 7.0 | 65.1 | 76.0 | N/A |
| | 7.5 | 64.8 | 75.4 | N/A |
| Tris (20 mM) | 7.5 | 62.9 | 75.0 | 80.6 |
| pKa = 8.1 | 8.0 | 62.2 | 76.8 | N/A |

¹One of the triplicate wells was excluded from analysis. A low intensity reading occurred around Tₘ₁, negatively affecting the differential and preventing proper analysis of Tₘ₁.

TABLE 34
Determined DSF Tₘ Temperatures (1-week incubation at 50° C.)

| | | Average | Average | Average |
|---|---|---|---|---|
| Buffer | pH | Tₘ₁ (° C.) | Tₘ₂ (° C.) | Tₘ₃ (° C.) |
| Succinate (20 mM) | 5.5 | 63.0 | 83.3 | N/A |
| pKa = 5.64 | 6.5 | 65.1 | 76.7 | N/A |
| Citrate (20 mM) | 5.5 | 63.0 | 79.1 | N/A |
| pKa =6.4 | 6.0 | 65.0¹ | 76.5¹ | N/A¹ |
| | 7.0 | 66.4 | 75.5 | N/A |
| Histidine (20 mM) | 6.5 | 62.8 | 77.3 | N/A |
| pKa = 6.04 | 7.0 | 62.4 | 80.5² | N/A¹ |
| Phosphate (20 mM) | 6.5 | 64.8 | 77.5 | N/A |
| pKa = 7.2 | 7.0 | 65.5 | 76.4 | N/A |
| | 7.5 | 64.9 | 75.1 | N/A |
| Tris (20 mM) | 7.5 | 63.9 | 76.0 | 83.4 |
| pKa = 8.1 | 8.0 | 62.4 | 76.8 | N/A |

¹One of the triplicate wells was excluded from analysis. Data acquired for this well was indicative of either an air bubble or misread, preventing proper analysis.
²An additional Tₘ (67.4° C.) was identified between Tₘ₁ and Tₘ₂. The Tm was not reported as Tₘ₂ given its low temperature as well as poor differential and separation from Tₘ₁.

TABLE 35
Determined DSF T_{agg} 266 Temperatures
(1-week incubation at 5° C.)

| | | T_{agg} 266(° C.) |
|---|---|---|
| Buffer | pH | Average |
| Succinate (20 mM) | 5.5 | 70.7 |
| pKa = 5.64 | 6.5 | 75.3 |
| Citrate (20 mM) | 5.5 | 72.9 |
| pKa = 6.4 | 6.0 | 76.1 |
| | 7.0 | 63.9 |
| Histidine (20 mM) | 6.5 | 65.4 |
| pKa = 6.04 | 7.0 | 70.1 |
| Phosphate (20 mM) | 6.5 | 75.8 |
| pKa = 7.2 | 7.0 | 63.7 |
| | 7.5 | 63.2 |
| Tris (20 mM) | 7.5 | 74.8 |
| pKa = 8.1 | 8.0 | 63.4 |

TABLE 36
Determined DSF T_{agg} 266 Temperatures
(1-week incubation at 50° C.)

| Buffer | pH | T_{agg} 266(° C.) Average |
|---|---|---|
| Succinate (20 mM) | 5.5 | 64.7 |
| pKa = 5.64 | 6.5 | 74.9 |
| Citrate (20 mM) | 5.5 | 70.1 |
| pKa = 6.4 | 6.0 | 76.7 |
| | 7.0 | 63.3 |
| Histidine (20 mM) | 6.5 | 62.2 |
| pKa = 6.04 | 7.0 | 71.5 |
| Phosphate (20 mM) | 6.5 | 76.0 |
| pKa = 7.2 | 7.0 | 63.4 |
| | 7.5 | 61.8 |
| Tris (20 mM) | 7.5 | 75.8 |
| pKa = 8.1 | 8.0 | 63.1 |

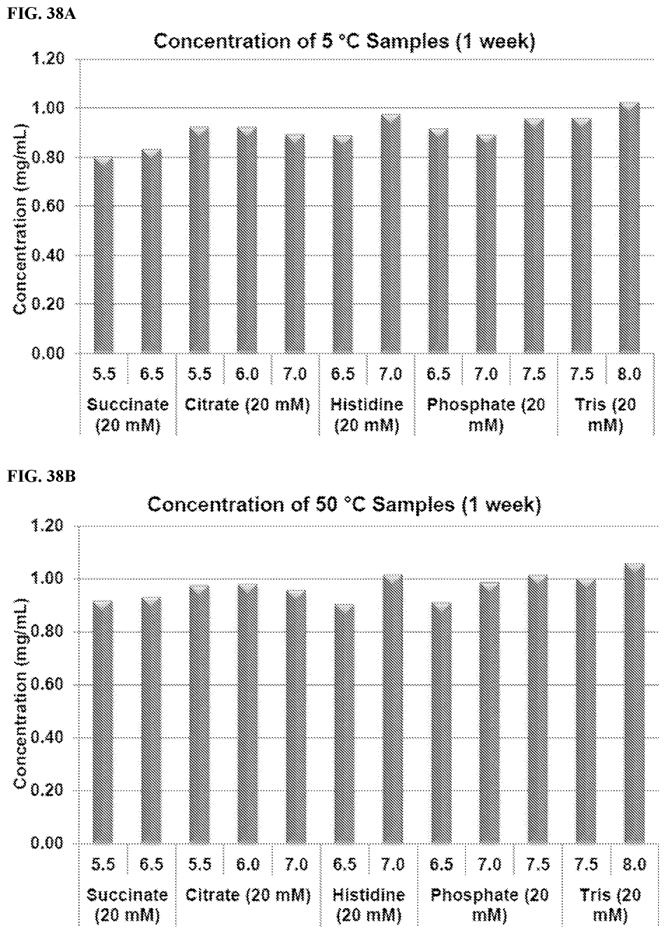

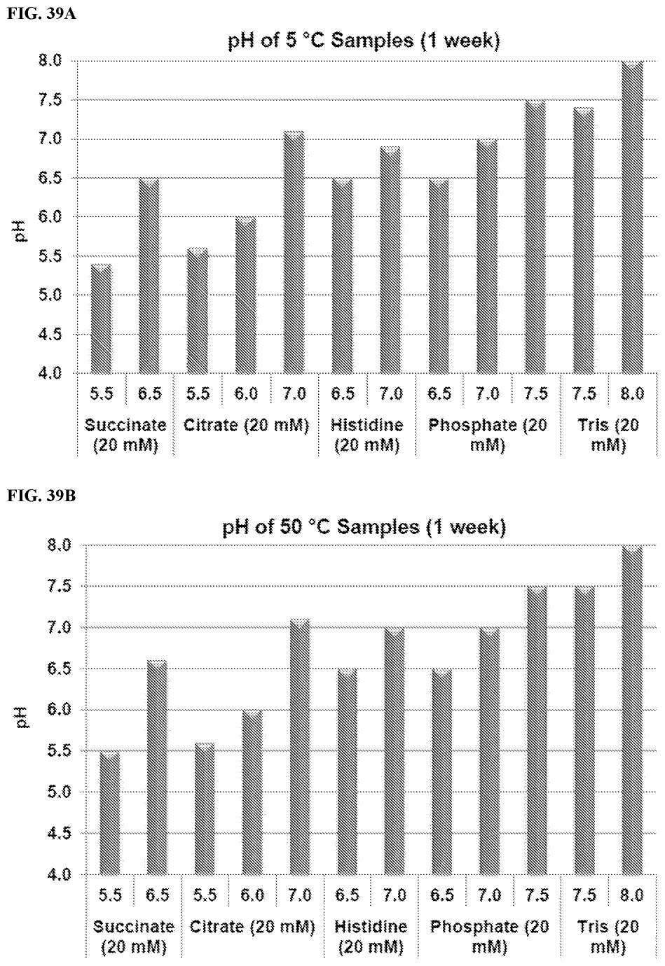

Concentrations of DF hIL12-Fc si and pH were assessed in the various buffer formulations after 1 week of incubation at 5° C. and 50° C. See Tables 37-40 and FIGS. 38A-39B.

TABLE 37
Concentration of DF hIL12-Fc si (1-week incubation at 5° C.)

| Buffer | Target pH | Concentration (mg/mL) |
|---|---|---|
| Succinate (20 mM) | 5.5 | 0.80 |
| pKa = 5.64 | 6.5 | 0.83 |
| Citrate (20 mM) | 5.5 | 0.92 |
| pKa =6.4 | 6.0 | 0.92 |
| | 7.0 | 0.89 |
| Histidine (20 mM) | 6.5 | 0.89 |
| pKa = 6.04 | 7.0 | 0.97 |
| Phosphate (20 mM) | 6.5 | 0.91 |
| | 7.0 | 0.89 |
| pKa = 7.2 | 7.5 | 0.96 |
| Tris (20 mM) | 7.5 | 0.96 |
| pKa = 8.1 | 8.0 | 1.02 |

Please note:
concentration = ((A₂₈₀-A₃₂₀)/1.43)*1.0 (cm path length) * Dilution Factor (if applicable)

TABLE 38
Concentration of DF hIL12-Fc si (1-week incubation at 50° C.)

| Buffer | Target pH | Concentration (mg/mL) |
|---|---|---|
| Succinate (20 mM) | 5.5 | 0.91 |
| pKa = 5.64 | 6.5 | 0.93 |
| Citrate (20 mM) | 5.5 | 0.97 |
| pKa =6.4 | 6.0 | 0.98 |
| | 7.0 | 0.96 |
| Histidine (20 mM) | 6.5 | 0.90 |
| pKa = 6.04 | 7.0 | 1.01 |
| Phosphate (20 mM) | 6.5 | 0.91 |
| pKa =7.2 | 7.0 | 0.98 |
| | 7.5 | 1.01 |
| Tris (20 mM) | 7.5 | 1.00 |
| pKa = 8.1 | 8.0 | 1.06 |

Please note:
concentration = ((A₂₈₀-A₃₂₀)/1.43)*1.0 (cm path length) * Dilution Factor (if applicable)

TABLE 39
pH values (1-week incubation at 5° C.)

| Buffer | Target pH | Buffer pH | Sample pH |
|---|---|---|---|
| Succinate (20 mM) | 5.5 | 5.4 | 5.4 |
| pKa = 5.64 | 6.5 | 6.4 | 6.5 |
| Citrate (20 mM) | 5.5 | 5.5 | 5.6 |
| pKa = 6.4 | 6.0 | 5.9 | 6.0 |
| | 7.0 | 6.9 | 7.1 |
| Histidine (20 mM) | 6.5 | 6.4 | 6.5 |
| pKa = 6.04 | 7.0 | 6.9 | 6.9 |
| Phosphate (20 mM) | 6.5 | 6.5 | 6.5 |
| pKa = 7.2 | 7.0 | 6.9 | 7.0 |
| | 7.5 | 7.5 | 7.5 |
| Tris (20 mM) | 7.5 | 7.4 | 7.4 |
| pKa = 8.1 | 8.0 | 7.9 | 8.0 |

TABLE 40
pH values (1-week incubation at 50° C.)

| Buffer | Target pH | Buffer pH | Sample pH |
|---|---|---|---|
| Succinate (20 mM) | 5.5 | 5.4 | 5.5 |
| pKa = 5.64 | 6.5 | 6.4 | 6.6 |
| Citrate (20 mM) | 5.5 | 5.5 | 5.6 |
| pKa = 6.4 | 6.0 | 5.9 | 6.0 |
| | 7.0 | 6.9 | 7.1 |
| Histidine (20 mM) | 6.5 | 6.4 | 6.5 |
| pKa = 6.04 | 7.0 | 6.9 | 7.0 |
| Phosphate (20 mM) | 6.5 | 6.5 | 6.5 |
| pKa = 7.2 | 7.0 | 6.9 | 7.0 |
| | 7.5 | 7.5 | 7.5 |
| Tris (20 mM) | 7.5 | 7.4 | 7.5 |
| pKa = 8.1 | 8.0 | 7.9 | 8.0 |

### Dynamic Light Scattering (DLS) (1-Week Incubation)

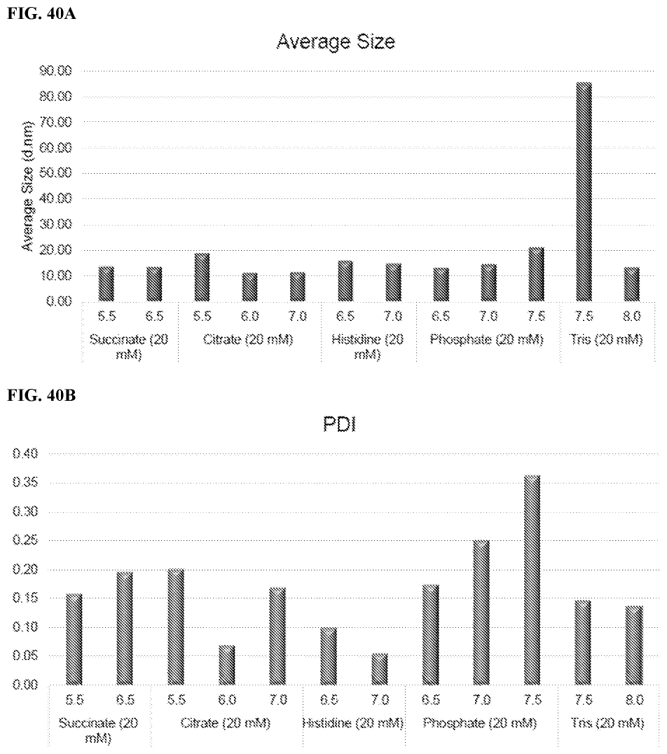

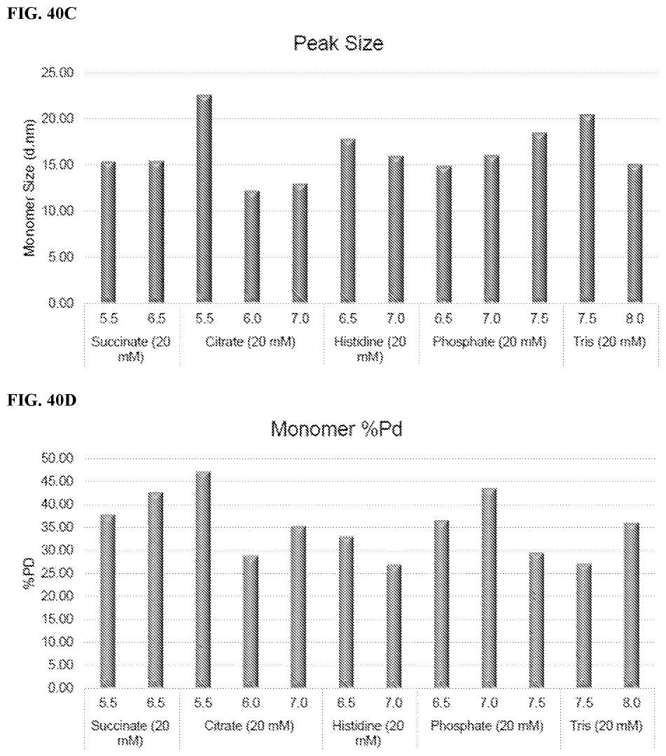

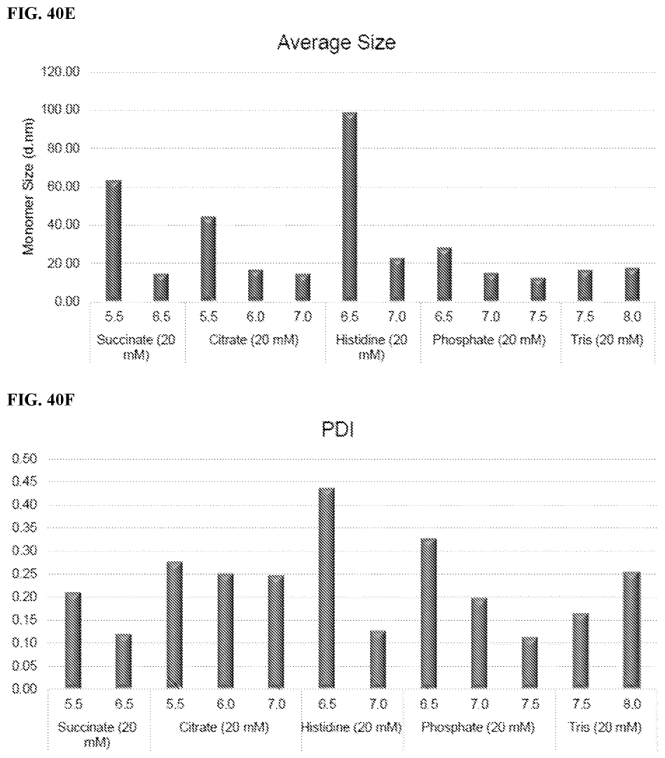

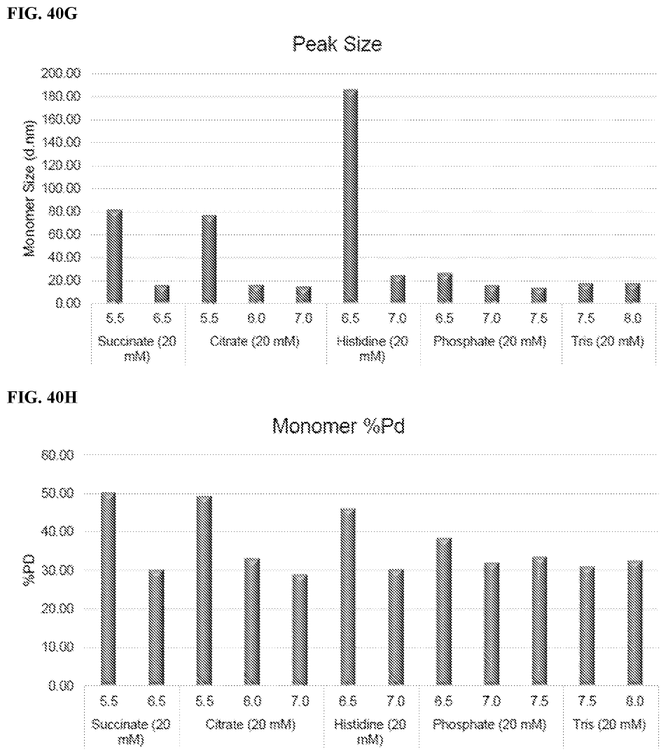

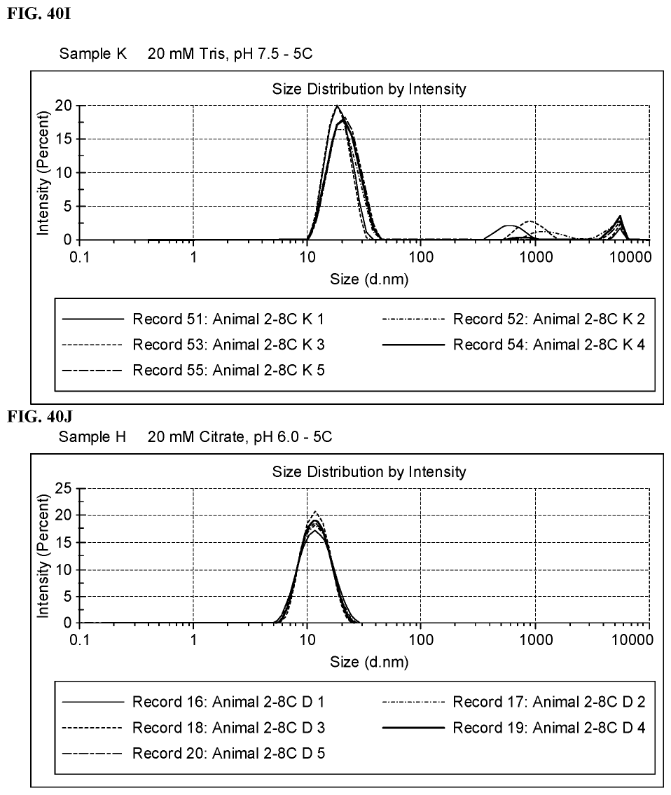

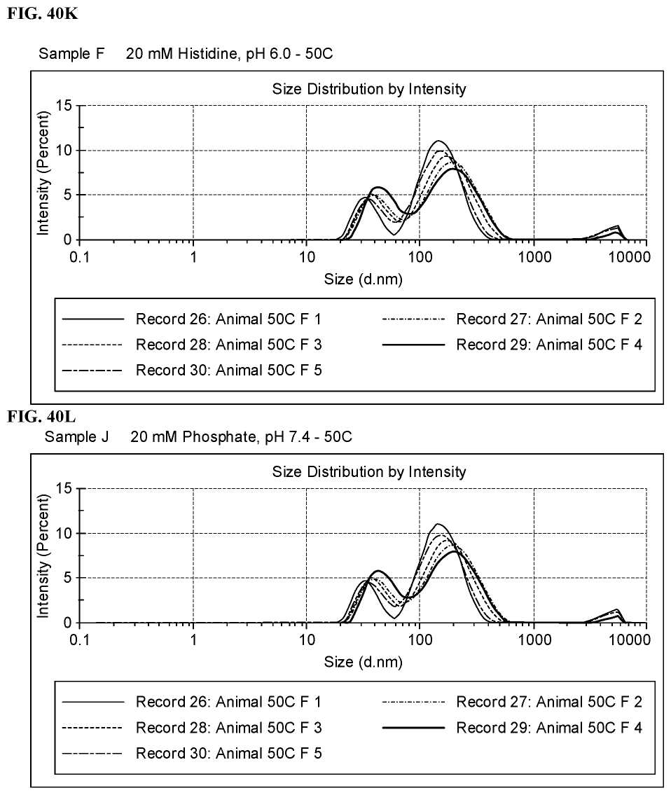

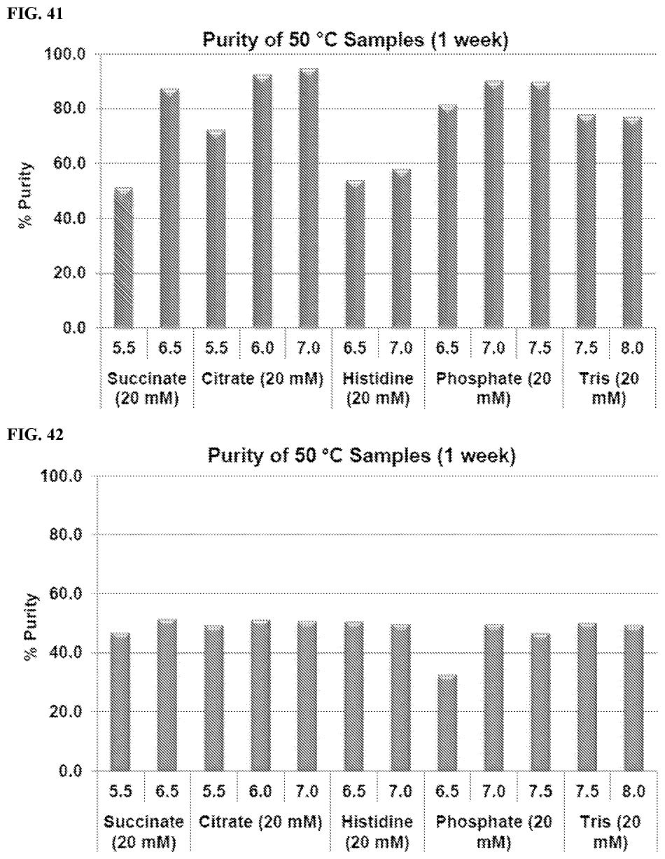

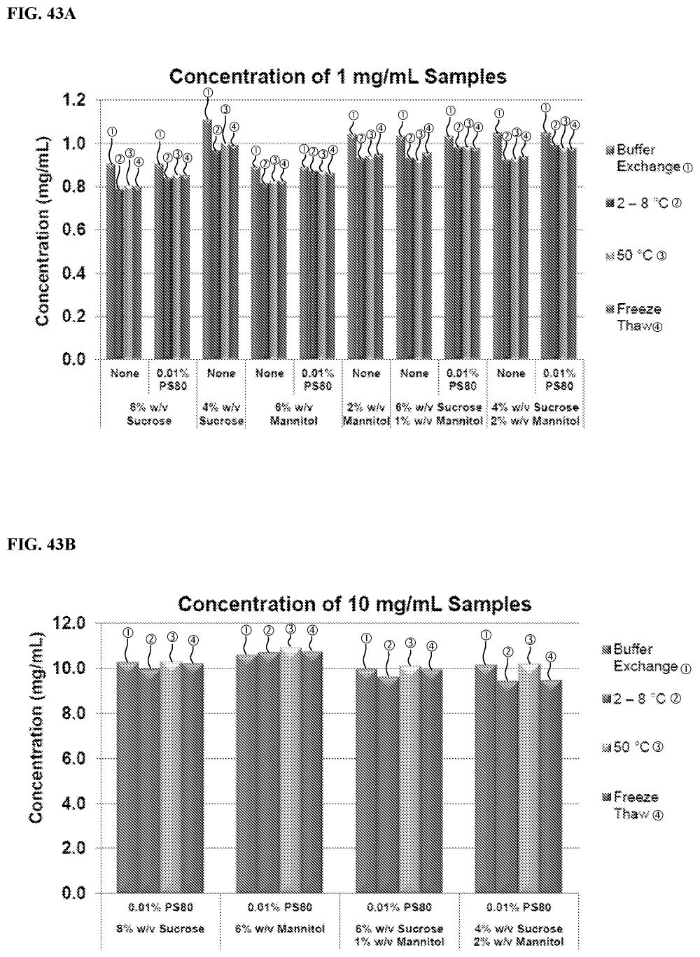

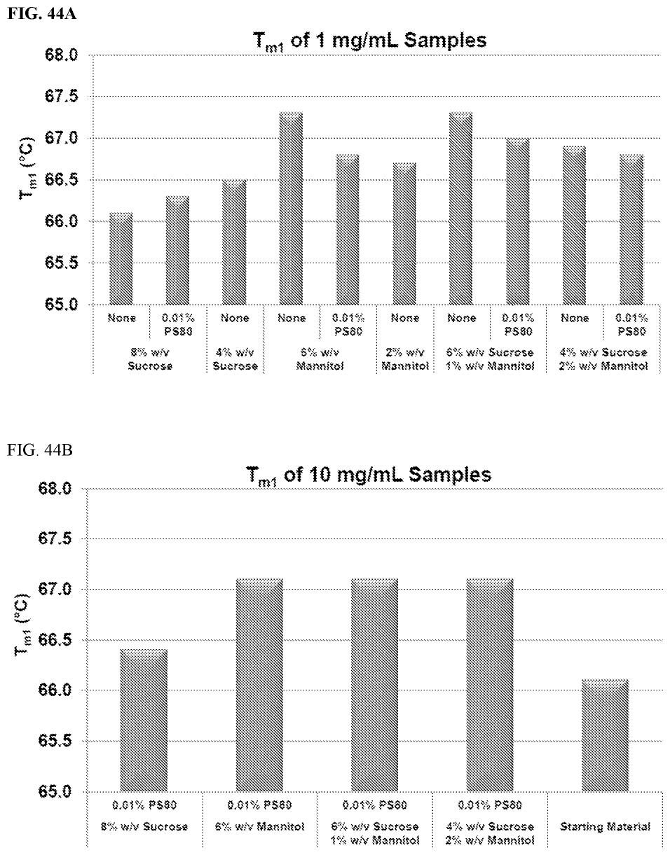

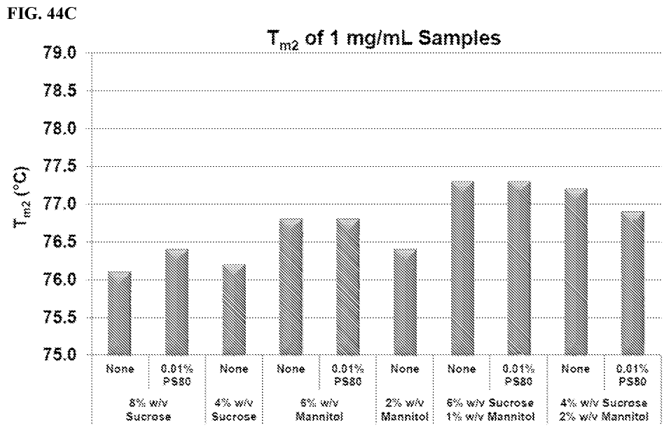

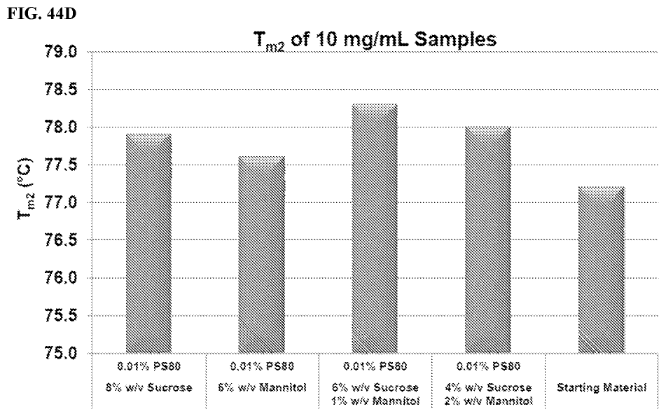

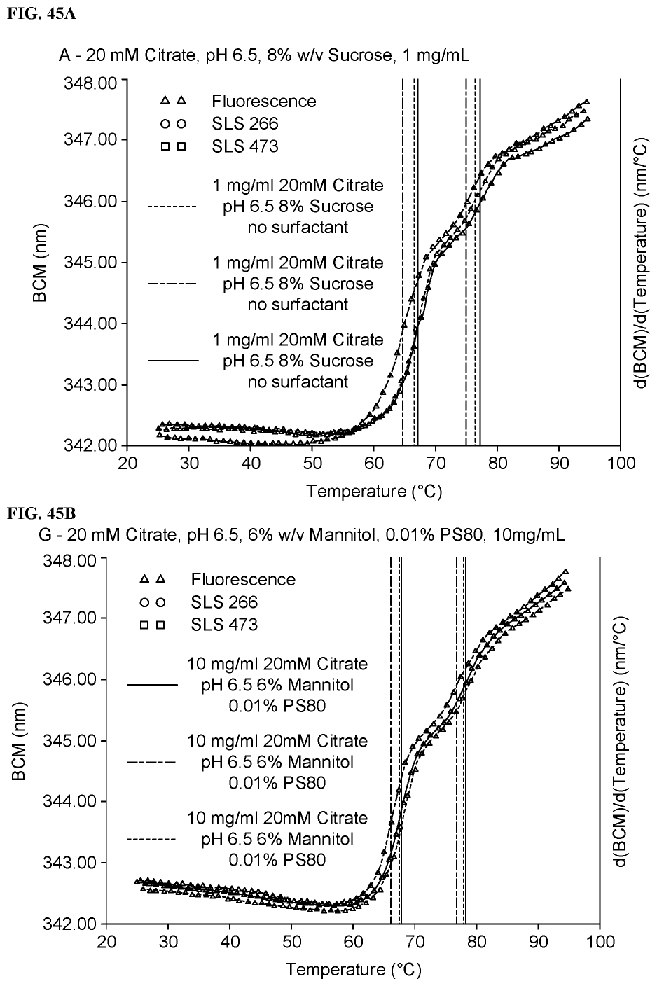

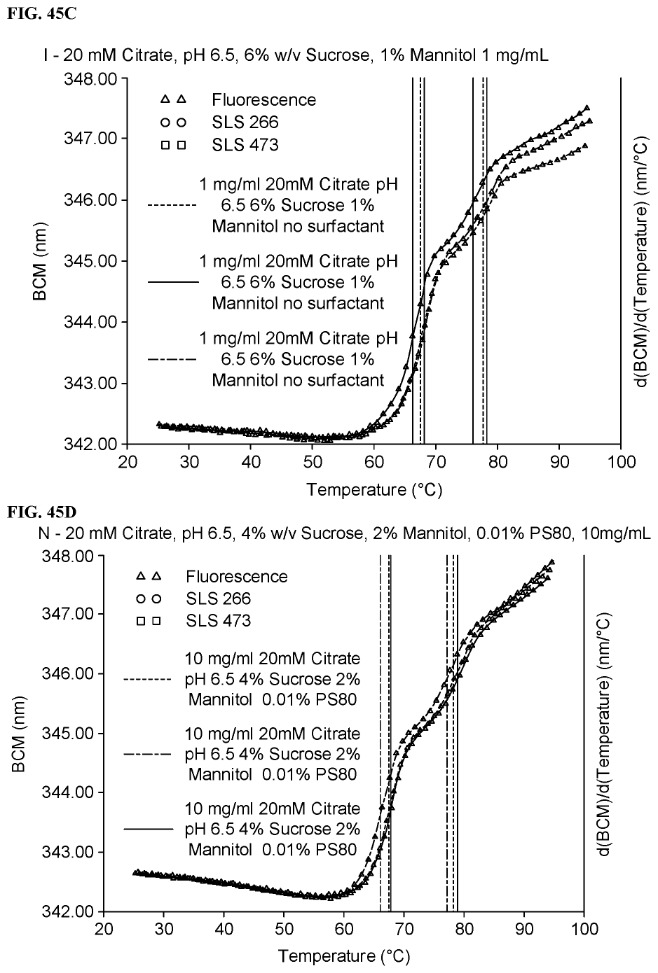

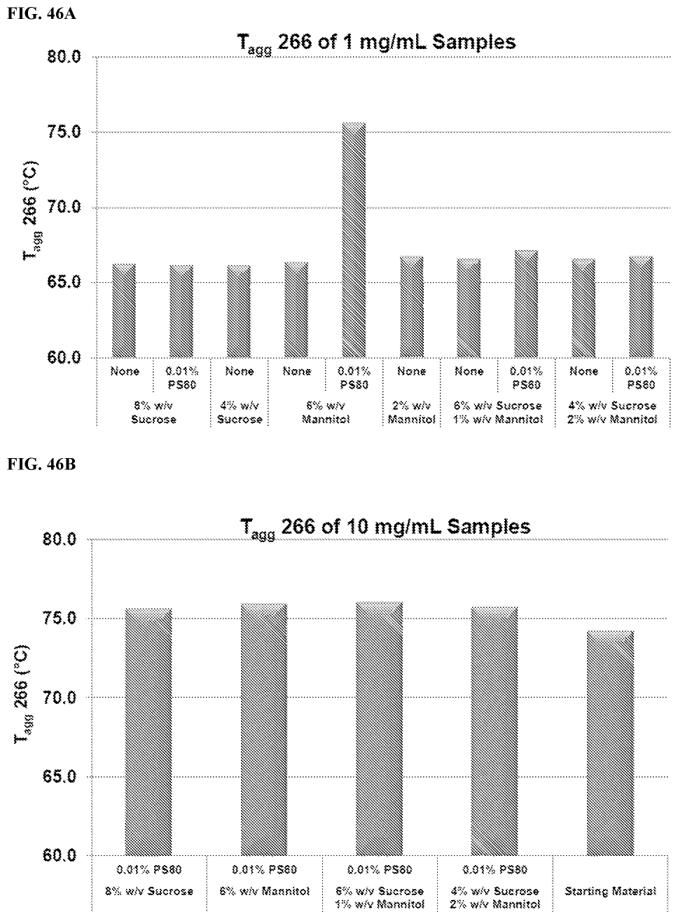

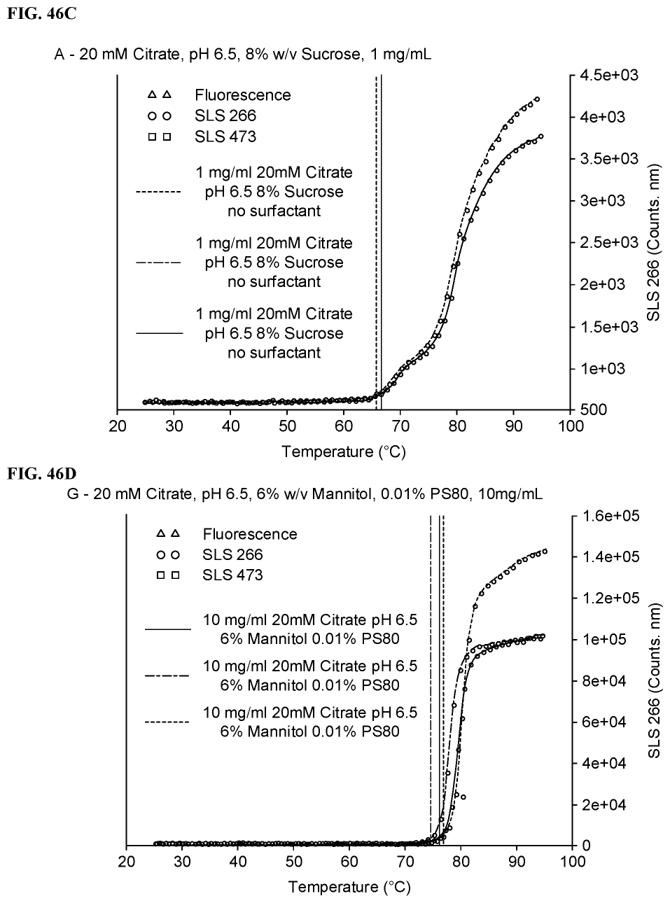

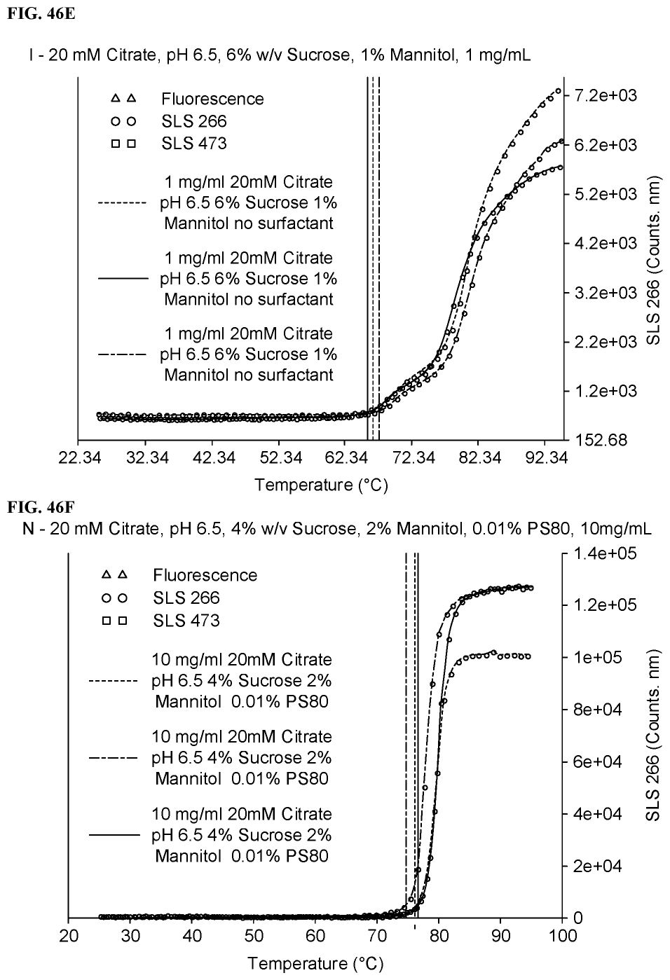

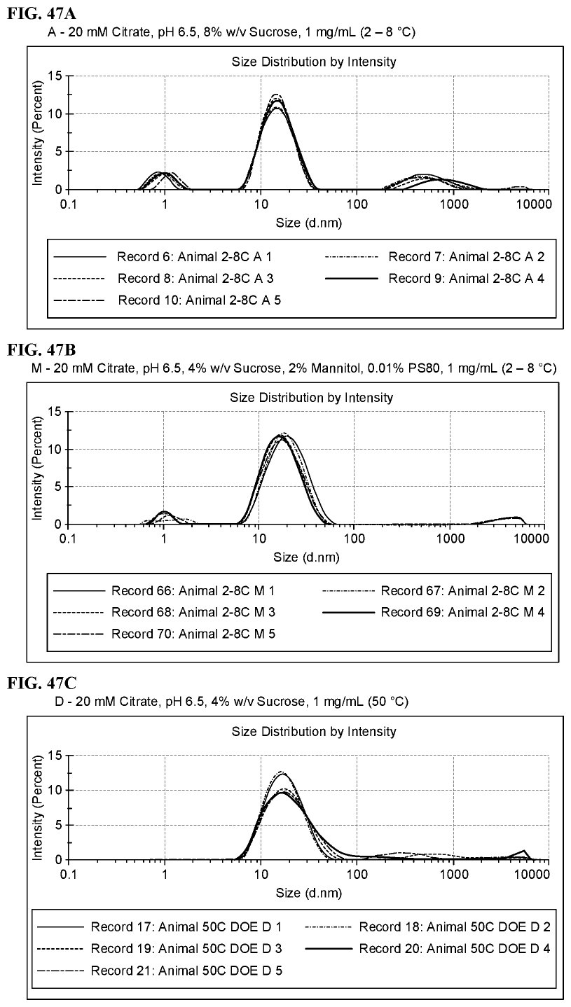

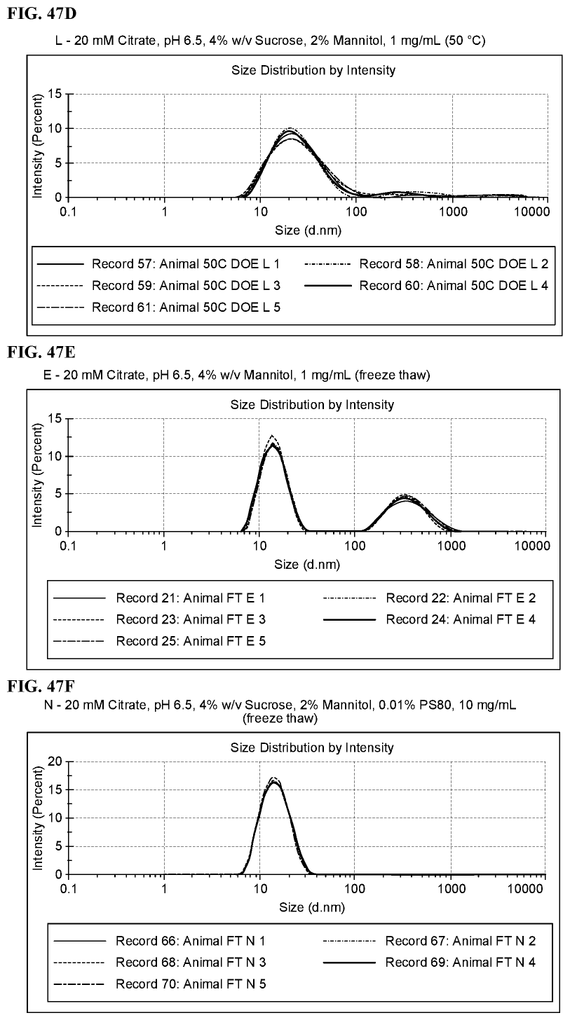

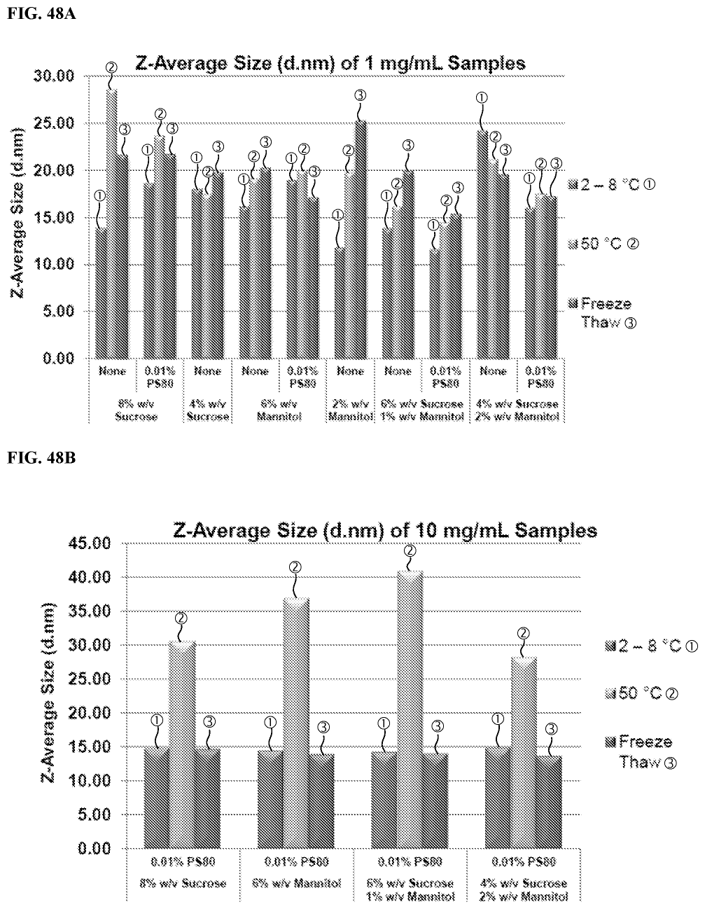

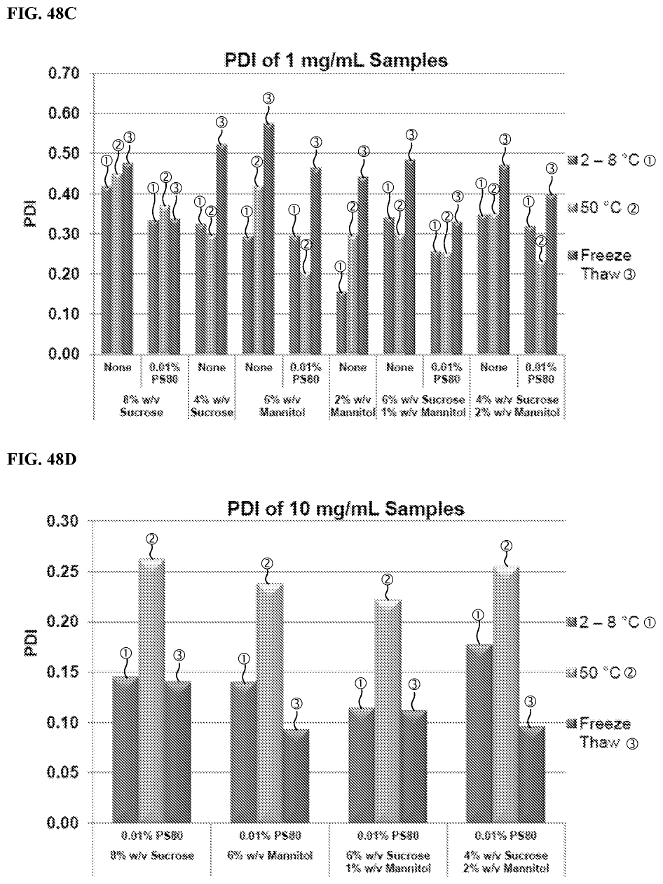

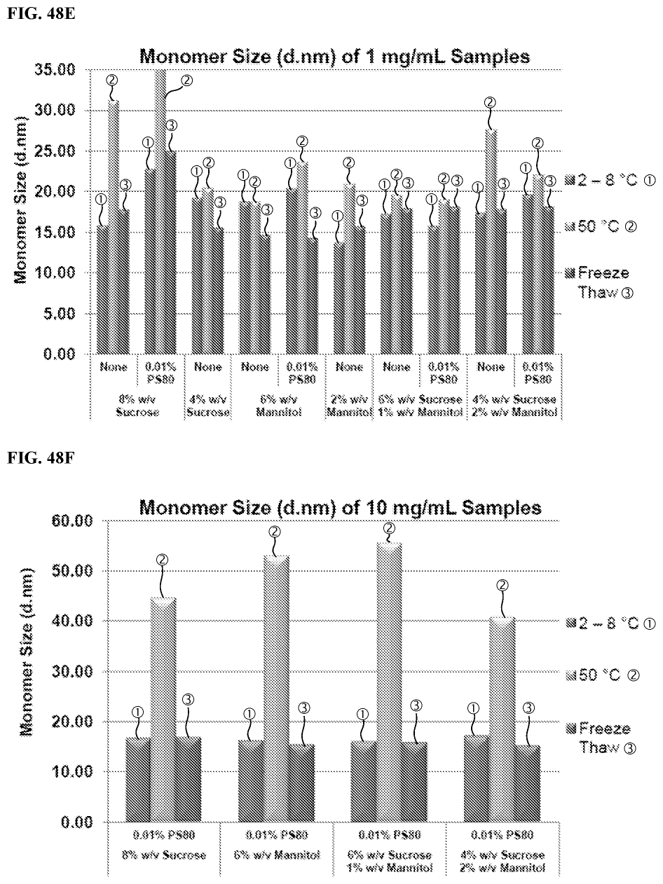

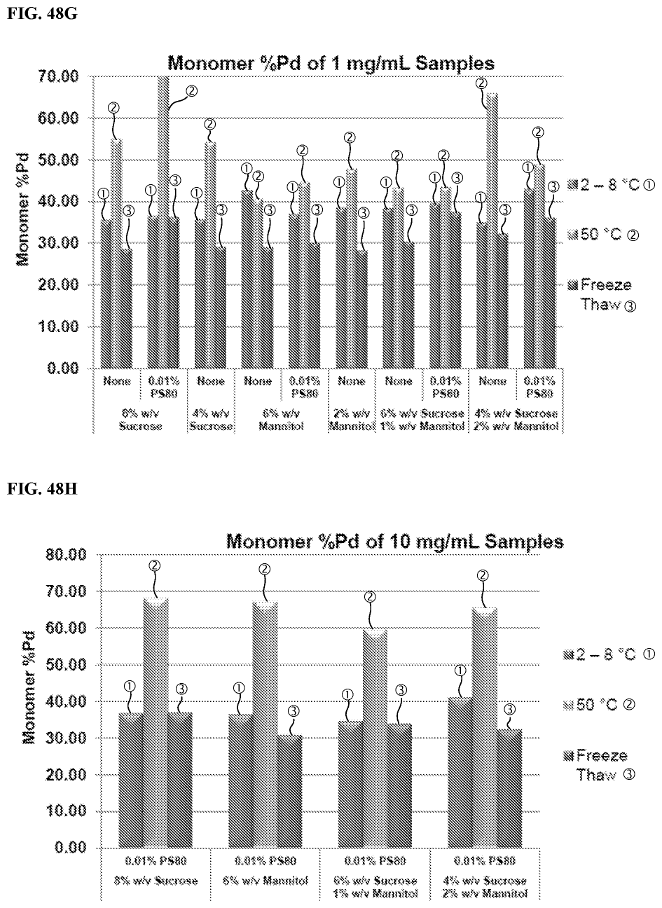

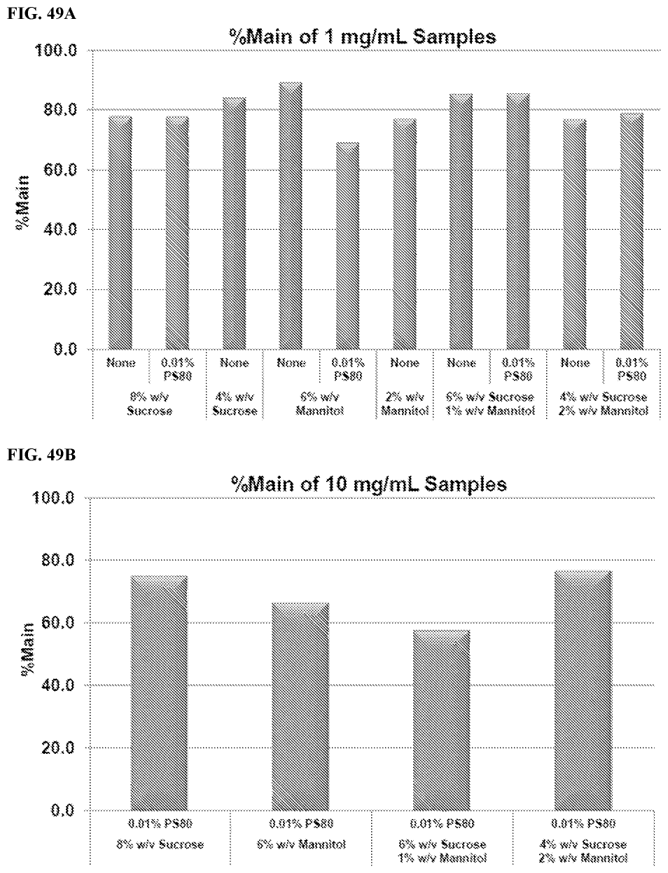

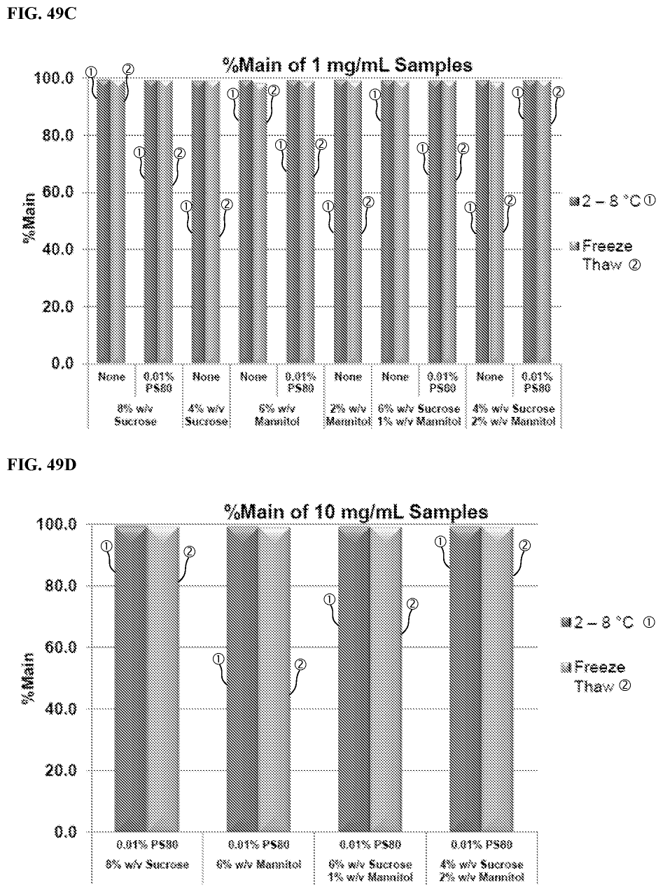

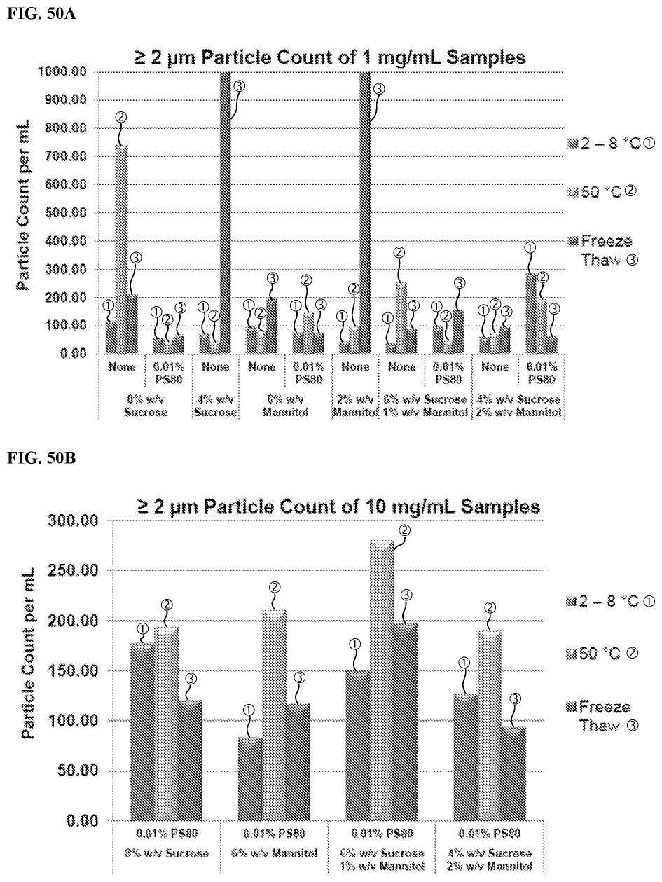

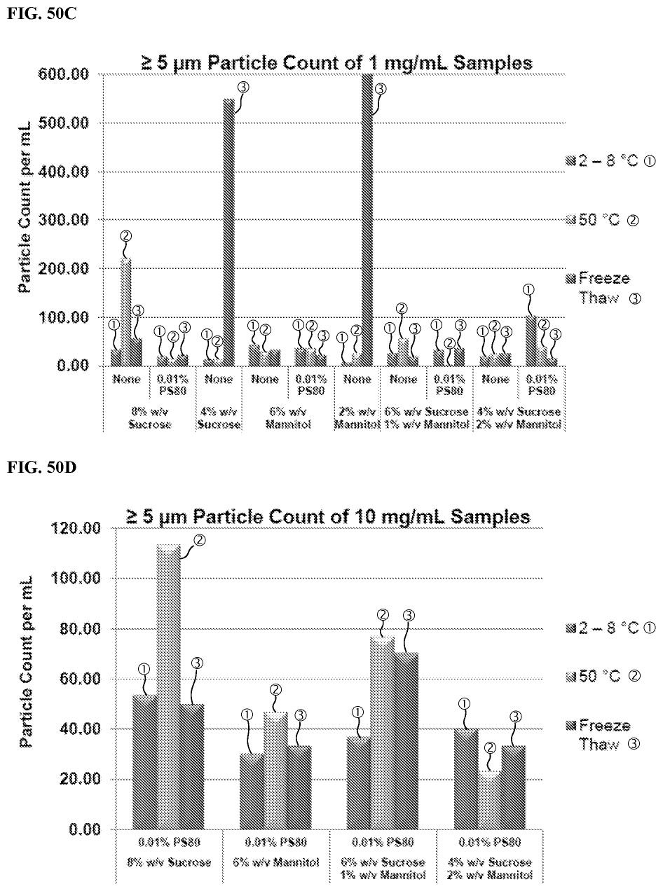

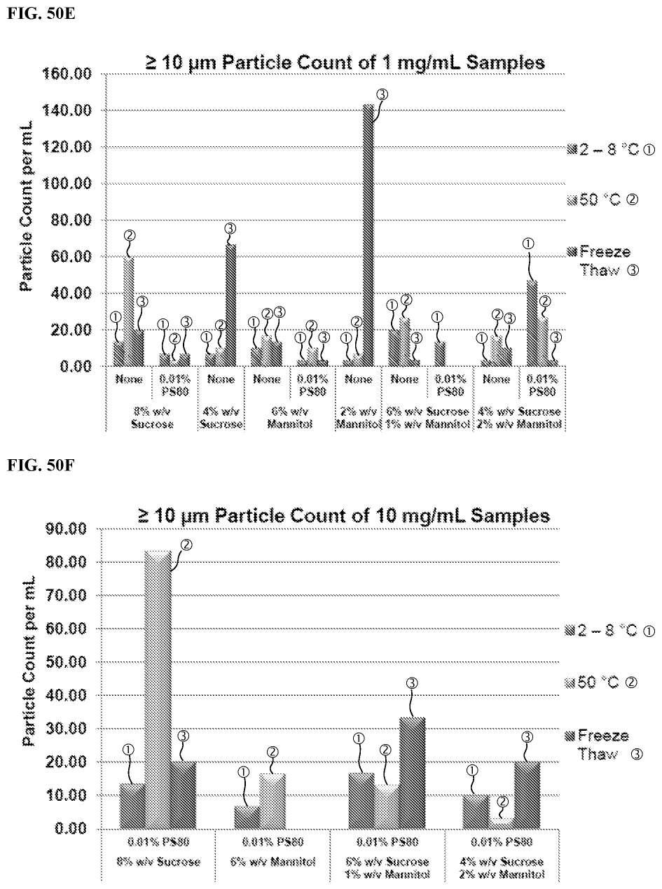

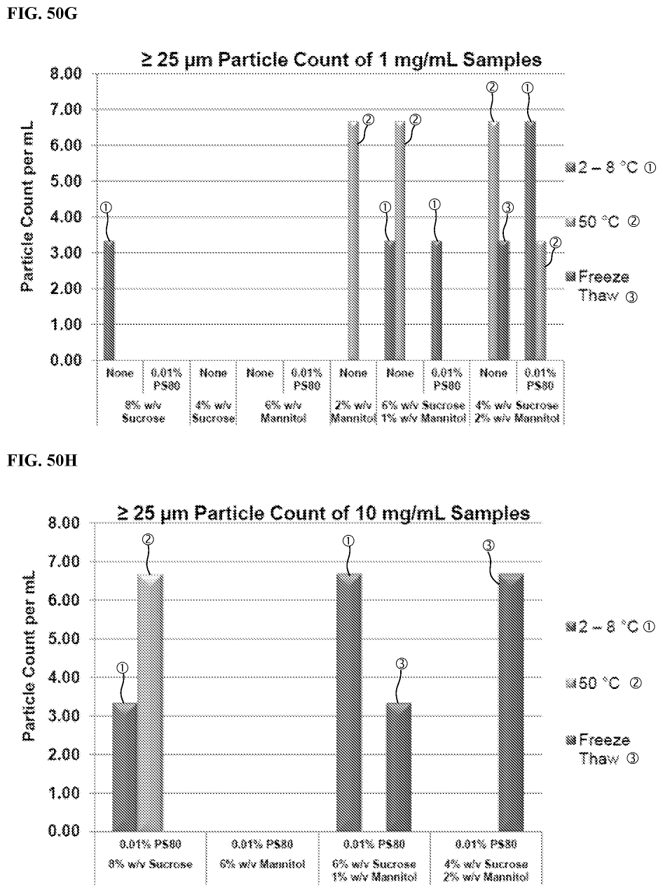

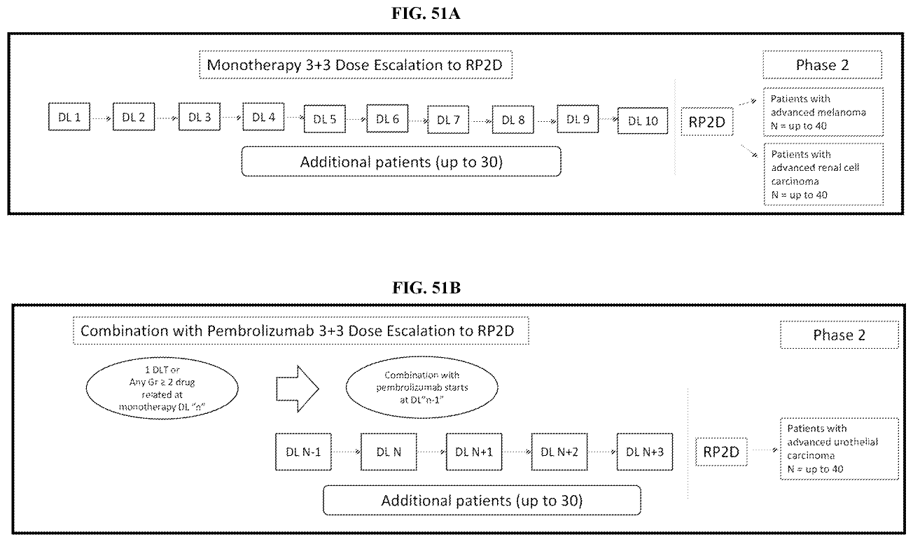

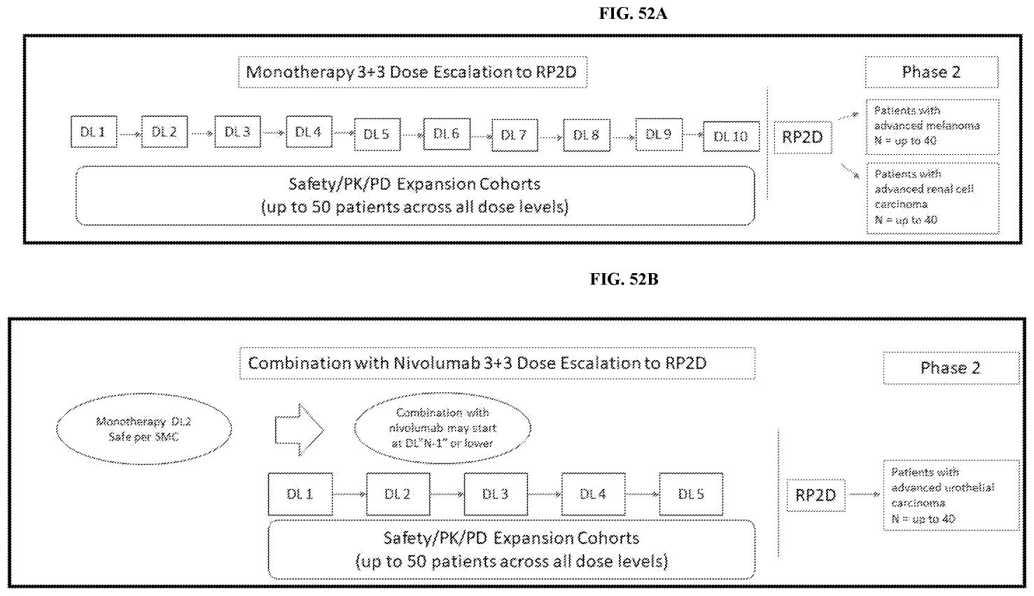

The DLS experiments were performed using a Malvern Zeta sizer. For each sample measurement, five consecutive scans were acquired at 25° C. The Z-average hydrodynamic diameter and polydispersity index (PDI) were determined from the cumulants analysis and the Stokes Einstein equation. Polydispersity is a measurement of non-uniformity in a sample. If particles are not uniform in size, a higher polydispersity will be measured. A low polydispersity index (PDI) (≤0.200) indicates greater uniformity in the size of the particle. See Tables 41-42 and FIGS. 40A-40L.

TABLE 41
Determined DLS Sizes (1-week incubation at 5° C.)

| | | Average | | Monomer | |
|---|---|---|---|---|---|
| | | Size | | Size | Monomer % |
| Buffer | pH | (d · nm) | PdI | (d · nm) | Pd |
| Succinate (20 mM) | 5.5 | 13.71 | 0.16 | 15.33 | 37.70 |
| pKa = 5.64 | 6.5 | 13.55 | 0.20 | 15.35 | 42.60 |
| Citrate (20 mM) | 5.5 | 18.94 | 0.20 | 22.52 | 47.10 |
| pKa = 6.4 | 6.0 | 11.22 | 0.07 | 12.17 | 28.80 |
| | 7.0 | 11.62 | 0.17 | 12.93 | 35.20 |
| Histidine (20 mM) | 6.5 | 15.98 | 0.10 | 17.75 | 33.00 |
| pKa = 6.04 | 7.0 | 14.88 | 0.06 | 15.92 | 26.90 |
| Phosphate (20 mM) | 6.5 | 13.38 | 0.17 | 14.81 | 36.50 |
| pKa = 7.2 | 7.0 | 14.66 | 0.25 | 16.00 | 43.50 |
| | 7.5 | 21.24 | 0.36 | 18.42 | 29.40 |
| Tris (20 mM) | 7.5 | 85.19 | 0.15 | 20.42 | 27.10 |
| pKa = 8.1 | 8.0 | 13.41 | 0.14 | 15.01 | 36.00 |

TABLE 42
Determined DLS Sizes (1-week incubation at 50° C.)

| | | Average | | Monomer | |
|---|---|---|---|---|---|
| | | Size | | Size | Monomer % |
| Buffer | pH | (d · nm) | PdI | (d · nm) | Pd |
| Succinate (20 mM) | 5.5 | 63.12 | 0.21 | 80.95 | 50.20 |
| pKa = 5.64 | 6.5 | 14.46 | 0.12 | 15.76 | 30.10 |
| Citrate (20 mM) | 5.5 | 44.20 | 0.28 | 76.39 | 49.30 |
| pKa = 6.4 | 6.0 | 16.46 | 0.25 | 15.99 | 33.00 |
| | 7.0 | 14.31 | 0.25 | 14.13 | 28.90 |
| Histidine (20 mM) | 6.5 | 98.61 | 0.44 | 185.60 | 46.10 |
| pKa = 6.04 | 7.0 | 22.52 | 0.13 | 24.23 | 30.30 |
| Phosphate (20 mM) | 6.5 | 27.91 | 0.33 | 26.08 | 38.40 |
| pKa = 7.2 | 7.0 | 14.69 | 0.20 | 15.46 | 32.00 |
| | 7.5 | 12.06 | 0.11 | 13.39 | 33.50 |
| Tris (20 mM) | 7.5 | 16.22 | 0.16 | 17.20 | 31.00 |
| pKa = 8.1 | 8.0 | 17.42 | 0.25 | 17.25 | 32.50 |

### Size Exclusion Chromatography High-Performance Liquid Chromatography (SEC-HPLC)

The SEC experiments were performed using a TOSOH G3000SW×1 (7.8×300 mm). For each sample, 90 μL was injected to achieve a column load of 90 μg (target column load of 100 g was not achievable given the low concentration of 1 mg/mL). Given the lack of historic data, the largest peak was defined as the main peak. Peaks prior to the main peak were defined as high molecular weight (HMNW) species and peaks after as low molecular weight (LMW) species. See Tables 43-44 and FIG. 41. The purity of the main peak was greater than 93% for all of the conditions tested for the 5° C. one-week incubation.

TABLE 43
SEC-HPLC Purity (1-week incubation at 5° C.)

| | | % | % | % |
|---|---|---|---|---|
| Buffer | pH | HMW | Main | LMW |
| Succinate (20 mM) | 5.5 | 1.0 | 96.6 | 2.4 |
| pKa = 5.64 | 6.5 | 1.9 | 96.0 | 2.2 |
| Citrate (20 mM) | 5.5 | 1.1 | 97.0 | 1.9 |
| pKa = 6.4 | 6.0 | 0.4 | 96.7 | 2.8 |
| | 7.0 | 1.1 | 97.4 | 1.4 |
| Histidine (20 mM) | 6.5 | 3.9 | 93.7 | 2.4 |
| pKa = 6.04 | 7.0 | 2.0 | 96.5 | 1.5 |
| Phosphate (20 mM) | 6.5 | 1.4 | 97.4 | 1.2 |
| pKa = 7.2 | 7.0 | 1.4 | 95.2 | 3.5 |
| | 7.5 | 1.3 | 95.2 | 3.5 |
| Tris (20 mM) | 7.5 | 2.2 | 96.8 | 1.0 |
| pKa = 8.1 | 8.0 | 2.0 | 95.5 | 2.5 |

TABLE 44
SEC-HPLC Purity (1-week incubation at 50° C.)

| | | % | % | % |
|---|---|---|---|---|
| Buffer | pH | HMW | Main | LMW |
| Succinate (20 mM) | 5.5 | 46.9 | 51.0 | 2.1 |
| pKa = 5.64 | 6.5 | 11.4 | 86.9 | 1.7 |
| Citrate (20 mM) | 5.5 | 26.7 | 71.9 | 1.4 |
| pKa = 6.4 | 6.0 | 6.4 | 92.0 | 1.5 |
| | 7.0 | 4.2 | 94.3 | 1.5 |
| Histidine (20 mM) | 6.5 | 44.7 | 53.7 | 1.7 |
| pKa = 6.04 | 7.0 | 41.2 | 57.8 | 1.0 |
| Phosphate (20 mM) | 6.5 | 17.2 | 81.0 | 1.8 |
| pKa = 7.2 | 7.0 | 8.8 | 89.7 | 1.5 |
| | 7.5 | 8.8 | 89.4 | 1.8 |
| Tris (20 mM) | 7.5 | 21.2 | 77.4 | 1.4 |
| pKa = 8.1 | 8.0 | 22.0 | 76.6 | 1.4 |

### Capillary Electrophoresis Sodium Dodecyl Sulfate (CE-SDS) (Reduced)

The CE-SDS experiments were performed using a Beckman Coulter PA 800 plus Capillary Electrophoresis System. Given the lack of historic data, the largest peak was defined as the main peak (peak 7). Peaks prior to the main peak were defined as LMW species and peaks after as HMNW species. See Tables 45-46 and FIG. 42. The purity of the main peak was greater than 49% for all of the conditions tested for the 5° C. one-week incubation.

TABLE 45
CE-SDS Purity (1-week incubation at 5° C.)

| | | % Purity/Impurity | | | | | | | |
|---|---|---|---|---|---|---|---|---|---|
| Buffer | pH | Peak 1 | Peak 2 | Peak 3 | Peak 4 | Peak 5 | Peak 6 | Peak 7 | Peak 8 |
| Succinate (20 | 5.5 | 0.2 | 6.9 | 8.0 | 1.3 | 5.6 | 24.8 | 52.8 | 0.4 |
| mM) | 6.5 | 0.3 | 6.9 | 7.9 | 1.2 | 5.7 | 25.0 | 52.5 | 0.4 |
| pKa = 5.64 | | | | | | | | | |
| Citrate (20 mM) | 5.5 | 0.2 | 6.9 | 7.9 | 1.2 | 5.8 | 25.0 | 52.5 | 0.4 |
| pKa = 6.4 | 6.0 | 0.2 | 6.8 | 7.9 | 1.2 | 5.8 | 24.9 | 52.8 | 0.4 |
| | 7.0 | 0.2 | 6.8 | 7.8 | 1.2 | 5.7 | 24.8 | 53.1 | 0.4 |
| Histidine (20 mM) | 6.5 | 0.2 | 7.0 | 7.7 | 1.2 | 5.4 | 25.2 | 52.8 | 0.4 |
| pKa = 6.04 | 7.0 | 0.3 | 7.1 | 7.8 | 1.1 | 5.4 | 25.1 | 52.8 | 0.4 |
| Phosphate (20 | 6.5 | 2.2 | 8.5 | 10.1 | 1.0 | 5.2 | 22.9 | 49.1 | 0.4 |
| mM) | 7.0 | 0.3 | 7.0 | 7.7 | 1.1 | 5.5 | 25.3 | 52.7 | 0.4 |
| pKa = 7.2 | 7.5 | 0.3 | 7.0 | 7.8 | 1.2 | 5.7 | 24.9 | 52.8 | 0.4 |
| Tris (20 mM) | 7.5 | 0.3 | 7.0 | 7.9 | 1.2 | 5.5 | 25.1 | 52.6 | 0.5 |
| pKa = 8.1 | 8.0 | 0.3 | 7.0 | 7.9 | 1.3 | 5.5 | 24.6 | 52.0 | 0.9 |

TABLE 46
CE-SDS Purity (1-week incubation at 50° C.)

| | | % Purity/Impurity | | | | | | | |
|---|---|---|---|---|---|---|---|---|---|
| Buffer | pH | Peak 1 | Peak 2 | Peak 3 | Peak 4 | Peak 5 | Peak 6 | Peak 7 | Peak 8 |
| Succinate (20 | 5.5 | 1.4 | 5.7 | 10.1 | 1.7 | 6.8 | 24.5 | 46.7 | 1.6 |
| mM) | 6.5 | 0.6 | 7.4 | 8.6 | 1.4 | 5.6 | 24.4 | 51.3 | 0.7 |
| pKa = 5.64 | | | | | | | | | |
| Citrate (20 mM) | 5.5 | 1.0 | 5.9 | 9.5 | 1.5 | 6.5 | 25.8 | 49.1 | 0.4 |
| pKa = 6.4 | 6.0 | 0.5 | 7.0 | 8.4 | 1.5 | 5.8 | 25.1 | 51.1 | 0.5 |
| | 7.0 | 0.6 | 7.8 | 8.9 | 1.3 | 5.7 | 24.6 | 50.5 | 0.2 |
| Histidine (20 mM) | 6.5 | 0.6 | 6.2 | 7.9 | 1.4 | 5.7 | 24.9 | 50.4 | 1.7 |
| pKa = 6.04 | 7.0 | 0.7 | 7.0 | 8.6 | 1.5 | 5.4 | 24.5 | 49.5 | 1.6 |
| Phosphate (20 | 6.5 | 2.0 | 17.0 | 18.0 | 1.3 | 5.7 | 22.0 | 32.5 | 0.6 |
| mM) | 7.0 | 0.8 | 8.1 | 9.2 | 1.5 | 5.3 | 24.5 | 49.5 | 0.9 |
| pKa = 7.2 | 7.5 | 1.1 | 9.1 | 10.0 | 1.6 | 5.5 | 22.4 | 46.5 | 2.5 |
| Tris (20 mM) | 7.5 | 0.6 | 7.3 | 8.5 | 1.5 | 5.4 | 24 | 50.1 | 1.5 |
| pKa = 8.1 | 8.0 | 0.6 | 7.5 | 8.8 | 1.5 | 5.0 | 23.7 | 49.2 | 2.4 |

### Summary and Conclusions

All the samples were analyzed by visual appearance, pH, A₂₈₀, DLS, DSF, SEC, and CE-SDS. Evaluation by visual appearance and pH showed no significant differences between the various samples. The concentrations of all the samples were slightly decreased after the 1-week incubation (a decrease of 0.1 or 0.2 mg/mL). The concentration of the succinate samples (pH 5.5 and 6.5) after a 1-week incubation at 5° C. was more decreased (from 1.1 mg/mL to 0.8 mg/mL). The CE-SDS data showed minimal variation in the main peak purity (with the exception of the phosphate pH 6.5 buffer, which had a significantly lower mean peak purity after the 50° C. 1-week incubation).

The thermal stability data indicated that low pH (5.5) and histidine buffers negatively affected the molecule. Citrate buffers (with the exception of pH 5.5) and phosphate buffers displayed the highest thermal stability (for both the 5° C. and the 50° C. 1-week incubation samples).

The light scattering data after the 5° C. 1-week incubation indicated that the citrate buffer samples (with the exception of pH 5.5) had the smallest average size with low polydispersity. The phosphate and succinate buffers similarly had a small average size with low polydispersity. After the 50° C. 1-week incubation, the citrate buffers (with the exception of pH 5.5), the phosphate buffers (with the exception of pH 6.5), and the succinate pH 6.5 buffer, all had small average sizes.

The SEC data of the 5° C. 1-week incubation samples showed minimal variation in the main peak purity. The SEC data of the 50° C. 1-week incubation samples showed a greater degree of variation, ranging from 51.0% to 94.3%. The most desirable buffers were again the citrate buffers (with the exception of pH 5.5) and the phosphate buffers

Performance of the citrate pH 6.0 buffer or the citrate pH 7.0 buffer was more desirable than the alternative buffers tested in the assays described supra.

### Excipient Analysis

The formulations listed in Table 47 were evaluated to assess the effects of various excipients and surfactants on the stability of DF-hIL-12-Fc si when buffered in 20 mM Citrate, pH 6.5. DF-hIL-12-Fc si was buffer exchanged using centrifugal ultrafiltration devices (Amicon Ultra-15 30 k MWCO) into the buffers listed in Table 47 to a target protein concentration of either 1 mg/mL or 10 mg/mL. Following the final buffer-exchange, the protein concentration was measured using UV-Visible spectroscopy (UV-Vis) with the sponsor-provided extinction coefficient (1.43 mL/cm*mg). The samples were then sterile filtered using a 0.22 μm EMD Millipore Ultrafree-CL centrifugal filter devices with Durapore membrane (Fisher Scientific Cat. #UFC40GV0S). Following sterile filtration, each formulation was handled aseptically in a laminar flow hood. The formulated samples, as specified in Table 47, were either spiked with polysorbate 80 (PS80) to a final concentration of 0.01% or were not spiked with a surfactant. The samples were then split into six equal sized aliquots. Two aliquots were stored at 2-8° C., three were stored at 50° C., and the final aliquot underwent 5 freeze thaw cycles. For the freeze thaw aliquot, the samples were frozen at −75±10° C. for at least an hour and thawed at room temperature, with visual confirmation of no ice. Both aliquots stored at 2-8° C. were pulled at 2 weeks. One aliquot was used for testing and the other aliquot was frozen at −75±10° C. A single aliquot at 50° C. was pulled at 2 weeks for testing. The other two aliquots at 50° C. were pulled at 3 weeks and frozen at −75±10° C., shown in Table 48. The testing panel is shown in Table 49. An evaluation of particulate matter by high accuracy liquid particle count (HIAC) was also executed, to evaluate surfactant.

TABLE 47
Buffers Screened for DF-hIL-12-Fc si Excipient DOE Screen

| | Buffer | | Sugar | | DF-hIL-12-Fc |
|---|---|---|---|---|---|
| Code | pH | Sugar | Conc. | Surfactant | si Conc. |
| A | 20 mM | Sucrose | High | None | 1 mg/mL |
| B | Citrate pH | | (8% w/v, | 0.01% PS80 | 1 mg/mL |
| C | 6.5 | | 233 mM) | 0.01% PS80 | 10 mg/mL |
| D | | Sucrose | Low | None | 1 mg/mL |
| | | | (4% w/v, | | |
| | | | 117 mM) | | |
| E | | Mannitol | High | None | 1 mg/ml |
| F | | | (6% w/v, | 0.01% PS80 | 1 mg/mL |
| G | | | 330 mM) | 0.01% PS80 | 10 mg/mL |
| H | | Mannitol | Low | None | 1 mg/mL |
| | | | (2% w/v, | | |
| | | | 110 mM) | | |
| I | | Sucrose | 6% w/v | None | 1 mg/mL |
| J | | Mannitol | (175 mM) | 0.01% PS80 | 1 mg/mL |
| K | | | 1% w/v | 0.01% PS80 | 10 mg/mL |
| | | | (55 mM) | | |
| L | | Sucrose | 4% w/v | None | 1 mg/mL |
| | | | (117 mM) | | |
| M | | Mannitol | 2% w/v | 0.01% PS80 | 1 mg/mL |
| N | | | (110 mM) | 0.01% PS80 | 10 mg/mL |

TABLE 48
Vial Distribution

| Vial Number | Condition | Testing |
|---|---|---|
| Vial 1 | Two weeks at 2-8° C. | Batch Tested on the |
| Vial 2 | Two weeks 50° C. | 2-8° C. sample |
| Vial 3 | 5× Freeze Thaw Cycles | only |
| | Freezing at −75 ± 10° C. | |
| | Thawing at Room Temperature | |
| Vial 4 | Two weeks at 2-8° C. | Tested as needed |
| | Then frozen at −75 ± 10° C. | |
| Vials 5 and 6 | Three weeks at 50° C. | |
| | Then frozen at −75 ± 10° C. | |

TABLE 49
Assay panel

| Test Panel | Assay Volume (μL) |
|---|---|
| Visual Appearance | In Vial |
| A₂₈₀ | 100 |
| DLS | 100 |
| SEC-HPLC, ATM-2606 | 100 |
| DSF (2-8° C. Vial 1 or | 100¹ |
| retain during sample prep)¹ | |
| HIAC² | 100 |

¹DSF performed at 1 week with material remaining from sample prep
²HIAC performed with 0.1 mL tare volume and 0.3 mL sample volume (single draw/read)

### Results

Samples of DF-hIL-12-Fc si were buffer exchanged into 14 formulations. The pH and concentration of the samples were immediately assessed. After which, the samples were aliquoted and stored at 2-8° C., 50EC, or underwent 5 freeze thaw cycles. After a two-week incubation, the samples were then assessed per the assay panel in Table 49. The untested samples were pulled and frozen as outlined previously.

Table 50 and FIGS. 43A-43B show UV-Vis Concentration Determination (time zero and 2 week samples).

TABLE 50
Concentration of DF-hIL-12-Fc si

| | | | Buffer | | | |
|---|---|---|---|---|---|---|
| | | | Exchange | 2-8° C. | 50° C. | Freeze Thaw |
| | | | Concentration | Concentration | Concentration | Concentration |
| Code | Sugar | Surfactant | (mg/mL) | (mg/mL) | (mg/mL) | (mg/mL) |
| A | 8% w/v | None | 0.90 | 0.79 | 0.80 | 0.80 |
| B | Sucrose | 0.01% PS80 | | 0.84 | 0.85 | 0.85 |
| C | | 0.01% PS80 | 10.26 | 9.94 | 10.27 | 10.20 |
| D | 4% w/v | None | 1.11 | 0.97 | 1.00 | 0.99 |
| | Sucrose | | | | | |
| E | 6% w/v | None | 0.89 | 0.81 | 0.82 | 0.82 |
| F | Mannitol | 0.01% PS80 | | 0.87 | 0.87 | 0.86 |
| G | | 0.01% PS80 | 10.61 | 10.69 | 10.91 | 10.74 |
| H | 2% w/v | None | 1.04 | 0.93 | 0.94 | 0.95 |
| | Mannitol | | | | | |
| I | 6% w/v | None | 1.03 | 0.93 | 0.92 | 0.95 |
| J | Sucrose 1% | 0.01% PS80 | | 0.98 | 0.98 | 0.98 |
| K | w/v Mannitol | 0.01% PS80 | 9.96 | 9.59 | 10.09 | 9.95 |
| L | 4% w/v | None | 1.05 | 0.92 | 0.93 | 0.94 |
| M | Sucrose 2% | 0.01% PS80 | | 0.99 | 0.98 | 0.98 |
| N | w/v Mannitol | 0.01% PS80 | 10.14 | 9.41 | 10.19 | 9.48 |

Please note: concentration = ((A₂₈₀-A₃₂₀)/1.43)*1.0 (cm path length) * Dilution Factor (if applicable)

Table 51 shows pH Determination at time zero and 2 week samples.

TABLE 51
pH values

| | | | Buffer | | | Freeze |
|---|---|---|---|---|---|---|
| | | | Exchange | 2-8° C. | 50° C. | Thaw |
| Code | Sugar | Surfactant | pH | pH | pH | pH |
| A | 8% w/v | None | 6.5 | 6.6 | 6.5 | 6.5 |
| B | Sucrose | 0.01% PS80 | | 6.5 | 6.5 | 6.5 |
| C | | 0.01% PS80 | 6.5 | 6.5 | 6.5 | 6.5 |
| D | 4% w/v | None | 6.5 | 6.6 | 6.5 | 6.5 |
| | Sucrose | | | | | |
| E | 6% w/v | None | 6.5 | 6.5 | 6.5 | 6.5 |
| F | Mannitol | 0.01% PS80 | | 6.5 | 6.5 | 6.5 |
| G | | 0.01% PS80 | 6.5 | 6.5 | 6.5 | 6.5 |
| H | 2% w/v | None | 6.5 | 6.5 | 6.5 | 6.4 |
| | Mannitol | | | | | |
| I | 6% w/v | None | 6.5 | 6.5 | 6.5 | 6.5 |
| J | Sucrose | 0.01% PS80 | | 6.5 | 6.5 | 6.5 |
| K | 1% w/v | 0.01% PS80 | 6.5 | 6.5 | 6.5 | 6.5 |
| | Mannitol | | | | | |
| L | 4% w/v | None | 6.5 | 6.5 | 6.5 | 6.5 |
| M | Sucrose | 0.01% PS80 | | 6.4 | 6.5 | 6.5 |
| N | 2% w/v | 0.01% PS80 | 6.5 | 6.5 | 6.5 | 6.4 |
| | Mannitol | | | | | |

### Visual Appearance (2 Week Samples)

All samples were colorless and clear liquids. Particulates were observed in some samples as outlined in Table 52.

TABLE 52
Appearance: Visible Particulates

| | | | 2-8° C. | 50° C. | Freeze Thaw |
|---|---|---|---|---|---|
| Code | Sugar | Surfactant | Appearance | Appearance | Appearance |
| A | 8% w/v | None | Free of visible | Some small round | Free of visible |
| | Sucrose | | particulates | visible particulates | particulates |
| B | | 0.01% PS80 | Free of visible | Free of visible | Free of visible |
| | | | particulates | particulates | particulates |
| C | | 0.01% PS80 | Free of visible | Free of visible | Free of visible |
| | | | particulates | particulates | particulates |
| D | 4% w/v | None | Free of visible | Free of visible | Some small round |
| | Sucrose | | particulates | particulates | visible particulates |
| E | 6% w/v | None | Free of visible | Free of visible | Free of visible |
| | Mannitol | | particulates | particulates | particulates |
| F | | 0.01% PS80 | Free of visible | Free of visible | Free of visible |
| | | | particulates | particulates | particulates |
| G | | 0.01% PS80 | Free of visible | Free of visible | Free of visible |
| | | | particulates | particulates | particulates |
| H | 2% w/v | None | Free of visible | Free of visible | Numerous small |
| | Mannitol | | particulates | particulates | round visible |
| | | | | | particulates |
| I | 6% w/v | None | Free of visible | Free of visible | Some small round |
| | Sucrose | | particulates | particulates | visible particulates |
| J | 1% w/v | 0.01% PS80 | Free of visible | Free of visible | Free of visible |
| | Mannitol | | particulates | particulates | particulates |
| K | | 0.01% PS80 | Free of visible | Free of visible | Free of visible |
| | | | particulates | particulates | particulates |
| L | 4% w/v | None | Free of visible | Free of visible | Some small round |
| | Sucrose | | particulates | particulates | visible particulates |
| M | 2% w/v | 0.01% PS80 | Free of visible | Free of visible | Free of visible |
| | Mannitol | | particulates | particulates | particulates |
| N | | 0.01% PS80 | Free of visible | Free of visible | Free of visible |
| | | | particulates | particulates | particulates |

### Differential Scanning Fluorimetry (DSF) (1 Week Samples)

The DSF experiments were preformed using an Unchained Labs UNcle. Samples were evaluated for their thermal stability. DF-hIL-12-Fc si thermal unfolding (Tₘ) and onset of aggregation (T_{agg}) were monitored by evaluating changes in intrinsic protein fluorescence and static light scattering (SLS at 266 nm), respectively, as a function of temperature. Samples were evaluated in triplicate, and the triplicate results were averaged. Samples were analyzed over a temperature ramp of 25° C.-95° C. at a constant linear ramp rate of 0.5° C./min. Results are shown in Table 53 and FIGS. 44A-46F.

TABLE 53
Determined DSF Tₘ₁, Tₘ₂, and T_{agg} 266
Temperatures (1 week material)

| | | | Average | Average | Average |
|---|---|---|---|---|---|
| Code | Sugar | Surfactant | Tₘ₁ (° C.) | Tₘ₂ (° C.) | T_{agg} 266 (° C.) |
| A | 8% w/v | None | 66.1 | 76.1 | 66.2¹ |
| B | Sucrose | 0.01% PS80 | 66.3 | 76.4 | 66.1 |
| C | | 0.01% PS80 | 66.4 | 77.9 | 75.6 |
| D | 4% w/v | None | 66.5 | 76.2 | 66.1 |
| | Sucrose | | | | |
| E | 6% w/v | None | 67.3 | 76.8 | 66.3 |
| F | Mannitol | 0.01% PS80 | 66.8 | 76.8 | 75.6¹ |
| G | | 0.01% PS80 | 67.1 | 77.6 | 75.9 |
| H | 2% w/v | None | 66.7 | 76.4 | 66.7 |
| | Mannitol | | | | |
| I | 6% w/v | None | 67.3 | 77.3 | 66.5 |
| J | Sucrose | 0.01% PS80 | 67.0 | 77.3 | 67.1 |
| K | 1% w/v | 0.01% PS80 | 67.1 | 78.3 | 76.0 |
| | Mannitol | | | | |
| L | 4% w/v | None | 66.9 | 77.2 | 66.5¹ |
| M | Sucrose | 0.01% PS80 | 66.8 | 76.9 | 66.7 |
| N | 2% w/v | 0.01% PS80 | 67.1 | 78.0 | 75.7 |
| | Mannitol | | | | |

¹One of the triplicates was excluded from analysis due to discrepancy in the acquired data

### Dynamic Light Scattering (DLS) (2 Week Samples)

The DLS experiments were preformed using a Malvern Zeta sizer. For each sample measurement, five consecutive scans were acquired at 25° C. The Z-average hydrodynamic diameter and polydispersity index were determined from the cumulants analysis and the Stokes Einstein equation. Polydispersity is a measurement of non-uniformity in a sample. If particles are not uniform in size, a higher polydispersity will be measured. A low polydispersity (≤0.200) indicates greater uniformity in the size of the particle. Results are shown in Tables 54-62 and in FIGS. 47A-48H.

TABLE 54
DLS Average Size

| Code | Sugar | Surfactant | 2-8° C. | 50° C. | Freeze Thaw |
|---|---|---|---|---|---|
| A | 8% w/v | None | 13.85 | 28.51 | 21.60 |
| B | Sucrose | 0.01% PS80 | 18.61 | 23.61 | 21.67 |
| C | | 0.01% PS80 | 14.75 | 30.59 | 14.66 |
| D | 4% w/v | None | 17.99 | 17.39 | 19.72 |
| | Sucrose | | | | |
| E | 6% w/v | None | 16.18 | 19.05 | 20.20 |
| F | Mannitol | 0.01% PS80 | 18.93 | 19.92 | 17.07 |
| G | | 0.01% PS80 | 14.38 | 36.92 | 13.92 |
| H | 2% w/v | None | 11.83 | 19.68 | 25.23 |
| | Mannitol | | | | |
| I | 6% w/v | None | 13.82 | 16.13 | 19.94 |
| J | Sucrose | 0.01% PS80 | 11.59 | 14.40 | 15.32 |
| K | 1% w/v | 0.01% PS80 | 14.19 | 40.96 | 13.99 |
| | Mannitol | | | | |
| L | 4% w/v | None | 24.18 | 21.08 | 19.50 |
| M | Sucrose | 0.01% PS80 | 16.03 | 17.43 | 17.20 |
| N | 2% w/v | 0.01% PS80 | 14.83 | 28.27 | 13.65 |
| | Mannitol | | | | |

| | |
|---|---|
| Starting Material | 12.25 |

TABLE 55
DLS Polydispersity

| Code | Sugar | Surfactant | 2-8° C. | 50° C. | Freeze Thaw |
|---|---|---|---|---|---|
| A | 8% w/v | None | 0.42 | 0.45 | 0.48 |
| B | Sucrose | 0.01% PS80 | 0.33 | 0.37 | 0.34 |
| C | | 0.01% PS80 | 0.15 | 0.26 | 0.14 |
| D | 4% w/v | None | 0.32 | 0.30 | 0.52 |
| | Sucrose | | | | |
| E | 6% w/v | None | 0.29 | 0.42 | 0.58 |
| F | Mannitol | 0.01% PS80 | 0.29 | 0.20 | 0.46 |
| G | | 0.01% PS80 | 0.14 | 0.24 | 0.09 |
| H | 2% w/v | None | 0.16 | 0.30 | 0.44 |
| | Mannitol | | | | |
| I | 6% w/v | None | 0.34 | 0.30 | 0.48 |
| J | Sucrose | 0.01% PS80 | 0.26 | 0.25 | 0.33 |
| K | 1% w/v | 0.01% PS80 | 0.11 | 0.22 | 0.11 |
| | Mannitol | | | | |
| L | 4% w/v | None | 0.35 | 0.35 | 0.47 |
| M | Sucrose | 0.01% PS80 | 0.32 | 0.23 | 0.40 |
| N | 2% w/v | 0.01% PS80 | 0.18 | 0.26 | 0.10 |
| | Mannitol | | | | |

| | |
|---|---|
| Starting Material | 0.09 |

TABLE 56
DLS Monomer Size

| Code | Sugar | Surfactant | 2-8° C. | 50° C. | Freeze Thaw |
|---|---|---|---|---|---|
| A | 8% w/v | None | 15.79 | 31.20 | 17.77 |
| B | Sucrose | 0.01% PS80 | 22.74 | 53.08 | 24.89 |
| C | | 0.01% PS80 | 16.76 | 44.53 | 16.87 |
| D | 4% w/v | None | 19.19 | 20.40 | 15.55 |
| | Sucrose | | | | |
| E | 6% w/v | None | 18.69 | 18.72 | 14.68 |
| F | Mannitol | 0.01% PS80 | 20.36 | 23.60 | 14.29 |
| G | | 0.01% PS80 | 16.32 | 52.95 | 15.35 |
| H | 2% w/v | None | 13.70 | 20.91 | 15.72 |
| | Mannitol | | | | |
| I | 6% w/v | None | 17.21 | 19.56 | 17.91 |
| J | Sucrose | 0.01% PS80 | 15.72 | 18.98 | 18.12 |
| K | 1% w/v | 0.01% PS80 | 16.08 | 55.60 | 15.78 |
| | Mannitol | | | | |
| L | 4% w/v | None | 17.34 | 27.61 | 17.82 |
| M | Sucrose | 0.01% PS80 | 19.60 | 22.01 | 18.15 |
| N | 2% w/v | 0.01% PS80 | 17.17 | 40.77 | 15.18 |
| | Mannitol | | | | |

| | |
|---|---|
| Starting Material | 13.47 |

TABLE 57
DLS Monomer % Pd

| Code | Sugar | Surfactant | 2-8° C. | 50° C. | Freeze Thaw |
|---|---|---|---|---|---|
| A | 8% w/v | None | 35.50 | 55.10 | 28.60 |
| B | Sucrose | 0.01% PS80 | 36.50 | 213.40 | 36.20 |
| C | | 0.01% PS80 | 36.60 | 68.00 | 36.80 |
| D | 4% w/v | None | 35.70 | 54.30 | 29.10 |
| | Sucrose | | | | |
| E | 6% w/v | None | 42.70 | 40.70 | 29.00 |
| F | Mannitol | 0.01% PS80 | 37.00 | 44.70 | 30.10 |
| G | | 0.01% PS80 | 36.20 | 67.10 | 30.70 |
| H | 2% w/v | None | 38.50 | 47.90 | 28.20 |
| | Mannitol | | | | |
| I | 6% w/v | None | 38.40 | 43.40 | 30.40 |
| J | Sucrose | 0.01% PS80 | 39.50 | 43.50 | 37.30 |
| K | 1% w/v | 0.01% PS80 | 34.50 | 59.50 | 33.60 |
| | Mannitol | | | | |
| L | 4% w/v | None | 34.90 | 66.00 | 32.20 |
| M | Sucrose | 0.01% PS80 | 42.80 | 49.10 | 36.10 |
| N | 2% w/v | 0.01% PS80 | 41.00 | 65.40 | 32.20 |
| | Mannitol | | | | |

| | |
|---|---|
| Starting Material | 31.20 |

TABLE 58
DLS Species 2 Size (200 d · nm to 1500 d · nm)

| Code | Sugar | Surfactant | 2-8° C. | 50° C. | Freeze Thaw |
|---|---|---|---|---|---|
| A | 8% w/v | None | 631.10 | 838.60 | 493.10 |
| B | | 0.01% PS80 | N/A | N/A | N/A |
| C | Sucrose | 0.01% PS80 | N/A | N/A | N/A |
| D | 4% w/v | None | N/A | 474.50 | 333.50 |
| | Sucrose | | | | |
| E | 6% w/v | None | N/A | 418.90 | 384.60 |
| F | Mannitol | 0.01% PS80 | N/A | N/A | 523.10 |
| G | | 0.01% PS80 | N/A | N/A | N/A |
| H | 2% w/v | None | N/A | 347.50 | 380.50 |
| | Mannitol | | | | |
| I | 6% w/v | None | N/A | N/A | 578.00 |
| J | Sucrose | 0.01% PS80 | N/A | N/A | N/A |
| K | 1% w/v | 0.01% PS80 | N/A | N/A | N/A |
| | Mannitol | | | | |
| L | 4% w/v | None | 971.20 | 496.60 | 600.80 |
| M | Sucrose | 0.01% PS80 | N/A | N/A | 1047.00 |
| N | 2% w/v | 0.01% PS80 | N/A | N/A | N/A |
| | Mannitol | | | | |

Starting Material

TABLE 59
DLS Species 3 Size (>1500 d.nm)

| Code | Sugar | Surfactant | 2-8° C. | 50° C. | Freeze Thaw |
|---|---|---|---|---|---|
| A | 8% w/v | None | 4548.00 | 3861.00 | 5120.00 |
| B | Sucrose | 0.01% PS80 | 4152.00 | 3025.00 | 3261.00 |
| C | | 0.01% PS80 | 4335.00 | N/A | N/A |
| D | 4% w/v | None | 1771.00 | 3873.00 | 5181.00 |
| | Sucrose | | | | |
| E | 6% w/v | None | 2538.00 | 4350.00 | N/A |
| F | Mannitol | 0.01% PS80 | 3079.00 | 4073.00 | N/A |
| G | | 0.01% PS80 | 4557.00 | N/A | N/A |
| H | 2% w/v | None | 4364.00 | 3896.00 | 5307.00 |
| | Mannitol | | | | |
| I | 6% w/v | None | 3155.00 | 2512.00 | 4862.00 |
| J | Sucrose | 0.01% PS80 | N/A | 4256.00 | 2769.00 |
| K | 1% w/v | 0.01% PS80 | N/A | N/A | N/A |
| | Mannitol | | | | |
| L | 4% w/v | None | 4428.00 | 3420.00 | 4906.00 |
| M | Sucrose | 0.01% PS80 | 3883.00 | 3969.00 | 4581.00 |
| N | 2% w/v | 0.01% PS80 | 4356.00 | N/A | N/A |
| | Mannitol | | | | |

| | |
|---|---|
| Starting Material | N/A |

TABLE 60
2-8° C. Samples Species

| Code | Sugar | Surfactant | Monomer | Species 2 | Species 3 |
|---|---|---|---|---|---|
| A | 8% w/v | None | 15.79 | 631.10 | 4548.00 |
| B | Sucrose | 0.01% PS80 | 22.74 | N/A | 4152.00 |
| C | | 0.01% PS80 | 16.76 | N/A | 4335.00 |
| D | 4% w/v | None | 19.19 | N/A | 1771.00 |
| | Sucrose | | | | |
| E | 6% w/v | None | 18.69 | N/A | 2538.00 |
| F | Mannitol | 0.01% PS80 | 20.36 | N/A | 3079.00 |
| G | | 0.01% PS80 | 16.32 | N/A | 4557.00 |
| H | 2% w/v | None | 13.70 | N/A | 4364.00 |
| | Mannitol | | | | |
| I | 6% w/v | None | 17.21 | N/A | 3155.00 |
| J | Sucrose | 0.01% PS80 | 15.72 | N/A | N/A |
| K | 1% w/v | 0.01% PS80 | 16.08 | N/A | N/A |
| | Mannitol | | | | |
| L | 4% w/v | None | 17.34 | 971.20 | 4428.00 |
| M | Sucrose | 0.01% PS80 | 19.60 | N/A | 3883.00 |
| N | 2% w/v | 0.01% PS80 | 17.17 | N/A | 4356.00 |
| | Mannitol | | | | |

TABLE 61
50° C. Samples Species

| Code | Sugar | Surfactant | Monomer | Species 2 | Species 3 |
|---|---|---|---|---|---|
| A | 8% w/v | None | 31.20 | 838.60 | 3861.00 |
| B | Sucrose | 0.01% PS80 | 53.08 | N/A | 3025.00 |
| C | | 0.01% PS80 | 44.53 | N/A | N/A |
| D | 4% w/v | None | 20.40 | 474.50 | 3873.00 |
| | Sucrose | | | | |
| E | 6% w/v | None | 18.72 | 418.90 | 4350.00 |
| F | Mannitol | 0.01% PS80 | 23.60 | N/A | 4073.00 |
| G | | 0.01% PS80 | 52.95 | N/A | N/A |
| H | 2% w/v | None | 20.91 | 347.50 | 3896.00 |
| | Mannitol | | | | |
| I | 6% w/v | None | 19.56 | N/A | 2512.00 |
| J | Sucrose | 0.01% PS80 | 18.98 | N/A | 4256.00 |
| K | 1% w/v | 0.01% PS80 | 55.60 | N/A | N/A |
| | Mannitol | | | | |
| L | 4% w/v | None | 27.61 | 496.60 | 3420.00 |
| M | Sucrose | 0.01% PS80 | 22.01 | N /A | 3969.00 |
| N | 2% w/v | 0.01% PS80 | 40.77 | N/A | N/A |
| | Mannitol | | | | |

TABLE 62
Freeze Thaw Samples Species

| Code | Sugar | Surfactant | Monomer | Species 2 | Species 3 |
|---|---|---|---|---|---|
| A | 8% w/v | None | 17.77 | 493.10 | 5120.00 |
| B | Sucrose | 0.01% PS80 | 24.89 | N/A | 3261.00 |
| C | | 0.01% PS80 | 16.87 | N/A | N/A |
| D | 4% w/v | None | 15.55 | 333.50 | 5181.00 |
| | Sucrose | | | | |
| E | 6% w/v | None | 14.68 | 384.60 | N/A |
| F | Mannitol | 0.01% PS80 | 14.29 | 523.10 | N/A |
| G | | 0.01% PS80 | 15.35 | N/A | N/A |
| H | 2% w/v | None | 15.72 | 380.50 | 5307.00 |
| | Mannitol | | | | |
| I | 6% w/v | None | 17.91 | 578.00 | 4862.00 |
| J | Sucrose | 0.01% PS80 | 18.12 | N/A | 2769.00 |
| K | 1% w/v | 0.01% PS80 | 15.78 | N/A | N/A |
| | Mannitol | | | | |
| L | 4% w/v | None | 17.82 | 600.80 | 4906.00 |
| M | Sucrose | 0.01% PS80 | 18.15 | 1047.00 | 4581.00 |
| N | 2% w/v | 0.01% PS80 | 15.18 | N/A | N/A |
| | Mannitol | | | | |

### Size Exclusion Chromatography High Performance Liquid Chromatography (SEC-HPLC) (2 Weeks Samples)

The SEC experiments were performed using a TOSOH G3000SW×1 (7.8×300 mm). The 1 mg/mL samples were injected neat, at 90 μL, to achieve a column load of 90 μg (target column load of 100 μg was not achievable given the low concentration of 1 mg/mL). The 10 mg/mL samples were injected neat, at 10 μL, to achieve a column load of 100 μg. The main peak was defined as the peak with the greatest peak area, and was consistent in retention time across all samples. Peaks eluting prior to the main peak were defined as high molecular weight (HMW) species and peaks eluting after as low molecular weight (LMW) species. The LOQ for the draft method was defined as 0.1% peak area of the total peak area.

TABLE 63
SEC-HPLC % Main

| Code | Sugar | Surfactant | 2-8° C. | 50° C. | Freeze Thaw |
|---|---|---|---|---|---|
| A | 8% w/v | None | 99.3 | 77.6 | 99.0 |
| B | Sucrose | 0.01% PS80 | 99.1 | 77.5 | 99.0 |
| C | | 0.01% PS80 | 99.3 | 74.8 | 99.2 |
| D | 4% w/v | None | 98.9 | 83.8 | 98.9 |
| | Sucrose | | | | |
| E | 6% w/v | None | 99.1 | 88.9 | 98.0 |
| F | Mannitol | 0.01% PS80 | 99.1 | 68.8 | 98.6 |
| G | | 0.01% PS80 | 99.2 | 66.3 | 99.0 |
| H | 2% w/v | None | 99.2 | 76.7 | 98.7 |
| | Mannitol | | | | |
| I | 6% w/v | None | 99.1 | 85.0 | 98.7 |
| J | Sucrose | 0.01% PS80 | 99.0 | 85.2 | 98.9 |
| K | 1% w/v | 0.01% PS80 | 99.2 | 57.4 | 99.1 |
| | Mannitol | | | | |
| L | 4% w/v | None | 99.1 | 76.6 | 98.5 |
| M | Sucrose | 0.01% PS80 | 99.1 | 78.6 | 99.0 |
| N | 2% w/v | 0.01% PS80 | 99.2 | 76.5 | 99.1 |
| | Mannitol | | | | |

| | |
|---|---|
| Starting Material | 99.4 |

TABLE 64
SEC-HPLC % HMW

| Code | Sugar | Surfactant | 2-8° C. | 50° C. | Freeze Thaw |
|---|---|---|---|---|---|
| A | 8% w/v | None | 0.7 | 20.2 | 0.9 |
| B | Sucrose | 0.01% PS80 | 0.9 | 20.4 | 1.0 |
| C | | 0.01% PS80 | 0.7 | 23.8 | 0.8 |
| D | 4% w/v | None | 0.7 | 14.6 | 1.1 |
| | Sucrose | | | | |
| E | 6% w/v | None | 0.9 | 9.3 | 2.0 |
| F | Mannitol | 0.01% PS80 | 0.9 | 29.5 | 1.4 |
| G | | 0.01% PS80 | 0.8 | 32.4 | 1.0 |
| H | 2% w/v | None | 0.7 | 21.9 | 1.3 |
| | Mannitol | | | | |
| I | 6% w/v | None | 0.9 | 13.5 | 1.3 |
| J | Sucrose | 0.01% PS80 | 1.0 | 13.3 | 1.1 |
| K | 1% w/v | 0.01% PS80 | 0.8 | 41.3 | 0.9 |
| | Mannitol | | | | |
| L | 4% w/v | None | 0.9 | 22.0 | 1.5 |
| M | Sucrose | 0.01% PS80 | 0.9 | 20.0 | 1.0 |
| N | 2% w/v | 0.01% PS80 | 0.8 | 22.4 | 0.9 |
| | Mannitol | | | | |

| | |
|---|---|
| Starting Material | 0.6 |

TABLE 65
SEC-HPLC % LMW

| Code | Sugar | Surfactant | 2-8° C. | 50° C. | Freeze Thaw |
|---|---|---|---|---|---|
| A | 8% w/v | None | <LOQ of 0.1% | 2.2 | <LOQ of 0.1% |
| B | Sucrose | 0.01% PS80 | <LOQ of 0.1% | 2.2 | <LOQ of 0.1% |
| C | | 0.01% PS80 | <LOQ of 0.1% | 1.4 | <LOQ of 0.1% |
| D | 4% w/v | None | 0.4 | 1.6 | <LOQ of 0.1% |
| | Sucrose | | | | |
| E | 6% w/v | None | <LOQ of 0.1% | 1.8 | <LOQ of 0.1% |
| F | Mannitol | 0.01% PS80 | <LOQ of 0.1% | 1.6 | <LOQ of 0.1% |
| G | | 0.01% PS80 | <LOQ of 0.1% | 1.4 | <LOQ of 0.1% |
| H | 2% w/v | None | <LOQ of 0.1% | 1.4 | <LOQ of 0.1% |
| | Mannitol | | | | |
| I | 6% w/v | None | <LOQ of 0.1% | 1.4 | <LOQ of 0.1% |
| J | Sucrose | 0.01% PS80 | <LOQ of 0.1% | 1.5 | <LOQ of 0.1% |
| K | 1% w/v | 0.01% PS80 | <LOQ of 0.1% | 1.3 | <LOQ of 0.1% |
| | Mannitol | | | | |
| L | 4% w/v | None | <LOQ of 0.1% | 1.4 | <LOQ of 0.1% |
| M | Sucrose | 0.01% PS80 | <LOQ of 0.1% | 1.5 | <LOQ of 0.1% |
| N | 2% w/v | 0.01% PS80 | <LOQ of 0.1% | 1.1 | <LOQ of 0.1% |
| | Mannitol | | | | |

| | |
|---|---|
| Starting Material | <LOQ of 0.1% |

### HIAC (2 Week Samples)

The particulate matter experiments were performed using a HIAC 9703+. For the analysis, the instrument was tared with 0.1 mL of sample prior to evaluation of 0.3 mL of sample volume. Between each run, the instrument was washed with water. In addition to the samples, the buffers and starting material were analyzed, and showed no significant number of particles. Results are shown in Tables 66-69.

TABLE 66
HIAC ≥2 μm Particles per mL

| Code | Sugar | Surfactant | 2-8° C. | 50° C. | Freeze Thaw |
|---|---|---|---|---|---|
| A | 8% w/v | None | 113.33 | 740.00 | 210.00 |
| B | Sucrose | 0.01% PS80 | 53.33 | 50.00 | 63.33 |
| C | | 0.01% PS80 | 176.67 | 193.33 | 120.00 |
| D | 4% w/v | None | 70.00 | 43.33 | 1466.67 |
| | Sucrose | | | | |
| E | 6% w/v | None | 93.33 | 83.33 | 193.33 |
| F | Mannitol | 0.01% PS80 | 73.33 | 150.00 | 73.33 |
| G | | 0.01% PS80 | 83.33 | 210.00 | 116.67 |
| H | 2% w/v | None | 40.00 | 100.00 | 5436.67 |
| | Mannitol | | | | |
| I | 6% w/v | None | 33.33 | 253.33 | 86.67 |
| J | Sucrose | 0.01% PS80 | 93.33 | 50.00 | 153.33 |
| K | 1% w/v | 0.01% PS80 | 150.00 | 280.00 | 196.67 |
| | Mannitol | | | | |
| L | 4% w/v | None | 56.67 | 76.67 | 93.33 |
| M | Sucrose | 0.01% PS80 | 283.33 | 193.33 | 60.00 |
| N | 2% w/v | 0.01% PS80 | 126.67 | 190.00 | 93.33 |
| | Mannitol | | | | |

TABLE 67
HIAC ≥5 μm Particles per mL

| Code | Sugar | Surfactant | 2-8° C. | 50° C. | Freeze Thaw |
|---|---|---|---|---|---|
| A | 8% w/v | None | 33.33 | 220.00 | 56.67 |
| B | Sucrose | 0.01% PS80 | 20.00 | 16.67 | 23.33 |
| C | | 0.01% PS80 | 53.33 | 113.33 | 50.00 |
| D | 4% w/v | None | 13.33 | 13.33 | 550.00 |
| | Sucrose | | | | |
| E | 6% w/v | None | 43.33 | 30.00 | 33.33 |
| F | Mannitol | 0.01% PS80 | 36.67 | 36.67 | 23.33 |
| G | | 0.01% PS80 | 30.00 | 46.67 | 33.33 |
| H | 2% w/v | None | 6.67 | 26.67 | 1560.00 |
| | Mannitol | | | | |
| I | 6% w/v | None | 26.67 | 56.67 | 20.00 |
| J | Sucrose | 0.01% PS80 | 33.33 | 3.33 | 36.67 |
| K | 1% w/v | 0.01% PS80 | 36.67 | 76.67 | 70.00 |
| | Mannitol | | | | |
| L | 4% w/v | None | 20.00 | 26.67 | 26.67 |
| M | Sucrose | 0.01% PS80 | 103.33 | 40.00 | 16.67 |
| N | 2% w/v | 0.01% PS80 | 40.00 | 23.33 | 33.33 |
| | Mannitol | | | | |

TABLE 68
HIAC ≥10 μm Particles per mL

| Code | Sugar | Surfactant | 2-8° C. | 50° C. | Freeze Thaw |
|---|---|---|---|---|---|
| A | 8% w/v | None | 13.33 | 60.00 | 20.00 |
| B | Sucrose | 0.01% PS80 | 6.67 | 3.33 | 6.67 |
| C | | 0.01% PS80 | 13.33 | 83.33 | 20.00 |
| D | 4% w/v | None | 6.67 | 10.00 | 66.67 |
| | Sucrose | | | | |
| E | 6% w/v | None | 10.00 | 16.67 | 13.33 |
| F | Mannitol | 0.01% PS80 | 3.33 | 10.00 | 3.33 |
| G | | 0.01% PS80 | 6.67 | 16.67 | 0.00 |
| H | 2% w/v | None | 3.33 | 6.67 | 143.33 |
| | Mannitol | | | | |
| I | 6% w/v | None | 20.00 | 26.67 | 3.33 |
| J | Sucrose | 0.01% PS80 | | | |
| K | 1% w/v | 0.01% PS80 | 13.33 | 0.00 | 0.00 |
| | Mannitol | | 16.67 | 13.33 | 33.33 |
| L | 4% w/v | None | 3.33 | 16.67 | 10.00 |
| M | Sucrose | 0.01% PS80 | 46.67 | 26.67 | 3.33 |
| N | 2% w/v | 0.01% PS80 | 10.00 | 3.33 | 20.00 |
| | Mannitol | | | | |

TABLE 69
HIAC ≥25 μm Particles per mL

| Code | Sugar | Surfactant | 2-8° C. | 50° C. | Freeze Thaw |
|---|---|---|---|---|---|
| A | 8% w/v | None | 3.33 | 0.00 | 0.00 |
| B | Sucrose | 0.01% PS80 | 0.00 | 0.00 | 0.00 |
| C | | 0.01% PS80 | 3.33 | 6.67 | 0.00 |
| D | 4% w/v | None | 0.00 | 0.00 | 0.00 |
| | Sucrose | | | | |
| E | 6% w/v | None | 0.00 | 0.00 | 0.00 |
| F | Mannitol | 0.01% PS80 | 0.00 | 0.00 | 0.00 |
| G | | 0.01% PS80 | 0.00 | 0.00 | 0.00 |
| H | 2% w/v | None | 0.00 | 6.67 | 0.00 |
| | Mannitol | | | | |
| I | 6% w/v | None | 3.33 | 6.67 | 0.00 |
| J | Sucrose | 0.01% PS80 | 3.33 | 0.00 | 0.00 |
| K | 1% w/v | 0.01% PS80 | 6.67 | 0.00 | 3.33 |
| | Mannitol | | | | |
| L | 4% w/v | None | 0.00 | 6.67 | 3.33 |
| M | Sucrose | 0.01% PS80 | 6.67 | 3.33 | 0.00 |
| N | 2% w/v | 0.01% PS80 | 0.00 | 0.00 | 6.67 |
| | Mannitol | | | | |

### Summary and Conclusions

All the samples were analyzed by visual appearance (2 week), pH (buffer exchange and 2 week), A280 (buffer exchange and 2 week), DLS (2 week), DSF (1 week), SEC (2 week), and HIAC (2 week).

The concentrations of a majority of the samples were within 10% of the target concentration at the conclusion of the buffer exchange. Both samples E (20 mM citrate, 6% mannitol, pH 6.5) and F (20 mM citrate, 6% mannitol, 0.01% PS80 pH 6.5) had concentrations of 0.89 mg/mL at the conclusion of the buffer exchange. Upon conclusion of incubation at respective conditions (2-8° C., 50° C.), 10 of the 14 formulations evaluated had concentrations consistent with their starting concentrations. All 3 samples for formulations A (20 mM citrate, 8% sucrose, pH 6.5), B (20 mM citrate, 8% sucrose, 0.01% PS80, pH 6.5), E (20 mM citrate, 6% mannitol, pH 6.5), and F (20 mM citrate, 6% mannitol, 0.01% PS80) had concentrations ranging from 0.87 mg/mL to 0.79 mg/mL.

Evaluation by pH showed no significant differences between the various samples.

Evaluation by visual appearance showed no significant differences in the color and clarity of the samples. Most of the samples, based on visual appearance, were free of visible particulates. The 50° C. sample for formulation A (20 mM citrate, 8% sucrose, pH 6.5), the freeze thaw sample for formulation D (20 mM citrate, 4% sucrose, pH 6.5), the freeze thaw sample for formulation I (20 mM citrate, 6% sucrose, 1% mannitol, pH 6.5), and the freeze thaw sample for formulation L (20 mM citrate, 4% sucrose, 2% mannitol, pH 6.5) all had some small round particulates visible. The freeze thaw sample for formulation H (20 mM citrate, 2% mannitol, pH 6.5) had numerous small round visible particulates. Notably, all 4 of these formulations lacked surfactant (0.01% P80).

Evaluation by differential scanning fluorimetry of the remaining material after buffer exchanged (stored for 1 week at 2-8° C.) showed that formulations E (20 mM citrate, 6% mannitol, pH 6.5) and I (20 mM citrate, 6% sucrose, 1% mannitol, pH 6.5) had the highest Tₘ₁ for the 1 mg/mL samples, while formulations G (20 mM citrate, 6% mannitol, 0.01% PS80, 10 mg/mL), K (20 mM citrate, 6% sucrose, 1% mannitol, 0.01% PS80 pH 6.5, 10 mg/mL), and N (20 mM citrate, 4% sucrose, 2% mannitol, 0.01% PS80, pH 6.5, 10 mg/mL) had the highest Tₘ₁ for the 10 mg/mL samples. Formulations I (20 mM citrate, 6% sucrose, 1% mannitol, pH 6.5), J (20 mM citrate, 6% sucrose, 1% mannitol, 0.01% PS80, pH 6.5), and L (20 mM citrate, 4% sucrose, 2% mannitol, pH 6.5) had the highest Tₘ₂ for the 1 mg/mL samples and formulation K (20 mM citrate, 6% sucrose, 1% mannitol, 0.01% PS80, pH 6.5, 10 mg/mL) had the highest Tₘ₂ for the 10 mg/mL samples. Formulation F (20 mM citrate, 6% mannitol, 0.01% PS80) had a significantly higher T_{agg} than the other 1 mg/mL samples. The 10 mg/mL samples were consistent with respect to T_{agg}. Overall, all samples demonstrated Tₘ₁ values >66° C.

Evaluation by SEC-HPLC of the 2 week material showed minimal variation among the 2-8° C. samples as well as the freeze thaw samples. The % Main of the 1 mg/mL 50° C. samples ranged from 68.8% to 88.9%. Formulations D (20 mM citrate, 4% sucrose, pH 6.5), E (20 mM citrate, 6% mannitol, pH 6.5), I (20 mM citrate, 6% sucrose, 1% mannitol, pH 6.5), and J (20 mM citrate, 6% sucrose, 1% mannitol, 0.01% PS80, pH 6.5) all had % Main above 80%. The % Main of the 10 mg/mL 50° C. samples ranged from 57.4% to 76.5%. Formulations C (20 mM citrate, 8% sucrose, 0.01% PS80, pH 6.5, 10 mg/mL), N (20 mM citrate, 4% sucrose, 2% mannitol, 0.01% PS80, pH 6.5, 10 mg/mL) had % Main above 70% while Formulation K (20 mM citrate, 6% sucrose, 1% mannitol, 0.01% PS80, pH 6.5, 10 mg/mL) had the lowest % Main at 57.4%.

The light scattering data indicated formulations I (20 mM citrate, 6% sucrose, 1% mannitol, pH 6.5) and J (20 mM citrate, 6% sucrose, 1% mannitol, 0.01% PS80, pH 6.5) generally had the smallest average size as well as the smallest monomer size for the 3 tested conditions. A small species was detected in some of the 1 mg/mL samples (formulations A, B, I, J, L, and M), most likely from the sucrose and mannitol. The small species was not detected in the 10 mg/mL samples, and was most likely masked by the higher concentration. Due to the automated analysis, the presence of this species complicated conclusions regarding the polydispersity of the samples. With respect to the average size, formulations I and J consistently had smaller average sizes than most of the 1 mg/mL samples for each of the 3 conditions. Formulations I and J also had a smaller monomer size for than most of the 1 mg/mL samples for the 2-8° C. condition as well as the 50° C. condition. Formulations D (20 mM citrate, 4% sucrose, pH 6.5), E (20 mM citrate, 6% mannitol, pH 6.5), F (20 mM citrate, 6% mannitol, 0.01% PS80), and H (20 mM citrate, 2% mannitol, pH 6.5) had a smaller monomer size than the rest of the 1 mg/mL samples for the freeze thaw condition. For the 10 mg/mL samples, all of the samples were largely consistent across the 3 tested conditions. The 10 mg/mL 50° C. samples displayed an appreciable increase in both average size as well as monomer size. In a number of samples a second species was detected (200 d.nm to 1200 d.nm) and in a majority of samples a third species was detected (1500 d.nm to 5500 d.nm). These larger species were primarily detected in the 1 mg/mL samples at all 3 conditions.

Evaluation by HIAC largely corroborated the visual appearance data. The 50° C. sample for formulation A (20 mM citrate, 8% sucrose, pH 6.5), the freeze thaw sample for formulation D (20 mM citrate, 4% sucrose, pH 6.5), and the freeze thaw sample for formulation H (20 mM citrate, 2% mannitol, pH 6.5) all had higher ≥2 μm particle counts, ≥5 μm particle counts, and ≥10 μm particle counts. With the exception of these three samples, the remaining samples were relatively consistent. None of the samples exceeded the USP <787> specification.

Performance of the formulation J (20 mM Citrate, 6% w/v Sucrose, 1% w/v Mannitol, 0.01% PS80, pH 6.5) was determined to be the most desirable. This buffer/excipient/surfactant combination at a higher concentration of 10 mg/mL (formulation K) did not perform well by SEC-HPLC after the 2 week incubation at 50° C. Additionally, the 1 mg/mL samples generally performed better or as well as the 10 mg/mL samples.

### Example 25: Pharmacokinetic (PK) Analysis of DF hIL12-Fc Si

DF hIL12-Fc si is a monovalent human IL12-Fc fusion protein designed to enhance the efficacy of IL12 without proportionally increasing adverse effects. DF hIL12-Fc si has a substantially longer half-life compared to rhIL12. The extended half-life of DF hIL12-Fc si enables a protracted pharmacodynamic profile without the need for frequent repeat administration and the consequent repeat spikes in IL12 exposure, which cause toxicities. The longer half-life enables significantly greater anti-tumor activity with less frequent dosing in mouse models, suggesting infrequent dosing in patients, such as once every 3 weeks (Q3W), may be efficacious and offer an acceptable safety profile.

The subcutaneous (SC) route was chosen as the most appropriate for administration of DF hIL12-Fc si because of a better pharmacokinetic (PK) profile, avoiding a spike in drug concentration at a maximum serum concentration observed post-dose (Cₘₐₓ) that may result in a better tolerability.

In vitro Pharmacology
In vitro Binding Characteristics

DF hIL12-Fc si retained the binding affinity of native IL12 and human IgG1 Fc to their respective receptors, IL12R and FcRn. In contrast, the human IgG1 Fc portion of DF hIL12-Fc si was mutated to abrogate binding to FcγRs.

In vitro Cellular Activity

To compare the potency of DF hIL12-Fc si to that of rhIL12, IFNγ production from human primary immune cells stimulated with either phytohemagglutinin (PHA) or anti-CD3 antibody was analyzed in vitro. Both DF hIL12-Fc si and rhIL12 consistently exhibited comparable potencies across IFNγ production assays with activated human PBMCs, isolated human T cells, or isolated human NK cells.

A separate in vitro study was conducted in unstimulated human PBMCs with clinical-grade DF hIL12-Fc si to evaluate for the potential of inducing cytokine release syndrome (CRS). In this study, DF hIL12-Fc si only led to a dose-dependent increase of IFNγ, consistent with expected pharmacology, but did not induce secretion of the other 7 cytokines evaluated.

In vitro Species Cross-reactivity

The in vitro activity of DF hIL12-Fc si in stimulating IFNγ release from mouse, rat, and cynomolgus monkey immune cells was analyzed in order to evaluate the appropriate species for toxicology studies.

The cynomolgus monkey (Macaca fascicularis) was selected as the only pharmacologically relevant species for the conduct of nonclinical safety studies based on: Comparisons of the amino acid sequences of IL12 across species; Binding pattern of DF hIL12-Fc si relative to IL12Rβ1 expression on cynomolgus monkey immune cell subsets in PBMCs compared to binding patterns on human PBMC subsets; and Stimulation of IFNγ release in cynomolgus monkey primary immune cells by DF hIL12-Fc si relative to that of humans.

Consistent with the literature (Schoenhaut D S, Chua A O, Wolitzky A G, Quinn P M, Dwyer C M, McComas W, et al., J Immunol. 1992; 148(11):3433-40), neither human DF hIL12-Fc si nor rhIL12 enhanced IFNγ production from mouse splenocytes or rat PBMCs.

In vivo Pharmacology
In vivo Pharmacology in Mice

As human IL12 was not functional in mouse cells, a surrogate murine IL12-Fc was generated that mirrors the human DF hIL12-Fc si drug candidate, allowing for examination of the PK/PD profile and efficacy in syngeneic mouse in vivo tumor models. Mouse IL12 is considered to have a similar expression pattern and function in mice compared to that observed in humans with native IL12 (Car 1999).

The surrogate molecule, designated DF-mIL-12-Fc si, utilizes murine IL12, in which the p35 and p40 subunits were fused to the N-termini of 2 different Fc variants. The mouse IgG2a Fc fragment was mutated to abrogate FcγR binding while retaining binding to FcRn (Schoenhaut D S, Chua A O, Wolitzky A G, Quinn P M, Dwyer C M, McComas W, et al., J Immunol. 1992; 148(11):3433-40), which has been found to be most analogous to the human Fc variant utilized in DF hIL12-Fc si (human IgG1 Fc silent).

### Ex Vivo Biological Potency of DF-mIL-12-Fc si

The potency of DF-mIL-12-Fc si (mean 50% effective concentration [EC₅₀]=2.07±0.8 pM) was comparable to that of rmIL12 (mean EC50=0.69±0.14 pM). Neither human DF hIL12-Fc si nor rhIL12 enhanced IFNγ production from mouse splenocytes, confirming a lack of species cross-reactivity.

### In Vivo Characterization and Pharmacokinetics of DF-mIL-12-Fc Si

The PK/PD profile and bioavailability of DF-mIL-12-Fc si were evaluated after a single dose administration of the molecule in BALB/c or C57BL/6 mice.

DF-mIL-12-Fc si demonstrated a protracted serum t_{1/2} of 29.85 hours, which was approximately 5 times longer than that of rmIL12 (t_{1/2}=6.05 hours). This 5-fold extended t_{1/2} of DF-mIL-12-Fc si resulted in ˜40-fold greater mediated IFNγ production (area under the concentration-time curve from the time of dosing to the time of the last observation [AUC₀₋₂₁₉ₕ]=916,654 h*pg/mL) that was more durable and sustained compared to that of relative to rmIL12 (AUC₀₋₂₁₉ₕ=20,304 h*pg/mL). IFNγ levels remained elevated for over 200 hours following a single administration of DF-mIL-12-Fc si, and this enhancement of IFNγ exposure resulted from administration DF-mIL-12-Fc si at equimolar amounts to that of the rmIL12 group, which yielded approximately the same Cₘₐₓ.

In BALB/c mice, the bioavailabilities of DF-mIL-12-Fc si were 66% and 32% when dosed IP and SC, respectively. Comparable bioavailabilities of 73% and 44% (IP and SC, respectively) were obtained in C57BL/6 mice.

Approximately 4-fold higher IFNγ secretion was observed in C57BL/6 mice compared to that of BALB/c mice (Cₘₐₓ of IFNγ was ˜25,000 and ˜6,000 μg/mL in C57BL/6 and BALB/c mice, respectively).

Importantly, the PD response, as measured by serum IFNγ levels, was similar regardless of administration route, despite the differences in bioavailability and a lower Cₘₐₓ observed following SC administration. These results suggest that the IL12-Fc format is able to achieve full PD efficacy by the SC route while exposing to only 1/10th the Cₘₐₓ as by the IV route. While certain IL12 toxicities are mediated by IFNγ and therefore may be similar following IV or SC administration, other side effects have been reported to be IFNγ independent (Leonard J P, Sherman M L, Fisher G L, Buchanan U, Larsen G, Atkins M B, et al., Blood. 1997; 90(7):2541-8) and may potentially be less pronounced following SC administration due to the lower IL12 Cₘₐₓ.

In summary, DF-mIL-12-Fc si demonstrated prolonged serum t_{1/2} and extended IFNγ production compared to rmIL12 with favorable bioavailability when dosed IP and SC in C57BL/6 and BALB/c mouse strains. However, both routes of administration yielded similar serum IFNγ levels as with IV dosing, and 4-fold higher IFNγ secretion was observed in C57BL/6 mice compared to that of BALB/c.

### DF-mIL-12-Fc si Therapeutic Index

In the B16F10 melanoma model, IL12 variants DF-mIL-12-Fc si and rmIL12 were dosed to match their IL12 serum exposure levels (DF-mIL-12-Fc si weekly and rmIL12 daily), with PD responses, tolerability, and in vivo efficacy analyzed to determine the benefit-risk profile of DF-mIL-12-Fc si in comparison to rmIL12.

Repeat SC dosing of rmIL12 in naïve C57BL/6 resulted in a profound accumulation of serum IFNγ within 6 days compared to weekly injected DF-mIL-12-Fc si; AUC IFNγ in rmIL12-treated animals was increased 2.8-4.5-fold over that in DF-mIL-12-Fc si-treated mice. In addition, subsequent doses of rmIL12 resulted in little or no IFNγ production, suggesting strong negative feedback that limits responses. In contrast, weekly administered DF-mIL-12-Fc si resulted in sustained and moderate IFNγ secretion with the second dose demonstrating a similar PD profile to the first dose. Moreover, inn B16F10 tumor-bearing C57BL/6 mice, daily dosing of 0.5 or 1 μg rmIL12 was lethal and all mice were euthanized after one week of treatment. Daily dosing of 0.25 μg rmIL12 (MTD) was less efficacious in controlling tumor progression compared to DF-mIL-12-Fc si dosed weekly at a level equimolar to 1 μg rmIL12.

These findings support that the toxicity observed in the clinical trials performed with rhIL12 were, at least partially, the consequence of a frequent administration schedule.

### Efficacy of DF-mIL-12-Fc Si Monotherapy in Mouse Tumor Models

Two different mouse models, B16F10 melanoma (derived from C57BL/6) and CT26-20.7 colon carcinoma (derived from BALB/c), were chosen to analyze DF-mIL-12-Fc si efficacy in vivo. B16F10 is a “cold” tumor model and it has been reported to be resistant to checkpoint blockade and to monoclonal antibodies with antibody-dependent cellular cytotoxicity (ADCC) function as single agents (Mosely S I, Prime J E, Sainson R C, Koopmann J O, Wang D Y, Greenawalt D M, et al., Cancer Immunol Res. 2017; 5(1):29-41). CT26 is a well characterized colon carcinoma model known to exhibit an inflammatory tumor microenvironment. CT26-20.7 is a subline of CT26 derived by transduction with a murine Tyrp1 transgene with similar growth and characteristics as the parental line.

Pharmacology studies were conducted to (1) compare in vivo activity of DF-mIL-12-Fc si and rmIL12; (2) assess dose response and frequency of DF-mIL-12-Fc si in established and large 800 mm³ tumors; (3) determine whether IP and SC administration of DF-mIL-12-Fc si mediate similar anti-tumor responses; and (4) evaluate the impact of different dosing frequencies on tumor burden.

### Efficacy of DF-mIL-12-Fc Si in CT26 Colon Carcinoma Model

In CT26-20.7 tumor-bearing BALB/c mice were treated IP once weekly for 5 weeks with DF-mIL-12-Fc si, mIgG2a isotype control, or rmIL12 at doses equimolar to 1 μg rmIL12 after mean tumor volume (MTV) reached 270 mm³. Treatment with DF-mIL-12-Fc si resulted in an increased antitumor response (p<0.0001), yielding 100% complete responses (CRs) compared to 10% CR in the rmIL12-treated group.

Examination of DF-mIL-12-Fc si dose level and frequency in CT26-20.7 tumor-bearing BALB/c mice, it was observed that a single dose of DF-mIL-12-Fc si induced 100% response rate. Although all mice responded to DF-mIL-12-Fc si treatment, responses were less durable, with 20% of the tumors progressing at a later stage. Similar results were obtained when mice were dosed once weekly for 2 weeks.

Examination of a potential dose-effect revealed a significant (p<0.05) dose-dependent titration of the antitumor effect between groups treated at 1 or 0.1 μg; however, 80% of mice treated with the 10-fold lower DF-mIL-12-Fc si dose concentration (0.1 μg) responded to treatment and survival was prolonged.

Moreover, similar antitumor efficacy against CT26-20.7 tumors was observed in tumor-bearing BALB/c mice with average tumor volumes of 230 mm³ administered DF-mIL-12-Fc si via different routes (IP or SC) QW for 7 weeks at doses equimolar to 1 μg rmIL12 (FIGS. 19A-B). These findings are consistent with published data indicating that IFNγ is a major mediator of anti-tumor efficacy, and these studies with DF-mIL-12-Fc si demonstrated similar levels of IFNγ after IV, IP or SC administration, despite differences in Cₘₐₓ with the various routes.

To analyze whether DF-mIL-12-Fc si monotherapy could induce effective immune responses against larger, end-stage tumors, CT26-20.7 tumor-bearing mice were treated 18 days post-inoculum, after tumor volumes had reached an average of 815 mm³. DF-mIL-12-Fc si was administered SC once weekly at dose levels equimolar to 1 or 2 μg of rmIL12 for 7 weeks, or equimolar to 2 μg dosed once. In this model with larger tumor volumes, a single dose of 2 μg DF-mIL-12-Fc si was sufficient to induce potent antitumor responses, with a CR rate of 80%. Moreover, weekly dosing at either 1 or 2 μg maintained tumor control, resulting in CR rates of 100% (1 μg weekly) or 90% (2 μg weekly). DF-mIL-12-Fc si was well tolerated, without clinical observations or effects on body weight, while mediating regression of the larger tumors.

Efficacy of DF-mIL-12-Fc Si Combination Therapy with PD1 Blockade in B16F10 Melanoma Model

PD1 blockade is known to have little or no efficacy against established B16F10 tumors (Mosely 2017). Combination therapy of DF-mIL-12-Fc si and PD1 blockade was performed in the B16F10 tumor model in 2 studies to analyze whether an antitumor immune response could be amplified. C57BL/6 mice were treated with DF-mIL-12-Fc si or anti-PD1 as single agents and in combination once average tumor volumes reached ˜215 mm³ or ˜200 mm³ (Study 1 and Study 2, respectively). Tumor-bearing mice were administered DF-mIL-12-Fc si IP (QW for 8 weeks in Study 1) or SC (QW for 7 weeks in Study 2) at a dose equimolar to 0.5 μg of rmIL12, and anti-PD1 was administered twice weekly IP at 200 μg (twice weekly for 19 or 13 doses, in Study 1 or 2, respectively).

While administration of DF-mIL-12-Fc si alone delayed tumor regression, combination with PD1 blockade further extended the duration of antitumor responses. Survival was extended with DF-mIL-12-Fc si in combination with PD1 blockade compared to that observed with either monotherapy. Despite the synergistic efficacy, the regimen of DF-mIL-12-Fc si and anti-PD1 combination therapy appeared to be well tolerated by B16F10 tumor-bearing mice. There were no signs of additive or synergistic toxicity with the combination of DF-mIL-12-Fc si and anti-PD1, and there were neither clinical observations nor effects on body weight.

In vivo Pharmacology in Monkeys

DF hIL12-Fc si was administered IV (1.9 to 40 μg/kg) or SC (1 to 20 μg/kg) in cynomolgus monkeys in the nonclinical toxicology studies. After administration, a substantial secondary peak of PD markers IFNγ and interferon gamma-inducible protein 10 (IP-10) followed, which was dose dependent.

In non-GLP and GLP studies, monkeys treated with DF hIL12-Fc si demonstrated a generally dose-dependent increase in IFNγ, with peak levels occurring 48 to 72 hrs post-dose. IP10 was also similarly increased. In head to head non-GLP comparisons with rhIL-12 and DF hIL12-Fc si at equimolar dose levels, DF hIL12-Fc si generally caused a greater increase in IFNγ that lasted longer than the IFNγ response produced by rhIL12. DF hIL12-Fc si produced IFNγ responses that were still detectable 120-168 hrs post-dose, while molar equivalent rhIL12 IFNγ returned to baseline at similar timepoints. The more durable IFNγ response of DF hIL12-Fc si is most likely due to DF hIL12-Fc si's prolonged t_{1/2} in comparison to rhIL12. IP10 was not assessed in the non-GLP head to head comparisons with rhIL12.

In the GLP study, peak IFNγ levels were generally similar at ≥8 μg/kg SC and 12 μg/kg IV, though there was some individual animal variability within groups. Some attenuation of the IFNγ and IP10 response was observed after repeat dosing, but some monkeys in the GLP study demonstrated significant IFNγ response to the first and second dose. After the third dose in the GLP study, there was minimal to no increase in IFNγ either due to attenuation or possible impact of anti-drug antibodies (ADA). IP10 response after the third dose of DF hIL12-Fc si was more significant than the IFNγ response, possibly reflecting greater t_{1/2} of IP10.

### Secondary Pharmacology

### Binding to FcγR

The potential for DF hIL12-Fc si to bind to FcγRs was evaluated by surface plasmon resonance (SPR). The Biacore™ 8K SPR system was used to evaluate binding of DF hIL12-Fc si to recombinant human (CD64, CD32a H131 and R131 alleles, CD16a V158 and F158 alleles, CD32b, CD16b) and cynomolgus (CD64 and CD16) receptors that were captured on the chip via site-specific biotinylation. Trastuzumab, a well-established IgG1 biologic drug, was used as an isotype-specific experimental control. Qualitative assessment of the data concluded that DF hIL12-Fc si did not demonstrate meaningful binding to any of the FcγRs tested. In contrast, titration of the IgG1 control trastuzumab at the same receptor-specific, physiologically relevant concentrations demonstrated a full range of dose-dependent binding across FcγRs, as expected.

Furthermore, the human IgG1 Fc domain is known to bind C1q, a component of the classical complement cascade which mediates complement-dependent cytotoxicity (CDC) (Idusogie 2000). To confirm that DF hIL12-Fc si does not elicit CDC, human PBMCs stimulated with PHA for 3 days were incubated with 5% human complement serum in the presence of DF hIL12-Fc si ranging from 0.0823 to 20 nM. The addition of serum did not trigger CDC. In contrast, anti-MHC1 antibody (used as a positive control) induced complement serum-dependent death of T cells.

### C-Reactive Protein as a Surrogate for IFNγ Secretion

C-reactive protein (CRP) is a marker of early phase inflammation used to monitor patients that are suspected of having severe infection. Increased CRP levels are used in the cynomolgus studies as a surrogate for the measurement of the secretion of IFN gamma, which is much more challenging to use as a clinical biomarker because of its short half-life. Based on these studies, the measurement of CRP represents a more reliable biomarker for detecting the PD activity of DF hIL12-Fc si in clinical settings.

### Binding to FcRn

An evaluation of the potential for DF hIL12-Fc si to bind to cynomolgus monkey and human FcRn showed that, as expected, human and cynomolgous monkey FcRn binding was not affected by FcγR-silencing mutations. DF hIL12-Fc si and the IgG1 isotype control trastuzumab were comparable (<1.5× difference) in their binding affinity values for cynomolgus monkey and human FcRn at pH 6.0. Likewise, both molecules were similar in their lack of quantifiable binding at the concentrations tested at pH 7.4.

### Safety Pharmacology

Safety pharmacology endpoints (e.g., cytokine assessment, body temperature, respiration rate, blood pressure, heart rate, ECG assessments, and FOB assessments) were incorporated in the GLP 3-week repeat-dose toxicology study in cynomolgus monkeys.

After administration of DF hIL12-Fc si SC up to 20 μg/kg or IV at 12 μg/kg to monkeys QW for 3 weeks, there were no DF hIL12-Fc si-related effects on body temperature, blood pressure, or the central nervous system (as measured by FOB assessments), respiratory system (as measured by respiratory rate), or cardiovascular system (as measured by ECGs and heart rate).

In the GLP, 3-week repeat-dose toxicology study, IFNγ and IP10 were robustly increased after administration of DF hIL12-Fc si, which is consistent with its expected pharmacology. There were also sporadic and minimal increases of IL-6 in some DF hIL12-Fc si-treated monkeys. In non-GLP studies in monkeys, in addition to expected IFNγ and IP10 increases, there were also minimal and primarily transient increases in IL6, macrophage inflammatory protein (MIP) MIP-1α, MIP-1β, and thymus- and activation-regulated chemokine (TARC), while other measured cytokines were unaffected. These in vivo results in monkeys are consistent with the in vitro results of the cytokine release assay in unstimulated human PBMCs, in which only a concentration-dependent increase in IFNγ was observed, and other cytokines were unaffected. Therefore, DF hIL12-Fc si has a low potential for CRS, but select cytokines (e.g., IFNγ, IP-10) are expected to increase because of the expected pharmacology of DF hIL12-Fc si.

### Pharmacokinetics and Drug Metabolism in Animals

### Overview

The toxicokinetic (TK) profile of DF hIL12-Fc si was investigated after single, repeat, and/or crossover SC (21 to 20 μg/kg) and IV (1.9 to 40 μg/kg) administration to cynomolgus monkeys in 4 non-GLP toxicology studies and 1 GLP toxicology study. Plasma TK was evaluated using DF hIL12-Fc si concentrations obtained by both enzyme-linked immunosorbent assay (ELISA) (measuring IL-12p70 of DF hIL12-Fc si by detecting each IL 12 subunit) and MSD (measuring IL12p40 and Fc of DF hIL12-Fc si). Data derived from the qualified ELISA method were the preferred source for exposure assessment of DF hIL12-Fc si. An anti-drug antibody (ADA) method was also developed to detect anti-DF hIL12-Fc si antibodies in cynomolgus monkey serum after SC dosing; this method was validated for use in the GLP 3-week toxicology study.

The predominant TK profile of DF hIL12-Fc si after SC or IV administration was characterized by dose-independent (linear) kinetics, although small animal numbers in non-GLP studies and ADA may have contributed to observed variability. There did not appear to be an overall sex-related difference in the plasma TK of DF hIL12-Fc si.

In the non-GLP studies, after SC administration, bioavailability was approximately 60% based on ELISA data. The estimated t_{1/2} ranged from 12.4 to 56.4 hours across individual animals after single or repeat SC administration in 4 non-GLP studies. The tₘₐₓ ranged from 4 to 36 hours across individual animals after SC administration, was most commonly 8 hours post-dose. Across individual animals after IV administration, the estimated t_{1/2} ranged from 16.2 to 82.4 hours, with tₘₐₓ ranging from 0.25 to 1 hour post-dose.

The TK profile of DF hIL12-Fc si was confirmed in the GLP toxicology study. In general, sex-related differences in DF hIL12-Fc si mean Cₘₐₓ, area under the concentration-time curve from the time of dosing to 24 hours post-dose (AUC₀₋₂₄), and AUC₀₋₁₆₈ values were less than 2-fold. After SC dosing, exposure, as assessed by DF hIL12-Fc si mean Cₘₐₓ and AUC₀₋₁₆₈, generally increased with increasing dose level from 4 μg/kg DF hIL12-Fc si. The increases in mean Cₘₐₓ and AUC₀₋₁₆₈ were dose proportional. No accumulation of DF hIL12-Fc si was observed after multiple doses of DF hIL12-Fc si. Subcutaneous bioavailability of DF hIL12-Fc si was approximately 40%. Mean t_{1/2} for SC administration ranged from 17.5 to 35.8 hours on Days 1 and 15, while mean t_{1/2} for IV administration was 22.2 hours on Day 1 and 45.3 hours on Day 15.

In the single-dose non-GLP SC study in monkeys, ADA to DF hIL12-Fc si was demonstrated by Day 8, with confirmed ADA up to Day 22; however, overall titers of ADA remained relatively low and close in value to low positive control. In another non-GLP study, an initial SC dose followed by an IV dose demonstrated a lower than expected IV exposure profile, which may be explained by ADA, although titers were not measured. Overall, data from non-GLP studies suggest that the SC route in monkeys may be more prone to ADA development than the IV route, which was also confirmed in the GLP study. For example, 9 of 10 monkeys at 20 μg/kg SC and 3 of 10 monkeys at 12 μg/kg IV developed ADA by Day 15 in the GLP study. The majority of confirmed ADA samples were seen on Day 15 before the third dose, and maximum concentrations (Cₘₐₓ) was impacted in some animals, suggesting the presence of neutralizing antibodies. ADA impacted exposures of individual monkeys in both the non-GLP and GLP studies, but suitable exposure was achieved for a long enough duration to confidently define the toxicology profile of DF hIL12-Fc si. However, ADA in monkeys is not predictive of immunogenicity in humans. For these reasons, the DF hIL12-Fc si-001 first-in-human (FIH) study will evaluate serum titers of anti-DF hIL12-Fc si antibodies throughout the study.

Additionally, the pharmacologic response to DF hIL12-Fc si, as measured by IFNγ response, was variable across individual animals, with no apparent sex-related differences, but showed some dose-dependency when comparing tolerated doses. The peak IFNγ response ranged from 3 to 5 days post-dose across animals in the non-GLP and GLP studies. In the GLP study, the IFNγ response was attenuated with repeat dosing, although a small percentage of animals demonstrated an IFNγ response after both the first and second doses. Additionally, dose-dependent increases in IP-10 were detectable after the first and third doses, with attenuation. The route of administration did not appear to impact the timing of the peak pharmacologic response.

Studies to evaluate metabolism and excretion have not been conducted because these studies, which are routinely conducted for small molecule drugs, are not considered necessary or useful for biologics such as monoclonal antibodies. Dedicated studies have not been conducted to evaluate drug-drug interactions (DDIs) because there is no reason to believe that DF hIL12-Fc si, an IL12-Fc fusion protein, is metabolized by cytochrome P450 (CYP) enzymes. Therefore, it is unlikely that a small molecule that inhibits or induces CYP enzymes could impact the PK of DF hIL12-Fc si, and thus, the PK DDI potential for DF hIL12-Fc si is considered to be low.

### Absorption and Pharmacokinetics

### Single-Dose Pharmacokinetics

Exposures after administration of a single dose of DF hIL12-Fc si as part of the single- or repeat-dose toxicology studies were informative for acute toxicity assessment. Across studies, DF hIL12-Fc si was tolerated up to 20 μg/kg as a single SC dose. The single-dose IV maximum tolerated dose (MTD) was ≤19 μg/kg in females and ≤20 μg/kg in males; a single IV dose ≥20 or ≥40 μg/kg in females or males, respectively, resulted in early euthanasia 8 days after DF hIL12-Fc si administration.

Day 1 exposures (as measured by Cₘₐₓ and area under the concentration-time curve from the time of dosing to the time of the last quantifiable sample [AUC₀₋ₜ]), as well as tₘₐₓ and t_{1/2}, derived from ELISA data obtained from the single- and repeat-dose toxicology studies are summarized in Table 70.

TABLE 70
Comparison of Day 1 Plasma Toxicokinetics in Cynomolgus Monkeys

| | Dose | | | | | | | | | | |
|---|---|---|---|---|---|---|---|---|---|---|---|
| Study (GLP | Level | Route of | | Cₘₐₓ (pg/mL) | | AUC₀₋ₜ (h*pg/mL)^{a} | | tₘₐₓ (h) | | t_{1/2} (h) | |
| Status) | (μg/kg) | Administration | n | M | F | M | F | M | F | M | F |
| RW11CN | 1 | SC | 2/sex | 2010 | 1380 | 85,100^{b} | 61,600^{b} | 8^{c} | 8^{c} | 13.8^{d} | N/A |
| (Non-GLP) | 2 | SC | 2/sex | 3900 | 1670 | 161,000^{b} | 149,000 | 6^{c} | 8^{c} | 17.3 | 42.4 |
| | 4 | SC | 2/sex | 5410 | 8010 | 425,000^{e} | 515,000^{e} | 22^{d} | 6^{c} | 32.5^{d} | 37.8 |
| TD36MM | 4 | SC | 2/sex | 8860 | 10,500 | 562,000^{e} | 590,000^{e} | 22^{c} | 22^{c} | 40.2 | 35.4 |
| (Non-GLP) | 8 | SC | 2/sex | 14,000 | 20,200 | 783,000^{e} | 1,550,000^{e} | 8^{c} | 24^{c} | 33.9 | 29.6^{d} |
| NF37DV | 4 | SC | 3/sex | 11,800 | 10,300 | 480,000 | 426,000 | 8^{c} | 8^{c} | 19.5 | 17.5 |
| (GLP) | 8 | SC | 5/sex | 16,900 | 12,800 | 1,080,000 | 720,000 | 8^{c} | 8^{c} | 18.5 | 28.0 |
| | 20 | SC | 5/sex | 25,700 | 32,900 | 2,390,000 | 2,490,000 | 24^{c} | 48^{c} | 31.9 | 35.8 |
| | 12 | IV | 5/sex | 147,000 | 189,000 | 3,240,000 | 4,440,000 | 1^{c} | 1^{c} | 25.6 | 18.8 |
| QW56LH | 1.9 | IV | 1/sex | 53,600 | 47,900 | 1,450,000 | 921,000 | 0.25 | 0.25 | 30.5 | 17.4 |
| (Non-GLP) | 19 | IV | 1/sex | 570,000 | 502,000 | 20,700,000 | 15,100,000 | 0.25 | 0.25 | 26.4 | 19.0 |
| DQ81GX | 20^{f} | IV | 2/sex | 498,000 | 572,000 | 14,800,000^{g} | 16,900,000 | 0.25^{c} | 0.25^{c} | 70.0 | 22.9 |
| (Non-GLP) | 40^{h} | IV | 2/sex | 1,120,000 | 994,000 | 37,300,000 | 40,300,000 | 0.25^{c} | 0.25^{c} | 22.7 | 37.3 |

Sources: RW11CN, TD36MM, QW56LH, DQ81GX, and NF37DV.
Cₘₐₓ: maximum plasma concentration; AUC₀₋ₜ: area under the concentration-time curve from the time of dosing to the time of the last quantifiable sample.
Note:
All TK exposures presented here were obtained using DF hIL12-Fc si plasma concentrations measured using a qualified ELISA method (targeting p70 of IL12).
^{a}AUC exposures were based on concentrations quantified through 168 hours, unless otherwise noted.
^{b}AUC₀₋₇₂.
^{c}Median value.
^{d}n = 1.
^{e}AUC₀₋₂₄₀.
^{f}DF hIL 12-Fc si was not tolerated in females at 20 μg/kg, resulting in euthanasia on Day 8.
^{g}AUC₀₋₃₃₆.
^{h}DF hIL 12-Fc si was not tolerated in males and females at 40 μg/kg, resulting in euthanasia on Day 8.

Comparison of Acute Toxicity and Exposures Across all Toxicology Studies after Single-Dose Administration

Tolerability assessment and exposures after administration of the initial, first dose in repeat-dose toxicity studies are informative for acute toxicity assessment (Studies TD36MM, QW56LH, DQ81GX, and NF37DV). The single-dose IV MTD was ≤19 μg/kg in females and ≤20 μg/kg in males; a single IV dose ≥20 or ≥40 μg/kg in females or males, respectively, resulted in early euthanasia 8 days after DF hIL12-Fc si administration (Study DQ81GX). The exposures at 19 and 20 μg/kg IV overlapped with similar IFNγ pharmacology, suggesting animal-to-animal variation in the immune system in response to DF hIL12-Fc si treatment, which may have led to differential tolerability. This is consistent with the known variability of the immune system as a target organ (Brodin P, Davis M M., Nat Rev Immunol. 2017; 17(1):21-9).

### Repeat-Dose Pharmacokinetics

Toxicokinetics after Repeat Subcutaneous Intravenous Administration of DF hIL12-Fc Si Via a Crossover Design (Studies TD36A&I and DX81GX)

In a non-GLP study (Study TD36MM), cynomolgus monkeys were administered DF hIL12-Fc si, first SC and then IV, with a 14-day washout period after each dose, with samples for TK collected through 336 hours post-dose.

After SC administration, the systemic exposure of both male and female monkeys to DF hIL12-Fc si appeared to be characterized by dose-independent (linear) kinetics over the dose range of 4 to 8 μg/kg. The systemic exposure to DF hIL12-Fc si tended to be slightly higher in females than in males.

After IV administration to monkeys who had previously received DF hIL12-Fc si by the SC route, the systemic exposure of male and female monkeys to DF hIL12-Fc si appeared to be characterized by dose-dependent (nonlinear) kinetics over the dose range of 2 to 4 μg/kg, such that increasing the dose of DF hIL12-Fc si above 2 μg/kg resulted in a lower systemic exposure than was predicted from a linear relationship in males, but a higher systemic exposure in females. These results would be consistent with the production of ADA to DF hIL12-Fc si in the 2 males and 1 female before the IV dose was administered. Consequently, the results after IV administration must be interpreted with caution.

SC bioavailability of DF hIL12-Fc si appeared to be 60% to 70% (based on 4 animals); however, it should be noted that anomalously high estimates of bioavailability (omitted from the range presented here) were observed in some animals, consistent with the production of ADA affecting the exposure after IV administration, although ADA was not measured on this study.

In another non-GLP study (DQ81GX), cynomolgus monkeys were administered DF hIL12-Fc si first via IV bolus and then SC, with a 14-day washout period between doses. A separate group of animals received rhIL12 with the same routes and frequency of administration to allow for comparison. Blood samples for TK analysis were obtained through 240 hours post-dose. Plasma concentrations of DF hIL12-Fc si and rhIL12 were measured by qualified ELISA (targeting p70 of IL12 [ie, measuring rhIL12 and DF hIL12-Fc si]) and MSD (targeting p40 and Fc of DF hIL12-Fc si [ie, measuring DF hIL12-Fc si and not measuring rhIL12]) methods. Because of the toxicity observed after IV administration of DF hIL12-Fc si, the TK profile for SC administration was characterized only for DF hIL12-Fc si (males only) and rhIL12.

Plasma concentrations of DF hIL12-Fc si after IV administration were lower when measured by the MSD method (as to be expected, given the ELISA method detects the IL12 heterodimer). While the Cₘₐₓ values derived from the MSD data were approximately 54% lower than those derived from the ELISA method, the AUC₀₋ₜ values were similar to those derived from the ELISA method in males and 18% higher in females. The AUC₀₋ₜ values of DF hIL12-Fc si in female monkeys were similar to those in males after IV administration when measured using the ELISA method but were slightly higher when measured by the MSD method. The relationships between AUC₀₋ₜ and dose level after IV administration (based on ELISA data) indicated exposures increased in a slightly greater than dose-proportional manner over the dose range of 20 to 40 μg/kg. The tₘₐₓ after IV administration was 0.25 hours post-dose (the first sampling time), as was expected, whereas the tₘₐₓ ranged from 4 to 24 hours across individual animals after SC administration.

The t_{1/2} varied quite widely (ranging from 16.2 to 82.4 hours across individual animals) after IV administration at 20 or 40 μg/kg, but could only be estimated adequately for 1 animal after SC administration (14.9 hours based on ELISA data and 62.0 hours based on MSD data). Subcutaneous bioavailability of DF hIL12-Fc si at 20 μg/kg appeared to be variable, with a mean value of 53.5% (range of 38.8% to 68.2%) based on ELISA data and 89.0% (range of 62.8% to 115.3%) based on MSD results.

The Cₘₐₓ and AUC₀₋ₜ values of rhIL12 in female monkeys were similar to those of males after IV administration; however, the Cₘₐₓ and AUC₀₋ₜ values were lower than those in males after SC administration. The tₘₐₓ after IV administration was also 0.25 hours post-dose, whereas the tₘₐₓ was 8 hours post-dose after SC administration. The t/ranged from 9.1 to 17.5 hours across individual animals after IV administration and 18.9 to 22.9 hours after SC administration. Subcutaneous bioavailability of rhIL12 at 10 μg/kg was approximately 31% in males and 18% in females (overall range of 18.0% to 35.2%).

Toxicokinetics after Repeat Intravenous Administration of DF hIL12-Fc Si (Study QW56LH)

In a non-GLP study, cynomolgus monkeys were administered DF hIL12-Fc si, or rhIL12 via IV bolus on Days 1 and 8, with samples for TK collected through 168 hours postdose. Plasma concentrations were measured by both qualified ELISA (targeting p70 of IL12 [ie, measuring rhIL12 and DF hIL12-Fc si]) and MSD (targeting p40 and Fc of DF hIL12-Fc si [ie, measuring DF hIL12-Fc si and not measuring rhIL12]) methods.

After repeated IV bolus administration of DF hIL12-Fc si, there was no accumulation of DF hIL12-Fc si. The AUC₀₋₁₆₈ of DF hIL12-Fc si increased in an approximately dose-proportional manner over the dose range of 1.9 to 19 μg/kg on Days 1 and 8 in males, but tended to increase in a greater than dose-proportional-manner in females. At 19 μg/kg, the female AUC₀₋₁₆₈ was approximately 1.8-fold higher than that predicted from a linear relationship. The AUC₀₋₁₆₈ of DF hIL12-Fc si in females was generally similar to that in males, although the female AUC₀₋₁₆₈ derived from the MSD data appeared to be lower than that of the male at 1.9 μg/kg on both Days 1 and 8.

The terminal t_{1/2} could not be estimated adequately for all animals but, where it could be estimated was in the range of 17.4 to 30.5 hours and generally appeared to be independent of dose and sex. The plasma clearance of DF hIL12-Fc si was low, and the volume of distribution was slightly lower than the blood volume (73.4 mL/kg) and considerably lower than the volume of total body water (693 mL/kg) (Davies B, Morris T., Pharm Res. 1993; 10(7):1093-5).

After repeated IV bolus administration of rhIL12, there was no accumulation of rhIL12. The AUC₀₋₁₆₈ of rhIL12 generally increased in an approximately dose-proportional manner over the dose range of 1 to 10 μg/kg on Days 1 and 8, but tended to increase in a greater than dose-proportional manner in males on Day 1. At 10 μg/kg, the male AUC₀₋₁₆₈ on Day 1 was approximately 1.8-fold higher than that predicted from a linear relationship. There did not appear to be any differences in the systemic exposure of males and females to rhIL12. The terminal t_{1/2} (7.2 to 17.0 hours) was shorter than that of DF hIL12-Fc si, the plasma clearance was low, and the volume of distribution was similar to the blood volume and considerably lower than the volume of total body water.

The shorter half-life of rhIL12 compared to that of DF hIL12-Fc si explains the greater (˜4.5× to 7.4×) AUC₀₋₁₆₈ observed with DF hIL12-Fc si.

TABLE 71
Mean Plasma Toxicokinetics of DF hIL12-Fc si After Subcutaneous or Intravenous
Administration to Cynomolgus Monkeys in a 3-Week Study (Study NF37DV)

| Dose | | | | | | | | | | |
|---|---|---|---|---|---|---|---|---|---|---|
| Level | Cₘₐₓ | | AUC₀₋₁₆₈ | | tₘₐₓ^{a} | | t_{1/2} | | F | |
| (μg/kg)/ | (pg/mL) | | (h*pg/mL) | | (h) | | (h) | | (%) | |
| Route | M | F | M | F | M | F | M | F | M | F |

Day 1^{b}

| | | | | | | | | | | | |
|---|---|---|---|---|---|---|---|---|---|---|---|
| 4 | (SC) | 11,800 | 10,300 | 480,000 | 426,000 | 8.0 | 8.0 | 19.5 | 17.5^{c} | 37.5 | 33.3 |
| 8 | (SC) | 16,900 | 12,800 | 1,080,000 | 720,000 | 8.0 | 8.0 | 18.5 | 28.0 | 42.2 | 28.1 |
| 20 | (SC) | 25,700 | 32,900 | 2,390,000 | 2,490,000 | 24.0 | 48.0 | 31.9 | 35.8^{d} | 37.4 | 38.9 |
| 12 | (IV) | 147,000 | 189,000 | 3,240,000 | 4,440,000 | 1.0 | 1.0 | 25.6 | 18.8 | NA | NA |

Day 15

| | | | | | | | | | | | |
|---|---|---|---|---|---|---|---|---|---|---|---|
| 4 | (SC) | 9580^{c} | 7240^{c} | NR | NR | 8.0^{c} | 8.0^{c} | NR | NR | NA | NA |
| 8 | (SC) | 17,300^{d} | NA | 451,000^{e} | NR | 8.0^{d} | NR | NR | NR | NA | NA |
| 20 | (SC) | 38,000^{e} | 37,300^{e} | 2,650,000^{e} | NR | 8.0^{e} | 24.0^{e} | 23.9^{e} | NR | NA | NA |
| 12 | (IV) | 155,000^{f} | 173,000^{d} | 3,400,000^{f} | 2,280,000^{d} | 1.0^{f} | 1.0^{d} | 24.2^{c} | 59.8^{e} | NA | NA |

Source: Study NF37DV.
AUC₀₋₁₆₈: area under the concentration-time curve from the time of dosing to 168 hours post-dose; ADA: anti-drug antibodies; Cₘₐₓ: maximum plasma concentration; ELISA: enzyme-linked immunosorbent assay; F: female(s); M: male(s); NA: not applicable; NR: not reported due to insufficient number of ADA-negative animals; TK: toxicokinetic(s).
Note:
All TK parameters were derived from concentrations quantified by validated ELISA. Number of animals per sex per group are denoted in the footnotes. On Day 15, exposures are presented only for animals that did not have ADA, even though there was quantifiable exposure in these ADA-positive animals. Because DF hIL 12-Fc si is intended to be dosed once every 3 weeks in patients, Day 1 exposures from monkey studies were used as the best comparison to the intended human dosing schedule.
^{a}Median values;
^{b}n = 5/sex/group on Day 1, with the exception of 4 μg/kg SC (n = 3/sex);
^{c}n = 2/sex;
^{d}n = 3/sex;
^{e}n = 1/sex;
^{f}n = 5/sex.

In general, sex-related differences in DF hIL12-Fc si mean Cₘₐₓ, AUC₀₋₂₄, and AUC₀₋₁₆₈ values were less than 2-fold. After SC dosing, exposure, as assessed by DF hIL12-Fc si mean Cₘₐₓ and AUC₀₋₁₆₈, generally increased with increasing dose level from 4 μg/kg DF hIL12-Fc si. The increases in mean Cₘₐₓ and AUC₀₋₁₆₈ were dose proportional. No accumulation of DF hIL12-Fc si was observed after multiple doses of DF hIL12-Fc si in monkeys. Subcutaneous bioavailability of DF hIL12-Fc si was approximately 40%. Mean t₂ for SC administration ranged from 17.5 to 35.8 hours on Days 1 and 15, while mean t_{1/2} for IV administration was 22.2 hours on Day 1 and 45.3 hours on Day 15.

The incidence of ADA induction to DF hIL12-Fc si was 0% (0 out of 10) at 0 μg/kg, 33% (2 out of 6) at 4 μg/kg SC, 80% (8 out of 10) at 8 μg/kg SC, 90% (9 out of 10) at 20 μg/kg SC, and 30% (3 out of 10) at 12 μg/kg IV. Plasma concentrations of DF hIL12-Fc si in the ADA-positive animals on Day 15 were overall lower, but generally still quantifiable, than those in the ADA-negative animals. The effect of ADA was variable, with plasma concentrations in ADA-positive animals ranging from being similar to those in ADA-negative animals in some cases, to being markedly lower in others. However, this indicates that, in general, the ADA-positive animals were still exposed to DF hIL12-Fc si on Day 15. Thus, ADA did not negatively impact the toxicology study interpretation, as there was adequate exposure during most of the dosing period.

### Bioavailability

After SC administration in cynomolgus monkeys in non-GLP Studies TD36MM and DQ81GX, DF hIL12-Fc si generally demonstrated bioavailability of approximately 60%, although some variability was observed. Although not confirmed, it is believed that ADA development may have impacted the second IV dose in Study TD36MM, which subsequently would have impacted bioavailability calculations; therefore, these values were excluded when considering the overall average bioavailability across animals. Table 72 presents the bioavailability across animals within these studies.

In GLP Study NF37DV, SC bioavailability of DF hIL12-Fc si, based on AUC₀₋₁₆₈, was in the range of 18.2% to 52.8% across individual male and female animals, with mean values of 35.4%. 35.1% and 38.2% after the first dose at 4, 8, and 20 μg/kg SC, respectively.

TABLE 72
Assessment of DF hIL12-Fc si Bioavailability

| Study | Route of | Animal | | Dose | Bioavailability |
|---|---|---|---|---|---|
| Number | Administration | ID | Sex | (μg/kg) | (SC/IV AUC₀₋ₜ/dose) |
| TD36MM | SC then IV | 1200 | Male | 4-SC, 2-IV | 67% |
| TD36MM | SC then IV | 1201 | Male | 4-SC, 2-IV | 59% |
| TD36MM | SC then IV | 1700 | Female | 4-SC, 2-IV | 386%ª |
| TD36MM | SC then IV | 1701 | Female | 4-SC, 2-IV | 64% |
| TD36MM | SC then IV | 2200 | Male | 8-SC, 4-IV | 536%ª |
| TD36MM | SC then IV | 2201 | Male | 8-SC, 4-IV | 604%ª |
| TD36MM | SC then IV | 2700 | Female | 8-SC, 4-IV | 62% |
| TD36MM | SC then IV | 2701 | Female | 8-SC, 4-IV | 479%ª |
| DQ81GX | IV then SC | 2170 | Male | 20-IV, 20-SC | 39% |
| DQ81GX | IV then SC | 2171 | Male | 20-IV, 20-SC | 68% |
| NF37DV | SC | 2360 | Male | 4 | 29% |
| NF37DV | SC | 2361 | Male | 4 | 33% |
| NF37DV | SC | 2362 | Male | 4 | 50% |
| NF37DV | SC | 2860 | Female | 4 | 29% |
| NF37DV | SC | 2861 | Female | 4 | 53% |
| NF37DV | SC | 2862 | Female | 4 | 18% |
| NF37DV | SC | 3360 | Male | 8 | 41% |
| NF37DV | SC | 3361 | Male | 8 | 42% |
| NF37DV | SC | 3362 | Male | 8 | 34% |
| NF37DV | SC | 3363 | Male | 8 | 51% |
| NF37DV | SC | 3364 | Male | 8 | 42% |
| NF37DV | SC | 3860 | Female | 8 | 21% |
| NF37DV | SC | 3861 | Female | 8 | 38% |
| NF37DV | SC | 3862 | Female | 8 | 38% |
| NF37DV | SC | 3863 | Female | 8 | 24% |
| NF37DV | SC | 3864 | Female | 8 | 20% |
| NF37DV | SC | 4360 | Male | 20 | 46% |
| NF37DV | SC | 4361 | Male | 20 | 35% |
| NF37DV | SC | 4362 | Male | 20 | 27% |
| NF37DV | SC | 4363 | Male | 20 | 53% |
| NF37DV | SC | 4364 | Male | 20 | 26% |
| NF37DV | SC | 4860 | Female | 20 | 50% |
| NF37DV | SC | 4861 | Female | 20 | 49% |
| NF37DV | SC | 4862 | Female | 20 | 14% |
| NF37DV | SC | 4863 | Female | 20 | 37% |
| NF37DV | SC | 4864 | Female | 20 | 45% |

Sources: Study TD36MM, DQ81GX and NF37DV.
ADA: anti-drug antibody; AUC₀₋ₜ: area under the concentration-time curve from the time of the last quantifiable sample; ID: identification; IV: intravenous(ly); SC: subcutaneous(ly).
^{a}Although not confirmed, it is believed that ADA development may have impacted the second IV dose in Study TD36MM, which impacted bioavailability calculations. Therefore, these values are not relevant when calculating the average bioavailability.

### Distribution

Volume of distribution at steady state (Vₛₛ) could only be calculated in a non-GLP study, Study QW56LH. Mean Vₛₛ was 37.6 mL/kg after the first IV dose and 47.55 mL/kg after the second IV dose. In subsequent non-GLP studies in monkeys, Vₛₛ could not be calculated because of insufficient characterization of the terminal phase after IV infusion.

In the GLP study, mean Vₛₛ was 91.9 and 71.1 mL/kg in males and females, respectively, after the first IV dose, with an overall mean Vₛₛ of 81.5 mL/kg across all 10 animals. On Day 15, the mean Vₛₛ was 107 mL/kg in males (n=2) and 108 mL/kg in the single female evaluated, for an overall mean Vₛₛ of 108 mL/kg across the 3 animals.

### Metabolism

Metabolism studies of DF hIL12-Fc si have not been conducted. Standard metabolism studies routinely conducted for small molecule drugs are not considered necessary or useful for biologics such as antibodies.

### Excretion

Excretion studies of DF hIL12-Fc si have not been conducted. Standard elimination studies routinely conducted for small molecule drugs are not considered necessary or useful for biologics such as DF hIL12-Fc si.

### Drug-Drug Interactions

No Drug-Drug interaction (DDI) studies have been performed to date. Therapeutic proteins such as cytokines or monoclonal antibodies that act as cytokine modulators are likely to exhibit interactions with small molecule drugs by influencing the expression and stability of specific cytokine p450 (CYP) enzymes and drug transporters (Huang S M, Zhao H, Lee J I, Reynolds K, Zhang L, Temple R, et al., Clin Pharmacol Ther. 2010; 87(4):497-503). Among cytokines, IL6 is known to downregulate CYP expression. Modeling shows IL-6 could reduce the intrinsic clearance of CYP3A4 by 28% at approximately 48 hours post-dose (Xu Y, Hijazi Y, Wolf A, Wu B, Sun Y N, Zhu M., CPT Pharmacometrics Syst Pharmacol. 2015; 4(9):507-15). DF hIL12-Fc si induced minimal and sporadic increases of IL6 in monkeys at tolerated doses. Given the de novo synthesis of CYP enzymes (t_{z} of 24 to 36 hours) and transient duration of the IL6 cytokine spike with DF hIL12-Fc si, the risk of DDIs is not considered significant.

Because there is no reason to believe that DF hIL12-Fc si, an IL12-Fc fusion protein, is metabolized by CYP enzymes, it is unlikely that a small molecule that inhibits or induces CYP enzymes could impact the PK of DF hIL12-Fc si. Based on these considerations, the PK DDI potential for DF hIL12-Fc si is considered to be low.

### Immunogenicity

In the single-dose toxicology study in monkeys, 7 of 12 treated animals were found to be positive for anti-drug antibody (ADA). Additionally, although ADA was not evaluated, variability in TK and anomalously high estimates of bioavailability in a 4-week repeat dose study in cynomolgus monkeys were considered consistent with the production of ADA to DF hIL12-Fc si. ADA impacted exposures of individual monkeys in both non-GLP and GLP studies, but suitable exposure was achieved for a long enough duration to confidently define the toxicology profile of DF hIL12-Fc si. ADA in monkeys is not predictive of immunogenicity in humans. For these reasons, serum titers of anti-DF hIL12-Fc si antibodies will be evaluated in clinical studies.

### Example 26: Treatment of Cancer Using DF hIL12-Fc Si

### Objectives

This clinical study is designed with the following phases: Phase 1, Phase 1b, and Phase 2.

The primary objective of Phase 1 is to assess the safety and tolerability of DF hIL12-Fc si as monotherapy, and to determine the maximum tolerated dose (MTD) of DF hIL12-Fc si in patients with advanced (unresectable, recurrent or metastatic) solid tumors.

The primary objective of Phase 1b is to assess the safety and tolerability of DF hIL12-Fc si in combination with pembrolizumab, and to determine the maximum tolerated dose (MTD) of DF hIL12-Fc si in combination with pembrolizumab in patients with advanced (unresectable, recurrent, or metastatic) solid tumors.

The primary objective of Phase 2 is to assess the Objective Response Rate (ORR) according to the Response Evaluation Criteria in Solid Tumors version 1.1 (RECIST 1.1) per an Independent Endpoint Review Committee (IERC), for all Efficacy Expansion Cohorts testing the clinical activity of DF hIL12-Fc si as a monotherapy or in combination.

The secondary objectives of Phase 1 and Phase 1b, with DF hIL12-Fc si as monotherapy and in combination with pembrolizumab, are to: Characterize the PK of DF hIL12-Fc si; evaluate immunogenicity of DF hIL12-Fc si, and to correlate its exposure and clinical activity; assess best overall response (BOR), as determined by the Investigator for DF hIL12-Fc si using RECIST 1.1; assess duration of response (DOR) of DF hIL12-Fc si, using RECIST 1.1; assess progression-free survival (PFS) for DF hIL12-Fc si, using RECIST 1.1; and assess overall survival (OS) time.

The secondary objectives of Phase 2, with DF hIL12-Fc si as monotherapy and in combination with pembrolizumab, are to: characterize the PK of DF hIL12-Fc si; assess duration of response (DOR) of DF hIL12-Fc si, per an IERC using RECIST 1.1; assess clinical benefit rate (CBR) of DF hIL12-Fc si using RECIST 1.1. CBR is defined as the percentage of patients with complete response (CR), partial response (PR), or stable disease (SD) as best response; assess the safety of DF hIL12-Fc si; evaluate the immunogenicity of DF hIL12-Fc si, and correlate with exposure and clinical activity; assess progression-free survival (PFS) for DF hIL12-Fc si, per an IERC using RECIST 1.1; and Assess overall survival (OS) time.

### Exploratory Objectives

The exploratory objectives, both with DF hIL12-Fc si as monotherapy and in combination with pembrolizumab, are to: evaluate changes from baseline in tumor and peripheral biomarkers, and the relationship to PK; assess the PK of pembrolizumab (Phase 1b and Cohort 2C only); evaluate the activity of DF hIL12-Fc si in the Efficacy Expansion Cohorts Part (Phase 2) per Investigator assessment (ORR, DOR, CBR, and BOR, by RECIST); and evaluate the association between tumor and peripheral biomarkers, and tumor response rate.

### Study Design Overview

This study is a Phase 1/2, open-label, dose-escalation study with a consecutive parallel-group efficacy expansion study, designed to determine the safety, tolerability, PK, pharmacodynamics, and preliminary anti-tumor activity of DF hIL12-Fc si as monotherapy and in combination with pembrolizumab. A schematic diagram of the study design is shown in FIG. 51A (for monotherapy) and 51B (for combination therapy with Pembrolizumab).

The study consists of 3 parts: Phase 1: Dose-escalation as a monotherapy using a 3+3 design, with Phase 1 Cohort Expansion; Phase 1b: Dose-escalation as a combination with pembrolizumab using a 3+3 design, with Phase 1b Cohort Expansion; and Phase 2: Efficacy Expansion using a group sequential design.

DF hIL12-Fc si is evaluated as a monotherapy in Efficacy Expansion cohorts in the following indications: Cohort 2A: Advanced (unresectable or metastatic) melanoma; and Cohort 2B: Advanced (unresectable or metastatic) renal cell carcinoma (RCC)

DF hIL12-Fc si is evaluated in combination with pembrolizumab in an Efficacy Expansion cohort in the following indication: Cohort C: Advanced (unresectable or metastatic) urothelial carcinoma

In each study phase, patients receive DF hIL12-Fc si on Day 1 every 3 weeks (Q3W). Patients receive DF hIL12-Fc si until confirmed progressive disease (PD), unacceptable toxicity (i.e., dose-limiting toxicity [DLT]), or any reason for withdrawal from the study or Investigational Medicinal Product (IMP) occurs.

Phase 1 Dose Escalation DF hIL12-Fc si Monotherapy

The Phase 1 Dose-escalation Phase of the study is designed to determine the dose-limiting toxicities (DLTs) and maximum tolerated dose (MTD) of DF hIL12-Fc si as monotherapy using a standard 3+3 design.

The decision to escalate to the next dose level (DL) is based on safety assessments after all patients of a cohort have had safety evaluations performed through Cycle 2, Day 1 (C2D1), unless due to DLT. In order to assess the safety of DF hIL12-Fc si, a Safety Monitoring Committee (SMC), responsible for dose-escalation decisions, is established.

After the safety of Dose Level “n” has been established, the SMC has the option to permit enrollment of up to 10 patients at that DL in the Phase 1 Expansion Cohort; no more than 30 patients can be enrolled by this process.

The MTD is defined as the highest DL at which ≤1 patient of 6 evaluable patients experiences a DLT.

Phase 1b: Dose-Escalation as a Combination with Pembrolizumab

The Phase 1b Dose-escalation Phase of the study is designed to determine the DLTs and MTD of DF hIL12-Fc si when given in combination with pembrolizumab, using a standard 3+3 design, as described for Phase 1.

Pembrolizumab is administered once every 3 weeks (on Day 1 of each cycle) per its U.S. package insert. The administration of pembrolizumab precedes that of DF hIL12-Fc si.

DF hIL12-Fc si dose levels tested in combination with pembrolizumab are the same as those tested as a monotherapy.

Phase 1b starts after any of the following criteria are met with DF hIL12-Fc si monotherapy: a Grade 2 drug-related toxicity occurs at any dose level occurring during the DLT observation period; a DLT occurs at a dose level not defined as the MTD; and dose-escalation is complete, with no MTD defined.

After one of these criteria are met, Phase 1b (DF hIL12-Fc si in combination with pembrolizumab) starts using a dose of DF hIL12-Fc si two dose levels below the one that met any of the above criteria, or if any of the criteria are met at DL1 or at DL2, the starting dose for combination is DL1, after the safety of DL1 has been established (defined by 3 patients treated at DL2 or 6 patients treated at DL1, with no more than one DLT observed at DL1).

After the safety of Dose Level “n” has been established, the SMC has the option to permit enrollment of up to 10 patients at that dose level in the Phase 1b Expansion Cohort; no more than 30 patients can be enrolled by this process.

### Phase 2 Efficacy Expansion

The following tumor types are enrolled at the recommended phase 2 dose (RP2D):

As a monotherapy: Cohort 2A: Advanced (unresectable or metastatic) melanoma; and Cohort 2B: Advanced (unresectable or metastatic) renal cell carcinoma.

In combination with pembrolizumab: Cohort 2C: Advanced (unresectable or metastatic) urothelial carcinoma.

### Inclusion and Exclusion Criteria

Male or female patients aged ≥18 years with an Eastern Cooperative Oncology Group (ECOG) performance status of 0 or 1 at study entry and an estimated life expectancy of at least 3 months are enrolled.

Key inclusion criteria in each study phase/cohort are as follows:

Dose-Escalation cohorts in Phase 1/1b: Clinical or radiological evidence of disease

Dose Expansion Cohorts in Phase 1/1b: Has one of the following tumor types: melanoma, non-small cell lung cancer (NSCLC), small-cell lung cancer (SCLC), head and neck squamous cell carcinoma (HNSCC), classical Hodgkin lymphoma, primary mediastinal large B-Cell lymphoma, urothelial carcinoma, micro-satellite instability high cancer, gastric cancer, oesophageal cancer, cervical cancer, hepatocellular cancer, Merkel cell carcinoma, renal cell carcinoma, endometrial cancer, cutaneous T cell lymphoma, or triple negative breast cancer; has measurable disease, as determined by the Investigator using the Response Evaluation Criteria for Solid Tumors (RECIST), version 1.1;

Cohort 2A

Patients with advanced melanoma who: received treatment with an anti-programmed cell death protein 1 (PD-1) antibody for at least 6 weeks; have a confirmation of PD at least 4 weeks after the initial diagnosis of PD while receiving an anti PD-1 is made. Confirmation of PD can be based on radiological or clinical observations; must have received a BRAF inhibitor if the tumor carries a BRAF activating mutation and have progressed after the last line of treatment.

Cohort 2B

Patients with advanced RCC who: have any clear cell histology component; received treatment with an anti PD-1/PD-L1 antibody and an anti-vascular endothelial growth factor therapy as a monotherapy or in combination; received ≤3 prior lines of therapy

Cohort 2C

Patients with advanced urothelial carcinoma who: have histologically or cytologically documented locally advanced or metastatic transitional cell carcinoma of the urothelium (including renal pelvis, ureters, urinary urothelial, urethra); have received one (and no more than one) platinum-containing regimen (e.g., platinum plus another agent such as gemcitabine, methotrexate, vinblastine, doxorubicin, etc.) for inoperable locally advanced or metastatic urothelial carcinoma with radiographic progression or with recurrence within 6 months after the last administration of a platinum-containing regimen as an adjuvant, which would be considered failure of a first-line, platinum-containing regimen; have received no more than 2 lines of therapy (including the platinum-containing regimen) for the treatment of the metastatic disease; have not received treatment with a checkpoint inhibitor (CPI) (i.e., anti-PD-1 or anti-PD-L1 as a monotherapy or in combination with a platinum-based chemotherapy.

### Dose Mode of Administration Dosing Schedule

DF hIL12-Fc si is administered as a subcutaneous (SC) injection Q3W (i.e., on Day 1 of each cycle). Patients receive the drug SC in a volume of not more than 1 mL in a maximum of 2 injection sites. The second administration is completed within 10 minutes after the completion of the first administration, if applicable.

In Phase 1/1b, patients are hospitalized for the night following the first administration of DF hIL12-Fc si.

The DF hIL12-Fc si DLs (μg/kg) are as follows in Table 73.

TABLE 73
DF hIL12-Fc si DLs (μg/kg)

| | DL1 | DL2 | DL3 | DL4 | DL5 | DL6 | DL7 | DL8 | DL9 | DL10 |
|---|---|---|---|---|---|---|---|---|---|---|
| Dose of DF | 0.05 | 0.10 | 0.20 | 0.40 | 0.60 | 0.80 | 1.00 | 1.20 | 1.40 | 1.75 |
| hIL12-Fc si | | | | | | | | | | |
| (μ/kg) | | | | | | | | | | |
| Equivalent | 0.021 | 0.053 | 0.105 | 0.211 | 0.316 | 0.421 | 0.526 | 0.632 | 0.737 | 0.921 |
| IL12 | | | | | | | | | | |
| (μg/kg) | | | | | | | | | | |

The dose of DF hIL12-Fc si is calculated based on the weight of the patient at baseline. The patient's calculated dose is only recalculated if the patient's weight changes by 10% or more since the time of their last dose calculation.

### Exploratory Biomarkers

### Peripheral Biomarkers

Peripheral biomarkers are assessed in the periphery in all patients, including: cellular parameters: peripheral blood mononuclear cell (PBMCs) for immunophenotyping (IPT) by flow cytometry; soluble factors: Cytokines and chemokines in serum samples; ex vivo IL12 response assay: PBMCs for ex vivo stimulation followed by analysis of IFNγ production; circulating tumor (ct) deoxyribonucleic acid (DNA).

IPT assessments are performed on PBMCs derived from whole blood samples are collected 2 hours prior to administration of DF hIL12-Fc si C1 through C3 and at each of the following study visits: C1D3, C1D8, C2D8, and C3D3.

Soluble factors are determined in serum samples collected within 2 hours prior to DF hIL12-Fc si administration on D1 of each treatment cycle, and on C1D2, C1D3, C1D5, C1D8, C1D15, C2D3, C3D3, and C4D3, and at the EOT and SFU visits.

In order to complete all the assessments on tumor materials, blood (e.g., whole blood, plasma, and serum samples), is collected from patients.

### Biomarkers Derived From Tumor Tissue

Tissue derived biomarkers are evaluated on the pre-treatment and on treatment biopsies in patients participating in the Dose-escalation phase (optional biopsies), the Phase 1/1b Expansion Cohorts part (mandatory biopsies), and the Phase 2 Efficacy Expansion Cohorts phase (mandatory biopsies).

A panel of putative markers including molecular, soluble and cellular markers is analyzed at baseline from archived tumor tissue (or fresh tumor biopsy, if available), whole blood, and serum samples to investigate a possible correlation between clinical efficacy and analyzed markers.

For patients enrolled in the Dose-escalation Phase, the level of PD-L1 expression is determined using a commercially available kit (Dako PD-L1 IHC 22C3 pharmDx) and analysis of CD3 positivity (T cell infiltration) is determined by immunohistochemistry (IHC).

For patients enrolled in the Phase 1/1b Expansion Cohorts and the Efficacy Expansion Cohorts, fresh mandatory tumor biopsies is performed at Screening (i.e., within 30 days before first study drug dose) and at pre-specified time points during the treatment period.

Other biomarkers that are assessed include: frequency and localization of tumor-infiltrated leukocytes (eg, CD8, CD4 T-cells, Treg, NK cells, macrophage [M1/2 profile] by IHC or IF), gene expression profile, and pharmacogenomics (PGx).

### Reference Therapy: Dose Mode of Administration Dosing Schedule

In Phase 1b and Cohort 2C, pembrolizumab is administered at a dose of 200 mg, once every 3 weeks via intravenous (IV) infusion, in accordance with the U.S. package insert. The administration of pembrolizumab precedes that of DF hIL12-Fc si. DF hIL12-Fc si is administered within 1 hour after the completion of the administration of pembrolizumab.

### Planned Treatment Duration Per Patient

Patients receive study treatment until development of progressive disease (PD) or unacceptable toxicity, or any criterion for withdrawal from the study or DF hIL12-Fc si occurs.

Any patients who have experienced a confirmed complete response (CR) are treated for at least 12 months after confirmation, unless a criterion for discontinuation is met, at the discretion of the Investigator. If the Investigator believes that such a patient may benefit from treatment beyond 12 months, it may be permissible to continue the treatment after discussion with the Sponsor Medical Monitor. The maximum treatment duration is 24 months.

### Statistical Methods (Includes Sample Size Calculation)

The number of the evaluable patients for this study is derived from the dose-escalation “3+3” design and the expansion cohort sizes. The final sample size may vary depending on the total number of DLs that are evaluated, patient replacement for DLT evaluation, if applicable, and expansion from 3 to 6 patients if a DLT is observed.

In the event that rapid recruitment in the expansion phase impacts supply of IMP, the screening of new patients for any cohort may be temporarily paused with 24-hours notice to Investigators.

The final sample size may vary depending on the total number of DLs that are evaluated, patient replacement for DLT evaluation, if applicable, and expansion from 3 to 6 patients if a DLT is observed.

### Efficacy Expansion as a Monotherapy (Cohorts 2A and 2B)

The primary endpoint for this phase is the ORR. For each of these cohorts, the null hypothesis is that the objective response rate (ORR) does not exceed 5% (H0: ORR<5%) and the alternative hypothesis is that the ORR is greater than 10% (H1: ORR ≥5%).

The target ORR of DF hIL12-Fc si as a monotherapy is 20%. It is expected to enroll 40 patients for each of these cohorts (i.e., approximately 80 patients in total).

Using a group sequential design, with 40 patients in each of the indication cohorts, the efficacy cohort provides ˜90% study power to detect a 15% difference at a 1-sided overall type I error rate of 0.025, assuming the target ORR of 20% for DF hIL12-Fc si.

For each of Cohorts 2A and 2B, a futility interim analysis, with Lan-DeMets O'Brien Fleming boundary, is planned at 50% information fraction (i.e., at ˜20 patients).

The enrollment may be stopped for futility once 20 patients have completed 3 months follow-up or have withdrawn from the study, if none of the enrolled patients have achieved an unconfirmed BOR of PR or CR according to RECIST 1.1. At the end of the study, a cohort is declared successful if at least 5 patients achieve a confirmed BOR of PR or CR according to RECIST 1.1.

Efficacy Expansion in Combination with Pembrolizumab (Cohort 2C)

The Phase 2 portion for efficacy expansion in combination with pembrolizumab determines the clinical activity of DF hIL12-Fc si in combination in patients with UBC who have progressed after one line of platinum-based chemotherapy.

The primary endpoint is the ORR. The study enrolls 40 patients so that the observation of 15 responses (CR or PR) out of the 40 patients enroll will lead to a 95% CI (0.2317; 0.5419) that excludes the value of the percentage of responses reported for pembrolizumab in a similar population, that was enrolled in KEYNOTE-045. In that study, the ORR was 21.7% (Bellmunt J, de Wit R, Vaughn D J, Fradet Y, Lee J L, Fong L, et al., N Engl J Med. 2017; 376(11):1015-1026).

A minimum 4-week metric is used to qualify SD and to confirm CR, PR or PD. DOR is defined from the time between the first observation of a CR or PR and disease progression. PFS, is defined according to RECIST 1.1 and defined from first administration of study treatment until first observation of progressive disease or death, whichever comes first. OS is defined as the time from first administration of study treatment to death.

Safety analyses are summarized by descriptive statistical presentation, by cohort and/or across cohorts.

### Example 27: Treatment of Cancer Using DF hIL12-Fc Si in a Single Dose

The primary objective of this study is to assess the safety and tolerability of DF hIL12-Fc si as a monotherapy when administered in a single dose, and to determine the maximum tolerated dose (MTD) of DF hIL12-Fc si in patients with advanced (unresectable, recurrent or metastatic) solid tumors. DF hIL12-Fc si is administered as a subcutaneous (SC) injection in a single dose. Patients receive the drug SC in a volume of not more than 1 mL in a maximum of 2 injection sites. The second administration is completed within 10 minutes after the completion of the first administration, if applicable. The patients receive only the single dose of DF hIL12-Fc si.

### Example 28: Treatment of Cancer Using DF hIL12-Fc Si

This is a Phase 1/2, open-label, dose-escalation study with a consecutive parallel-group efficacy expansion study, designed to determine the safety, toleratbility, PK, pharmacodynamics, and preliminary anti-tumor activity of DF-hIL-12-Fc si as monotherapy and in combination with pembrolizumab.

TABLE 74
Phase 1/2, Open-Label, Dose-Escalation Study

| Condition or Disease | Intervention/Treatment | Phase |
|---|---|---|
| Solid Tumor; Melanoma; | Biological/Vaccine: | Phase 1/ |
| renal cell carcinoma; | DF-hIL-12-Fc si; | Phase 2 |
| urothelial carcinoma | Biological/Vaccine: | |
| | Pembrolizumab | |

The study consists of 3 parts: Phase 1: Dose-escalation as a monotherapy using a 3+3 design, with Phase 1 Cohort Expansion; Phase 1b: Dose-escalating as a combination with pembrolizumab using a 3+3 design, with Phase 1b cohort Expansion; Phase 2: Efficacy Expansion using a group sequential design.

In phase 2, DF-hIL-12-Fc si is evaluated as a monotherapy in the following indications: Cohort 2A: Advanced (unresectable or metastatic) melanoma; Cohort 2B: Advanced (unresectable or metastatic) renal cell carcinoma (RCC).

In phase 2, DF-hIL-12-Fc si is evaluated in combination with pembrolizumab in the following indication: Cohort C: Advanced (unresectable or metstatic) urothelial carcinoma.

In each study phase, patent is receive DF-hIL-12-Fc si on Day 1 every 3 weeks (Q3W). Patients receive DF-hIL-12-Fc si until confirmed progressive disease (PD), unacceptable toxicity (i.e., dose-limiting toxicity [DSL]), or any reason for withdrawal from the study or Investigational Medicinal Product (IMP) occurs.

The arms and interventations are presented in Table 75 below.

TABLE 75
Arms and Interventions

| Arm | Intervention/Treatment |
|---|---|
| Experimental DF-hIL-12-Fc si | Biological/Vaccine DF-hIL-12-Fc si; |
| Monotherapy Dose Escalation; | DF-hIL-12-Fc si is a monovalent human |
| 3 + 3 dose escalation of subcutaneous DF- | interleukin-12 (IL-12) constant fragment |
| hIL-12-Fc si as monotherapy in patients with | (Fc) fusion protein that binds to the IL-12 |
| solid tumors. | receptor. |
| Experimental: DF-hIL-12-Fc si | Biological/Vaccine: DF-hIL-12-Fc si; |
| Monotherapy Expansion (Melanoma); | DF-hIL-12-Fc si is a monovalent human |
| Dose expansion of up to 40 | interleukin 12 (IL-12)-constant fragment |
| patients with melanoma receiving | (Fc) fusion protein that binds to the IL-12 |
| subcutaneous DF-hIL-12-Fc si as | receptor |
| monotherapy. | |
| Experimental: DF-hIL-12-Fc si | Biological/Vaccine: DF-hIL-12-Fc si; |
| Monotherapy Expansion (Renal Cell); | DF-hIL-12-Fc si is a monovalent human |
| Dose expansion of up to 40 patients with | interleukin 12 (IL-12)-constant fragment |
| renal cell carcinoma receiving subcutaneous | (Fc) fusion protein that binds to the IL-12 |
| DF-hIL-12-Fc si as monotherapy | receptor |
| Experimental: DF-hIL-12-Fc si In | Biological/Vaccine: DF-hIL-12-Fc si; |
| Combination with Keytruda Escalation; 3 + 3 | DF-hIL-12-Fc si is a monovalent human |
| dose escalation of subcutaneous DF-hIL-12- | interleukin-12 (IL-12) constant fragment |
| Fc si in combination with intravenous | (Fc) fusion protein that binds to the IL-12 |
| Keytruda. | receptor; |
| | Biological/Vaccine: Pembrolizumab; |
| | Pembrolizumab is a potent and highly |
| | selective humanized mAb of the |
| | Immunoglobulin (IgG4)/kappa isotype |
| | designed to directly block the interaction |
| | between PD-1 and its ligands, PD-L1 and |
| | PD-L2; Other names: Keytruda |
| Experimental: DF-hIL-12-Fc si in | Biological/Vaccine: DF-hIL-12-Fc si; |
| Combination with Keytruda | DF-hIL-12-Fc si is a monovalent human |
| Expansion (Urothelial); | interleukin-12 (IL-12)-constant fragment |
| Dose expansion of up to 40 patients with | (Fc) fusion protein that binds to the IL-12 |
| urothelial carcinoma receiving subcutaneous | receptor; |
| DF-hIL-12-Fc si in combination with | Biological/Vaccine: Pembrolizumab; |
| intravenous Keytruda. | Pembrolizumab is a potent and highly |
| | selective humanized mAb of the |
| | Immunoglobulin (IgG4)/kappa isotype |
| | designed to directly block the interaction |
| | between PD-1 and its ligands, PD-L1 and |
| | PD-L2; Other names: Keytruda |

The primary outcome measures include: 1. Assessment of the number of dose limiting toxicities experienced on study with monotherapy DF-hIL-12-Fc si as defined per criteria in the study protocol [Time Frame: First 3 weeks on treatment for each subject]; To assess the number of adverse events experienced during treatment with monotherapy DF-hIL-12-Fc si that meet dose limiting toxicity criteria per the study protocol; 2. Assessment of the number of dose limiting toxicities experienced on study with combination therapy of DF-hIL-12-Fc si and pembrolizumab as defined per criteria in the study protocol [Time Frame: First 3 weeks on treatment for each subject in the combination therapy cohort]; To assess the number of adverse events experienced during treatment with combination therapy of DF-hIL-12-Fc si and pembrolizumab that meet dose limiting toxicity; criteria per the study protocol; 3. Assess overall response rate [Time Frame: Through 90 days after completion of the study, an average of 1 year]; To assess the Overall Response Rate (ORR) per RECIST version 1.1 criteria of patients in the Phase 2 expansion cohorts.

The secondary outcome measures include: 1. Assess number of treatment emergent adverse events throughout study [Time Frame: Until 30 days after the last treatment of the last subject enrolled in the Phase 2 portion of the study]; Characterize the safety profile of DF-hIL-12-Fc si by assessing the number of adverse events occurring while on treatment with DF-hIL-12-Fc si; 2. Determine serum concentrations of DF-hIL-12-Fc si at various timepoints [Time Frame: From start of treatment up through 28 days after last treatment]; Concentration vs time of DF-hIL-12-Fc si will be measured using blood samples taken a various time points on study; 3. Assess Duration of Response [Time Frame: From time of initiation of therapy until the date of first documented tumor progression, assessed up to 24 months]; To assess duration of response using RECIST 1.1 criteria; 4. Assess Best Overall Response [Time Frame: Through 90 days after completion of the study, an average of 1 year]; To assess best overall response using RECIST 1.1 criteria; 5. Assess Overall Survival [Time Frame: Time from enrollment in the study until death, measured up to 2 years after last treatment on study]; To assess overall survival following treatment; 6. Assess Overall Response Rate [Time Frame: lime from enrollment in the study until up to 2 years after last treatment on study]; To assess overall response rate according to Investigator judgment.

### Eligibility Criteria

The inclusion criteria (general phase 1) are: 1. Signed written informed consent; 2. Male or female patients aged ≥18 years; 3. Histologically or cytologically proven locally advanced or metastatic solid tumors, for which no standard therapy exists, or standard therapy has failed; 4. ECOG performance status of 0 or 1 at study entry and an estimated life expectancy of at least 3 months; 5. Clinical or radiological evidence of disease; 6. Archived tumor biopsy available, not older than 6 months, at least 8 slides available; or optional fresh biopsy obtained within the Screening window; 7. Adequate hematological function defined by white blood cell (WBC) count ≥3×10⁹/L with absolute neutrophil count (ANC) ≥1.5×10⁹/L, lymphocyte count ≥0.5×10⁹/L, platelet count ≥75×10⁹/L, and hemoglobin ≥9 g/dL (may have been transfused); 8. Adequate hepatic function defined by a total bilirubin level ≤1.5× the upper limit of normal (ULN), an AST level ≤2.5×ULN, and an ALT level ≤2.5×ULN or, for patients with documented metastatic disease to the liver, AST and ALT levels ≤5×ULN; 9. Adequate renal function defined by an estimated creatinine clearance 50 ml/min according to the Cockcroft-Gault formula; 10. Experienced resolution of toxic effect(s) of the most recent prior anti-cancer therapy to ≤Grade 1 (except alopecia) per NCI CTCAE v5.0 If a patient underwent major surgery or radiation therapy of >30 Gray, the patient must have recovered from the toxicity and/or complications from the intervention (Patients with ≤Grade 2 neuropathy or ≤Grade 2 alopecia are an exception to this criterion and may qualify for the study); 11. Effective contraception for women of child bearing potential (WOCBP) patients as defined by World Health Organization (WHO) guidelines for 1 “highly effective” method or 2 “effective” methods; 12. Eligible to receive pembrolizumab per its approved label. (Combination cohorts only).

Additional Phase 1 Monotherpahy Expansion Inclusion Criteria are: 1. Has one of the following tumor types: melanoma, non-small cell lung cancer (NSCLC), small cell lung cancer (SCLC), head and neck squamous cell carcinoma (HNSCC), classical Hodgkin lymphoma, primary mediastinal large B-Cell lymphoma, urothelial carcinoma, micro satellite instability high cancer, gastric cancer, oesophageal cancer, cervical cancer, hepatocellular cancer, Merkel cell carcinoma, renal cell carcinoma, endometrial cancer, cutaneous T cell lymphoma, or triple negative breast cancer; 2. Measurable disease, as determined by the Investigator using RECIST, version 1.1; 3. Agrees to undergo a pre-treatment biopsy and another biopsy while on treatment; 4. Has a clinical/radiological presentation of their disease consistent with the execution of a pre-treatment biopsy and another biopsy while on treatment.

Additional Phase 1b Combination Therapy Expansion Cohort Criteria are: 1. Measurable disease, as determined by the Investigator using RECIST, version 1.1; 2. Agrees to undergo a pre-treatment biopsy and another biopsy while on treatment; 3. Has a clinical/radiological presentation of their disease consistent with the execution of a pre-treatment biopsy and another biopsy while on treatment.

Inclusion criteria (General Phase 2) are: 1. Signed written informed consent; 2. Male or female patients aged ≥18 years; 3. ECOG performance status of 0 or 1 at study entry and an estimated life expectancy of at least 3 months; 4. Measurable disease, as determined by the Investigator using RECIST, version 1.1; 5. Agrees to undergo a pre-treatment biopsy and another biopsy while on treatment; 6. Adequate hematological function defined by WBC count ≥3×10⁹/L with ANC ≥1.5×10⁹/L, lymphocyte count ≥0.5×10⁹/L, platelet count ≥75×10⁹/L, and hemoglobin ≥9 g/dL (may have been transfused); 7. Adequate hepatic function defined by a total bilirubin level ≤1.5×ULN, an AST level ≤2.5×ULN, and an ALT level ≤2.5×ULN or, for patients with documented metastatic disease to the liver, AST and ALT levels ≤5×ULN; 8. Adequate renal function defined by an estimated creatinine clearance >50 ml/min according to the Cockcroft-Gault formula; 9. Experienced resolution of toxic effect(s) of the most recent prior anti-cancer therapy to Grade ≤1 (except alopecia) per NCI CTCAE v5.0 If a patient underwent major surgery or radiation therapy of >30 Gray, the patient must have recovered from the toxicity and/or complications from the intervention (Patients with ≤Grade 2 neuropathy or ≤Grade 2 alopecia are an exception to this criterion and may qualify for the study); 10. Has a clinical/radiological presentation of their disease consistent with the execution of a pre-treatment biopsy and another biopsy while on treatment; 11. Effective contraception for WOCBP patients as defined by WHO guidelines for 1 “highly effective” method or 2 “effective” methods.

Additional Phase 2 Inclusion Criteria (Melanoma only) are: 1. Received treatment with an anti PD-1 antibody for at least 6 weeks; 2. Have a confirmation of PD at least 4 weeks after the initial diagnosis of PD while receiving an anti PD-1 is made. Confirmation of PD can be based on radiological or clinical observations; 3. Must have received a BRAF inhibitor if the tumor carries a BRAF activating mutation and have progressed after the last line of treatment.

Additional Phase 2 Inclusion Criteria (Renal Cell only) are: 1. Any clear cell histology component; 2. Received treatment with an anti PD-1/PD-L 1 antibody or an anti-vascular endothelial growth factor therapy as monotherapy or in combination; 3. Received ≤3 prior lines of therapy.

Additional Phase 2 Inclusion Criteria (Urothelial Carcinoma only) are: 1. Histologically or cytologically documented locally advanced or metastatic transitional cell carcinoma of the urothelium (including renal pelvis, ureters, urinary urothelial, urethra); 2. Must have received one (and no more than one) platinum-containing regimen (e.g., platinum plus another agent such as gemcitabine, methotrexate, vinblastine, doxorubicin) for inoperable locally advanced or metastatic urothelial carcinoma with radiographic progression or with recurrence within 6 months after the last administration of a platinum-containing regimen as an adjuvant, which would be considered failure of a first-line, platinum-containing regimen; 3. Have received no more than 2 lines of therapy (including the platinum-containing regimen) for the treatment of the metastatic disease; 4. Must NOT have received treatment with a CPI (i.e., anti-PD-1 or anti-PD-L1 as a monotherapy or in combination with a platinum-based chemotherapy.

The Exclusion Criteria are: 1. Concurrent treatment with a non-permitted drug; 2. Prior treatment with rhIL2 or any recombinant long acting drug containing an IL2 moiety; 3. Concurrent anticancer treatment (eg, cytoreductive therapy, radiotherapy [with the exception of palliative bone directed radiotherapy], immune therapy, or cytokine therapy except for erythropoietin), major surgery (excluding prior diagnostic biopsy), concurrent systemic therapy with steroids or other immunosuppressive agents, or use of any investigational drug within 28 days before the start of study treatment. Short-term administration of systemic steroids (ie, for allergic reactions or the management of irAEs) is allowed; Note: Patients receiving bisphosphonates are eligible provided treatment was initiated at least 14 days before the first dose of DF-hIL-12-Fc si; 4. Previous malignant disease other than the target malignancy to be investigated in this study within the last 3 years, with the exception of basal or squamous cell carcinoma of the skin, localized prostate cancer or cervical carcinoma in situ; 5. Rapidly progressive disease; 6. Any Grade 2 and higher neurological or pulmonary toxicity during a treatment with an anti-PD-1 or PD-L1 agent administered as a monotherapy; 7. Active or history of central nervous system (CNS) metastases. Melanoma patients with brain metastasis(ses) are eligible if they have been stable for 4 weeks after treatment; 8. Receipt of any organ transplantation including autologous or allogeneic stem-cell transplantation; 9. Significant acute or chronic infections (including historic positive test for human immunodeficiency virus [HIV], or active or latent hepatitis B or active hepatitis C tested during the Screening window); 10. Preexisting autoimmune disease (except for patients with vitiligo) needing treatment with systemic immunosuppressive agents for more than 28 days within the last 3 years or clinically relevant immunodeficiencies (eg, dys-gammaglobulinemia or congenital immunodeficiencies), or fever within 7 days of Day 1; 11. Known severe hypersensitivity reactions to monoclonal antibodies (mAbs) (≥Grade 3 NCI CTCAE v5.0), any history of anaphylaxis, or uncontrolled asthma (ie, 3 or more features of partly controlled asthma); 12. Persisting toxicity related to prior therapy ≥Grade 2 NCI CTCAE v5.0, however alopecia and sensory neuropathy ≤Grade 2 is acceptable; 13. Pregnancy or lactation in females during the study; 14. Known alcohol or drug abuse; 15. Serious cardiac illness or medical conditions including but not limited to: a. History of New York Heart Association class III or IV heart failure or systolic dysfunction (left ventricular ejection fraction [LVEF]<55%); b. High-risk uncontrolled arrhythmias ie, tachycardia with a heart rate >100/min at rest; c. Significant ventricular arrhythmia (ventricular tachycardia) or higher-grade atrioventricular (AV)-block (second degree AV-block Type 2 [Mobitz 2) or third-degree AV-block); d. Angina pectoris requiring anti-anginal medication; e. Clinically significant valvular heart disease; f Evidence of transmural infarction on ECG; g. Poorly controlled hypertension (defined by: systolic >180 mn Hg or diastolic >100 mmHg); h. Clinically relevant uncontrolled cardiac risk factors, clinically relevant pulmonary disease or any clinically relevant medical condition in the opinion of the Investigator that may limit participation in this study; 16. All other significant diseases (e.g., inflammatory bowel disease), which, in the opinion of the Investigator, might impair the patient's ability to participate; 17. Any psychiatric condition that would prohibit the understanding or rendering of informed consent; 18. Legal incapacity or limited legal capacity; 19. Incapable of giving signed informed consent, which includes compliance with the requirements and restrictions listed in the informed consent form (ICF) and in this protocol.

### Example 29: Treatment of Cancer Using DF hIL12-Fc Si

### Objectives

This clinical study is designed with the following phases: Phase 1, Phase 1b, and Phase 2.

The primary objective of Phase 1 is to assess the safety and tolerability of DF hIL12-Fc si (also referred to as DF6002) as monotherapy, and to determine the maximum tolerated dose (MTD) of DF hIL12-Fc si in patients with advanced (unresectable, recurrent or metastatic) solid tumors.

The primary objective of Phase 1b is to assess the safety and tolerability of DF hIL12-Fc si in combination with Nivolumab, and to determine the maximum tolerated dose (MTD) of DF hIL12-Fc si in combination with Nivolumab in patients with advanced (unresectable, recurrent, or metastatic) solid tumors.

The primary objective of Phase 2 is to assess the Objective Response Rate (ORR) according to the Response Evaluation Criteria in Solid Tumors version 1.1 (RECIST 1.1) per an Independent Endpoint Review Committee (IERC), for all Efficacy Expansion Cohorts testing the clinical activity of DF hIL12-Fc si as a monotherapy or in combination.

The secondary objectives of Phase 1 and Phase 1b, with DF hIL12-Fc si as monotherapy and in combination with Nivolumab, are to: characterize the PK of DF hIL12-Fc si; evaluate immunogenicity of DF hIL12-Fc si, and to correlate its exposure and clinical activity; assess best overall response (BOR), as determined by the Investigator for DF hIL12-Fc si using RECIST 1.1; assess best overall response (BOR), as determined by the Investigator for DF hIL12-Fe si using RECIST 1.1; assess duration of response (DOR) of DF hIL12-Fc si, using RECIST 1.1; assess progression-free survival (PFS) for DF hIL12-Fc si, using RECIST 1.1; assess overall survival (OS) time.

The secondary objectives of Phase 2, with DF hIL12-Fc si as monotherapy and in combination with Nivolumab, are to: characterize the PK of DF hIL12-Fc si; assess duration of response (DOR) of DF hIL2-Fc si, per an IERC using RECIST 1.1; assess clinical benefit rate (CBR) of DF hIL12-Fc si using RECIST 1.1. CBR is defined as the percentage of patients with complete response (CR), partial response (PR), or stable disease (SD) as best response; assess the safety of DF hIL12-Fc si; evaluate the immunogenicity of DF hIL2-Fc si, and correlate with exposure and clinical activity; assess progression-free survival (PFS) for DF hIL2-Fc si, per an IERC using RECIST 1.1; and assess overall survival (OS) time.

The arms and interventions are presented in Table 76 below:

TABLE 76
Arms and Interventions

| Arm | Intervention/Treatment |
|---|---|
| Experimental: DF6002 Monotherapy Dose | Biological: DF6002; DF6002 is a |
| Escalation; 3 + 3 dose escalation of | monovalent human interleukin-12 (IL12)- |
| subcutaneous DF6002 as monotherapy in | constant fragment (Fc) fusion protein that |
| patients with solid tumors. | binds to the IL12 receptor. |
| Experimental: DF6002 Monotherapy | Biological: DF6002; DF6002 is a |
| Expansion (Melanoma); Dose expansion of | monovalent human interleukin-12 (IL12)- |
| up to 40 patients with melanoma receiving | constant fragment (Fc) fusion protein that |
| subcutaneous DF6002 as monotherapy. | binds to the IL12 receptor. |
| Experimental: DF6002 Monotherapy | Biological: DF6002; DF6002 is a |
| Expansion (NSCLC); Dose expansion of up | monovalent human interleukin-12 (IL12)- |
| to 40 patients with non-small cell lung cancer | constant fragment (Fc) fusion protein that |
| receiving subcutaneous DF6002 as | binds to the IL12 receptor |
| monotherapy. | |
| Experimental: DF6002 In Combination with | Biological: DF6002 |
| Opdivo Escalation; 3 + 3 dose escalation of | DF6002 is a monovalent human interleukin- |
| subcutaneous DF6002 in combination with | 12 (IL12)-constant fragment (Fc) fusion |
| intravenous Opdivo. | protein that binds to the IL12 receptor. |
| | Biological: Nivolumab |
| | Nivolumab is a human immunoglobulin G4 |
| | (IgG4) monoclonal antibody, which binds to |
| | the programmed death-1 receptor (PD-1). |
| | Other Name: Opdivo |
| Experimental: DF6002 in Combination with | Biological: DF6002 |
| Opdivo Expansion (Melanoma) | DF6002 is a monovalent human interleukin- |
| Dose expansion of up to 40 patients with | 12 (IL12)-constant fragment (Fc) fusion |
| melanoma receiving subcutaneous DF6002 | protein that binds to the IL12 receptor. |
| in combination with intravenous Opdivo. | Biological: Nivolumab |
| | Nivolumab is a human immunoglobulin G4 |
| | (IgG4) monoclonal antibody, which binds to |
| | the programmed death-1 receptor (PD-1). |
| | Other Name: Opdivo |
| Experimental: DF6002 in Combination with | Biological: DF6002 |
| Opdivo Expansion (NSCLC) | DF6002 is a monovalent human interleukin- |
| Dose expansion of up to 40 patients with | 12 (IL12)-constant fragment (Fc) fusion |
| non-small cell lung cancer receiving | protein that binds to the IL12 receptor. |
| subcutaneous DF6002 in combination with | Biological: Nivolumab |
| intravenous Opdivo. | Nivolumab is a human immunoglobulin G4 |
| | (IgG4) monoclonal antibody, which binds to |
| | the programmed death-1 receptor (PD-1). |
| | Other Name: Opdivo |

The primary outcome measures include:

1. Assessment of the number of dose limiting toxicities experienced on study with monotherapy DF6002 as defined per criteria in the study protocol [Time Frame: First 3 weeks on treatment for each subject.] To assess the number of adverse events experienced during treatment with monotherapy DF6002 that meet dose limiting toxicity criteria per the study protocol;

2. Assessment of the number of dose limiting toxicities experienced on study with combination therapy of DF6002 and nivolumab as defined per criteria in the study protocol [Time Frame: First 3 weeks on treatment for each subject in the combination therapy cohort.] To assess the number of adverse events experienced during treatment with combination therapy of DF6002 and nivolumab that meet dose limiting toxicity criteria per the study protocol; and

3. Assess overall response rate [Time Frame: Through 90 days after completion of the study, an average of 1 year.] To assess the Overall Response Rate (ORR) per RECIST version 1.1 criteria of patients in the Phase 2 expansion cohorts. The secondary outcome measures include: 1. Assess number of treatment emergent adverse events throughout study [Time Frame: Until 30 days after the last treatment of the last subject enrolled in the Phase 2 portion of the study.] Characterize the safety profile of DF6002 by assessing the number of adverse events occurring while on treatment with DF6002; 2. Determine serum concentrations of DF6002 at various timepoints [Time Frame: From start of treatment up through 28 days after last treatment] Concentration vs time of DF6002 will be measured using blood samples taken a various time points on study;

4. Assess Duration of Response [Time Frame: From time of initiation of therapy until the date of first documented tumor progression, assessed up to 24 months] To assess duration of response using RECIST 1.1 criteria;

5. Assess Best Overall Response [Time Frame: Through 90 days after completion of the study, an average of 1 year] To assess best overall response using RECIST 1.1 criteria;

6. Assess Overall Survival [Time Frame: Time from enrollment in the study until death, measured up to 2 years after last treatment on study] To assess overall survival following treatment; and

7. Assess Overall Response Rate [Time Frame: Time from enrollment in the study until up to 2 years after last treatment on study] To assess overall response rate according to Investigator judgment.

The inclusion criteria (General Phase 1 and Phase 1b) include: 1. Male or female patients aged ≥18 years; 2. Histologically or cytologically proven locally advanced or metastatic solid tumors, for which no standard therapy exists or standard therapy has failed among the following tumor types: melanoma, non-small cell lung cancer, small cell lung cancer, head and neck squamous cell, urothelial, gastric, esophageal, cervical, hepatocellular, merkel cell, cutaneous squamous cell carcinoma, renal cell, endometrial, triple-negative breast, ovarian, and prostate; 3. ECOG performance status of 0 or 1; 4. Clinical or radiological evidence of disease; 5. Adequate hematological, hepatic and renal function; 6. Resolution of toxic effect(s) of prior anti-cancer therapy to ≤Grade 1 (Patients with ≤Grade 2 neuropathy, ≤Grade 2 endocrinopathy or ≤Grade 2 alopecia are exceptions); and 7. Effective contraception for women of child-bearing potential as defined by World Health Organization guidelines for 1 “highly effective” method or 2 “effective” methods.

Additional Phase 1 Monotherapy and Phase 1b Combination With Nivolumab Expansion Inclusion Criteria include: 1. Has one of the following tumor types: melanoma, non-small cell lung cancer, or triple negative breast cancer; and 2. Agrees to undergo a pre-treatment biopsy and another biopsy while on treatment.

Expansion Inclusion Criteria specific to Melanoma include: 1. Histologically confirmed, unresectable Stage III or Stage IV melanoma, as specified in the American Joint Committee on Cancer staging system; 2. Participants with ocular or uveal melanoma are ineligible; 3. PD-L1 status must be documented if available; 4. BRAF (V600) mutation status must be known. Both BRAF-mutated and wild type participants are permitted in this cohort; 5. BRAF-mutated participants must have been treated with approved targeted therapies; 6. Must have documented progressive or recurrent disease on or after discontinuation of anti-PD-(L)1 therapy (administered as monotherapy or as part of a combination) as per RECIST 1.1 criteria; and 7. Participants who received anti-PD-(L)1 in the adjuvant setting must have documented progressive or recurrent disease on or within 6 months of discontinuation of anti-PD-(L)1 therapy (administered as monotherapy or as part of a combination) as per RECIST 1.1 criteria.

Expansion Inclusion Criteria specific to NSCLC include: 1. Histologically confirmed NSCLC meeting stage criteria for stage IIIB, stage IV, or recurrent disease; 2. Participants must have recurrent or progressive disease during or after platinum doublet-based chemotherapy or at least two prior lines of systemic therapy for advanced or metastatic disease OR Must have recurrent or progressive disease within 6 months after completing platinum-based chemotherapy for local disease; 3. Participants must have received and progressed on or after anti-PD-(L)1 therapy, if available; and 4. Status for actionable mutations (e.g., EGFR, ALK, ROS1, RET, etc.) must be known (when testing is available as per country/region standard of care practices); participants with actionable mutations must have received and progressed on, have been intolerant to, or not be a candidate for, standard tyrosine kinase inhibitors (as available per country/region standard of care practices).

Expansion Inclusion Criteria specific to TNBC include: 1. Histologically confirmed unresectable, locally advanced or metastatic triple negative breast cancer.PD-L1 status, HER2-negative, estrogen receptor-negative, and progesterone receptor-negative status must be evaluated by local institutions before enrolment per guidelines of the American Society of Clinical Oncology and the College of American Pathologists; 2. Patients must not have received an anti PD-1/PD-L1 for the treatment of the metastatic disease, but the administration of an anti PD-1/PD-L1 in the adjuvant setting is acceptable; 3. Patients must have received one line of chemotherapy for the treatment of their metastatic disease, and experience progression during or after that line of chemotherapy; and 4. Patients must have not received more than one line of chemotherapy for the treatment of their unresectable, recurrent or metastatic disease.

Inclusion Criteria for Phase 2 (General) include: 1. Male or female patients aged ≥18 years; 2. ECOG performance status of 0 or 1; 3. Clinical or radiological evidence of measurable disease; 4. Adequate hematological, hepatic and renal function; 5. Resolution of toxic effect(s) of prior anti-cancer therapy to ≤Grade 1. (Patients with ≤Grade 2 neuropathy, ≤Grade 2 endocrinopathy or ≤Grade 2 alopecia are exceptions); 6. Participants must have received and progressed on or after anti-PD-(L)1 therapy; and 7. Effective contraception for women of child-bearing potential as defined by World Health Organization guidelines for 1 “highly effective” method or 2 “effective” methods.

Additional Inclusion Criteria for Phase 2 (Advanced Melanoma Patients) include: 1. Participants who received anti-PD-(L)1 in the advanced/metastatic setting, must have documented progressive or recurrent disease on or within 3 months of discontinuation of anti-PD-(L)1 therapy; 2. Participants who received anti-PD-(L)1 in the adjuvant setting must have documented progressive or recurrent disease on or within 6 months of discontinuation of anti-PD-(L)1 therapy; 3. Disease progression was confirmed at least 4 weeks after the initial diagnosis of disease progression while receiving an anti PD-1 antibody; 4. BRAF mutation status must be known and treated with approved targeted therapies; 5. Received a BRAF inhibitor if the tumor carries a BRAF activating mutation and progressed after the last line of treatment; 6. Participants with ocular or uveal melanoma are ineligible; and 7. Confirmation of radiographic progression on prior anti-PD-(L)1 therapy is required with a scan confirming progression at least 4 weeks after the initial progression.

Additional Inclusion Criteria for Phase 2 (Non-small Cell Lung Cancer) include: 1. Participants must have recurrent or progressive disease during or after platinum doublet-based chemotherapy or at least two prior lines of systemic therapy for advanced or metastatic disease OR must have recurrent or progressive disease within 6 months after completing platinum-based chemotherapy for local disease; and 2. Status for actionable mutations must be known; participants with actionable mutations must have received and progressed on, have been intolerant to, or not be a candidate for, standard tyrosine kinase inhibitors.

Exclusion Criteria for All Patients (All Phases): 1. Prior treatment with rhIL2 or any recombinant long acting drug containing an IL2 moiety; 2. Concurrent anticancer treatment (with the exception of palliative bone directed radiotherapy), immune therapy, or cytokine therapy (except for erythropoietin), major surgery (excluding prior diagnostic biopsy), concurrent systemic therapy with steroids or other immunosuppressive agents, or use of any investigational drug within 28 days before the start of study treatment; 3. Previous malignant disease other than the current target malignancy within the last 3 years, with the exception of basal or squamous cell carcinoma of the skin, localized prostate cancer or cervical carcinoma in situ; 4. Rapidly progressive disease; 5. Any Grade 2 and higher neurological or pulmonary toxicity during a treatment with an anti-PD-1 or PD-L1 agent administered as a monotherapy; 6. Active or history of central nervous system (CNS) metastases unless all of the following criteria are met: a. CNS lesions are asymptomatic and previously treated; b. Patient does not require ongoing steroid treatment daily for replacement for adrenal insufficiency; c. Imaging demonstrates stability of disease 28 days from last treatment for CNS metastases; 7. Receipt of any organ transplantation, autologous or allogeneic stem-cell transplantation; 8. Significant acute or chronic infections, or active or latent hepatitis B or active hepatitis C; 9. Preexisting autoimmune disease needing treatment with systemic immunosuppressive agents for more than 28 days within the last 3 years, clinically relevant immunodeficiencies, or fever within 7 days of Day 1; 10. Known severe hypersensitivity reactions to monoclonal antibodies and any history of anaphylaxis, or uncontrolled asthma; 11. Serious cardiac illness or medical conditions; and 12. History of life-threatening toxicity related to prior immune therapy except those that are unlikely to re-occur with standard countermeasures.

### Exploratory Objectives

The exploratory objectives, both with DF hIL12-Fc si as monotherapy and in combination with Nivolumab, are to: evaluate changes from baseline in tumor and peripheral biomarkers, and the relationship to PK; assess the PK of Nivolumab (Phase 1b and Cohort 2C only); evaluate the activity of DF hIL12-Fc si in the Efficacy Expansion Cohorts Part (Phase 2) per Investigator assessment (ORR, DOR, CBR, and BOR, by RECIST); evaluate the association between tumor and peripheral biomarkers, and tumor response rate.

### Study Design Overview

This study is a Phase 1/2, open-label, dose-escalation study with a consecutive parallel-group efficacy expansion study, designed to determine the safety, tolerability, PK, pharmacodynamics, and preliminary anti-tumor activity of DF hIL12-Fc si as monotherapy and in combination with Nivolumab. A schematic diagram of the study design is shown in FIG. 52A (for monotherapy) and 52B (for combination therapy with Nivolumab).

The study consists of 3 parts: Phase 1: Dose-escalation as a monotherapy using a 3+3 design, with Phase 1 Cohort Expansion; Phase 1b: Dose-escalation as a combination with Nivolumab using a 3+3 design, with Phase 1b Cohort Expansion; Phase 2: Efficacy Expansion using a group sequential design.

DF hIL12-Fc si is evaluated as a monotherapy in Efficacy Expansion cohorts in the following indications: Cohort 2A: Advanced (unresectable or metastatic) melanoma; Cohort 2B: Advanced (unresectable or metastatic) renal cell carcinoma (RCC).

DF hIL12-Fc si is evaluated in combination with Nivolumab in an Efficacy Expansion cohort in the following indication: Cohort 2C: Advanced (unresectable or metastatic) urothelial carcinoma.

In each study phase, patients receive DF hIL12-Fc si on Day 1 every 4 weeks (Q4W). Patients receive DF hIL12-Fc si until confirmed progressive disease (PD), unacceptable toxicity (i.e., dose-limiting toxicity [DLT]), or any reason for withdrawal from the study or Investigational Medicinal Product (IMP) occurs.

Phase 1 Dose Escalation DF hIL12-Fc si Monotherapy

The Phase 1 Dose-escalation Phase of the study is designed to determine the dose-limiting toxicities (DLTs) and maximum tolerated dose (MTD) of DF hIL12-Fc si as monotherapy using a standard 3+3 design.

The decision to escalate to the next dose level (DL) is based on safety assessments after all patients of a cohort have had safety evaluations performed through Cycle 1, Day 21 (C₁D21), unless due to DLT. In order to assess the safety of DF hIL12-Fc si, a Safety Monitoring Committee (SMC), responsible for dose-escalation decisions, is established.

After the safety of Dose Level “n” has been established, the SMC has the option to permit enrollment in the Phase I expansion cohort up to that dose level; no more than 50 patients can be enrolled by this process.

The MTD is defined as the highest DL at which ≤1 patient of 6 evaluable patients experiences a DLT.

Phase 1b: Dose-Escalation as a Combination with Nivolumab

The Phase 1b Dose-escalation Phase of the study is designed to determine the DLTs and MTD of DF hIL12-Fc si when given in combination with nivolumab, using a standard 3+3 design, as described for Phase 1.

Nivolumab is administered once every 4 weeks (on Day 1 of each cycle) per its U.S. package insert. The administration of nivolumab precedes that of DF hIL12-Fc si.

DF hIL12-Fc si dose levels tested in combination with Nivolumab are the same as those tested as a monotherapy.

Phase 1b starts after the SMC has established the safety of DL2 monotherapy (defined as the agreement to initiate enrollment into DL3). Phase 1b starts at a DF hIL12-Fc si dose at least 1 level below the safe dose established with monotherapy at the time the Phase 1b is initiated.

After the safety of Dose Level “n” has been established, the SMC has the option to permit enrollment in the Phase 1b Expansion Cohort; no more than 50 patients can be enrolled by this process across dose levels.

### Phase 2 Efficacy Expansion

The following tumor types are enrolled at the recommended phase 2 dose (RP2D): As a monotherapy: Cohort 2A: Advanced (unresectable or metastatic) melanoma; Cohort 2B: Advanced (unresectable or metastatic) renal cell carcinoma. In combination with Nivolumab, Cohort 2C: Advanced (unresectable or metastatic) urothelial carcinoma.

### Safety Oversight

Male or female patients aged ≥18 years with an Eastern Cooperative Oncology Group (ECOG) performance status of 0 or 1 at study entry and an estimated life expectancy of at least 3 months are enrolled. Key inclusion criteria in each study phase/cohort are as follows: Dose-Escalation cohorts in Phase 1/1b: histologically or cytologically proven locally advanced or metastatic solid tumors for which no standard therapy exists or for which standard therapy has failed in the following indications: melanoma, non-small cell lung (NSCLC), small cell lung, head and neck squamous cell, urothelial, gastric, esophageal, cervical, hepatocellular, merkel cell, cutaneous squamous cell carcinoma, renal cell, endometrial, triple negative breast (TNBC), ovarian, and prostate cancers; clinical or radiological evidence of disease.

Dose Expansion Cohorts in Phase 1/1b: histologically or cytologically proven locally advanced or metastatic solid tumors for which no standard therapy exists or for which standard therapy has failed; Has measurable disease, as determined by the Investigator using the Response Evaluation Criteria for Solid Tumors (RECIST), version 1.1.

### Cohort 2A

Patients with advanced melanoma who: received treatment with an anti-programmed cell death protein 1 (PD-1) antibody for at least 6 weeks; have a confirmation of PD at least 4 weeks after the initial diagnosis of PD while receiving an anti PD-1 is made. Confirmation of PD can be based on radiological or clinical observations; must have received a BRAF inhibitor if the tumor carries a BRAF activating mutation and have progressed after the last line of treatment.

### Cohort 2B

Patients with advanced RCC who: have any clear cell histology component; received treatment with an anti PD-1/PD-L1 antibody and an anti-vascular endothelial growth factor therapy as a monotherapy or in combination; received ≤3 prior lines of therapy.

### Cohort 2C

Patients with advanced urothelial carcinoma who: have histologically or cytologically documented locally advanced or metastatic transitional cell carcinoma of the urothelium (including renal pelvis, ureters, urinary urothelial, urethra); have received one (and no more than one) platinum-containing regimen (e.g., platinum plus another agent such as gemcitabine, methotrexate, vinblastine, doxorubicin, etc.) for inoperable locally advanced or metastatic urothelial carcinoma with radiographic progression or with recurrence within 6 months after the last administration of a platinum-containing regimen as an adjuvant, which would be considered failure of a first-line, platinum-containing regimen; have received no more than 2 lines of therapy (including the platinum-containing regimen) for the treatment of the metastatic disease; have not received treatment with a checkpoint inhibitor (CPI) (i.e., anti-PD-1 or anti-PD-L1 as a monotherapy or in combination with a platinum-based chemotherapy.

Dose Mode of Administration Dosing Schedule

DF hIL12-Fc si is administered as a subcutaneous (SC) injection Q4W (i.e., on Day 1 of each cycle) in both monotherapy and combination cohorts. Patients receive the drug SC in a volume of not more than 1 mL in a maximum of 2 injection sites. The second administration is completed within 10 minutes after the completion of the first administration, if applicable.

In Phase 1/1b, patients are hospitalized for the night following the first administration of DF hIL12-Fc si.

The DF hIL12-Fc si DLs (μg/kg) are as follows in Table 77.

TABLE 77
DF hIL12-Fc si DLs (μg/kg)

| | DL1 | DL2 | DL3 | DL4 | DL5 | DL6 | DL7 | DL8 | DL9 | DL10 |
|---|---|---|---|---|---|---|---|---|---|---|
| Dose of DF | 0.05 | 0.10 | 0.20 | 0.40 | 0.60 | 0.80 | 1.00 | 1.20 | 1.40 | 1.75 |
| hIL12-Fc si | | | | | | | | | | |
| (μ/kg) | | | | | | | | | | |
| Equivalent | 0.021 | 0.053 | 0.105 | 0.211 | 0.316 | 0.421 | 0.526 | 0.632 | 0.737 | 0.921 |
| IL12 | | | | | | | | | | |
| (μg/kg) | | | | | | | | | | |

The dose of DF hIL12-Fc si is calculated based on the weight of the patient at baseline. The patient's calculated dose is only recalculated if the patient's weight changes by 10% or more since the time of their last dose calculation.

In Phase 1b and Cohort 2C, Nivolumab is administered at a dose of 480 mg, once every 4 weeks (Q4W) via intravenous (IV) infusion, in accordance with the package insert. The administration of nivolumab precedes that of DF hIL12-Fc si. DF hIL12-Fc si is administered within 1 hour after the completion of the administration of nivolumab.

### Efficacy Expansion as a Monotherapy (Cohorts 2A and 2B)

The primary endpoint for this phase is the ORR. For each of these cohorts, the null hypothesis is that the objective response rate (ORR) does not exceed 5% (H0: ORR<5%) and the alternative hypothesis is that the ORR is greater than 5% (H1: ORR≥5%).

The target ORR of DF hIL12-Fc si as a monotherapy is 20%. It is expected to enroll 40 patients for each of these cohorts (i.e., approximately 80 patients in total).

Using a group sequential design, with 40 patients in each of the indication cohorts, the efficacy cohort provides ˜90% study power to detect a 15% difference at a 1-sided overall type I error rate of 0.025, assuming the target ORR of 20% for DF hIL12-Fc si.

For each of Cohorts 2A and 2B, a futility interim analysis, with Lan-DeMets O'Brien Fleming boundary, is planned at 50% information fraction (i.e., at ˜20 patients).

Efficacy Expansion in Combination with Nivolumab (Cohort 2C)

The Phase 2 portion for efficacy expansion in combination with nivolumab determines the clinical activity of DF hIL12-Fc si in combination in patients with UBC who have progressed after one line of platinum-based chemotherapy.

The study enrolls 40 patients so that the observation of 14 responses (CR or PR) out of the 40 patients enroll will lead to a 95% CI (0.206; 0.517) that excludes the value of the percentage of responses reported for nivolumab in a similar population, that was enrolled in Checkmate 275. In that study, the ORR was 19.6% (Sharma P, Retz M, Siefker-Radtke A, Baron A, Necchi A, Bedke J, et al. Nivolumab in metastatic urotheilal carcinoma after platin therapy (CheckMate 275): a multicentre, single-arm phase 2 trial. Lancet Oncol. 2017 Jan. 25; S1470-2045(17):30065-7).

### Exploratory Biomarkers

### Peripheral Biomarkers

Peripheral biomarkers are assessed in the periphery in all patients, including: cellular parameters: peripheral blood mononuclear cell (PBMCs) for immunophenotyping (IPT) by flow cytometry; soluble factors: Cytokines and chemokines in serum samples; ex vivo IL12 response assay: PBMCs for ex vivo stimulation followed by analysis of IFNγ production; circulating tumor (ct) deoxyribonucleic acid (DNA).—Gene expression profile performed using Nanostring: total RNA isolated from peripheral blood collected during screening and on C1D15; IPT assessments are performed on PBMCs derived from whole blood samples collected 2 hours prior to administration of DF hIL12-Fc si C1 through C3 and at each of the following study visits: C1D3, C1D8, C2D8, and C3D3; Soluble factors are determined in serum samples collected within 2 hours prior to DF hIL12-Fc si administration on D1 of each treatment cycle, and on C1D2, C1D3, C1D5, C1D8, C1D15, C2D3, C3D3, and C4D3, and at the EOT and SFU visits.

In order to complete all the assessments on tumor materials, blood (e.g., whole blood, plasma, and serum samples), is collected from patients.

### Biomarkers Derived From Tumor Tissue

Tissue derived biomarkers are evaluated on the pre-treatment and on treatment biopsies in patients participating in the Dose-escalation phase (optional biopsies), the Phase 1/1b Expansion Cohorts part (mandatory biopsies), and the Phase 2 Efficacy Expansion Cohorts phase (mandatory biopsies).

A panel of putative markers including molecular, soluble and cellular markers is analyzed at baseline from archived tumor tissue (or fresh tumor biopsy, if available), whole blood, and serum samples to investigate a possible correlation between clinical efficacy and analyzed markers.

For patients enrolled in the Dose-escalation Phase, the level of PD-L1 expression is determined using a commercially available kit (Dako PD-L1 IHC 22C3 pharmDx) and analysis of CD3 positivity (T cell infiltration) is determined by immunohistochemistry (IHC).

For patients enrolled in the Phase 1/1b Expansion Cohorts and the Efficacy Expansion Cohorts, fresh mandatory tumor biopsies are performed at Screening (i.e., within 30 days before first study drug dose) and at pre-specified time points during the treatment period.

Other biomarkers that are assessed include: frequency and localization of tumor-infiltrated leukocytes (e.g., CD8, CD4 T-cells, Treg, NK cells, macrophage [M1/2 profile] by IHC or IF), gene expression profile, and pharmacogenomics (PGx).

Germline DNA may be investigated on DNA extracted from whole blood and/or archival tumors. This extracted DNA is used for Whole Exome Sequencing and/or genotyping. For this purpose, an additional 6 mL of whole blood is collected at baseline (i.e., prior to the first administration of study treatment) for all indications; no additional tumor samples are needed because a part of the archived tumor sample is used for the extraction of DNA to study tumor genetics if required.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A pharmaceutical formulation comprising:
(a) a heterodimeric Fc-fused protein comprising:
(i) a first polypeptide comprising a first antibody Fc domain polypeptide and a first subunit of a multisubunit cytokine; and
(ii) a second polypeptide comprising a second antibody Fc domain polypeptide and a second, different subunit of the multisubunit cytokine,
(b) citrate;
(c) a sugar;
(d) a sugar alcohol; and
(e) a non-ionic surfactant,
at pH 6.0 to 7.0,
wherein the first and second antibody Fc domain polypeptides each comprise different mutations promoting heterodimerization, and
wherein the first subunit and second, different subunit of the multisubunit cytokine are bound to each other.

2. The pharmaceutical formulation of claim 1, wherein the first and/or second antibody Fc domain polypeptides comprise one or more mutation(s) that reduce(s) an effector function of an Fc.

3. The pharmaceutical formulation of claim 1 or 2, wherein the concentration of citrate in the pharmaceutical formulation is about 10 mM to about 30 mM.

4. The pharmaceutical formulation of claim 3, wherein the concentration of citrate in the pharmaceutical formulation is about 20 mM.

5. The pharmaceutical formulation of any one of claims 1-4, wherein the concentration of the sugar in the pharmaceutical formulation is about 3% to about 12% (w/v).

6. The pharmaceutical formulation of claim 5, wherein the concentration of the sugar in the pharmaceutical formulation is about 6% (w/v).

7. The pharmaceutical formulation of claim 5 or 6, wherein the sugar is a disaccharide.

8. The pharmaceutical formulation of claim 7, wherein the disaccharide is sucrose.

9. The pharmaceutical formulation of any one of claims 1-8, wherein the concentration of the sugar alcohol in the pharmaceutical formulation is between about 0.5% to about 6% (w/v).

10. The pharmaceutical formulation of claim 9, wherein the concentration of the sugar alcohol in the pharmaceutical formulation is about 1% (w/v).

11. The pharmaceutical formulation of any one of claims 1-10, wherein the sugar alcohol is derived from a monosaccharide.

12. The pharmaceutical formulation of claim 11, wherein the sugar alcohol is mannitol.

13. The pharmaceutical formulation of any one of claims 1-12, wherein the concentration of the non-ionic surfactant in the pharmaceutical formulation is between about 0.005% to about 0.02% (w/v).

14. The pharmaceutical formulation of claim 13, wherein the concentration of polysorbate 80 in the pharmaceutical formulation is about 0.01% (w/v).

15. The pharmaceutical formulation of claim 13 or 14 wherein the non-ionic surfactant is a polysorbate.

16. The pharmaceutical formulation of claim 15, wherein the polysorbate is polysorbate 80.

17. The pharmaceutical formulation of any one of claims 1-16, wherein the pH is between about 6.1 and about 6.9.

18. The pharmaceutical formulation of claim 17, wherein the pH is between about 6.2 and about 6.8.

19. The pharmaceutical formulation of claim 18, wherein the pH is between about 6.3 and about 6.7.

20. The pharmaceutical formulation of claim 19, wherein the pH is between about 6.4 and about 6.6.

21. The pharmaceutical formulation of claim 20, wherein the pH is about 6.5.

22. The pharmaceutical formulation of any one of claims 1-21, further comprising water.

23. The pharmaceutical formulation of claim 22, wherein the water is Water for Injection, USP.

24. The pharmaceutical formulation of any one of claims 1-23, wherein the pharmaceutical formulation comprises a bulk concentration of heterodimeric Fc-fused protein of about 1 g/L to about 10 g/L.

25. The pharmaceutical formulation of claim 24, wherein the pharmaceutical formulation comprises a bulk concentration of heterodimeric Fc-fused protein of about 2 g/L to about 8 g/L.

26. The pharmaceutical formulation of claim 25, wherein the pharmaceutical formulation comprises a bulk concentration of heterodimeric Fc-fused protein of about 4 g/L to about 6 g/L.

27. The pharmaceutical formulation of claim 26, wherein the pharmaceutical formulation comprises a bulk concentration of heterodimeric Fc-fused protein of about about 5 g/L.

28. The pharmaceutical formulation of any one of claims 1-27, wherein the pharmaceutical formulation comprises a concentration of the protein for administration of about 0.5 g/L to about 1.5 g/L.

29. The pharmaceutical formulation of claim 28, wherein the pharmaceutical formulation comprises a concentration of the protein for administration of about 0.75 g/L to about 1.25 g/L.

30. The pharmaceutical formulation of claim 29, wherein the pharmaceutical formulation comprises a concentration of the protein for administration of about 1 g/L.

31. The pharmaceutical formulation of any one of 1-30, wherein the formulation is designed to be stored at a temperature between about 2° C. and about 8° C.

32. The pharmaceutical formulation of any one of claims 1-31, wherein the pharmaceutical formulation is a clear, colorless solution and free of visible particulates.

33. The pharmaceutical formulation of any one of claims 1-32, wherein the formulation has a thermal stability profile as defined by:
(a) a Tₘ₁ of greater than about 60° C., greater than about 61° C., greater than about 62° C., greater than about 63° C., greater than about 64° C., greater than about 65° C., or greater than about 66° C.; and/or
(b) a Tₘ₂ of greater than about 70° C., greater than about 71° C., greater than about 72° C., greater than about 73° C., greater than about 74° C., greater than about 75° C., greater than about 76° C., or greater than about 77° C.,
as measured by differential scanning fluorimetry.

34. The pharmaceutical formulation of claim 33, wherein the formulation has a thermal stability profile as defined by a Tₘ₁ of about 67.0° C. and a Tₘ₂ of about 77.3° C.

35. The pharmaceutical formulation of claim 34, wherein the thermal stability profile of the pharmaceutical formulation, as defined by Tₘ₁ and/or Tₘ₂ is changed by less than about 2° C. or less than about 1° C. when the pharmaceutical formulation is incubated for 1 week at 50° C., as compared to the same pharmaceutical formulation that is incubated for 1 week at 5° C., as measured by differential scanning fluorimetry.

36. The pharmaceutical formulation of any one of 1-35, wherein the formulation has a thermal stability profile as defined by a T_{agg} of greater than about 60° C., greater than about 61° C., greater than about 62° C., greater than about 63° C., greater than about 64° C., greater than about 65° C., greater than about 66° C., or greater than about 67° C., as measured by differential scanning fluorimetry.

37. The pharmaceutical formulation of claim 36, wherein the thermal stability profile of the pharmaceutical formulation, as defined by T_{agg} is changed by less than about 2° C. or less than about 1° C. when the pharmaceutical formulation is incubated for 1 week at 50° C., as compared to the same pharmaceutical formulation that is incubated for 1 week at 5° C., as measured by differential scanning fluorimetry.

38. The pharmaceutical formulation of any one of claims 1-37, wherein the pH of the pharmaceutical formulation does not change by more than about 0.2 or about 0.1 in pH value after the pharmaceutical formulation is incubated for 1 week at 5° C.

39. The pharmaceutical formulation of any one of claims 1-38, wherein the pH of the pharmaceutical formulation does not change by more than about 0.2 or about 0.1 in pH value after the pharmaceutical formulation is incubated for 1 week at 50° C.

40. The pharmaceutical formulation of any one of claims 1-39, wherein the heterodimeric Fc-fused protein in the pharmaceutical formulation has a Z-average hydrodynamic diameter of less than about 15 nm, less than about 14 nm, less than about 13 nm, or less than about 12 nm, as measured by dynamic light scattering at 25° C.

41. The pharmaceutical formulation of claim 40, wherein the heterodimeric Fc-fused protein in the pharmaceutical formulation has a Z-average hydrodynamic diameter of about 11.6 nm.

42. The pharmaceutical formulation of any one of claims 1-41, wherein the heterodimeric Fc-fused protein in the pharmaceutical formulation has a Z-average hydrodynamic diameter of less than about 20 nm, less than about 19 nm, less than about 18 nm, less than about 17 nm, less than about 16 nm, or less than about 15 nm, as measured by dynamic light scattering at 25° C., after the pharmaceutical formulation is incubated for 2 weeks at 50° C.

43. The pharmaceutical formulation of claim 42, wherein the heterodimeric Fc-fused protein in the pharmaceutical formulation has a Z-average hydrodynamic diameter of about 14.4 nm.

44. The pharmaceutical formulation of any one of claims 1-43, wherein the heterodimeric Fc-fused protein in the pharmaceutical formulation has a Z-average hydrodynamic diameter of less than about 20 nm, less than about 19 nm, less than about 18 nm, less than about 17 nm, or less than about 16 nm, as measured by dynamic light scattering at 25° C., after the pharmaceutical formulation is subjected to five freeze thaw cycles.

45. The pharmaceutical formulation of claim 44, wherein the heterodimeric Fc-fused protein in the pharmaceutical formulation has a Z-average hydrodynamic diameter of about 15.3 nm.

46. The pharmaceutical formulation of any one of claims 1-45, wherein the polydispersity index of the heterodimeric Fc-fused protein in the pharmaceutical formulation is less than about 0.30, less than about 0.29, less than about 0.28, or less than about 0.27, as measured by dynamic light scattering at 25° C.

47. The pharmaceutical formulation of any one of claims 1-46, wherein the polydispersity index of the heterodimeric Fc-fused protein in the pharmaceutical formulation is about 0.26.

48. The pharmaceutical formulation of any one of claims 1-47, wherein the polydispersity index of the heterodimeric Fc-fused protein in the pharmaceutical formulation is less than about 0.30, less than about 0.29, less than about 0.28, less than about 0.27, or less than about 0.26, as measured by dynamic light scattering at 25° C., after the pharmaceutical formulation is incubated for 2 weeks at 50° C.

49. The pharmaceutical formulation of any one of claims 1-48, wherein the polydispersity index of the heterodimeric Fc-fused protein in the pharmaceutical formulation is about 0.25.

50. The pharmaceutical formulation of any one of claims 1-49, wherein the polydispersity index of the heterodimeric Fc-fused protein in the pharmaceutical formulation is less than about 0.40, less than about 0.35, or less than about 0.34, as measured by dynamic light scattering at 25° C., after the pharmaceutical formulation is subjected to five freeze thaw cycles.

51. The pharmaceutical formulation of any one of claims 1-50, wherein the polydispersity index of the heterodimeric Fc-fused protein in the pharmaceutical formulation is about 0.33.

52. The pharmaceutical formulation of any one of claims 1-51, wherein the purity profile of the pharmaceutical formulation, as measured by the area of the main peak as a percentage of total detected area in a SEC-HPLC analysis, is greater than about 90%, greater than about 91%, greater than about 92%, greater than about 93%, greater than about 94%, greater than about 95%, greater than about 96%, greater than about 97%, greater than about 98%, or greater than about 99%.

53. The pharmaceutical formulation of claim 52, wherein the purity profile of the pharmaceutical formulation, as measured by the area of the main peak as a percentage of total detected area in a SEC-HPLC analysis, is about 99.0%.

54. The pharmaceutical formulation of any one of claims 1-53, wherein the purity profile of the pharmaceutical formulation, as measured by the area of the main peak as a percentage of total detected area in a SEC-HPLC analysis, is greater than about 75%, greater than about 80%, greater than about 81%, greater than about 82%, greater than about 83%, greater than about 84%, or greater than about 85%, after the pharmaceutical formulation is incubated for 2 weeks at 50° C.

55. The pharmaceutical formulation of claim 54, wherein the purity profile of the pharmaceutical formulation, as measured by the area of the main peak as a percentage of total detected area in a SEC-HPLC analysis, is about 85.2%.

56. The pharmaceutical formulation of any one of claims 1-55, wherein the purity profile of the pharmaceutical formulation, as measured by the area of the main peak as a percentage of total detected area in a SEC-HPLC analysis, is greater than about 90%, greater than about 91%, greater than about 92%, greater than about 93%, greater than about 94%, greater than about 95%, greater than about 96%, greater than about 97%, or greater than about 98%, after the pharmaceutical formulation is subjected to five freeze thaw cycles.

57. The pharmaceutical formulation of claim 56, wherein the purity profile of the pharmaceutical formulation, as measured by the area of the main peak as a percentage of total detected area in a SEC-HPLC analysis, is about 98.9%.

58. A method comprising administering to a subject in need thereof, the pharmaceutical formulation of any one of claims 1-57, as a single-dose therapy.

59. A method comprising administering to a subject in need thereof, the pharmaceutical formulation of any one of claims 1-57, in a multiple-dose therapy at an interval of at least three weeks between the doses or at least four weeks between the doses.

60. The method of claim 59, wherein the pharmaceutical formulation is administered to the subject once every three weeks.

61. The method of claim 59, wherein the pharmaceutical formulation is administered to the subject once every four weeks.

62. The method of claim 59, wherein the pharmaceutical formulation is administered to the subject once every six weeks.

63. The method of any one of claims 59-62, further comprising stopping the multi-dose therapy if the subject develops progressive disease, unacceptable toxicity, or meets a criterion for withdrawal.

64. The method of any one of claims 59-63, wherein if the subject experiences a complete response (CR) during the multi-dose therapy, then the multi-dose therapy is further administered for at least 12 months after the confirmation of the complete response.

65. The method of claim 64, wherein the total duration of the multi-dose therapy is equal to or less than 24 months.

66. The method of claim 64, wherein the total treatment duration is greater than 24 months.

67. The method of any one of claims 58-66, wherein the pharmaceutical formulation is administered by subcutaneous injection.

68. The method of any one of claims 58-67, wherein the pharmaceutical formulation is administered to the subject in an amount sufficient to provide the heterodimeric Fc-fused protein at a dosage of between about 0.05 μg/kg to about 1.75 μg/kg, based on the subject's weight.

69. The method of any one of claims 58-68, wherein the pharmaceutical formulation is administered to the subject in an amount sufficient to provide the heterodimeric Fc-fused protein at a dosage of about 0.05 μg/kg, about 0.10 μg/kg, about 0.20 μg/kg, about 0.40 μg/kg, about 0.60 μg/kg, about 0.80 μg/kg, about 1.00 μg/kg, about 1.20 μg/kg, about 1.40 μg/kg, or about 1.75 μg/kg, based on the subject's weight.

70. The method of any one of claims 58-67, wherein the pharmaceutical formulation is administered to the subject in an amount sufficient to provide the heterodimeric Fc-fused protein at a dosage of greater than 0.00 μg/kg and less than about 0.05 μg/kg, based on the subject's weight.

71. The method of any one of claims 58-67, wherein the pharmaceutical formulation is administered to the subject in an amount sufficient to provide the heterodimeric Fc-fused protein at a dosage of greater than about 1.75 μg/kg, based on the subject's weight.

72. The method of any one of claims 58-71, wherein the subject has cancer.

73. The method of claim 72, wherein the subject has a locally advanced or metastatic solid tumor.

74. The method of claim 72 or 73, wherein the presence of the cancer in the subject is confirmed using the Response Evaluation Criteria for Solid Tumors (RECIST), version 1.1.

75. The method of any one of claims 72-74, wherein the cancer is selected from the group consisting of: melanoma, non-small cell lung cancer (NSCLC), small cell lung cancer (SCLC), head and neck squamous cell carcinoma (HNSCC), classical Hodgkin lymphoma, primary mediastinal large B-Cell lymphoma, bladder cancer, urothelial carcinoma, micro-satellite instability high cancer, colorectal cancer, gastric cancer, oesophageal cancer, cervical cancer, hepatocellular carcinoma, Merkel cell carcinoma, renal cell carcinoma (RCC), endometrial carcinoma, cutaneous T cell lymphoma, and triple negative breast cancer.

76. The method of any one of claims 72-75, wherein the subject is anti-PD-1 refractory.

77. The method of claim 75, wherein the subject has melanoma.

78. The method of claim 77, wherein the subject has previously been treated with an anti-PD-1 antibody for at least 6 weeks.

79. The method of claim 78, wherein the subject has been confirmed of progression of disease at least 4 weeks after the initial diagnosis of progression of disease while receiving an anti-PD-1 antibody.

80. The method of claim 79, wherein progression of disease is confirmed by radiological or clinical observation.

81. The method of claim 77, wherein, if the subject has a tumor comprising a BRAF activating mutation, then the subject has previously been treated with a BRAF inhibitor.

82. The method of claim 75, wherein the subject has RCC.

83. The method of claim 82, wherein the RCC has clear cell histology.

84. The method of claim 82, wherein the patient has previously been treated with an anti-PD-1/PD-L1 antibody and/or an anti-vascular endothelial growth factor therapy.

85. The method of claim 82, wherein the subject has previously received three or fewer lines of therapy.

86. The method of claim 75, wherein the subject has urothelial carcinoma.

87. The method of claim 86, wherein the subject has locally advanced or metastatic transitional cell carcinoma of the urothelium.

88. The method of claim 86, wherein the subject has previously been treated with a single treatment comprising a platinum-containing regimen and has shown radiographic progression recurrence within 6 months after the last administration of the platinum-containing regimen.

89. The method of claim 86, wherein the subject has previously received two or less lines of therapy.

90. The method of claim 86, wherein the subject has not previously received a checkpoint inhibitor (e.g., anti-PD-1 or anti-PD-L1 antibody) therapy as a monotherapy or in combination with a platinum based chemotherapy.

91. The method of any one of claims 58-90, wherein the pharmaceutical formulation is administered to the subject as a monotherapy.

92. The method of any one of claims 58-90, wherein the pharmaceutical formulation is administered to the subject as a combination therapy.

93. The method of claim 92, further comprising administering to the subject an anti-PD-1 antibody.

94. The method of claim 93, wherein the anti-PD-1 antibody is pembrolizumab.

95. The method of claim 94, wherein pembrolizumab is administered intravenously.

96. The method of claim 94 or 95, wherein pembrolizumab is administered at a dose of 200 mg.

97. The method of any one of claims 94-96, wherein administration of pembrolizumab precedes each administration of the pharmaceutical formulation.

98. The method of claim 97, wherein the pharmaceutical formulation is administered within 1 hour after completion of administration of pembrolizumab.

99. The method of claim 92, wherein the anti-PD-1 antibody is nivolumab.

100. The method of claim 99, wherein nivolumab is administered intravenously.

101. The method of claim 99 or 100, wherein nivolumab is administered at a dose of 480 mg.

102. The method of any one of claims 99-101, wherein administration of nivolumab precedes each administration of the pharmaceutical formulation.

103. The method of claim 102, wherein the pharmaceutical formulation is administered within 1 hour after completion of administration of nivolumab.

104. The method of any one of claims 99-103, wherein the cancer is selected from the group consisting of: melanoma, NSCLC, SCLC, RCC, classical Hodgkin lymphoma, HNSCC, urothelial carcinoma, colorectal cancer, hepatocellular carcinoma, bladder cancer, and oesophageal cancer.

105. The method of claim 104, wherein the cancer is melanoma.

106. The method of claim 105, wherein the melanoma is unresectable.

107. The method of claim 104, wherein the cancer is colorectal cancer.

108. The method of claim 107, wherein the colorectal cancer is microsatellite instability-high (MSI-H) or mismatch repair deficient metastatic (dMMR) colorectal cancer.

109. The method of any one of claims 92-98, further comprising performing a surgical intervention to lyse cancer cells, remove a tumor, or debulk a tumor in the subject.

110. The method of claim 109, wherein the surgical intervention comprises cryotherapy.

111. The method of claim 109, wherein the surgical intervention comprises hyperthermic therapy.

112. The method of claim 109, wherein the surgical intervention comprises administering to the subject a radiotherapy.

113. The method of claim 112, wherein the radiotherapy is a stereotactic body radiation therapy (SBRT).

114. The method of any one of claims 92-113, further comprising administering to the subject an NK cell-targeting therapy.

115. The method of claim 114, wherein the subject is administered a multi-specific binding protein.

116. The method of any one of claims 92-115, further comprising administering to the subject a chimeric antigen receptor therapy.

117. The method of any one of claims 92-116, further comprising administering to the subject a cytokine therapy.

118. The method of any one of claims 92-117, further comprising administering to the subject an innate immune system agonist therapy.

119. The method of any one of claims 92-118, further comprising administering to the subject a chemotherapy.

120. The method of any one of claims 92-119, further comprising administering to the subject a targeted antigen therapy.

121. The method of any one of claims 92-120, further comprising administering to the subject an oncolytic virus therapy.

122. A method of detecting toxicity in a subject receiving a pharmaceutical formulation comprising measuring the concentration of C-reactive protein (CRP) in the subject's blood, wherein the pharmaceutical formulation comprises a heterodimeric Fc-fused protein and a pharmaceutically acceptable carrier, and wherein the heterodimeric Fc-fused protein comprises a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization.

123. The method of claim 122, wherein
(1) if the CRP concentration in the subject's blood is higher than a threshold CRP concentration, then the subject is identified as being at risk for developing an adverse drug reaction; and
(2) if the CRP concentration in the subject's blood is about the same or lower than the threshold C-reactive protein concentration, the subject is not identified as being at risk for developing an adverse drug reaction.

124. The method of claim 122, wherein if the CRP concentration in the subject's blood is higher than the threshold CRP concentration, then (1) the administration of the pharmaceutical formulation is paused; (2) the heterodimeric Fc-fused protein is administered at a lower dose; or
(3) a remedial action is taken to reduce or alleviate the formulation's toxicity effects in the subject.

125. The pharmaceutical formulation of any one of claims 1-57 or the method of any one of claims 58-124, wherein the first and second antibody Fe domain polypeptides are human IgG1 Fc domain polypeptides.

126. The pharmaceutical formulation or the method of claim 125, wherein the multisubunit cytokine is a human IL12.

127. The pharmaceutical formulation or the method of claim 126, wherein the human IgG1 Fc domain polypeptides comprise one or more mutation(s) that reduce(s) an effector function of an Fc.

128. The pharmaceutical formulation or the method of claim 127, wherein the first and second antibody Fe domain polypeptides comprise mutations selected from L234A, L235A or L235E, G237A, P329A, A330S, and P331S, numbered according to the EU numbering system.

129. The pharmaceutical formulation or the method of claim 128, wherein the first and second antibody Fc domain polypeptides each comprise mutations L234A, L235A, and P329A.

130. The pharmaceutical formulation or the method of claim 129, wherein the first subunit of a multisubunit cytokine is a p40 subunit of IL12 and the second subunit of a multisubunit cytokine is a p35 subunit of IL12.

131. The pharmaceutical formulation or the method of claim 130, wherein the first subunit of a multisubunit cytokine comprises the amino acid sequence of SEQ ID NO: 127 and the second subunit of a multisubunit cytokine comprises the amino acid sequence of SEQ ID NO: 128.

132. The pharmaceutical formulation or the method of claim 131, wherein the second subunit of a multisubunit cytokine is fused to the second antibody Fc domain by a linker comprising the amino acid sequence of SEQ ID NO: 108.

133. The pharmaceutical formulation or the method of claim 132, wherein
(a) the first antibody Fc domain comprises mutations L234A, L235A, P329A, Y349C, K360E, and K409W, and
(b) the second antibody Fc domain comprises mutations L234A, L235A, P329A, Q347R, S354C, D399V, and F405T.

134. The pharmaceutical formulation or the method of claim 133, wherein
(a) the first antibody Fc domain comprises the amino acid sequence of SEQ ID NO:215, and
(b) the second antibody Fc domain comprises the amino acid sequence of SEQ ID NO:216.

135. The pharmaceutical formulation or the method of claim 134, wherein the first antibody Fc domain peptide comprises the amino acid sequence of SEQ ID NO:290 and the second antibody Fc domain peptide comprises the amino acid sequence of SEQ ID NO:291.

136. A kit comprising one or more vessels comprising a pharmaceutical formulation, wherein the pharmaceutical formulation comprises:
(a) a heterodimeric Fc-fused protein comprising a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization; and
(b) a pharmaceutically acceptable carrier,
and wherein the one or more vessels collectively comprise about 0.1 mg-about 2 mg of heterodimeric Fc-fused protein.

137. The kit of claim 136, wherein the one or more vessels collectively comprise about 0.5 mg to about 2 mg of heterodimeric Fc-fused protein.

138. The kit of claim 137, wherein the one or more vessels collectively comprise about 1 mg of heterodimeric Fc-fused protein.

139. The kit of claim 138, wherein the kit comprises one vessel comprising about 1 mg of heterodimeric Fc-fused protein.

140. The kit of any one of claims 136-139, wherein the pharmaceutical formulation is a lyophilized formulation or a liquid formulation.

141. The kit of claim 140, wherein the pharmaceutical formulation is a liquid formulation supplied in a volume of 1 mL.

142. Use of a heterodimeric Fc-fused protein in the manufacture of a medicament for treating a cancer, wherein the medicament is manufactured in a liquid pharmaceutical formulation comprising about 0.5 g/L to about 1.5 g/L of the heterodimeric Fc-fused protein contained in one or more vessels,
wherein the heterodimeric Fc-fused protein comprises a first Fc region and a second Fc region of an immunoglobulin Fe (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fe regions, and wherein CH3 domains of the first Fc region and the second Fc region each comprise one or more mutations promoting heterodimerization.

143. The use of a heterodimeric Fc-fused protein of claim 142, wherein the liquid pharmaceutical formulation comprises about 1.0 g/L of the heterodimeric Fc-fused protein.

144. Use of a heterodimeric Fc-fused protein in the manufacture of a medicament for treating a cancer, wherein the medicament is manufactured in a liquid pharmaceutical formulation comprising about 0.1 mg-about 2 mg of heterodimeric Fc-fused protein contained in one or more vessels,
wherein the heterodimeric Fc-fused protein comprises a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization.

145. The use of a heterodimeric Fc-fused protein of claim 144, wherein the liquid pharmaceutical formulation comprises about 1 mg of heterodimeric Fc-fused protein.

146. The use of a heterodimeric Fc-fused protein of any one of claims 142-145, wherein the medicament is contained in one vessel.

147. The use of a heterodimeric Fc-fused protein of any one of claims 142-146, wherein each vessel contains 1 mg of heterodimeric Fc-fused protein.

148. The use of any one of claims 146-147, wherein the medicament is administered to the subject on day 1, every 3 weeks.

149. The use of any one of claims 146-147, wherein the medicament is administered to the subject on day 1, every 4 weeks.

150. The use of any one of claims 146-148, wherein the medicament is administered subcutaneously.

151. The use of any one of claims 146-150, wherein the medicament is administered in a volume of about 0.1 mL to about 1 mL.

152. The use of claim 151, wherein the medicament is administered in a volume of about 1 mL.

153. The use of any one of claims 146-152, wherein the medicament is administered to a maximum of two injection sites.

154. The use of claim 153, wherein a second injection is completed within 10 minutes after a first injection.

155. The use of any one of claims 146-154, wherein the medicament is administered at a dose of about 0.05 mg/kg to about 1.75 mg/kg.

156. The use of claim 155, wherein the medicament is administered at a dose of about 1 mg/kg.

157. The use of any one of claims 146-156, wherein the medicament is diluted prior to administration in a solution of 0.9% saline (sodium chloride for injection) and 0.01% polysorbate 80.

158. A method of manufacturing a heterodimeric Fc-fused protein for the preparation of a pharmaceutical formulation thereof, the method comprising adding acetic acid to a solution comprising the heterodimeric Fc-fused protein obtained from a Chinese Hamster Ovary (CHO) cell culture expressing the heterodimeric Fc-fused protein for 30 minutes to 90 minutes, wherein the acetate adjusts and maintains the pH of the solution at pH 3.55 to 3.75, and
wherein the heterodimeric Fc-fused protein comprises a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization.

159. The method of claim 158, wherein the acetic acid is added to the solution comprising the heterodimeric Fc-fused protein for about 60 minutes.

160. The method of claim 158 or 159, wherein the acetic acid adjusts and maintains the pH of the solution to about 3.65.

161. The method of claim 158, wherein the CHO cell culture expressing the heterodimeric Fc-fused protein is maintained in suspension.

162. The method of claim 161, wherein the CHO cell culture expressing the heterodimeric Fc-fused protein is cultured for 7-21 days in a bioreactor.

163. The method of claim 161 or 162, wherein the CHO cell culture expressing the heterodimeric Fc-fused protein is cultured for 14 days in a bioreactor.

164. The method of any one of claims 158-163, wherein the CHO cell culture expressing the heterodimeric Fc-fused protein is harvested by depth filtration to yield a CHO harvest medium.

165. The method of claim 164, wherein the depth filtration is a two-stage single-use depth filtration consisting of DOHC and XOHC filters.

166. The method of claim 164 or 165, wherein the heterodimeric Fc-fused protein is purified from the CHO harvest medium using Protein A capture chromatography, mixed mode chromatography, and cation exchange chromatography to yield the solution comprising the heterodimeric Fc-fused protein.

167. The method of claim 166, wherein Protein A capture chromatography comprises:
equilibrating a Protein A resin with 20 mM Tris, 150 mM NaCl at pH 7.5;
loading CHO harvest medium onto the Protein A resin;
washing the loaded Protein A resin with 20 mM Tris, 150 mM NaCl at pH 7.5;
washing the loaded Protein A resin with 50 mM acetate at pH 5.4; and
eluting the heterodimeric Fc-fused protein from the Protein A resin with 50 mM acetate, 100 mM arginine at pH 3.7 and collecting by 280 nm UV starting at 1.25 AU/cm ascending and ending at 1.25 AU/cm descending.

168. The method of claim 167, wherein the acetic acid is added at a concentration of 0.5M to the solution comprising the heterodimeric Fc-fused protein eluted from the Protein A resin, wherein the acetic acid acidifies the pH of the solution to pH 3.65 for 60 minutes, followed by neutralization of the solution to pH 5.2 by adding 2M Tris.

169. The method of claim 168, wherein following acidification and neutralization of the solution, the solution comprising the heterodimeric Fc-fused protein is passed through a 0.2 μm filter.

170. The method of claim 169, wherein the filtered solution comprising the heterodimeric Fc-fused protein eluted from the Protein A resin is passed through X0SP depth filtration.

171. The method of claim 170, wherein mixed mode chromatography comprises:
equilibrating a mixed mode chromatography column with 50 mM acetate at pH 5.2;
loading the solution passed through X0SP filtration onto the mixed mode chromatography column;
washing the loaded mixed mode chromatography column with 50 mM acetate at pH 5.2; and
eluting the heterodimeric Fc-fused protein from the mixed mode chromatography column with 50 mM Acetate, 250 mM NaCl at pH 5.2 and collecting by 280 nm UV starting at 0.625 AU/cm ascending and ending at 1.50 AU/cm descending.

172. The method of claim 171, wherein the solution comprising the heterodimeric Fc-fused protein eluted from the mixed mode chromatography column is passed through a 0.2 μm filter.

173. The method of claim 172, wherein cation exchange chromatography comprises:
equilibrating a cation exchange chromatography resin with 50 mM Tris at pH 7.4;
loading the filtered solution eluted from the mixed mode chromatography column onto the cation exchange chromatography resin;
washing the loaded cation exchange chromatography resin with 50 mM Tris at pH 7.4; and
eluting the heterodimeric Fc-fused protein from the cationic exchange chromatography resin with a gradient of 50 mM Tris at pH 7.4 and 50 mM Tris, 0.5 M NaCl at pH 7.4, and collecting by 280 nm UV starting at 2.5 AU/cm ascending and ending at 4.5 AU/cm descending.

174. The method of claim 173, wherein the solution comprising the heterodimeric Fc-fused protein eluted from the cation exchange chromatography resin is passed through a 0.2 μm filter.

175. The method of claim 174, wherein the filtered solution comprising the heterodimeric Fc-fused protein eluted from the cation exchange chromatography resin is nanofiltrated through a prefilter, a 20 nm nominal filter, and a 0.2 μm membrane.

176. The method of claim 175, wherein the nanofiltrated solution comprising the heterodimeric Fc-fused protein is ultrafiltrated and diafiltrated, wherein ultrafilitration and diafiltration comprises:
equilibrating an ultrafiltration system with 50 mM Tris, 265 mM NaCl at pH 7.4;
concentrating the nanofiltrated solution comprising the heterodimeric Fc-fused protein to a concentration of about 5.0 g/L;
exchanging the buffer using 7 diavolumes of 20 mM citrate at pH 6.5;
concentration the diafiltrated solution comprising the heterodimeric Fc-fused protein to a concentration of about 11.0 g/L;
diluting the concentration solution comprising the heterodimeric Fc-fused protein to a concentration of about 5 g/L to about 10 g/L with 20 mM citrate at pH 6.5; and
adding 20 mM citrate, 18% (w/v) sucrose, 3% (w/v) mannitol, 0.03% (w/v) polysorbate-80 at pH 6.5 to achieve a final concentration of the ultrafitration/diafiltration retentate solution comprising the heterodimeric Fc-fused protein of 20 mM citrate, 6% (w/v) sucrose, 1% (w/v) mannitol, 0.01% (w/v) polysorbate-80.

177. The method of claim 176, wherein the ultrafiltrated/diafiltrated solution comprising the heterodimeric Fc-fused protein is passed through a 0.2 μm membrane to yield a bulk drug substance.

178. The method of claim 177, wherein the bulk drug substance is diluted to an 80% drug product solution in a 0.2 μm filtered buffer comprising 20 mM citrate, 6% (w/v) sucrose, 1% (w/v) mannitol, and 0.01% (w/v) polysorbate-80 at pH 6.5.

179. The method of claim 177 or 178, wherein the bulk drug substance or 80% drug product is diluted to a concentration for administration of 1 mg/mL of the heterodimeric Fc-fused protein in a 0.2 μm filtered buffer comprising 20 mM citrate, 6% (w/v) sucrose, 1% (w/v) mannitol, and 0.01% (w/v) polysorbate-80 at pH 6.5.

180. A method of treating cancer in a subject who has received treatment with a checkpoint inhibitor antibody for at least 6 weeks, the method comprising administering a pharmaceutical formulation comprising a heterodimeric Fc-fused protein and a pharmaceutically acceptable carrier to the subject, wherein the heterodimeric Fc-fused protein comprises a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization.

181. The method of claim 180, wherein the checkpoint inhibitor antibody is an anti-programmed cell death protein 1 (PD-1) antibody.

182. The method of claim 180 or 181, wherein the cancer is melanoma.

183. The method of claim 182, wherein the melanoma is unresectable or metastatic.

184. The method of claim 182 or 183, wherein the subject is confirmed to have progressive disease at least 4 weeks after the initial diagnosis of progressive disease while receiving the anti-PD-1 antibody.

185. The method of any one of claims 182-184, wherein the subject is confirmed to have progressive disease at least 4 weeks after the initial diagnosis of progressive disease while receiving the anti-PD-1 antibody.

186. The method of any one of claims 184 or 185, wherein progressive disease is confirmed by radiological or clinical observation.

187. A method of treating cancer in a subject who has received treatment with a checkpoint inhibitor antibody or an anti-vascular endothelial growth factor therapy as a monotherapy, or in combination, the method comprising administering a pharmaceutical formulation comprising a heterodimeric Fc-fused protein and a pharmaceutically acceptable carrier to the subject, wherein the heterodimeric Fc-fused protein comprises a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization.

188. The method of claim 187, wherein the checkpoint inhibitor antibody is an anti-PD-1 antibody or an anti-PD-L1 antibody.

189. The method of claim 187 or 188, wherein the cancer is advanced renal cell carcinoma (RCC).

190. The method of claim 189, wherein the RCC is unresectable or metastatic.

191. The method of claim 189 or 190, wherein the RCC has a clear cell component.

192. The method of any one of claims 189-191, wherein the subject received no more than 3 previous lines of therapy.

193. The method of any one of claims 189-192, wherein the subject has not received treatment with a checkpoint inhibitor.

194. The method of claim 193, wherein the checkpoint inhibitor comprises an anti-PD-1 antibody or anti-PD-L1 antibody as a monotherapy or in combination with a platinum based chemotherapy.

195. A method of treating cancer in a subject who has received treatment with only one platinum-containing regimen, the method comprising administering a pharmaceutical formulation comprising a heterodimeric Fc-fused protein and a pharmaceutically acceptable carrier to the subject, wherein the heterodimeric Fc-fused protein comprises a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization.

196. The method of claim 195, wherein the platinum containing regimen is platinum in combination with an agent selected from gemcitabine, methotrexate, vinblastine, and doxorubicin.

197. The method of claim 195 or 196, wherein the cancer is locally advanced or metastatic transitional cell urothelial carcinoma.

198. The method of claim 197, wherein the urothelial carcinoma includes one or more of the group consisting of the renal pelvis, ureters, urinary urothelium, and urethra.

199. The method of claim 197 or 198, wherein the urothelial carcinoma is inoperable.

200. The method of any one of claims 197-199, wherein the urothelial carcinoma is characterized with radiographic progression or with recurrence within 6 months after the last administration of a platinum-containing regimen as an adjuvant.

201. The method of any one of claims 197-200, wherein the urothelial carcinoma is considered failure of a first-line, platinum-containing regimen.

202. The method of any one of claims 197-201, wherein the subject has received no more than 2 lines of therapy (including the platinum-containing regimen) for the treatment of the urothelial carcinoma prior to administration of the pharmaceutical formulation.

203. The method of any one of claims 197-202, wherein the subject has not received treatment with a checkpoint inhibitor (CPI) as a first-line therapy.

204. The method of claim 203, wherein the checkpoint inhibitor is an anti-PD-1 antibody or anti-PD-L1 antibody.

205. The method of claim 203 or 204, wherein the checkpoint inhibitor is a monotherapy or in combination with a platinum based chemotherapy.

206. The method of any one of claims 195-205, wherein the pharmaceutical formulation is administered in combination with pembrolizumab.

207. The method of claim 206, wherein pembrolizumab is administered once every 3 weeks.

208. The method of claim 206 or 207, wherein pembrolizumab is administered before administration of the pharmaceutical formulation.

209. The method of claim 208, wherein the pharmaceutical formulation is administered within one hour after the completion of administration of pembrolizumab.

210. The method of any one of claims 206-209, wherein pembrolizumab is administered at a dose of 200 mg.

211. The method of any one of claims 206-210, wherein pembrolizumab is administered intravenously.

212. The method of any one of claims 206-211, wherein the cancer is selected from the group consisting of: melanoma, non-small cell lung cancer (NSCLC), small cell lung cancer (SCLC), head and neck squamous cell carcinoma (HNSCC), classical Hodgkin lymphoma, primary mediastinal large B-cell lymphoma, urothelial carcinoma, microsatellite instability-high cancer, gastric cancer, oesophageal cancer, cervical cancer, hepatocellular carcinoma, Merkel cell carcinoma, renal cell carcinoma, and endometrial carcinoma.

213. The method of any one of claims 195-205, wherein the pharmaceutical formulation is administered in combination with nivolumab.

214. The method of claim 213, wherein nivolumab is administered before administration of the pharmaceutical formulation.

215. The method of claim 214, wherein the pharmaceutical formulation is administered within one hour after the completion of administration of nivolumab.

216. The method of any one of claims 213-215, wherein nivolumab is administered at a dose of about 480 mg.

217. The method of any one of claims 213-216, wherein nivolumab is administered intravenously.

218. The method of any one of claims 213-217, wherein the cancer is selected from the group consisting of: melanoma, non-small cell lung cancer (NSCLC), small cell lung cancer (SCLC), renal cell carcinoma, classical Hodgkin lymphoma, head and neck squamous cell carcinoma (HNSCC), colorectal cancer, hepatocellular carcinoma, bladder cancer, and oesophageal cancer.

219. The method of claim 218, wherein the cancer is melanoma.

220. The method of claim 219, wherein the melanoma is unresectable or metastatic.

221. The method of claim 218, wherein the cancer is colorectal cancer.

222. The method of claim 221, wherein the colorectal cancer is microsatellite instability-high (MSI-H) or mismatch repair deficient (dMMR) metastatic colorectal cancer.

223. The method of any one of claims 180-211, wherein the pharmaceutical formulation is administered to the subject on day 1, every 3 weeks.

224. The method of any one of claims 180-222, wherein the pharmaceutical formulation is administered to the subject on day 1, every 4 weeks.

225. The method of any one of claims 180-223, wherein the pharmaceutical formulation is administered subcutaneously.

226. The method of any one of claims 180-225, wherein the pharmaceutical formulation is administered in a volume of about 0.1 mL to about 1 mL.

227. The method of claim 226, wherein the pharmaceutical formulation is administered in a volume of about 1 mL.

228. The method of any one of claims 180-227, wherein the pharmaceutical formulation is administered to a maximum of two injection sites.

229. The method of claim 228, wherein a second injection is completed within 10 minutes after a first injection.

230. The method of any one of claims 180-229, wherein the pharmaceutical formulation is administered at a dose of about 0.05 mg/kg to about 1.75 mg/kg.

231. The method of claim 230, wherein the pharmaceutical formulation is administered at a dose of about 1 mg/kg.

232. The method of any one of claims 180-231, wherein the pharmaceutical formulation is diluted prior to administration in a solution of 0.9% saline (sodium chloride for injection) and 0.01% polysorbate 80.

233. The method of any one of claims 180-232, wherein the presence of the cancer is determined using the Response Evaluation Criteria for Solid Tumors (RECIST), version 1.1.

234. The method of any one of claims 180-233, wherein a subject who has a confirmed complete response is treated with the pharmaceutical formulation for at least 12 months after confirmation unless a criterion for discontinuation is met.

235. A method of treating a subject whose blood concentration of C-reactive protein (CRP) is monitored, the method comprising administering to the subject a pharmaceutical formulation comprising a heterodimeric Fc-fused protein and a pharmaceutically acceptable carrier, wherein the heterodimeric Fc-fused protein comprises a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization.

236. The method of claim 235, wherein
(1) if the CRP concentration in the subject's blood is higher than a threshold CRP concentration, then the subject is identified as being at risk for developing an adverse drug reaction; and
(2) if the CRP concentration in the subject's blood is about the same or lower than the threshold C-reactive protein concentration, the subject is not identified as being at risk for developing an adverse drug reaction.

237. The method of claim 235, wherein if the CRP concentration in the subject's blood is higher than the threshold CRP concentration, then (1) the administration of the pharmaceutical formulation is paused; (2) the heterodimeric Fc-fused protein is administered at a lower dose; or (3) a remedial action is taken to reduce or alleviate the formulation's toxicity effects in the subject.

238. A method of treating cancer in a subject in need thereof, the method comprising subcutaneous administration of a pharmaceutical formulation comprising a heterodimeric Fc-fused protein and pharmaceutically acceptable carrier to the subject,
wherein the heterodimeric Fc-fused protein comprises a first Fc region and a second Fc region of an immunoglobulin Fc (fragment crystallizable) pair and the p40 and p35 subunits of IL-12, wherein the p40 and p35 subunits of IL-12 are linked separately to the first Fc region and the second Fc region, or to the second Fc region and the first Fc region, respectively, wherein the p40 and p35 subunits are each linked to the N-terminus or C-terminus of the Fc regions, and wherein CH3 domains of the first Fc region and the second Fc region each comprise one or more mutations promoting heterodimerization; and the pharmaceutical formulation comprises citrate; a sugar; a sugar alcohol; and a non-ionic surfactant, and the pH of the formulation is between 5.5 and 7.0.

239. The kit of any one of claims 136-141, the use of any one of claims 142-157, or the method of any one of claims 158-238, wherein the first Fc region and second Fc region are human IgG1 Fc regions.

240. The kit, the use, or the method of claim 239, wherein human IgG1 Fc regions comprise one or more mutation(s) that reduce(s) an effector function of an Fc.

241. The kit, the use, or the method of claim 240, wherein the first Fc region and second Fc region comprise one or more mutation(s) selected from L234A, L235A or L235E, G237A, P329A, A330S, and P331S, numbered according to the EU numbering system.

242. The kit, the use, or the method of claim 241, wherein the first Fc region and second Fc region each comprise mutations L234A, L235A, and P329A.

243. The kit, the use, or the method of claim 242, wherein the p40 subunit of IL12 comprises the amino acid sequence of SEQ ID NO: 127 and the p35 subunit of IL-12 comprises the amino acid sequence of SEQ ID NO: 128.

244. The kit, the use, or the method of claim 243, wherein the p35 subunit of IL-12 is fused to the second Fc region by a linker comprising the amino acid sequence of SEQ ID NO: 108.

245. The kit, the use, or the method of claim 244, wherein
(a) the first Fc region comprises mutations L234A, L235A, P329A, Y349C, K360E, and K409W, and
(b) the second Fc region comprises mutations L234A, L235A, P329A, Q347R, S354C, D399V, and F405T.

246. The kit, the use, or the method of claim 245, wherein
(a) the first Fc region comprises the amino acid sequence of SEQ ID NO:215, and
(b) the second Fc region comprises the amino acid sequence of SEQ ID NO:216.

247. The kit, the use, or the method of claim 246, wherein the first Fc region linked to the p40 subunit of IL12 comprises the amino acid sequence of SEQ ID NO:290 and the second Fc region linked to the p35 subunit of IL-12 comprises the amino acid sequence of SEQ ID NO:291.

248. The kit, the use, or the method of any one of claims 239-247, wherein the pharmaceutical formulation comprises: (a) citrate; (b) a sugar; (c) a sugar alcohol; and (d) a non-ionic surfactant, further wherein the pH of the formulation is between about 6.0 and about 7.0.

249. The kit, the use, or the method of claim 248, wherein the concentration of citrate in the pharmaceutical formulation is about 10 mM to about 30 mM.

250. The kit, the use, or the method of claim 249, wherein the concentration of citrate in the pharmaceutical formulation is about 20 mM.

251. The kit, the use, or the method of any one of claims 248-250, wherein the concentration of the sugar in the pharmaceutical formulation is about 3% to about 12% (w/v).

252. The kit, the use, or the method of claim 251, wherein the concentration of the sugar in the pharmaceutical formulation is about 6% (w/v).

253. The kit, the use, or the method of claim 251 or 252, wherein the sugar is a disaccharide.

254. The kit, the use, or the method of claim 253, wherein the disaccharide is sucrose.

255. The kit, the use, or the method of any one of claims 248-254, wherein the concentration of the sugar alcohol in the pharmaceutical formulation is between about 0.5% to about 6% (w/v).

256. The kit, the use, or the method of claim 255, wherein the concentration of the sugar alcohol in the pharmaceutical formulation is about 1% (w/v).

257. The kit, the use, or the method of any one of claims 248-256, wherein the sugar alcohol is derived from a monosaccharide.

258. The kit, the use, or the method of claim 257, wherein the sugar alcohol is mannitol.

259. The kit, the use, or the method of any one of claims 248-258, wherein the concentration of the non-ionic surfactant in the pharmaceutical formulation is between about 0.005% to about 0.02% (w/v).

260. The kit, the use, or the method of claim 259, wherein the concentration of polysorbate 80 in the pharmaceutical formulation is about 0.01% (w/v).

261. The kit, the use, or the method of claim 259 or 260, wherein the non-ionic surfactant is a polysorbate.

262. The kit, the use, or the method of claim 261, wherein the polysorbate is polysorbate 80.

263. The kit, the use, or the method of any one of claims 248-262, wherein the pH is between about 6.1 and about 6.9.

264. The kit, the use, or the method of claim 263, wherein the pH is between about 6.2 and about 6.8.

265. The kit, the use, or the method of claim 264, wherein the pH is between about 6.3 and about 6.7.

266. The kit, the use, or the method of claim 265, wherein the pH is between about 6.4 and about 6.6.

267. The kit, the use, or the method of claim 266, wherein the pH is about 6.5.

268. The kit, the use, or the method of any one of claims 248-267, wherein the pharmaceutical formulation further comprising water.

269. The kit, the use, or the method of claim 268, wherein the water is Water for Injection, USP.

270. The kit, the use, or the method of any one of claims 248-269, wherein the pharmaceutical formulation comprises a bulk concentration of heterodimeric Fc-fused protein of about 1 g/L to about 10 g/L.

271. The kit, the use, or the method of claim 270, wherein the pharmaceutical formulation comprises a bulk concentration of heterodimeric Fc-fused protein of about 2 g/L to about 8 g/L.

272. The kit, the use, or the method of claim 271, wherein the pharmaceutical formulation comprises a bulk concentration of heterodimeric Fc-fused protein of about 4 g/L to about 6 g/L.

273. The kit, the use, or the method of claim 272, wherein the pharmaceutical formulation comprises a bulk concentration of heterodimeric Fc-fused protein of about 5 g/L.

274. The kit, the use, or the method of any one of claims 248-273, wherein the pharmaceutical formulation comprises a concentration of the protein for administration of about 0.5 g/L to about 1.5 g/L.

275. The kit, the use, or the method of claim 274, wherein the pharmaceutical formulation comprises a concentration of the protein for administration of about 0.75 g/L to about 1.25 g/L.

276. The kit, the use, or the method of claim 275, wherein the pharmaceutical formulation comprises a concentration of the protein for administration of about 1 g/L.

277. The kit, the use, or the method of any one of 248-276, wherein the formulation is designed to be stored at a temperature between 2° C. and 8° C.

278. The kit, the use, or the method of any one of claims 248-277, wherein the pharmaceutical formulation is a clear, colorless solution and free of visible particulates.

279. The kit, the use, or the method of any one of claims 248-278, wherein the pharmaceutical formulation has a thermal stability profile as defined by:
(a) a Tₘ₁ of greater than about 60° C., greater than about 61° C., greater than about 62° C., greater than about 63° C., greater than about 64° C., greater than about 65° C., or greater than about 66° C.; and/or
(b) a Tₘ₂ of greater than about 70° C., greater than about 71° C., greater than about 72° C., greater than about 73° C., greater than about 74° C., greater than about 75° C., greater than about 76° C., or greater than about 77° C.,
as measured by differential scanning fluorimetry.

280. The kit, the use, or the method of claim 279, wherein the formulation has a thermal stability profile as defined by a Tₘ₁ of about 67.0° C. and a Tₘ₂ of about 77.3° C.

281. The kit, the use, or the method of claim 280, wherein the thermal stability profile of the pharmaceutical formulation, as defined by Tₘ₁ and/or Tₘ₂ is changed by less than about 2° C. or less than about 1° C. when the pharmaceutical formulation is incubated for 1 week at 50° C., as compared to the same pharmaceutical formulation that is incubated for 1 week at 5° C., as measured by differential scanning fluorimetry.

282. The kit, the use, or the method of any one of 248-281, wherein the formulation has a thermal stability profile as defined by a T_{agg} of greater than 60° C., greater than about 61° C., greater than about 62° C., greater than about 63° C., greater than about 64° C., greater than about 65° C., greater than about 66° C., or greater than about 67° C., as measured by differential scanning fluorimetry.

283. The kit, the use, or the method of claim 282, wherein the thermal stability profile of the pharmaceutical formulation, as defined by T_{agg} is changed by less than about 2° C. or less than about 1° C. when the pharmaceutical formulation is incubated for 1 week at 50° C., as compared to the same pharmaceutical formulation that is incubated for 1 week at 5° C., as measured by differential scanning fluorimetry.

284. The kit, the use, or the method of any one of claims 248-283, wherein the pH of the pharmaceutical formulation does not change by more than about 0.2 or about 0.1 in pH value after the pharmaceutical formulation is incubated for 1 week at 5° C.

285. The kit, the use, or the method of any one of claims 248-284, wherein the pH of the pharmaceutical formulation does not change by more than about 0.2 or about 0.1 in pH value after the pharmaceutical formulation is incubated for 1 week at 50° C.

286. The kit, the use, or the method of any one of claims 248-285, wherein the heterodimeric Fc-fused protein in the pharmaceutical formulation has a Z-average hydrodynamic diameter of less than about 15 nm, less than about 14 nm, less than about 13 nm, or less than about 12 nm, as measured by dynamic light scattering at 25° C.

287. The kit, the use, or the method of claim 286, wherein the heterodimeric Fc-fused protein in the pharmaceutical formulation has a Z-average hydrodynamic diameter of about 11.6 nm.

288. The kit, the use, or the method of any one of claims 248-287, wherein the heterodimeric Fc-fused protein in the pharmaceutical formulation has a Z-average hydrodynamic diameter of less than about 20 nm, less than about 19 nm, less than about 18 nm, less than about 17 nm, less than about 16 nm, or less than about 15 nm, as measured by dynamic light scattering at 25° C., after the pharmaceutical formulation is incubated for 2 weeks at 50° C.

289. The kit, the use, or the method of claim 288, wherein the heterodimeric Fc-fused protein in the pharmaceutical formulation has a Z-average hydrodynamic diameter of about 14.4 nm.

290. The kit, the use, or the method of any one of claims 248-289, wherein the heterodimeric Fc-fused protein in the pharmaceutical formulation has a Z-average hydrodynamic diameter of less than about 20 nm, less than about 19 nm, less than about 18 nm, less than about 17 nm, or less than about 16 nm, as measured by dynamic light scattering at 25° C., after the pharmaceutical formulation is subjected to five freeze thaw cycles.

291. The kit, the use, or the method of claim 290, wherein the heterodimeric Fc-fused protein in the pharmaceutical formulation has a Z-average hydrodynamic diameter of about 15.3 nm.

292. The kit, the use, or the method of any one of claims 248-291, wherein the polydispersity index of the heterodimeric Fc-fused protein in the pharmaceutical formulation is less than about 0.30, less than about 0.29, less than about 0.28, or less than about 0.27, as measured by dynamic light scattering at 25° C.

293. The kit, the use, or the method of any one of claims 248-292, wherein the polydispersity index of the heterodimeric Fc-fused protein in the pharmaceutical formulation is about 0.26.

294. The kit, the use, or the method of any one of claims 248-293, wherein the polydispersity index of the heterodimeric Fc-fused protein in the pharmaceutical formulation is less than about 0.30, less than about 0.29, less than about 0.28, less than about 0.27, or less than about 0.26, as measured by dynamic light scattering at 25° C., after the pharmaceutical formulation is incubated for 2 weeks at 50° C.

295. The kit, the use, or the method of any one of claims 248-294, wherein the polydispersity index of the heterodimeric Fc-fused protein in the pharmaceutical formulation is about 0.25.

296. The kit, the use, or the method of any one of claims 248-295, wherein the polydispersity index of the heterodimeric Fc-fused protein in the pharmaceutical formulation is less than about 0.40, less than about 0.35, or less than about 0.34, as measured by dynamic light scattering at 25° C., after the pharmaceutical formulation is subjected to five freeze thaw cycles.

297. The kit, the use, or the method of any one of claims 248-296, wherein the polydispersity index of the heterodimeric Fc-fused protein in the pharmaceutical formulation is about 0.33.

298. The kit, the use, or the method of any one of claims 248-297, wherein the purity profile of the pharmaceutical formulation, as measured by the area of the main peak as a percentage of total detected area in a SEC-HPLC analysis, is greater than about 90%, greater than about 91%, greater than about 92%, greater than about 93%, greater than about 94%, greater than about 95%, greater than about 96%, greater than about 97%, greater than about 98%, or greater than about 99%.

299. The kit, the use, or the method of claim 298, wherein the purity profile of the pharmaceutical formulation, as measured by the area of the main peak as a percentage of total detected area in a SEC-HPLC analysis, is about 99.0%.

300. The kit, the use, or the method of any one of claims 248-299, wherein the purity profile of the pharmaceutical formulation, as measured by the area of the main peak as a percentage of total detected area in a SEC-HPLC analysis, is greater than about 75%, greater than about 80%, greater than about 81%, greater than about 82%, greater than about 83%, greater than about 84%, or greater than about 85%, after the pharmaceutical formulation is incubated for 2 weeks at 50° C.

301. The kit, the use, or the method of claim 300, wherein the purity profile of the pharmaceutical formulation, as measured by the area of the main peak as a percentage of total detected area in a SEC-HPLC analysis, is about 85.2%.

302. The kit, the use, or the method of any one of claims 248-301, wherein the purity profile of the pharmaceutical formulation, as measured by the area of the main peak as a percentage of total detected area in a SEC-HPLC analysis, is greater than about 90%, greater than about 91%, greater than about 92%, greater than about 93%, greater than about 94%, greater than about 95%, greater than about 96%, greater than about 97%, or greater than about 98%, after the pharmaceutical formulation is subjected to five freeze thaw cycles.

303. The kit, the use, or the method of claim 302, wherein the purity profile of the pharmaceutical formulation, as measured by the area of the main peak as a percentage of total detected area in a SEC-HPLC analysis, is about 98.9%.
